SEQUENCE LISTING (180 sequences)

SEQ ID NO: 1 - protein, 5 aa, synthetic construct
  REGION 1..5: note = Synthetic construct
RYWMN

SEQ ID NO: 2 - protein, 17 aa, synthetic construct
  REGION 1..17: note = Synthetic construct
EITPDSSTINYTPSLKD

SEQ ID NO: 3 - protein, 10 aa, synthetic construct
  REGION 1..10: note = Synthetic construct
PYDYGAWFAS

SEQ ID NO: 4 - protein, 14 aa, synthetic construct
  REGION 1..14: note = Synthetic construct
RSSTGAVTTSNYAN

SEQ ID NO: 5 - protein, 7 aa, synthetic construct
  REGION 1..7: note = Synthetic construct
GTNKRAP

SEQ ID NO: 6 - protein, 9 aa, synthetic construct
  REGION 1..9: note = Synthetic construct
ALWYSNHWV

SEQ ID NO: 7 - protein, 119 aa, synthetic construct
  REGION 1..119: note = Synthetic construct
EVKLLESGGGLVQPGGSLKLSCAASGFDFSRYWMNWVRQAPGKGLEWIGEITPDSSTINYTPSLKDKFIISRDNAKNTLYLQMIKVRSEDTALYYCVRPYDYGAWFASWGQGTLVTVSA

SEQ ID NO: 8 - protein, 109 aa, synthetic construct
  REGION 1..109: note = Synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNHWVFGGGTKLTVL

SEQ ID NO: 9 - protein, 447 aa, synthetic construct
  REGION 1..447: note = Synthetic construct
EVKLLESGGGLVQPGGSLKLSCAASGFDFSRYWMNWVRQAPGKGLEWIGEITPDSSTINYTPSLKDKFIISRDNAKNTLYLQMIKVRSEDTALYYCVRPYDYGAWFASWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 10 - protein, 215 aa, synthetic construct
  REGION 1..215: note = Synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 17 aa, synthetic construct
  REGION 1..17: note = Synthetic construct
EITPDSSTINYTPSLKG

SEQ ID NO: 12 - protein, 119 aa, synthetic construct
  REGION 1..119: note = Synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSS

SEQ ID NO: 13 - protein, 109 aa, synthetic construct
  REGION 1..109: note = Synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVL

SEQ ID NO: 14 - protein, 447 aa, synthetic construct
  REGION 1..447: note = Synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 15 - protein, 215 aa, synthetic construct
  REGION 1..215: note = Synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 16 - protein, 17 aa, synthetic construct
  REGION 1..17: note = Synthetic construct
EITPDSSTINYAPSLKG

SEQ ID NO: 17 - protein, 119 aa, synthetic construct
  REGION 1..119: note = Synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSS

SEQ ID NO: 18 - protein, 447 aa, synthetic construct
  REGION 1..447: note = Synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 19 - protein, 119 aa, synthetic construct
  REGION 1..119: note = Synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSS

SEQ ID NO: 20 - protein, 447 aa, synthetic construct
  REGION 1..447: note = Synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 21 - protein, 17 aa, synthetic construct
  REGION 1..17: note = Synthetic construct
EITPDSSTINYADSVKG

SEQ ID NO: 22 - protein, 119 aa, synthetic construct
  REGION 1..119: note = Synthetic construct
EVQLVESGGGLVQPGGSLRLSCAASGFDFSRYWMNWVRQAPGKGLEWVSEITPDSSTINYADSVKGRFTISRDNAKNSLYLQMNSLRAEDTAVYYCARPYDYGAWFASWGQGTLVTVSS

SEQ ID NO: 23 - protein, 447 aa, synthetic construct
  REGION 1..447: note = Synthetic construct
EVQLVESGGGLVQPGGSLRLSCAASGFDFSRYWMNWVRQAPGKGLEWVSEITPDSSTINYADSVKGRFTISRDNAKNSLYLQMNSLRAEDTAVYYCARPYDYGAWFASWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 24 - protein, 109 aa, synthetic construct
  REGION 1..109: note = Synthetic construct
QAVVTQEPSLTVSPGGTVTLTCRSSTGAVTTSNYANWFQQKPGQAFTGLIGGTNKRAPGTPARFSGSLLGGKAALTLSGAQPEDEAEYYCALWYSNHWVFGGGTKLTVL

SEQ ID NO: 25 - protein, 215 aa, synthetic construct
  REGION 1..215: note = Synthetic construct
QAVVTQEPSLTVSPGGTVTLTCRSSTGAVTTSNYANWFQQKPGQAFTGLIGGTNKRAPGTPARFSGSLLGGKAALTLSGAQPEDEAEYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - protein, 14 aa, synthetic construct
  REGION 1..14: note = Synthetic construct
GSSTGAVTTSNYAN

SEQ ID NO: 27 - protein, 109 aa, synthetic construct
  REGION 1..109: note = Synthetic construct
QAVVTQEPSLTVSPGGTVTLTCGSSTGAVTTSNYANWFQQKPGQAPRTLIGGTNKRAPGTPARFSGSLLGGKAALTLSGAQPEDEAEYYCALWYSNHWVFGGGTKLTVL

SEQ ID NO: 28 - protein, 215 aa, synthetic construct
  REGION 1..215: note = Synthetic construct
QAVVTQEPSLTVSPGGTVTLTCGSSTGAVTTSNYANWFQQKPGQAPRTLIGGTNKRAPGTPARFSGSLLGGKAALTLSGAQPEDEAEYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 29 - protein, 230 aa, synthetic construct
  REGION 1..230: note = Synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 30 - protein, 230 aa, synthetic construct
  REGION 1..230: note = Synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALLAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 31 - protein, 230 aa, synthetic construct
  REGION 1..230: note = Synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALIAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 32 - protein, 230 aa, synthetic construct
  REGION 1..230: note = Synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 33 - protein, 230 aa, synthetic construct
  REGION 1..230: note = Synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 34 - protein, 5 aa, synthetic construct
  REGION 1..5: note = Synthetic construct
TYAMN

SEQ ID NO: 35 - protein, 5 aa, synthetic construct
  REGION 1..5: note = Synthetic construct
SYAMN

SEQ ID NO: 36 - protein, 5 aa, synthetic construct
  REGION 1..5: note = Synthetic construct
NYAMN

SEQ ID NO: 37 - protein, 19 aa, synthetic construct
  REGION 1..19: note = Synthetic construct
RIRSKYNNYATYYADSVKG

SEQ ID NO: 38 - protein, 19 aa, synthetic construct
  REGION 1..19: note = Synthetic construct
RIRSKYNEYATYYADSVKG

SEQ ID NO: 39 - protein, 19 aa, synthetic construct
  REGION 1..19: note = Synthetic construct
RIRSKHNGYATYYADSVKG

SEQ ID NO: 40 - protein, 19 aa, synthetic construct
  REGION 1..19: note = Synthetic construct
RIRTKYNEYATYYADSVKG

SEQ ID NO: 41 - protein, 14 aa, synthetic construct
  REGION 1..14: note = Synthetic construct
HGNFGNSYVSWFAY

SEQ ID NO: 42 - protein, 14 aa, synthetic construct
  REGION 1..14: note = Synthetic construct
ASNFPSSFVSYFGY

SEQ ID NO: 43 - protein, 14 aa, synthetic construct
  REGION 1..14: note = Synthetic construct
ASNFPASYVSYFAY

SEQ ID NO: 44 - protein, 14 aa, synthetic construct
  REGION 1..14: note = Synthetic construct
ASNFPSSYVSYFGY

SEQ ID NO: 45 - protein, 14 aa, synthetic construct
  REGION 1..14: note = Synthetic construct
ASNFPSSYVSYFAY

SEQ ID NO: 46 - protein, 14 aa, synthetic construct
  REGION 1..14: note = Synthetic construct
ASNFPQSYVSYFGY

SEQ ID NO: 47 - protein, 125 aa, synthetic construct
  REGION 1..125: note = Synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 48 - protein, 125 aa, synthetic construct
  REGION 1..125: note = Synthetic construct
EVQLLESGGGLVQPGGSLRLSCAASGFTFESYAMNWVRQAPGKGLEWVSRIRSKYNEYATYYADSVKGRFTISRDDSKNTLYLQMNSLRAEDTAVYYCVRASNFPSSFVSYFGYWGQGTLVTVSS

SEQ ID NO: 49 - protein, 125 aa, synthetic construct
  REGION 1..125: note = Synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSS

SEQ ID NO: 50 - protein, 125 aa, synthetic construct
  REGION 1..125: note = Synthetic construct
EVQLLESGGGLVQPGGSLRLSCAASGFDFDNYAMNWVRQAPGKGLEWVSRIRSKHNGYATYYADSVKGRFTISRDDSKNTLYLQMNSLRAEDTAVYYCVRASNFPSSYVSYFGYWGQGTLVTVSS

SEQ ID NO: 51 - protein, 125 aa, synthetic construct
  REGION 1..125: note = Synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPSSYVSYFAYWGQGTLVTVSS

SEQ ID NO: 52 - protein, 125 aa, synthetic construct
  REGION 1..125: note = Synthetic construct
EVQLLESGGGLVQPGGSLRLSCAASGFQFDNYAMNWVRQAPGKGLEWVSRIRTKYNEYATYYADSVKGRFTISRDDSKNTLYLQMNSLRAEDTAVYYCVRASNFPQSYVSYFGYWGQGTLVTVSS

SEQ ID NO: 53 - protein, 14 aa, synthetic construct
  REGION 1..14: note = Synthetic construct
GSSTGAVTTSNYAN

SEQ ID NO: 54 - protein, 7 aa, synthetic construct
  REGION 1..7: note = Synthetic construct
GTNKRAP

SEQ ID NO: 55 - protein, 9 aa, synthetic construct
  REGION 1..9: note = Synthetic construct
ALWYSNLWV

SEQ ID NO: 56 - protein, 109 aa, synthetic construct
  REGION 1..109: note = Synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 57 - protein, 5 aa, synthetic construct
  REGION 1..5: note = Synthetic construct
DYFLH

SEQ ID NO: 58 - protein, 17 aa, synthetic construct
  REGION 1..17: note = Synthetic construct
WINPDNGNTVYAQKFQG

SEQ ID NO: 59 - protein, 12 aa, synthetic construct
  REGION 1..12: note = Synthetic construct
RDYTYEKAALDY

SEQ ID NO: 60 - protein, 121 aa, synthetic construct
  REGION 1..121: note = Synthetic construct
QVQLVQSGAEVKKPGASVKVSCKASGFNIKDYFLHWVRQAPGQGLEWMGWINPDNGNTVYAQKFQGRVTMTADTSTSTVYMELSSLRSEDTAVYYCTRRDYTYEKAALDYWGQGTLVTVSS

SEQ ID NO: 61 - protein, 11 aa, synthetic construct
  REGION 1..11: note = Synthetic construct
RASGNIYNYLA

SEQ ID NO: 62 - protein, 7 aa, synthetic construct
  REGION 1..7: note = Synthetic construct
DAKTLAD

SEQ ID NO: 63 - protein, 9 aa, synthetic construct
  REGION 1..9: note = Synthetic construct
QHFWSLPFT

SEQ ID NO: 64 - protein, 107 aa, synthetic construct
  REGION 1..107: note = Synthetic construct
DIQMTQSPSSLSASVGDRVTITCRASGNIYNYLAWYQQKPGKVPKLLIYDAKTLADGVPSRFSGSGSGTDFTLTISSLQPEDVATYYCQHFWSLPFTFGQGTKLEIK

SEQ ID NO: 65 - protein, 674 aa, synthetic construct
  REGION 1..674: note = Synthetic construct
QVQLVQSGAEVKKPGASVKVSCKASGFNIKDYFLHWVRQAPGQGLEWMGWINPDNGNTVYAQKFQGRVTMTADTSTSTVYMELSSLRSEDTAVYYCTRRDYTYEKAALDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 66 - protein, 449 aa, synthetic construct
  REGION 1..449: note = Synthetic construct
QVQLVQSGAEVKKPGASVKVSCKASGFNIKDYFLHWVRQAPGQGLEWMGWINPDNGNTVYAQKFQGRVTMTADTSTSTVYMELSSLRSEDTAVYYCTRRDYTYEKAALDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 67 - protein, 214 aa, synthetic construct
  REGION 1..214: note = Synthetic construct
DIQMTQSPSSLSASVGDRVTITCRASGNIYNYLAWYQQKPGKVPKLLIYDAKTLADGVPSRFSGSGSGTDFTLTISSLQPEDVATYYCQHFWSLPFTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 232 aa, synthetic construct
  REGION 1..232: note = Synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 232 aa, synthetic construct
  REGION 1..232: note = Synthetic construct
EVQLLESGGGLVQPGGSLRLSCAASGFTFESYAMNWVRQAPGKGLEWVSRIRSKYNEYATYYADSVKGRFTISRDDSKNTLYLQMNSLRAEDTAVYYCVRASNFPSSFVSYFGYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 232 aa, synthetic construct
  REGION 1..232: note = Synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 232 aa, synthetic construct
  REGION 1..232: note = Synthetic construct
EVQLLESGGGLVQPGGSLRLSCAASGFDFDNYAMNWVRQAPGKGLEWVSRIRSKHNGYATYYADSVKGRFTISRDDSKNTLYLQMNSLRAEDTAVYYCVRASNFPSSYVSYFGYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 232 aa, synthetic construct
  REGION 1..232: note = Synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPSSYVSYFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 232 aa, synthetic construct
  REGION 1..232: note = Synthetic construct
EVQLLESGGGLVQPGGSLRLSCAASGFQFDNYAMNWVRQAPGKGLEWVSRIRTKYNEYATYYADSVKGRFTISRDDSKNTLYLQMNSLRAEDTAVYYCVRASNFPQSYVSYFGYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 360 aa, synthetic construct [sequence omitted]

SEQ ID NO: 75 - protein, 325 aa, synthetic construct
  REGION 1..325: note = Synthetic construct
FKIPIEELEDRVFVNCNTSITWVEGTVGTLLSDITRLDLGKRILDPRGIYRCNGTDIYKDKESTVQVHYRMCRSEQLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKSGGLNDIFEAQKIEWHE

SEQ ID NO: 76 - protein, 351 aa, synthetic construct
  REGION 1..351: note = Synthetic construct
QDGNEEMGSITQTPYQVSISGTTVILTCSQHLGSEAQWQHNGKNKEDSGDRLFLPEFSEMEQSGYYVCYPRGSNPEDASHHLYLKARVSENCVDEQLYFQGGSPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 77 - protein, 334 aa, synthetic construct
  REGION 1..334: note = Synthetic construct
FKIPVEELEDRVFVKCNTSVTWVEGTVGTLLTNNTRLDLGKRILDPRGIYRCNGTDIYKDKESAVQVHYRMSQNCVDEQLYFQGGSPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKSGGLNDIFEAQKIEWHE

SEQ ID NO: 78 - protein, 186 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 79 - protein, 177 aa, Cynomolgus monkey
QDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 80 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 81 - protein, 10 aa, synthetic construct
  REGION 1..10: note = Synthetic construct
GGGGSGGGGS

SEQ ID NO: 82 - protein, 11 aa, synthetic construct
  REGION 1..11: note = Synthetic construct
DGGGGSGGGGS

SEQ ID NO: 83 - protein, 107 aa, Homo sapiens [sequence omitted]

SEQ ID NO: 84 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 85 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 86 - protein, 215 aa, synthetic construct
  REGION 1..215: note = Synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNHWVFGGGTKLTVLGQPKAAPSVTLFPPSSKKLQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 87 - protein, 672 aa, synthetic construct
  REGION 1..672: note = Synthetic construct
EVKLLESGGGLVQPGGSLKLSCAASGFDFSRYWMNWVRQAPGKGLEWIGEITPDSSTINYTPSLKDKFIISRDNAKNTLYLQMIKVRSEDTALYYCVRPYDYGAWFASWGQGTLVTVSAASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 88 - protein, 447 aa, synthetic construct
  REGION 1..447: note = Synthetic construct
EVKLLESGGGLVQPGGSLKLSCAASGFDFSRYWMNWVRQAPGKGLEWIGEITPDSSTINYTPSLKDKFIISRDNAKNTLYLQMIKVRSEDTALYYCVRPYDYGAWFASWGQGTLVTVSAASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 89 - protein, 215 aa, synthetic construct
  REGION 1..215: note = Synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGQPKAAPSVTLFPPSSKKLQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 90 - protein, 447 aa, synthetic construct
  REGION 1..447: note = Synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 91 - protein, 439 aa, synthetic construct
  REGION 1..439: note = Synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 92 - protein, 672 aa, synthetic construct
  REGION 1..672: note = Synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGGQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 93 - protein, 216 aa, synthetic construct
  REGION 1..216: note = Synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNHWVFGGGTKLTVLRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 5 aa, synthetic construct
  REGION 1..5: note = Synthetic construct
SYYIH

SEQ ID NO: 95 - protein, 17 aa, synthetic construct
  REGION 1..17: note = Synthetic construct
SIYPGNVQTNYNEKFKD

SEQ ID NO: 96 - protein, 11 aa, synthetic construct
  REGION 1..11: note = Synthetic construct
SHYGLDWNFDV

SEQ ID NO: 97 - protein, 11 aa, synthetic construct
  REGION 1..11: note = Synthetic construct
HASQNIYVFLN

SEQ ID NO: 98 - protein, 7 aa, synthetic construct
  REGION 1..7: note = Synthetic construct
KASNLHT

SEQ ID NO: 99 - protein, 9 aa, synthetic construct
  REGION 1..9: note = Synthetic construct
QQGQTYPYT

SEQ ID NO: 100 - protein, 120 aa, synthetic construct
  REGION 1..120: note = Synthetic construct
QVQLVQSGAEVKKPGASVKVSCKASGYTFTSYYIHWVRQAPGQGLEWIGSIYPGNVQTNYNEKFKDRATLTVDTSISTAYMELSRLRSDDTAVYFCTRSHYGLDWNFDVWGQGTTVTVSS

SEQ ID NO: 101 - protein, 107 aa, synthetic construct
  REGION 1..107: note = Synthetic construct
DIQMTQSPSSLSASVGDRVTITCHASQNIYVFLNWYQQKPGKAPKLLIYKASNLHTGVPSRFSGSGSGTDFTLTISSLQPEDFATYYCQQGQTYPYTFGGGTKVEIK

SEQ ID NO: 102 - protein, 11 aa, synthetic construct
  REGION 1..11: note = Synthetic construct
SHYGLDFNFDV

SEQ ID NO: 103 - protein, 11 aa, synthetic construct
  REGION 1..11: note = Synthetic construct
HASQNIYVYLN

SEQ ID NO: 104 - protein, 120 aa, synthetic construct
  REGION 1..120: note = Synthetic construct
QVQLVQSGAEVKKPGASVKVSCKASGYTFTSYYIHWVRQAPGQGLEWIGSIYPGNVQTNYNEKFKDRATLTVDTSISTAYMELSRLRSDDTAVYFCTRSHYGLDFNFDVWGQGTTVTVSS

SEQ ID NO: 105 - protein, 107 aa, synthetic construct
  REGION 1..107: note = Synthetic construct
DIQMTQSPSSLSASVGDRVTITCHASQNIYVYLNWYQQKPGKAPKLLIYKASNLHTGVPSRFSGSGSGTDFTLTISSLQPEDFATYYCQQGQTYPYTFGGGTKVEIK

SEQ ID NO: 106 - protein, 227 aa, synthetic construct
  REGION 1..227: note = Synthetic construct
QVQLVQSGAEVKKPGASVKVSCKASGYTFTSYYIHWVRQAPGQGLEWIGSIYPGNVQTNYNEKFKDRATLTVDTSISTAYMELSRLRSDDTAVYFCTRSHYGLDWNFDVWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 437 aa, synthetic construct
  REGION 1..437: note = Synthetic construct
DIQMTQSPSSLSASVGDRVTITCHASQNIYVFLNWYQQKPGKAPKLLIYKASNLHTGVPSRFSGSGSGTDFTLTISSLQPEDFATYYCQQGQTYPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 108 - protein, 227 aa, synthetic construct
  REGION 1..227: note = Synthetic construct
QVQLVQSGAEVKKPGASVKVSCKASGYTFTSYYIHWVRQAPGQGLEWIGSIYPGNVQTNYNEKFKDRATLTVDTSISTAYMELSRLRSDDTAVYFCTRSHYGLDFNFDVWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 437 aa, synthetic construct
  REGION 1..437: note = Synthetic construct
DIQMTQSPSSLSASVGDRVTITCHASQNIYVYLNWYQQKPGKAPKLLIYKASNLHTGVPSRFSGSGSGTDFTLTISSLQPEDFATYYCQQGQTYPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 110 - protein, 226 aa, synthetic construct
  REGION 1..226: note = Synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 214 aa, synthetic construct
  REGION 1..214: note = Synthetic construct
DIQMTQSPSSLSASVGDRVTITCHASQNIYVYLNWYQQKPGKAPKLLIYKASNLHTGVPSRFSGSGSGTDFTLTISSLQPEDFATYYCQQGQTYPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 439 aa, synthetic construct
  REGION 1..439: note = Synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 113 - protein, 448 aa, synthetic construct
  REGION 1..448: note = Synthetic construct
QVQLVQSGAEVKKPGASVKVSCKASGYTFTSYYIHWVRQAPGQGLEWIGSIYPGNVQTNYNEKFKDRATLTVDTSISTAYMELSRLRSDDTAVYFCTRSHYGLDFNFDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 114 - protein, 670 aa, synthetic construct
  REGION 1..670: note = Synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGGDIQMTQSPSSLSASVGDRVTITCHASQNIYVYLNWYQQKPGKAPKLLIYKASNLHTGVPSRFSGSGSGTDFTLTISSLQPEDFATYYCQQGQTYPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 115 - protein, 670 aa, synthetic construct
  REGION 1..670: note = Synthetic construct
DIQMTQSPSSLSASVGDRVTITCHASQNIYVYLNWYQQKPGKAPKLLIYKASNLHTGVPSRFSGSGSGTDFTLTISSLQPEDFATYYCQQGQTYPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGGEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 116 - protein, 374 aa, synthetic construct
  REGION 1..374: note = Synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 117 - protein, 184 aa, Homo sapiens
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 118 - protein, 170 aa, Homo sapiens
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 119 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 120 - protein, 203 aa, Homo sapiens
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 121 - protein, 178 aa, Homo sapiens
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 122 - protein, 164 aa, Homo sapiens
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 123 - protein, 169 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 124 - protein, 197 aa, Homo sapiens
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 125 - protein, 378 aa, synthetic construct
  REGION 1..378: note = Synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 126 - protein, 366 aa, synthetic construct
  REGION 1..366: note = Synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 127 - protein, 360 aa, synthetic construct
  REGION 1..360: note = Synthetic construct
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 128 - protein, 416 aa, synthetic construct
  REGION 1..416: note = Synthetic construct
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 129 - protein, 291 aa, synthetic construct
  REGION 1..291: note = Synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 130 - protein, 708 aa, synthetic construct
  REGION 1..708: note = Synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 131 - protein, 447 aa, synthetic construct
  REGION 1..447: note = Synthetic construct
EVKLLESGGGLVQPGGSLKLSCAASGFDFSRYWMNWVRQAPGKGLEWIGEITPDSSTINYTPSLKDKFIISRDNAKNTLYLQMIKVRSEDTALYYCVRPYDYGAWFASWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 132 - protein, 447 aa, synthetic construct
  REGION 1..447: note = Synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 133 - protein, 5 aa, synthetic construct
  REGION 1..5: note = Synthetic construct
GYYWS

SEQ ID NO: 134 - protein, 16 aa, synthetic construct
  REGION 1..16: note = Synthetic construct
EINHGGYVTYNPSLES

SEQ ID NO: 135 - protein, 13 aa, synthetic construct
  REGION 1..13: note = Synthetic construct
DYGPGNYDWYFDL

SEQ ID NO: 136 - protein, 11 aa, synthetic construct
  REGION 1..11: note = Synthetic construct
RASQSVSSYLA

SEQ ID NO: 137 - protein, 7 aa, synthetic construct
  REGION 1..7: note = Synthetic construct
DASNRAT

SEQ ID NO: 138 - protein, 11 aa, synthetic construct
  REGION 1..11: note = Synthetic construct
QQRSNWPPALT

SEQ ID NO: 139 - protein, 121 aa, synthetic construct [sequence omitted]

SEQ ID NO: 140 - protein, 109 aa, synthetic construct [sequence omitted]

SEQ ID NO: 141 - protein, 226 aa, synthetic construct
  REGION 1..226: note = Synthetic construct
EVKLLESGGGLVQPGGSLKLSCAASGFDFSRYWMNWVRQAPGKGLEWIGEITPDSSTINYTPSLKDKFIISRDNAKNTLYLQMIKVRSEDTALYYCVRPYDYGAWFASWGQGTLVTVSAASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 216 aa, synthetic construct
  REGION 1..216: note = Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 672 aa, synthetic construct
  REGION 1..672: note = Synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNHWVFGGGTKLTVLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 144 - protein, 449 aa, synthetic construct
  REGION 1..449: note = Synthetic construct
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 145 - protein, 672 aa, synthetic construct
  REGION 1..672: note = Synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 146 - protein, 446 aa, synthetic construct
  REGION 1..446: note = Synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 147 - protein, 210 aa, synthetic construct
  REGION 1..210: note = Synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFN

SEQ ID NO: 148 - protein, 447 aa, synthetic construct
  REGION 1..447: note = Synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 149 - protein, 219 aa, synthetic construct
  REGION 1..219: note = Synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 448 aa, synthetic construct
  REGION 1..448: note = Synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 151 - protein, 215 aa, synthetic construct
  REGION 1..215: note = Synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 152 - protein, 450 aa, synthetic construct
  REGION 1..450: note = Synthetic construct
QVQLVQSGAEVKKPGSSVKVSCKASGGTFSSLAISWVRQAPGQGLEWMGGIIPIFGTANYAQKFQGRVTITADESTSTAYMELSSLRSEDTAVYYCARGGSVSGTLVDFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 153 - protein, 214 aa, synthetic construct
  REGION 1..214: note = Synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNIYPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 445 aa, synthetic construct
  REGION 1..445: note = Synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 155 - protein, 215 aa, synthetic construct
  REGION 1..215: note = Synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 443 aa, synthetic construct
  REGION 1..443: note = Synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 157 - protein, 216 aa, synthetic construct
  REGION 1..216: note = Synthetic construct
EIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 452 aa, synthetic construct
  REGION 1..452: note = Synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYEDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 159 - protein, 220 aa, synthetic construct
  REGION 1..220: note = Synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 448 aa, synthetic construct
  REGION 1..448: note = Synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 161 - protein, 218 aa, synthetic construct
  REGION 1..218: note = Synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 449 aa, synthetic construct
  REGION 1..449: note = Synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 214 aa, synthetic construct
  REGION 1..214: note = Synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 451 aa, synthetic construct
  REGION 1..451: note = Synthetic construct
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 218 aa, synthetic construct
  REGION 1..218: note = Synthetic construct
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 133 aa, Homo sapiens [sequence omitted]

SEQ ID NO: 167 - protein, 133 aa, synthetic construct
  REGION 1..133: note = Synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 168 - protein, 5 aa, synthetic construct
  REGION 1..5: note = Synthetic construct
GYYWS

SEQ ID NO: 169 - protein, 16 aa, synthetic construct
  REGION 1..16: note = Synthetic construct
EINHGGYVTYNPSLES

SEQ ID NO: 170 - protein, 13 aa, synthetic construct
  REGION 1..13: note = Synthetic construct
DYGPGNYDWYFDL

SEQ ID NO: 171 - protein, 11 aa, synthetic construct
  REGION 1..11: note = Synthetic construct
RASQSVSSYLA

SEQ ID NO: 172 - protein, 7 aa, synthetic construct
  REGION 1..7: note = Synthetic construct
DASNRAT

SEQ ID NO: 173 - protein, 11 aa, synthetic construct
  REGION 1..11: note = Synthetic construct
QQRSNWPPALT

SEQ ID NO: 174 - protein, 140 aa, synthetic construct
  REGION 1..140: note = Synthetic construct
MNFGLSLVFLALILKGVQCEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSS

SEQ ID NO: 175 - protein, 112 aa, synthetic construct
  REGION 1..112: note = Synthetic construct
DVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIK

SEQ ID NO: 176 - protein, 14 aa, synthetic construct
  REGION 1..14: note = clone 22 HCDR3
HTTFPSSYVSYYGY

SEQ ID NO: 177 - protein, 125 aa, synthetic construct
  REGION 1..125: note = clone 22 VH
EVQLLESGGGLVQPGGSLRLSCAASGFQFSSYAMNWVRQAPGKGLEWVSRIRSKYNNYATYYADSVKGRFTISRDDSKNTLYLQMNSLRAEDTAVYYCVRHTTFPSSYVSYYGYWGQGTLVTVSS

SEQ ID NO: 178 - protein, 447 aa, synthetic construct
  REGION 1..447: note = Synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 179 - protein, 672 aa, synthetic construct
  REGION 1..672: note = Synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGGQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 180 - protein, 232 aa, synthetic construct
  REGION 1..232: note = Synthetic construct
EVQLLESGGGLVQPGGSLRLSCAASGFQFSSYAMNWVRQAPGKGLEWVSRIRSKYNNYATYYADSVKGRFTISRDDSKNTLYLQMNSLRAEDTAVYYCVRHTTFPSSYVSYYGYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0043535 A1
(43) Pub. Date: Feb. 8, 2024

(54) IMMUNE ACTIVATING Fc DOMAIN BINDING MOLECULES

(71) Applicant: Hoffmann-La Roche Inc., Little Falls, NJ (US)

(72) Inventors: Maria Amann, Zürich (CH); Alejandro Carpy Gutierrez Cirlos, München (DE); Christina Claus, Ennetbaden (CH); Laura Codarri Deak, Au (CH); Diana Darowski, Wuestenrot (DE); Tanja Fauti, Zuerich (CH); Claudia Ferrara Koller, Zug (CH); Anne Freimoser-Grundschober, Zürich (CH); Sylvia Herter, Baden-Rütihof (CH); Thomas Hofer, Zürich (CH); Christian Klein, Bonstetten (CH); Laura Lauener, Baden (CH); Stephane Leclair, Gauting (DE); Ekkehard Moessner, Kreuzlingen (CH); Christiane Neumann, Oberweningen (CH); Pablo Umaña, Wollerau (CH); Ali Bransi, Dietikon (CH); Marlena Surówka, Schlieren (CH)

(73) Assignee: Hoffmann-La Roche Inc., Little Falls, NJ (US)

(21) Appl. No.: 18/067,330

(22) Filed: Dec. 16, 2022

Related U.S. Application Data
(63) Continuation of application No. PCT/EP2021/066337, filed on Jun. 17, 2021.

(30) Foreign Application Priority Data
Jun. 19, 2020 (EP) 20181087.6

(51) Int. Cl. C07K 16/28; C07K 14/55; C07K 14/54; C07K 14/56; C07K 14/57; A61P 35/00

(52) U.S. Cl. CPC C07K 16/2809; C07K 16/2818; C07K 16/2878; C07K 14/55; C07K 14/5418; C07K 14/5443; C07K 14/56; C07K 14/57; C07K 14/54; C07K 16/283; A61P 35/00; C07K 2319/30

(57) ABSTRACT

The present invention generally relates to novel immune activating Fc domain binding molecules for activation of immune cells and re-direction to specific target cells. In addition, the present invention relates to polynucleotides encoding such molecules, and vectors and host cells comprising such polynucleotides. The invention further relates to methods for producing the bispecific antigen binding molecules of the invention, and to methods of using these bispecific antigen binding molecules in the treatment of disease.

### CROSS-REFERENCE TO RELATED APPLICATIONS

This application is a Continuation of International Patent Application No. PCT/EP2021/066337, filed Jun. 17, 2021, which claims benefit of priority to European Application No. 20181087.6, filed Jun. 19, 2020, each of which is incorporated herein by reference in its entirety.

### SEQUENCE LISTING

This application contains a Sequence Listing which has been submitted electronically in XML format and is hereby incorporated by reference in its entirety. Said XML copy, created on Dec. 12, 2022, is named P36106_US_sequence_listing.xml and is 254,102 bytes in size.

### FIELD OF THE INVENTION

The present invention generally relates to novel immune activating Fc domain binding molecules for activation of immune cells and re-direction to specific target cells. In addition, the present invention relates to polynucleotides encoding such molecules, and vectors and host cells comprising such polynucleotides. The invention further relates to methods for producing the bispecific antigen binding molecules of the invention, and to methods of using these bispecific antigen binding molecules in the treatment of disease.

### BACKGROUND

The selective destruction of an individual cell or a specific cell type is often desirable in a variety of clinical settings. For example, it is a primary goal of cancer therapy to specifically destroy tumor cells, while leaving healthy cells and tissues intact and undamaged, or to destroy certain cell subsets identified by a specific surface antigen.

An attractive way of achieving this is by inducing an immune response against the cells of interest, by recruiting immune effector cells such as natural killer (NK) cells, monocytes/macrophages or cytotoxic T lymphocytes (CTLs) to attack and destroy tumor cells.

One way to induce immune effector cell mediated killing or depletion of target cells is via antibody dependent cellular cytoxicity (ADCC) or antibody dependent cellular cytoxicity (ADCP) via ADCC-competent antibodies of the IgG1 isotype as well as antibodies with enhanced ADCC effector function (Zahavi et al, AntibodyTherapeutics, 1, 7-12 (2018)). Alternatively T cells can be recruited for the killing of target cells via (T cell) bispecific antibodies designed to bind to a surface antigen on target cells, and with a second binding moiety to an activating, invariant component of the T cell receptor (TCR) complex (Clynes and Desjarlais, Annu Rev Med 70:427-450 (2019)). Several bispecific formats including BiTE (bispecific T cell engager) (Nagorsen and Bauerle, Exp Cell Res 317, 1255-1260 (2011)) diabodies (Holliger et al., Prot Eng 9, 299-305 (1996)), DART (dual affinity retargeting) (Moore et al., Blood 117, 4542-51 (2011)) or so-called 2+1 T cell bispecific antibodies (TCB) (Bacac et al., Clin Cancer Res 24, 4785-4797 (2018)) have been developed and their suitability for T cell mediated immunotherapy is investigated.

The variety of formats that are being developed shows the great potential attributed to immune cell re-direction and activation in immunotherapy. So far, developed bispecific antibodies always directly engage with the desired antigen of interest, thereby linking target cell and CTL resulting in target cell lysis. Those bispecific antibody formats face challenges related to toxicity, applicability, and producibility. Furthermore, for each single target (combination) individual molecules specific for each target need to be generated. The therapeutic utility of antibodies and their derivatives are not limited to function as T cell engagers but also find indication in modulation of inhibitory or activatory checkpoints. Exemplary, using immune checkpoint inhibiting antibodies showed durable responses in several indications (Hodie et al. N Engl J Med.; 363(8):711-23. (2010); Prieto P A, et al. Clin cancer Res.; 18:2039-2047 (2012)). Furthermore, more recently, it has been shown that the activity of T cell bispecific antibodies can be further enhanced by bispecific agents activating so-called costimulatory pathways on T cells via activation of CD28 (Skokos et al., Sci Trans Med 12(525):1-14 (2020)) or 4-1BB signaling (Claus et al., Sci Trans Med 11(496), eaav5989 (2019)).

Besides current successes, therapies are limited in their flexibility to target multiple different antigens and also in their capacity to selectively exploit NK or CTL function combined in one application.

### SUMMARY OF THE INVENTION

Provided herein is an immune activating fragment crystallizable (Fc) domain binding molecule comprising
(a) an Fc domain binding moiety that specifically binds to a target Fc domain comprising a first set of at least one amino acid substitution, and
(b) an immune activating moiety.

In one embodiment, the first set of at least one amino acid substitution reduce binding to an Fc receptor and/or reduce effector function.

In one embodiment, the immune activating Fc domain binding molecule further comprising
(c) a half-life extending Fc domain,
wherein the Fc domain binding moiety does not specifically bind to the half-life extending Fc domain.

In one embodiment, the half-life extending Fc domain comprises a second set of at least one amino

In one embodiment, the second set of at least one amino acid substitution reduce binding to an Fc

In one embodiment, the target Fc domain and/or the half-life extending Fc domain is composed of a first and a second subunit capable of stable association.

In one embodiment, the target Fc domain and/or the half-life extending Fc domain is an IgG Fc domain, specifically an IgG₁ or IgG₄ Fc domain.

In one embodiment, the target Fc domain exhibits reduced binding affinity to an Fc receptor and/or reduced effector function, as compared to a native IgG₁ Fc domain.

In one embodiment, the half-life extending Fc domain exhibits reduced binding affinity to an Fc receptor and/or reduced effector function, as compared to a native IgG₁ Fc domain.

In one embodiment, the first set of at least one amino acid substitution reduces binding affinity to an Fc receptor and/or effector function, and wherein the second set of at least one amino acid substitution comprises one or more amino acid substitutions at the same amino acid positions as in the first set of at least one amino acid substitution, wherein the amino acids in the second set of at least one amino acid substitution are substituted with different amino acids at the same positions compared to the first set of at least one amino acid substitution.

In one embodiment, the second set of at least one amino acid substitution reduce binding affinity to an Fc receptor and/or effector function.

In one embodiment, the first set of at least one amino acid substitution comprises at least one amino acid substitution at a position selected from the list consisting of 233, 234, 235, 238, 253, 265, 269, 270, 297, 310, 331, 327, 329 and 435 (numberings according to Kabat EU index).

In one embodiment, the second set of at least one amino acid substitution comprises at least one amino acid substitution at a position selected from the list consisting of 233, 234, 235, 238, 253, 265, 269, 270, 297, 310, 331, 327, 329 and 435 (numberings according to Kabat EU index).

In one embodiment, the first set of at least one amino acid substitution comprises the amino acid substitution P329G (numbering according to Kabat EU index) and wherein the second set of at least one amino acid substitution comprises a substitution at position P329 by an amino acid other than glycine (G) (numbering according to Kabat EU index).

In one embodiment, the second set of at least one amino acid substitution comprises a substitution at position P329 (numbering according to Kabat EU index) by an amino acid selected from the list consisting of arginine (R), leucine (L), isoleucine (I), and alanine (A).

In one embodiment, the second set of at least one amino acid substitution comprises a substitution at position P329 (numbering according to Kabat EU index) by arginine (R).

### BRIEF DESCRIPTION OF THE DRAWINGS

FIG. 1. Illustration of the concept of the present invention. A targeting antibody comprising at least one antigen binding moiety capable of specific binding to a target cell is combined with an immune activating Fc domain binding molecule to generate a versatile set of off-the shelf molecules for human therapy. The targeting antibody comprises at least one amino acid substitution in its Fc domain (herein referred to as the target Fc domain) and the immune activating Fc domain binding molecule is capable of specific binding to an Fc domain comprising such amino acid substitution(s) (hereinafter referred to as the first set of at least one amino acid substitution). The immune activating Fc domain binding molecule is capable of specific binding to the targeting antibody (comprising the first set of at least one amino acid substitution) via an antigen binding moiety herein after referred to as the Fc domain binding moiety. The immune activating Fc domain binding molecule further comprises an immune activating moiety (such as e.g. an antigen binding moiety capable of specific binding to CD3, CD28 or 4-1BB) and/or e.g. a cytokine (such as e.g. IL2) and/or a costimulatory ligand (such as e.g. 4-1BBL). The immune activating Fc domain binding molecule is capable of activating an immune cell (e.g., a T cell) via this immune activating moiety. The immune activating Fc domain binding molecule may also comprise an Fc domain, such Fc domain is hereinafter referred to as the half-life extending Fc domain (to discriminate from the target Fc domain). The half-life extending Fc domain may also comprise at least one amino acid substitution (e.g. to decrease effector function), such amino acid substitution(s) are hereinafter referred to as the second set of at least one amino acid substitution (to discriminate from the first set of at least one amino acid substitution). To avoid binding of the immune activating Fc domain binding molecule to other (identical) immune activating Fc domain binding molecules, the Fc domain binding moiety is no capable of specific binding to the half-life extending Fc domain. With this concept, an antibody therapy invoking immune cells can be specifically adapted in terms of required effector functions and/or over time during a treatment. FIG. 43 shows an illustration of an exemplary therapeutic toolbox provided hereinafter.

FIG. 2A to FIG. 2Z. Exemplary configurations of the (multispecific) antibodies of the invention. (FIG. 2A, FIG. 2D) Illustration of the “1+1 CrossMab” molecule. (FIG. 2B, FIG. 2E) Illustration of the “2+1 IgG Crossfab” molecule with alternative order of Crossfab and Fab components (“inverted”). (FIG. 2C, FIG. 2F) Illustration of the “2+1 IgG Crossfab” molecule. (FIG. 2G, FIG. 2K) Illustration of the “1+1 IgG Crossfab” molecule with alternative order of Crossfab and Fab components (“inverted”). (FIG. 2H, FIG. 2L) Illustration of the “1+1 IgG Crossfab” molecule. (FIG. 2I, FIG. 2M) Illustration of the “2+1 IgG Crossfab” molecule with two CrossFabs. (FIG. 2J, FIG. 2N) Illustration of the “2+1 IgG Crossfab” molecule with two CrossFabs and alternative order of Crossfab and Fab components (“inverted”). (FIG. 2O, FIG. 2S) Illustration of the “Fab-Crossfab” molecule. (FIG. 2P, FIG. 2T) Illustration of the “Crossfab-Fab” molecule. (FIG. 2Q, FIG. 2U) Illustration of the “(Fab)₂-Crossfab” molecule. (FIG. 2R, FIG. 2V) Illustration of the “Crossfab-(Fab)₂” molecule. (FIG. 2W, FIG. 2Y) Illustration of the “Fab-(Crossfab)₂” molecule. (FIG. 2X, FIG. 2Z) Illustration of the “(Crossfab)₂-Fab” molecule. ++, --: amino acids of opposite charges optionally introduced in the CH1 and CL domains. Crossfab molecules are depicted as comprising an exchange of VH and VL regions, but may—in aspects wherein no charge modifications are introduced in CH1 and CL domains—alternatively comprise an exchange of the CH1 and CL domains.

FIG. 3A to FIG. 3I. Binding of huIgG1 P329x variants to captured recombinant human Fcg receptors. (FIG. 3A) Setup; recombinant FcgR is captured by an anti-His antibody immobilized on the chip surface. In a second step, huIgG1 P329x variants at a concentration of 150, 300 and 600 nM are injected and interaction with FcgR analysed. (FIG. 3B) Sensorgram showing the binding of huIgG1 P329x variants to huFcgRIa. (FIG. 3C) Sensorgram showing the binding of huIgG1 P329x variants to huFcgRIIa. (FIG. 3D) Sensorgram showing the binding of huIgG1 P329x variants to huFcgRIIb. (FIG. 3E) Sensorgram showing the binding of huIgG1 P329x variants to huFcgRIIIa. (FIG. 3E) Sensorgram showing the binding of huIgG1 P329x variants to huFcgRIIa. (FIG. 3F) Sensorgram showing the binding of huIgG1 P329x variants to huFcgRIIb. (FIG. 3G) Sensorgram showing the binding of huIgG1 P329x variants to huFcgRIIb. (FIG. 3H) Sensorgram showing the binding of huIgG1 P329x variants to huFcgRIIIa. (FIG. 3I) Sensorgram showing the binding of huIgG1 P329x variants to huFcgRIIIa.

FIG. 4A to FIG. 4E. Binding of huIgG1 P329x LALA variants to anti P329G antibody. (FIG. 4A) Setup of the assay; anti-P329G(M-1.7.24) antibody was coupled to the surface of the sensor chip. In a second step, the huIgG1 P329x variants were injected at a concentration of 500 nM (done in triplicates). HuIgG1 P329G was used as positive control. (FIG. 4B) Sensorgram showing the interaction of huIgG1 P329L to anti-P329G(M-1.7.24) antibody (triplicates). (FIG. 4C) Sensorgram showing the interaction of huIgG1 P329I to anti-P329G(M-1.7.24) antibody (done in triplicates). (FIG. 4D) Sensorgram showing the interaction of huIgG1 P329R to anti-P329G(M-1.7.24) antibody (done in triplicates). (FIG. 4E) Sensorgram showing the interaction of huIgG1 P329A to anti-P329G(M-1.7.24) antibody (done in triplicates).

FIG. 5A to FIG. 5E. (FIG. 5A) Schematic illustration of the T-cell bispecific antibody (TCB) molecules used in the Examples. All tested TCB antibody molecules were produced as “2+1 IgG CrossFab, inverted” with charge modifications (VH/VL exchange in CD3 binder, charge modifications in target cell antigen binders, EE=147E, 213E; RK=123R, 124K). FIG. 5B to 5E Components for the assembly of the TCB: light chain of anti-TYRP1 Fab molecule with charge modifications in CH1 and CL (FIG. 5B), light chain of anti-CD3 crossover Fab molecule (FIG. 5C), heavy chain with knob and PG LALA mutations in Fc region (FIG. 5D), heavy chain with hole and PG LALA mutations in Fc region (FIG. 5E).

FIG. 6. Schematic illustration of the surface plasmon resonance (SPR) setup used in Example 3. Anti-PG antibody coupled to a C1 sensorchip. Human and cynomolgus CD3 (fused to an Fc region) are passed over the surface to analyze the interaction of the anti-CD3 antibody in the TCB with CD3.

FIG. 7A to FIG. 7B. The TCBs containing optimized anti-CD3 antibodies were tested in a Jurkat NFAT reporter assay with CHO-K1 TYRP1 clone 76 as target cells. Comparison was done to a TCB containing CD3_{orig}. Activation of Jurkat NFAT reporter cells was determined by measuring luminescence after 4 hours (FIG. 7A) and 24 hours (FIG. 7B) upon treatment.

FIG. 8A to FIG. 8B. Tumor cell killing of the melanoma cell line M150543 with PBMCs from a healthy donor was assessed when treated with TCBs either containing the optimized anti-CD3 antibodies or the parental binder CD3_{orig}. Tumor cell killing was measured by quantification of LDH release after 24 hours (FIG. 8A) and 48 hours (FIG. 8B).

FIG. 9A to FIG. 9D. CD25 and CD69 upregulation on CD8 T cells (FIG. 9A, FIG. 9B) and on CD4 T cells (FIG. 9C, FIG. 9D) was analyzed for PBMCs from a healthy donor treated with TCBs either containing the optimized anti-CD3 antibodies or the parental binder CD3_{orig}, in presence of the M150543 melanoma cell line as target cells. Analysis was done by flow cytometry after 48 hours.

FIG. 10A to FIG. 10B. CD25 expression on CD8 (FIG. 10A) and on CD4 T cells (FIG. 10B) was analyzed for PBMCs from a healthy donor treated with TCBs either containing the optimized anti-CD3 antibodies or the parental binder CD3_{orig}, in absence of tumor target cells. Analysis was done by flow cytometry after 48 hours.

FIG. 11A to FIG. 11D. (FIG. 11A) Schematic illustration of the monovalent IgG molecules generated in Example 19. The monovalent IgG molecules were produced as human IgG₁ with a VH/VL exchange in the CD3 binder. (FIG. 11B-FIG. 11D) Components for the assembly of the monovalent IgG: light chain of anti-CD3 crossover Fab molecule (FIG. 11B), heavy chain with knob and PG LALA mutations in Fc region (FIG. 11C), heavy chain with hole and PG LALA mutations in Fc region (FIG. 11D).

FIG. 12A to FIG. 12B. (FIG. 12A) Exemplary configurations of T cell activating bispecific antigen binding molecules (TCBs) of the invention. Illustration of the anti-P329G×CD3 1+1 universal TCB (uTCB). (FIG. 12B) Exemplary configuration of the binding mode of 1+1 uTCB to the P329G mutation of a tumor targeting IgG and the T cell receptor (TCR) on a T cell. ++, −−: amino acids of opposite charges introduced in the CH and CL domains.

FIG. 13A to FIG. 13B. (FIG. 13A) Exemplary configurations of T cell activating bispecific antigen binding molecules (TCBs) of the invention. Illustration of the anti-P329G×CD3 2+1 universal TCB (uTCB). (FIG. 13B) Exemplary configuration of the binding mode of 2+1 uTCB to the P329G mutation of a tumor targeting IgG and the T cell receptor (TCR) on a T cell. The 2+1 uTCB format is capable of binding two tumor targeting antibodies possessing the P32G mutation simultaneously. ++, −−: amino acids of opposite charges introduced in the CH and CL domains.

FIG. 14A to FIG. 14D depict schematics of different immune activating Fc binding molecules with an anti-CD3 effector moiety (other effector moieties can be used in the same format, i.e., replace the anti-CD3 effector moiety, e.g., anti-CD28, anti-4-1BB). The half-life extending Fc domain comprises a P329x mutation wherein x is an amino acid other than glycine (G). FIG. 14A: 1+1 format, anti-P329G, crossed anti-CD3, charge variants KK/EE, P329x, LALA, knob/hole. FIG. 14B: Classical 2+1 format, anti-P329G, crossed anti-CD3, charge, P329x, LALA, knob/hole. FIG. 14C to FIG. 14D: Inverted 2+1 format, anti-P329G, crossed anti-CD3, charge, P329x, LALA, knob/hole.

FIG. 15A to FIG. 15C. FIG. 15A) Anti-P329G (VH3VL1)×CD3 (CH2527) 1+1 TCB can bind to immobilized human CD3 epsilon-delta-Fc and to hu Fc (P329G) at the same time; FIG. 15B) Anti-P329G (VH3VL1)×CD3 (P035.093) 1+1 TCB can bind to immobilized human CD3 epsilon-delta-Fc and to hu Fe (P329G) at the same time; FIG. 15C) Anti-P329G (VH3VL1)×CD3 (P035.093) 2+1 TCB can bind to immobilized human CD3 epsilon-delta-Fc and to hu Fe (P329G) at the same time. Triplicate injection.

FIG. 16A to FIG. 16E. Kinetic activation of T cells by different concentrations anti-P329G (M-1.7.24)×CD3 (CH2527) 2+1 TCB in combination with different concentrations of anti-FolR1 (6D5) P329G LALA huIgG1 antibodies. Assessed by quantification of the intensity of CD3 downstream signalling using Jurkat-NFAT reporter assay. Depicted are technical average values from triplicates, error bars indicate SD

FIG. 17A to FIG. 17E: Kinetic activation of T cells by different concentrations anti-P329G (M-1.7.24)×CD3 (CH2527) 2+1 TCB in combination with different concentrations of anti-CD20 (GA101) P329G LALA huIgG1 antibodies. Assessed by quantification of the intensity of CD3 downstream signalling using Jurkat-NFAT reporter assay. Depicted are technical average values from triplicates, error bars indicate SD.

FIG. 18A to FIG. 18E. Kinetic activation of T cells by different concentrations of anti-P329G (M-1.7.24)×CD3 (CH2527) 2+1 TCB in combination with different concentrations of anti-FAP (4B9) P329G LALA huIgG1 antibodies. Assessed by quantification of the intensity of CD3 downstream signalling using Jurkat-NFAT reporter assay. Depicted are technical average values from triplicates, error bars indicate SD.

FIG. 19A to FIG. 19B. Activation of T cells by varying concentrations of anti-P329G (M-1.7.24)×CD3 (CH2527) 2+1 TCB in combination with anti-CD20 (GA101) P329G LALA huIgG1 antibodies. As target cells either CD20⁺ z-138 (FIG. 6A) or CD20+SU-DHL-4 cells were used. Assessed by quantification of the intensity of CD3 downstream signalling using Jurkat-NFAT reporter assay. Depicted are technical average values from triplicates, error bars indicate SD.

FIG. 20. Specific, dose-dependent activation of T cells in the presence of the tumor targeting anti-CD20 (GA101) antibody with P329G mutation in combination with anti-P329G (M-1.7.24)×CD3 (CH2527) 2+1 TCB. The anti-CD20 wildtype huIgG1 or anti-CD20 LALA mutated huIgG1 do not activate the T cells. Assessed by quantification of the intensity of CD3 downstream signalling using Jurkat-NFAT reporter assay. Depicted are technical average values from triplicates, error bars indicate SD.

FIG. 21A to FIG. 21C. Reduction of target cell count of adherent tumor cells in the presence of anti-P329G (M-1.7.24)×CD3 (CH2527) 2+1 TCB in combination with tumor targeting anti-EpCAM (FIG. 21A), anti-STEAP (FIG. 21B) or anti-FAP (4B9) (FIG. 21C) P329G LALA huIgG1. Assessed by quantification of red nuclear cell counts over time. Depicted are technical average values from triplicates, error bars indicate SD.

FIG. 22A to FIG. 22B. Activation of T cells by different uTCB formats. 1+1 uTCB or 2+1 uTCB with murine or humanized P329G binder and different CD3 binder. Assessed by quantification of the intensity of CD3 downstream signalling using Jurkat-NFAT reporter assay. As target cells either FolR1+HeLa cells (FIG. 22A) or CD19+SU-DHL-4 cells (FIG. 22B) were used. Depicted are technical average values from triplicates, error bars indicate SD.

FIG. 23A to FIG. 23C. FolR1+HeLa target cell lysis using human PBMCs and uTCB in 1+1 uTCB or 2+1 uTCB with humanized P329G binder (E:T ratio 5:1). Ratio of uTCB and P329G LALA IgG1 was 1:2. Tumor cell lysis was assessed after 5.5 h, 20 h and 42 h by calorimetric quantification of lactate dehydrogenase (LDH) release. Depicted are technical average values from triplicates, error bars indicate SD.

FIG. 24A to FIG. 24C. CD19+ Nalm 6 target cell lysis using human PBMCs and uTCB in 1+1 uTCB or 2+1 uTCB with humanized P329G binder (E:T ratio 5:1) and CD3 binder P035.093. Ratio of uTCB and P329G LALA IgG1 was 1:2. Tumor cell lysis was assessed after 5.5 h, 20 h and 42 h by calorimetric quantification of lactate dehydrogenase (LDH) release. Depicted are technical average values from triplicates, error bars indicate SD.

FIG. 25A to FIG. 25E. Illustration immune activating Fc binding molecules comprising anti-PG and anti-CD28 moieties.

FIG. 26. Immobilized anti-P329G (M-1.7.24)×CD28 (TGN1412_var15_crossed) 1+1 can bind to human IgG (P329G) and to human CD28-Fc at the same time. Duplicate injection.

FIG. 27. Binding analysis of bispecific antigen binding molecules to human CD28 overexpressed on CHO transfectant cells. Depicted are relative median fluorescence valus (MFI) from triplicates with SD. EC50 value of binding was calculated by GraphPadPrism.

FIG. 28. IL2-reporter cell assay after 4 hours of incubation, as determined by luminescence. 25 000 IL2-reporter effector cells were incubated with a fixed concentration of 625 pM of a CD3 IgG (PGLALA-containing Fc) in the presence or absence of increasing concentrations of PG-CD28 (8.4 pM-34.4 nM). As a control PG-CD28 was included in the presence of an isotype control (with PGLALA-containing Fc), respective a tumor-targeting CD28 molecule that is not crosslinked in this assay set-up due to absence of tumor targets. Relative luminescence (RLUs) was determined as direct measurement of Jurkat activation after 4 h. Depicted are RLU values from triplicates with SD.

FIG. 29A to FIG. 29D. (FIG. 29A) depict a schematic of an immune activating Fc binding molecules with an IL2v (cytokine) effector moiety (FIG. 29B) Anti-P329G (M-1.7.24)×IL2v hugG1 can bind to immobilized huIL2R-Fc and hu Fc (P329) at the same time. Triplicate injection (FIG. 29C) IL-2 signaling (STAT5-P) depicted as frequency of STAT5-P in human PD1+ CD4 T cells upon 12 min exposure to IL-2v based molecules. Mean±SEM of 2 donors. (FIG. 29D) IL-2 signaling (STAT5-P) depicted as MFI of STAT5-P in human PD1+CD4 T cells upon 12 min exposure to IL-2v based molecules. Mean±SEM of 2 donors.

FIG. 30A to FIG. 30B. Components for the assembly of monovalent P329G targeted split trimeric human 4-1BB ligand. (FIG. 30A) dimeric ligand fused to human IgG1-CL domain. (FIG. 30B) monomeric ligand fused to human IgG1-CH1 domain.

FIG. 31. Monovalent P329G-targeted split trimeric 4-1BB ligand Fc (kih) LALA fusion containing CH-CL cross with charged residues, also termed anti-P329G×4-1BBL huIgG1.* charged residues FIG. 32A to FIG. 32B. Simultaneous binding of anti-P329G (M-1.7.24)×4-1BBL huIgG1 to hu4-1BB and huIgG1-P329G. (FIG. 32A) setup; (FIG. 32B) Simultaneous binding of anti-P329G(M-1.7.24)×4-1BBL huIgG1 to hu4-1BB-Fc(kih) and human IgG1 containing P329G mutation in the Fc. Duplicates are shown.

FIG. 33A to FIG. 33B. B cell-depleted PBMCs were incubated with WSU DLCL2 for 3 days in the presence of glofitamab (CD20-TCB, 1 nM), anti-P329G×4-1BBL (1 nM) or the combination of both. Tumor cell lysis was determined by LDH release (left) and T cell activation by flow cytometry (right, example: CD4+ T cells, day 3, median fluorescence intensity).

FIG. 34. The bispecific antigen binding molecule is in huIgG1 LALA format comprising two anti-4-1BB Fab fragments (bivalent binding to 4-1BB) and one anti-P329G cross-Fab fragment (a Fab fragment, wherein the VH and VL region are exchanged) which is fused at the C-terminus of its heavy chain to the N-terminus of the heavy chain of one of the 4-1BB Fab fragments. This format is termed herein the 2+1 format. The big black dot symbolizes the knob-into-hole mutations, whereas the small black dots in the CH1/CL domains symbolize amino acid mutation that improve the correct pairing of the heavy chains with the anti-4-1BB light chains.

FIG. 35A to FIG. 35B. Different assays set ups were compared with each other. The anti-P329G(M-1.7.24)×4-1BBL huIgG1 molecule was tested for its functionality using a Jurkat reporter cell line assay. Therefore tumor target (Her2, CEACAM5, FAP) expressing cells (KPL4, MKN45, NIH/3T3-huFAP clone 19) were coincubated with human 4-1BB receptor expressing Jurkat reporter cells (Jurkat-hu4-1BB-NFkB-luc2) and different concentrations of tumor target (TT)-specific human IgG1 P329G LALA antibodies in the presence or absence of anti-P329G(M-1.7.24)×4-1BBL huIgG1 for 5 hours. Afterwards Luciferase activity was measured by adding a detection solution (One-Glo) and measuring the light emission released during luciferase-mediated oxidation (FIG. 35A). This activity was directly compared with directly tumor targeted TT-4×1BBL huIgG1 as a positive control (FIG. 35B).

FIG. 36A to FIG. 36B. Testing of different ratios between anti-P329G(M-1.7.24)×4-1BBL huIgG1 and tumor-target specific huIgG1 P329G LALA. The anti-P329G(M-1.7.24)×4-1BBL huIgG1 molecule was tested for its functionality using a Jurkat reporter cell line assay, whereby molecules were either kept in solution or crosslinked by the addition of Her2+KPL4 human breast cancer cells FIG. 36A. Direct tumor-targeted Her2×4-1BBL huIgG1 was compared with indirect crosslinked anti-P329G(M-1.7.24)×4-1BBL huIgG1. Hereby anti-Her2 huIgG1 P329G LALA served as linker between the tumor target Her2 and anti-P329G(M-1.7.24)×4-1BBL huIgG1, whereby the ratio between anti-Her2 huIgG1 P329G LALA and anti-P329G(M-1.7.24)×4-1BBL huIgG1 was kept stable (FIG. 6A). The same set up was also tested with CEACAM5+MKN45 gastric cancer cells and CEACAM5-specific antibodies FIG. 36B.

FIG. 37A to FIG. 37C. The anti-P329G(M-1.7.24)×4-1BBL huIgG1 molecule was tested for its functionality using a Jurkat reporter cell line assay, whereby molecules were either kept in solution or crosslinked by the addition of Her2+KPL4 human breast cancer cells FIG. 37A. Direct tumor-targeted Her2×4-1BBL huIgG1 was compared with indirect crosslinked anti-P329G(M-1.7.24)×4-1BBL huIgG1 which was linked by a anti-Her2-specific huIgG1 P329G LALA given in a ratio 1:2 was kept stable. Further non-binding (DP47) molecules were included as controls. The same was repeated with CEACAM5+MKN45 gastric cancer cells FIG. 37B and FAP+NIH/3T3-huFAP clone 19 fibroblast cells FIG. 37C.

FIG. 38A to FIG. 38B (FIG. 38A) Exemplary Illustration of an ADCC competent IgG1 effector molecule able to bind to the P329G mutation (anti-P329G IgG1) of a tumor targeting molecule (e.g. IgG1, SM). (FIG. 38B) Exemplary configuration of the binding mode of the anti-P329G IgG1 effector molecule to the P329G mutation of a tumor targeting IgG and the FcγIII on immune effector cells.

FIG. 39. Antibody-dependent cellular cytotoxicity (ADCC) mediated by anti-P329G (VH3VL1) huIgG1 with glycoengineered Fc (GE) in the presence of tumor targeting IgG1 with P329G LALA mutation. Assessed by quantification of the lactate dehydrogenase (LDH) release of target cells. Depicted is the average of technical triplicates, error bars indicate SD. As statistical analysis a one-way ANOVA analysis with Bonferroni-correction was performed. As p value the New England Journal of Medicine style was used as listed in GraphPadPrism 7. Meaning *=P≤0,033; **=P≤0,002; ***=P<0.001.

FIG. 40. Downregulation of CD16 receptor on NK cells upon activation with the anti-P329G (VH3VL1) huIgG1 in combination with the tumor targeting IgG1 with P329G LALA mutation. Assessed by flow cytometry. Depicted is the average values from technical triplicates, error bars indicate SD. As statistical analysis a one-way ANOVA analysis with Bonferroni-correction was performed. As p value the New England Journal of Medicine style was used as listed in GraphPadPrism 7. Meaning *=P≤0,033; **=P≤0,002; ***=P≤0.001. FIG. 41. Upregulation of CD107a on NK cells upon activation with the anti-P329G (VH3VL1) huIgG1 in combination with the tumor targeting IgG1 with P329G LALA mutation. Assessed by flow cytometry. Depicted is the average values from technical triplicates, error bars indicate SD. As statistical analysis a one-way ANOVA analysis with Bonferroni-correction was performed. As p value the New England Journal of Medicine style was used as listed in GraphPadPrism 7. Meaning *=P≤0,033; **=P≤0,002; ***=P≤0.001.

FIG. 42A to FIG. 42B. Only the combination of anti-FAP (clone 4B9) human IgG1 P329GLALA and anti-P329G human IgG1 mAb induces dose dependent NFAT activation in Jurkat FcγRIIIa reporter cells, which is a measure of ADCC competency. Each point represents the mean value of technical duplicates of one experiment. Standard error of the mean is indicated by error bars. (FIG. 42A) A fixed concentration (10 pg/mL) of anti-FAP (4B9) P329G LALA huIgG1 was used in combination with an 8-fold decreasing serial titration of the anti-P329G huIgG1 mAB. (FIG. 42B) An 8 fold decreasing serial titration of anti-FAP (4B9) P329G LALA huIgG1 was used in combination with a fixed concentration (10 pg/mL) of the anti-P329G huIgG1. The anti-P329G huIgG1 was tested as fully fucosylated (triangle) and afucosylated (circle) human IgG1 isotype.

FIG. 43. Illustration of an exemplary therapeutic toolbox provided hereinafter. A (therapeutic) targeting antibody capable of specific binding to a target cell is combined with different immune activating Fc domain binding moieties capable of specific binding to the P329G mutation in the Fc domain of the targeting antibody. The provided effector functions include a glycoengineered Fc domain (e.g., ADCC), anti-CD3 (e.g., T cell activation), 4-1BBL and/or anti-4-1BB (e.g., T cell costimulation), anti-CD28 (e.g., T cell costimulation) and IL2v (e.g. T cell proliferation). The effector functions can be titrated to optimal concentrations in combination and/or over time to maximize therapeutic benefit.

FIG. 44. Illustration of an exemplary configuration for cis-targeting of PD-1 positive T cell. A targeting antibody capable of specific binding to PD1 and comprising the P329G mutation is combined with an immune activating Fc domain binding molecule comprising an IL2v immune activating moiety.

FIG. 45A to FIG. 45C. Kinetic activation of T cells by different concentrations of anti-FOLR1 P329G LALA huIgG1 with anti-P329G (VH3VL1)×CD3 (P035.093) 2+1 TCB, P329R LALA Fc (molar ratio IgG:TCB 2:1). Concentration of the TCBs used: 0 nM (FIG. 45A), 0.05 nM (FIG. 45B), 5 nM (FIG. 45C). HeLa (FOLR1+) cells were used as target cells. Assessed by quantification of the intensity of CD3 downstream signaling using Jurkat-NFAT reporter assay. Depicted are technical average values from triplicates; error bars indicate SD.

FIG. 46A to FIG. 46D. Activation of T cells by anti-FOLR1 P329G LALA huIgG1 with anti-P329G (VH3VL1)×CD3 (P035.093) 2+1 TCB LALA Fc (molar ratio IgG:TCB 2:1) on several FOLR1+ target cell lines. As target cells, HeLa (FIG. 46A), JAR (FIG. 46B), OVCAR-3 (FIG. 46C), SKOV-3 (FIG. 46D) were used. HeLa (FOLR1+) cells were used as target cells. Assessed by quantification of the intensity of CD3 downstream signaling using Jurkat-NFAT reporter assay. Depicted are technical average values from triplicates; error bars indicate SD.

FIG. 47. Activation of T cells by anti-FOLR1 P329G LALA huIgG1 with anti-P329G (VH3VL1)×CD3 (P035.093) 2+1 TCB containing LALA Fc or P329R LALA Fc (molar ratio IgG:TCB 2:1). HeLa (FOLR1+) cells were used as target cells. Assessed by quantification of the intensity of CD3 downstream signaling using Jurkat-NFAT reporter assay. Depicted are technical average values from triplicates; error bars indicate SD.

FIG. 48A to FIG. 48D. Primary human T cell activation measured by CD25 upregulation on CD8+ T cells, in presence of anti-FOLR1 P329G LALA huIgG1 with anti-P329G (VH3VL1)×CD3 (P035.093) 2+1 TCB containing either LALA Fc or P329R LALA Fc (molar ratio IgG:TCB 2:1). As effector cells, either pan T cells (FIG. 48A, FIG. 48C) or PBMCs (FIG. 48B, FIG. 48D) from a healthy donor were used. Either SKOV-3 (FOLR1+) (FIG. 48A, FIG. 48B) or no target cells (FIG. 48C, FIG. 48D) were used. Analysis was done by flow cytometry after 48 h. Depicted are technical average values from triplicates; error bars indicate SD.

FIG. 49A to FIG. 49E. Activation of T cells by tumor-targeting P329G LALA huIgG1 with anti-P329G (VH3VL1)×CD3 2+1 TCB P329R LALA Fc (molar ratio IgG:TCB 2:1), with P035.093, CH2527 or Clone 22 as a CD3 binder. Performed on several targets and several target cells. As target and target cell pairs, the following were used: CD19+ SU-DHL-8 cells (FIG. 49A), FOLR1+ HeLa cells (FIG. 49B), CEA+MKN-45 cells (FIG. 49C), HER2+ LNCaP cells (FIG. 49D), STEAP1+ LNCaP cells (FIG. 49E). Assessed by quantification of the intensity of CD3 downstream signaling using Jurkat-NFAT reporter assay. Depicted are technical average values from triplicates; error bars indicate SD.

FIG. 50A to FIG. 50B. Kinetics of tumor cell lysis by primary human pan T cells in presence of anti-FOLR1 (FIG. 50A) or anti-CEA (FIG. 50B) P329G LALA huIgG1 with anti-P329G (VH3VL1)×CD3 (P035.093) 2+1 TCB P329R LALA Fc. As target cells, HeLa NLR (FOLR1+) (FIG. 50A) and MKN-45 NLR (FIG. 50B) were used. Assessed by quantification of red nuclear cell counts over time. Depicted are technical average values from triplicates; error bars indicate SD.

FIG. 51. Kinetics of tumor cell lysis by primary human pan T cells in presence of anti-FOLR1 P329G LALA huIgG1 with anti-P329G (VH3VL1)×CD3 2+1 TCB P329R LALA Fc (molar ratio IgG:TCB 2:1), with P035.093, CH2527 or Clone 22 as a CD3 binder. As target cells, HeLa NLR (FOLR1+) were used. Assessed by quantification of red nuclear cell counts over time. Depicted are technical average values from triplicates; error bars indicate SD.

FIG. 52A to FIG. 52C. Primary human T cell activation measured by CD69 upregulation on CD8+ T cells, in presence of anti-FOLR1 P329G LALA huIgG1 with anti-P329G (VH3VL1)×CD3 2+1 TCB P329R LALA Fc (molar ratio IgG:TCB 2:1), with P035.093, CH2527 or Clone 22 as a CD3 binder. As effector cells, pan T cells from three healthy donors were used—donor A (FIG. 52A), donor B (FIG. 52B), donor C (FIG. 52C). HeLa (FOLR1+) were used as target cells. Analysis was done by flow cytometry after 48 h. Depicted are technical average values from triplicates; error bars indicate SD.

FIG. 53. Activation of 4-1BB reporter T cells by costimulatory molecules anti-P329G (VH3VL1)×4-1BBL LALA huIgG1, 1+1 and anti-P329G (VH3VL1)×CD28 LALA huIgG1, 1+1, in presence of 100 nM anti-CEA P329G LALA huIgG1 and 0.5 nM anti-P329G (VH3VL1)×CD3 (P035.093) 2+1 TCB P329R LALA Fc. SKOV-3 huCEA (CEA+) cells were used as target cells. Assessed by quantification of the intensity of 4-1BB downstream signaling using Jurkat-NFxB reporter assay. Depicted are technical average values from triplicates; error bars indicate SD.

### DETAILED DESCRIPTION OF THE INVENTION

### Definitions

Terms are used herein as generally used in the art, unless otherwise defined in the following.

As used herein, the term “antigen binding molecule” refers in its broadest sense to a molecule that specifically binds an antigenic determinant. Examples of antigen binding molecules are immunoglobulins and derivatives, e.g. fragments, thereof.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence. The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

An “activating T cell antigen” as used herein refers to an antigenic determinant expressed on the surface of a T lymphocyte, particularly a cytotoxic T lymphocyte, which is capable of inducing T cell activation upon interaction with an antigen binding molecule. Specifically, interaction of an antigen binding molecule with an activating T cell antigen may induce T cell activation by triggering the signaling cascade of the T cell receptor complex. In a particular embodiment the activating T cell antigen is CD3, particularly the epsilon subunit of CD3 (see UniProt no. P07766 (version 130), NCBI RefSeq no. NP_000724.1; or UniProt no. Q95LI5 (version 49), NCBI GenBank no. BAB71849.1).

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary methods for measuring binding affinity are described in the following.

An “affinity matured” antibody refers to an antibody with one or more alterations in one or more complementary determining regions (CDRs), compared to a parent antibody which does not possess such alterations, such alterations resulting in an improvement in the affinity of the antibody for antigen.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Particular amino acid mutations are amino acid substitutions. For the purpose of altering e.g. the binding characteristics of an Fc region, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “antigen binding domain” refers to the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable domain (VL) and an antibody heavy chain variable domain (VH).

An “antigen binding site” refers to the site, i.e. one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

As used herein, the term “antigen binding moiety” refers to a polypeptide molecule that specifically binds to an antigenic determinant. In one embodiment, an antigen binding moiety is able to direct the entity to which it is attached (e.g. a second antigen binding moiety) to a target site, for example to a specific type of tumor cell or tumor stroma bearing the antigenic determinant. In another embodiment an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain embodiments, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

As used herein, the term “antigenic determinant” is synonymous with “antigen” and “epitope” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

“Antibody-dependent cell-mediated cytotoxicity” (“ADCC”) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG₁ isotype. In certain aspects, the antibody is of the IgG₁ isotype with the P329G, L234A and L235A mutation to reduce Fc-region effector function. In other aspects, the antibody is of the IgG₂ isotype. In certain aspects, the antibody is of the IgG₄ isotype with the S228P mutation in the hinge region to improve stability of IgG4 antibody. The heavy chain constant domains that correspond to the different classes of immunoglobulins are called α, δ, ε, γ, and μ, respectively. The light chain of an antibody may be assigned to one of two types, called kappa (x) and lambda (k), based on the amino acid sequence of its constant domain.

The terms “constant region derived from human origin” or “human constant region” as used in the current application denotes a constant heavy chain region of a human antibody of the subclass IgG1, IgG2, IgG3, or IgG4 and/or a constant light chain kappa or lambda region. Such constant regions can be used in human or humanized antibodies and are well known in the state of the art and e.g. described by Kabat, E. A., et al., Sequences of Proteins of Immunological Interest, 5th ed., Public Health Service, National Institutes of Health, Bethesda, MD (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, MD (1991), NIH Publication 91-3242.

By a “crossover” Fab molecule (also termed “Crossfab”) is meant a Fab molecule wherein the variable domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the crossover Fab molecule.

An “effective amount” of an agent, e.g., a pharmaceutical composition, refers to an amount effective, at dosages and for periods of time necessary, to achieve the desired therapeutic or prophylactic result.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

As used herein, the terms “first”, “second” or “third” with respect to Fab molecules etc., are used for convenience of distinguishing when there is more than one of each type of moiety. Use of these terms is not intended to confer a specific order or orientation of the immune activating Fc domain binding molecule unless explicitly so stated.

A “Fab molecule” refers to a protein consisting of the VH and CH1 domain of the heavy chain (the “Fab heavy chain”) and the VL and CL domain of the light chain (the “Fab light chain”) of an immunoglobulin.

By “fused” is meant that the components (e.g. a Fab molecule and an Fc domain subunit) are linked by peptide bonds, either directly or via one or more peptide linkers.

As used herein, the term “single-chain” refers to a molecule comprising amino acid monomers linearly linked by peptide bonds. In certain embodiments, one of the antigen binding moieties is a single-chain Fab molecule, i.e. a Fab molecule wherein the Fab light chain and the Fab heavy chain are connected by a peptide linker to form a single peptide chain. In a particular such embodiment, the C-terminus of the Fab light chain is connected to the N-terminus of the Fab heavy chain in the single-chain Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

The terms “full length antibody”, “intact antibody”, and “whole antibody” are used herein interchangeably to refer to an antibody having a structure substantially similar to a native antibody structure or having heavy chains that contain an Fc region as defined herein.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain (also referred to herein as a “cleaved variant heavy chain”). This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Amino acid sequences of heavy chains including Fc domains (or a subunit of an Fc domain as defined herein) are denoted herein without C-terminal glycine-lysine dipeptide if not indicated otherwise. In one embodiment of the invention, a heavy chain including a subunit of an Fc domain as specified herein, comprises an additional C-terminal glycine-lysine dipeptide (G446 and K447, numbering according to EU index of Kabat). In one embodiment of the invention, a heavy chain including a subunit of an Fc domain as specified herein, comprises an additional C-terminal glycine residue (G446, numbering according to EU index of Kabat). Compositions of the invention, such as the pharmaceutical compositions described herein, comprise a population of antigen binding molecules of the invention. The population of antigen binding molecule may comprise molecules having a full-length heavy chain and molecules having a cleaved variant heavy chain. The population of antigen binding molecules may consist of a mixture of molecules having a full-length heavy chain and molecules having a cleaved variant heavy chain, wherein at least 50%, at least 60%, at least 70%, at least 80% or at least 90% of the antigen binding molecules have a cleaved variant heavy chain. In one embodiment of the invention a composition comprising a population of antigen binding molecules of the invention comprises an antigen binding molecule comprising a heavy chain including a subunit of an Fc domain as specified herein with an additional C-terminal glycine-lysine dipeptide (G446 and K447, numbering according to EU index of Kabat). In one embodiment of the invention a composition comprising a population of antigen binding molecules of the invention comprises an immune activating Fc domain binding molecule comprising a heavy chain including a subunit of an Fc domain as specified herein with an additional C-terminal glycine residue (G446, numbering according to EU index of Kabat). In one embodiment of the invention such a composition comprises a population of antigen binding molecules comprised of molecules comprising a heavy chain including a subunit of an Fc domain as specified herein; molecules comprising a heavy chain including a subunit of a Fc domain as specified herein with an additional C-terminal glycine residue (G446, numbering according to EU index of Kabat); and molecules comprising a heavy chain including a subunit of an Fc domain as specified herein with an additional C-terminal glycine-lysine dipeptide (G446 and K447, numbering according to EU index of Kabat). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

An “Fc domain binding moiety” as herein used is an antigen binding moiety capable of binding to an Fc domain.

A “half-life extending Fe” as herein used is the Fc domain (where present) comprised in the immune activating Fc domain binding molecule of the invention. A “target Fe” as herein used is the Fc domain comprised in a targeting antibody of the invention.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

An “activating Fe receptor” is an Fe receptor that following engagement by an Fc domain of an antibody elicits signaling events that stimulate the receptor-bearing cell to perform effector functions. Human activating Fe receptors include FcγRIIIa (CD16a), FcγRI (CD64), FcγRIIa (CD32), and FeαRI (CD89).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al., supra. In one aspect, for the VH, the subgroup is subgroup III as in Kabat et al., supra. A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)). Unless otherwise indicated, the CDRs are determined according to Kabat et al., supra. One of skill in the art will understand that the CDR designations can also be determined according to Chothia, supra, McCallum, supra, or any other scientifically accepted nomenclature system.

An “immune activating moiety” as used herein refers to one or more polypeptide(s) inducing activation of an immune cell (e.g. a T cell) upon interaction with an antigen, receptor or ligand (or other elements of the cells inducing activation) on the immune cell. An example of an immune activating moiety is antigen binding molecule capable of binding to an activating T cell antigen triggering the signaling cascade of the T cell receptor complex. In a particular embodiment the immune activating moiety is an antigen binding moiety capable of binding to CD3, particularly the epsilon subunit of CD3 (see UniProt no. P07766 (version 130), NCBI RefSeq no. NP_000724.1; or UniProt no. Q95LI5 (version 49), NCBI GenBank no. BAB71849.1). Other exemplary immune activating moieties are cytokines (e.g. IL2), antigen binding moieties capable of binding to a costimulatory T cell antigen (e.g. CD28, 4-1BB) or costimulatory ligands (e.g. 4-1BBL) as described herein.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ(IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler ert al, Nature Medicine 2017, published online 12 Jun. 2017, doi:10.1038/nm.4356 or EP 2 101 823 B1).

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header. As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

The term “package insert” is used to refer to instructions customarily included in commercial packages of therapeutic products, that contain information about the indications, usage, dosage, administration, combination therapy, contraindications and/or warnings concerning the use of such therapeutic products.

“Reduced binding”, for example reduced binding to an Fc receptor, refers to a decrease in affinity for the respective interaction, as measured for example by SPR. For clarity the term includes also reduction of the affinity to zero (or below the detection limit of the analytic method), i.e. complete abolishment of the interaction. Conversely, “increased binding” refers to an increase in binding affinity for the respective interaction.

By “specific binding” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) of ≤1 μM, ≤100 nM, ≤nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The immune activating Fc domain binding molecules of the invention are capable of inducing T cell activation. Suitable assays to measure T cell activation are known in the art described herein.

A “target cell antigen” as used herein refers to an antigenic determinant presented on the surface of a target cell, for example a cell in a tumor such as a cancer cell or a cell of the tumor stroma. In a particular embodiment, the target cell antigen is CD20, particularly human CD20 (see UniProt no. P11836).

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

The term “valent” as used herein denotes the presence of a specified number of antigen binding sites in an antigen binding molecule. As such, the term “monovalent binding to an antigen” denotes the presence of one (and not more than one) antigen binding site specific for the antigen in the antigen binding molecule.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The term “interleukin-2” or “IL-2” as used herein, refers to any native IL-2 from any vertebrate source, including mammals such as primates (e.g. humans) and rodents (e.g., mice and rats), unless otherwise indicated. The term encompasses unprocessed IL-2 as well as any form of IL-2 that results from processing in the cell. The term also encompasses naturally occurring variants of IL-2, e.g. splice variants or allelic variants. The amino acid sequence of an exemplary human IL-2 is shown in SEQ ID NO: 166. Unprocessed human IL-2 comprises an N-terminal 20 amino acid signal peptide, which is absent in the mature IL-2 molecule.

The term “IL-2 mutant” or “mutant IL-2 polypeptide” as used herein is intended to encompass any mutant forms of various forms of the IL-2 molecule including full-length IL-2, truncated forms of IL-2 and forms where IL-2 is linked to another molecule such as by fusion or chemical conjugation. “Full-length” when used in reference to IL-2 is intended to mean the mature, natural length IL-2 molecule. For example, full-length human IL-2 refers to a molecule that has 133 amino acids (see e.g. SEQ ID NO: 166). The various forms of IL-2 mutants are characterized in having a at least one amino acid mutation affecting the interaction of IL-2 with CD25. This mutation may involve substitution, deletion, truncation or modification of the wild-type amino acid residue normally located at that position. Mutants obtained by amino acid substitution are preferred. Unless otherwise indicated, an IL-2 mutant may be referred to herein as a mutant IL-2 peptide sequence, a mutant IL-2 polypeptide, a mutant IL-2 protein or a mutant IL-2 analog.

Designation of various forms of IL-2 is herein made with respect to the sequence shown in SEQ ID NO: 19. Various designations may be used herein to indicate the same mutation. For example a mutation from phenylalanine at position 42 to alanine can be indicated as 42A, A42, A₄₂, F42A, or Phe42Ala.

By a “human IL-2 molecule” as used herein is meant an IL-2 molecule comprising an amino acid sequence that is at least about 90%, at least about 91%, at least about 92%, at least about 93%, at least about 94%, at least about 95% or at least about 96% identical to the human IL-2 sequence of SEQ ID NO:166. Particularly, the sequence identity is at least about 95%, more particularly at least about 96%. In particular embodiments, the human IL-2 molecule is a full-length IL-2 molecule.

The term “CD25” or “α-subunit of the IL-2 receptor” as used herein, refers to any native CD25 from any vertebrate source, including mammals such as primates (e.g. humans) and rodents (e.g., mice and rats), unless otherwise indicated. The term encompasses “full-length”, unprocessed CD25 as well as any form of CD25 that results from processing in the cell. The term also encompasses naturally occurring variants of CD25, e.g. splice variants or allelic variants. In certain embodiments CD25 is human CD25. The amino acid sequence of human CD25 is found e.g. in UniProt entry no. P01589 (version 185).

The term “high-affinity IL-2 receptor” as used herein refers to the heterotrimeric form of the IL-2 receptor, consisting of the receptor γ-subunit (also known as common cytokine receptor γ-subunit, γ_{c}, or CD132, see UniProt entry no. P14784 (version 192)), the receptor β-subunit (also known as CD122 or p70, see UniProt entry no. P31785 (version 197)) and the receptor α-subunit (also known as CD25 or p55, see UniProt entry no. P01589 (version 185)). The term “intermediate-affinity IL-2 receptor” by contrast refers to the IL-2 receptor including only the γ-subunit and the β-subunit, without the α-subunit (for a review see e.g. Olejniczak and Kasprzak, Med Sci Monit 14, RA179-189 (2008)).

The term “TNF ligand family member” or “TNF family ligand” refers to a proinflammatory cytokine. Cytokines in general, and in particular the members of the TNF ligand family, play a crucial role in the stimulation and coordination of the immune system. At present, nineteen cyctokines have been identified as members of the TNF (tumour necrosis factor) ligand superfamily on the basis of sequence, functional, and structural similarities. All these ligands are type II transmembrane proteins with a C-terminal extracellular domain (ectodomain), N-terminal intracellular domain and a single transmembrane domain. The C-terminal extracellular domain, known as TNF homology domain (THD), has 20-30% amino acid identity between the superfamily members and is responsible for binding to the receptor. The TNF ectodomain is also responsible for the TNF ligands to form trimeric complexes that are recognized by their specific receptors. Members of the TNF ligand family are selected from the group consisting of Lymphotoxin a (also known as LTA or TNFSF1), TNF (also known as TNFSF2), LTβ (also known as TNFSF3), OX40L (also known as TNFSF4), CD40L (also known as CD154 or TNFSF5), FasL (also known as CD95L, CD178 or TNFSF6), CD27L (also known as CD70 or TNFSF7), CD30L (also known as CD153 or TNFSF8), 4-1BBL (also known as TNFSF9), TRAIL (also known as APO2L, CD253 or TNFSF10), RANKL (also known as CD254 or TNFSF11), TWEAK (also known as TNFSF12), APRIL (also known as CD256 or TNFSF13), BAFF (also known as CD257 or TNFSF13B), LIGHT (also known as CD258 or TNFSF14), TL1A (also known as VEGI or TNFSF15), GITRL (also known as TNFSF18), EDA-A1 (also known as ectodysplasin A1) and EDA-A2 (also known as ectodysplasin A2). The term refers to any native TNF family ligand from any vertebrate source, including mammals such as primates (e.g. humans), non-human primates (e.g. cynomolgus monkeys) and rodents (e.g. mice and rats), unless otherwise indicated.

The term “costimulatory TNF ligand family member” or “costimulatory TNF family ligand” refers to a subgroup of TNF ligand family members, which are able to costimulate proliferation and cytokine production of T-cells. These TNF family ligands can costimulate TCR signals upon interaction with their corresponding TNF receptors and the interaction with their receptors leads to recruitment of TNFR-associated factors (TRAF), which initiate signalling cascades that result in T-cell activation. Costimulatory TNF family ligands are selected from the group consisting of 4-1BBL, OX40L, GITRL, CD70, CD30L and LIGHT, more particularly the costimulatory TNF ligand family member is 4-1BBL.

As described herein before, 4-1BBL is a type II transmembrane protein and one member of the TNF ligand family. Complete or full length 4-1BBL having the amino acid sequence of SEQ ID NO:69 has been described to form trimers on the surface of cells. The formation of trimers is enabled by specific motives of the ectodomain of 4-1BBL. Said motives are designated herein as “trimerization region”. The amino acids 50-254 of the human 4-1BBL sequence form the extracellular domain of 4-1BBL, but even fragments thereof are able to form the trimers. In specific embodiments of the invention, the term “ectodomain of 4-1BBL or a fragment thereof” refers to a polypeptide having an amino acid sequence selected from SEQ ID NO:120 (amino acids 52-254 of human 4-1BBL), SEQ ID NO:117 (amino acids 71-254 of human 4-1BBL), SEQ ID NO:119 (amino acids 80-254 of human 4-1BBL) and SEQ ID NO:118 (amino acids 85-254 of human 4-1BBL) or a polypeptide having an amino acid sequence selected from SEQ ID NO:121 (amino acids 71-248 of human 4-1BBL), SEQ ID NO:124 (amino acids 52-248 of human 4-1BBL), SEQ ID NO:123 (amino acids 80-248 of human 4-1BBL) and SEQ ID NO:122 (amino acids 85-248 of human 4-1BBL), but also other fragments of the ectodomain capable of trimerization are included herein.

An “ectodomain” is the domain of a membrane protein that extends into the extracellular space (i.e. the space outside the target cell). Ectodomains are usually the parts of proteins that initiate contact with surfaces, which leads to signal transduction. The ectodomain of TNF ligand family member as defined herein thus refers to the part of the TNF ligand protein that extends into the extracellular space (the extracellular domain), but also includes shorter parts or fragments thereof that are responsible for the trimerization and for the binding to the corresponding TNF receptor. The term “ectodomain of a TNF ligand family member or a fragment thereof” thus refers to the extracellular domain of the TNF ligand family member that forms the extracellular domain or to parts thereof that are still able to bind to the receptor (receptor binding domain).

As used herein, the term “PD1”, “human PD1”, “PD-1” or “human PD-1” (also known as Programmed cell death protein 1, or Programmed Death 1) refers to the human protein PD1. See also UniProt entry no. Q15116 (version 156). As used herein, an antibody “binding to PD-1”, “specifically binding to PD-1”, “that binds to PD-1” or “anti-PD-1 antibody” refers to an antibody that is capable of binding PD-1, especially a PD-1 polypeptide expressed on a cell surface, with sufficient affinity such that the antibody is useful as a diagnostic and/or therapeutic agent in targeting PD-1. In one embodiment, the extent of binding of an anti-PD-1 antibody to an unrelated, non-PD-1 protein is less than about 10% of the binding of the antibody to PD-1 as measured, e.g., by radioimmunoassay (RIA) or flow cytometry (FACS) or by a Surface Plasmon Resonance assay using a biosensor system such as a Biacore® system. In certain embodiments, an antibody that binds to PD-1 has a KD value of the binding affinity for binding to human PD-1 of ≤1 μM, ≤100 nM, ≤10 nM, 1 nM, ≤0.1 nM, ≤0.01 nM, or ≤0.001 nM (e.g. 10⁸ M or less, e.g. from 10⁸ M to 10⁻¹³ M, e.g., from 10⁻⁹ M to 10⁻¹³ M). In one embodiment, the KD value of the binding affinity is determined in a Surface Plasmon Resonance assay using the Extracellular domain (ECD) of human PD-1 as antigen.

### DETAILED DESCRIPTION OF THE INVENTION

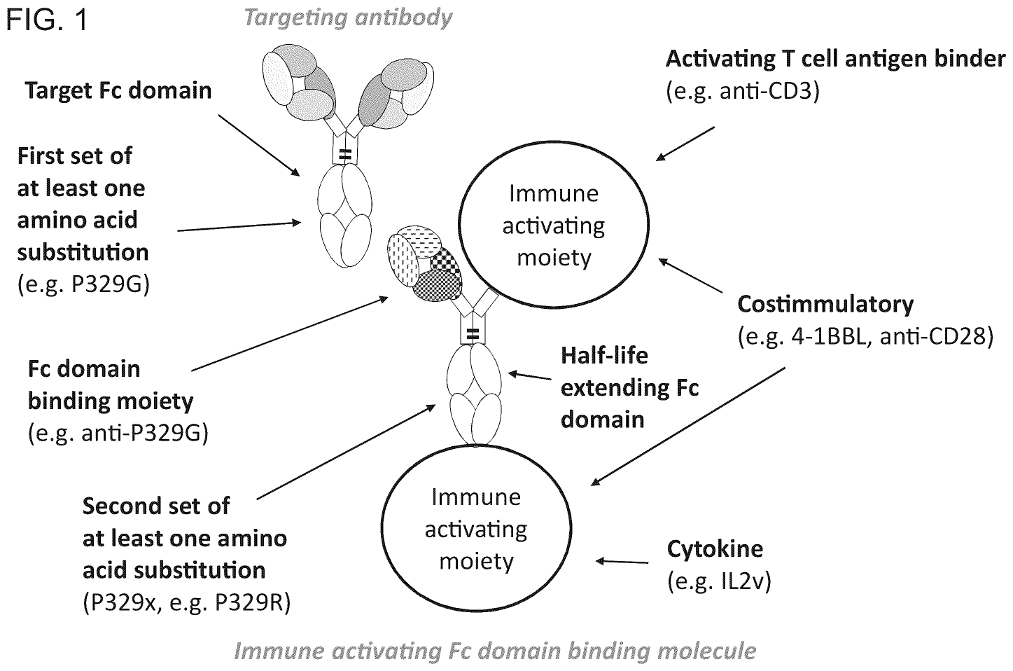

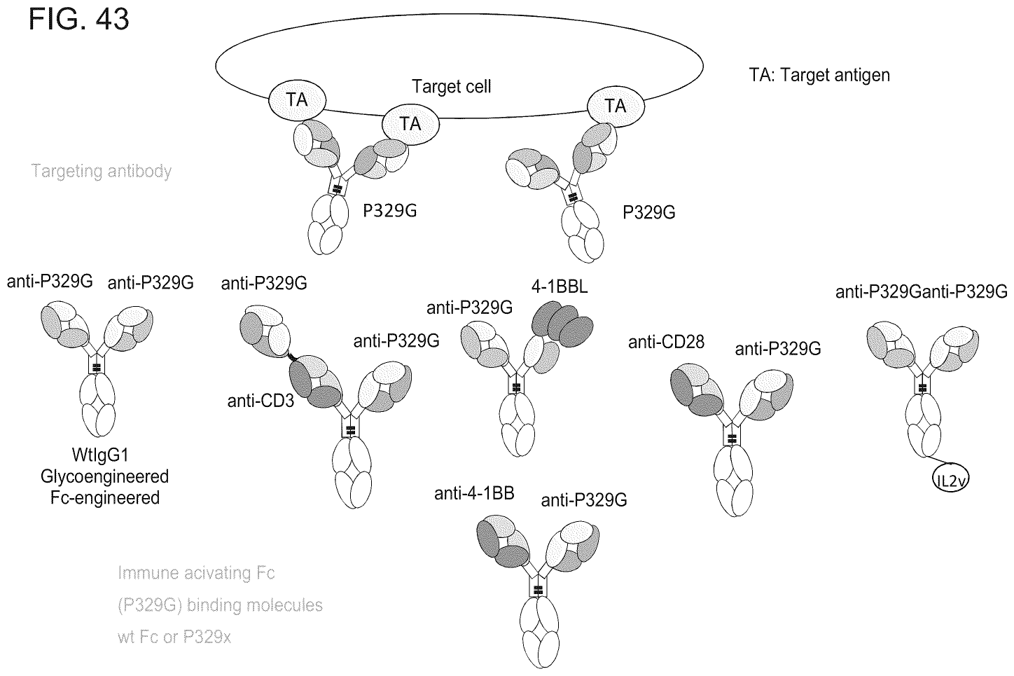

The present invention provides a modular antibody based platform for flexible antigen targeting and individual immune cell stimulation that can be adapted to desired indications. Compared to the conventional bispecific formats and checkpoint modulators that directly engage with their target of interest, the present invention consists of two components that can be individually adapted and used in a plug and play manner. This modular platform mainly focuses on two parts: (i) a targeting antibody for precise and selective antigen targeting via an easy to produce targeting molecule which, possesses the ability to stimulate immune cells if desired and (ii) an immune activating (Fc domain binding) molecule that specifically recognizes the Fc-part of the targeting antibody, thereby recruiting immune effector cells and activating them e.g. via establishing a immunological synapse to redirect CTLs and initiates subsequent lysis of the target cell (see FIG. 1 and FIG. 43). Through the combination of the targeting antibody and the immune activating (Fc domain binding) molecule(s) an individualized, customizable off-the-shelf approach to stimulate individual immune cells is possible without the need to generate different effector molecules for each and every unique surface antigen.

Accordingly, in one aspect, the invention provides an immune activating fragment crystallizable (Fc) domain binding molecule.

In one aspect of the invention, provided is an immune activating fragment crystallizable (Fc) domain binding molecule comprising
(a) an Fc domain binding moiety, and
(b) an immune activating moiety.

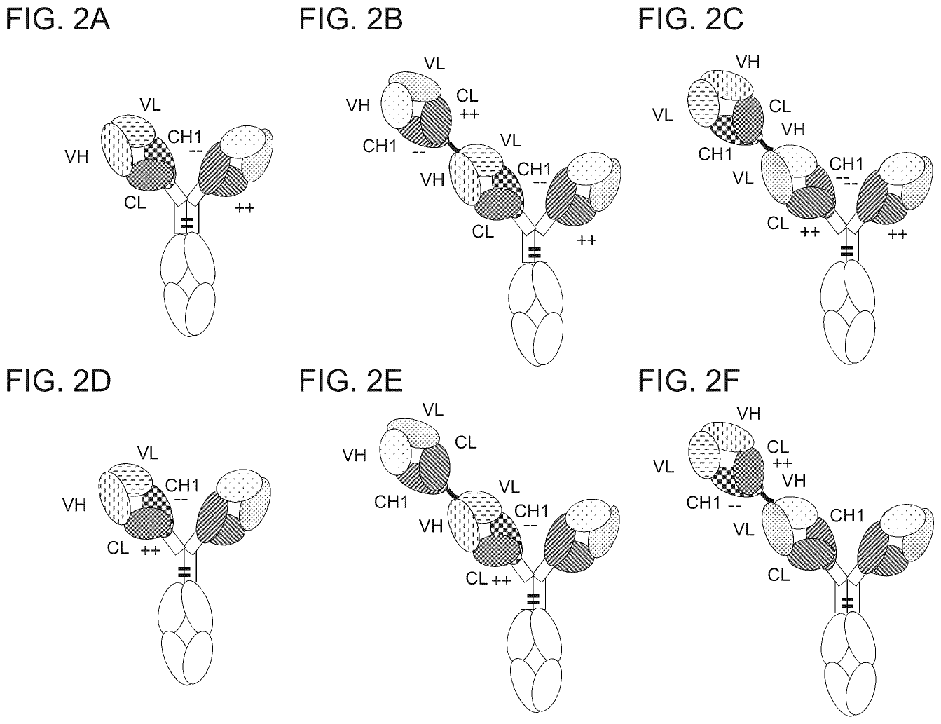

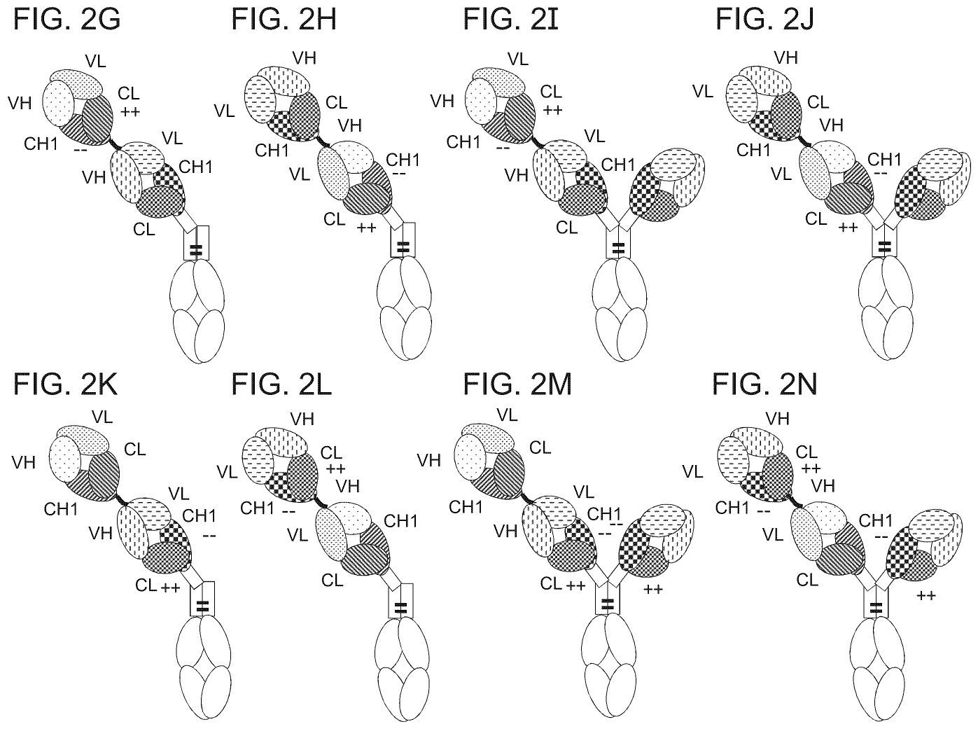

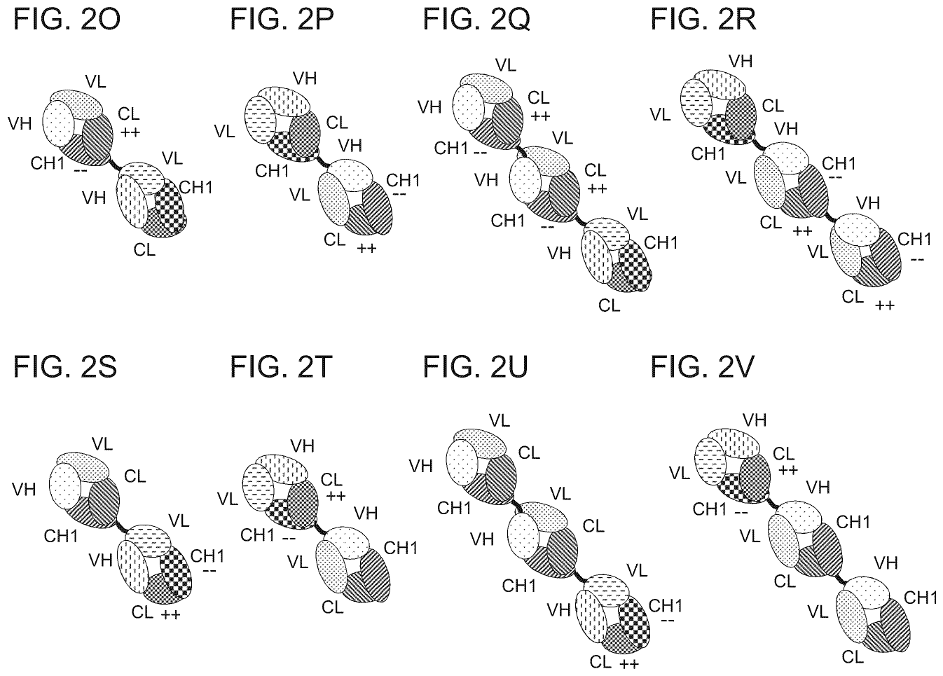

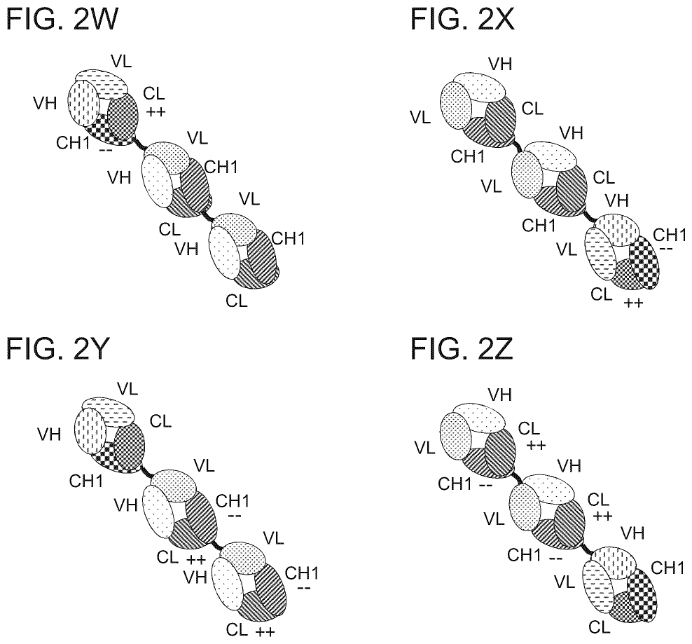

In some aspect, the immune activating Fc domain binding molecule does not comprise an Fc domain for example if a short half-life of the immune acrivating Fc domain binding molecule is preferred. Accordingly, the present invention provides immune activating Fc domain binding molecules devoid of an Fc domain (for illustrative formats see FIG. 2O-2Z).

However, in many instances it will be preferred to include an Fc domain in the immune activating Fc domain binding molecules of the present invention. The Fc domain confers to the antibodies favorable pharmacokinetic properties, including a long serum half-life which contributes to good accumulation in the target tissue and a favorable tissue-blood distribution ratio.

Accordingly, in a preferred aspect of the invention, provided is an immune activating fragment crystallizable (Fc) domain binding molecule comprising (c) a half-life extending Fc domain. Notably, it may be desirable to ensure that the Fc domain binding moiety is not capable of binding to the half-life extending Fc. Binding of the Fc domain binding moiety to the half-life extending Fc domain can lead to self-binding of the immune activating Fc domain binding molecules, i.e. one immune activating Fc domain binding molecules binds to another (identical) Fc domain binding molecule via the half-life extending Fc domain. Self-binding can lead to cross-linking of multiple immune activating Fc domain binding molecules, which can be undesirable.

Accordingly, in a preferred aspect of the present invention, provided an immune activating fragment crystallizable (Fc) domain binding molecule comprising
(a) an Fc domain binding moiety which specifically binds to a target Fc domain comprising a first set of at least one amino acid substitution,
(b) an immune activating moiety, and
(c) a half-life extending Fc,
wherein the Fc domain binding moiety does not specifically bind to the half-life extending Fc domain.

Since the Fc domain binding moiety does not specifically bind to the half-life extending Fc domain, no self-binding or cross-linking will occur, i.e. the immune activating Fc domain binding molecule will (only) recognice and bind to Fc domain comprising a first set of at least one amino acid substitution. The Fc domain comprising a first set of at least one amino acid substitution is herein referred to as target Fc domain. The Fc domain comprises in the immune activating Fc domain binding molecule is herein referred to as half-life extending Fc domain. Without being bound to theory, it will be clear to the skilled person that the target Fc domain can also extend the half life of the targeting antibody. However, to cleary distinguish between the target Fc domain and the Fc domain comprised in the immune activating Fc domain binding molecules of the present invention, the half-life extending Fc domain as herein described will always refer to the Fc domain comprised in the immune activating Fc domain binding molecules.

An Fc domain as herein described (e.g., target Fc domain or half-life extending Fc domain) consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. In one embodiment the immune activating Fc domain binding molecule of the invention comprises not more than one Fc domain.

As herein before described, the Fc domain confers to an antibody favorable pharmacokinetic properties, including a long serum half-life. At the same time it may, however, lead to undesirable targeting to cells expressing Fe receptors rather than to the preferred antigen-bearing cells. Moreover, the co-activation of Fe receptor signaling pathways may lead to cytokine release which, in combination with the T cell activating properties and the long half-life of the immune activating Fc domain binding molecule, results in excessive activation of cytokine receptors and severe side effects upon systemic administration. Activation of (Fe receptor-bearing) immune cells other than T cells may even reduce efficacy of the immune activating Fc domain binding molecule due to the potential destruction of T cells e.g. by NK cells.

As herein described, in preferred embodiments, the target Fc domain comprise a first set of at least one amino acid substitution. In one embodiment, the first set of at least one amino acid substitution reduce binding to an Fe receptor and/or reduce effector function. Furthermore, in embodiments wherein the immune activating Fc domain binding molecule comprises a half-life extending Fe domain, the half-life extending Fc domain may comprise a second set of at least one amino acid substitution. In one embodiment, the second set of at least one amino acid substitution reduce binding to an Fe receptor and/or reduce effector function.

Hence, one particular aspect of the present invention is to reduce effector function of the targeting antibody and/or the immune activating antibody. In such embodiment, the Fc domain binding moiety specifically bind to a Fc domain comprising the first set of at least one amino acid substitution (the target Fc domain) but does not specifically bind to the Fc domain comprising the second set of at least one amino acid substitution (the half-life extending Fc domain). Fc domain binding moieties with such desirable specificity are herein below described and methods to generate further Fc domain binding moieties with the desired specificity are also herein below described (e.g. immunization of a mammalian immune system with an Fc domain comprising the first set of at least one amino acid substitution and screening for Fc domain binding moieties which do not specifically bind to an Fc domain comprising the second set of at least one amino acid substitution wherein the first and/or second set of at least one amino acid substitution decrease binding to an Fc receptor and/or decrease effector function).

An exemplary Fc domain binding moiety which specifically binds to a target Fc domain (wherein the first set of at least one amino acid substitutions comprises the P329G substitution) but not to the half-life extending Fc domain (wherein the second set of at least one amino acid substitutions does not comprise the P329G substitution, i.e. is wildtype at the P329 position or comprises an amino acid substitution at position P329 other than glycine) is the anti-P329G (M-1.7.24) huIgG1 binder comprising the CDR sequences of SEQ ID NO: 1, 2, 3, 4, 5 and 6 (numbering according to Kabat EU index) and as further described in WO2017/072210.

Another exemplary Fc domain binding moiety which specifically binds to a target Fc domain but not to the half-life extending Fc domain is the anti-AAA binder comprising the CDR sequences of SEQ ID NO: 168, 169, 170, 171, 172, 173 (numbering according to Kabat EU index) and as further described in WO2017/072210.

It may be preferred that the target Fc domain and/or the half-life extending Fc domain confer an increased effector function to the targeting antibody and or the immune activating Fc domain binding molecule, respectively. Accordingly, in one embodiment, the first set of at least one amino acid substitution increase binding to an Fc receptor and/or increase effector function. In one embodiment, the second set of at least one amino acid substitution increase binding to an Fc receptor and/or increase effector function. Fc domain binding moieties with such desirable specificity can be generated as herein described, e.g. by immunization of a mammalian immune system with an Fc domain comprising the first set of at least one amino acid substitution and screening for Fc domain binding moieties which do not specifically bind to an Fc domain comprising the second set of at least one amino acid substitution wherein the first and/or second set of at least one amino acid substitution increase binding to an Fc receptor and/or increase effector function.

Fc mutations (e.g. amino acid substitutions) conferring such binding to Fc receptors and/or effector function are known in the art and herein below described. In one embodiment, the target Fc domain and/or the half-life extending Fc domain is an IgG Fc domain, specifically an IgG₁ or IgG₄ Fc domain. In one embodiment, the target Fc domain and/or the half-life extending Fc domain exhibit reduced binding affinity to an Fc receptor and/or reduced effector function, as compared to a native IgG₁ Fc domain. In one such embodiment the target Fc domain and/or the half-life extending Fc domain (or the molecules comprising said Fc domains) individually exhibits less than 50%, preferably less than 20%, more preferably less than 10% and most preferably less than 5% of the binding affinity to an Fc receptor, as compared to a native IgG₁ Fc domain (or a molecule comprising a native IgG₁ Fc domain), and/or less than 50%, preferably less than 20%, more preferably less than 10% and most preferably less than 5% of the effector function, as compared to a native IgG₁ Fc domain domain (or a molecule comprising a native IgG₁ Fc domain). In one embodiment, the target Fc domain and/or the half-life extending Fc domain (or molecules comprising said Fc domain) do not substantially bind to an Fc receptor and/or induce effector function. In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of CDC, ADCC, ADCP, and cytokine secretion. In a particular embodiment the effector function is ADCC. In one embodiment the target Fc domain and/or the half-life extending Fc domain individually exhibit substantially similar binding affinity to neonatal Fc receptor (FcRn), as compared to a native IgG₁ Fc domain domain. Substantially similar binding to FcRn is achieved when the target Fc domain and/or the half-life extending Fc domain (or the molecules comprising said Fc domain) individually exhibits greater than about 70%, particularly greater than about 80%, more particularly greater than about 90% of the binding affinity of a native IgG₁ Fc domain (or molecule comprising a native IgG₁ Fc domain) to FcRn.

In certain embodiments the target Fc domain and/or the half-life extending Fc domain are individually engineered to have reduced binding affinity to an Fc receptor and/or reduced effector function, as compared to a non-engineered Fc domain. In particular embodiments, the target Fc domain and/or the half-life extending Fc domain individually comprise one or more amino acid substitution that reduces the binding affinity of the Fc domain to an Fc receptor and/or effector function. Typically, the same one or more amino acid substitution is present in each of the two subunits of the target Fc domain and/or in each of the two subunits the half-life extending Fc domain. However, the amino acid substitutions in the target Fc domain and the amino acid substitutions in the half-life extending Fc domain cannot be identical if non-binding of the Fc domain binding moiety to the half-life extending Fc domain should be ensured. In such embodiments, a first set of at least one amino acid substitution and a second set of at least one amino acid substitution is envisaged as described herein below each individually comprising at least one amino acid substitution that reduces binding to an Fc receptor and/or effector function. In one embodiment the amino acid substitution reduces the binding affinity of an Fc domain to an Fc receptor. In one embodiment the amino acid substitution reduces the binding affinity of an Fc domain to an Fc receptor by at least 2-fold, at least 5-fold, or at least 10-fold. In embodiments where there is more than one amino acid substitution that reduces the binding affinity of the target Fc domain and/or the half-life extending Fc domain to the Fc receptor, the combination of these amino acid substitutions may reduce the binding affinity of the Fc domain to an Fc receptor by at least 10-fold, at least 20-fold, or even at least 50-fold. In one embodiment the targeting antibody and/or the immune activating Fc domain binding molecule individually comprise an engineered Fc domain that exhibits less than 20%, particularly less than 10%, more particularly less than 5% of the binding affinity to an Fc receptor as compared to molecule comprising a non-engineered Fc domain. In a particular embodiment the Fc receptor is an Fcγ receptor. In some embodiments the Fc receptor is a human Fc receptor. In some embodiments the Fc receptor is an activating Fc receptor. In a specific embodiment the Fc receptor is an activating human Fcγ receptor, more specifically human FcγRIIIa, FcγRI or FcγRIIa, most specifically human FcγRIIIa. Preferably, binding to each of these receptors is reduced. In some embodiments binding affinity to a complement component, specifically binding affinity to C1q, is also reduced. In one embodiment binding affinity to neonatal Fc receptor (FcRn) is not reduced. Substantially similar binding to FcRn, i.e. preservation of the binding affinity of the Fc domain to said receptor, is achieved when the Fc domain (or a molecule comprising said Fc domain) exhibits greater than about 70% of the binding affinity of a non-engineered form of the Fc domain (or a molecule comprising said non-engineered form of the Fc domain) to FcRn. The target Fc domain and/or the half-life extending Fc domain, or molecules of the invention comprising said Fc domain, may individually exhibit greater than about 80% and even greater than about 90% of such affinity. In certain embodiments the target Fc domain and/or the half-life extending Fc domain are individually engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one embodiment the reduced effector function is one or more selected from the group of reduced CDC, reduced ADCC, reduced ADCP, and reduced cytokine secretion. In a particular embodiment the reduced effector function is reduced ADCC. In one embodiment the reduced ADCC is less than 20% of the ADCC induced by a non-engineered Fc domain (or a molecule comprising a non-engineered Fc domain).

First Set of at Least One Amino Acid Substitution

The first set of at least one amino acid substitution is included in the targeting antibody (in the target Fc domain) as illustrated in FIG. 1. Accordingly, in one aspect of the present invention, the target Fc domain as herein described comprises a first set of at least one amino acid substitution. In one embodiment, the first set of at least one amino acid substitution comprises at least one amino acid substitution that reduces the binding affinity of the target Fc domain to an Fc receptor and/or effector function. In one embodiment the target Fc domain comprises an amino acid substitution at a position selected from the group of E233, L234, L235, N297, P331 and P329 (numberings according to Kabat EU index). In a more specific embodiment the target Fc domain comprises an amino acid substitution at a position selected from the group of L234, L235 and P329 (numberings according to Kabat EU index). In some embodiments the target Fc domain comprises the amino acid substitutions L234A and L235A (numberings according to Kabat EU index). In one such embodiment, the target Fc domain is an IgG₁ Fc domain, particularly a human IgG₁ Fc domain. In one embodiment the target Fc domain comprises an amino acid substitution at position P329. In a more specific embodiment the amino acid substitution is P329A or P329G, particularly P329G (numberings according to Kabat EU index). In one embodiment the target Fc domain comprises an amino acid substitution at position P329 and a further amino acid substitution at a position selected from E233, L234, L235, N297 and P331 (numberings according to Kabat EU index). In a more specific embodiment the further amino acid substitution is E233P, L234A, L235A, L235E, N297A, N297D or P331S. In particular embodiments the target Fc domain comprises amino acid substitutions at positions P329, L234 and L235 (numberings according to Kabat EU index). In more particular embodiments the target Fc domain comprises the amino acid substitutions L234A, L235A and P329G (“P329G LALA”). In one such embodiment, the target Fc domain is an IgG₁ Fc domain, particularly a human IgG₁ Fc domain. The “P329G LALA” combination of amino acid substitutions almost completely abolishes Fcγ receptor (as well as complement) binding of a human IgG₁ Fc domain, as described in PCT publication no. WO 2012/130831, incorporated herein by reference in its entirety. WO 2012/130831 also describes methods of preparing such mutant Fc domains and methods for determining its properties such as Fc receptor binding or effector functions.

IgG₄ antibodies exhibit reduced binding affinity to Fc receptors and reduced effector functions as compared to IgG₁ antibodies. Hence, in some embodiments the target Fc domain of the targeting antibody is an IgG₄ Fc domain, particularly a human IgG₄ Fc domain. In one embodiment the IgG₄ target Fc domain comprises amino acid substitutions at position S228, specifically the amino acid substitution S228P (numberings according to Kabat EU index). To further reduce its binding affinity to an Fc receptor and/or its effector function, in one embodiment the IgG₄ target Fc domain comprises an amino acid substitution at position L235, specifically the amino acid substitution L235E (numberings according to Kabat EU index). In another embodiment, the IgG₄ target Fc domain comprises an amino acid substitution at position P329, specifically the amino acid substitution P329G (numberings according to Kabat EU index). In a particular embodiment, the IgG₄ target Fc domain comprises amino acid substitutions at positions S228, L235 and P329, specifically amino acid substitutions S228P, L235E and P329G (numberings according to Kabat EU index). Such IgG₄ Fc domain mutants and their Fcγ receptor binding properties are described in PCT publication no. WO 2012/130831, incorporated herein by reference in its entirety.

In a particular embodiment the target Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG₄ Fc domain comprising the amino acid substitutions S228P, L235E and optionally P329G (numberings according to Kabat EU index).

In certain embodiments N-glycosylation of the target Fc domain has been eliminated. In one such embodiment the target Fc domain comprises an amino acid substitution at position N297, particularly an amino acid substitution replacing asparagine by alanine (N297A) or aspartic acid (N297D) (numberings according to Kabat EU index).

In addition to the target Fc domains described hereinabove, target Fc domains with reduced Fc receptor binding and/or effector function also include those with substitution of one or more of Fc domain residues 238, 265, 269, 270, 297, 327 and 329 (U.S. Pat. No. 6,737,056) (numberings according to Kabat EU index). Such target Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Mutant target Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined, e.g. by ELISA, or by Surface Plasmon Resonance (SPR) using standard instrumentation such as a BIAcore instrument (GE Healthcare), and Fc receptors may be obtained by recombinant expression. A suitable such binding assay is described herein. Alternatively, binding affinity of target Fc domains or targeting antibody comprising a target Fc domain for Fc receptors may be evaluated using cell lines known to express particular Fc receptors, such as human NK cells expressing FcγIIIa receptor.

Effector function of the target Fc domain, or a targeting antibody comprising such target Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some embodiments, binding of the target Fc domain to a complement component, specifically to C1q, is reduced. Accordingly, in some embodiments wherein the target Fc domain is engineered to have reduced effector function, said reduced effector function includes reduced CDC. C1q binding assays may be carried out to determine whether the targeting antibody is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

Second Set of at Least One Amino Acid Substitution

The second set of at least one amino acid substitution is included in the half-life extending Fc domain of the immune activating Fc domain binding molecules as illustrated in FIG. 1. Accordingly, in one aspect of the present invention, the half-life extending Fc domain as herein described comprises a second set of at least one amino acid substitution. In one embodiment, the second set of at least one amino acid substitution comprises at least one amino acid substitution that reduces the binding affinity of the half-life extending Fc domain to an Fc receptor and/or effector function. In one embodiment the half-life extending Fc domain comprises an amino acid substitution at a position selected from the group of E233, L234, L235, N297, P331 and P329 (numberings according to Kabat EU index). In a more specific embodiment the half-life extending Fc domain comprises an amino acid substitution at a position selected from the group of L234, L235 and P329 (numberings according to Kabat EU index). In some embodiments the half-life extending Fc domain comprises the amino acid substitutions L234A and L235A (numberings according to Kabat EU index). In one such embodiment, the half-life extending Fc domain is an IgG₁ Fc domain, particularly a human IgG₁ Fc domain. In one embodiment the half-life extending Fc domain comprises an amino acid substitution at position P329. In a more specific embodiment the amino acid substitution is P329A or P329G, particularly P329G (numberings according to Kabat EU index). In one embodiment the half-life extending Fc domain comprises an amino acid substitution at position P329 and a further amino acid substitution at a position selected from E233, L234, L235, N297 and P331 (numberings according to Kabat EU index). In a more specific embodiment the further amino acid substitution is E233P, L234A, L235A, L235E, N297A, N297D or P331S. In particular embodiments the half-life extending Fc domain comprises amino acid substitutions at positions P329, L234 and L235 (numberings according to Kabat EU index).

In a preferred embodiment the half-life extending Fc domain comprises the amino acid substitutions L234A, L235A (“LALA”, numbering according to Kabat EU index). In more particular embodiments the half-life extending Fc domain comprises the amino acid substitutions L234A, L235A and P329G (“P329G LALA”, numbering according to Kabat EU index). In one such embodiment, the half-life extending Fc domain is an IgG₁ Fc domain, particularly a human IgG₁ Fc domain. The “P329G LALA” combination of amino acid substitutions almost completely abolishes Fcγ receptor (as well as complement) binding of a human IgG₁ Fc domain, as described in PCT publication no. WO 2012/130831, incorporated herein by reference in its entirety. WO 2012/130831 also describes methods of preparing such mutant Fc domains and methods for determining its properties such as Fc receptor binding or effector functions.

In a preferred embodiment, the half-life extending Fc domain is an IgG1 and the second set of at least one amino acid substitution comprises the P329G substitution In one particular such embodiment, the half-life extending Fc domain comprises an amino acid sequence that is at least about 95%, 96%, 97%, 98%, 99% or 100% identical to the amino acid sequence of SEQ ID NO: 29.

However, in some embodiment wherein the Fc domain binding moiety is capable of binding to a target Fc domain comprising the P329G substitution, it is preferred that the half-life extending Fc domain comprises an amino acid substitution at position P329 by an amino acid other than glycine (G) (numbering according to Kabat EU index). In one embodiment, the first set of at least one amino acid substitution as herein above described comprises the amino acid substitution P329G (numbering according to Kabat EU index) and the second set of at least one amino acid substitution comprises a substitution at position P329 by an amino acid other than glycine (G) (numbering according to Kabat EU index). In one embodiment, the second set of at least one amino acid substitution comprises a substitution at position P329 (numbering according to Kabat EU index) by an amino acid other than glycine (G) wherein such amino acid is not able to form a proline sandwich between two conserved tryptophan sidechains within a Fc gamma receptor, in particular within FcgRIIIa.

In a preferred such embodiment, the second set of at least one amino acid substitution comprises a substitution at position P329 (numbering according to Kabat EU index) by an amino acid selected from the list consisting of arginine (R), leucine (L), isoleucine (I), and alanine (A). In a more preferred such embodiment, the second set of at least one amino acid substitution comprises a substitution at position P329 (numbering according to Kabat EU index) by arginine (R). The “P329R”, the “P329L”, the “P329I” and the “P329A” amino acid substitutions each individually combined with the “LALA” amino acid substitutions almost completely abolishes Fcγ receptor (as well as complement) as herein described. In one embodiment, the immune activating Fc domain binding molecule comprises a half-life extending Fc domain comprising an amino acid sequence that is at least about 95%, 96%, 97%, 98%, 99% or 100% identical to an amino acid sequence selected from the group consisting of SEQ ID NO: 29, SEQ ID NO: 30, SEQ ID NO: 31, SEQ ID NO: 32, SEQ ID NO: 33.

In a preferred embodiment, the half-life extending Fc domain is an IgG1 and the second set of at least one amino acid substitution comprises the P329L substitution (numbering according to Kabat EU index). In one particular such embodiment, the half-life extending Fc domain comprising the P329L substitution (numbering according to Kabat EU index) comprises an amino acid sequence that is at least about 95%, 96%, 97%, 98%, 99% or 100% identical to the amino acid sequence of SEQ ID NO: 30.

In another preferred embodiment, the half-life extending Fc domain is an IgG1 and the second set of at least one amino acid substitution comprises the P329I substitution (numbering according to Kabat EU index). In one particular such embodiment, the half-life extending Fc domain comprising the P329I substitution (numbering according to Kabat EU index) comprises an amino acid sequence that is at least about 95%, 96%, 97%, 98%, 99% or 100% identical to the amino acid sequence of SEQ ID NO: 31.

In another preferred embodiment, the half-life extending Fc domain is an IgG1 and the second set of at least one amino acid substitution comprises the P329R substitution (numbering according to Kabat EU index). In one particular such embodiment, the half-life extending Fc domain comprising the P329R substitution (numbering according to Kabat EU index) comprises an amino acid sequence that is at least about 95%, 96%, 97%, 98%, 99% or 100% identical to the amino acid sequence of SEQ ID NO: 32.

In another preferred embodiment, the half-life extending Fc domain is an IgG1 and the second set of at least one amino acid substitution comprises the P329A substitution (numbering according to Kabat EU index). In one particular such embodiment, the half-life extending Fc domain comprising the P329A substitution (numbering according to Kabat EU index) comprises an amino acid sequence that is at least about 95%, 96%, 97%, 98%, 99% or 100% identical to the amino acid sequence of SEQ ID NO: 33.

IgG4 antibodies exhibit reduced binding affinity to Fc receptors and reduced effector functions as compared to IgG₁ antibodies. Hence, in some embodiments the half-life extending Fc domain of the immune activating Fc domain binding molecule is an IgG₄ Fc domain, particularly a human IgG4 Fc domain. In one embodiment the IgG4 half-life extending Fc domain comprises amino acid substitutions at position S228, specifically the amino acid substitution S228P (numberings according to Kabat EU index). To further reduce its binding affinity to an Fc receptor and/or its effector function, in one embodiment the IgG4 half-life extending Fc domain comprises an amino acid substitution at position L235, specifically the amino acid substitution L235E (numberings according to Kabat EU index). In another embodiment, the IgG₄ half-life extending Fc domain comprises an amino acid substitution at position P329, specifically the amino acid substitution P329G (numberings according to Kabat EU index). In a particular embodiment, the IgG₄ half-life extending Fc domain comprises amino acid substitutions at positions S228, L235 and P329, specifically amino acid substitutions S228P, L235E and P329G (numberings according to Kabat EU index). Such IgG₄ Fc domain mutants and their Fcγ receptor binding properties are described in PCT publication no. WO 2012/130831, incorporated herein by reference in its entirety.

In a particular embodiment the half-life extending Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG4 Fc domain comprising the amino acid substitutions S228P, L235E and optionally P329G (numberings according to Kabat EU index).

In certain embodiments N-glycosylation of the half-life extending Fc domain has been eliminated.

In one such embodiment the half-life extending Fc domain comprises an amino acid substitution at position N297, particularly an amino acid substitution replacing asparagine by alanine (N297A) or aspartic acid (N297D) (numberings according to Kabat EU index).

In addition to the half-life extending Fc domains described hereinabove, half-life extending Fc domains with reduced Fc receptor binding and/or effector function also include those with substitution of one or more of Fc domain residues 238, 265, 269, 270, 297, 327 and 329 (U.S. Pat. No. 6,737,056) (numberings according to Kabat EU index). Such half-life extending Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Mutant (substituted) half-life extending Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fe receptors can be easily determined, e.g. by ELISA, or by Surface Plasmon Resonance (SPR) using standard instrumentation such as a BIAcore instrument (GE Healthcare), and Fc receptors may be obtained by recombinant expression. A suitable such binding assay is described herein. Alternatively, binding affinity of half-life extending Fc domains or immune activating Fc domain binding molecule comprising a half-life extending Fc domain for Fc receptors may be evaluated using cell lines known to express particular Fc receptors, such as human NK cells expressing FcγIIIa receptor.

Effector function of the half-life extending Fc domain, or an immune activating Fc domain binding molecule comprising such half-life extending Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some embodiments, binding of the half-life extending Fc domain to a complement component, specifically to C1q, is reduced. Accordingly, in some embodiments wherein the half-life extending Fc domain is engineered to have reduced effector function, said reduced effector function includes reduced CDC. C1q binding assays may be carried out to determine whether the immune activating Fc domain binding molecule is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

Fc Domain Modifications Promoting Heterodimerization

The immune activating Fc domain binding molecules according to the invention comprise different Fab molecules and immune activating moieties (e.g., Fab molecules, cytokines, ligands), fused to one or the other of the two subunits of the half-life extending Fc domain, thus the two subunits of the half-life extending Fc domain are typically comprised in two non-identical polypeptide chains. Recombinant co-expression of these polypeptides and subsequent dimerization leads to several possible combinations of the two polypeptides. To improve the yield and purity of immune activating Fc domain binding molecules in recombinant production, it will thus be advantageous to introduce in the Fc domain of the immune activating Fc domain binding molecule (i.e. the half-life extending Fc domain) a modification promoting the association of the desired polypeptides. Accordingly, in particular embodiments the half-life extending Fc domains according to the invention comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

There exist several approaches for modifications in the CH3 domain of the Fc domain in order to enforce heterodimerization, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homdimers between the two first or the two second CH3 domains are formed). These different approaches for improved heavy chain heterodimerization are contemplated as different alternatives in combination with the heavy-light chain modifications (VH and VL exchange/replacement in one binding arm and the introduction of substitutions of charged amino acids with opposite charges in the CH1/CL interface) in the immune activating Fc domain binding molecules according to the invention which reduce light chain mispairing and Bence Jones-type side products.

In a specific embodiment said modification promoting the association of the first and the second subunit of the Fc domain is a so-called “knob-into-hole” modification, comprising a “knob” modification in one of the two subunits of the half-life extending Fc domain and a “hole” modification in the other one of the two subunits of the half-life extending Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular embodiment, in the CH3 domain of the first subunit of the half-life extending Fc domain an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the half-life extending Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W).

Preferably said amino acid residue having a smaller side chain volume is selected from the group consisting of alanine (A), serine (S), threonine (T), and valine (V).

The protuberance and cavity can be made by altering the nucleic acid encoding the polypeptides, e.g. by site-specific mutagenesis, or by peptide synthesis.

In a specific embodiment, in the CH3 domain of the first subunit of the half-life extending Fc domain (the “knobs” subunit) the threonine residue at position 366 is replaced with a tryptophan residue (T366W), and in the CH3 domain of the second subunit of the half-life extending Fc domain (the “hole” subunit) the tyrosine residue at position 407 is replaced with a valine residue (Y407V). In one embodiment, in the second subunit of the half-life extending Fc domain additionally the threonine residue at position 366 is replaced with a serine residue (T366S) and the leucine residue at position 368 is replaced with an alanine residue (L368A) (numberings according to Kabat EU index).

In yet a further embodiment, in the first subunit of the half-life extending Fc domain additionally the serine residue at position 354 is replaced with a cysteine residue (S354C) or the glutamic acid residue at position 356 is replaced with a cysteine residue (E356C), and in the second subunit of the half-life extending Fc domain additionally the tyrosine residue at position 349 is replaced by a cysteine residue (Y349C) (numberings according to Kabat EU index). Introduction of these two cysteine residues results in formation of a disulfide bridge between the two subunits of the Fc domain, further stabilizing the dimer (Carter, J Immunol Methods 248, 7-15 (2001)).

In a particular embodiment, the first subunit of the half-life extending Fc domain comprises amino acid substitutions S354C and T366W, and the second subunit of the half-life extending Fc domain comprises amino acid substitutions Y349C, T366S, L368A and Y407V (numbering according to Kabat EU index).

In a particular embodiment the immune activating moiety is fused to the first subunit of the half-life extending Fc domain (comprising the “knob” modification). Without wishing to be bound by theory, fusion of the immune activating moiety to the knob-containing subunit of the half-life extending Fc domain will (further) minimize the generation of immune activating Fc domain binding molecules comprising two immune activating moieties (steric clash of two knob-containing polypeptides).

Other techniques of CH3-modification for enforcing the heterodimerization are contemplated as alternatives according to the invention and are described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291.

In one embodiment the heterodimerization approach described in EP 1870459 A1, is used alternatively. This approach is based on the introduction of charged amino acids with opposite charges at specific amino acid positions in the CH3/CH3 domain interface between the two subunits of the half-life extending Fc domain. One preferred embodiment for the immune activating Fc domain binding molecules of the invention are amino acid mutations R409D; K370E in one of the two CH3 domains (of the half-life extending Fc domain) and amino acid mutations D399K; E357K in the other one of the CH3 domains of the half-life extending Fc domain (numbering according to Kabat EU index).

In another embodiment the immune activating Fc domain binding molecule of the invention comprises amino acid mutation T366W in the CH3 domain of the first subunit of the half-life extending Fc domain and amino acid mutations T366S, L368A, Y407V in the CH3 domain of the second subunit of the half-life extending Fc domain, and additionally amino acid mutations R409D; K370E in the CH3 domain of the first subunit of the half-life extending Fc domain and amino acid mutations D399K; E357K in the CH3 domain of the second subunit of the half-life extending Fc domain (numberings according to Kabat EU index).

In another embodiment immune activating Fc domain binding molecule of the invention comprises amino acid mutations S354C, T366W in the CH3 domain of the first subunit of the half-life extending Fc domain and amino acid mutations Y349C, T366S, L368A, Y407V in the CH3 domain of the second subunit of the half-life extending Fc domain, or said immune activating Fc domain binding molecule comprises amino acid mutations Y349C, T366W in the CH3 domain of the first subunit of the half-life extending Fc domain and amino acid mutations S354C, T366S, L368A, Y407V in the CH3 domains of the second subunit of the half-life extending Fc domain and additionally amino acid mutations R409D; K370E in the CH3 domain of the first subunit of the Fc domain and amino acid mutations D399K; E357K in the CH3 domain of the second subunit of the Fc domain (all numberings according to Kabat EU index).

In one embodiment the heterodimerization approach described in WO 2013/157953 is used alternatively. In one embodiment a first CH3 domain comprises amino acid mutation T366K and a second CH3 domain comprises amino acid mutation L351D (numberings according to Kabat EU index). In a further embodiment the first CH3 domain comprises further amino acid mutation L351K. In a further embodiment the second CH3 domain comprises further an amino acid mutation selected from Y349E, Y349D and L368E (preferably L368E) (numberings according to Kabat EU index).

In one embodiment the heterodimerization approach described in WO 2012/058768 is used alternatively. In one embodiment a first CH3 domain comprises amino acid mutations L351Y, Y407A and a second CH3 domain comprises amino acid mutations T366A, K409F. In a further embodiment the second CH3 domain comprises a further amino acid mutation at position T411, D399, S400, F405, N390, or K392, e.g. selected from a) T411N, T411R, T411Q, T411K, T411D, T411E or T411W, b) D399R, D399W, D399Y or D399K, c) S400E, S400D, S400R, or S400K, d) F405I, F405M, F405T, F405S, F405V or F405W, e) N390R, N390K or N390D, f) K392V, K392M, K392R, K392L, K392F or K392E (numberings according to Kabat EU index). In a further embodiment a first CH3 domain comprises amino acid mutations L351Y, Y407A and a second CH3 domain comprises amino acid mutations T366V, K409F. In a further embodiment a first CH3 domain comprises amino acid mutation Y407A and a second CH3 domain comprises amino acid mutations T366A, K409F. In a further embodiment the second CH3 domain further comprises amino acid mutations K392E, T411E, D399R and S400R (numberings according to Kabat EU index).

In one embodiment the heterodimerization approach described in WO 2011/143545 is used alternatively, e.g. with the amino acid modification at a position selected from the group consisting of 368 and 409 (numbering according to Kabat EU index).

In one embodiment the heterodimerization approach described in WO 2011/090762, which also uses the knobs-into-holes technology described above, is used alternatively. In one embodiment a first CH3 domain comprises amino acid mutation T366W and a second CH3 domain comprises amino acid mutation Y407A. In one embodiment a first CH3 domain comprises amino acid mutation T366Y and a second CH3 domain comprises amino acid mutation Y407T (numberings according to Kabat EU index).

In one embodiment the half-life extending Fc domain is of IgG₂ subclass and the heterodimerization approach described in WO 2010/129304 is used alternatively.

In an alternative embodiment a modification promoting association of the first and the second subunit of the half-life extending Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable. In one such embodiment a first CH3 domain comprises amino acid substitution of K392 or N392 with a negatively charged amino acid (e.g. glutamic acid (E), or aspartic acid (D), preferably K392D or N392D) and a second CH3 domain comprises amino acid substitution of D399, E356, D356, or E357 with a positively charged amino acid (e.g. lysine (K) or arginine (R), preferably D399K, E356K, D356K, or E357K, and more preferably D399K and E356K). In a further embodiment the first CH3 domain further comprises amino acid substitution of K409 or R409 with a negatively charged amino acid (e.g. glutamic acid (E), or aspartic acid (D), preferably K409D or R409D). In a further embodiment the first CH3 domain further or alternatively comprises amino acid substitution of K439 and/or K370 with a negatively charged amino acid (e.g. glutamic acid (E), or aspartic acid (D)) (all numberings according to Kabat EU index).

In yet a further embodiment the heterodimerization approach described in WO 2007/147901 is used alternatively. In one embodiment a first CH3 domain comprises amino acid mutations K253E, D282K, and K322D and a second CH3 domain comprises amino acid mutations D239K, E240K, and K292D (numberings according to Kabat EU index).

In still another embodiment the heterodimerization approach described in WO 2007/110205 can be used alternatively.

In one embodiment, the first subunit of the Fc domain comprises amino acid substitutions K392D and K409D, and the second subunit of the Fc domain comprises amino acid substitutions D356K and D399K (numbering according to Kabat EU index).

Fc Domain Binding Moiety

The immune activating Fc domain binding molecule of the invention comprises at least on Fc domain binding moiety which specifically binds to the target Fc domain as illustrated in FIG. 1. Accordingly, immune activating Fc domain binding molecules of the invention are capable of specific binding to the target Fc domain of a targeting antibody, i.e. a therapeutic antibody. As herein described the present invention provides a versatile platform to direct specific effector functions to target cells. The targeting antibody recognizes and binds to the target cell. The immune activating Fc domain binding molecule of the invention recognizes and binds to the target Fc domain comprised in the targeting antibody. The target Fc domain confers to the targeting antibodies, i.e. therapeutic antibodies, favorable pharmacokinetic properties, including a long serum half-life which contributes to good accumulation in the target tissue and a favorable tissue-blood distribution ratio. At the same time it may, however, lead to undesirable targeting of therapeutic antibodies to cells expressing Fc receptors rather than to the preferred antigen-bearing cells. Moreover, the co-activation of Fc receptor signaling pathways may lead to cytokine release which, results in excessive activation of cytokine receptors and severe side effects upon systemic administration of therapeutic antibodies. Activation of (Fc receptor-bearing) immune cells other than T cells may even reduce efficacy of therapeutic antibodies due to the potential destruction of immune cells. Accordingly, therapeutic antibodies known in the art may be engineered or mutated to exhibit reduced binding affinity to an Fc receptor and/or reduced effector function, as compared to, e.g., a native IgG₁ Fc domain.

In preferred aspect of the present invention, the targeting antibody is engineered or mutated to exhibit reduced binding affinity to an Fc receptor and/or reduced effector function. As herein above described the target Fc domain may comprise a first set of at least one amino acid substitution. Hence, in preferred embodiments, the targeting antibody has reduced binding affinity to an Fc receptor and/or reduced effector function. At the same time the amino acid substitutions in the first set of at least one amino acid substitutions are used to specifically target the target Fc domain via the Fc domain binding moiety. Fc domain binding moieties with the desirable specificity are herein below described and methods to generate further Fc domain binding moieties with the desired specificity are also herein below described (e.g. immunization of a mammalian immune system with an Fc domain comprising the first set of at least one amino acid substitution, see e.g.

WO2017/072210 incorporated herein by reference). In a preferred embodiment, the Fc domain binding moiety does not specifically bind to the half-life extending Fc domain (to avoid cross-lining of two or more immune activating Fc domain binding molecules of the invention). In such embodiments, it might be desirable to incorporate amino acid substitutions at the same amino acid positions in the target Fc domain and in the half-life extending Fc domain. In one such embodiment, the first set of at least one amino acid substitution as herein before described reduces binding affinity to an Fc receptor and/or effector function, and the second set of at least one amino acid substitution as herein before described comprises one or more amino acid substitutions at the same amino acid positions as in the first set of at least one amino acid substitution, wherein the amino acids in the second set of at least one amino acid substitution are substituted with different amino acids at the same positions compared to the first set of at least one amino acid substitution In a preferred embodiment, the Fc domain binding moiety does not bind to the half-life extending Fc domain. Fc domain binding moieties with such desirable specificity can be generated as herein described, e.g. by immunization of a mammalian immune system with an Fc domain comprising the first set of at least one amino acid substitution and screening for Fc domain binding moieties which do not specifically bind to an Fc domain comprising the second set of at least one amino acid substitution wherein the first and/or second set of at least one amino acid substitution increase binding to an Fc receptor and/or increase effector function.

An exemplary Fc domain binding moiety which specifically binds to a target Fc domain (wherein the first set of at least one amino acid substitutions comprises the P329G substitution) but not to the half-life extending Fc domain (wherein the second set of at least one amino acid substitutions does not comprise the P329G substitution, i.e. is wildtype at the P329 position or comprises an amino acid substitution at position P329 other than glycine) is the anti-P329G (M-1.7.24) huIgG1 binder comprising the CDR sequences of SEQ ID NO: 1, 2, 3, 4, 5 and 6 (numbering according to Kabat EU index) and as further described in WO2017/072210. Another exemplary Fc domain binding moiety which specifically binds to a target Fc domain but not to the half-life extending Fc domain is the anti-AAA binder comprising the CDR sequences of SEQ ID NO: 168, 169, 170, 171, 172, 173 (numbering according to Kabat EU index) and as further described in WO2017/072210. In a preferred embodiment of the present invention, provided are immune activating Fc domain binding molecules comprising an Fc domain binding moiety capable of specific binding to a mutated Fc domain comprising the amino acid substitution P329G. The P329G mutation reduces binding to Fcγ receptors and associated effector function. Accordingly, the mutated Fc domain comprising the P329G substitution binds to Fcγ receptors with reduced or abolished affinity compared to the non-substituted Fc domain. In a preferred embodiment, the Fc domain binding moiety is not capable of binding to an Fc domain comprising an amino acid substitution at position P329 by an amino acid other than glycine (G) (numbering according to Kabat EU index. In one embodiment, the Fc domain binding moiety is not capable of binding to an Fc domain comprising a substitution at position P329 (numbering according to Kabat EU index) by an amino acid other than glycine (G) wherein such amino acid is not able to form a proline sandwich between two conserved tryptophan sidechains within a Fc gamma receptor, in particular within FcgRIIIa. In a preferred embodiment, the Fc domain binding moiety is capable of binding to an Fc domain comprising the amino acid mutation P329G but not capable of binding to an Fc domain comprising an amino acid substitution at position P329 by an amino acid selected from the list consisting of arginine (R), leucine (L), isoleucine (I), and alanine (A).

In particular embodiments, the first set of at least one amino acid substitution comprises an amino acid substitution at position P329 (in an IgG1 Fc). In a preferred embodiment, the first set of at least one amino acid substitution comprises the amino acid substitution P329G in an IgG1 Fc (numbering according to Kabat EU index). In one embodiment the Fc domain binding moiety is capable of specific binding to an IgG1 Fc domain comprising the amino acid substitution P329G (numbering according to Kabat EU index).

In one embodiment, the Fc domain binding moiety capable of specific binding to an IgG1 Fc domain comprising the amino acid substitution P329G (numbering according to Kabat EU index) comprises:
(i) a heavy chain variable region (VH) comprising at least one heavy chain complementary-determinin region selected from the group consisting of SEQ ID NO:1, SEQ ID NO:2, SEQ ID NO:3, SEQ ID NO:11, SEQ ID NO:16 and SEQ ID NO:21; and
(ii) a light chain variable region (VL) comprising at least one light chain complementary-determining region selected from the group consisting of SEQ ID NO:4, SEQ ID NO:5, SEQ ID NO:6 and SEQ ID NO:26.

In one embodiment, the Fc domain binding moiety capable of specific binding to an IgG1 Fc domain comprising the amino acid substitution P329G (numbering according to Kabat EU index) comprises:
(i) a heavy chain variable region (VH) comprising
   (a) the heavy chain complementarity-determining region (CDR H) 1 amino acid sequence RYWMN (SEQ ID NO:1);
   (b) a CDR H2 amino acid sequence selected from the group consisting of EITPDSSTINYTPSLKD (SEQ ID NO:2), EITPDSSTINYTPSLKG (SEQ ID NO:11) and EITPDSSTINYAPSLKG (SEQ ID NO:16); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

In one particular embodiment, the Fc domain binding moiety capable of specific binding to an IgG1 Fc domain comprising the amino acid substitution P329G (numbering according to Kabat EU index) comprises:
(i) a heavy chain variable region (VH) comprising
   (a) the heavy chain complementarity-determining region (CDR H) 1 amino acid sequence RYWMN (SEQ ID NO:1);
   (b) the CDR H2 amino acid sequence EITPDSSTINYTPSLKD (SEQ ID NO:2); and
   (c) the CDR H3 amino acid sequence PYDYGAWFAS (SEQ ID NO:3); and
(ii) a light chain variable region (VL) comprising
(d) the light chain complementary-determining region (CDR L) 1 amino acid sequence RSSTGAVTTSNYAN (SEQ ID NO:4);
(e) the CDR L2 amino acid sequence GTNKRAP (SEQ ID NO:5); and
(f) the CDR L3 amino acid sequence ALWYSNHWV (SEQ ID NO:6). In one particular embodiment, the Fc domain binding moiety capable of specific binding to an IgG1 Fc domain comprising the amino acid substitution P329G (numbering according to Kabat EU index) comprises:
(i) a heavy chain variable region (VH) comprising
   (a) the heavy chain complementarity-determining region (CDR H) 1 amino acid sequence RYWMN (SEQ ID NO:1);
   (b) the CDR H2 amino acid sequence EITPDSSTINYTPSLKG (SEQ ID NO:11); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

In one embodiment, the Fc domain binding moiety capable of specific binding to an IgG1 Fc domain comprising the amino acid substitution P329G (numbering according to Kabat EU index) comprises:
(i) a heavy chain variable region (VH) comprising
   (a) the heavy chain complementarity-determining region (CDR H) 1 amino acid sequence RYWMN (SEQ ID NO:1);
   (b) the CDR H2 amino acid sequence EITPDSSTINYAPSLKG (SEQ ID NO:16); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

In one embodiment, the Fc domain binding moiety capable of specific binding to an IgG1 Fc domain comprising the amino acid substitution P329G (numbering according to Kabat EU index) comprises a heavy chain variable region sequence that is at least about 95%, 96%, 97%, 98%, 99% or 100% identical to an amino acid sequence selected from the group consisting of SEQ ID NO:7, SEQ ID NO:12, SEQ ID NO:17 and SEQ ID NO:19, and a light chain variable region sequence that is at least about 95%, 96%, 97%, 98%, 99% or 100% identical to an amino acid sequence selected from the group consisting of SEQ ID NO:8 and SEQ ID NO:13.

In one embodiment, the Fc domain binding moiety capable of specific binding to an IgG1 Fc domain comprising the amino acid substitution P329G (numbering according to Kabat EU index) comprises
(i) a heavy chain variable region sequence that is at least about 95%, 96%, 97%, 98%, 99% or 100% identical to SEQ ID NO: 7 and a light chain variable region sequence that is at least about 95%, 96%, 97%, 98%, 99% or 100% identical to SEQ ID NO: 8,
(ii) a heavy chain variable region sequence that is at least about 95%, 96%, 97%, 98%, 99% or 100% identical to SEQ ID NO: 12 and a light chain variable region sequence that is at least about 95%, 96%, 97%, 98%, 99% or 100% identical to SEQ ID NO: 13,
(iii) a heavy chain variable region sequence that is at least about 95%, 96%, 97%, 98%, 99% or 100% identical to SEQ ID NO: 17 and a light chain variable region sequence that is at least about 95%, 96%, 97%, 98%, 99% or 100% identical to SEQ ID NO: 13, or
(iv) a heavy chain variable region sequence that is at least about 95%, 96%, 97%, 98%, 99% or 100% identical to SEQ ID NO: 19 and a light chain variable region sequence that is at least about 95%, 96%, 97%, 98%, 99% or 100% identical to SEQ ID NO: 13.

In one embodiment, the Fc domain binding moiety capable of specific binding to an IgG1 Fc domain comprising the amino acid substitution P329G (numbering according to Kabat EU index) comprises the heavy chain variable region sequence of SEQ ID NO: 7 and the light chain variable of SEQ ID NO: 8.

In a preferred embodiment, the Fc domain binding moiety capable of specific binding to an IgG1 Fc domain comprising the amino acid substitution P329G (numbering according to Kabat EU index) comprises the heavy chain variable region sequence of SEQ ID NO: 12 and the light chain variable of SEQ ID NO: 13.

In another preferred embodiment, the Fc domain binding moiety capable of specific binding to an IgG1 Fc domain comprising the amino acid substitution P329G (numbering according to Kabat EU index) comprises the heavy chain variable region sequence of SEQ ID NO: 17 and the light chain variable of SEQ ID NO: 13.

In another preferred embodiment, the Fc domain binding moiety capable of specific binding to an IgG1 Fc domain comprising the amino acid substitution P329G (numbering according to Kabat EU index) comprises the heavy chain variable region sequence of SEQ ID NO: 19 and the light chain variable of SEQ ID NO: 13.

In another embodiment, the Fc domain binding moiety is capable of binding to an Fc domain comprising the amino acid substitutions I253A, H310A and H435A (numbering according to Kabat EU index). In one embodiment, the Fc domain binding moiety is not capable of binding to an Fc domain comprising an amino acid substitution at positions I253, H310 and H435 by an amino acid other than alanine (A) (numbering according to Kabat EU index). In one embodiment, the Fc domain binding moiety is capable of binding to an Fc domain comprising the amino acid substitutions I253A, H310A and H435A but not capable of binding to an Fc domain comprising an amino acid substitution at position I253, H310 and H435 by an amino acid other than alanine (A) (numbering according to Kabat EU index).

In one embodiment, the first set of at least one amino acid substitution comprises an amino acid substitution at positions I253A, H310A and H435A in an IgG1 Fe (numbering according to Kabat EU index). In one embodiment, the first set of at least one amino acid substitution comprises the amino acid substitutions I253A, H310A and H435A in an IgG1 Fc (numbering according to Kabat EU index). In one embodiment the Fc domain binding moiety is capable of specific binding to an IgG1 Fc domain comprising the amino acid substitutions I253A, H310A and H435A (numbering according to Kabat EU index).

In another embodiment, the Fc domain binding moiety capable of specific binding to an IgG1 Fc domain comprising the amino acid mutations I253A, H310A and H435A (numbering according to Kabat EU index) comprises:
(i) a heavy chain variable region (VH) comprising
   (a) the heavy chain complementarity-determining region (CDR H) 1 amino acid sequence SYGMS (SEQ ID NO:168);
   (b) the CDR H2 amino acid sequence SSGGSY (SEQ ID NO:169); and
   (c) the CDR H3 amino acid sequence LGMITTGYAMDY (SEQ ID NO:170); and
(ii) a light chain variable region (VL) comprising
   (d) the light chain complementary-determining region (CDR L) 1 amino acid sequence RSSQTIVHSTGHTYLE (SEQ ID NO:171);
   (e) the CDR L2 amino acid sequence KVSNRFS (SEQ ID NO:172); and
   (f) the CDR L3 amino acid sequence ALWYSNHWV FQGSHVPYT (SEQ ID NO:173).

In one embodiment, the Fc domain binding moiety capable of specific binding to an IgG1 Fc domain comprising the amino acid mutations I253A, H310A and H435A (numbering according to Kabat EU index) comprises a heavy chain variable region sequence that is at least about 95%, 96%, 97%, 98%, 99% or 100% identical to SEQ ID NO: 174 and a light chain variable region sequence that is at least about 95%, 96%, 97%, 98%, 99% or 100% identical to SEQ ID NO: 175.

In one embodiment, the Fc domain binding moiety capable of specific binding to an IgG1 Fc domain comprising the amino acid mutations I253A, H310A and H435A (numbering according to Kabat EU index) comprises the heavy chain variable region sequence of SEQ ID NO: 174 and the light chain variable of SEQ ID NO: 175.

Bispecific Immune Activating Fc Domain Binding Molecules of the Invention

In a further aspect, the invention provides bispecific immune activating Fc domain binding molecules, i.e, the immune activating moiety is an antigen binding moiety (e.g. a Fab molecule).

Accordingly, the invention provides an immune activating Fc domain binding molecule comprising
(a) an Fc domain binding moiety which specifically binds to a target Fc domain comprising a first set of at least one amino acid substitution as herein described,
(b) an immune activating moiety which is a Fab molecule, a scFv molecule or a scFab molecule, and
(c) a half-life extending Fc domain composed of a first and a second subunit capable of stable association,
wherein the Fc domain binding moiety does not specifically bind to the half-life extending Fc domain as herein described.

The components of the immune activating fragment crystallizable (Fc) domain binding molecule can be fused to each other in a variety of configurations. Exemplary configurations are depicted in FIG. 2. In some embodiments, the immune activating moiety is a Fab molecule fused at the C-terminus of the Fab heavy chain to the N-terminus of the first or the second subunit of the half-life extending Fc domain. In one such embodiment, the Fc domain binding moiety is a Fab molecule fused at the C-terminus of the Fab heavy chain to the N-terminus of the Fab heavy chain of the immune activating moiety which is a second Fab molecule. In a specific such embodiment, the immune activating Fc domain binding molecule essentially consists of the first and the second Fab molecule, the half-life extending Fc domain composed of a first and a second subunit, and optionally one or more peptide linkers, wherein the first Fab molecule is fused at the C-terminus of the Fab heavy chain to the N-terminus of the Fab heavy chain of the second Fab molecule, and the second Fab molecule is fused at the C-terminus of the Fab heavy chain to the N-terminus of the first or the second subunit of the half-life extending Fc domain. Such a configuration is schematically depicted in FIG. 2G and FIG. 2K. Optionally, the Fab light chain of the first Fab molecule and the Fab light chain of the second Fab molecule may additionally be fused to each other.

In another specific such embodiment, the immune activating Fc domain binding molecule essentially consists of the Fc domain binding moiety which is a Fab molecule and the immune activating moiety which is a second Fab molecule, the half-life extending Fc domain composed of a first and a second subunit, and optionally one or more peptide linkers, wherein the first and the second Fab molecule are each fused at the C-terminus of the Fab heavy chain to the N-terminus of one of the subunits of the Fc domain. Such a configuration is schematically depicted in FIG. 2A and FIG. 2D. The first and the second Fab molecule may be fused to the half-life extending Fc domain directly or through a peptide linker. In a particular embodiment the first and the second Fab molecule are each fused to the Fc domain through an immunoglobulin hinge region. In a specific embodiment, the immunoglobulin hinge region is a human IgG₁ hinge region, particularly where the Fc domain is an IgG₁ Fc domain.

In other embodiments, the Fc domain binding moiety is a Fab molecule fused at the C-terminus of the Fab heavy chain to the N-terminus of the first or second subunit of the half-life extending Fc domain. In one such embodiment, the immune activating moiety is a second Fab molecule fused at the C-terminus of the Fab heavy chain to the N-terminus of the Fab heavy chain of the first Fab molecule. In a specific such embodiment, the immune activating Fc domain binding molecule essentially consists of the first and the second Fab molecule, the Fc domain composed of a first and a second subunit, and optionally one or more peptide linkers, wherein the second Fab molecule is fused at the C-terminus of the Fab heavy chain to the N-terminus of the Fab heavy chain of the first Fab molecule, and the first Fab molecule is fused at the C-terminus of the Fab heavy chain to the N-terminus of the first or the second subunit of the Fc domain. Such a configuration is schematically depicted in FIG. 2H and FIG. 2L. Optionally, the Fab light chain of the first Fab molecule and the Fab light chain of the second Fab molecule may additionally be fused to each other.

The Fab molecules may be fused to the half-life extending Fc domain or to each other directly or through a peptide linker, comprising one or more amino acids, typically about 2-20 amino acids. Peptide linkers are known in the art and are described herein. Suitable, non-immunogenic peptide linkers include, for example, (G₄S)ₙ, (SG₄)ₙ, (G₄S)ₙ or G₄(SG₄)ₙ peptide linkers. “n” is generally an integer from 1 to 10, typically from 2 to 4. In one embodiment said peptide linker has a length of at least 5 amino acids, in one embodiment a length of 5 to 100, in a further embodiment of 10 to 50 amino acids. In one embodiment said peptide linker is (GxS)ₙ or (GxS)ₙGₘ with G=glycine, S=serine, and (x=3, n=3, 4, 5 or 6, and m=0, 1, 2 or 3) or (x=4, n=2, 3, 4 or 5 and m=0, 1, 2 or 3), in one embodiment x=4 and n=2 or 3, in a further embodiment x=4 and n=2. In one embodiment said peptide linker is (G₄S)₂. A particularly suitable peptide linker for fusing the Fab light chains of the first and the second Fab molecule to each other is (G₄S)₂. An exemplary peptide linker suitable for connecting the Fab heavy chains of the first and the second Fab fragments comprises the sequence (D)-(G₄S)₂). Another exemplary peptide linker suitable for connecting the Fab heavy chains of the first and the second Fab fragments comprises the sequence (G₄SG₅). Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In some cases, it will be advantageous to have an immune activating Fc domain binding molecule comprising two or more Fc domain binding moieties as herein described (see examples shown in FIG. 2B, FIG. 2C, FIG. 2E, FIG. 2F, FIG. 2I, FIG. 2J, FIG. 2M or FIG. 2N), for example to optimize targeting to the target Fc domain or to allow crosslinking of target molecules.

Accordingly, in particular embodiments, the immune activating Fc domain binding molecule of the invention further comprises a third Fab molecule which specifically binds to a target Fc domain comprising a first set of at least one amino acid substitution as herein described. In one embodiment, the third Fab molecule is a conventional Fab molecule. In one embodiment, the third Fab molecule is identical to the first Fab molecule (i.e. the first and the third Fab molecule comprise the same heavy and light chain amino acid sequences and have the same arrangement of domains (i.e. conventional or crossover)). In a particular embodiment, the second Fab molecule specifically binds to an immune activating antigen, particularly CD3, and the first and third Fab molecule specifically bind to a target Fc domain comprising a first set of at least one amino acid substitution as herein described.

In alternative embodiments, the immune activating Fc domain binding molecule of the invention further comprises a third Fab molecule which specifically binds to an immune activating antigen, particularly CD3. In one such embodiment, the third Fab molecule is a crossover Fab molecule (a Fab molecule wherein the variable domains VH and VL of the Fab heavy and light chains are exchanged/replaced by each other). In one such embodiment, the third Fab molecule is identical to the second Fab molecule (i.e. the second and the third Fab molecule comprise the same heavy and light chain amino acid sequences and have the same arrangement of domains (i.e. conventional or crossover)). In one such embodiment, the first Fab molecule specifically binds to an immune activating antigen, particularly CD3, and the second and third Fab molecule specifically bind to target Fc domain comprising a first set of at least one amino acid substitution as herein described. In one embodiment, the third Fab molecule is fused at the C-terminus of the Fab heavy chain to the N-terminus of the first or second subunit of the Fc domain.

In a particular embodiment, the second and the third Fab molecule are each fused at the C-terminus of the Fab heavy chain to the N-terminus of one of the subunits of the Fc domain, and the first Fab molecule is fused at the C-terminus of the Fab heavy chain to the N-terminus of the Fab heavy chain of the second Fab molecule. In a specific such embodiment, the immune activating Fc domain binding molecule essentially consists of the first, the second and the third Fab molecule, the half-life extending Fc domain composed of a first and a second subunit, and optionally one or more peptide linkers, wherein the first Fab molecule is fused at the C-terminus of the Fab heavy chain to the N-terminus of the Fab heavy chain of the second Fab molecule, and the second Fab molecule is fused at the C-terminus of the Fab heavy chain to the N-terminus of the first subunit of the half-life extending Fc domain, and wherein the third Fab molecule is fused at the C-terminus of the Fab heavy chain to the N-terminus of the second subunit of the half-life extending Fc domain. Such a configuration is schematically depicted in FIG. 2B and FIG. 2E (particular embodiments, wherein the third Fab molecule is a conventional Fab molecule and preferably identical to the first Fab molecule), and FIG. 2I and FIG. 2M (alternative embodiments, wherein the third Fab molecule is a crossover Fab molecule and preferably identical to the second Fab molecule). The second and the third Fab molecule may be fused to the half-life extending Fc domain directly or through a peptide linker. In a particular embodiment the second and the third Fab molecule are each fused to the half-life extending Fc domain through an immunoglobulin hinge region. In a specific embodiment, the immunoglobulin hinge region is a human IgG₁ hinge region, particularly where the half-life extending Fc domain is an IgG₁ Fc domain. Optionally, the Fab light chain of the first Fab molecule and the Fab light chain of the second Fab molecule may additionally be fused to each other.

In another embodiment, the first and the third Fab molecule are each fused at the C-terminus of the Fab heavy chain to the N-terminus of one of the subunits of the half-life extending Fc domain, and the second Fab molecule is fused at the C-terminus of the Fab heavy chain to the N-terminus of the Fab heavy chain of the first Fab molecule. In a specific such embodiment, the immune activating Fc domain binding molecule essentially consists of the first, the second and the third Fab molecule, the half-life extending Fc domain composed of a first and a second subunit, and optionally one or more peptide linkers, wherein the second Fab molecule is fused at the C-terminus of the Fab heavy chain to the N-terminus of the Fab heavy chain of the first Fab molecule, and the first Fab molecule is fused at the C-terminus of the Fab heavy chain to the N-terminus of the first subunit of the Fc domain, and wherein the third Fab molecule is fused at the C-terminus of the Fab heavy chain to the N-terminus of the second subunit of the Fc domain. Such a configuration is schematically depicted in FIG. 2C and FIG. 2F (particular embodiments, wherein the third Fab molecule is a conventional Fab molecule and preferably identical to the first Fab molecule) and in FIG. 2J and FIG. 2N (alternative embodiments, wherein the third Fab molecule is a crossover Fab molecule and preferably identical to the second Fab molecule). The first and the third Fab molecule may be fused to the half-life extending Fc domain directly or through a peptide linker.

In a particular embodiment the first and the third Fab molecule are each fused to the half-life extending Fc domain through an immunoglobulin hinge region. In a specific embodiment, the immunoglobulin hinge region is a human IgG1 hinge region, particularly where the Fc domain is an IgG₁ Fc domain. Optionally, the Fab light chain of the first Fab molecule and the Fab light chain of the second Fab molecule may additionally be fused to each other.

In configurations of the immune activating Fc domain binding molecule wherein a Fab molecule is fused at the C-terminus of the Fab heavy chain to the N-terminus of each of the subunits of the half-life extending Fc domain through an immunoglobulin hinge regions, the two Fab molecules, the hinge regions and the half-life extending Fc domain essentially form an immunoglobulin molecule. In a particular embodiment the immunoglobulin molecule is an IgG class immunoglobulin. In an even more particular embodiment the immunoglobulin is an IgG₁ subclass immunoglobulin. In another embodiment the immunoglobulin is an IgG₄ subclass immunoglobulin. In a further particular embodiment the immunoglobulin is a human immunoglobulin. In other embodiments the immunoglobulin is a chimeric immunoglobulin or a humanized immunoglobulin.

In some of the immune activating Fc domain binding molecule of the invention, the Fab light chain of the first Fab molecule and the Fab light chain of the second Fab molecule are fused to each other, optionally via a peptide linker. Depending on the configuration of the first and the second Fab molecule, the Fab light chain of the first Fab molecule may be fused at its C-terminus to the N-terminus of the Fab light chain of the second Fab molecule, or the Fab light chain of the second Fab molecule may be fused at its C-terminus to the N-terminus of the Fab light chain of the first Fab molecule. Fusion of the Fab light chains of the first and the second Fab molecule further reduces mispairing of unmatched Fab heavy and light chains, and also reduces the number of plasmids needed for expression of some of the immune activating Fc domain binding molecule of the invention.

In certain embodiments the immune activating Fc domain binding molecule according to the invention comprises a polypeptide wherein the Fab light chain variable region of the second Fab molecule shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region), which in turn shares a carboxy-terminal peptide bond with a Fc domain subunit (VL₍₂₎-CH1₍₂₎-CH2-CH3(-CH4)), and a polypeptide wherein the Fab heavy chain of the first Fab molecule shares a carboxy-terminal peptide bond with an Fc domain subunit (VH₍₁₎-CH1₍₁₎-CH2-CH3(-CH4)). In some embodiments the immune activating Fc domain binding molecule further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In certain embodiments the polypeptides are covalently linked, e.g., by a disulfide bond.

In some embodiments, the immune activating Fc domain binding molecule comprises a polypeptide wherein the Fab light chain variable region of the second Fab molecule shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region), which in turn shares a carboxy-terminal peptide bond with the Fab heavy chain of the first Fab molecule, which in turn shares a carboxy-terminal peptide bond with an Fc domain subunit (VL₍₂₎-CH1₍₂₎-VH₍₁₎-CH1₍₁₎-CH2-CH3(-CH4)). In other embodiments, the immune activating Fc domain binding molecule comprises a polypeptide wherein the Fab heavy chain of the first Fab molecule shares a carboxy-terminal peptide bond with the Fab light chain variable region of the second Fab molecule which in turn shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region), which in turn shares a carboxy-terminal peptide bond with an Fc domain subunit (VH₍₁₎-CH1₍₁₎-VL₍₂₎-CH1₍₂₎-CH2-CH3(-CH4)).

In some of these embodiments the immune activating Fc domain binding molecule further comprises a crossover Fab light chain polypeptide of the second Fab molecule, wherein the Fab heavy chain variable region of the second Fab molecule shares a carboxy-terminal peptide bond with the Fab light chain constant region of the second Fab molecule (VH₍₂₎-CL₍₂₎), and the Fab light chain polypeptide of the first Fab molecule (VL₍₁₎-CL₍₁₎). In others of these embodiments the immune activating Fc domain binding molecule further comprises a polypeptide wherein the Fab light chain variable region of the second Fab molecule shares a carboxy-terminal peptide bond with the Fab heavy chain constant region of the second Fab molecule which in turn shares a carboxy-terminal peptide bond with the Fab light chain polypeptide of the first Fab molecule (VL₍₂₎-CH1₍₂₎-VL₍₁₎-CL₍₁₎), or a polypeptide wherein the Fab light chain polypeptide of the first Fab molecule shares a carboxy-terminal peptide bond with the Fab heavy chain variable region of the second Fab molecule which in turn shares a carboxy-terminal peptide bond with the Fab light chain constant region of the second Fab molecule (VL₍₁₎-CL₍₁₎-VH₍₂₎-CL₍₂₎), as appropriate.

The immune activating Fc domain binding molecule according to these embodiments may further comprise (i) an Fc domain subunit polypeptide (CH2-CH3(-CH4)), or (ii) a polypeptide wherein the Fab heavy chain of a third Fab molecule shares a carboxy-terminal peptide bond with an Fc domain subunit (VH₍₃₎-CH1₍₃₎-CH2-CH3(-CH4)) and the Fab light chain polypeptide of a third Fab molecule (VL₍₃₎-CL₍₃₎). In certain embodiments the polypeptides are covalently linked, e.g., by a disulfide bond.

In some aspect, the immune activating Fc domain binding molecule does not comprise an Fc domain for example if a short half-life of the immune acrivating Fc domain binding molecule is preferred. Accordingly, the present invention provides immune activating Fc domain binding molecules devoid of an Fc domain (for illustrative formats see FIG. 2O-FIG. 2Z). In some embodiments, the first Fab molecule is fused at the C-terminus of the Fab heavy chain to the N-terminus of the Fab heavy chain of the second Fab molecule. In certain such embodiments, the immune activating Fc domain binding molecule does not comprise an Fc domain. In certain embodiments, the immune activating Fc domain binding molecule essentially consists of the first and the second Fab molecule, and optionally one or more peptide linkers, wherein the first Fab molecule is fused at the C-terminus of the Fab heavy chain to the N-terminus of the Fab heavy chain of the second Fab molecule. Such a configuration is schematically depicted in FIG. 2O and FIG. 2S. In other embodiments, the second Fab molecule is fused at the C-terminus of the Fab heavy chain to the N-terminus of the Fab heavy chain of the first Fab molecule. In certain such embodiments, the immune activating Fc domain binding molecule does not comprise an Fc domain. In certain embodiments, the immune activating Fc domain binding molecule essentially consists of the first and the second Fab molecule, and optionally one or more peptide linkers, wherein the second Fab molecule is fused at the C-terminus of the Fab heavy chain to the N-terminus of the Fab heavy chain of the first Fab molecule. Such a configuration is schematically depicted in FIG. 2P and FIG. 2T. In some embodiments, the first Fab molecule is fused at the C-terminus of the Fab heavy chain to the N-terminus of the Fab heavy chain of the second Fab molecule, and immune activating Fc domain binding molecule further comprises a third Fab molecule, wherein said third Fab molecule is fused at the C-terminus of the Fab heavy chain to the N-terminus of the Fab heavy chain of the first Fab molecule. In particular such embodiments, said third Fab molecule is a conventional Fab molecule. In other such embodiments, said third Fab molecule is a crossover Fab molecule as described herein, i.e. a Fab molecule wherein the variable domains VH and VL of the Fab heavy and light chains are exchanged/replaced by each other. In certain such embodiments, the immune activating Fc domain binding molecule essentially consists of the first, the second and the third Fab molecule, and optionally one or more peptide linkers, wherein the first Fab molecule is fused at the C-terminus of the Fab heavy chain to the N-terminus of the Fab heavy chain of the second Fab molecule, and the third Fab molecule is fused at the C-terminus of the Fab heavy chain to the N-terminus of the Fab heavy chain of the first Fab molecule. Such a configuration is schematically depicted in FIG. 2Q and FIG. 2U (particular embodiments, wherein the third Fab molecule is a conventional Fab molecule and preferably identical to the first Fab molecule). In some embodiments, the first Fab molecule is fused at the C-terminus of the Fab heavy chain to the N-terminus of the Fab heavy chain of the second Fab molecule, and the immune activating Fc domain binding molecule further comprises a third Fab molecule, wherein said third Fab molecule is fused at the N-terminus of the Fab heavy chain to the C-terminus of the Fab heavy chain of the second Fab molecule. In particular such embodiments, said third Fab molecule is a crossover Fab molecule as described herein, i.e. a Fab molecule wherein the variable domains VH and VL of the Fab heavy and light chains are exchanged/replaced by each other. In other such embodiments, said third Fab molecule is a conventional Fab molecule. In certain such embodiments, the immune activating Fc domain binding molecule essentially consists of the first, the second and the third Fab molecule, and optionally one or more peptide linkers, wherein the first Fab molecule is fused at the C-terminus of the Fab heavy chain to the N-terminus of the Fab heavy chain of the second Fab molecule, and the third Fab molecule is fused at the N-terminus of the Fab heavy chain to the C-terminus of the Fab heavy chain of the second Fab molecule. Such a configuration is schematically depicted in FIG. 2W and FIG. 2Y (particular embodiments, wherein the third Fab molecule is a crossover Fab molecule and preferably identical to the second Fab molecule). In some embodiments, the second Fab molecule is fused at the C-terminus of the Fab heavy chain to the N-terminus of the Fab heavy chain of the first Fab molecule, and the immune activating Fc domain binding molecule further comprises a third Fab molecule, wherein said third Fab molecule is fused at the N-terminus of the Fab heavy chain to the C-terminus of the Fab heavy chain of the first Fab molecule. In particular such embodiments, said third Fab molecule is a conventional Fab molecule. In other such embodiments, said third Fab molecule is a crossover Fab molecule as described herein, i.e. a Fab molecule wherein the variable domains VH and VL of the Fab heavy and light chains are exchanged/replaced by each other. In certain such embodiments, the immune activating Fc domain binding molecule essentially consists of the first, the second and the third Fab molecule, and optionally one or more peptide linkers, wherein the second Fab molecule is fused at the C-terminus of the Fab heavy chain to the N-terminus of the Fab heavy chain of the first Fab molecule, and the third Fab molecule is fused at the N-terminus of the Fab heavy chain to the C-terminus of the Fab heavy chain of the first Fab molecule. Such a configuration is schematically depicted in FIG. 2R and FIG. 2V (particular embodiments, wherein the third Fab molecule is a conventional Fab molecule and preferably identical to the first Fab molecule). In some embodiments, the second Fab molecule is fused at the C-terminus of the Fab heavy chain to the N-terminus of the Fab heavy chain of the first Fab molecule, and the immune activating Fc domain binding molecule further comprises a third Fab molecule, wherein said third Fab molecule is fused at the C-terminus of the Fab heavy chain to the N-terminus of the Fab heavy chain of the second Fab molecule. In particular such embodiments, said third Fab molecule is a crossover Fab molecule as described herein, i.e. a Fab molecule wherein the variable domains VH and VL of the Fab heavy and light chains are exchanged/replaced by each other. In other such embodiments, said third Fab molecule is a conventional Fab molecule. In certain such embodiments, the immune activating Fc domain binding molecule essentially consists of the first, the second and the third Fab molecule, and optionally one or more peptide linkers, wherein the second Fab molecule is fused at the C-terminus of the Fab heavy chain to the N-terminus of the Fab heavy chain of the first Fab molecule, and the third Fab molecule is fused at the C-terminus of the Fab heavy chain to the N-terminus of the Fab heavy chain of the second Fab molecule. Such a configuration is schematically depicted in FIG. 2X and FIG. 2Z (particular embodiments, wherein the third Fab molecule is a crossover Fab molecule and preferably identical to the first Fab molecule).

In certain embodiments the immune activating Fc domain binding molecule according to the invention comprises a polypeptide wherein the Fab heavy chain of the first Fab molecule shares a carboxy-terminal peptide bond with the Fab light chain variable region of the second Fab molecule, which in turn shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region) (VH₍₁₎-CH1₍₁₎-VL₍₂₎-CH1₍₂₎). In some embodiments the immune activating Fc domain binding molecule further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎).

In certain embodiments the immune activating Fc domain binding molecule according to the invention comprises a polypeptide wherein the Fab light chain variable region of the second Fab molecule shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region), which in turn shares a carboxy-terminal peptide bond with the Fab heavy chain of the first Fab molecule (VL₍₂₎-CH1₍₂₎-VH₍₁₎-CH1₍₁₎). In some embodiments the immune activating Fc domain binding molecule further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎).

In certain embodiments immune activating Fc domain binding molecule according to the invention comprises a polypeptide wherein the Fab heavy chain of a third Fab molecule shares a carboxy-terminal peptide bond with the Fab heavy chain of the first Fab molecule, which in turn shares a carboxy-terminal peptide bond with the Fab light chain variable region of the second Fab molecule, which in turn shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region) (VH₍₃₎-CH1₍₃₎-VH₍₁₎-CH1₍₁₎-VL₍₂₎-CH1₍₂₎). In some embodiments the immune activating Fc domain binding molecule further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some embodiments the immune activating Fc domain binding molecule further comprises the Fab light chain polypeptide of a third Fab molecule (VL₍₃₎-CL₍₃₎).

In certain embodiments the immune activating Fc domain binding molecule according to the invention comprises a polypeptide wherein the Fab light chain variable region of the second Fab molecule shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region), which in turn shares a carboxy-terminal peptide bond with the Fab heavy chain of the first Fab molecule, which in turn shares a carboxy-terminal peptide bond with the Fab heavy chain of a third Fab molecule (VL₍₂₎-CH1₍₂₎-VH₍₁₎-CH1₍₁₎-VH₍₃₎-CH1₍₃₎). In some embodiments the immune activating Fc domain binding molecule further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some embodiments the immune activating Fc domain binding molecule further comprises the Fab light chain polypeptide of a third Fab molecule (VL₍₃₎-CL₍₃₎).

In certain embodiments the immune activating Fc domain binding molecule according to the invention comprises a polypeptide wherein the Fab heavy chain of the first Fab molecule shares a carboxy-terminal peptide bond with the Fab light chain variable region of the second Fab molecule, which in turn shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region), which in turn shares a carboxy-terminal peptide bond with the Fab light chain variable region of a third Fab molecule, which in turn shares a carboxy-terminal peptide bond with the Fab heavy chain constant region of a third Fab molecule (i.e. the third Fab molecule comprises a crossover Fab heavy chain, wherein the heavy chain variable region is replaced by a light chain variable region) (VH₍₁₎-CH1₍₁₎-VL₍₂₎-CH1₍₂₎-VL₍₃₎-CH1₍₃₎). In some embodiments the immune activating Fc domain binding molecule further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some embodiments the immune activating Fc domain binding molecule further comprises a polypeptide wherein the Fab heavy chain variable region of a third Fab molecule shares a carboxy-terminal peptide bond with the Fab light chain constant region of a third Fab molecule (VH₍₃₎-CL₍₃₎).

In certain embodiments the immune activating Fc domain binding molecule according to the invention comprises a polypeptide wherein the Fab light chain variable region of a third Fab molecule shares a carboxy-terminal peptide bond with the Fab heavy chain constant region of a third Fab molecule (i.e. the third Fab molecule comprises a crossover Fab heavy chain, wherein the heavy chain variable region is replaced by a light chain variable region), which in turn shares a carboxy-terminal peptide bond with the Fab light chain variable region of the second Fab molecule, which in turn shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region), which in turn shares a carboxy-terminal peptide bond with the Fab heavy chain of the first Fab molecule (VL₍₃₎-CH1₍₃₎-VL₍₂₎-CH1₍₂₎-VH₍₁₎-CH1₍₁₎). In some embodiments the immune activating Fc domain binding molecule further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some embodiments the immune activating Fc domain binding molecule further comprises a polypeptide wherein the Fab heavy chain variable region of a third Fab molecule shares a carboxy-terminal peptide bond with the Fab light chain constant region of a third Fab molecule (VH₍₃₎-CL₍₃₎).

According to any of the above embodiments, components of the immune activating Fc domain binding molecule (e.g. Fab molecules, Fc domain) may be fused directly or through various linkers, particularly peptide linkers comprising one or more amino acids, typically about 2-20 amino acids, that are described herein or are known in the art. Suitable, non-immunogenic peptide linkers include, for example, (G₄S)ₙ, (SG₄)ₙ, (G₄S)ₙ or G₄(SG₄)ₙ peptide linkers, wherein n is generally an integer from 1 to 10, typically from 2 to 4.

Charge Modifications

In some aspects, the immune activating Fc domain binding molecule of the invention is bispecific, i.e. it comprises at least two antigen binding moieties capable of specific binding to two distinct antigenic determinants. In some aspects, the antigen binding moieties are Fab molecules (i.e. antigen binding domains composed of a heavy and a light chain, each comprising a variable and a constant domain). In one embodiment said Fab molecules are human. In another embodiment said Fab molecules are humanized. In yet another embodiment said Fab molecules comprise human heavy and light chain constant domains.

At least one of the antigen binding moieties is a crossover Fab molecule. Such modification reduces mispairing of heavy and light chains from different Fab molecules, thereby improving the yield and purity of the immune activating Fc domain binding molecule of the invention in recombinant production. In a particular crossover Fab molecule useful for the immune activating Fc domain binding molecule of the invention, the variable domains of the Fab light chain and the Fab heavy chain (VL and VH, respectively) are exchanged. Even with this domain exchange, however, the preparation of the immune activating Fc domain binding molecule may comprise certain side products due to a so-called Bence Jones-type interaction between mispaired heavy and light chains (see Schaefer et al, PNAS, 108 (2011) 11187-11191). To further reduce mispairing of heavy and light chains from different Fab molecules and thus increase the purity and yield of the desired immune activating Fc domain binding molecule, according to the present invention charged amino acids with opposite charges are introduced at specific amino acid positions in the CH1 and CL domains of either the Fab molecule(s) specifically binding to a target cell antigen, or the Fab molecule specifically binding to an immune activating antigen. Charge modifications are made either in the conventional Fab molecule(s) comprised in the immune activating Fc domain binding molecule (such as shown e.g. in FIG. 2A-FIG. 2C, FIG. 2G-FIG. 2J), or in the crossover Fab molecule(s) comprised in the immune activating Fc domain binding molecule (such as shown e.g. in FIG. 2D-FIG. 2F, FIG. 2K-FIG. 2N) (but not in both). In particular embodiments, the charge modifications are made in the conventional Fab molecule(s) comprised in the immune activating Fc domain binding molecule (which in particular embodiments specifically bind(s) to the target cell antigen).

CD3 Binding Immune Activating Fc Domain Binding Molecules

In a particular embodiment according to the invention, the immune activating Fc domain binding molecule is capable of simultaneous binding to an Fc domain binding moiety which specifically binds to a target Fc domain comprising a first set of at least one amino acid substitution as herein above described, and an activating T cell antigen, particularly CD3. The immune activating Fc domain binding molecule of the invention is combined with a targeting antibody comprising an Fc domain comprising the first set of at least one amino acid substitution and at least one antigen binding moiety capable of specific binding to an antigen on a target cell. In such embodiments, the immune activating Fc domain binding molecule is capable of crosslinking a T cell and a target cell by simultaneous binding to the target Fc domain and an activating T cell antigen while the targeting antibody binds to the target cell. In an even more particular embodiment, such simultaneous binding results in lysis of the target cell, particularly a tumor cell. In one embodiment, such simultaneous binding results in activation of the T cell. In other embodiments, such simultaneous binding results in a cellular response of a T lymphocyte, particularly a cytotoxic T lymphocyte, selected from the group of: proliferation, differentiation, cytokine secretion, cytotoxic effector molecule release, cytotoxic activity, and expression of activation markers. In one embodiment, binding of the immune activating Fc domain binding molecule to the activating T cell antigen, particularly CD3, without simultaneous crosslinking to the target cell does not result in T cell activation.

In one embodiment, the immune activating Fc domain binding molecule in combination with the targeting antibody is capable of re-directing cytotoxic activity of a T cell to a target cell. In a particular embodiment, said re-direction is independent of MHC-mediated peptide antigen presentation by the target cell and and/or specificity of the T cell.

Particularly, a T cell according to any of the embodiments of the invention is a cytotoxic T cell. In some embodiments the T cell is a CD4⁺ or a CD8⁺ T cell, particularly a CD8⁺ T cell.

Accordingly, in one aspect of the present invention, the immune activating moiety is an antigen binding moiety capable of specific binding to an activating T cell antigen, in particular CD3. In one embodiment, the immune activating Fc domain binding molecule of the invention comprises at least one Fab molecule which specifically binds to an activating T cell antigen (also referred to herein as an “activating T cell antigen binding Fab molecule”). In a particular embodiment, the immune activating Fc domain binding molecule comprises not more than one Fab molecule (or other Fab molecule) capable of specific binding to an activating T cell antigen. In one embodiment the immune activating Fc domain binding molecule provides monovalent binding to the activating T cell antigen.

In particular embodiments, the Fab molecule which specifically binds an activating T cell antigen is a crossover Fab molecule as described herein, i.e. a Fab molecule wherein the variable domains VH and VL of the Fab heavy and light chains are exchanged/replaced by each other. In such embodiments, the Fab molecule(s) which specifically binds a target Fc domain comprising a first set of at least one amino acid substitution is a conventional Fab molecule. In embodiments where there is more than one Fab molecule which specifically binds to a target Fc domain comprised in the immune activating Fc domain binding molecule, the Fab molecule which specifically binds to an activating T cell antigen preferably is a crossover Fab molecule and the Fab molecules which specifically bind to a target Fc domain are conventional Fab molecules.

In alternative embodiments, the Fab molecule which specifically binds an activating T cell antigen is a conventional Fab molecule. In such embodiments, the Fab molecule(s) which specifically binds a target Fc domain is a crossover Fab molecule as described herein, i.e. a Fab molecule wherein the variable domains VH and VL of the Fab heavy and light chains are exchanged /replaced by each other.

In a particular embodiment the activating T cell antigen is CD3, particularly human CD3. In a particular embodiment the activating T cell antigen binding Fab molecule is cross-reactive for (i.e. specifically binds to) human and cynomolgus CD3. In some embodiments, the activating T cell antigen is the epsilon subunit of CD3 (CD3 epsilon).

In some embodiments, the activating T cell antigen binding Fab molecule specifically binds to CD3, particularly CD3 epsilon, and comprises at least one heavy chain complementarity determining region (CDR) selected from the group consisting of SEQ ID NO: 35, SEQ ID NO: 37 and SEQ ID NO: 43 and at least one light chain CDR selected from the group of SEQ ID NO: 53, SEQ ID NO: 54, SEQ ID NO: 55.

In one embodiment the CD3 binding Fab molecule comprises a heavy chain variable region comprising the heavy chain CDR1 of SEQ ID NO: 35, the heavy chain CDR2 of SEQ ID NO: 37, the heavy chain CDR3 of SEQ ID NO: 43, and a light chain variable region comprising the light chain CDR1 of SEQ ID NO: 53, the light chain CDR2 of SEQ ID NO: 54, and the light chain CDR3 of SEQ ID NO: 55.

In one embodiment the CD3 binding Fab molecule comprises a heavy chain variable region sequence that is at least about 95%, 96%, 97%, 98%, 99% or 100% identical to SEQ ID NO: 49 and a light chain variable region sequence that is at least about 95%, 96%, 97%, 98%, 99% or 100% identical to SEQ ID NO: 56.

In one embodiment the CD3 binding Fab molecule comprises a heavy chain variable region comprising the amino acid sequence of SEQ ID NO: 49 and a light chain variable region comprising the amino acid sequence of SEQ ID NO: 56.

In some embodiments, the activating T cell antigen binding Fab molecule specifically binds to CD3, particularly CD3 epsilon, and comprises at least one heavy chain complementarity determining region (CDR) selected from the group consisting of SEQ ID NO: 34, SEQ ID NO: 37 and SEQ ID NO: 41 and at least one light chain CDR selected from the group of SEQ ID NO: 53, SEQ ID NO: 54, SEQ ID NO: 55.

In one embodiment the CD3 binding Fab molecule comprises a heavy chain variable region comprising the heavy chain CDR1 of SEQ ID NO: 34, the heavy chain CDR2 of SEQ ID NO: 37, the heavy chain CDR3 of SEQ ID NO: 41, and a light chain variable region comprising the light chain CDR1 of SEQ ID NO: 53, the light chain CDR2 of SEQ ID NO: 54, and the light chain CDR3 of SEQ ID NO: 55.

In one embodiment the CD3 binding Fab molecule comprises a heavy chain variable region sequence that is at least about 95%, 96%, 97%, 98%, 99% or 100% identical to SEQ ID NO: 47 and a light chain variable region sequence that is at least about 95%, 96%, 97%, 98%, 99% or 100% identical to SEQ ID NO: 56.

In one embodiment the CD3 binding Fab molecule comprises a heavy chain variable region comprising the amino acid sequence of SEQ ID NO: 47 and a light chain variable region comprising the amino acid sequence of SEQ ID NO: 56.

In some embodiments, the activating T cell antigen binding Fab molecule specifically binds to CD3, particularly CD3 epsilon, and comprises at least one heavy chain complementarity determining region (CDR) selected from the group consisting of SEQ ID NO: 35, SEQ ID NO: 37 and SEQ ID NO: 176 and at least one light chain CDR selected from the group of SEQ ID NO: 53, SEQ ID NO: 54, SEQ ID NO: 55.

In one embodiment the CD3 binding Fab molecule comprises a heavy chain variable region comprising the heavy chain CDR1 of SEQ ID NO: 35, the heavy chain CDR2 of SEQ ID NO: 37, the heavy chain CDR3 of SEQ ID NO: 176, and a light chain variable region comprising the light chain CDR1 of SEQ ID NO: 53, the light chain CDR2 of SEQ ID NO: 54, and the light chain CDR3 of SEQ ID NO: 55.

In one embodiment the CD3 binding Fab molecule comprises a heavy chain variable region sequence that is at least about 95%, 96%, 97%, 98%, 99% or 100% identical to SEQ ID NO: 177 and a light chain variable region sequence that is at least about 95%, 96%, 97%, 98%, 99% or 100% identical to SEQ ID NO: 56.

In one embodiment the CD3 binding Fab molecule comprises a heavy chain variable region comprising the amino acid sequence of SEQ ID NO: 177 and a light chain variable region comprising the amino acid sequence of SEQ ID NO: 56.

In one embodiment, provided is an immune activating fragment crystallizable (Fc) domain binding molecule comprising:
(a) a first light chain comprising an amino acid sequence of SEQ ID NO:86.
(b) a second light chain comprising the amino acid sequence of SEQ ID NO:68
(c) a first heavy chain comprising the amino acid sequence of SEQ ID NO:87; and
(d) a second heavy chain comprising the amino acid sequence of SEQ ID NO:88.

In one embodiment, provided is an immune activating fragment crystallizable (Fc) domain binding molecule comprising:
(a) a first light chain comprising an amino acid sequence of SEQ ID NO:89.
(b) a second light chain comprising the amino acid sequence of SEQ ID NO:68
(c) a first heavy chain comprising the amino acid sequence of SEQ ID NO:90; and
(d) a second heavy chain comprising the amino acid sequence of SEQ ID NO:91.

In one embodiment, provided is an immune activating fragment crystallizable (Fc) domain binding molecule comprising:
(a) a first light chain comprising an amino acid sequence of SEQ ID NO:89.
(b) a second light chain comprising the amino acid sequence of SEQ ID NO:70
(c) a first heavy chain comprising the amino acid sequence of SEQ ID NO:90; and
(d) a second heavy chain comprising the amino acid sequence of SEQ ID NO:91.

In one embodiment, provided is an immune activating fragment crystallizable (Fc) domain binding molecule comprising:
(a) a first light chain comprising an amino acid sequence of SEQ ID NO:89.
(b) a second light chain comprising the amino acid sequence of SEQ ID NO:70
(c) a first heavy chain comprising the amino acid sequence of SEQ ID NO:90; and
(d) a second heavy chain comprising the amino acid sequence of SEQ ID NO:92.

In one embodiment, provided is an immune activating fragment crystallizable (Fc) domain binding molecule comprising:
(a) a first light chain comprising an amino acid sequence of SEQ ID NO:93.
(b) a second light chain comprising the amino acid sequence of SEQ ID NO:68
(c) a first heavy chain comprising the amino acid sequence of SEQ ID NO:87; and
(d) a second heavy chain comprising the amino acid sequence of SEQ ID NO:88.

In one embodiment, provided is an immune activating fragment crystallizable (Fc) domain binding molecule comprising:
(a) a first light chain comprising an amino acid sequence that is at least about 95%, 96%, 97%, 98%, 99% or 100% identical to SEQ ID NO:89.
(b) a second light chain comprising the amino acid sequence that is at least about 95%, 96%, 97%, 98%, 99% or 100% identical to SEQ ID NO:70
(c) a first heavy chain comprising the amino acid sequence that is at least about 95%, 96%, 97%, 98%, 99% or 100% identical to SEQ ID NO:178; and
(d) a second heavy chain comprising the amino acid sequence that is at least about 95%, 96%, 97%, 98%, 99% or 100% identical to SEQ ID NO:179.

In one embodiment, provided is an immune activating fragment crystallizable (Fc) domain binding molecule comprising:
(a) a first light chain comprising an amino acid sequence that is at least about 95%, 96%, 97%, 98%, 99% or 100% identical to SEQ ID NO:89.
(b) a second light chain comprising the amino acid sequence that is at least about 95%, 96%, 97%, 98%, 99% or 100% identical to SEQ ID NO:68
(c) a first heavy chain comprising the amino acid sequence that is at least about 95%, 96%, 97%, 98%, 99% or 100% identical to SEQ ID NO:178; and
(d) a second heavy chain comprising the amino acid sequence that is at least about 95%, 96%, 97%, 98%, 99% or 100% identical to SEQ ID NO:179.

In one embodiment, provided is an immune activating fragment crystallizable (Fc) domain binding molecule comprising:
(a) a first light chain comprising an amino acid sequence that is at least about 95%, 96%, 97%, 98%, 99% or 100% identical to SEQ ID NO:89.
(b) a second light chain comprising the amino acid sequence that is at least about 95%, 96%, 97%, 98%, 99% or 100% identical to SEQ ID NO:180
(c) a first heavy chain comprising the amino acid sequence that is at least about 95%, 96%, 97%, 98%, 99% or 100% identical to SEQ ID NO:178; and
(d) a second heavy chain comprising the amino acid sequence that is at least about 95%, 96%, 97%, 98%, 99% or 100% identical to SEQ ID NO:179.

In one embodiment, provided is an immune activating fragment crystallizable (Fc) domain binding molecule comprising:
(a) a first light chain comprising an amino acid sequence of SEQ ID NO:89.
(b) a second light chain comprising the amino acid sequence of SEQ ID NO:70
(c) a first heavy chain comprising the amino acid sequence of SEQ ID NO:178; and
(d) a second heavy chain comprising the amino acid sequence of SEQ ID NO:179.

In one embodiment, provided is an immune activating fragment crystallizable (Fc) domain binding molecule comprising:
(a) a first light chain comprising an amino acid sequence of SEQ ID NO:89.
(b) a second light chain comprising the amino acid sequence of SEQ ID NO:68
(c) a first heavy chain comprising the amino acid sequence of SEQ ID NO:178; and
(d) a second heavy chain comprising the amino acid sequence of SEQ ID NO:179.

In one embodiment, provided is an immune activating fragment crystallizable (Fc) domain binding molecule comprising:
(a) a first light chain comprising an amino acid sequence of SEQ ID NO:89.
(b) a second light chain comprising the amino acid sequence of SEQ ID NO:180
(c) a first heavy chain comprising the amino acid sequence of SEQ ID NO:187; and
(d) a second heavy chain comprising the amino acid sequence of SEQ ID NO:179.

In a further embodiment, provided is any one of the immune activating fragment crystallizable (Fc) domain binding molecule as described herein above further comprising a substitution at position P329 (numbering according to Kabat EU index) by an amino acid selected from the list consisting of arginine (R), leucine (L), isoleucine (I), and alanine (A).

CD28 Binding Immune Activating Fc Domain Binding Molecules

In a particular embodiment according to the invention, the immune activating Fc domain binding molecule is capable of simultaneous binding to an Fc domain binding moiety which specifically binds to a target Fc domain comprising a first set of at least one amino acid substitution as herein above described, and a costimulatory T cell antigen, particularly CD28. The immune activating Fc domain binding molecule of the invention is combined with a targeting antibody comprising an Fc domain comprising the first set of at least one amino acid substitution and at least one antigen binding moiety capable of specific binding to an antigen on a target cell. In such embodiments, the immune activating Fc domain binding molecule is capable of crosslinking a T cell and a target cell by simultaneous binding to the target Fc domain and a costimulatory T cell antigen while the targeting antibody binds to the target cell. In an even more particular embodiment, such simultaneous binding results in lysis of the target cell, particularly a tumor cell. In one embodiment, such simultaneous binding results in activation or increased activation of the T cell. In other embodiments, such simultaneous binding results in a (increased) cellular response of a T lymphocyte, particularly a cytotoxic T lymphocyte, selected from the group of: proliferation, differentiation, cytokine secretion, cytotoxic effector molecule release, cytotoxic activity, and expression of activation markers. In one embodiment, binding of the immune activating Fc domain binding molecule to the costimulatory T cell antigen, particularly CD28, without simultaneous crosslinking to the target cell does not result in (increased) T cell activation.

In one embodiment, the immune activating Fc domain binding molecule in combination with the targeting antibody is capable of increasing cytotoxic activity of a T cell to a target cell. In a particular embodiment, said re-direction is independent of MHC-mediated peptide antigen presentation by the target cell and and/or specificity of the T cell.

Particularly, a T cell according to any of the embodiments of the invention is a cytotoxic T cell. In some embodiments the T cell is a CD4⁺ or a CD8⁺ T cell, particularly a CD8⁺ T cell.

Accordingly, in one aspect of the present invention, the immune activating moiety is an antigen binding moiety capable of specific binding to a costimulatory T cell antigen, in particular CD28. In one embodiment, the immune activating Fc domain binding molecule of the invention comprises at least one Fab molecule which specifically binds to the costimulatory T cell antigen (also referred to herein as an “costimulatory T cell antigen binding Fab molecule”). In a particular embodiment, the immune activating Fc domain binding molecule comprises not more than one Fab molecule (or other Fab molecule) capable of specific binding to a costimulatory T cell antigen.

In one embodiment the immune activating Fc domain binding molecule provides monovalent binding to the costimulatory T cell antigen.

In particular embodiments, the Fab molecule which specifically binds a costimulatory T cell antigen is a crossover Fab molecule as described herein, i.e. a Fab molecule wherein the variable domains VH and VL of the Fab heavy and light chains are exchanged/replaced by each other. In such embodiments, the Fab molecule(s) which specifically binds a target Fc domain comprising a first set of at least one amino acid substitution is a conventional Fab molecule. In embodiments where there is more than one Fab molecule which specifically binds to a target Fc domain comprised in the immune activating Fc domain binding molecule, the Fab molecule which specifically binds to a costimulatory T cell antigen preferably is a crossover Fab molecule and the Fab molecules which specifically bind to a target Fc domain are conventional Fab molecules.

In alternative embodiments, the Fab molecule which specifically binds a costimulatory T cell antigen is a conventional Fab molecule. In such embodiments, the Fab molecule(s) which specifically binds a target Fc domain is a crossover Fab molecule as described herein, i.e. a Fab molecule wherein the variable domains VH and VL of the Fab heavy and light chains are exchanged/replaced by each other.

In a particular embodiment the costimulatory T cell antigen is CD28, particularly human CD28. In a particular embodiment the costimulatory T cell antigen binding Fab molecule is cross-reactive for (i.e. specifically binds to) human and cynomolgus CD28.

In some embodiments, the costimulatory T cell antigen binding Fab molecule specifically binds to CD28 and comprises at least one heavy chain complementarity determining region (CDR) selected from the group consisting of SEQ ID NO: 94, SEQ ID NO: 95 and SEQ ID NO: 96 and at least one light chain CDR selected from the group of SEQ ID NO: 97, SEQ ID NO: 98, SEQ ID NO: 99.

In one embodiment the CD28 binding Fab molecule comprises a heavy chain variable region comprising the heavy chain CDR1 of SEQ ID NO: 94, the heavy chain CDR2 of SEQ ID NO: 95, the heavy chain CDR3 of SEQ ID NO: 96, and a light chain variable region comprising the light chain CDR1 of SEQ ID NO: 97, the light chain CDR2 of SEQ ID NO: 98, and the light chain CDR3 of SEQ ID NO: 99.

In some embodiments, the costimulatory T cell antigen binding Fab molecule specifically binds to CD28 and comprises at least one heavy chain complementarity determining region (CDR) selected from the group consisting of SEQ ID NO: 94, SEQ ID NO: 95 and SEQ ID NO: 102 and at least one light chain CDR selected from the group of SEQ ID NO: 103, SEQ ID NO: 98, SEQ ID NO: 99.

In another embodiment the CD28 binding Fab molecule comprises a heavy chain variable region comprising the heavy chain CDR1 of SEQ ID NO: 94, the heavy chain CDR2 of SEQ ID NO: 95, the heavy chain CDR3 of SEQ ID NO: 102, and a light chain variable region comprising the light chain CDR1 of SEQ ID NO: 103, the light chain CDR2 of SEQ ID NO: 98, and the light chain CDR3 of SEQ ID NO: 99.

In one embodiment the CD28 binding Fab molecule comprises a heavy chain variable region sequence that is at least about 95%, 96%, 97%, 98%, 99% or 100% identical to SEQ ID NO: 100 and a light chain variable region sequence that is at least about 95%, 96%, 97%, 98%, 99% or 100% identical to SEQ ID NO: 101.

In one embodiment the CD28 binding Fab molecule comprises a heavy chain variable region comprising the amino acid sequence of SEQ ID NO: 100 and a light chain variable region comprising the amino acid sequence of SEQ ID NO: 101.

In one embodiment the CD28 binding Fab molecule comprises the heavy chain variable region sequence of SEQ ID NO: 104 and the light chain variable region sequence of SEQ ID NO: 105.

In one embodiment the CD28 binding Fab molecule comprises a heavy chain variable region sequence that is at least about 95%, 96%, 97%, 98%, 99% or 100% identical to SEQ ID NO: 104 and a light chain variable region sequence that is at least about 95%, 96%, 97%, 98%, 99% or 100% identical to SEQ ID NO: 105.

In one embodiment the CD28 binding Fab molecule comprises a heavy chain variable region comprising the amino acid sequence of SEQ ID NO: 104 and a light chain variable region comprising the amino acid sequence of SEQ ID NO: 105.

In one embodiment the CD28 binding Fab molecule comprises the heavy chain variable region sequence of SEQ ID NO: 104 and the light chain variable region sequence of SEQ ID NO: 105.

In one embodiment, provided is an immune activating fragment crystallizable (Fc) domain binding molecule comprising:
(a) a first light chain comprising an amino acid sequence of SEQ ID NO:93.
(b) a second light chain comprising the amino acid sequence of SEQ ID NO:106
(c) a first heavy chain comprising the amino acid sequence of SEQ ID NO:88; and
(d) a second heavy chain comprising the amino acid sequence of SEQ ID NO:107.

In one embodiment, provided is an immune activating fragment crystallizable (Fc) domain binding molecule comprising:
(a) a first light chain comprising an amino acid sequence of SEQ ID NO:93.
(b) a second light chain comprising the amino acid sequence of SEQ ID NO:108
(c) a first heavy chain comprising the amino acid sequence of SEQ ID NO:88; and
(d) a second heavy chain comprising the amino acid sequence of SEQ ID NO:109.

In one embodiment, provided is an immune activating fragment crystallizable (Fc) domain binding molecule comprising:
(a) a first light chain comprising an amino acid sequence of SEQ ID NO:89.
(b) a second light chain comprising the amino acid sequence of SEQ ID NO:108
(c) a first heavy chain comprising the amino acid sequence of SEQ ID NO:90; and
(d) a second heavy chain comprising the amino acid sequence of SEQ ID NO:109.

In one embodiment, provided is an immune activating fragment crystallizable (Fc) domain binding molecule comprising:
(a) a first light chain comprising an amino acid sequence of SEQ ID NO:110.
(b) a second light chain comprising the amino acid sequence of SEQ ID NO:111
(c) a first heavy chain comprising the amino acid sequence of SEQ ID NO:112; and
(d) a second heavy chain comprising the amino acid sequence of SEQ ID NO:113.

In one embodiment, provided is an immune activating fragment crystallizable (Fc) domain binding molecule comprising:
(a) a first light chain comprising an amino acid sequence of SEQ ID NO:89.
(b) a second light chain comprising the amino acid sequence of SEQ ID NO:108
(c) a first heavy chain comprising the amino acid sequence of SEQ ID NO:90; and
(d) a second heavy chain comprising the amino acid sequence of SEQ ID NO:114.

In one embodiment, provided is an immune activating fragment crystallizable (Fc) domain binding molecule comprising:
(a) a first light chain comprising an amino acid sequence of SEQ ID NO:89.
(b) a second light chain comprising the amino acid sequence of SEQ ID NO:108
(c) a first heavy chain comprising the amino acid sequence of SEQ ID NO:90; and
(d) a second heavy chain comprising the amino acid sequence of SEQ ID NO:115.

In a further embodiment, provided is any one of the immune activating fragment crystallizable (Fc) domain binding molecule as described herein above further comprising a substitution at position P329 (numbering according to Kabat EU index) by an amino acid selected from the list consisting of arginine (R), leucine (L), isoleucine (I), and alanine (A).

4-1BB Binding Immune Activating Fc Domain Binding Molecules

In a particular embodiment according to the invention, the immune activating Fc domain binding molecule is capable of simultaneous binding to an Fc domain binding moiety which specifically binds to a target Fc domain comprising a first set of at least one amino acid substitution as herein above described, and to 4-1BB. Accordingly, in one aspect of the present invention, the immune activating moiety is an antigen binding moiety capable of specific binding to a costimulatory T cell antigen, in particular 4-1BB. The immune activating Fc domain binding molecule of the invention is combined with a targeting antibody comprising an Fc domain comprising the first set of at least one amino acid substitution and at least one antigen binding moiety capable of specific binding to an antigen on a target cell. In such embodiments, the immune activating Fc domain binding molecule is capable of crosslinking a T cell and a target cell by simultaneous binding to the target Fc domain and a costimulatory T cell antigen while the targeting antibody binds to the target cell. In an even more particular embodiment, such simultaneous binding results in lysis of the target cell, particularly a tumor cell. In one embodiment, such simultaneous binding results in activation or increased activation of the T cell. In other embodiments, such simultaneous binding results in a (increased) cellular response of a T lymphocyte, particularly a cytotoxic T lymphocyte, selected from the group of: proliferation, differentiation, cytokine secretion, cytotoxic effector molecule release, cytotoxic activity, and expression of activation markers. In one embodiment, binding of the immune activating Fc domain binding molecule to the costimulatory T cell antigen, particularly 4-1BB, without simultaneous crosslinking to the target cell does not result in (increased) T cell activation.

In one embodiment, the immune activating Fc domain binding molecule in combination with the targeting antibody is capable of increasing cytotoxic activity of a T cell to a target cell. In a particular embodiment, said re-direction is independent of MHC-mediated peptide antigen presentation by the target cell and and/or specificity of the T cell.

Particularly, a T cell according to any of the embodiments of the invention is a cytotoxic T cell. In some embodiments the T cell is a CD4⁺ or a CD8⁺ T cell, particularly a CD8⁺ T cell.

In one embodiment, the immune activating Fc domain binding molecule of the invention comprises at least one Fab molecule which specifically binds to the costimulatory T cell antigen (also referred to herein as an “costimulatory T cell antigen binding Fab molecule”). In a particular embodiment, the immune activating Fc domain binding molecule comprises not more than one Fab molecule (or other Fab molecule) capable of specific binding to a costimulatory T cell antigen.

In one embodiment the immune activating Fc domain binding molecule provides monovalent binding to the costimulatory antigen.

In particular embodiments, the Fab molecule which specifically binds a costimulatory T cell antigen is a crossover Fab molecule as described herein, i.e. a Fab molecule wherein the variable domains VH and VL of the Fab heavy and light chains are exchanged/replaced by each other. In such embodiments, the Fab molecule(s) which specifically binds a target Fc domain comprising a first set of at least one amino acid substitution is a conventional Fab molecule. In embodiments where there is more than one Fab molecule which specifically binds to a target Fc domain comprised in the immune activating Fc domain binding molecule, the Fab molecule which specifically binds to a costimulatory T cell antigen preferably is a crossover Fab molecule and the Fab molecules which specifically bind to a target Fc domain are conventional Fab molecules.

In alternative embodiments, the Fab molecule which specifically binds a costimulatory T cell antigen is a conventional Fab molecule. In such embodiments, the Fab molecule(s) which specifically binds a target Fc domain is a crossover Fab molecule as described herein, i.e. a Fab molecule wherein the variable domains VH and VL of the Fab heavy and light chains are exchanged/replaced by each other.

In a particular embodiment the costimulatory T cell antigen is 4-1BB, particularly human CD28.

In a particular embodiment the costimulatory T cell antigen binding Fab molecule is cross-reactive for (i.e. specifically binds to) human and cynomolgus 4-1BB.

In some embodiments, the costimulatory T cell antigen binding Fab molecule specifically binds to 4-1BB and comprises at least one heavy chain complementarity determining region (CDR) selected from the group consisting of SEQ ID NO: 133, SEQ ID NO: 134 and SEQ ID NO: 135 and at least one light chain CDR selected from the group of SEQ ID NO: 136, SEQ ID NO: 137, SEQ ID NO: 138.

In one embodiment the 4-1BB binding Fab molecule comprises a heavy chain variable region comprising the heavy chain CDR1 of SEQ ID NO: 133, the heavy chain CDR2 of SEQ ID NO: 134, the heavy chain CDR3 of SEQ ID NO: 135, and a light chain variable region comprising the light chain CDR1 of SEQ ID NO: 136, the light chain CDR2 of SEQ ID NO: 137, and the light chain CDR3 of SEQ ID NO: 138

In one embodiment the 4-1BB binding Fab molecule comprises a heavy chain variable region sequence that is at least about 95%, 96%, 97%, 98%, 99% or 100% identical to SEQ ID NO: 139 and a light chain variable region sequence that is at least about 95%, 96%, 97%, 98%, 99% or 100% identical to SEQ ID NO: 140.

In one embodiment the 4-1BB binding Fab molecule comprises a heavy chain variable region comprising the amino acid sequence of SEQ ID NO: 139 and a light chain variable region comprising the amino acid sequence of SEQ ID NO: 140.

In one embodiment the CD28 binding Fab molecule comprises the heavy chain variable region sequence of SEQ ID NO: 139 and the light chain variable region sequence of SEQ ID NO: 140.

In one embodiment provided is an immune activating fragment crystallizable (Fc) domain binding molecule comprising:
(a) a first light chain comprising an amino acid sequence of SEQ ID NO:141.
(b) a second light chain comprising the amino acid sequence of SEQ ID NO:142
(c) a first heavy chain comprising the amino acid sequence of SEQ ID NO:143; and
(d) a second heavy chain comprising the amino acid sequence of SEQ ID NO:144.

In one embodiment provided is an immune activating fragment crystallizable (Fc) domain binding molecule comprising:
(a) a first light chain comprising an amino acid sequence of SEQ ID NO:110.
(b) a second light chain comprising the amino acid sequence of SEQ ID NO:142
(c) a first heavy chain comprising the amino acid sequence of SEQ ID NO:145; and
(d) a second heavy chain comprising the amino acid sequence of SEQ ID NO:144.

In a further embodiment, provided is any one of the immune activating fragment crystallizable (Fc) domain binding molecule as described herein above further comprising a substitution at position P329 (numbering according to Kabat EU index) by an amino acid selected from the list consisting of arginine (R), leucine (L), isoleucine (I), and alanine (A).

Immune Activating Fc Domain Binding Molecules Comprising a Cytokine

In one embodiment, the immune activating moiety is a cytokine. In one embodiment, the cytokine is selected from the group consisting of IL2, IL7, IL15, IL18, IFNa and IFNg. In a particular such embodiment, the immune activating fragment crystallizable (Fc) domain binding molecule of the invention comprises a mutant IL-2 polypeptide having advantageous properties for immunotherapy. In particular, pharmacological properties of IL-2 that contribute to toxicity but are not essential for efficacy of IL-2 are eliminated in the mutant IL-2 polypeptide. Such mutant IL-2 polypeptides are described in detail in WO 2012/107417, which is incorporated herein by reference in its entirety. As discussed above, different forms of the IL-2 receptor consist of different subunits and exhibit different affinities for IL-2. The intermediate-affinity IL-2 receptor, consisting of the β and γ receptor subunits, is expressed on resting effector cells and is sufficient for IL-2 signaling. The high-affinity IL-2 receptor, additionally comprising the α-subunit of the receptor, is mainly expressed on regulatory T (T_{reg}) cells as well as on activated effector cells where its engagement by IL-2 can promote T_{reg} cell-mediated immunosuppression or activation-induced cell death (AICD), respectively. Thus, without wishing to be bound by theory, reducing or abolishing the affinity of IL-2 to the α-subunit of the IL-2 receptor should reduce IL-2 induced downregulation of effector cell function by regulatory T cells and development of tumor tolerance by the process of AICD. On the other hand, maintaining the affinity to the intermediate-affinity IL-2 receptor should preserve the induction of proliferation and activation of effector cells like NK and T cells by IL-2.

The mutant interleukin-2 (IL-2) polypeptide comprised in the immune activating fragment crystallizable (Fc) domain binding molecule according to the invention comprises at least one amino acid mutation that abolishes or reduces affinity of the mutant IL-2 polypeptide to the α-subunit of the IL-2 receptor and preserves affinity of the mutant IL-2 polypeptide to the intermediate-affinity IL-2 receptor each compared to a wild-type IL-2 polypeptide.

Mutants of human IL-2 (hIL-2) with decreased affinity to CD25 may for example be generated by amino acid substitution at amino acid position 35, 38, 42, 43, 45 or 72 or combinations thereof (numbering relative to the human IL-2 sequence SEQ ID NO: 166). Exemplary amino acid substitutions include K35E, K35A, R38A, R38E, R38N, R38F, R38S, R38L, R38G, R38Y, R38W, F42L, F42A, F42G, F42S, F42T, F42Q, F42E, F42N, F42D, F42R, F42K, K43E, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K. Particular IL-2 mutants useful in the immune activating fragment crystallizable (Fc) domain binding molecule of the invention comprise an amino acid mutation at an amino acid position corresponding to residue 42, 45, or 72 of human IL-2, or a combination thereof. In one embodiment said amino acid mutation is an amino acid substitution selected from the group of F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K, more specifically an amino acid substitution selected from the group of F42A, Y45A and L72G. These mutants exhibit substantially similar binding affinity to the intermediate-affinity IL-2 receptor, and have substantially reduced affinity to the α-subunit of the IL-2 receptor and the high-affinity IL-2 receptor compared to a wild-type form of the IL-2 mutant.

Other characteristics of useful mutants may include the ability to induce proliferation of IL-2 receptor-bearing T and/or NK cells, the ability to induce IL-2 signaling in IL-2 receptor-bearing T and/or NK cells, the ability to generate interferon (IFN)-γ as a secondary cytokine by NK cells, a reduced ability to induce elaboration of secondary cytokines—particularly IL-10 and TNF-α—by peripheral blood mononuclear cells (PBMCs), a reduced ability to activate regulatory T cells, a reduced ability to induce apoptosis in T cells, and a reduced toxicity profile in vivo.

Particular mutant IL-2 polypeptides useful in the invention comprise three amino acid mutations that abolish or reduce affinity of the mutant IL-2 polypeptide to the α-subunit of the IL-2 receptor but preserve affinity of the mutant IL-2 polypeptide to the intermediate affinity IL-2 receptor. In one embodiment said three amino acid mutations are at positions corresponding to residue 42, 45 and 72 of human IL-2. In one embodiment said three amino acid mutations are amino acid substitutions. In one embodiment said three amino acid mutations are amino acid substitutions selected from the group of F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K. In a specific embodiment said three amino acid mutations are amino acid substitutions F42A, Y45A and L72G (numbering relative to the human IL-2 sequence of SEQ ID NO: 166).

In certain embodiments said amino acid mutation reduces the affinity of the mutant IL-2 polypeptide to the α-subunit of the IL-2 receptor by at least 5-fold, specifically at least 10-fold, more specifically at least 25-fold. In embodiments where there is more than one amino acid mutation that reduces the affinity of the mutant IL-2 polypeptide to the α-subunit of the IL-2 receptor, the combination of these amino acid mutations may reduce the affinity of the mutant IL-2 polypeptide to the α-subunit of the IL-2 receptor by at least 30-fold, at least 50-fold, or even at least 100-fold. In one embodiment said amino acid mutation or combination of amino acid mutations abolishes the affinity of the mutant IL-2 polypeptide to the α-subunit of the IL-2 receptor so that no binding is detectable by surface plasmon resonance.

Substantially similar binding to the intermediate-affinity receptor, i.e. preservation of the affinity of the mutant IL-2 polypeptide to said receptor, is achieved when the IL-2 mutant exhibits greater than about 70% of the affinity of a wild-type form of the IL-2 mutant to the intermediate-affinity IL-2 receptor. IL-2 mutants of the invention may exhibit greater than about 80% and even greater than about 90% of such affinity.

Reduction of the affinity of IL-2 for the α-subunit of the IL-2 receptor in combination with elimination of the O-glycosylation of IL-2 results in an IL-2 protein with improved properties. For example, elimination of the O-glycosylation site results in a more homogenous product when the mutant IL-2 polypeptide is expressed in mammalian cells such as CHO or HEK cells.

Thus, in certain embodiments the mutant IL-2 polypeptide comprises an additional amino acid mutation which eliminates the O-glycosylation site of IL-2 at a position corresponding to residue 3 of human IL-2. In one embodiment said additional amino acid mutation which eliminates the O-glycosylation site of IL-2 at a position corresponding to residue 3 of human IL-2 is an amino acid substitution. Exemplary amino acid substitutions include T3A, T3G, T3Q, T3E, T3N, T3D, T3R, T3K, and T3P. In a specific embodiment, said additional amino acid mutation is the amino acid substitution T3A.

In certain embodiments the mutant IL-2 polypeptide is essentially a full-length IL-2 molecule. In certain embodiments the mutant IL-2 polypeptide is a human IL-2 molecule. In one embodiment the mutant IL-2 polypeptide comprises the sequence of SEQ ID NO: 166 with at least one amino acid mutation that abolishes or reduces affinity of the mutant IL-2 polypeptide to the α-subunit of the IL-2 receptor but preserve affinity of the mutant IL-2 polypeptide to the intermediate affinity IL-2 receptor, compared to an IL-2 polypeptide comprising SEQ ID NO: 166 without said mutation. In another embodiment, the mutant IL-2 polypeptide comprises the sequence of SEQ ID NO: 167 with at least one amino acid mutation that abolishes or reduces affinity of the mutant IL-2 polypeptide to the α-subunit of the IL-2 receptor but preserve affinity of the mutant IL-2 polypeptide to the intermediate affinity IL-2 receptor, compared to an IL-2 polypeptide comprising SEQ ID NO: 167 without said mutation.

In a specific embodiment, the mutant IL-2 polypeptide can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

In one embodiment the mutant IL-2 polypeptide has a reduced ability to induce IL-2 signaling in regulatory T cells, compared to a wild-type IL-2 polypeptide. In one embodiment the mutant IL-2 polypeptide induces less activation-induced cell death (AICD) in T cells, compared to a wild-type IL-2 polypeptide. In one embodiment the mutant IL-2 polypeptide has a reduced toxicity profile in vivo, compared to a wild-type IL-2 polypeptide. In one embodiment the mutant IL-2 polypeptide has a prolonged serum half-life, compared to a wild-type IL-2 polypeptide.

A particular mutant IL-2 polypeptide useful in the invention comprises four amino acid substitutions at positions corresponding to residues 3, 42, 45 and 72 of human IL-2. Specific amino acid substitutions are T3A, F42A, Y45A and L72G. As demonstrated in WO 2012/107417, said quadruple mutant IL-2 polypeptide exhibits no detectable binding to CD25, reduced ability to induce apoptosis in T cells, reduced ability to induce IL-2 signaling in T_{reg} cells, and a reduced toxicity profile in vivo. However, it retains ability to activate IL-2 signaling in effector cells, to induce proliferation of effector cells, and to generate IFN-γ as a secondary cytokine by NK cells. Moreover, said mutant IL-2 polypeptide has further advantageous properties, such as reduced surface hydrophobicity, good stability, and good expression yield, as described in WO 2012/107417. Unexpectedly, said mutant IL-2 polypeptide also provides a prolonged serum half-life, compared to wild-type IL-2.

IL-2 mutants useful in the invention, in addition to having mutations in the region of IL-2 that forms the interface of IL-2 with CD25 or the glycosylation site, also may have one or more mutations in the amino acid sequence outside these regions. Such additional mutations in human IL-2 may provide additional advantages such as increased expression or stability. For example, the cysteine at position 125 may be replaced with a neutral amino acid such as serine, alanine, threonine or valine, yielding C125S IL-2, C125A IL-2, C125T IL-2 or C125V IL-2 respectively, as described in U.S. Pat. No. 4,518,584. As described therein, one may also delete the N-terminal alanine residue of IL-2 yielding such mutants as des-A1 C125S or des-A1 C125A. Alternatively or conjunctively, the IL-2 mutant may include a mutation whereby methionine normally occurring at position 104 of wild-type human IL-2 is replaced by a neutral amino acid such as alanine (see U.S. Pat. No. 5,206,344). The resulting mutants, e. g., des-A1 M104A IL-2, des-A1 M104A C125S IL-2, M104A IL-2, M104A C125A IL-2, des-A1 M104A C125A IL-2, or M104A C125S IL-2 (these and other mutants may be found in U.S. Pat. No. 5,116,943 and in Weiger et al., Eur J Biochem 180, 295-300 (1989)) may be used in conjunction with the particular IL-2 mutations of the invention.

Thus, in certain embodiments the mutant IL-2 polypeptide comprises an additional amino acid mutation at a position corresponding to residue 125 of human IL-2. In one embodiment said additional amino acid mutation is the amino acid substitution C125A.

The skilled person will be able to determine which additional mutations may provide additional advantages for the purpose of the invention. For example, he will appreciate that amino acid mutations in the IL-2 sequence that reduce or abolish the affinity of IL-2 to the intermediate-affinity IL-2 receptor, such as D20T, N88R or Q126D (see e.g. US 2007/0036752), may not be suitable to include in the mutant IL-2 polypeptide according to the invention.

In one embodiment, the mutant IL-2 polypeptide comprises no more than 12, no more than 11, no more than 10, no more than 9, no more than 8, no more than 7, no more than 6, or no more than 5 amino acid mutations as compared to the corresponding wild-type IL-2 sequence, e.g. the human IL-2 sequence of SEQ ID NO: 166. In a particular embodiment, the mutant IL-2 polypeptide comprises no more than 5 amino acid mutations as compared to the corresponding wild-type IL-2 sequence, e.g. the human IL-2 sequence of SEQ ID NO: 166.

In one embodiment the mutant IL-2 polypeptide comprises the sequence of SEQ ID NO: 167. In one embodiment the mutant IL-2 polypeptide consists of the sequence of SEQ ID NO: 167.

In one aspect, the invention provides an immune activating fragment crystallizable (Fc) domain binding molecule comprising a mutant IL-2 comprising
(a) an Fc domain binding moiety which specifically binds to a target Fc domain comprising a first set of at least one amino acid substitution as herein described,
(b) an immuoactivating moiety which is a mutant IL-2 polypeptide, wherein the mutant IL-2 polypeptide is a human IL-2 molecule comprising the amino acid substitutions F42A, Y45A and L72G (numbering relative to the human IL-2 sequence SEQ ID NO: 166); and
(c) a half-life extending Fc domain composed of a first and a second subunit capable of stable association as herein described,
wherein the Fc domain binding moiety does not specifically bind to the half-life extending Fc domain.

In one aspect, the invention provides an immune activating fragment crystallizable (Fc) domain binding molecule comprising a mutant IL-2 comprising
(a) an Fc domain binding moiety which specifically binds to a target Fc domain comprising a first set of at least one amino acid substitution as herein described,
(b) an immuoactivating moiety which is a mutant IL-2 polypeptide, wherein the mutant IL-2 polypeptide is a human IL-2 molecule comprising the amino acid substitutions T3A, F42A, Y45A, L72G and C125A (numbering relative to the human IL-2 sequence SEQ ID NO: 166); and
(c) a half-life extending Fc domain composed of a first and a second subunit capable of stable association as herein described,
wherein the Fc domain binding moiety does not specifically bind to the half-life extending Fc domain.

In one aspect, the invention provides an immune activating fragment crystallizable (Fc) domain binding molecule comprising a mutant IL-2 comprising
(a) an Fc domain binding moiety which specifically binds to a target Fc domain comprising a first set of at least one amino acid substitution as herein described,
(b) an immuoactivating moiety which is a mutant IL-2 polypeptide, wherein the mutant IL-2 polypeptide comprises the amino acid sequence of SEQ ID NO: 167; and
(c) a half-life extending Fc domain composed of a first and a second subunit capable of stable association as herein described,
wherein the Fc domain binding moiety does not specifically bind to the half-life extending Fc domain.

In any of the above embodiments, the mutant IL-2 polypeptide may be fused at its amino-terminal amino acid to the carboxy-terminal amino acid of the one or both subunits of the half-life extending Fc domain, through a linker peptide

In one aspect, provided is an immune activating fragment crystallizable (Fc) domain binding molecule comprising:
(a) a light chain comprising an amino acid sequence of SEQ ID NO:86.
(c) a first heavy chain comprising the amino acid sequence of SEQ ID NO:116; and
(d) a second heavy chain comprising the amino acid sequence of SEQ ID NO:88.

In one aspect, provided is an immune activating fragment crystallizable (Fc) domain binding molecule comprising:
(a) a light chain comprising an amino acid sequence of SEQ ID NO:15.
(c) a first heavy chain comprising the amino acid sequence of SEQ ID NO:116; and
(d) a second heavy chain comprising the amino acid sequence of SEQ ID NO:90.

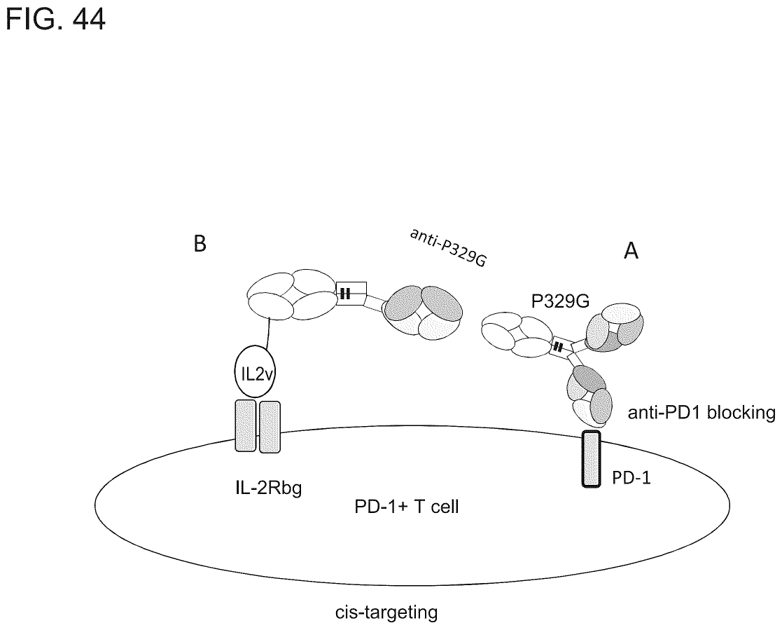

In a preferred aspect, the immune activating fragment crystallizable (Fc) domain binding molecule is combined with a targeting antibody capable of specific binding to a T cell antigen, in particular CD8 or PD-1. In a preferred embodiment targeting antibody is capable of specific binding to PD-1. Such combination are useful for cis-activation of T cells (see FIG. 44) In a particular embodiment, the immune activating fragment crystallizable (Fc) domain binding molecule is combined with a targeting antibody comprising a first light chain comprising an amino acid sequence of SEQ ID NO:160. and a heavy chain comprising the amino acid sequence of SEQ ID NO:161.

In a further embodiment, provided is any one of the immune activating fragment crystallizable (Fc) domain binding molecule as described herein above further comprising a substitution at position P329 (numbering according to Kabat EU index) by an amino acid selected from the list consisting of arginine (R), leucine (L), isoleucine (I), and alanine (A).

4-1BBL Trimer-Containing Immune Activating Fc Domain Binding Molecules

In one aspect of the present invention, the immune activating moiety is a costimulatory T cell ligand, in particular 4-1BBL. Accordingly, in another aspect, the invention also provides novel 4-1BBL trimer-containing immune activating Fc domain binding molecules.

In a first aspect, the invention provides an immune activating fragment crystallizable (Fc) domain binding molecule comprising
(a) an Fc domain binding moiety which specifically binds to a target Fc domain as herein described,
(b) a first and a second polypeptide that are linked to each other by a disulfide bond, wherein the immune activating Fc domain binding molecule is characterized in that the first polypeptide comprises two ectodomains of 4-1BBL or a fragment thereof that are connected to each other by a peptide linker and in that the second polypeptide comprises one ectodomain of 4-1BBL or a fragment thereof, and
(c) a half-life extending Fc domain composed of a first and a second subunit capable of stable association as herein described,
wherein the Fc domain binding moiety does not specifically bind to the half-life extending Fc domain.

In a further aspect, provided is an immune activating fragment crystallizable (Fc) domain binding molecule as defined herein before, comprising
(a) an Fc domain binding moiety which specifically binds to a target Fc domain as herein described, and
(b) a first and a second polypeptide that are linked to each other by a disulfide bond, wherein the immune activating Fc domain binding molecule is characterized in that
(i) the first polypeptide contains a CH1 or CL domain and the second polypeptide contains a CL or CH1 domain, respectively, wherein the second polypeptide is linked to the first polypeptide by a disulfide bond between the CH1 and CL domain, and wherein the first polypeptide comprises two ectodomains of 4-1BBL or a fragment thereof that are connected to each other and to the CH1 or CL domain by a peptide linker and wherein the second polypeptide comprises one ectodomain of said 4-1BBL or a fragment thereof connected via a peptide linker to the CL or CH1 domain of said polypeptide, or
(ii) the first polypeptide contains a CH3 domain and the second polypeptide contains a CH3 domain, respectively, and wherein the first polypeptide comprises two ectodomains of a 4-1BBL or a fragment thereof that are connected to each other and to the C-terminus of the CH3 domain by a peptide linker and wherein the second polypeptide comprises only one ectodomain of said 4-1BBL or a fragment thereof connected via a peptide linker to C-terminus of the CH3 domain of said polypeptide, or
(iii) the first polypeptide contains a VH-CL or a VL-CH1 domain and the second polypeptide contains a VL-CH1 domain or a VH-CL domain, respectively, wherein the second polypeptide is linked to the first polypeptide by a disulfide bond between the CH1 and CL domain, and wherein the first polypeptide comprises two ectodomains of 4-1BBL or a fragment thereof that are connected to each other and to to VH or VL by a peptide linker and wherein the second polypeptide comprises one ectodomain of said TNF ligand family member or a fragment thereof connected via a peptide linker to VL or VH of said polypeptide, and
(c) a half-life extending Fc domain composed of a first and a second subunit capable of stable association as herein described,
wherein the Fc domain binding moiety does not specifically bind to the half-life extending Fc domain.

In another aspect, provided is an immune activating fragment crystallizable (Fc) domain binding molecule of as defined herein before, comprising
(a) an Fc domain binding moiety which specifically binds to a target Fc domain as herein described, and
(b) a first and a second polypeptide that are linked to each other by a disulfide bond, wherein the immune activating Fc domain binding molecule is characterized in that
(i) the first polypeptide contains a CH1 or CL domain and the second polypeptide contains a CL or CH1 domain, respectively, wherein the second polypeptide is linked to the first polypeptide by a disulfide bond between the CH1 and CL domain, and wherein the first polypeptide comprises two ectodomains of 4-1BBL or a fragment thereof that are connected to each other and to the CH1 or CL domain by a peptide linker and wherein the second polypeptide comprises one ectodomain of said 4-1BBL or a fragment thereof connected via a peptide linker to the CL or CH1 domain of said polypeptide, or
(ii) the first polypeptide contains a CH3 domain and the second polypeptide contains a CH3 domain, respectively, and wherein the first polypeptide comprises two ectodomains of 4-1BBL or a fragment thereof that are connected to each other and to the C-terminus of the CH3 domain by a peptide linker and wherein the second polypeptide comprises only one ectodomain of said 4-1BBL or a fragment thereof connected via a peptide linker to C-terminus of the CH3 domain of said polypeptide, and
(c) a half-life extending Fc domain composed of a first and a second subunit capable of stable association as herein described,
wherein the Fc domain binding moiety does not specifically bind to the half-life extending Fc domain.

In one aspect, the ectodomain of 4-1BBL comprises the amino acid sequence selected from the group consisting of SEQ ID NO:117, SEQ ID NO: 118, SEQ ID NO:119, SEQ ID NO:120, SEQ ID NO:121, SEQ ID NO: 122, SEQ ID NO:123 and SEQ ID NO:124, particularly the amino acid sequence of SEQ ID NO:117 or SEQ ID NO:121. More particularly, the ectodomain of 4-1BBL comprises the amino acid sequence of SEQ ID NO:117 or SEQ ID NO:121. Most particularly, the ectodomain of 4-1BBL comprises the amino acid sequence of SEQ ID NO:121. In particular, provided is an immune activating fragment crystallizable (Fc) domain binding molecule as defined herein before, wherein all three ectodomains of 4-1BBL or a fragment thereof are identical.

In a further aspect, the immune activating fragment crystallizable (Fc) domain binding molecule of the invention comprises
(a) an Fc domain binding moiety which specifically binds to a target Fc domain as herein described,
(b) a first and a second polypeptide that are linked to each other by a disulfide bond, wherein the antigen binding molecule is characterized in that the first polypeptide comprises the amino acid sequence selected from the group consisting of SEQ ID NO:125, SEQ ID NO:126, SEQ ID NO:127 and SEQ ID NO:128 and in that the second polypeptide comprises the amino acid sequence selected from the group consisting of SEQ ID NO:117, SEQ ID NO:121, SEQ ID NO:119 and SEQ ID NO:120, and
(c) a half-life extending Fc domain composed of a first and a second subunit capable of stable association as herein described, wherein the Fc domain binding moiety does not specifically bind to the half-life extending Fc domain.

In one aspect, the immune activating fragment crystallizable (Fc) domain binding molecule of the invention comprises
(a) an Fc domain binding moiety which specifically binds to a target Fc domain as herein described,
(b) a first and a second polypeptide that are linked to each other by a disulfide bond, wherein the antigen binding molecule is characterized in that the first polypeptide comprises the amino acid sequence of SEQ ID NO:126 and in that the second polypeptide comprises the amino acid sequence of SEQ ID NO:121, and
(c) a half-life extending Fc domain composed of a first and a second subunit capable of stable association as herein described,
wherein the Fc domain binding moiety does not specifically bind to the half-life extending Fc domain.

In a further aspect, the immune activating fragment crystallizable (Fc) domain binding molecule of the invention comprises
(a) an Fc domain binding moiety which specifically binds to a target Fc domain as herein described,
(b) a first and a second polypeptide that are linked to each other by a disulfide bond, wherein the antigen binding molecule is characterized in that the first polypeptide comprises the amino acid sequence of SEQ ID NO:125 and in that the second polypeptide comprises the amino acid sequence of SEQ ID NO:117, and
(c) a half-life extending Fc domain composed of a first and a second subunit capable of stable association as herein described,
wherein the Fc domain binding moiety does not specifically bind to the half-life extending Fc domain.

In another aspect, the immune activating fragment crystallizable (Fc) domain binding molecule of the invention comprises
(a) an Fc domain binding moiety which specifically binds to a target Fc domain as herein described,
(b) a first polypeptide containing a CH1 or CL domain and a second polypeptide containing a CL or CH1 domain, respectively, wherein the second polypeptide is linked to the first polypeptide by a disulfide bond between the CH1 and CL domain,
and wherein the antigen binding molecule is characterized in that the first polypeptide comprises two ectodomains of 4-1BBL or fragments thereof that are connected to each other and to the CH1 or CL domain by a peptide linker and in that the second polypeptide comprises only one ectodomain of 4-1BBL or a fragment thereof connected by a peptide linker to the CL or CH1 domain of said polypeptide.

In one aspect, provided is a immune activating fragment crystallizable (Fc) domain binding molecule comprising
(a) an Fc domain binding moiety which specifically binds to a target Fc domain as herein described,
(b) a first polypeptide containing a CH1 domain and a second polypeptide containing a CL domain, wherein the second polypeptide is linked to the first polypeptide by a disulfide bond between the CH1 and CL domain, and wherein the antigen binding molecule is characterized in that the first polypeptide comprises two ectodomains of 4-1BBL or a fragment thereof that are connected to each other and to the CH1 domain by a peptide linker and in that the second polypeptide comprises one ectodomain of 4-1BBL or a fragment thereof connected via a peptide linker to the CL domain of said polypeptide.

In another aspect, the invention provides an immune activating fragment crystallizable (Fc) domain binding molecule comprising
(a) an Fc domain binding moiety which specifically binds to a target Fc domain as herein described, and
(b) a first and a second polypeptide that are linked to each other by a disulfide bond,
wherein the antigen binding molecule is characterized in that the first polypeptide comprises two ectodomains of 4-1BBL or a fragment thereof that are connected to each other by a peptide linker and in that the second polypeptide comprises one ectodomain of 4-1BBL or a fragment thereof, and
(c) a half-life extending Fc domain composed of a first and a second subunit capable of stable association as herein described,
wherein the Fc domain binding moiety does not specifically bind to the half-life extending Fc domain.

In yet another aspect, the invention provides an immune activating fragment crystallizable (Fc) domain binding molecule comprising
(a) more than one Fc domain binding moiety which specifically binds to a target Fc domain as herein described, and
(b) a first and a second polypeptide that are linked to each other by a disulfide bond,
wherein the immune activating Fc domain binding molecule is characterized in that the first polypeptide comprises two ectodomains of 4-1BBL or a fragment thereof that are connected to each other by a peptide linker and in that the second polypeptide comprises one ectodomain of 4-1BBL or a fragment thereof, and
(c) a half-life extending Fc domain composed of a first and a second subunit capable of stable association as herein described,
wherein the Fc domain binding moiety does not specifically bind to the half-life extending Fc domain.

In one aspect, the invention provides an immune activating fragment crystallizable (Fc) domain binding molecule comprising
(a) two Fc domain binding moiety which specifically binds to a target Fc domain as herein described, and
(b) a first and a second polypeptide that are linked to each other by a disulfide bond,
wherein the antigen binding molecule is characterized in that the first polypeptide comprises two ectodomains of 4-1BBL or a fragment thereof that are connected to each other by a peptide linker and in that the second polypeptide comprises one ectodomain of 4-1BBL or a fragment thereof, and
(c) a half-life extending Fc domain composed of a first and a second subunit capable of stable association as herein described,
wherein the Fc domain binding moiety does not specifically bind to the half-life extending Fc domain.

In a further aspect, the invention provides an immune activating fragment crystallizable (Fc) domain binding molecule as defined herein before, wherein the Fc domain binding moiety which specifically binds to a target Fc domain is selected from the group consisting of an antibody, an antibody fragment and a scaffold antigen binding protein.

In one aspect, provided is an immune activating fragment crystallizable (Fc) domain binding molecule as described herein before, wherein the Fc domain binding moiety which specifically binds to a target Fc domain is selected from the group consisting of an antibody fragment, a Fab molecule, a crossover Fab molecule, a single chain Fab molecule, a Fv molecule, a scFv molecule, a single domain antibody, or a VH and a scaffold antigen binding protein.

In one aspect, the Fc domain binding moiety which specifically binds to a target Fc domain is an a VH or a scaffold antigen binding protein.

In a particular aspect, provided is an immune activating fragment crystallizable (Fc) domain binding molecule, the Fc domain binding moiety which specifically binds to a target Fc domain is a Fab molecule or a crossover Fab molecule. In particular, the the Fc domain binding moiety which specifically binds to a target Fc domain is a Fab.

In a further aspect, provided is an immune activating fragment crystallizable (Fc) domain binding molecule according to the invention, wherein a peptide comprising two ectodomains of 4-1BBL or a fragment thereof connected to each other by a first peptide linker is fused at its C-terminus to the CH1 domain of a heavy chain by a second peptide linker and wherein one ectodomain of said 4-1BBL or a fragment thereof is fused at the its C-terminus to the CL domain on a light chain by a third peptide linker.

In another aspect, provided is an immune activating fragment crystallizable (Fc) domain binding molecule according to the invention, wherein a peptide comprising two ectodomains of 4-1BBL or a fragment thereof connected to each other by a first peptide linker is fused at its C-terminus to the CL domain of a heavy chain by a second peptide linker and wherein one ectodomain of said 4-1BBL or a fragment thereof is fused at the its C-terminus to the CH1 domain on a light chain by a third peptide linker.

In a further aspect, the invention is concerned with an immune activating fragment crystallizable (Fc) domain binding molecule according to the invention, wherein a peptide comprising two ectodomains of a 4-1BBL or a fragment thereof connected to each other by a first peptide linker is fused at its C-terminus to the CL domain of a light chain by a second peptide linker and wherein one ectodomain of said 4-1BBL or a fragment thereof is fused at the its C-terminus to the CH1 domain of the heavy chain by a third peptide linker.

In a particular aspect, the invention relates to an immune activating fragment crystallizable (Fc) domain binding molecule as defined above, wherein the peptide linker is (G4S)2.

In another aspect, the an immune activating fragment crystallizable (Fc) domain binding molecule as defined herein before comprises an Fc domain composed of a first and a second subunit capable of stable association.

In one embodiment, provided is an immune activating fragment crystallizable (Fc) domain binding molecule comprising:
(a) a first light chain comprising an amino acid sequence of SEQ ID NO:10,
(b) a second light chain comprising an amino acid sequence of SEQ ID NO:129.
(c) a first heavy chain comprising the amino acid sequence of SEQ ID NO:130; and
(d) a second heavy chain comprising the amino acid sequence of SEQ ID NO:131.

In one embodiment, provided is an immune activating fragment crystallizable (Fc) domain binding molecule comprising:
(a) a first light chain comprising an amino acid sequence of SEQ ID NO:15,
(b) a second light chain comprising an amino acid sequence of SEQ ID NO:129.
(c) a first heavy chain comprising the amino acid sequence of SEQ ID NO:130; and
(d) a second heavy chain comprising the amino acid sequence of SEQ ID NO:132.

In a further embodiment, provided is any one of the immune activating fragment crystallizable (Fc) domain binding molecule as described herein above further comprising a substitution at position P329 (numbering according to Kabat EU index) by an amino acid selected from the list consisting of arginine (R), leucine (L), isoleucine (I), and alanine (A).

Immune Activating Fc Domain Binding Molecules Comprising an Fc Receptor Immune Activating Moiety

In one aspect of the present invention, the immune activating moiety is an Fc receptor. In one embodiment, the Fc receptor is an activating Fc receptor. In one embodiment, the immune activating moiety induced ACDD. In one embodiment, the Fc receptor is selected from the list consisting of FcγRIIIa (CD16a), FcγRI (CD64), FcγRIIa (CD32), and FcαRI (CD89). In a particular embodiment, the immune activating moiety is FcγRIIIa (CD16a), or a fragment thereof. In a particular embodiment, the immune activating moiety is FcγRIIa (CD32), or a fragment thereof. In a particular embodiment, the immune activating moiety is FcαRI (CD89), or a fragment thereof.

Further specific immune activating Fc domain binding molecules according to the invention Provided is an immune activating Fc domain binding molecule comprising
(a) a Fab molecule comprising:
   (i) a heavy chain variable region (VH) comprising
      (a) the heavy chain complementarity-determining region (CDR H) 1 amino acid sequence RYWMN (SEQ ID NO:1);
      (b) a CDR H2 amino acid sequence selected from the group consisting of EITPDSSTINYTPSLKD (SEQ ID NO:2), EITPDSSTINYTPSLKG (SEQ ID NO:11) and EITPDSSTINYAPSLKG (SEQ ID NO:16); and
      (c) the CDR H3 amino acid sequence PYDYGAWFAS (SEQ ID NO:3); and
   (ii) a light chain variable region (VL) comprising
      (d) the light chain complementary-determining region (CDR L) 1 amino acid sequence RSSTGAVTTSNYAN (SEQ ID NO:4);
      (e) the CDR L2 amino acid sequence GTNKRAP (SEQ ID NO:5); and
      (f) the CDR L3 amino acid sequence ALWYSNHWV (SEQ ID NO:6).
   (b) an IgG1 Fc domain comprising a substitution at position P329 (numbering according to Kabat EU index) by arginine (R), and
   (c) an immune activating moiety as herein before described.

Provided is an immune activating Fc domain binding molecule comprising
(a) a Fab molecule comprising:
   (i) a heavy chain variable region (VH) comprising
      (a) the heavy chain complementarity-determining region (CDR H) 1 amino acid sequence RYWMN (SEQ ID NO:1);
      (b) the CDR H2 amino acid sequence EITPDSSTINYTPSLKD (SEQ ID NO:2); and
      (c) the CDR H3 amino acid sequence PYDYGAWFAS (SEQ ID NO:3); and
   (ii) a light chain variable region (VL) comprising
      (d) the light chain complementary-determining region (CDR L) 1 amino acid sequence RSSTGAVTTSNYAN (SEQ ID NO:4);
      (e) the CDR L2 amino acid sequence GTNKRAP (SEQ ID NO:5); and
      (f) the CDR L3 amino acid sequence ALWYSNHWV (SEQ ID NO:6).
(b) an IgG1 Fc domain comprising a substitution at position P329 (numbering according to Kabat EU index) by arginine (R), and
(c) an immune activating moiety as herein before described.

Provided is an immune activating Fc domain binding molecule comprising
(a) a Fab molecule comprising:
   (i) a heavy chain variable region (VH) comprising
      (a) the heavy chain complementarity-determining region (CDR H) 1 amino acid sequence RYWMN (SEQ ID NO:1);
      (b) the CDR H2 amino acid sequence EITPDSSTINYTPSLKG (SEQ ID NO:11); and
      (c) the CDR H3 amino acid sequence PYDYGAWFAS (SEQ ID NO:3); and
   (ii) a light chain variable region (VL) comprising
      (d) the light chain complementary-determining region (CDR L) 1 amino acid sequence RSSTGAVTTSNYAN (SEQ ID NO:4);
      (e) the CDR L2 amino acid sequence GTNKRAP (SEQ ID NO:5); and
      (f) the CDR L3 amino acid sequence ALWYSNHWV (SEQ ID NO:6).
(b) an IgG1 Fc domain comprising a substitution at position P329 (numbering according to Kabat EU index) by arginine (R), and
(c) an immune activating moiety as herein before described.

Provided is an immune activating Fc domain binding molecule comprising
(a) a Fab molecule comprising:
   (i) a heavy chain variable region (VH) comprising
      (a) the heavy chain complementarity-determining region (CDR H) 1 amino acid sequence RYWMN (SEQ ID NO:1);
      (b) the CDR H2 amino acid sequence EITPDSSTINYAPSLKG (SEQ ID NO:16); and
      (c) the CDR H3 amino acid sequence PYDYGAWFAS (SEQ ID NO:3); and
   (ii) a light chain variable region (VL) comprising
      (d) the light chain complementary-determining region (CDR L) 1 amino acid sequence RSSTGAVTTSNYAN (SEQ ID NO:4);
      (e) the CDR L2 amino acid sequence GTNKRAP (SEQ ID NO:5); and
      (f) the CDR L3 amino acid sequence ALWYSNHWV (SEQ ID NO:6).
(b) an IgG1 Fc domain comprising a substitution at position P329 (numbering according to Kabat EU index) by arginine (R), and
(c) an immune activating moiety as herein before described.

Targeting Antibody

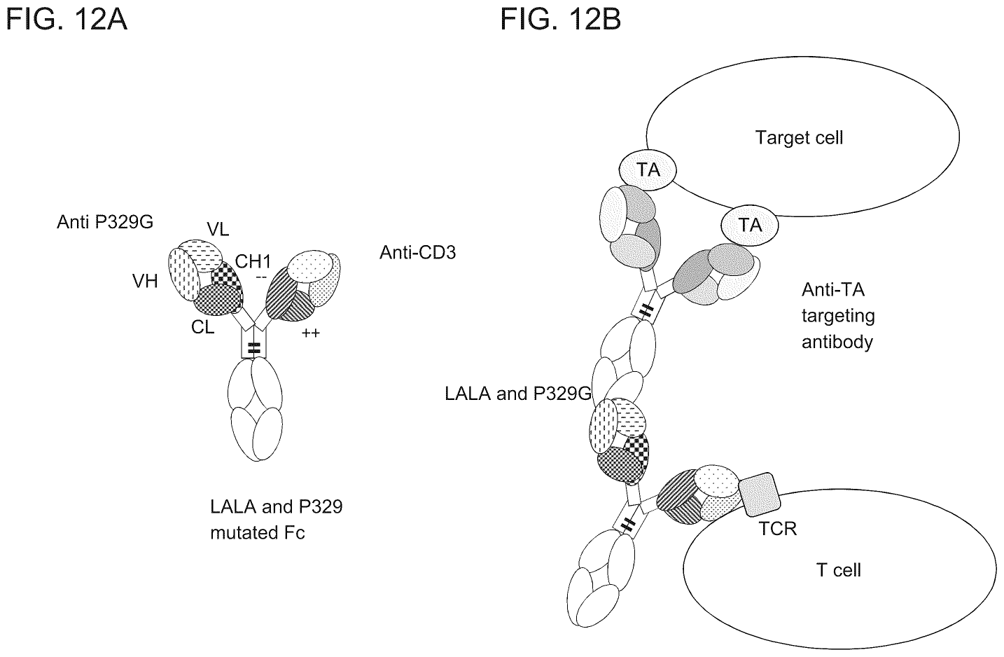

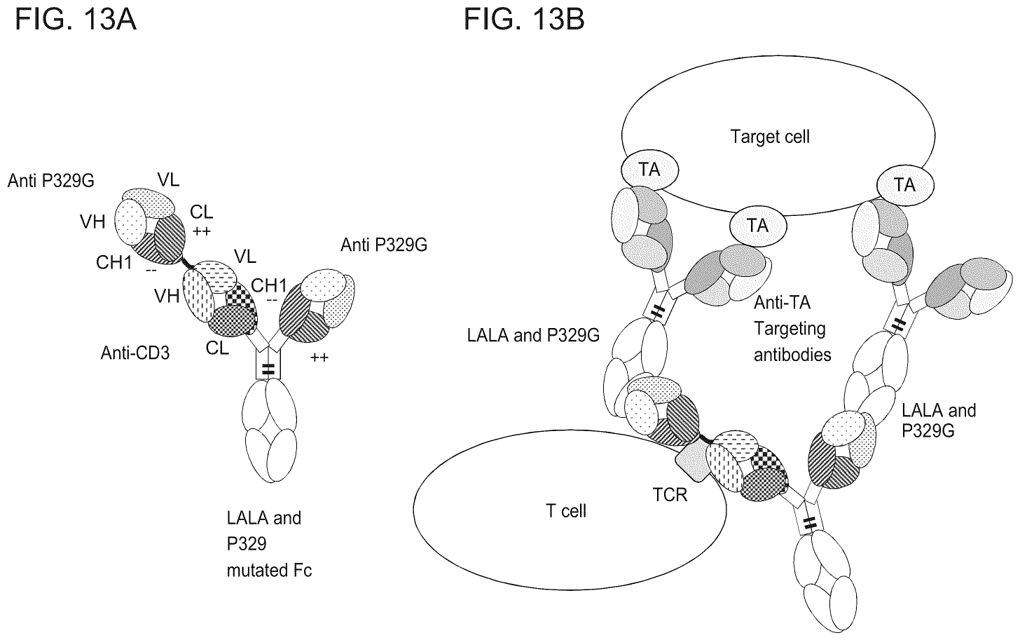

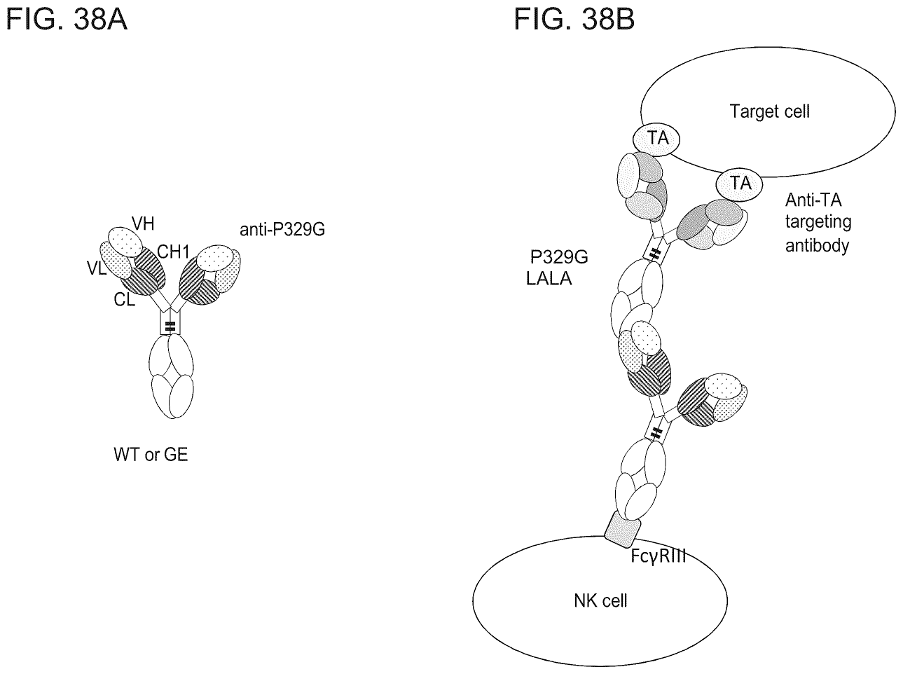

The targeting antibody is capable of binding to the target cell (as illustrated in 43). As herein before described, the targeting antibody comprises the target Fc domain comprising the first set of at least one amino acid substitution. The targeting antibody may comprise any of the modifications and/or substitutions hereinabove described, in particular the first set of at least one amino acid substitution as herein above described. According to the concept of the present invention and as herein before described, the targeting antibody bridge/link/connect the immune activating Fc domain binding molecules of the present invention and the target cell (see e.g FIG. 1, FIG. 12, FIG. 13, FIG. 38, and FIG. 43).

In one aspect, the invention provides targeting antibodies that bind to a target antigen on a target cell. In one aspect, provided are isolated targeting antibodies that bind to a target antigen on a target cell. In one aspect, the invention provides antibodies that specifically bind to an antigen selected from the list consisting of PD-L1, CD20, FolR1, CD25, FAP, EpCAM, STEAP1, Her2 and CEA.

In another aspect, the targeting antibody is capable of binding to an immune cell, in particular a T cell. In a preferred embodiment, the targeting antibody is capable of binding to PD-1. Targeting PD-1 is particularly useful to target (deliver) cytokines to T cells. In one embodiment, the targeting antibody is capable of binding to PD-1.

In a further aspect, the targeting antibody as described herein is of IgG1 isotype/subclass.

In a further aspect, the targeting antibody as described herein comprises the heavy chain of SEQ ID NO:146 or the constant parts thereof. In another aspect, the antibody according to any of the above aspects comprises a light chain of SEQ ID:147 or the constant parts thereof.

In a further aspect, the targeting antibody as described herein comprises the heavy chain of SEQ ID NO:148 or the constant parts thereof. In another aspect, the antibody according to any of the above aspects comprises a light chain of SEQ ID: 149 or the constant parts thereof.

In a further aspect, the targeting antibody as described herein comprises the heavy chain of SEQ ID NO:150 or the constant parts thereof. In another aspect, the antibody according to any of the above aspects comprises a light chain of SEQ ID: 151 or the constant parts thereof.

In a further aspect, the targeting antibody as described herein comprises the heavy chain of SEQ ID NO: 152 or the constant parts thereof. In another aspect, the antibody according to any of the above aspects comprises a light chain of SEQ ID: 153 or the constant parts thereof.

In a further aspect, the targeting antibody as described herein comprises the heavy chain of SEQ ID NO: 154 or the constant parts thereof. In another aspect, the antibody according to any of the above aspects comprises a light chain of SEQ ID: 155 or the constant parts thereof.

In a further aspect, the targeting antibody as described herein comprises the heavy chain of SEQ ID NO: 156 or the constant parts thereof. In another aspect, the antibody according to any of the above aspects comprises a light chain of SEQ ID: 157 or the constant parts thereof.

In a further aspect, the targeting antibody as described herein comprises the heavy chain of SEQ ID NO: 158 or the constant parts thereof. In another aspect, the antibody according to any of the above aspects comprises a light chain of SEQ ID: 159 or the constant parts thereof.

In a further aspect, the targeting antibody as described herein comprises the heavy chain of SEQ ID NO: 160 or the constant parts thereof. In another aspect, the antibody according to any of the above aspects comprises a light chain of SEQ ID: 161 or the constant parts thereof.

In a further aspect, the targeting antibody as described herein comprises the heavy chain of SEQ ID NO: 162 or the constant parts thereof. In another aspect, the antibody according to any of the above aspects comprises a light chain of SEQ ID: 163 or the constant parts thereof.

In a further aspect, the targeting antibody as described herein comprises the heavy chain of SEQ ID NO: 164 or the constant parts thereof. In another aspect, the antibody according to any of the above aspects comprises a light chain of SEQ ID: 165 or the constant parts thereof.

In one aspect, additionally the C-terminal glycine (Gly446) is present in the heavy chain sequences hereinabove described. In one aspect, additionally the C-terminal glycine (Gly446) and the C-terminal lysine (Lys447) is present.

Polynucleotides

The invention further provides isolated polynucleotides encoding an immune activating Fc domain binding molecule as described herein or a fragment thereof. In some embodiments, said fragment is an antigen binding fragment.

The polynucleotides encoding immune activating Fc domain binding molecules of the invention may be expressed as a single polynucleotide that encodes the entire immune activating Fc domain binding molecule or as multiple (e.g., two or more) polynucleotides that are co-expressed.

Polypeptides encoded by polynucleotides that are co-expressed may associate through, e.g., disulfide bonds or other means to form a functional immune activating Fc domain binding molecule. For example, the light chain portion of a Fab molecule may be encoded by a separate polynucleotide from the portion of the immune activating Fc domain binding molecule comprising the heavy chain portion of the Fab molecule, an Fc domain subunit and optionally (part of) another Fab molecule. When co-expressed, the heavy chain polypeptides will associate with the light chain polypeptides to form the Fab molecule. In another example, the portion of the immune activating Fc domain binding molecule comprising one of the two Fc domain subunits and optionally (part of) one or more Fab molecules could be encoded by a separate polynucleotide from the portion of the immune activating Fc domain binding molecule comprising the the other of the two Fc domain subunits and optionally (part of) a Fab molecule. When co-expressed, the Fc domain subunits will associate to form the Fc domain.

In some embodiments, the isolated polynucleotide encodes the entire immune activating Fc domain binding molecule according to the invention as described herein. In other embodiments, the isolated polynucleotide encodes a polypeptides comprised in the immune activating Fc domain binding molecule according to the invention as described herein.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

Recombinant Methods

Immune activating Fc domain binding molecules of the invention may be obtained, for example, by solid-state peptide synthesis (e.g. Merrifield solid phase synthesis) or recombinant production. For recombinant production one or more polynucleotide encoding the immune activating Fc domain binding molecule (fragment), e.g., as described above, is isolated and inserted into one or more vectors for further cloning and/or expression in a host cell. Such polynucleotide may be readily isolated and sequenced using conventional procedures. In one embodiment a vector, preferably an expression vector, comprising one or more of the polynucleotides of the invention is provided. Methods which are well known to those skilled in the art can be used to construct expression vectors containing the coding sequence of a immune activating Fc domain binding molecule (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989). The expression vector can be part of a plasmid, virus, or may be a nucleic acid fragment. The expression vector includes an expression cassette into which the polynucleotide encoding the immune activating Fc domain binding molecule (fragment) (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the immune activating Fc domain binding molecule (fragment) of the invention, or variant or derivative thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus (e.g. the early promoter), and retroviruses (such as, e.g. Rous sarcoma virus). Other transcription control regions include those derived from vertebrate genes such as actin, heat shock protein, bovine growth hormone and rabbit α-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the immune activating Fc domain binding molecule is desired, DNA encoding a signal sequence may be placed upstream of the nucleic acid encoding a immune activating Fc domain binding molecule of the invention or a fragment thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the immune activating Fc domain binding molecule may be included within or at the ends of the immune activating Fc domain binding molecule (fragment) encoding polynucleotide.

In a further embodiment, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one such embodiment a host cell comprises (e.g. has been transformed or transfected with) a vector comprising a polynucleotide that encodes (part of) an immune activating Fc domain binding molecule of the invention. As used herein, the term “host cell” refers to any kind of cellular system which can be engineered to generate the immune activating Fc domain binding molecules of the invention or fragments thereof. Host cells suitable for replicating and for supporting expression of immune activating Fc domain binding molecules are well known in the art. Such cells may be transfected or transduced as appropriate with the particular expression vector and large quantities of vector containing cells can be grown for seeding large scale fermenters to obtain sufficient quantities of the immune activating Fc domain binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B.K.C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NS0, Sp20 cell).

Standard technologies are known in the art to express foreign genes in these systems. Cells expressing a polypeptide comprising either the heavy or the light chain of an antigen binding domain such as an antibody, may be engineered so as to also express the other of the antibody chains such that the expressed product is an antibody that has both a heavy and a light chain.

In one embodiment, a method of producing a immune activating Fc domain binding molecule according to the invention is provided, wherein the method comprises culturing a host cell comprising a polynucleotide encoding the immune activating Fc domain binding molecule, as provided herein, under conditions suitable for expression of the immune activating Fc domain binding molecule, and recovering the immune activating Fc domain binding molecule from the host cell (or host cell culture medium).

The components of the immune activating Fc domain binding molecule of the invention are genetically fused to each other. Immune activating Fc domain binding molecule can be designed such that its components are fused directly to each other or indirectly through a linker sequence. The composition and length of the linker may be determined in accordance with methods well known in the art and may be tested for efficacy. Examples of linker sequences between different components of immune activating Fc domain binding molecules of the invention are found in the sequences provided herein. Additional sequences may also be included to incorporate a cleavage site to separate the individual components of the fusion if desired, for example an endopeptidase recognition sequence.

In certain embodiments the one or more antigen binding moieties of the immune activating Fc domain binding molecules of the invention comprise at least an antibody variable region capable of binding an antigenic determinant. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty).

Any animal species of antibody, antibody fragment, antigen binding domain or variable region can be used in the immune activating Fc domain binding molecules of the invention. Non-limiting antibodies, antibody fragments, antigen binding domains or variable regions useful in the present invention can be of murine, primate, or human origin. If the immune activating Fc domain binding molecule is intended for human use, a chimeric form of antibody may be used wherein the constant regions of the antibody are from a human. A humanized or fully human form of the antibody can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, N J, 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain embodiments, the antigen binding moieties useful in the present invention are engineered to have enhanced binding affinity according to, for example, the methods disclosed in U.S. Pat. Appl. Publ. No. 2004/0132066, the entire contents of which are hereby incorporated by reference. The ability of the immune activating Fc domain binding molecule of the invention to bind to a specific antigenic determinant can be measured either through an enzyme-linked immunosorbent assay (ELISA) or other techniques familiar to one of skill in the art, e.g. surface plasmon resonance technique (analyzed on a BIACORE T100 system) (Liljeblad, et al., Glyco J 17, 323-329 (2000)), and traditional binding assays (Heeley, Endocr Res 28, 217-229 (2002)). Competition assays may be used to identify an antibody, antibody fragment, antigen binding domain or variable domain that competes with a reference antibody for binding to a particular antigen, e.g. an antibody that competes with the V9 antibody for binding to CD3. In certain embodiments, such a competing antibody binds to the same epitope (e.g. a linear or a conformational epitope) that is bound by the reference antibody. Detailed exemplary methods for mapping an epitope to which an antibody binds are provided in Morris (1996) “Epitope Mapping Protocols,” in Methods in Molecular Biology vol. 66 (Humana Press, Totowa, NJ). In an exemplary competition assay, immobilized antigen (e.g. CD3) is incubated in a solution comprising a first labeled antibody that binds to the antigen (e.g. V9 antibody, described in U.S. Pat. No. 6,054,297) and a second unlabeled antibody that is being tested for its ability to compete with the first antibody for binding to the antigen. The second antibody may be present in a hybridoma supernatant. As a control, immobilized antigen is incubated in a solution comprising the first labeled antibody but not the second unlabeled antibody. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to the antigen. See Harlow and Lane (1988) Antibodies: A Laboratory Manual ch.14 (Cold Spring Harbor Laboratory, Cold Spring Harbor, NY).

Immune activating Fc domain binding molecules prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the immune activating Fc domain binding molecule binds. For example, for affinity chromatography purification of immune activating Fc domain binding molecules of the invention, a matrix with protein A or protein G may be used. Sequential Protein A or G affinity chromatography and size exclusion chromatography can be used to isolate an immune activating Fc domain binding molecule essentially as described in the Examples. The purity of the immune activating Fc domain binding molecule can be determined by any of a variety of well known analytical methods including gel electrophoresis, high pressure liquid chromatography, and the like.

Assays

Immune activating Fc domain binding molecules provided herein may be identified, screened for, or characterized for their physical/chemical properties and/or biological activities by various assays known in the art.

Affinity Assays

The affinity of the immune activating Fc domain binding molecule for an Fc receptor or a target antigen can be determined in accordance with the methods set forth in the Examples by surface plasmon resonance (SPR), using standard instrumentation such as a BIAcore instrument (GE Healthcare), and receptors or target proteins such as may be obtained by recombinant expression. Alternatively, binding of immune activating Fc domain binding molecules for different receptors or target antigens may be evaluated using cell lines expressing the particular receptor or target antigen, for example by flow cytometry (FACS). A specific illustrative and exemplary embodiment for measuring binding affinity is described in the following and in the Examples below.

According to one embodiment, K_{D} is measured by surface plasmon resonance using a BIACORE® T100 machine (GE Healthcare) at 25° C.

To analyze the interaction between the Fc-portion and Fc receptors, His-tagged recombinant Fc-receptor is captured by an anti-Penta His antibody (Qiagen) immobilized on CM5 chips and the bispecific constructs are used as analytes. Briefly, carboxymethylated dextran biosensor chips (CM5, GE Healthcare) are activated with N-ethyl-N′-(3-dimethylaminopropyl)-carbodiimide hydrochloride (EDC) and N-hydroxysuccinimide (NHS) according to the supplier's instructions. Anti Penta-His antibody is diluted with 10 mM sodium acetate, pH 5.0, to 40 pg/ml before injection at a flow rate of 5 μl/min to achieve approximately 6500 response units (RU) of coupled protein. Following the injection of the ligand, 1 M ethanolamine is injected to block unreacted groups. Subsequently the Fc-receptor is captured for 60 s at 4 or 10 nM. For kinetic measurements, four-fold serial dilutions of the bispecific construct (range between 500 nM and 4000 nM) are injected in HBS-EP (GE Healthcare, 10 mM HEPES, 150 mM NaCl, 3 mM EDTA, 0.05% Surfactant P20, pH 7.4) at 25° C. at a flow rate of 30 μl/min for 120 s.

To determine the affinity to the target antigen, bispecific constructs are captured by an anti human Fab specific antibody (GE Healthcare) that is immobilized on an activated CM5-sensor chip surface as described for the anti Penta-His antibody. The final amount of coupled protein is is approximately 12000 R U. The bispecific constructs are captured for 90 s at 300 nM. The target antigens are passed through the flow cells for 180 s at a concentration range from 250 to 1000 nM with a flowrate of 30 μl/min. The dissociation is monitored for 180 s.

Bulk refractive index differences are corrected for by subtracting the response obtained on reference flow cell. The steady state response was used to derive the dissociation constant K_{D} by non-linear curve fitting of the Langmuir binding isotherm. Association rates (kₒₙ) and dissociation rates (k_{off}) are calculated using a simple one-to-one Langmuir binding model (BIACORE® T100 Evaluation Software version 1.1.1) by simultaneously fitting the association and dissociation sensorgrams. The equilibrium dissociation constant (K_{D}) is calculated as the ratio k_{off}/kₒₙ. See, e.g., Chen et al., J Mol Biol 293, 865-881 (1999).

Activity Assays

Biological activity of the immune activating Fc domain binding molecules of the invention can be measured by various assays as described in the Examples. Biological activities may for example include the induction of proliferation of T cells, the induction of signaling in T cells, the induction of expression of activation markers in T cells, the induction of cytokine secretion by T cells, the induction of lysis of target cells such as tumor cells, and the induction of tumor regression and/or the improvement of survival.

Compositions, Formulations, and Routes of Administration

In a further aspect, the invention provides pharmaceutical compositions comprising any of the immune activating Fc domain binding molecules provided herein, e.g., for use in any of the below therapeutic methods. In one embodiment, a pharmaceutical composition comprises any of the immune activating Fc domain binding molecules provided herein and a pharmaceutically acceptable carrier. In another embodiment, a pharmaceutical composition comprises any of the immune activating Fc domain binding molecules provided herein and at least one additional therapeutic agent, e.g., as described below.

Further provided is a method of producing a immune activating Fc domain binding molecule of the invention in a form suitable for administration in vivo, the method comprising (a) obtaining a immune activating Fc domain binding molecule according to the invention, and (b) formulating the molecule with at least one pharmaceutically acceptable carrier, whereby a preparation of the molecule is formulated for administration in vivo.

Pharmaceutical compositions of the present invention comprise a therapeutically effective amount of one or more immune activating Fc domain binding molecule dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one immune activating Fc domain binding molecule and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated. The composition may comprise different types of carriers depending on whether it is to be administered in solid, liquid or aerosol form, and whether it need to be sterile for such routes of administration as injection. immune activating Fc domain binding molecules of the present invention (and any additional therapeutic agent) can be administered intravenously, intradermally, intraarterially, intraperitoneally, intralesionally, intracranially, intraarticularly, intraprostatically, intrasplenically, intrarenally, intrapleurally, intratracheally, intranasally, intravitreally, intravaginally, intrarectally, intratumorally, intramuscularly, intraperitoneally, subcutaneously, subconjunctivally, intravesicularlly, mucosally, intrapericardially, intraumbilically, intraocularally, orally, topically, locally, by inhalation (e.g. aerosol inhalation), injection, infusion, continuous infusion, localized perfusion bathing target cells directly, via a catheter, via a lavage, in cremes, in lipid compositions (e.g. liposomes), or by other method or any combination of the forgoing as would be known to one of ordinary skill in the art (see, for example, Remington's Pharmaceutical Sciences, 18th Ed. Mack Printing Company, 1990, incorporated herein by reference). Parenteral administration, in particular intravenous injection, is most commonly used for administering the immune activating Fc domain binding molecules of the invention.

Parenteral compositions include those designed for administration by injection, e.g. subcutaneous, intradermal, intralesional, intravenous, intraarterial intramuscular, intrathecal or intraperitoneal injection. For injection, the immune activating Fc domain binding molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the immune activating Fc domain binding molecules may be in powder form for constitution with a suitable vehicle, e.g., sterile pyrogen-free water, before use. Sterile injectable solutions are prepared by incorporating the immune activating Fc domain binding molecules of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

In addition to the compositions described previously, the immune activating Fc domain binding molecules may also be formulated as a depot preparation. Such long acting formulations may be administered by implantation (for example subcutaneously or intramuscularly) or by intramuscular injection. Thus, for example, the immune activating Fc domain binding molecules may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the immune activating Fc domain binding molecules of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen. The immune activating Fc domain binding molecules may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

Therapeutic Methods and Compositions

Any of the immune activating Fc domain binding molecules provided herein may be used in therapeutic methods. Molecules of the invention can be used as immunotherapeutic agents, for example in the treatment of cancers.

For use in therapeutic methods, immune activating Fc domain binding molecules of the invention would be formulated, dosed, and administered in a fashion consistent with good medical practice.

Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, immune activating Fc domain binding molecules of the invention for use as a medicament are provided. In further aspects, immune activating Fc domain binding molecules of the invention for use in treating a disease are provided. In certain embodiments, immune activating Fc domain binding molecules of the invention for use in a method of treatment are provided. In one embodiment, the invention provides an immune activating Fc domain binding molecule as described herein for use in the treatment of a disease in an individual in need thereof. In certain embodiments, the invention provides an immune activating Fc domain binding molecule for use in a method of treating an individual having a disease comprising administering to the individual a therapeutically effective amount of the immune activating Fc domain binding molecule. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. In further embodiments, the invention provides a immune activating Fc domain binding molecule as described herein for use in inducing lysis of a target cell, particularly a tumor cell. In certain embodiments, the invention provides a immune activating Fc domain binding molecule for use in a method of inducing lysis of a target cell, particularly a tumor cell, in an individual comprising administering to the individual an effective amount of the immune activating Fc domain binding molecule to induce lysis of a target cell. An “individual” according to any of the above embodiments is a mammal, preferably a human.

In a further aspect, the invention provides for the use of an immune activating Fc domain binding molecule of the invention in the manufacture or preparation of a medicament. In one embodiment the medicament is for the treatment of a disease in an individual in need thereof. In a further embodiment, the medicament is for use in a method of treating a disease comprising administering to an individual having the disease a therapeutically effective amount of the medicament. In certain embodiments the disease to be treated is a proliferative disorder. In a particular embodiment the disease is cancer. In one embodiment, the method further comprises administering to the individual a therapeutically effective amount of at least one additional therapeutic agent, e.g., an anti-cancer agent if the disease to be treated is cancer. In a further embodiment, the medicament is for inducing lysis of a target cell, particularly a tumor cell. In still a further embodiment, the medicament is for use in a method of inducing lysis of a target cell, particularly a tumor cell, in an individual comprising administering to the individual an effective amount of the medicament to induce lysis of a target cell. An “individual” according to any of the above embodiments may be a mammal, preferably a human.

In a further aspect, the invention provides a method for treating a disease. In one embodiment, the method comprises administering to an individual having such disease a therapeutically effective amount of an immune activating Fc domain binding molecule of the invention. In one embodiment a composition is administered to said individual, comprising the immune activating Fc domain binding molecule of the invention in a pharmaceutically acceptable form. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. An “individual” according to any of the above embodiments may be a mammal, preferably a human.

In a further aspect, the invention provides a method for inducing lysis of a target cell, particularly a tumor cell. In one embodiment the method comprises contacting a target cell with a immune activating Fc domain binding molecule of the invention in the presence of a T cell, particularly a cytotoxic T cell. In a further aspect, a method for inducing lysis of a target cell, particularly a tumor cell, in an individual is provided. In one such embodiment, the method comprises administering to the individual an effective amount of an immune activating Fc domain binding molecule to induce lysis of a target cell. In one embodiment, an “individual” is a human.

In certain embodiments the disease to be treated is a proliferative disorder, particularly cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using a immune activating Fc domain binding molecule of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan readily recognizes that in many cases the immune activating Fc domain binding molecule may not provide a cure but may only provide partial benefit. In some embodiments, a physiological change having some benefit is also considered therapeutically beneficial. Thus, in some embodiments, an amount of immune activating Fc domain binding molecule that provides a physiological change is considered an “effective amount” or a “therapeutically effective amount”. The subject, patient, or individual in need of treatment is typically a mammal, more specifically a human.

In some embodiments, an effective amount of a immune activating Fc domain binding molecule of the invention is administered to a cell. In other embodiments, a therapeutically effective amount of an immune activating Fc domain binding molecule of the invention is administered to an individual for the treatment of disease.

For the prevention or treatment of disease, the appropriate dosage of an immune activating Fc domain binding molecule of the invention (when used alone or in combination with one or more other additional therapeutic agents) will depend on the type of disease to be treated, the route of administration, the body weight of the patient, the type molecule, the severity and course of the disease, whether the molecule is administered for preventive or therapeutic purposes, previous or concurrent therapeutic interventions, the patient's clinical history and response to the molecule, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The immune activating Fc domain binding molecule is suitably administered to the patient at one time or over a series of treatments. Depending on the type and severity of the disease, about 1 pg/kg to 15 mg/kg (e.g. 0.1 mg/kg-10 mg/kg) of immune activating Fc domain binding molecule can be an initial candidate dosage for administration to the patient, whether, for example, by one or more separate administrations, or by continuous infusion. One typical daily dosage might range from about 1 pg/kg to 100 mg/kg or more, depending on the factors mentioned above. For repeated administrations over several days or longer, depending on the condition, the treatment would generally be sustained until a desired suppression of disease symptoms occurs. One exemplary dosage of the immune activating Fc domain binding molecule would be in the range from about 0.005 mg/kg to about 10 mg/kg. In other non-limiting examples, a dose may also comprise from about 1 microgram/kg body weight, about 5 microgram/kg body weight, about 10 microgram/kg body weight, about 50 microgram/kg body weight, about 100 microgram/kg body weight, about 200 microgram/kg body weight, about 350 microgram/kg body weight, about 500 microgram/kg body weight, about 1 milligram/kg body weight, about 5 milligram/kg body weight, about 10 milligram/kg body weight, about 50 milligram/kg body weight, about 100 milligram/kg body weight, about 200 milligram/kg body weight, about 350 milligram/kg body weight, about 500 milligram/kg body weight, to about 1000 mg/kg body weight or more per administration, and any range derivable therein. In non-limiting examples of a derivable range from the numbers listed herein, a range of about 5 mg/kg body weight to about 100 mg/kg body weight, about 5 microgram/kg body weight to about 500 milligram/kg body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the immune activating Fc domain binding molecule). An initial higher loading dose, followed by one or more lower doses may be administered. However, other dosage regimens may be useful. The progress of this therapy is easily monitored by conventional techniques and assays.

The immune activating Fc domain binding molecules of the invention will generally be used in an amount effective to achieve the intended purpose. For use to treat or prevent a disease condition, the immune activating Fc domain binding molecules of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the immune activating Fc domain binding molecules which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the immune activating Fc domain binding molecules may not be related to plasma concentration. One having skill in the art will be able to optimize therapeutically effective local dosages without undue experimentation.

A therapeutically effective dose of the immune activating Fc domain binding molecules described herein will generally provide therapeutic benefit without causing substantial toxicity. Toxicity and therapeutic efficacy of an immune activating Fc domain binding molecule can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Immune activating Fc domain binding molecules that exhibit large therapeutic indices are preferred. In one embodiment, the immune activating Fc domain binding molecule according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with immune activating Fc domain binding molecules of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

Other Agents and Treatments

The immune activating Fc domain binding molecules of the invention may be administered in combination with one or more other agents in therapy. For instance, an immune activating Fc domain binding molecule of the invention may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent. Such other agents are suitably present in combination in amounts that are effective for the purpose intended. The effective amount of such other agents depends on the amount of immune activating Fc domain binding molecule used, the type of disorder or treatment, and other factors discussed above. The immune activating Fc domain binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the immune activating Fc domain binding molecule of the invention can occur prior to, simultaneously, and/or following, administration of the additional therapeutic agent and/or adjuvant. Immune activating Fc domain binding molecules of the invention can also be used in combination with radiation therapy.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a immune activating Fc domain binding molecule of the invention. The label or package insert indicates that the composition is used for treating the condition of choice. Moreover, the article of manufacture may comprise (a) a first container with a composition contained therein, wherein the composition comprises a immune activating Fc domain binding molecule of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Numbered Embodiments Describing Preferred Aspect of the Invention

1. An immune activating fragment crystallizable (Fc) domain binding molecule comprising
(a) an Fc domain binding moiety that specifically binds to a target Fc domain comprising a first set of at least one amino acid substitution, and
(b) an immune activating moiety.

2. The immune activating Fc domain binding molecule of embodiments 1, wherein the first set of at least one amino acid substitution reduce binding to an Fc receptor and/or reduce effector function.

3. The immune activating Fc domain binding molecule of embodiments 1, wherein the first set of at least one amino acid substitution increase binding to an Fc receptor and/or increase effector function.

4. The immune activating Fc domain binding molecule of any one of embodiments 1-3, further comprising
(c) a half-life extending Fc,
wherein the Fc domain binding moiety does not specifically bind to the half-life extending Fc domain.

5. The immune activating Fc domain binding molecule of any one of embodiments 1-4, wherein the half-life extending Fc domain comprises a second set of at least one amino acid substitution.

6. The immune activating Fc domain binding molecule of embodiment 5, wherein the second set of at least one amino acid substitution reduce binding to an Fc receptor and/or reduce effector function.

7. The immune activating Fc domain binding molecule of embodiment 6, wherein the second set of at least one amino acid substitution increase binding to an Fc receptor and/or increase effector function.

8. The immune activating Fc domain binding molecule of any one of embodiments 1-7, wherein the target Fc domain and/or the half-life extending Fc domain is composed of a first and a second subunit capable of stable association.

9. The immune activating Fc domain binding molecule of any one of embodiments 1-8, wherein the target Fc domain and/or the half-life extending Fc domain is a human Fc domain.

10. The immune activating Fc domain binding molecule of any one of embodiments 1-9, wherein the target Fc domain and/or the half-life extending Fc domain is an IgG Fc domain, specifically an IgG₁ or IgG₄ Fc domain.

11. The immune activating Fc domain binding molecule of embodiment 10, wherein the target Fc domain exhibits reduced binding affinity to an Fc receptor and/or reduced effector function, as compared to a native IgG₁ Fc domain.

12. The immune activating Fc domain binding molecule of embodiment 10, wherein the half-life extending Fc domain exhibits reduced binding affinity to an Fc receptor and/or reduced effector function, as compared to a native IgG₁ Fc domain.

13. The immunoactivatig molecule of any one of embodiments 1-12, wherein the target Fc domain and/or the half-life extending Fc domain is glycoengineered to increase binding to an Fc receptor and/or increase effector function

14. The immunoactivating molecule of any one of embodiments 1-13, wherein the target Fc domain and/or the half-life extending Fc domain has a reduced level of fucose residues, and/or the oligosaccharides of the target Fc domain and/or the half-life extending Fc domain are bisected.

15. The immune activating Fc domain binding molecule of any one of embodiments 1-14, wherein the first set of at least one amino acid substitution reduces binding affinity to an Fc receptor and/or effector function, and wherein the second set of at least one amino acid substitution comprises one or more amino acid substitutions at the same amino acid positions as in the first set of at least one amino acid substitution, wherein the amino acids in the second set of at least one amino acid substitution are substituted with different amino acids at the same positions compared to the first set of at least one amino acid substitution.

16. The immune activating Fc domain binding molecule of embodiment 15, wherein the second set of at least one amino acid substitution reduce binding affinity to an Fc receptor and/or effector function.

17. The immune activating Fc domain binding molecule of any one of embodiments 1-16, wherein the first set of at least one amino acid substitution comprises at least one amino acid substitution at a position selected from the list consisting of 233, 234, 235, 238, 253, 265, 269, 270, 297, 310, 331, 327, 329 and 435 (numberings according to Kabat EU index).

18. The immune activating Fc domain binding molecule of any one of embodiments 1-17, wherein the second set of at least one amino acid substitution comprises at least one amino acid substitution at a position selected from the list consisting of 233, 234, 235, 238, 253, 265, 269, 270, 297, 310, 331, 327, 329 and 435 (numberings according to Kabat EU index).

19. The immune activating Fc domain binding molecule of any one of embodiment 1-18, wherein the first set of at least one amino acid substitution comprises an amino acid substitution at position P329 (numbering according to Kabat EU index), or amino acid substitutions at positions I253, H310 and H435 (numbering according to Kabat EU index).

20. The immune activating Fc domain binding molecule of any one of embodiments 1-19, wherein the first set of at least one amino acid substitution comprises the amino acid substitution P329G (numbering according to Kabat EU index), or the amino acid substitutions I253A, H310A and H435A (numbering according to Kabat EU index).

21. The immune activating Fc domain binding molecule of any one of embodiments 1-20, wherein the first set of at least one amino acid substitution comprises the amino acid substitution P329G (numbering according to Kabat EU index) and wherein the second set of at least one amino acid substitution comprises a substitution at position P329 by an amino acid other than glycine (G) (numbering according to Kabat EU index).

22. The immune activating Fc domain binding molecule of any one of embodiments 1-21, wherein the second set of at least one amino acid substitution comprises a substitution at position P329 (numbering according to Kabat EU index) by an amino acid which is not able to form a proline sandwich between two conserved tryptophan sidechains within a Fc gamma receptor, in particular within FcgRIIIa.

23. The immune activating Fc domain binding molecule of any one of embodiments 1-22, wherein the second set of at least one amino acid substitution comprises a substitution at position P329 (numbering according to Kabat EU index) by an amino acid selected from the list consisting of arginine (R), leucine (L), isoleucine (I), and alanine (A).

24. The immune activating Fc domain binding molecule of any one of embodiment 1-23, wherein the Fc domain binding moiety is cabable of specific binding to an IgG1 Fc domain comprising the amino acid substitution P329G (numbering according to Kabat EU index) but not capable of specific binding to the parent non-mutated IgG1 Fc domain.

25. The immune activating Fc domain binding molecule of any one of embodiments 1-20, wherein the first set of at least one amino acid substitution comprises the amino acid substitution substitutions I253A, H310A and H435A (numbering according to Kabat EU index) and wherein the second set of at least one amino acid substitution comprises at least one substitution at the positions I253, H310 and H435 by an amino acid other than alanine (A) (numbering according to Kabat EU index).

26. The immune activating Fc domain binding molecule of any one of embodiments 1-20 or 25, wherein the Fc domain binding moiety is capable of specific binding to an IgG1 Fc domain comprising the amino acid mutations I253A, H310A and H435A (numbering according to Kabat EU index) but not capable of specific binding to the parent non-mutated IgG1 Fc domain.

27. The immune activating Fc domain binding molecule of any one of embodiments 1-26, wherein the first set of at least one amino acid substitution comprises at least one amino acid substitution at a position selected from the group of L234, L235 (Kabat EU index numbering).

28. The immune activating Fc domain binding molecule of any one of embodiments 1-27, wherein the second set of at least one amino acid substitution comprises at least one amino acid substitution at a position selected from the group of L234, L235 (Kabat EU index numbering).

29. The immune activating Fc domain binding molecule of any one of embodiments 1-23, wherein the target Fc domain comprises three amino acid substitutions that reduce binding to an activating Fc receptor and/or effector function wherein said amino acid substitutions are L234A, L235A and P329G (Kabat EU index numbering).

30. The immune activating Fc domain binding molecule of any one of embodiments 1-29, wherein the the half-life extending Fc domain comprises three amino acid substitutions that reduce binding to an activating Fc receptor and/or effector function wherein said amino acid substitutions are L234A, L235A and P329X (Kabat EU index numbering), wherein X is an amino acid other than glycine (G).

31. The immune activating Fc domain binding molecule of any one of embodiments 1-30, wherein the Fc receptor is an Fcγ receptor.

32. The immune activating Fc domain binding molecule of any one of embodiments 1-31, wherein the effector function is antibody-dependent cell-mediated cytotoxicity (ADCC).

33. The immune activating Fc domain binding molecule of one of embodiments 8-32, wherein the half-life extending Fc domain comprises a modification promoting the association of the first and the second subunit of the Fc domain.

34. The immune activating Fc domain binding molecule of embodiment 33, wherein in the CH3 domain of the first subunit of the half-life extending Fc domain an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the half-life extending Fc domain an amino acid residue is replaced with an amino acid residue having a smaller side chain volume, thereby generating a cavity within the CH3 domain of the second subunit within which the protuberance within the CH3 domain of the first subunit is positionable.

35. The immune activating Fc domain binding molecule of embodiment 34, wherein said amino acid residue having a larger side chain volume is selected from the group consisting of arginine (R), phenylalanine (F), tyrosine (Y), and tryptophan (W), and said amino acid residue having a smaller side chain volume is selected from the group consisting of alanine (A), serine (S), threonine (T), and valine (V).

36. The immune activating Fc domain binding molecule of embodiment 34 or 35, wherein in the CH3 domain of the first subunit of the half-life extending Fc domain the threonine residue at position 366 is replaced with a tryptophan residue (T366W), and in the CH3 domain of the second subunit of the half-life extending Fc domain the tyrosine residue at position 407 is replaced with a valine residue (Y407V), and optionally in the second subunit of the half-life extending Fc domain additionally the threonine residue at position 366 is replaced with a serine residue (T366S) and the leucine residue at position 368 is replaced with an alanine residue (L368A) (numberings according to Kabat EU index).

37. The immune activating Fc domain binding molecule of any one of embodiments 34-36, wherein in the first subunit of the half-life extending Fc domain additionally the serine residue at position 354 is replaced with a cysteine residue (S354C) or the glutamic acid residue at position 356 is replaced with a cysteine residue (E356C), and in the second subunit of the half-life extending Fc domain additionally the tyrosine residue at position 349 is replaced by a cysteine residue (Y349C) (numberings according to Kabat EU index).

38. The immune activating Fc domain binding molecule of any one of embodiments 34-37, wherein the first subunit of the half-life extending Fc domain comprises amino acid substitutions S354C and T366W, and the second subunit of the half-life extending Fc domain comprises amino acid substitutions Y349C, T366S, L368A and Y407V (numbering according to Kabat EU index).

39. The immune activating Fc domain binding molecule of any one of embodiments 1-38, wherein the Fc domain binding moiety and/or the immune activating moiety is a Fab molecule, a scFv molecule or a scFab molecule.

40. The immune activating Fc domain binding molecule of any one of embodiments 1-39, wherein the Fc domain binding moiety and/or the immune activating moiety is a Fab molecule.

41. The immune activating Fc domain binding molecule of any one of embodiments 1-24 and 27-40, wherein the Fc domain binding moiety is capable of specific binding to an IgG1 Fc domain comprising the amino acid substitution P329G (numbering according to Kabat EU index), wherein the Fc domain binding moiety comprises:
(i) a heavy chain variable region (VH) comprising
   (a) the heavy chain complementarity-determining region (CDR H) 1 amino acid sequence RYWMN (SEQ ID NO:1);
   (b) a CDR H2 amino acid sequence selected from the group consisting of EITPDSSTINYTPSLKD (SEQ ID NO:2), EITPDSSTINYTPSLKG (SEQ ID NO:11) and EITPDSSTINYAPSLKG (SEQ ID NO:16); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

42. The immune activating Fc domain binding molecule of embodiment 41, wherein the Fc domain binding moiety is capable of specific binding to an IgG1 Fc domain comprising the amino acid substitution P329G (numbering according to Kabat EU index), wherein the Fc domain binding moiety comprises:
(i) a heavy chain variable region (VH) comprising
   (a) the heavy chain complementarity-determining region (CDR H) 1 amino acid sequence RYWMN (SEQ ID NO:1);
   (b) the CDR H2 amino acid sequence EITPDSSTINYTPSLKD (SEQ ID NO:2); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

43. The immune activating Fc domain binding molecule of embodiment 41, wherein the Fc domain binding moiety is capable of specific binding to an IgG1 Fc domain comprising the amino acid substitution P329G (numbering according to Kabat EU index), wherein the Fc domain binding moiety comprises:
(i) a heavy chain variable region (VH) comprising
   (a) the heavy chain complementarity-determining region (CDR H) 1 amino acid sequence RYWMN (SEQ ID NO:1);
   (b) the CDR H2 amino acid sequence EITPDSSTINYTPSLKG (SEQ ID NO:11); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

44. The immune activating Fc domain binding molecule of embodiment 41, wherein the Fc domain binding moiety is capable of specific binding to an IgG1 Fc domain comprising the amino acid substitution P329G (numbering according to Kabat EU index), wherein the Fc domain binding moiety comprises:
(i) a heavy chain variable region (VH) comprising
   (a) the heavy chain complementarity-determining region (CDR H) 1 amino acid sequence RYWMN (SEQ ID NO:1);
   (b) the CDR H2 amino acid sequence EITPDSSTINYAPSLKG (SEQ ID NO:16); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

45. The immune activating Fc domain binding molecule of any one of embodiments 1-24 and 27-44, wherein the Fc domain binding moiety is capable of specific binding to an IgG1 Fc domain comprising the amino acid substitution P329G (numbering according to Kabat EU index), wherein the Fc domain binding moiety comprises a heavy chain variable region sequence that is at least about 95%, 96%, 97%, 98%, 99% or 100% identical to an amino acid sequence selected from the group consisting of SEQ ID NO:7, SEQ ID NO:12, SEQ ID NO:17 and SEQ ID NO:19, and a light chain variable region sequence that is at least about 95%, 96%, 97%, 98%, 99% or 100% identical to an amino acid sequence selected from the group consisting of SEQ ID NO:8 and SEQ ID NO:13.

46. The immune activating Fc domain binding molecule of embodiment 45, wherein the Fc domain binding moiety is capable of specific binding to an IgG1 Fc domain comprising the amino acid substitution P329G (numbering according to Kabat EU index), wherein the Fc domain binding moiety comprises
(i) a heavy chain variable region sequence that is at least about 95%, 96%, 97%, 98%, 99% or 100% identical to SEQ ID NO: 7 and a light chain variable region sequence that is at least about 95%, 96%, 97%, 98%, 99% or 100% identical to SEQ ID NO: 8,
(ii) a heavy chain variable region sequence that is at least about 95%, 96%, 97%, 98%, 99% or 100% identical to SEQ ID NO: 12 and a light chain variable region sequence that is at least about 95%, 96%, 97%, 98%, 99% or 100% identical to SEQ ID NO: 13,
(iii) a heavy chain variable region sequence that is at least about 95%, 96%, 97%, 98%, 99% or 100% identical to SEQ ID NO: 17 and a light chain variable region sequence that is at least about 95%, 96%, 97%, 98%, 99% or 100% identical to SEQ ID NO: 13, or
(iv) a heavy chain variable region sequence that is at least about 95%, 96%, 97%, 98%, 99% or 100% identical to SEQ ID NO: 19 and a light chain variable region sequence that is at least about 95%, 96%, 97%, 98%, 99% or 100% identical to SEQ ID NO: 13.

47. The immune activating Fc domain binding molecule of embodiment 46, wherein the Fc domain binding moiety is capable of specific binding to an IgG1 Fc domain comprising the amino acid substitution P329G (numbering according to Kabat EU index), wherein the Fc domain binding moiety comprises the heavy chain variable region sequence of SEQ ID NO: 19 and the light chain variable of SEQ ID NO: 13.

48. The immune activating Fc domain binding molecule of any one of embodiments 1-20 and 25-40, wherein the Fc domain binding moiety is capable of specific binding to an IgG1 Fc domain comprising the amino acid mutations I253A, H310A and H435A (numbering according to Kabat EU index), wherein the Fc domain binding moiety comprises:
(i) a heavy chain variable region (VH) comprising
   (a) the heavy chain complementarity-determining region (CDR H) 1 amino acid sequence SYGMS (SEQ ID NO:168);
   (b) the CDR H2 amino acid sequence SSGGSY (SEQ ID NO:169); and
   (c) the CDR H3 amino acid sequence LGMITTGYAMDY (SEQ ID NO:170); and
(ii) a light chain variable region (VL) comprising
   (d) the light chain complementary-determining region (CDR L) 1 amino acid sequence RSSQTIVHSTGHTYLE (SEQ ID NO:171);
   (e) the CDR L2 amino acid sequence KVSNRFS (SEQ ID NO:172); and
   (f) the CDR L3 amino acid sequence ALWYSNHWV FQGSHVPYT (SEQ ID NO:173).

49. The immune activating Fc domain binding molecule of any one of embodiments 40-48, wherein the variable domains VL and VH of the Fab light chain and the Fab heavy chain of the Fc domain binding moiety are replaced by each other, or the variable domains VL and VH of the Fab light chain and the Fab heavy chain of the immune activating moiety are replaced by each other.

50. The immune activating Fc domain binding molecule of any one of embodiments 40-48, wherein the constant domains CL and CH1 of the Fab light chain and the Fab heavy chain of the Fc domain binding moiety are replaced by each other, or the constant domains CL and CH1 of the Fab light chain and the Fab heavy chain of the immune activating moiety are replaced by each other.

51. The immune activating Fc domain binding molecule of any one of embodiments 1-50, wherein the Fc domain binding moiety comprises a first Fab molecule and the immune activating moiety comprises a second Fab molecule.

52. The immune activating Fc domain binding molecule embodiment 51, wherein
i) in the constant domain CL of the first Fab molecule the amino acid at position 124 is substituted independently by lysine (K), arginine (R) or histidine (H) (numbering according to Kabat), and wherein in the constant domain CH1 of the first Fab molecule the amino acid at position 147 or the amino acid at position 213 is substituted independently by glutamic acid (E), or aspartic acid (D) (numbering according to Kabat EU index); or
ii) in the constant domain CL of the second Fab molecule the amino acid at position 124 is substituted independently by lysine (K), arginine (R) or histidine (H) (numbering according to Kabat), and wherein in the constant domain CH1 of the second Fab molecule the amino acid at position 147 or the amino acid at position 213 is substituted independently by glutamic acid (E), or aspartic acid (D) (numbering according to Kabat EU index).

53. The immune activating Fc domain binding molecule according to embodiment 51 or 52, wherein in the constant domain CL of the first Fab molecule under a) the amino acid at position 124 is substituted independently by lysine (K), arginine (R) or histidine (H) (numbering according to Kabat), and wherein in the constant domain CH1 of the first Fab molecule under a) the amino acid at position 147 or the amino acid at position 213 is substituted independently by glutamic acid (E), or aspartic acid (D) (numbering according to Kabat EU index).

54. The immune activating Fc domain binding molecule according to any one of embodiments 51-53, wherein in the constant domain CL of the first Fab molecule the amino acid at position 124 is substituted independently by lysine (K), arginine (R) or histidine (H) (numbering according to Kabat), and wherein in the constant domain CH1 of the first Fab molecule the amino acid at position 147 is substituted independently by glutamic acid (E), or aspartic acid (D) (numbering according to Kabat EU index).

55. The immune activating Fc domain binding molecule according to any one of embodiments 51-54, wherein in the constant domain CL of the first Fab molecule the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and wherein in the constant domain CH1 of the first Fab molecule the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

56. The immune activating Fc domain binding molecule according to embodiment 55, wherein in the constant domain CL of the first Fab molecule the amino acid at position 124 is substituted by lysine (K) (numbering according to Kabat) and the amino acid at position 123 is substituted by arginine (R) (numbering according to Kabat), and wherein in the constant domain CH1 of the first Fab molecule the amino acid at position 147 is substituted by glutamic acid (E) (numbering according to Kabat EU index) and the amino acid at position 213 is substituted by glutamic acid (E) (numbering according to Kabat EU index).

57. The immune activating Fc domain binding molecule according to embodiment 55, wherein in the constant domain CL of the first Fab molecule the amino acid at position 124 is substituted by lysine (K) (numbering according to Kabat) and the amino acid at position 123 is substituted by lysine (K) (numbering according to Kabat), and wherein in the constant domain CH1 of the first Fab molecule the amino acid at position 147 is substituted by glutamic acid (E) (numbering according to Kabat EU index) and the amino acid at position 213 is substituted by glutamic acid (E) (numbering according to Kabat EU index).

58. The immune activating Fc domain binding molecule according to any one of embodiments 51-57, further comprising
d) a third Fab molecule which specifically binds to the target Fc domain comprising the first set of at least one amino acid substitution.

59. The immune activating Fc domain binding molecule according to embodiment 58 wherein the third Fab molecule is identical to the first Fab molecule.

60. The immune activating Fc domain binding molecule of embodiment 58 or 59, wherein the third Fab comprises:
(i) a heavy chain variable region (VH) comprising
   (a) the heavy chain complementarity-determining region (CDR H) 1 amino acid sequence RYWMN (SEQ ID NO:1);
   (b) a CDR H2 amino acid sequence selected from the group consisting of EITPDSSTINYTPSLKD (SEQ ID NO:2), EITPDSSTINYTPSLKG (SEQ ID NO:11) and EITPDSSTINYAPSLKG (SEQ ID NO:16); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

61. The immune activating Fc domain binding molecule of embodiment 60, wherein the third Fab comprises:
(i) a heavy chain variable region (VH) comprising
   (a) the heavy chain complementarity-determining region (CDR H) 1 amino acid sequence RYWMN (SEQ ID NO:1);
   (b) the CDR H2 amino acid sequence EITPDSSTINYTPSLKD (SEQ ID NO:2); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

62. The immune activating Fc domain binding molecule of embodiment 60, wherein the third Fab comprises:
(i) a heavy chain variable region (VH) comprising
   (a) the heavy chain complementarity-determining region (CDR H) 1 amino acid sequence RYWMN (SEQ ID NO:1);
   (b) the CDR H2 amino acid sequence EITPDSSTINYTPSLKG (SEQ ID NO:11); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

63. The immune activating Fc domain binding molecule of embodiment 60, wherein the third Fab comprises:
(i) a heavy chain variable region (VH) comprising
   (a) the heavy chain complementarity-determining region (CDR H) 1 amino acid sequence RYWMN (SEQ ID NO:1);
   (b) the CDR H2 amino acid sequence EITPDSSTINYAPSLKG (SEQ ID NO:16); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

64. The immune activating Fc domain binding molecule of embodiment 58 or 59, wherein the third Fab comprises a heavy chain variable region sequence that is at least about 95%, 96%, 97%, 98%, 99% or 100% identical to an amino acid sequence selected from the group consisting of SEQ ID NO:7, SEQ ID NO:12, SEQ ID NO:17 and SEQ ID NO:19, and a light chain variable region sequence that is at least about 95%, 96%, 97%, 98%, 99% or 100% identical to an amino acid sequence selected from the group consisting of SEQ ID NO:8 and SEQ ID NO:13.

65. The immune activating Fc domain binding molecule of embodiment 64, wherein the third Fab comprises:
(i) a heavy chain variable region sequence that is at least about 95%, 96%, 97%, 98%, 99% or 100% identical to SEQ ID NO: 7 and a light chain variable region sequence that is at least about 95%, 96%, 97%, 98%, 99% or 100% identical to SEQ ID NO: 8,
(ii) a heavy chain variable region sequence that is at least about 95%, 96%, 97%, 98%, 99% or 100% identical to SEQ ID NO: 12 and a light chain variable region sequence that is at least about 95%, 96%, 97%, 98%, 99% or 100% identical to SEQ ID NO: 13,
(iii) a heavy chain variable region sequence that is at least about 95%, 96%, 97%, 98%, 99% or 100% identical to SEQ ID NO: 17 and a light chain variable region sequence that is at least about 95%, 96%, 97%, 98%, 99% or 100% identical to SEQ ID NO: 13, or
(iv) a heavy chain variable region sequence that is at least about 95%, 96%, 97%, 98%, 99% or 100% identical to SEQ ID NO: 19 and a light chain variable region sequence that is at least about 95%, 96%, 97%, 98%, 99% or 100% identical to SEQ ID NO: 13.

66. The immune activating Fc domain binding molecule of embodiment 64, wherein the third Fab comprises the heavy chain variable region sequence of SEQ ID NO: 19 and the light chain variable of SEQ ID NO: 13.

67. The immune activating Fc domain binding molecule of embodiment 58 or 59, wherein the third Fab comprises:
(i) a heavy chain variable region (VH) comprising
   (a) the heavy chain complementarity-determining region (CDR H) 1 amino acid sequence SYGMS (SEQ ID NO:168);
   (b) the CDR H2 amino acid sequence SSGGSY (SEQ ID NO:169); and
   (c) the CDR H3 amino acid sequence LGMITTGYAMDY (SEQ ID NO:170); and
(ii) a light chain variable region (VL) comprising
   (d) the light chain complementary-determining region (CDR L) 1 amino acid sequence RSSQTIVHSTGHTYLE (SEQ ID NO:171);
   (e) the CDR L2 amino acid sequence KVSNRFS (SEQ ID NO:172); and
   (f) the CDR L3 amino acid sequence ALWYSNHWV FQGSHVPYT (SEQ ID NO:173).

68. The immune activating Fc domain binding molecule according to any one of embodiments 47-67, wherein the first and the second Fab molecule are fused to each other, optionally via a peptide linker.

69. The immune activating Fc domain binding molecule according to any one of embodiments 47-68, wherein the second Fab molecule is fused at the C-terminus of the Fab heavy chain to the N-terminus of the Fab heavy chain of the first Fab molecule.

70. The immune activating Fc domain binding molecule of any one of embodiments 47-69, wherein the first Fab molecule is fused at the C-terminus of the Fab heavy chain to the N-terminus of the Fab heavy chain of the second Fab molecule.

71. The immune activating Fc domain binding molecule of embodiment 69 or 70, wherein the Fab light chain of the first Fab molecule and the Fab light chain of the second Fab molecule are fused to each other, optionally via a peptide linker.

72. The immune activating Fc domain binding molecule according to embodiment 47-71, wherein the second Fab molecule is fused at the C-terminus of the Fab heavy chain to the N-terminus of the first or the second subunit of the Fc domain.

73. The immune activating Fc domain binding molecule according to embodiment 47-71, wherein the first Fab molecule is fused at the C-terminus of the Fab heavy chain to the N-terminus of the first or the second subunit of the Fc domain.

74. The immune activating Fc domain binding molecule according to embodiment 47-71, wherein the first and the second Fab molecule are each fused at the C-terminus of the Fab heavy chain to the N-terminus of one of the subunits of the Fc domain.

75. The immune activating Fc domain binding molecule according to any one of embodiments 47-73, wherein the third Fab molecule is fused at the C-terminus of the Fab heavy chain to the N-terminus of the first or second subunit of the Fc domain.

76. The immune activating Fc domain binding molecule of embodiment 47-71, wherein the second and the third Fab molecule are each fused at the C-terminus of the Fab heavy chain to the N-terminus of one of the subunits of the Fc domain, and the first Fab molecule is fused at the C-terminus of the Fab heavy chain to the N-terminus of the Fab heavy chain of the second Fab molecule.

77. The immune activating Fc domain binding molecule according to embodiment 47-71, wherein the first and the third Fab molecule are each fused at the C-terminus of the Fab heavy chain to the N-terminus of one of the subunits of the Fc domain, and the second Fab molecule is fused at the C-terminus of the Fab heavy chain to the N-terminus of the Fab heavy chain of the first Fab molecule.

78. The immune activating Fc domain binding molecule of any one of embodiments 1-77, wherein the immune activating moiety is capable of specific binding to an activating T cell antigen.

79. The immune activating Fc domain binding molecule of embodiment 78, wherein the activating T cell antigen is CD3.

80. The immune activating Fc domain binding molecule of embodiment 78 or 79, wherein the activating T cell antigen is CD3 epsilon.

81. The immune activating Fc domain binding molecule of any one of embodiments 78-80, wherein the immune activating moiety specifically binds to an activating T cell antigen, particularly CD3, more particularly CD3 epsilon.

82. The immune activating Fc domain binding molecule of any one of embodiments 78-81, wherein the immune activating moiety is a Fab molecule.

83. An immune activating fragment crystallizable (Fc) domain binding molecule comprising
a) a first Fab molecule,
b) a second Fab molecule, wherein the variable domains VL and VH of the Fab light chain and the Fab heavy chain are replaced by each other, and
c) a half-life extending Fc domain composed of a first and a second subunit capable of stable association;
wherein
(i) the first Fab molecule specifically binds to a target Fc domain comprising a first set of at least one amino acid substitution and the second Fab molecule specifically binds to an activating T cell antigen, particularly CD3, more particularly CD3 epsilon, wherein the first Fab molecule does not specifically bind to the half-life extending Fc domain; or
(ii) the second Fab molecule specifically binds to a target Fc domain comprising a first set of at least one amino acid substitution and the first Fab molecule specifically binds to an activating T cell antigen, particularly CD3, more particularly CD3 epsilon, wherein the second Fab molecule does not specifically bind to the half-life extending Fc domain;
wherein in the constant domain CL of the first Fab molecule under a) the amino acid at position 124 is substituted by lysine (K) (numbering according to Kabat) and the amino acid at position 123 is substituted by arginine (R) or lysine (K) (numbering according to Kabat), and wherein in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index); and wherein the first Fab molecule under a) and the second Fab molecule under b) are each fused at the C-terminus of the Fab heavy chain to the N-terminus of one of the subunits of the Fc domain under c).

84. An immune activating fragment crystallizable (Fc) domain binding molecule comprising
a) a first Fab molecule which specifically binds to a target Fc domain comprising a first set of at least one amino acid substitution,
b) a second Fab molecule which specifically binds to an activating T cell antigen, particularly CD3, more particularly CD3 epsilon, and wherein the variable domains VL and VH of the Fab light chain and the Fab heavy chain are replaced by each other,
c) a third Fab molecule which specifically binds to the target Fc domain, and
d) a half-life extending Fc domain composed of a first and a second subunit capable of stable association,
wherein the first and second Fab molecules do not specifically bind to the half-life extending Fc domain;
wherein in the constant domain CL of the first Fab molecule under a) and the third Fab molecule under c) the amino acid at position 124 is substituted by lysine (K) (numbering according to Kabat) and the amino acid at position 123 is substituted by arginine (R) or lysine (K) (numbering according to Kabat), and wherein in the constant domain CH1 of the first Fab molecule under a) and the third Fab molecule under c) the amino acid at position 147 is substituted by glutamic acid (E) (numbering according to Kabat EU index) and the amino acid at position 213 is substituted by glutamic acid (E) (numbering according to Kabat EU index); and wherein
(i) the first Fab molecule under a) is fused at the C-terminus of the Fab heavy chain to the N-terminus of the Fab heavy chain of the second Fab molecule under b), and the second Fab molecule under b) and the third Fab molecule under c) are each fused at the C-terminus of the Fab heavy chain to the N-terminus of one of the subunits of the Fc domain under d), or
(ii) the second Fab molecule under b) is fused at the C-terminus of the Fab heavy chain to the N-terminus of the Fab heavy chain of the first Fab molecule under a), and the first Fab molecule under a) and the third Fab molecule under c) are each fused at the C-terminus of the Fab heavy chain to the N-terminus of one of the subunits of the Fc domain under d).

85. The immune activating Fc domain binding molecule molecule of any one of embodiments 82-84, wherein the activating T cell antigen is CD3, particularly CD3 epsilon, and the Fab molecule which specifically binds to the activating T cell antigen comprises the heavy chain complementarity determining region (CDR) 1 of SEQ ID NO: 35, the heavy chain CDR 2 of SEQ ID NO: 37, the heavy chain CDR 3 of SEQ ID NO: 43, the light chain CDR 1 of SEQ ID NO: 53, the light chain CDR 2 of SEQ ID NO: 54 and the light chain CDR 3 of SEQ ID NO: 55.

86. The immune activating Fc domain binding molecule molecule of any one of embodiments 82-85, wherein the activating T cell antigen is CD3, particulary CD3 epsilon, and the Fab molecule which specifically binds to the activating T cell antigen comprises a heavy chain variable region comprising an amino acid sequence that is at least about 95%, 96%, 97%, 98%, 99% or 100% identical to the amino acid sequence of SEQ ID NO: 49 and a light chain variable region comprising an amino acid sequence that is at least about 95%, 96%, 97%, 98%, 99% or 100% identical to the amino acid sequence of SEQ ID NO: 56.

87. The immune activating Fc domain binding molecule molecule of any one of embodiments 82-84, wherein the activating T cell antigen is CD3, particularly CD3 epsilon, and the Fab molecule which specifically binds to the activating T cell antigen comprises the heavy chain complementarity determining region (CDR) 1 of SEQ ID NO: 35, the heavy chain CDR 2 of SEQ ID NO: 33, the heavy chain CDR 3 of SEQ ID NO: 176, the light chain CDR 1 of SEQ ID NO: 53, the light chain CDR 2 of SEQ ID NO: 54 and the light chain CDR 3 of SEQ ID NO: 55.

88. The immune activating Fc domain binding molecule molecule of any one of embodiments 82-85, wherein the activating T cell antigen is CD3, particulary CD3 epsilon, and the Fab molecule which specifically binds to the activating T cell antigen comprises a heavy chain variable region comprising an amino acid sequence that is at least about 95%, 96%, 97%, 98%, 99% or 100% identical to the amino acid sequence of SEQ ID NO: 177 and a light chain variable region comprising an amino acid sequence that is at least about 95%, 96%, 97%, 98%, 99% or 100% identical to the amino acid sequence of SEQ ID NO: 56.

89. The immune activating Fc domain binding molecule molecule of any one of embodiments 82-84, wherein the activating T cell antigen is CD3, particularly CD3 epsilon, and the Fab molecule which specifically binds to the activating T cell antigen comprises the heavy chain complementarity determining region (CDR) 1 of SEQ ID NO: 34, the heavy chain CDR 2 of SEQ ID NO: 37, the heavy chain CDR 3 of SEQ ID NO: 41, the light chain CDR 1 of SEQ ID NO: 53, the light chain CDR 2 of SEQ ID NO: 54 and the light chain CDR 3 of SEQ ID NO: 55.

90. The immune activating Fc domain binding molecule molecule of any one of embodiments 82-85, wherein the activating T cell antigen is CD3, particulary CD3 epsilon, and the Fab molecule which specifically binds to the activating T cell antigen comprises a heavy chain variable region comprising an amino acid sequence that is at least about 95%, 96%, 97%, 98%, 99% or 100% identical to the amino acid sequence of SEQ ID NO: 47 and a light chain variable region comprising an amino acid sequence that is at least about 95%, 96%, 97%, 98%, 99% or 100% identical to the amino acid sequence of SEQ ID NO: 56.

91. The immune activating Fc domain binding molecule according to any one of embodiments 82-86, wherein the first Fab molecule and/or the third Fab molecule comprises:
(i) a heavy chain variable region (VH) comprising
   (a) the heavy chain complementarity-determining region (CDR H) 1 amino acid sequence RYWMN (SEQ ID NO:1);
   (b) a CDR H2 amino acid sequence selected from the group consisting of EITPDSSTINYTPSLKD (SEQ ID NO:2), EITPDSSTINYTPSLKG (SEQ ID NO:11) and EITPDSSTINYAPSLKG (SEQ ID NO:16); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

92. The immune activating Fc domain binding molecule according to any one of embodiments 82-86, wherein the first Fab molecule and/or the third Fab molecule comprises:
(i) a heavy chain variable region (VH) comprising
   (a) the heavy chain complementarity-determining region (CDR H) 1 amino acid sequence RYWMN (SEQ ID NO:1);
   (b) the CDR H2 amino acid sequence EITPDSSTINYAPSLKG (SEQ ID NO:16); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

93. The immune activating Fc domain binding molecule according to any one of embodiments 82-92, wherein the first Fab molecule and/or the third Fab molecule comprises a heavy chain variable region sequence that is at least about 95%, 96%, 97%, 98%, 99% or 100% identical to an amino acid sequence selected from the group consisting of SEQ ID NO:7, SEQ ID NO:12, SEQ ID NO:17 and SEQ ID NO:19, and a light chain variable region sequence that is at least about 95%, 96%, 97%, 98%, 99% or 100% identical to an amino acid sequence selected from the group consisting of SEQ ID NO:8 and SEQ ID NO:13.

94. The immune activating Fc domain binding molecule according to any one of embodiments 82-93, wherein the first Fab molecule and/or the third Fab molecule comprises:
(i) a heavy chain variable region sequence that is at least about 95%, 96%, 97%, 98%, 99% or 100% identical to SEQ ID NO: 7 and a light chain variable region sequence that is at least about 95%, 96%, 97%, 98%, 99% or 100% identical to SEQ ID NO: 8,
(ii) a heavy chain variable region sequence that is at least about 95%, 96%, 97%, 98%, 99% or 100% identical to SEQ ID NO: 12 and a light chain variable region sequence that is at least about 95%, 96%, 97%, 98%, 99% or 100% identical to SEQ ID NO: 13,
(iii) a heavy chain variable region sequence that is at least about 95%, 96%, 97%, 98%, 99% or 100% identical to SEQ ID NO: 17 and a light chain variable region sequence that is at least about 95%, 96%, 97%, 98%, 99% or 100% identical to SEQ ID NO: 13, or
(iv) a heavy chain variable region sequence that is at least about 95%, 96%, 97%, 98%, 99% or 100% identical to SEQ ID NO: 19 and a light chain variable region sequence that is at least about 95%, 96%, 97%, 98%, 99% or 100% identical to SEQ ID NO: 13.

95. The immune activating Fc domain binding molecule according to any one of embodiments 82-93, wherein the first Fab molecule and/or the third Fab molecule comprises the heavy chain variable region sequence of SEQ ID NO: 19 and the light chain variable of SEQ ID NO: 13.

96. An immune activating fragment crystallizable (Fc) domain binding molecule comprising
a) a first Fab molecule which specifically binds to a target Fc domain comprising a first set of at least one amino acid substitution;
b) a second Fab molecule which specifically binds to an activating T cell antigen, particularly CD3, more particularly CD3 epsilon, and wherein the variable domains VL and VH of the Fab light chain and the Fab heavy chain are replaced by each other;
c) a third Fab molecule which specifically binds to the target Fc domain; and
d) a half-life extending Fc domain composed of a first and a second subunit capable of stable association;
wherein
(i) the Fc domain binding moiety does not specifically bind to the half-life extending Fc domain;
(ii) the first Fab molecule under a) and the third Fab molecule under c) each comprise the heavy chain complementarity determining region (CDR) 1 of SEQ ID NO: 1, a heavy chain CDR 2 sequence selected from the group consisting of SEQ ID NO:2, SEQ ID NO:11 and SEQ ID NO:16, the heavy chain CDR 3 of SEQ ID NO: 3, the light chain CDR 1 of SEQ ID NO: 4, the light chain CDR 2 of SEQ ID NO: 5 and the light chain CDR 3 of SEQ ID NO: 6, and the second Fab molecule under b) comprises the heavy chain CDR 1 of SEQ ID NO: 35, the heavy chain CDR 2 of SEQ ID NO: 37, the heavy chain CDR 3 of SEQ ID NO: 43, the light chain CDR 1 of SEQ ID NO: 53, the light chain CDR 2 of SEQ ID NO: 54 and the light chain CDR 3 of SEQ ID NO: 55;
(iii) in the constant domain CL of the first Fab molecule under a) and the third Fab molecule under c) the amino acid at position 124 is substituted by lysine (K) (numbering according to Kabat) and the amino acid at position 123 is substituted by lysine (K) or arginine (R), particularly by arginine (R) (numbering according to Kabat), and wherein in the constant domain CH1 of the first Fab molecule under a) and the third Fab molecule under c) the amino acid at position 147 is substituted by glutamic acid (E) (numbering according to Kabat EU index) and the amino acid at position 213 is substituted by glutamic acid (E) (numbering according to Kabat EU index); and
(iv) the first Fab molecule under a) is fused at the C-terminus of the Fab heavy chain to the N-terminus of the Fab heavy chain of the second Fab molecule under b), and the second Fab molecule under b) and the third Fab molecule under c) are each fused at the C-terminus of the Fab heavy chain to the N-terminus of one of the subunits of the Fc domain under d).

97. The immune activating Fc domain binding molecule of embodiment 96, wherein the first Fab molecule under a) and the third Fab molecule under c) each comprise the heavy chain complementarity determining region (CDR) 1 of SEQ ID NO: 1, the heavy chain CDR 2 sequence of SEQ ID NO:16, the heavy chain CDR 3 of SEQ ID NO: 3, the light chain CDR 1 of SEQ ID NO: 4, the light chain CDR 2 of SEQ ID NO: 5 and the light chain CDR 3 of SEQ ID NO: 6.

98. The immune activating Fc domain binding molecule of embodiment 96 or 97, wherein the first Fab molecule under a) and the third Fab molecule under c) each comprise:
(i) a heavy chain variable region sequence that is at least about 95%, 96%, 97%, 98%, 99% or 100% identical to SEQ ID NO: 7 and a light chain variable region sequence that is at least about 95%, 96%, 97%, 98%, 99% or 100% identical to SEQ ID NO: 8,
(ii) a heavy chain variable region sequence that is at least about 95%, 96%, 97%, 98%, 99% or 100% identical to SEQ ID NO: 12 and a light chain variable region sequence that is at least about 95%, 96%, 97%, 98%, 99% or 100% identical to SEQ ID NO: 13,
(iii) a heavy chain variable region sequence that is at least about 95%, 96%, 97%, 98%, 99% or 100% identical to SEQ ID NO: 17 and a light chain variable region sequence that is at least about 95%, 96%, 97%, 98%, 99% or 100% identical to SEQ ID NO: 13, or
(iv) a heavy chain variable region sequence that is at least about 95%, 96%, 97%, 98%, 99% or 100% identical to SEQ ID NO: 19 and a light chain variable region sequence that is at least about 95%, 96%, 97%, 98%, 99% or 100% identical to SEQ ID NO: 13.

99. The immune activating Fc domain binding molecule of embodiment 96 or 97, wherein the first Fab molecule under a) and the third Fab molecule under c) each comprise the heavy chain variable region sequence of SEQ ID NO: 19 and the light chain variable of SEQ ID NO: 13.

100. The immune activating Fc domain binding molecule of embodiment 96-99, wherein the second Fab molecule under b) comprises a heavy chain variable region comprising the amino acid sequence of SEQ ID NO: 49 and a light chain variable region comprising the amino acid sequence of SEQ ID NO: 56.

101. The immune activating Fc domain binding molecule of embodiment 78-100, wherein the half-life extending Fc domain comprises a substitution at position P329 (numbering according to Kabat EU index) by an amino acid selected from the list consisting of arginine (R), leucine (L), isoleucine (I), and alanine (A)s.

102. The immune activating Fc domain binding molecule of any one of embodiments 1-77, wherein the immune activating moiety is capable of specific binding to a costimulatory T cell antigen.

103. The immune activating Fc domain binding molecule of embodiment 102, wherein the costimulatory T cell antigen is CD28.

104. The immune activating Fc domain binding molecule of embodiment 102 or 103, wherein the immune activating moiety specifically binds to a costimulatory T cell antigen.

105. The immune activating Fc domain binding molecule of any one of embodiments 102-104, wherein the immune activating moiety specifically binds to CD28.

106. The immune activating Fc domain binding molecule of any one of embodiments 102-105, wherein the immune activating moiety is a Fab molecule.

107. An immune activating fragment crystallizable (Fc) domain binding molecule comprising
a) a first Fab molecule,
b) a second Fab molecule, wherein the variable domains VL and VH of the Fab light chain and the Fab heavy chain are replaced by each other, and
c) a half-life extending Fc domain composed of a first and a second subunit capable of stable association;
wherein
(i) the first Fab molecule specifically binds to a target Fc domain comprising a first set of at least one amino acid substitution and the second Fab molecule specifically binds to a costimulatory T cell antigen, particularly CD28, wherein the first Fab molecule does not specifically bind to the half-life extending Fc domain; or
(ii) the second Fab molecule specifically binds to a target Fc domain comprising a first set of at least one amino acid substitution and the first Fab molecule specifically binds to a costimulatory T cell antigen, particularly CD28, wherein the second Fab molecule does not specifically bind to the half-life extending Fc domain;
wherein in the constant domain CL of the first Fab molecule under a) the amino acid at position 124 is substituted by lysine (K) (numbering according to Kabat) and the amino acid at position 123 is substituted by arginine (R) or lysine (K) (numbering according to Kabat), and wherein in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index); and
wherein the first Fab molecule under a) and the second Fab molecule under b) are each fused at the C-terminus of the Fab heavy chain to the N-terminus of one of the subunits of the Fc domain under c).

108. An immune activating fragment crystallizable (Fc) domain binding molecule comprising
a) a first Fab molecule which specifically binds to a target Fc domain comprising a first set of at least one amino acid substitution,
b) a second Fab molecule which specifically binds to a costimulatory T cell antigen, particularly CD28, and wherein the variable domains VL and VH of the Fab light chain and the Fab heavy chain are replaced by each other,
c) a third Fab molecule which specifically binds to the target Fc domain, and
d) a half-life extending Fc domain composed of a first and a second subunit capable of stable association, wherein the first and second Fab molecules do not specifically bind to the half-life extending Fc domain;
wherein in the constant domain CL of the first Fab molecule under a) and the third Fab molecule under c) the amino acid at position 124 is substituted by lysine (K) (numbering according to Kabat) and the amino acid at position 123 is substituted by arginine (R) or lysine (K) (numbering according to Kabat), and wherein in the constant domain CH1 of the first Fab molecule under a) and the third Fab molecule under c) the amino acid at position 147 is substituted by glutamic acid (E) (numbering according to Kabat EU index) and the amino acid at position 213 is substituted by glutamic acid (E) (numbering according to Kabat EU index); and
wherein
(i) the first Fab molecule under a) is fused at the C-terminus of the Fab heavy chain to the N-terminus of the Fab heavy chain of the second Fab molecule under b), and the second Fab molecule under b) and the third Fab molecule under c) are each fused at the C-terminus of the Fab heavy chain to the N-terminus of one of the subunits of the Fc domain under d), or
(ii) the second Fab molecule under b) is fused at the C-terminus of the Fab heavy chain to the N-terminus of the Fab heavy chain of the first Fab molecule under a), and the first Fab molecule under a) and the third Fab molecule under c) are each fused at the C-terminus of the Fab heavy chain to the N-terminus of one of the subunits of the Fc domain under d).

109. The immune activating Fc domain binding molecule molecule of any one of embodiments 106-108, wherein Fab molecule which specifically binds to CD28 comprises the heavy chain complementarity determining region (CDR) 1 of SEQ ID NO: 94, the heavy chain CDR 2 of SEQ ID NO: 95, the heavy chain CDR 3 of SEQ ID NO: 96, the light chain CDR 1 of SEQ ID NO: 97, the light chain CDR 2 of SEQ ID NO: 98 and the light chain CDR 3 of SEQ ID NO: 99; or the heavy chain complementarity determining region (CDR) 1 of SEQ ID NO: 94, the heavy chain CDR 2 of SEQ ID NO: 95, the heavy chain CDR 3 of SEQ ID NO: 102, the light chain CDR 1 of SEQ ID NO: 103, the light chain CDR 2 of SEQ ID NO: 98 and the light chain CDR 3 of SEQ ID NO: 99

110. The immune activating Fc domain binding molecule molecule of any one of embodiments 106-109, wherein the Fab molecule which specifically binds CD28 comprises a heavy chain variable region comprising an amino acid sequence that is at least about 95%, 96%, 97%, 98%, 99% or 100% identical to the amino acid sequence of SEQ ID NO: 100 and a light chain variable region comprising an amino acid sequence that is at least about 95%, 96%, 97%, 98%, 99% or 100% identical to the amino acid sequence of SEQ ID NO: 101; or an amino acid sequence that is at least about 95%, 96%, 97%, 98%, 99% or 100% identical to the amino acid sequence of SEQ ID NO: 104 and a light chain variable region comprising an amino acid sequence that is at least about 95%, 96%, 97%, 98%, 99% or 100% identical to the amino acid sequence of SEQ ID NO: 105.

111. The immune activating Fc domain binding molecule according to any one of embodiments 106-110, wherein the first Fab molecule and/or the third Fab molecule comprises:
(i) a heavy chain variable region (VH) comprising
   (a) the heavy chain complementarity-determining region (CDR H) 1 amino acid sequence RYWMN (SEQ ID NO:1);
   (b) a CDR H2 amino acid sequence selected from the group consisting of EITPDSSTINYTPSLKD (SEQ ID NO:2), EITPDSSTINYTPSLKG (SEQ ID NO:11) and EITPDSSTINYAPSLKG (SEQ ID NO:16); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

112. The immune activating Fc domain binding molecule according to any one of embodiments 106-110, wherein the first Fab molecule and/or the third Fab molecule comprises:
(i) a heavy chain variable region (VH) comprising
   (a) the heavy chain complementarity-determining region (CDR H) 1 amino acid sequence RYWMN (SEQ ID NO:1);
   (b) the CDR H2 amino acid sequence EITPDSSTINYAPSLKG (SEQ ID NO:16); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

113. The immune activating Fc domain binding molecule according to any one of embodiments 106-112, wherein the first Fab molecule and/or the third Fab molecule comprises a heavy chain variable region sequence that is at least about 95%, 96%, 97%, 98%, 99% or 100% identical to an amino acid sequence selected from the group consisting of SEQ ID NO:7, SEQ ID NO:12, SEQ ID NO:17 and SEQ ID NO:19, and a light chain variable region sequence that is at least about 95%, 96%, 97%, 98%, 99% or 100% identical to an amino acid sequence selected from the group consisting of SEQ ID NO:8 and SEQ ID NO:13.

114. The immune activating Fc domain binding molecule according to any one of embodiments 106-113, wherein the first Fab molecule and/or the third Fab molecule comprises:
(i) a heavy chain variable region sequence that is at least about 95%, 96%, 97%, 98%, 99% or 100% identical to SEQ ID NO: 7 and a light chain variable region sequence that is at least about 95%, 96%, 97%, 98%, 99% or 100% identical to SEQ ID NO: 8,
(ii) a heavy chain variable region sequence that is at least about 95%, 96%, 97%, 98%, 99% or 100% identical to SEQ ID NO: 12 and a light chain variable region sequence that is at least about 95%, 96%, 97%, 98%, 99% or 100% identical to SEQ ID NO: 13,
(iii) a heavy chain variable region sequence that is at least about 95%, 96%, 97%, 98%, 99% or 100% identical to SEQ ID NO: 17 and a light chain variable region sequence that is at least about 95%, 96%, 97%, 98%, 99% or 100% identical to SEQ ID NO: 13, or
(iv) a heavy chain variable region sequence that is at least about 95%, 96%, 97%, 98%, 99% or 100% identical to SEQ ID NO: 19 and a light chain variable region sequence that is at least about 95%, 96%, 97%, 98%, 99% or 100% identical to SEQ ID NO: 13.

115. The immune activating Fc domain binding molecule according to any one of embodiments 106-113, wherein the first Fab molecule and/or the third Fab molecule comprises the heavy chain variable region sequence of SEQ ID NO: 19 and the light chain variable of SEQ ID NO: 13.

116. An immune activating fragment crystallizable (Fc) domain binding molecule comprising
a) a first Fab molecule which specifically binds to a target Fc domain comprising a first set of at least one amino acid substitution;
b) a second Fab molecule which specifically binds to a costimulatory T cell antigen, particularly CD28, and wherein the variable domains VL and VH of the Fab light chain and the Fab heavy chain are replaced by each other;
c) a third Fab molecule which specifically binds to the target Fc domain; and
d) a half-life extending Fc domain composed of a first and a second subunit capable of stable association;
wherein
(i) the Fc domain binding moiety does not specifically bind to the half-life extending Fc domain; (ii) the first Fab molecule under a) and the third Fab molecule under c) each comprise the heavy chain complementarity determining region (CDR) 1 of SEQ ID NO: 1, a heavy chain CDR 2 sequence selected from the group consisting of SEQ ID NO:2, SEQ ID NO:11 and SEQ ID NO:16, the heavy chain CDR 3 of SEQ ID NO: 3, the light chain CDR 1 of SEQ ID NO: 4, the light chain CDR 2 of SEQ ID NO: 5 and the light chain CDR 3 of SEQ ID NO: 6, and the second Fab molecule under b) comprises the heavy chain CDR 1 of SEQ ID NO: 94, the heavy chain CDR 2 of SEQ ID NO: 95, the heavy chain CDR 3 of SEQ ID NO: 96, the light chain CDR 1 of SEQ ID NO: 97, the light chain CDR 2 of SEQ ID NO: 98 and the light chain CDR 3 of SEQ ID NO: 99; or the heavy chain CDR 1 of SEQ ID NO: 94, the heavy chain CDR 2 of SEQ ID NO: 95, the heavy chain CDR 3 of SEQ ID NO: 102, the light chain CDR 1 of SEQ ID NO: 103, the light chain CDR 2 of SEQ ID NO: 98 and the light chain CDR 3 of SEQ ID NO: 99;
(iii) in the constant domain CL of the first Fab molecule under a) and the third Fab molecule under c) the amino acid at position 124 is substituted by lysine (K) (numbering according to Kabat) and the amino acid at position 123 is substituted by lysine (K) or arginine (R), particularly by arginine (R) (numbering according to Kabat), and wherein in the constant domain CH1 of the first Fab molecule under a) and the third Fab molecule under c) the amino acid at position 147 is substituted by glutamic acid (E) (numbering according to Kabat EU index) and the amino acid at position 213 is substituted by glutamic acid (E) (numbering according to Kabat EU index); and
(iv) the first Fab molecule under a) is fused at the C-terminus of the Fab heavy chain to the N-terminus of the Fab heavy chain of the second Fab molecule under b), and the second Fab molecule under b) and the third Fab molecule under c) are each fused at the C-terminus of the Fab heavy chain to the N-terminus of one of the subunits of the Fc domain under d).

117. The immune activating Fc domain binding molecule of embodiment 116, wherein the first Fab molecule under a) and the third Fab molecule under c) each comprise the heavy chain complementarity determining region (CDR) 1 of SEQ ID NO: 1, the heavy chain CDR 2 sequence of SEQ ID NO:16, the heavy chain CDR 3 of SEQ ID NO: 3, the light chain CDR 1 of SEQ ID NO: 4, the light chain CDR 2 of SEQ ID NO: 5 and the light chain CDR 3 of SEQ ID NO: 6.

118. The immune activating Fc domain binding molecule of embodiment 116 or 117, wherein the first Fab molecule under a) and the third Fab molecule under c) each comprise:
(i) a heavy chain variable region sequence that is at least about 95%, 96%, 97%, 98%, 99% or 100% identical to SEQ ID NO: 7 and a light chain variable region sequence that is at least about 95%, 96%, 97%, 98%, 99% or 100% identical to SEQ ID NO: 8,
(ii) a heavy chain variable region sequence that is at least about 95%, 96%, 97%, 98%, 99% or 100% identical to SEQ ID NO: 12 and a light chain variable region sequence that is at least about 95%, 96%, 97%, 98%, 99% or 100% identical to SEQ ID NO: 13,
(iii) a heavy chain variable region sequence that is at least about 95%, 96%, 97%, 98%, 99% or 100% identical to SEQ ID NO: 17 and a light chain variable region sequence that is at least about 95%, 96%, 97%, 98%, 99% or 100% identical to SEQ ID NO: 13, or
(iv) a heavy chain variable region sequence that is at least about 95%, 96%, 97%, 98%, 99% or 100% identical to SEQ ID NO: 19 and a light chain variable region sequence that is at least about 95%, 96%, 97%, 98%, 99% or 100% identical to SEQ ID NO: 13.

119. The immune activating Fc domain binding molecule of embodiment 116 or 117, wherein the first Fab molecule under a) and the third Fab molecule under c) each comprise the heavy chain variable region sequence of SEQ ID NO: 19 and the light chain variable of SEQ ID NO: 13.

120. The immune activating Fc domain binding molecule of embodiment 116-119, wherein the second Fab molecule under b) comprises a heavy chain variable region comprising the amino acid sequence of SEQ ID NO: 100 and a light chain variable region comprising the amino acid sequence of SEQ ID NO:101, or a heavy chain variable region comprising the amino acid sequence of SEQ ID NO: 104 and a light chain variable region comprising the amino acid sequence of SEQ ID NO: 105.

121. The immune activating Fc domain binding molecule of embodiment 102-120, wherein the half-life extending Fc domain comprises a substitution at position P329 (numbering according to Kabat EU index) by an amino acid selected from the list consisting of arginine (R), leucine (L), isoleucine (I), and alanine (A).

122. The immune activating Fc domain binding molecule of any one of embodiments 1-77, wherein the immune activating moiety is capable of specific binding to a costimulatory T cell antigen.

123. The immune activating Fc domain binding molecule of embodiment 122, wherein the costimulatory T cell antigen is 4-1BB.

124. The immune activating Fc domain binding molecule of embodiment 122 or 123, wherein the immune activating moiety specifically binds to a costimulatory T cell antigen.

125. The immune activating Fc domain binding molecule of any one of embodiments 122-124, wherein the immune activating moiety specifically binds to 4-1BB.

126. The immune activating Fc domain binding molecule of any one of embodiments 122-125, wherein the immune activating moiety is a Fab molecule.

127. An immune activating fragment crystallizable (Fc) domain binding molecule comprising
a) a first Fab molecule,
b) a second Fab molecule, wherein the variable domains VL and VH of the Fab light chain and the Fab heavy chain are replaced by each other, and
c) a half-life extending Fc domain composed of a first and a second subunit capable of stable association;
wherein
(i) the first Fab molecule specifically binds to a target Fc domain comprising a first set of at least one amino acid substitution and the second Fab molecule specifically binds to a costimulatory T cell antigen, particularly 4-1BB, wherein the first Fab molecule does not specifically bind to the half-life extending Fc domain; or
(ii) the second Fab molecule specifically binds to a target Fc domain comprising a first set of at least one amino acid substitution and the first Fab molecule specifically binds to a costimulatory T cell antigen, particularly 4-1BB, wherein the second Fab molecule does not specifically bind to the half-life extending Fc domain;
wherein in the constant domain CL of the first Fab molecule under a) the amino acid at position 124 is substituted by lysine (K) (numbering according to Kabat) and the amino acid at position 123 is substituted by arginine (R) or lysine (K) (numbering according to Kabat), and wherein in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index); and
wherein the first Fab molecule under a) and the second Fab molecule under b) are each fused at the C-terminus of the Fab heavy chain to the N-terminus of one of the subunits of the Fc domain under c).

128. An immune activating fragment crystallizable (Fc) domain binding molecule comprising
a) a first Fab molecule which specifically binds to a target Fc domain comprising a first set of at least one amino acid substitution,
b) a second Fab molecule which specifically binds to a costimulatory T cell antigen, particularly 4-1BB, and wherein the variable domains VL and VH of the Fab light chain and the Fab heavy chain are replaced by each other,
c) a third Fab molecule which specifically binds to the target Fc domain, and
d) a half-life extending Fc domain composed of a first and a second subunit capable of stable association,
wherein the first and second Fab molecules do not specifically bind to the half-life extending Fc domain;
wherein in the constant domain CL of the first Fab molecule under a) and the third Fab molecule under c) the amino acid at position 124 is substituted by lysine (K) (numbering according to Kabat) and the amino acid at position 123 is substituted by arginine (R) or lysine (K) (numbering according to Kabat), and wherein in the constant domain CH1 of the first Fab molecule under a) and the third Fab molecule under c) the amino acid at position 147 is substituted by glutamic acid (E) (numbering according to Kabat EU index) and the amino acid at position 213 is substituted by glutamic acid (E) (numbering according to Kabat EU index); and wherein
(i) the first Fab molecule under a) is fused at the C-terminus of the Fab heavy chain to the N-terminus of the Fab heavy chain of the second Fab molecule under b), and the second Fab molecule under b) and the third Fab molecule under c) are each fused at the C-terminus of the Fab heavy chain to the N-terminus of one of the subunits of the Fc domain under d), or
(ii) the second Fab molecule under b) is fused at the C-terminus of the Fab heavy chain to the N-terminus of the Fab heavy chain of the first Fab molecule under a), and the first Fab molecule under a) and the third Fab molecule under c) are each fused at the C-terminus of the Fab heavy chain to the N-terminus of one of the subunits of the Fc domain under d).

129. The immune activating Fc domain binding molecule molecule of any one of embodiments 126-129, wherein Fab molecule which specifically binds to 4-1BB comprises the heavy chain complementarity determining region (CDR) 1 of SEQ ID NO: 133, the heavy chain CDR 2 of SEQ ID NO: 134, the heavy chain CDR 3 of SEQ ID NO: 135, the light chain CDR 1 of SEQ ID NO: 136, the light chain CDR 2 of SEQ ID NO: 137 and the light chain CDR 3 of SEQ ID NO: 138.

130. The immune activating Fc domain binding molecule molecule of any one of embodiments 126-129, wherein the Fab molecule which specifically binds to 4-1BB comprises a heavy chain variable region comprising i) an amino acid sequence that is at least about 95%, 96%, 97%, 98%, 99% or 100% identical to the amino acid sequence of SEQ ID NO: 139 and a light chain variable region comprising an amino acid sequence that is at least about 95%, 96%, 97%, 98%, 99% or 100% identical to the amino acid sequence of SEQ ID NO: 140,

131. The immune activating Fc domain binding molecule according to any one of embodiments 126-130, wherein the first Fab molecule and/or the third Fab molecule comprises:
(i) a heavy chain variable region (VH) comprising
   (a) the heavy chain complementarity-determining region (CDR H) 1 amino acid sequence RYWMN (SEQ ID NO:1);
   (b) a CDR H2 amino acid sequence selected from the group consisting of EITPDSSTINYTPSLKD (SEQ ID NO:2), EITPDSSTINYTPSLKG (SEQ ID NO:11) and EITPDSSTINYAPSLKG (SEQ ID NO:16); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

132. The immune activating Fc domain binding molecule according to any one of embodiments 126-140, wherein the first Fab molecule and/or the third Fab molecule comprises:
(i) a heavy chain variable region (VH) comprising
   (a) the heavy chain complementarity-determining region (CDR H) 1 amino acid sequence RYWMN (SEQ ID NO:1);
   (b) the CDR H2 amino acid sequence EITPDSSTINYAPSLKG (SEQ ID NO:16); and
   (c) the CDR H3 amino acid sequence PYDYGAWFAS (SEQ ID NO:3); and
(ii) a light chain variable region (VL) comprising
   (d) the light chain complementary-determining region (CDR L) 1 amino acid sequence RSSTGAVTTSNYAN (SEQ ID NO:4);
   (e) the CDR L2 amino acid sequence GTNKRAP (SEQ ID NO:5); and
   (f) the CDR L3 amino acid sequence ALWYSNHWV (SEQ ID NO:6).

133. The immune activating Fc domain binding molecule according to any one of embodiments 126-132, wherein the first Fab molecule and/or the third Fab molecule comprises a heavy chain variable region sequence that is at least about 95%, 96%, 97%, 98%, 99% or 100% identical to an amino acid sequence selected from the group consisting of SEQ ID NO:7, SEQ ID NO:12, SEQ ID NO:17 and SEQ ID NO:19, and a light chain variable region sequence that is at least about 95%, 96%, 97%, 98%, 99% or 100% identical to an amino acid sequence selected from the group consisting of SEQ ID NO:8 and SEQ ID NO:13.

134. The immune activating Fc domain binding molecule according to any one of embodiments 116-133, wherein the first Fab molecule and/or the third Fab molecule comprises:
(i) a heavy chain variable region sequence that is at least about 95%, 96%, 97%, 98%, 99% or 100% identical to SEQ ID NO: 7 and a light chain variable region sequence that is at least about 95%, 96%, 97%, 98%, 99% or 100% identical to SEQ ID NO: 8,
(ii) a heavy chain variable region sequence that is at least about 95%, 96%, 97%, 98%, 99% or 100% identical to SEQ ID NO: 12 and a light chain variable region sequence that is at least about 95%, 96%, 97%, 98%, 99% or 100% identical to SEQ ID NO: 13,
(iii) a heavy chain variable region sequence that is at least about 95%, 96%, 97%, 98%, 99% or 100% identical to SEQ ID NO: 17 and a light chain variable region sequence that is at least about 95%, 96%, 97%, 98%, 99% or 100% identical to SEQ ID NO: 13, or
(iv) a heavy chain variable region sequence that is at least about 95%, 96%, 97%, 98%, 99% or 100% identical to SEQ ID NO: 19 and a light chain variable region sequence that is at least about 95%, 96%, 97%, 98%, 99% or 100% identical to SEQ ID NO: 13.

135. The immune activating Fc domain binding molecule according to any one of embodiments 126-133, wherein the first Fab molecule and/or the third Fab molecule comprises the heavy chain variable region sequence of SEQ ID NO: 19 and the light chain variable of SEQ ID NO: 13.

136. An immune activating fragment crystallizable (Fc) domain binding molecule comprising
a) a first Fab molecule which specifically binds to a target Fc domain comprising a first set of at least one amino acid substitution;
b) a second Fab molecule which specifically binds to an costimulatory T cell antigen, particularly 4-1BB, and wherein the variable domains VL and VH of the Fab light chain and the Fab heavy chain are replaced by each other;
c) a third Fab molecule which specifically binds to the target Fc domain; and d) a half-life extending Fc domain composed of a first and a second subunit capable of stable association;
wherein
(i) the Fc domain binding moiety does not specifically bind to the half-life extending Fc domain;
(ii) the first Fab molecule under a) and the third Fab molecule under c) each comprise the heavy chain complementarity determining region (CDR) 1 of SEQ ID NO: 1, a heavy chain CDR 2 sequence selected from the group consisting of SEQ ID NO:2, SEQ ID NO:11 and SEQ ID NO:16, the heavy chain CDR 3 of SEQ ID NO: 3, the light chain CDR 1 of SEQ ID NO: 4, the light chain CDR 2 of SEQ ID NO: 5 and the light chain CDR 3 of SEQ ID NO: 6, and the second Fab molecule under b) comprises the heavy chain CDR 1 of SEQ ID NO: 133, the heavy chain CDR 2 of SEQ ID NO: 134, the heavy chain CDR 3 of SEQ ID NO: 135, the light chain CDR 1 of SEQ ID NO: 136, the light chain CDR 2 of SEQ ID NO: 137 and the light chain CDR 3 of SEQ ID NO: 138;
(iii) in the constant domain CL of the first Fab molecule under a) and the third Fab molecule under c) the amino acid at position 124 is substituted by lysine (K) (numbering according to Kabat) and the amino acid at position 123 is substituted by lysine (K) or arginine (R), particularly by arginine (R) (numbering according to Kabat), and wherein in the constant domain CH1 of the first Fab molecule under a) and the third Fab molecule under c) the amino acid at position 147 is substituted by glutamic acid (E) (numbering according to Kabat EU index) and the amino acid at position 213 is substituted by glutamic acid (E) (numbering according to Kabat EU index); and
(iv) the first Fab molecule under a) is fused at the C-terminus of the Fab heavy chain to the N-terminus of the Fab heavy chain of the second Fab molecule under b), and the second Fab molecule under b) and the third Fab molecule under c) are each fused at the C-terminus of the Fab heavy chain to the N-terminus of one of the subunits of the Fc domain under d).

137. The immune activating Fc domain binding molecule of embodiment 136, wherein the first Fab molecule under a) and the third Fab molecule under c) each comprise the heavy chain complementarity determining region (CDR) 1 of SEQ ID NO: 1, the heavy chain CDR 2 sequence of SEQ ID NO:16, the heavy chain CDR 3 of SEQ ID NO: 3, the light chain CDR 1 of SEQ ID NO: 4, the light chain CDR 2 of SEQ ID NO: 5 and the light chain CDR 3 of SEQ ID NO: 6.

138. The immune activating Fc domain binding molecule of embodiment 136 or 137, wherein the first Fab molecule under a) and the third Fab molecule under c) each comprise:
(i) a heavy chain variable region sequence that is at least about 95%, 96%, 97%, 98%, 99% or 100% identical to SEQ ID NO: 7 and a light chain variable region sequence that is at least about 95%, 96%, 97%, 98%, 99% or 100% identical to SEQ ID NO: 8,
(ii) a heavy chain variable region sequence that is at least about 95%, 96%, 97%, 98%, 99% or 100% identical to SEQ ID NO: 12 and a light chain variable region sequence that is at least about 95%, 96%, 97%, 98%, 99% or 100% identical to SEQ ID NO: 13,
(iii) a heavy chain variable region sequence that is at least about 95%, 96%, 97%, 98%, 99% or 100% identical to SEQ ID NO: 17 and a light chain variable region sequence that is at least about 95%, 96%, 97%, 98%, 99% or 100% identical to SEQ ID NO: 13, or
(iv) a heavy chain variable region sequence that is at least about 95%, 96%, 97%, 98%, 99% or 100% identical to SEQ ID NO: 19 and a light chain variable region sequence that is at least about 95%, 96%, 97%, 98%, 99% or 100% identical to SEQ ID NO: 13.

139. The immune activating Fc domain binding molecule of embodiment 136 or 137, wherein the first Fab molecule under a) and the third Fab molecule under c) each comprise the heavy chain variable region sequence of SEQ ID NO: 19 and the light chain variable of SEQ ID NO: 13.

140. The immune activating Fc domain binding molecule of embodiment 136-139, wherein the second Fab molecule under b) comprises a heavy chain variable region comprising the amino acid sequence of SEQ ID NO: 139 and a light chain variable region comprising the amino acid sequence of SEQ ID NO:140.

141. The immune activating Fc domain binding molecule of embodiment 122-140, wherein the half-life extending Fc domain comprises a substitution at position P329 (numbering according to Kabat EU index) by an amino acid selected from the list consisting of arginine (R), leucine (L), isoleucine (I), and alanine (A).

142. The immune activating fragment crystallizable (Fc) domain binding molecule of any one of embodiments 1-48, wherein the immune activating moiety is a cytokine.

143. The immune activating fragment crystallizable (Fc) domain binding molecule of embodiment 142, wherein the cytokine is selected from the group consisting of IL2, IL7, IL15, IL18, IFNa and IFNg.

144. The immune activating fragment crystallizable (Fc) domain binding molecule of embodiment 142 or 143, wherein the immune activating moiety is a mutant interleukin-2 (IL-2) polypeptide.

145. The immune activating fragment crystallizable (Fc) domain binding molecule of embodiment 105, wherein the mutant IL-2 polypeptide is a human IL-2 molecule comprising the amino acid substitutions F42A, Y45A and L72G (numbering relative to the human IL-2 sequence SEQ ID NO:166).

146. The immune activating fragment crystallizable (Fc) domain binding molecule of embodiment 145, wherein the mutant IL-2 polypeptide further comprises the amino acid substitution T3A and/or the amino acid substitution C125A.

147. The immune activating fragment crystallizable (Fc) domain binding molecule of any one of embodiments 144-146, wherein the mutant IL-2 polypeptide comprises an amino acid sequence that is at least about 95%, 96%, 97%, 98%, 99% or 100% identical to the amino acid sequence of SEQ ID NO:167, wherein the mutant IL-2 polypeptide shows reduced affinity to the high-affinity IL-2 receptor and substantially similar affinity to the intermediate affinity IL-2 receptor, each compared to a wild-type IL-2 polypeptide.

148. The immune activating fragment crystallizable (Fe) domain binding molecule of any one of embodiments 144-147, wherein the mutant IL-2 polypeptide comprises the amino acid sequence of SEQ ID NO:167.

149. The immune activating fragment crystallizable (Fc) domain binding molecule of any one of embodiments 143-148, wherein the Fc domain binding moiety comprises a Fab molecule.

150. The immune activating Fc domain binding molecule of embodiment 149, wherein the Fab comprises a heavy chain variable region sequence that is at least about 95%, 96%, 97%, 98%, 99% or 100% identical to an amino acid sequence selected from the group consisting of SEQ ID NO:7, SEQ ID NO:12, SEQ ID NO:17 and SEQ ID NO:19, and a light chain variable region sequence that is at least about 95%, 96%, 97%, 98%, 99% or 100% identical to an amino acid sequence selected from the group consisting of SEQ ID NO:8 and SEQ ID NO:13.

151. The immune activating Fc domain binding molecule of embodiment 149, wherein the Fab comprises:
(i) a heavy chain variable region sequence that is at least about 95%, 96%, 97%, 98%, 99% or 100% identical to SEQ ID NO: 7 and a light chain variable region sequence that is at least about 95%, 96%, 97%, 98%, 99% or 100% identical to SEQ ID NO: 8,
(ii) a heavy chain variable region sequence that is at least about 95%, 96%, 97%, 98%, 99% or 100% identical to SEQ ID NO: 12 and a light chain variable region sequence that is at least about 95%, 96%, 97%, 98%, 99% or 100% identical to SEQ ID NO: 13,
(iii) a heavy chain variable region sequence that is at least about 95%, 96%, 97%, 98%, 99% or 100% identical to SEQ ID NO: 17 and a light chain variable region sequence that is at least about 95%, 96%, 97%, 98%, 99% or 100% identical to SEQ ID NO: 13, or
(iv) a heavy chain variable region sequence that is at least about 95%, 96%, 97%, 98%, 99% or 100% identical to SEQ ID NO: 19 and a light chain variable region sequence that is at least about 95%, 96%, 97%, 98%, 99% or 100% identical to SEQ ID NO: 13.

152. The immune activating Fc domain binding molecule of embodiment 151, wherein the Fab comprises the heavy chain variable region sequence of SEQ ID NO: 19 and the light chain variable of SEQ ID NO: 13.

153. The immune activating Fc domain binding molecule of any one of embodiments 1-44, wherein the immune activating moiety comprises three ectodomains of 4-1BBL or a fragment thereof.

154. The immune activating Fc domain binding molecule of any one of embodiments 1-44, wherein the immune activating moiety comprises a first and a second polypeptide, wherein the first polypeptide contains a first heavy chain constant (CH1) or a light chain constant (CL) domain and the second polypeptide contains a CL or CH1 domain, respectively, wherein the second polypeptide is linked to the first polypeptide by a disulfide bond between the CH1 and CL domain, and wherein the first polypeptide comprises two ectodomains of 4-1BBL or a fragment thereof that are connected to each other and to the CH1 or CL domain by a peptide linker and wherein the second polypeptide comprises one ectodomain of said 4-1BBL or a fragment thereof connected via a peptide linker to the CL or CH1 domain of said polypeptide.

155. The immune activating Fc domain binding molecule of embodiment 153 or 154, wherein the ectodomain of 4-1BBL or a fragment thereof comprises the amino acid sequence selected from the group consisting of SEQ ID NO:117, SEQ ID NO: 118, SEQ ID NO:119, SEQ ID NO:120, SEQ ID NO:121, SEQ ID NO: 122, SEQ ID NO:123 and SEQ ID NO:124, particularly the amino acid sequence of SEQ ID NO:117 or SEQ ID NO:121.

156. The immune activating Fc domain binding molecule of any one of embodiments 153-155, wherein the immune activating moiety comprises a first and a second polypeptide that are linked to each other by a disulfide bond, wherein the antigen binding molecule is characterized in that the first polypeptide comprises the amino acid sequence selected from the group consisting of SEQ ID NO:125, SEQ ID NO:126, SEQ ID NO:127 and SEQ ID NO:128 and in that the second polypeptide comprises the amino acid sequence selected from the group consisting of SEQ ID NO:117, SEQ ID NO:121, SEQ ID NO:119 and SEQ ID NO:120.

157. The immune activating Fc domain binding molecule of any one of embodiments 1-48 or 153-156, wherein the molecule comprises a first heavy chain and a first light chain comprising the Fc domain binding moiety, in particular a Fab molecule capable of specific binding to the target Fc domain, and a second heavy chain and a second light chain comprising the immune activating moiety, wherein the second heavy chain comprises the first polypeptide comprising two ectodomains of 4-1BBL or a fragment thereof that are connected to each other and to the CH1 or CL domain by a peptide and the second light chain comprises the second polypeptide comprising one ectodomain of said 4-1BBL or a fragment thereof connected via a peptide linker to the CL or CH1 domain of said polypeptide, respectively.

158. The immune activating Fc domain binding molecule of any one of embodiments 154-157, wherein the first peptide comprising two ectodomains of 4-1BBL or a fragment thereof connected to each other by a first peptide linker is fused at its C-terminus by a second peptide linker to a CL domain that is part of a heavy chain, and the second peptide comprising one ectodomain of said 4-1BBL or a fragment thereof is fused at its C-terminus by a third peptide linker to a CH1 domain that is part of a light chain.

159. The immune activating Fc domain binding molecule of any one of embodiments 153-158, wherein the Fc domain binding moiety comprises a Fab molecule.

160. The immune activating Fc domain binding molecule of embodiment 159, wherein the Fab comprises a heavy chain variable region sequence that is at least about 95%, 96%, 97%, 98%, 99% or 100% identical to an amino acid sequence selected from the group consisting of SEQ ID NO:7, SEQ ID NO:12, SEQ ID NO:17 and SEQ ID NO:19, and a light chain variable region sequence that is at least about 95%, 96%, 97%, 98%, 99% or 100% identical to an amino acid sequence selected from the group consisting of SEQ ID NO:8 and SEQ ID NO:13.

161. The immune activating Fc domain binding molecule of embodiment 159 or 160, wherein the Fab comprises:
(i) a heavy chain variable region sequence that is at least about 95%, 96%, 97%, 98%, 99% or 100% identical to SEQ ID NO: 7 and a light chain variable region sequence that is at least about 95%, 96%, 97%, 98%, 99% or 100% identical to SEQ ID NO: 8,
(ii) a heavy chain variable region sequence that is at least about 95%, 96%, 97%, 98%, 99% or 100% identical to SEQ ID NO: 12 and a light chain variable region sequence that is at least about 95%, 96%, 97%, 98%, 99% or 100% identical to SEQ ID NO: 13,
(iii) a heavy chain variable region sequence that is at least about 95%, 96%, 97%, 98%, 99% or 100% identical to SEQ ID NO: 17 and a light chain variable region sequence that is at least about 95%, 96%, 97%, 98%, 99% or 100% identical to SEQ ID NO: 13, or
(iv) a heavy chain variable region sequence that is at least about 95%, 96%, 97%, 98%, 99% or 100% identical to SEQ ID NO: 19 and a light chain variable region sequence that is at least about 95%, 96%, 97%, 98%, 99% or 100% identical to SEQ ID NO: 13.

162. The immune activating Fc domain binding molecule of embodiment 158 or 159, wherein the Fab comprises the heavy chain variable region sequence of SEQ ID NO: 19 and the light chain variable of SEQ ID NO: 13.

163. The immune activating Fc domain binding molecule of any one of embodiments 1-70, wherein the immune activating moiety is capable of specific binding to an Fc receptor.

164. The immune activating Fc domain binding molecule of embodiment 163, wherein the Fc receptor is a Fc gamma receptor, in particular the FcgRIIIa receptor.

165. The immune activating Fc domain binding molecule of embodiment 163 or 164, wherein the Fc receptor is CD16.

166. One or more isolated polynucleotide encoding the immune activating Fc domain binding molecule of any one of embodiments 1 to 165.

167. One or more vector, particularly expression vector, comprising the polynucleotide(s) of embodiment 166.

168. A host cell comprising the polynucleotide(s) of embodiment 166 or the vector(s) of embodiment 167.

169. A method of producing an immune activating fragment crystallizable (Fc) domain binding molecule, comprising the steps of a) culturing the host cell of embodiment 168 under conditions suitable for the expression of the immune activating Fc domain binding molecule and b) recovering the immune activating Fc domain binding molecule.

170. A immune activating fragment crystallizable (Fc) domain binding molecule produced by the method of embodiment 169.

171. A pharmaceutical composition comprising the immune activating Fc domain binding molecule of any one of embodiments 1 to 165 and a pharmaceutically acceptable carrier.

172. The immune activating Fc domain binding molecule of any one of embodiments 1 to 165 or the pharmaceutical composition of embodiment 171 for use as a medicament.

173. The immune activating Fc domain binding molecule of any one of embodiments 1 to 165 or the pharmaceutical composition of embodiment 171 for use in the treatment of a disease in an individual in need thereof.

174. The immune activating Fc domain binding molecule or the pharmaceutical composition of for use according to embodiment 173, wherein the disease is cancer.

175. The immune activating Fc domain binding molecule of any one of embodiments 1 to 165 or the pharmaceutical composition of embodiment 171 for use in the treatment of a disease in an individual in need thereof, wherein the immune activating Fc domain binding molecule is used in combination with a targeting antibody comprising the target Fc domain.

176. The immune activating Fc domain binding molecule or the pharmaceutical composition of for use according to embodiment 175, wherein the disease is cancer.

177. The immune activating Fc domain binding molecule of embodiment 175 or 176, wherein the targeting antibody is capable of specific binding to a target antigen, in particular a target antigen on a cancer cell.

178. Use of the immune activating Fc domain binding molecule of any one of embodiments 1 to 165 for the manufacture of a medicament for the treatment of a disease in an individual in need thereof.

179. A method of treating a disease in an individual, comprising administering to said individual a therapeutically effective amount of a composition comprising the immune activating Fc domain binding molecule of any one of embodiments 1 to 165 in a pharmaceutically acceptable form.

180. The use of embodiment 178 or the method of embodiment 179, wherein said disease is cancer.

181. A method of treating a disease in an individual, comprising
(a) administering to said individual a therapeutically effective amount of a composition comprising the immune activating Fc domain binding molecule of any one of embodiments 1 to 165 in a pharmaceutically acceptable form; and
(b) administering to said individual a therapeutically effective amount of a composition comprising a targeting antibody comprising the target Fc domain.

182. The method of embodiment 181, wherein the disease is cancer.

183. The method of embodiment 181 or 182, wherein the targeting antibody is capable of specific binding to a target antigen, in particular a target antigen on a cancer cell.

184. The method of any one of embodiments 181-183 wherein immune activating Fc domain binding molecules is administered before, at the same time, or after the antibody comprising the target Fc domain.

185. A method for inducing lysis of a cell, comprising contacting the cell with the immune activating Fc domain binding molecule of any one of embodiments 165 and with a targeting antibody comprising the target Fc domain in the presence of a T cell, wherein the targeting antibody is capable of specific binding to an antigen on the cell.

186. The invention as described hereinbefore.

Exemplary Sequences
anti-P329G antibodies
CDR definition according to Kabat
Anti-P329G (M-1.7.24) huIgG1

| | | |
|---|---|---|
| HCDR1 | RYWMN | 1 |
| HCDR2 | EITPDSSTINYTPSLKD | 2 |
| HCDR3 | PYDYGAWFAS | 3 |
| LCDR1 | RSSTGAVTTSNYAN | 4 |
| LCDR2 | GTNKRAP | 5 |
| LCDR3 | ALWYSNHWV | 6 |
| VH | EVKLLESGGGLVQPGGSLKLSCAASGFDFSRYWMNWVRQ | 7 |
| | APGKGLEWIGEITPDSSTINYTPSLKDKFIISRDNAKNTLYLQ | |
| | MIKVRSEDTALYYCVRPYDYGAWFASWGQGTLVTVSA | |
| VL | QAVVTQESALTTSPGETVTLTCRSSTGAVTTSNYANWVQE | 8 |
| | KPDHLFTGLIGGTNKRAPGVPARFSGSLIGDKAALTITGAQ | |
| | TEDEAIYFCALWYSNHWVFGGGTKLTVL | |
| HC | EVKLLESGGGLVQPGGSLKLSCAASGFDFSRYWMNWVRQ | 9 |
| | APGKGLEWIGEITPDSSTINYTPSLKDKFIISRDNAKNTLYLQ | |
| | MIKVRSEDTALYYCVRPYDYGAWFASWGQGTLVTVSAAS | |
| | TKGPSVFPLAPSSKSTSGGTAALGCLVKDYFPEPVTVSWNS | |
| | GALTSGVHTFPAVLQSSGLYSLSSVVTVPSSSLGTQTYICNV | |
| | NHKPSNTKVDKKVEPKSCDKTHTCPPCPAPELLGGPSVFLF | |
| | PPKPKDTLMISRTPEVTCVVVDVSHEDPEVKFNWYVDGVE | |
| | VHNAKTKPREEQYNSTYRVVSVLTVLHQDWLNGKEYKCK | |
| | VSNKALPAPIEKTISKAKGQPREPQVYTLPPSRDELTKNQVS | |
| | LTCLVKGFYPSDIAVEWESNGQPENNYKTTPPVLDSDGSFF | |
| | LYSKLTVDKSRWQQGNVFSCSVMHEALHNHYTQKSLSLSP | |
| LC | QAVVTQESALTTSPGETVTLTCRSSTGAVTTSNYANWVQE | 10 |
| | KPDHLFTGLIGGTNKRAPGVPARFSGSLIGDKAALTITGAQ | |
| | TEDEAIYFCALWYSNHWVFGGGTKLTVLGQPKAAPSVTLF | |
| | PPSSEELQANKATLVCLISDFYPGAVTVAWKADSSPVKAG | |
| | VETTTPSKQSNNKYAASSYLSLTPEQWKSHRSYSCQVTHE | |
| | GSTVEKTVAPTECS | |

Anti-P32G (VH1VL1) huIgG1

| | | |
|---|---|---|
| HCDR1 | RYWMN | 1 |
| HCDR2 | EITPDSSTINYTPSLKG | 11 |
| HCDR3 | PYDYGAWFAS | 3 |
| LCDR1 | RSSTGAVTTSNYAN | 4 |
| LCDR2 | GTNKRAP | 5 |
| LCDR3 | ALWYSNHWV | 6 |
| VH | EVQLVESGGGLVQPGGSLRLSCAASGFDFSRYWMNWVRQ | 12 |
| | APGKGLEWVGEITPDSSTINYTPSLKGRFTISRDNAKNSLYL | |
| | QMNSLRAEDTAVYYCVRPYDYGAWFASWGQGTLVTVSS | |
| VL | QAVVTQEPSLTVSPGGTVTLTCRSSTGAVTTSNYANWVQE | 13 |
| | KPDHLFTGLIGGTNKRAPGTPARFSGSLLGGKAALTLSGAQ | |
| | PEDEAEYYCALWYSNHWVFGGGTKLTVL | |
| HC | EVQLVESGGGLVQPGGSLRLSCAASGFDFSRYWMNWVRQ | 14 |
| | APGKGLEWVGEITPDSSTINYTPSLKGRFTISRDNAKNSLYL | |
| | QMNSLRAEDTAVYYCVRPYDYGAWFASWGQGTLVTVSS | |
| | ASTKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKKVEPKSCDKTHTCPPCPAPELLGGPSVF | |
| | LFPPKPKDTLMISRTPEVTCVVVDVSHEDPEVKFNWYVDG | |
| | VEVHNAKTKPREEQYNSTYRVVSVLTVLHQDWLNGKEYK | |
| | CKVSNKALPAPIEKTISKAKGQPREPQVYTLPPSRDELTKN | |
| | QVSLTCLVKGFYPSDIAVEWESNGQPENNYKTTPPVLDSD | |
| | GSFFLYSKLTVDKSRWQQGNVFSCSVMHEALHNHYTQKS | |
| | LSLSP | |
| LC | QAVVTQEPSLTVSPGGTVTLTCRSSTGAVTTSNYANWVQE | 15 |
| | KPDHLFTGLIGGTNKRAPGTPARFSGSLLGGKAALTLSGAQ | |
| | PEDEAEYYCALWYSNHWVFGGGTKLTVLGQPKAAPSVTL | |
| | FPPSSEELQANKATLVCLISDFYPGAVTVAWKADSSPVKAG | |
| | VETTTPSKQSNNKYAASSYLSLTPEQWKSHRSYSCQVTHE | |
| | GSTVEKTVAPTECS | |

Anti-P329G (VH2VL1) huIgG1

| | | |
|---|---|---|
| HCDR1 | RYWMN | 1 |
| HCDR2 | EITPDSSTINYAPSLKG | 16 |
| HCDR3 | PYDYGAWFAS | 3 |
| LCDR1 | RSSTGAVTTSNYAN | 4 |
| LCDR2 | GTNKRAP | 5 |
| LCDR3 | ALWYSNHWV | 6 |
| VH | EVQLVESGGGLVQPGGSLRLSCAASGFDFSRYWMNWVRQ | 17 |
| | APGKGLEWVGEITPDSSTINYAPSLKGRFTISRDNAKNSLYL | |
| | QMNSLRAEDTAVYYCVRPYDYGAWFASWGQGTLVTVSS | |
| VL | QAVVTQEPSLTVSPGGTVTLTCRSSTGAVTTSNYANWVQE | 13 |
| | KPDHLFTGLIGGTNKRAPGTPARFSGSLLGGKAALTLSGAQ | |
| | PEDEAEYYCALWYSNHWVFGGGTKLTVL | |
| HC | EVQLVESGGGLVQPGGSLRLSCAASGFDFSRYWMNWVRQ | 18 |
| | APGKGLEWVGEITPDSSTINYAPSLKGRFTISRDNAKNSLYL | |
| | QMNSLRAEDTAVYYCVRPYDYGAWFASWGQGTLVTVSS | |
| | ASTKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKKVEPKSCDKTHTCPPCPAPELLGGPSVF | |
| | LFPPKPKDTLMISRTPEVTCVVVDVSHEDPEVKFNWYVDG | |
| | VEVHNAKTKPREEQYNSTYRVVSVLTVLHQDWLNGKEYK | |
| | CKVSNKALPAPIEKTISKAKGQPREPQVYTLPPSRDELTKN | |
| | QVSLTCLVKGFYPSDIAVEWESNGQPENNYKTTPPVLDSD | |
| | GSFFLYSKLTVDKSRWQQGNVFSCSVMHEALHNHYTQKS | |
| | LSLSP | |
| LC | QAVVTQEPSLTVSPGGTVTLTCRSSTGAVTTSNYANWVQE | 15 |
| | KPDHLFTGLIGGTNKRAPGTPARFSGSLLGGKAALTLSGAQ | |
| | PEDEAEYYCALWYSNHWVFGGGTKLTVLGQPKAAPSVTL | |
| | FPPSSEELQANKATLVCLISDFYPGAVTVAWKADSSPVKAG | |
| | VETTTPSKQSNNKYAASSYLSLTPEQWKSHRSYSCQVTHE | |
| | GSTVEKTVAPTECS | |

Anti-P329G (VH3VL1) huIgG1

| | | |
|---|---|---|
| HCDR1 | RYWMN | 1 |
| HCDR2 | EITPDSSTINYAPSLKG | 16 |
| HCDR3 | PYDYGAWFAS | 3 |
| LCDR1 | RSSTGAVTTSNYAN | 4 |
| LCDR2 | GTNKRAP | 5 |
| LCDR3 | ALWYSNHWV | 6 |
| VH | EVQLVESGGGLVQPGGSLRLSCAASGFTFSRYWMNWVRQ | 19 |
| | APGKGLEWVGEITPDSSTINYAPSLKGRFTISRDNAKNSLYL | |
| | QMNSLRAEDTAVYYCARPYDYGAWFASWGQGTLVTVSS | |
| VL | QAVVTQEPSLTVSPGGTVTLTCRSSTGAVTTSNYANWVQE | 13 |
| | KPDHLFTGLIGGTNKRAPGTPARFSGSLLGGKAALTLSGAQ | |
| | PEDEAEYYCALWYSNHWVFGGGTKLTVL | |
| HC | EVQLVESGGGLVQPGGSLRLSCAASGFTFSRYWMNWVRQ | 20 |
| | APGKGLEWVGEITPDSSTINYAPSLKGRFTISRDNAKNSLYL | |
| | QMNSLRAEDTAVYYCARPYDYGAWFASWGQGTLVTVSS | |
| | ASTKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKKVEPKSCDKTHTCPPCPAPELLGGPSVF | |
| | LFPPKPKDTLMISRTPEVTCVVVDVSHEDPEVKFNWYVDG | |
| | VEVHNAKTKPREEQYNSTYRVVSVLTVLHQDWLNGKEYK | |
| | CKVSNKALPAPIEKTISKAKGQPREPQVYTLPPSRDELTKN | |
| | QVSLTCLVKGFYPSDIAVEWESNGQPENNYKTTPPVLDSD | |
| | GSFFLYSKLTVDKSRWQQGNVFSCSVMHEALHNHYTQKS | |
| | LSLSP | |
| LC | QAVVTQEPSLTVSPGGTVTLTCRSSTGAVTTSNYANWVQE | 15 |
| | KPDHLFTGLIGGTNKRAPGTPARFSGSLLGGKAALTLSGAQ | |
| | PEDEAEYYCALWYSNHWVFGGGTKLTVLGQPKAAPSVTL | |
| | FPPSSEELQANKATLVCLISDFYPGAVTVAWKADSSPVKAG | |
| | VETTTPSKQSNNKYAASSYLSLTPEQWKSHRSYSCQVTHE | |
| | GSTVEKTVAPTECS | |

Anti-P329G (VH4VL1) hulgG1

| | | |
|---|---|---|
| HCDR1 | RYWMN | 1 |
| HCDR2 | EITPDSSTINYADSVKG | 21 |
| HCDR3 | PYDYGAWFAS | 3 |
| LCDR1 | RSSTGAVTTSNYAN | 4 |
| LCDR2 | GTNKRAP | 5 |
| LCDR3 | ALWYSNHWV | 6 |
| VH | EVQLVESGGGLVQPGGSLRLSCAASGFDFSRYWMNWVRQ | 22 |
| | APGKGLEWVSEITPDSSTINYADSVKGRFTISRDNAKNSLY | |
| | LQMNSLRAEDTAVYYCARPYDYGAWFASWGQGTLVTVSS | |
| VL | QAVVTQEPSLTVSPGGTVTLTCRSSTGAVTTSNYANWVQE | 13 |
| | KPDHLFTGLIGGTNKRAPGTPARFSGSLLGGKAALTLSGAQ | |
| | PEDEAEYYCALWYSNHWVFGGGTKLTVL | |
| HC | EVQLVESGGGLVQPGGSLRLSCAASGFDFSRYWMNWVRQ | 23 |
| | APGKGLEWVSEITPDSSTINYADSVKGRFTISRDNAKNSLY | |
| | LQMNSLRAEDTAVYYCARPYDYGAWFASWGQGTLVTVSS | |
| | ASTKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKKVEPKSCDKTHTCPPCPAPELLGGPSVF | |
| | LFPPKPKDTLMISRTPEVTCVVVDVSHEDPEVKFNWYVDG | |
| | VEVHNAKTKPREEQYNSTYRVVSVLTVLHQDWLNGKEYK | |
| | CKVSNKALPAPIEKTISKAKGQPREPQVYTLPPSRDELTKN | |
| | QVSLTCLVKGFYPSDIAVEWESNGQPENNYKTTPPVLDSD | |
| | GSFFLYSKLTVDKSRWQQGNVFSCSVMHEALHNHYTQKS | |
| | LSLSP | |
| LC | QAVVTQEPSLTVSPGGTVTLTCRSSTGAVTTSNYANWVQE | 15 |
| | KPDHLFTGLIGGTNKRAPGTPARFSGSLLGGKAALTLSGAQ | |
| | PEDEAEYYCALWYSNHWVFGGGTKLTVLGQPKAAPSVTL | |
| | FPPSSEELQANKATLVCLISDFYPGAVTVAWKADSSPVKAG | |
| | VETTTPSKQSNNKYAASSYLSLTPEQWKSHRSYSCQVTHE | |
| | GSTVEKTVAPTECS | |

Anti-P329G (VH1VL2) huIgG1

| | | |
|---|---|---|
| HCDR1 | RYWMN | 1 |
| HCDR2 | EITPDSSTINYTPSLKG | 11 |
| HCDR3 | PYDYGAWFAS | 3 |
| LCDR1 | RSSTGAVTTSNYAN | 4 |
| LCDR2 | GTNKRAP | 5 |
| LCDR3 | ALWYSNHWV | 6 |
| VH | EVQLVESGGGLVQPGGSLRLSCAASGFDFSRYWMNWVRQ | 12 |
| | APGKGLEWVGEITPDSSTINYTPSLKGRFTISRDNAKNSLYL | |
| | QMNSLRAEDTAVYYCVRPYDYGAWFASWGQGTLVTVSS | |
| VL | QAVVTQEPSLTVSPGGTVTLTCRSSTGAVTTSNYANWFQQ | 24 |
| | KPGQAFTGLIGGTNKRAPGTPARFSGSLLGGKAALTLSGAQ | |
| | PEDEAEYYCALWYSNHWVFGGGTKLTVL | |
| HC | EVQLVESGGGLVQPGGSLRLSCAASGFDFSRYWMNWVRQ | 14 |
| | APGKGLEWVGEITPDSSTINYTPSLKGRFTISRDNAKNSLYL | |
| | QMNSLRAEDTAVYYCVRPYDYGAWFASWGQGTLVTVSS | |
| | ASTKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKKVEPKSCDKTHTCPPCPAPELLGGPSVF | |
| | LFPPKPKDTLMISRTPEVTCVVVDVSHEDPEVKFNWYVDG | |
| | VEVHNAKTKPREEQYNSTYRVVSVLTVLHQDWLNGKEYK | |
| | CKVSNKALPAPIEKTISKAKGQPREPQVYTLPPSRDELTKN | |
| | QVSLTCLVKGFYPSDIAVEWESNGQPENNYKTTPPVLDSD | |
| | GSFFLYSKLTVDKSRWQQGNVFSCSVMHEALHNHYTQKS | |
| | LSLSP | |
| LC | QAVVTQEPSLTVSPGGTVTLTCRSSTGAVTTSNYANWFQQ | 25 |
| | KPGQAFTGLIGGTNKRAPGTPARFSGSLLGGKAALTLSGAQ | |
| | PEDEAEYYCALWYSNHWVFGGGTKLTVLGQPKAAPSVTL | |
| | FPPSSEELQANKATLVCLISDFYPGAVTVAWKADSSPVKAG | |
| | VETTTPSKQSNNKYAASSYLSLTPEQWKSHRSYSCQVTHE | |
| | GSTVEKTVAPTECS | |

Anti-P329G (VH1VL3) huIgG1

| | | |
|---|---|---|
| HCDR1 | RYWMN | 1 |
| HCDR2 | EITPDSSTINYTPSLKG | 11 |
| HCDR3 | PYDYGAWFAS | 3 |
| LCDR1 | GSSTGAVTTSNYAN | 26 |
| LCDR2 | GTNKRAP | 5 |
| LCDR3 | ALWYSNHWV | 6 |
| VH | EVQLVESGGGLVQPGGSLRLSCAASGFDFSRYWMNWVRQ | 12 |
| | APGKGLEWVGEITPDSSTINYTPSLKGRFTISRDNAKNSLYL | |
| | QMNSLRAEDTAVYYCVRPYDYGAWFASWGQGTLVTVSS | |
| VL | QAVVTQEPSLTVSPGGTVTLTCGSSTGAVTTSNYANWFQQ | 27 |
| | KPGQAPRTLIGGTNKRAPGTPARFSGSLLGGKAALTLSGAQ | |
| | PEDEAEYYCALWYSNHWVFGGGTKLTVL | |
| HC | EVQLVESGGGLVQPGGSLRLSCAASGFDFSRYWMNWVRQ | 14 |
| | APGKGLEWVGEITPDSSTINYTPSLKGRFTISRDNAKNSLYL | |
| | QMNSLRAEDTAVYYCVRPYDYGAWFASWGQGTLVTVSS | |
| | ASTKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKKVEPKSCDKTHTCPPCPAPELLGGPSVF | |
| | LFPPKPKDTLMISRTPEVTCVVVDVSHEDPEVKFNWYVDG | |
| | VEVHNAKTKPREEQYNSTYRVVSVLTVLHQDWLNGKEYK | |
| | CKVSNKALPAPIEKTISKAKGQPREPQVYTLPPSRDELTKN | |
| | QVSLTCLVKGFYPSDIAVEWESNGQPENNYKTTPPVLDSD | |
| | GSFFLYSKLTVDKSRWQQGNVFSCSVMHEALHNHYTQKS | |
| | LSLSP | |
| LC | QAVVTQEPSLTVSPGGTVTLTCGSSTGAVTTSNYANWFQQ | 28 |
| | KPGQAPRTLIGGTNKRAPGTPARFSGSLLGGKAALTLSGAQ | |
| | PEDEAEYYCALWYSNHWVFGGGTKLTVLGQPKAAPSVTL | |
| | FPPSSEELQANKATLVCLISDFYPGAVTVAWKADSSPVKAG | |
| | VETTTPSKQSNNKYAASSYLSLTPEQWKSHRSYSCQVTHE | |
| | GSTVEKTVAPTECS | |

P329 IgG1 Fc variants

| | | |
|---|---|---|
| huIgG1 Fc | EPKSCDKTHTCPPCPAPEAAGGPSVFLFPPKPKDTLMISRTP | 29 |
| P329G | EVTCVVVDVSHEDPEVKFNWYVDGVEVHNAKTKPREEQY | |
| | NSTYRVVSVLTVLHQDWLNGKEYKCKVSNKALGAPIEKTI | |
| | SKAKGQPREPQVCTLPPSRDELTKNQVSLSCAVKGFYPSDI | |
| | AVEWESNGQPENNYKTTPPVLDSDGSFFLVSKLTVDKSRW | |
| | QQGNVFSCSVMHEALHNHYTQKSLSLSP | |
| huIgG1 Fc | EPKSCDKTHTCPPCPAPEAAGGPSVFLFPPKPKDTLMISRTP | 30 |
| P329L | EVTCVVVDVSHEDPEVKFNWYVDGVEVHNAKTKPREEQY | |
| | NSTYRVVSVLTVLHQDWLNGKEYKCKVSNKALLAPIEKTI | |
| | SKAKGQPREPQVYTLPPSRDELTKNQVSLTCLVKGFYPSDI | |
| | AVEWESNGQPENNYKTTPPVLDSDGSFFLYSKLTVDKSRW | |
| | QQGNVFSCSVMHEALHNHYTQKSLSLSP | |
| huIgG1 Fc | EPKSCDKTHTCPPCPAPEAAGGPSVFLFPPKPKDTLMISRTP | 31 |
| P329I | EVTCVVVDVSHEDPEVKFNWYVDGVEVHNAKTKPREEQY | |
| | NSTYRVVSVLTVLHQDWLNGKEYKCKVSNKALIAPIEKTIS | |
| | KAKGQPREPQVYTLPPSRDELTKNQVSLTCLVKGFYPSDIA | |
| | VEWESNGQPENNYKTTPPVLDSDGSFFLYSKLTVDKSRWQ | |
| | QGNVFSCSVMHEALHNHYTQKSLSLSP | |
| huIgG1 Fc | EPKSCDKTHTCPPCPAPEAAGGPSVFLFPPKPKDTLMISRTP | 32 |
| P329R | EVTCVVVDVSHEDPEVKFNWYVDGVEVHNAKTKPREEQY | |
| | NSTYRVVSVLTVLHQDWLNGKEYKCKVSNKALRAPIEKTI | |
| | SKAKGQPREPQVYTLPPSRDELTKNQVSLTCLVKGFYPSDI | |
| | AVEWESNGQPENNYKTTPPVLDSDGSFFLYSKLTVDKSRW | |
| | QQGNVFSCSVMHEALHNHYTQKSLSLSP | |
| huIgG1 Fc | EPKSCDKTHTCPPCPAPEAAGGPSVFLFPPKPKDTLMISRTP | 33 |
| P329A | EVTCVVVDVSHEDPEVKFNWYVDGVEVHNAKTKPREEQY | |
| | NSTYRVVSVLTVLHQDWLNGKEYKCKVSNKALAAPIEKTI | |
| | SKAKGQPREPQVYTLPPSRDELTKNQVSLTCLVKGFYPSDI | |
| | AVEWESNGQPENNYKTTPPVLDSDGSFFLYSKLTVDKSRW | |
| | QQGNVFSCSVMHEALHNHYTQKSLSLSP | |

Improved CD3 binder

| | | |
|---|---|---|
| CD3_{orig} | TYAMN | 34 |
| HCDR1 | | |
| CD3ₒₚₜ | SYAMN | 35 |
| HCDR1 | | |
| (P033.078) | | |
| (P035.093) | | |
| (P021.045) | | |
| CD3ₒₚₜ | NYAMN | 36 |
| HCDR1 | | |
| (P035.064) | | |
| (P004.042) | | |
| CD3_{orig} | RIRSKYNNYATYYADSVKG | 37 |
| HCDR2, | | |
| CD3ₒₚₜ | | |
| HCDR2 | | |
| (P035.093) | | |
| (P021.045) | | |
| CD3ₒₚₜ | RIRSKYNEYATYYADSVKG | 38 |
| HCDR2 | | |
| (P033.078) | | |
| CD3ₒₚₜ | RIRSKHNGYATYYADSVKG | 39 |
| HCDR2 | | |
| (P035.064) | | |
| CD3ₒₚₜ | RIRTKYNEYATYYADSVKG | 40 |
| HCDR2 | | |
| (P004.042) | | |
| CD3_{orig} | HGNFGNSYVSWFAY | 41 |
| HCDR3 | | |
| CD3ₒₚₜ | ASNFPSSFVSYFGY | 42 |
| HCDR3 | | |
| (P033.078) | | |
| CD3ₒₚₜ | ASNFPASYVSYFAY | 43 |
| HCDR3 | | |
| (P035.093) | | |
| CD3ₒₚₜ | ASNFPSSYVSYFGY | 44 |
| HCDR3 | | |
| (P035.064) | | |
| CD3ₒₚₜ | ASNFPSSYVSYFAY | 45 |
| HCDR3 | | |
| (P021.045) | | |
| CD3ₒₚₜ | ASNFPQSYVSYFGY | 46 |
| HCDR3 | | |
| (P004.042) | | |
| CD3orig VH | EVQLLESGGGLVQPGGSLRLSCAASGFTFSTYAMNWVRQA | 47 |
| | PGKGLEWVSRIRSKYNNYATYYADSVKGRFTISRDDSKNT | |
| | LYLQMNSLRAEDTAVYYCVRHGNFGNSYVSWFAYWGQG | |
| | TLVTVSS | |
| CD3ₒₚₜ VH | EVQLLESGGGLVQPGGSLRLSCAASGFTFESYAMNWVRQA | 48 |
| (P033.078) | PGKGLEWVSRIRSKYNEYATYYADSVKGRFTI | |
| | SRDDSKNTLYLQMNSLRAEDTAVYYCVRASNFPSSFVSYF | |
| | GYWGQGTLVTVSS | |
| CD3ₒₚₜ VH | EVQLLESGGGLVQPGGSLRLSCAASGFTFSSYAMNWVRQA | 49 |
| (P035.093) | PGKGLEWVSRIRSKYNNYATYYADSVKGRFTISRDDSKNT | |
| | LYLQMNSLRAEDTAVYYCVRASNFPASYVSYFAYWGQGT | |
| | LVTVSS | |
| CD3ₒₚₜ VH | EVQLLESGGGLVQPGGSLRLSCAASGFDFDNYAMNWVRQ | 50 |
| (P035.064) | APGKGLEWVSRIRSKHNGYATYYADSVKGRFTISRDDSKN | |
| | TLYLQMNSLRAEDTAVYYCVRASNFPSSYVSYFGYWGQG | |
| | TLVTVSS | |
| CD3ₒₚₜ VH | EVQLLESGGGLVQPGGSLRLSCAASGFTFSSYAMNWVRQA | 51 |
| (P021.045) | PGKGLEWVSRIRSKYNNYATYYADSVKGRFTISRDDSKNT | |
| | LYLQMNSLRAEDTAVYYCVRASNFPSSYVSYFAYWGQGT | |
| | LVTVSS | |
| CD3ₒₚₜ VH | EVQLLESGGGLVQPGGSLRLSCAASGFQFDNYAMNWVRQ | 52 |
| (P004.042) | APGKGLEWVSRIRTKYNEYATYYADSVKGRFTISRDDSKN | |
| | TLYLQMNSLRAEDTAVYYCVRASNFPQSYVSYFGYWGQG | |
| | TLVTVSS | |
| CD3_{orig}/ | GSSTGAVTTSNYAN | 53 |
| CD3ₒₚₜ | | |
| LCDR1 | | |
| CD3_{orig}/ | GTNKRAP | 54 |
| CD3ₒₚₜ | | |
| LCDR2 | | |
| CD3_{orig}/ | ALWYSNLWV | 55 |
| CD3ₒₚₜ | | |
| LCDR3 | | |
| CD3_{orig}/ | QAVVTQEPSLTVSPGGTVTLTCGSSTGAVTTSNYANWVQE | 56 |
| CD3ₒₚₜ VL | KPGQAFRGLIGGTNKRAPGTPARFSGSLLGGKAALTLSGAQ | |
| | PEDEAEYYCALWYSNLWVFGGGTKLTVL | |
| TYRP1 | DYFLH | 57 |
| HCDR1 | | |
| TYRP1 | WINPDNGNTVYAQKFQG | 58 |
| HCDR2 | | |
| TYRP1 | RDYTYEKAALDY | 59 |
| HCDR3 | | |
| TYRP1 VH | QVQLVQSGAEVKKPGASVKVSCKASGFNIKDYFLHWVRQ | 60 |
| | APGQGLEWMGWINPDNGNTVYAQKFQGRVTMTADTSTST | |
| | VYMELSSLRSEDTAVYYCTRRDYTYEKAALDYWGQGTLV | |
| | TVSS | |
| TYRP1 | RASGNIYNYLA | 61 |
| LCDR1 | | |
| TYRP1 | DAKTLAD | 62 |
| LCDR2 | | |
| TYRP1 | QHFWSLPFT | 63 |
| LCDR3 | | |
| TYRP1 VL | DIQMTQSPSSLSASVGDRVTITCRASGNIYNYLAWYQQKPG | 64 |
| | KVPKLLIYDAKTLADGVPSRFSGSGSGTDFTLTISSLQPEDV | |
| | ATYYCQHFWSLPFTFGQGTKLEIK | |
| TYRP1 VH- | QVQLVQSGAEVKKPGASVKVSCKASGFNIKDYFLHWVRQ | 65 |
| CH1(EE)- | APGQGLEWMGWINPDNGNTVYAQKFQGRVTMTADTSTST | |
| CD3_{orig}/CD3ₒₚₜ | VYMELSSLRSEDTAVYYCTRRDYTYEKAALDYWGQGTLV | |
| VL-CH1- | TVSSASTKGPSVFPLAPSSKSTSGGTAALGCLVEDYFPEPVT | |
| Fc (knob, | VSWNSGALTSGVHTFPAVLQSSGLYSLSSVVTVPSSSLGTQ | |
| PGLALA) | TYICNVNHKPSNTKVDEKVEPKSCDGGGGSGGGGSQAVVT | |
| | QEPSLTVSPGGTVTLTCGSSTGAVTTSNYANWVQEKPGQA | |
| | FRGLIGGTNKRAPGTPARFSGSLLGGKAALTLSGAQPEDEA | |
| | EYYCALWYSNLWVFGGGTKLTVLSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVDK | |
| | KVEPKSCDKTHTCPPCPAPEAAGGPSVFLFPPKPKDTLMISR | |
| | TPEVTCVVVDVSHEDPEVKFNWYVDGVEVHNAKTKPREE | |
| | QYNSTYRVVSVLTVLHQDWLNGKEYKCKVSNKALGAPIE | |
| | KTISKAKGQPREPQVYTLPPCRDELTKNQVSLWCLVKGFY | |
| | PSDIAVEWESNGQPENNYKTTPPVLDSDGSFFLYSKLTVDK | |
| | SRWQQGNVFSCSVMHEALHNHYTQKSLSLSP | |
| TYRP1 VH- | QVQLVQSGAEVKKPGASVKVSCKASGFNIKDYFLHWVRQ | 66 |
| CH1(EE)-Fc | APGQGLEWMGWINPDNGNTVYAQKFQGRVTMTADTSTST | |
| (hole, | VYMELSSLRSEDTAVYYCTRRDYTYEKAALDYWGQGTLV | |
| PGLALA) | TVSSASTKGPSVFPLAPSSKSTSGGTAALGCLVEDYFPEPVT | |
| | VSWNSGALTSGVHTFPAVLQSSGLYSLSSVVTVPSSSLGTQ | |
| | TYICNVNHKPSNTKVDEKVEPKSCDKTHTCPPCPAPEAAG | |
| | GPSVFLFPPKPKDTLMISRTPEVTCVVVDVSHEDPEVKENW | |
| | YVDGVEVHNAKTKPREEQYNSTYRVVSVLTVLHQDWLNG | |
| | KEYKCKVSNKALGAPIEKTISKAKGQPREPQVCTLPPSRDE | |
| | LTKNQVSLSCAVKGFYPSDIAVEWESNGQPENNYKTTPPV | |
| | LDSDGSFFLVSKLTVDKSRWQQGNVFSCSVMHEALHNHY | |
| | TQKSLSLSP | |
| TYRP1 VL- | DIQMTQSPSSLSASVGDRVTITCRASGNIYNYLAWYQQKPG | 67 |
| CL(RK) | KVPKLLIYDAKTLADGVPSRFSGSGSGTDFTLTISSLQPEDV | |
| | ATYYCQHFWSLPFTFGQGTKLEIKRTVAAPSVFIFPPSDRKL | |
| | KSGTASVVCLLNNFYPREAKVQWKVDNALQSGNSQESVT | |
| | EQDSKDSTYSLSSTLTLSKADYEKHKVYACEVTHQGLSSPV | |
| | TKSFNRGEC | |
| CD3_{orig} VH- | EVQLLESGGGLVQPGGSLRLSCAASGFTFSTYAMNWVRQA | 68 |
| CL | PGKGLEWVSRIRSKYNNYATYYADSVKGRFTISRDDSKNT | |
| | LYLQMNSLRAEDTAVYYCVRHGNFGNSYVSWFAYWGQG | |
| | TLVTVSSASVAAPSVFIFPPSDEQLKSGTASVVCLLNNFYPR | |
| | EAKVQWKVDNALQSGNSQESVTEQDSKDSTYSLSSTLTLS | |
| | KADYEKHKVYACEVTHQGLSSPVTKSFNRGEC | |
| CD3ₒₚₜ | EVQLLESGGGLVQPGGSLRLSCAASGFTFESYAMNWVRQA | 69 |
| (P033.078) | PGKGLEWVSRIRSKYNEYATYYADSVKGRFTISRDDSKNT | |
| VH-CL | LYLQMNSLRAEDTAVYYCVRASNFPSSFVSYFGYWGQGTL | |
| | VTVSSASVAAPSVFIFPPSDEQLKSGTASVVCLLNNFYPREA | |
| | KVQWKVDNALQSGNSQESVTEQDSKDSTYSLSSTLTLSKA | |
| | DYEKHKVYACEVTHQGLSSPVTKSFNRGEC | |
| CD3ₒₚₜ | EVQLLESGGGLVQPGGSLRLSCAASGFTFSSYAMNWVRQA | 70 |
| (P035.093) | PGKGLEWVSRIRSKYNNYATYYADSVKGRFTISRDDSKNT | |
| VH-CL | LYLQMNSLRAEDTAVYYCVRASNFPASYVSYFAYWGQGT | |
| | LVTVSSASVAAPSVFIFPPSDEQLKSGTASVVCLLNNFYPRE | |
| | AKVQWKVDNALQSGNSQESVTEQDSKDSTYSLSSTLTLSK | |
| | ADYEKHKVYACEVTHQGLSSPVTKSFNRGEC | |
| CD3ₒₚₜ | EVQLLESGGGLVQPGGSLRLSCAASGFDFDNYAMNWVRQ | 71 |
| (P035.064) | APGKGLEWVSRIRSKHNGYATYYADSVKGRFTISRDDSKN | |
| VH-CL | TLYLQMNSLRAEDTAVYYCVRASNFPSSYVSYFGYWGQG | |
| | TLVTVSSASVAAPSVFIFPPSDEQLKSGTASVVCLLNNFYPR | |
| | EAKVQWKVDNALQSGNSQESVTEQDSKDSTYSLSSTLTLS | |
| | KADYEKHKVYACEVTHQGLSSPVTKSFNRGEC | |
| CD3ₒₚₜ | EVQLLESGGGLVQPGGSLRLSCAASGFTFSSYAMNWVRQA | 72 |
| (P021.045) | PGKGLEWVSRIRSKYNNYATYYADSVKGRFTISRDDSKNT | |
| VH-CL | LYLQMNSLRAEDTAVYYCVRASNFPSSYVSYFAYWGQGT | |
| | LVTVSSASVAAPSVFIFPPSDEQLKSGTASVVCLLNNFYPRE | |
| | AKVQWKVDNALQSGNSQESVTEQDSKDSTYSLSSTLTLSK | |
| | ADYEKHKVYACEVTHQGLSSPVTKSFNRGEC | |
| CD3ₒₚₜ | EVQLLESGGGLVQPGGSLRLSCAASGFQFDNYAMNWVRQ | 73 |
| (P004.042) | APGKGLEWVSRIRTKYNEYATYYADSVKGRFTISRDDSKN | |
| VH-CL | TLYLQMNSLRAEDTAVYYCVRASNFPQSYVSYFGYWGQG | |
| | TLVTVSSASVAAPSVFIFPPSDEQLKSGTASVVCLLNNFYPR | |
| | EAKVQWKVDNALQSGNSQESVTEQDSKDSTYSLSSTLTLS | |
| | KADYEKHKVYACEVTHQGLSSPVTKSFNRGEC | |
| Human CD3 | QDGNEEMGGITQTPYKVSISGTTVILTCPQYPGSEILWQHN | 74 |
| epsilon | DKNIGGDEDDKNIGSDEDHLSLKEFSELEQSGYYVCYPRGS | |
| stalk- Fc | KPEDANFYLYLRARVSENCVDEQLYFQGGSPKSADKTHTC | |
| (knob)-Avi | PPCPAPELLGGPSVFLFPPKPKDTLMISRTPEVTCVVVDVSH | |
| | EDPEVKFNWYVDGVEVHNAKTKPREEQYNSTYRVVSVLT | |
| | VLHQDWLNGKEYKCKVSNKALPAPIEKTISKAKGQPREPQ | |
| | VYTLPPCRDELTKNQVSLWCLVKGFYPSDIAVEWESNGQP | |
| | ENNYKTTPPVLDSDGSFFLYSKLTVDKSRWQQGNVFSCSV | |
| | MHEALHNHYTQKSLSLSPGKSGGLNDIFEAQKIEWHE | |
| Human CD3 | FKIPIEELEDRVFVNCNTSITWVEGTVGTLLSDITRLDLGKRI | 75 |
| delta stalk- | LDPRGIYRCNGTDIYKDKESTVQVHYRMCRSEQLYFQGDK | |
| Fc (hole)- | THTCPPCPAPELLGGPSVFLFPPKPKDTLMISRTPEVTCVVV | |
| Avi | DVSHEDPEVKFNWYVDGVEVHNAKTKPREEQYNSTYRVV | |
| | SVLTVLHQDWLNGKEYKCKVSNKALPAPIEKTISKAKGQP | |
| | REPQVCTLPPSRDELTKNQVSLSCAVKGFYPSDIAVEWESN | |
| | GQPENNYKTTPPVLDSDGSFFLVSKLTVDKSRWQQGNVFS | |
| | CSVMHEALHNHYTQKSLSLSPGKSGGLNDIFEAQKIEWHE | |
| Cynomolgus | QDGNEEMGSITQTPYQVSISGTTVILTCSQHLGSEAQWQHN | 76 |
| CD3 epsilon | GKNKEDSGDRLFLPEFSEMEQSGYYVCYPRGSNPEDASHH | |
| stalk-Fc | LYLKARVSENCVDEQLYFQGGSPKSADKTHTCPPCPAPELL | |
| (knob)-Avi | GGPSVFLFPPKPKDTLMISRTPEVTCVVVDVSHEDPEVKEN | |
| | WYVDGVEVHNAKTKPREEQYNSTYRVVSVLTVLHQDWL | |
| | NGKEYKCKVSNKALPAPIEKTISKAKGQPREPQVYTLPPCR | |
| | DELTKNQVSLWCLVKGFYPSDIAVEWESNGQPENNYKTTP | |
| | PVLDSDGSFFLYSKLTVDKSRWQQGNVFSCSVMHEALHNH | |
| | YTQKSLSLSPGKSGGLNDIFEAQKIEWHE | |
| Cynomolgus | FKIPVEELEDRVFVKCNTSVTWVEGTVGTLLTNNTRLDLG | 77 |
| CD3 delta | KRILDPRGIYRCNGTDIYKDKESAVQVHYRMSQNCVDEQL | |
| stalk-Fc | YFQGGSPKSADKTHTCPPCPAPELLGGPSVFLFPPKPKDTL | |
| (hole)-Avi | MISRTPEVTCVVVDVSHEDPEVKFNWYVDGVEVHNAKTK | |
| | PREEQYNSTYRVVSVLTVLHQDWLNGKEYKCKVSNKALP | |
| | APIEKTISKAKGQPREPQVCTLPPSRDELTKNQVSLSCAVKG | |
| | FYPSDIAVEWESNGQPENNYKTTPPVLDSDGSFFLVSKLTV | |
| | DKSRWQQGNVFSCSVMHEALHNHYTQKSLSLSPGKSGGL | |
| | NDIFEAQKIEWHE | |
| Human CD3 | QDGNEEMGGITQTPYKVSISGTTVILTCPQYPGSEILWQHN | 78 |
| | DKNIGGDEDDKNIGSDEDHLSLKEFSELEQSGYYVCYPRGS | |
| | KPEDANFYLYLRARVCENCMEMDVMSVATIVIVDICITGG | |
| | LLLLVYYWSKNRKAKAKPVTRGAGAGGRQRGQNKERPPP | |
| | VPNPDYEPIRKGQRDLYSGLNQRRI | |
| Cynomolgus | QDGNEEMGSITQTPYQVSISGTTVILTCSQHLGSEAQWQHN | 79 |
| CD3 | GKNKEDSGDRLFLPEFSEMEQSGYYVCYPRGSNPEDASHH | |
| | LYLKARVCENCMEMDVMAVATIVIVDICITLGLLLLVYYW | |
| | SKNRKAKAKPVTRGAGAGGRQRGQNKERPPPVPNPDYEPI | |
| | RKGQQDLYSGLNQRRI | |
| hIgG1 Fc | DKTHTCPPCPAPELLGGPSVFLFPPKPKDTLMISRTPEVTCV | 80 |
| region | VVDVSHEDPEVKFNWYVDGVEVHNAKTKPREEQYNSTYR | |
| | VVSVLTVLHQDWLNGKEYKCKVSNKALPAPIEKTISKAKG | |
| | QPREPQVYTLPPSRDELTKNQVSLTCLVKGFYPSDIAVEWE | |
| | SNGQPENNYKTTPPVLDSDGSFFLYSKLTVDKSRWQQGNV | |
| | FSCSVMHEALHNHYTQKSLSLSP | |
| linker | GGGGSGGGGS | 81 |
| linker | DGGGGSGGGGS | 82 |
| Human | RTVAAPSVFIFPPSDEQLKSGTASVVCLLNNFYPREAKVQW | 83 |
| kappa CL | KVDNALQSGNSQESVTEQDSKDSTYSLSSTLTLSKADYEK | |
| domain | HKVYACEVTHQGLSSPVTKSFNRGEC | |
| Human | QPKAAPSVTLFPPSSEELQANKATLVCLISDFYPGAVTVAW | 84 |
| lambda CL | KADSSPVKAGVETTTPSKQSNNKYAASSYLSLTPEQWKSH | |
| domain | RSYSCQVTHEGSTVEKTVAPTECS | |
| Human IgG1 | ASTKGPSVFPLAPSSKSTSGGTAALGCLVKDYFPEPVTVSW | 85 |
| heavy chain | NSGALTSGVHTFPAVLQSSGLYSLSSVVTVPSSSLGTQTYIC | |
| constant | NVNHKPSNTKVDKKVEPKSCDKTHTCPPCPAPELLGGPSVF | |
| region (CH1- | LFPPKPKDTLMISRTPEVTCVVVDVSHEDPEVKFNWYVDG | |
| CH2-CH3) | VEVHNAKTKPREEQYNSTYRVVSVLTVLHQDWLNGKEYK | |
| | CKVSNKALPAPIEKTISKAKGQPREPQVYTLPPSRDELTKN | |
| | QVSLTCLVKGFYPSDIAVEWESNGQPENNYKTTPPVLDSD | |
| | GSFFLYSKLTVDKSRWQQGNVFSCSVMHEALHNHYTQKS | |
| | LSLSP | |

Exemplary immune activating Fc binding molecules capable of specific
binding to the activating T cell antigen CD3
Anti-P329G (M-1.7.24) x anti-CD3 (CH2527) 2 + 1 TCB (lambda LC)

| | | |
|---|---|---|
| LC1 | QAVVTQESALTTSPGETVTLTCRSSTGAVTTSNYANWVQE | 86 |
| PG | KPDHLFTGLIGGTNKRAPGVPARFSGSLIGDKAALTITGAQ | |
| | TEDEAIYFCALWYSNHWVFGGGTKLTVLGQPKAAPSVTLF | |
| | PPSSKKLQANKATLVCLISDFYPGAVTVAWKADSSPVKAG | |
| | VETTTPSKQSNNKYAASSYLSLTPEQWKSHRSYSCQVTHE | |
| | GSTVEKTVAPTECS | |
| LC2 | EVQLLESGGGLVQPGGSLRLSCAASGFTFSTYAMNWVRQA | 68 |
| CD3 | PGKGLEWVSRIRSKYNNYATYYADSVKGRFTISRDDSKNT | |
| (crossed) | LYLQMNSLRAEDTAVYYCVRHGNFGNSYVSWFAYWGQG | |
| | TLVTVSSASVAAPSVFIFPPSDEQLKSGTASVVCLLNNFYPR | |
| | EAKVQWKVDNALQSGNSQESVTEQDSKDSTYSLSSTLTLS | |
| | KADYEKHKVYACEVTHQGLSSPVTKSFNRGEC | |
| HC1 | EVKLLESGGGLVQPGGSLKLSCAASGFDFSRYWMNWVRQ | 87 |
| | APGKGLEWIGEITPDSSTINYTPSLKDKFIISRDNAKNTLYLQ | |
| | MIKVRSEDTALYYCVRPYDYGAWFASWGQGTLVTVSAAS | |
| | TKGPSVFPLAPSSKSTSGGTAALGCLVEDYFPEPVTVSWNS | |
| | GALTSGVHTFPAVLQSSGLYSLSSVVTVPSSSLGTQTYICNV | |
| | NHKPSNTKVDEKVEPKSCDGGGGSGGGGSQAVVTQEPSLT | |
| | VSPGGTVTLTCGSSTGAVTTSNYANWVQEKPGQAFRGLIG | |
| | GTNKRAPGTPARFSGSLLGGKAALTLSGAQPEDEAEYYCA | |
| PG | LWYSNLWVFGGGTKLTVLSSASTKGPSVFPLAPSSKSTSGG | |
| CD3 | TAALGCLVKDYFPEPVTVSWNSGALTSGVHTFPAVLQSSG | |
| knob | LYSLSSVVTVPSSSLGTQTYICNVNHKPSNTKVDKKVEPKS | |
| | CDKTHTCPPCPAPEAAGGPSVFLFPPKPKDTLMISRTPEVTC | |
| | VVVDVSHEDPEVKFNWYVDGVEVHNAKTKPREEQYNSTY | |
| | RVVSVLTVLHQDWLNGKEYKCKVSNKALPAPIEKTISKAK | |
| | GQPREPQVYTLPPCRDELTKNQVSLWCLVKGFYPSDIAVE | |
| | WESNGQPENNYKTTPPVLDSDGSFFLYSKLTVDKSRWQQG | |
| | NVFSCSVMHEALHNHYTQKSLSLSP | |
| HC2 | EVKLLESGGGLVQPGGSLKLSCAASGFDFSRYWMNWVRQ | 88 |
| PG | APGKGLEWIGEITPDSSTINYTPSLKDKFIISRDNAKNTLYLQ | |
| hole | MIKVRSEDTALYYCVRPYDYGAWFASWGQGTLVTVSAAS | |
| | TKGPSVFPLAPSSKSTSGGTAALGCLVEDYFPEPVTVSWNS | |
| | GALTSGVHTFPAVLQSSGLYSLSSVVTVPSSSLGTQTYICNV | |
| | NHKPSNTKVDEKVEPKSCDKTHTCPPCPAPEAAGGPSVFLF | |
| | PPKPKDTLMISRTPEVTCVVVDVSHEDPEVKFNWYVDGVE | |
| | VHNAKTKPREEQYNSTYRVVSVLTVLHQDWLNGKEYKCK | |
| | VSNKALPAPIEKTISKAKGQPREPQVCTLPPSRDELTKNQVS | |
| | LSCAVKGFYPSDIAVEWESNGQPENNYKTTPPVLDSDGSFF | |
| | LVSKLTVDKSRWQQGNVFSCSVMHEALHNHYTQKSLSLSP | |

Anti-P329G (VH3VL1) x anti-CD3 (CH2527) 1 + 1 TCB

| | | |
|---|---|---|
| LC1 | QAVVTQEPSLTVSPGGTVTLTCRSSTGAVTTSNYANWVQE | 89 |
| PG | KPDHLFTGLIGGTNKRAPGTPARFSGSLLGGKAALTLSGAQ | |
| | PEDEAEYYCALWYSNHWVFGGGTKLTVLGQPKAAPSVTL | |
| | FPPSSKKLQANKATLVCLISDFYPGAVTVAWKADSSPVKA | |
| | GVETTTPSKQSNNKYAASSYLSLTPEQWKSHRSYSCQVTH | |
| | EGSTVEKTVAPTECS | |
| LC2 | EVQLLESGGGLVQPGGSLRLSCAASGFTFSTYAMNWVRQA | 68 |
| CD3 | PGKGLEWVSRIRSKYNNYATYYADSVKGRFTISRDDSKNT | |
| (crossed) | LYLQMNSLRAEDTAVYYCVRHGNFGNSYVSWFAYWGQG | |
| | TLVTVSSASVAAPSVFIFPPSDEQLKSGTASVVCLLNNFYPR | |
| | EAKVQWKVDNALQSGNSQESVTEQDSKDSTYSLSSTLTLS | |
| | KADYEKHKVYACEVTHQGLSSPVTKSFNRGEC | |
| HC1 | EVQLVESGGGLVQPGGSLRLSCAASGFTFSRYWMNWVRQ | 90 |
| PG | APGKGLEWVGEITPDSSTINYAPSLKGRFTISRDNAKNSLYL | |
| hole | QMNSLRAEDTAVYYCARPYDYGAWFASWGQGTLVTVSS | |
| | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDKTHTCPPCPAPEAAGGPSV | |
| | FLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALPAPIEKTISKAKGQPREPQVCTLPPSRDELTKN | |
| | QVSLSCAVKGFYPSDIAVEWESNGQPENNYKTTPPVLDSD | |
| | GSFFLVSKLTVDKSRWQQGNVFSCSVMHEALHNHYTQKS | |
| | LSLSP | |
| HC2 | QAVVTQEPSLTVSPGGTVTLTCGSSTGAVTTSNYANWVQE | 91 |
| CD3 | KPGQAFRGLIGGTNKRAPGTPARFSGSLLGGKAALTLSGAQ | |
| knob | PEDEAEYYCALWYSNLWVFGGGTKLTVLSSASTKGPSVFP | |
| | LAPSSKSTSGGTAALGCLVKDYFPEPVTVSWNSGALTSGV | |
| | HTFPAVLQSSGLYSLSSVVTVPSSSLGTQTYICNVNHKPSNT | |
| | KVDKKVEPKSCDKTHTCPPCPAPEAAGGPSVFLFPPKPKDT | |
| | LMISRTPEVTCVVVDVSHEDPEVKFNWYVDGVEVHNAKT | |
| | KPREEQYNSTYRVVSVLTVLHQDWLNGKEYKCKVSNKAL | |
| | PAPIEKTISKAKGQPREPQVYTLPPCRDELTKNQVSLWCLV | |
| | KGFYPSDIAVEWESNGQPENNYKTTPPVLDSDGSFFLYSKL | |
| | TVDKSRWQQGNVFSCSVMHEALHNHYTQKSLSLSP | |

Anti-P329G (VH3VL1) x anti-CD3 (P035.093) 1 + 1 TCB

| | | |
|---|---|---|
| LC1 | QAVVTQEPSLTVSPGGTVTLTCRSSTGAVTTSNYANWVQE | 89 |
| PG | KPDHLFTGLIGGTNKRAPGTPARFSGSLLGGKAALTLSGAQ | |
| | PEDEAEYYCALWYSNHWVFGGGTKLTVLGQPKAAPSVTL | |
| | FPPSSKKLQANKATLVCLISDFYPGAVTVAWKADSSPVKA | |
| | GVETTTPSKQSNNKYAASSYLSLTPEQWKSHRSYSCQVTH | |
| | EGSTVEKTVAPTECS | |
| LC2 | EVQLLESGGGLVQPGGSLRLSCAASGFTFSSYAMNWVRQA | 70 |
| CD3 | PGKGLEWVSRIRSKYNNYATYYADSVKGRFTISRDDSKNT | |
| (crossed) | LYLQMNSLRAEDTAVYYCVRASNFPASYVSYFAYWGQGT | |
| | LVTVSSASVAAPSVFIFPPSDEQLKSGTASVVCLLNNFYPRE | |
| | AKVQWKVDNALQSGNSQESVTEQDSKDSTYSLSSTLTLSK | |
| | ADYEKHKVYACEVTHQGLSSPVTKSFNRGEC | |
| HC1 | EVQLVESGGGLVQPGGSLRLSCAASGFTFSRYWMNWVRQ | 90 |
| PG | APGKGLEWVGEITPDSSTINYAPSLKGRFTISRDNAKNSLYL | |
| hole | QMNSLRAEDTAVYYCARPYDYGAWFASWGQGTLVTVSS | |
| | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDKTHTCPPCPAPEAAGGPSV | |
| | FLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALPAPIEKTISKAKGQPREPQVCTLPPSRDELTKN | |
| | QVSLSCAVKGFYPSDIAVEWESNGQPENNYKTTPPVLDSD | |
| | GSFFLVSKLTVDKSRWQQGNVFSCSVMHEALHNHYTQKS | |
| | LSLSP | |
| HC2 | QAVVTQEPSLTVSPGGTVTLTCGSSTGAVTTSNYANWVQE | 91 |
| CD3 | KPGQAFRGLIGGTNKRAPGTPARFSGSLLGGKAALTLSGAQ | |
| knob | PEDEAEYYCALWYSNLWVFGGGTKLTVLSSASTKGPSVFP | |
| | LAPSSKSTSGGTAALGCLVKDYFPEPVTVSWNSGALTSGV | |
| | HTFPAVLQSSGLYSLSSVVTVPSSSLGTQTYICNVNHKPSNT | |
| | KVDKKVEPKSCDKTHTCPPCPAPEAAGGPSVFLFPPKPKDT | |
| | LMISRTPEVTCVVVDVSHEDPEVKFNWYVDGVEVHNAKT | |
| | KPREEQYNSTYRVVSVLTVLHQDWLNGKEYKCKVSNKAL | |
| | PAPIEKTISKAKGQPREPQVYTLPPCRDELTKNQVSLWCLV | |
| | KGFYPSDIAVEWESNGQPENNYKTTPPVLDSDGSFFLYSKL | |
| | TVDKSRWQQGNVFSCSVMHEALHNHYTQKSLSLSP | |

Anti-P329G (VH3VL1) x anti-CD3 (P035.093) 2 + 1 TCB

| | | |
|---|---|---|
| LC1 | QAVVTQEPSLTVSPGGTVTLTCRSSTGAVTTSNYANWVQE | 89 |
| PG | KPDHLFTGLIGGTNKRAPGTPARFSGSLLGGKAALTLSGAQ | |
| | PEDEAEYYCALWYSNHWVFGGGTKLTVLGQPKAAPSVTL | |
| | FPPSSKKLQANKATLVCLISDFYPGAVTVAWKADSSPVKA | |
| | GVETTTPSKQSNNKYAASSYLSLTPEQWKSHRSYSCQVTH | |
| | EGSTVEKTVAPTECS | |
| LC2 | EVQLLESGGGLVQPGGSLRLSCAASGFTFSSYAMNWVRQA | 70 |
| CD3 | PGKGLEWVSRIRSKYNNYATYYADSVKGRFTISRDDSKNT | |
| (crossed) | LYLQMNSLRAEDTAVYYCVRASNFPASYVSYFAYWGQGT | |
| | LVTVSSASVAAPSVFIFPPSDEQLKSGTASVVCLLNNFYPRE | |
| | AKVQWKVDNALQSGNSQESVTEQDSKDSTYSLSSTLTLSK | |
| | ADYEKHKVYACEVTHQGLSSPVTKSFNRGEC | |
| HC1 | EVQLVESGGGLVQPGGSLRLSCAASGFTFSRYWMNWVRQ | 90 |
| PG | APGKGLEWVGEITPDSSTINYAPSLKGRFTISRDNAKNSLYL | |
| hole | QMNSLRAEDTAVYYCARPYDYGAWFASWGQGTLVTVSS | |
| | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDKTHTCPPCPAPEAAGGPSV | |
| | FLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALPAPIEKTISKAKGQPREPQVCTLPPSRDELTKN | |
| | QVSLSCAVKGFYPSDIAVEWESNGQPENNYKTTPPVLDSD | |
| | GSFFLVSKLTVDKSRWQQGNVFSCSVMHEALHNHYTQKS | |
| | LSLSP | |
| HC2 | EVQLVESGGGLVQPGGSLRLSCAASGFTFSRYWMNWVRQ | 92 |
| PG | APGKGLEWVGEITPDSSTINYAPSLKGRFTISRDNAKNSLYL | |
| CD3 | QMNSLRAEDTAVYYCARPYDYGAWFASWGQGTLVTVSS | |
| knob | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDGGGGSGGGGGQAVVTQEP | |
| | SLTVSPGGTVTLTCGSSTGAVTTSNYANWVQEKPGQAFRG | |
| | LIGGTNKRAPGTPARFSGSLLGGKAALTLSGAQPEDEAEYY | |
| | CALWYSNLWVFGGGTKLTVLSSASTKGPSVFPLAPSSKSTS | |
| | GGTAALGCLVKDYFPEPVTVSWNSGALTSGVHTFPAVLQS | |
| | SGLYSLSSVVTVPSSSLGTQTYICNVNHKPSNTKVDKKVEP | |
| | KSCDKTHTCPPCPAPEAAGGPSVFLFPPKPKDTLMISRTPEV | |
| | TCVVVDVSHEDPEVKFNWYVDGVEVHNAKTKPREEQYNS | |
| | TYRVVSVLTVLHQDWLNGKEYKCKVSNKALPAPIEKTISK | |
| | AKGQPREPQVYTLPPCRDELTKNQVSLWCLVKGFYPSDIA | |
| | VEWESNGQPENNYKTTPPVLDSDGSFFLYSKLTVDKSRWQ | |
| | QGNVFSCSVMHEALHNHYTQKSLSLSP | |

Anti-P329G (M-1.7.24) x anti-CD3 (CH2527) 2 + 1 TCB (kappa LC)

| | | |
|---|---|---|
| LC1 | QAVVTQESALTTSPGETVTLTCRSSTGAVTTSNYANWVQE | 93 |
| PG | KPDHLFTGLIGGTNKRAPGVPARFSGSLIGDKAALTITGAQ | |
| | TEDEAIYFCALWYSNHWVFGGGTKLTVLRTVAAPSVFIFPP | |
| | SDRKLKSGTASVVCLLNNFYPREAKVQWKVDNALQSGNS | |
| | QESVTEQDSKDSTYSLSSTLTLSKADYEKHKVYACEVTHQ | |
| | GLSSPVTKSFNRGEC | |
| LC2 | EVQLLESGGGLVQPGGSLRLSCAASGFTFSTYAMNWVRQA | 68 |
| CD3 | PGKGLEWVSRIRSKYNNYATYYADSVKGRFTISRDDSKNT | |
| (crossed) | LYLQMNSLRAEDTAVYYCVRHGNFGNSYVSWFAYWGQG | |
| | TLVTVSSASVAAPSVFIFPPSDEQLKSGTASVVCLLNNFYPR | |
| | EAKVQWKVDNALQSGNSQESVTEQDSKDSTYSLSSTLTLS | |
| | KADYEKHKVYACEVTHQGLSSPVTKSFNRGEC | |
| HC1 | EVKLLESGGGLVQPGGSLKLSCAASGFDFSRYWMNWVRQ | 87 |
| PG | APGKGLEWIGEITPDSSTINYTPSLKDKFIISRDNAKNTLYLQ | |
| CD3 | MIKVRSEDTALYYCVRPYDYGAWFASWGQGTLVTVSAAS | |
| knob | TKGPSVFPLAPSSKSTSGGTAALGCLVEDYFPEPVTVSWNS | |
| | GALTSGVHTFPAVLQSSGLYSLSSVVTVPSSSLGTQTYICNV | |
| | NHKPSNTKVDEKVEPKSCDGGGGSGGGGSQAVVTQEPSLT | |
| | VSPGGTVTLTCGSSTGAVTTSNYANWVQEKPGQAFRGLIG | |
| | GTNKRAPGTPARFSGSLLGGKAALTLSGAQPEDEAEYYCA | |
| | LWYSNLWVFGGGTKLTVLSSASTKGPSVFPLAPSSKSTSGG | |
| | TAALGCLVKDYFPEPVTVSWNSGALTSGVHTFPAVLQSSG | |
| | LYSLSSVVTVPSSSLGTQTYICNVNHKPSNTKVDKKVEPKS | |
| | CDKTHTCPPCPAPEAAGGPSVFLFPPKPKDTLMISRTPEVTC | |
| | VVVDVSHEDPEVKFNWYVDGVEVHNAKTKPREEQYNSTY | |
| | RVVSVLTVLHQDWLNGKEYKCKVSNKALPAPIEKTISKAK | |
| | GQPREPQVYTLPPCRDELTKNQVSLWCLVKGFYPSDIAVE | |
| | WESNGQPENNYKTTPPVLDSDGSFFLYSKLTVDKSRWQQG | |
| | NVFSCSVMHEALHNHYTQKSLSLSP | |
| HC2 | EVKLLESGGGLVQPGGSLKLSCAASGFDFSRYWMNWVRQ | 88 |
| PG | APGKGLEWIGEITPDSSTINYTPSLKDKFIISRDNAKNTLYLQ | |
| hole | MIKVRSEDTALYYCVRPYDYGAWFASWGQGTLVTVSAAS | |
| | TKGPSVFPLAPSSKSTSGGTAALGCLVEDYFPEPVTVSWNS | |
| | GALTSGVHTFPAVLQSSGLYSLSSVVTVPSSSLGTQTYICNV | |
| | NHKPSNTKVDEKVEPKSCDKTHTCPPCPAPEAAGGPSVFLF | |
| | PPKPKDTLMISRTPEVTCVVVDVSHEDPEVKFNWYVDGVE | |
| | VHNAKTKPREEQYNSTYRVVSVLTVLHQDWLNGKEYKCK | |
| | VSNKALPAPIEKTISKAKGQPREPQVCTLPPSRDELTKNQVS | |
| | LSCAVKGFYPSDIAVEWESNGQPENNYKTTPPVLDSDGSFF | |
| | LVSKLTVDKSRWQQGNVFSCSVMHEALHNHYTQKSLSLSP | |

Exemplary immune activating Fc binding molecules capable of specific
binding to the costimulatory T cell antigen CD28
anti-CD28 binder variant 15

| | | |
|---|---|---|
| HCDR1 | SYYIH | 94 |
| HCDR2 | SIYPGNVQTNYNEKFKD | 95 |
| HCDR3 | SHYGLDWNFDV | 96 |
| LCDR1 | HASQNIYVFLN | 97 |
| LCDR2 | KASNLHT | 98 |
| LCDR3 | QQGQTYPYT | 99 |
| VH | QVQLVQSGAEVKKPGASVKVSCKASGYTFTSYYIHWVRQ | 100 |
| | APGQGLEWIGSIYPGNVQTNYNEKFKDRATLTVDTSISTAY | |
| | MELSRLRSDDTAVYFCTRSHYGLDWNFDVWGQGTTVTVS | |
| | S | |
| VL | DIQMTQSPSSLSASVGDRVTITCHASQNIYVFLNWYQQKPG | 101 |
| | KAPKLLIYKASNLHTGVPSRFSGSGSGTDFTLTISSLQPEDF | |
| | ATYYCQQGQTYPYTFGGGTKVEIK | |

anti-CD28 binder variant 8

| | | |
|---|---|---|
| HCDR1 | SYYIH | 94 |
| HCDR2 | SIYPGNVQTNYNEKFKD | 95 |
| HCDR3 | SHYGLDFNFDV | 102 |
| LCDR1 | HASQNIYVYLN | 103 |
| LCDR2 | KASNLHT | 98 |
| LCDR3 | QQGQTYPYT | 99 |
| VH | QVQLVQSGAEVKKPGASVKVSCKASGYTFTSYYIHWVRQ | 104 |
| | APGQGLEWIGSIYPGNVQTNYNEKFKDRATLTVDTSISTAY | |
| | MELSRLRSDDTAVYFCTRSHYGLDFNFDVWGQGTTVTVSS | |
| VL | DIQMTQSPSSLSASVGDRVTITCHASQNIYVYLNWYQQKPG | 105 |
| | KAPKLLIYKASNLHTGVPSRFSGSGSGTDFTLTISSLQPEDF | |
| | ATYYCQQGQTYPYTFGGGTKVEIK | |

Anti-P329G (M-1.7.24) x anti-CD28 (TGN1412_variant 15_crossed) 1 + 1

| | | |
|---|---|---|
| LC1 | QAVVTQESALTTSPGETVTLTCRSSTGAVTTSNYANWVQE | 93 |
| PG | KPDHLFTGLIGGTNKRAPGVPARFSGSLIGDKAALTITGAQ | |
| | TEDEAIYFCALWYSNHWVFGGGTKLTVLRTVAAPSVFIFPP | |
| | SDRKLKSGTASVVCLLNNFYPREAKVQWKVDNALQSGNS | |
| | QESVTEQDSKDSTYSLSSTLTLSKADYEKHKVYACEVTHQ | |
| | GLSSPVTKSFNRGEC | |
| LC2 | QVQLVQSGAEVKKPGASVKVSCKASGYTFTSYYIHWVRQ | 106 |
| CD28 | APGQGLEWIGSIYPGNVQTNYNEKFKDRATLTVDTSISTAY | |
| | MELSRLRSDDTAVYFCTRSHYGLDWNFDVWGQGTTVTVS | |
| | SASVAAPSVFIFPPSDEQLKSGTASVVCLLNNFYPREAKVQ | |
| | WKVDNALQSGNSQESVTEQDSKDSTYSLSSTLTLSKADYE | |
| | KHKVYACEVTHQGLSSPVTKSFNRGEC | |
| HC1 | EVKLLESGGGLVQPGGSLKLSCAASGFDFSRYWMNWVRQ | 88 |
| PG | APGKGLEWIGEITPDSSTINYTPSLKDKFIISRDNAKNTLYLQ | |
| hole | MIKVRSEDTALYYCVRPYDYGAWFASWGQGTLVTVSAAS | |
| | TKGPSVFPLAPSSKSTSGGTAALGCLVEDYFPEPVTVSWNS | |
| | GALTSGVHTFPAVLQSSGLYSLSSVVTVPSSSLGTQTYICNV | |
| | NHKPSNTKVDEKVEPKSCDKTHTCPPCPAPEAAGGPSVFLF | |
| | PPKPKDTLMISRTPEVTCVVVDVSHEDPEVKFNWYVDGVE | |
| | VHNAKTKPREEQYNSTYRVVSVLTVLHQDWLNGKEYKCK | |
| | VSNKALPAPIEKTISKAKGQPREPQVCTLPPSRDELTKNQVS | |
| | LSCAVKGFYPSDIAVEWESNGQPENNYKTTPPVLDSDGSFF | |
| | LVSKLTVDKSRWQQGNVFSCSVMHEALHNHYTQKSLSLSP | |
| HC2 knob | DIQMTQSPSSLSASVGDRVTITCHASQNIYVFLNWYQQKPG | 107 |
| | KAPKLLIYKASNLHTGVPSRFSGSGSGTDFTLTISSLQPEDF | |
| | ATYYCQQGQTYPYTFGGGTKVEIKSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVDK | |
| | KVEPKSCDKTHTCPPCPAPEAAGGPSVFLFPPKPKDTLMISR | |
| | TPEVTCVVVDVSHEDPEVKFNWYVDGVEVHNAKTKPREE | |
| | QYNSTYRVVSVLTVLHQDWLNGKEYKCKVSNKALPAPIE | |
| | KTISKAKGQPREPQVYTLPPCRDELTKNQVSLWCLVKGFY | |
| | PSDIAVEWESNGQPENNYKTTPPVLDSDGSFFLYSKLTVDK | |
| | SRWQQGNVFSCSVMHEALHNHYTQKSLSLSP | |

Anti-P329G (M-1.7.24) x anti-CD28 (TGN1412_variant 8_crossed) 1 + 1

| | | |
|---|---|---|
| LC1 | QAVVTQESALTTSPGETVTLTCRSSTGAVTTSNYANWVQE | 93 |
| PG | KPDHLFTGLIGGTNKRAPGVPARFSGSLIGDKAALTITGAQ | |
| | TEDEAIYFCALWYSNHWVFGGGTKLTVLRTVAAPSVFIFPP | |
| | SDRKLKSGTASVVCLLNNFYPREAKVQWKVDNALQSGNS | |
| | QESVTEQDSKDSTYSLSSTLTLSKADYEKHKVYACEVTHQ | |
| | GLSSPVTKSFNRGEC | |
| LC2 | QVQLVQSGAEVKKPGASVKVSCKASGYTFTSYYIHWVRQ | 108 |
| CD28 | APGQGLEWIGSIYPGNVQTNYNEKFKDRATLTVDTSISTAY | |
| | MELSRLRSDDTAVYFCTRSHYGLDFNFDVWGQGTTVTVSS | |
| | ASVAAPSVFIFPPSDEQLKSGTASVVCLLNNFYPREAKVQW | |
| | KVDNALQSGNSQESVTEQDSKDSTYSLSSTLTLSKADYEK | |
| | HKVYACEVTHQGLSSPVTKSFNRGEC | |
| HC1 | EVKLLESGGGLVQPGGSLKLSCAASGFDFSRYWMNWVRQ | 88 |
| PG | APGKGLEWIGEITPDSSTINYTPSLKDKFIISRDNAKNTLYLQ | |
| hole | MIKVRSEDTALYYCVRPYDYGAWFASWGQGTLVTVSAAS | |
| | TKGPSVFPLAPSSKSTSGGTAALGCLVEDYFPEPVTVSWNS | |
| | GALTSGVHTFPAVLQSSGLYSLSSVVTVPSSSLGTQTYICNV | |
| | NHKPSNTKVDEKVEPKSCDKTHTCPPCPAPEAAGGPSVFLF | |
| | PPKPKDTLMISRTPEVTCVVVDVSHEDPEVKFNWYVDGVE | |
| | VHNAKTKPREEQYNSTYRVVSVLTVLHQDWLNGKEYKCK | |
| | VSNKALPAPIEKTISKAKGQPREPQVCTLPPSRDELTKNQVS | |
| | LSCAVKGFYPSDIAVEWESNGQPENNYKTTPPVLDSDGSFF | |
| | LVSKLTVDKSRWQQGNVFSCSVMHEALHNHYTQKSLSLSP | |
| HC2 | DIQMTQSPSSLSASVGDRVTITCHASQNIYVYLNWYQQKPG | 109 |
| CD28 | KAPKLLIYKASNLHTGVPSRFSGSGSGTDFTLTISSLQPEDF | |
| knob | ATYYCQQGQTYPYTFGGGTKVEIKSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVDK | |
| | KVEPKSCDKTHTCPPCPAPEAAGGPSVFLFPPKPKDTLMISR | |
| | TPEVTCVVVDVSHEDPEVKFNWYVDGVEVHNAKTKPREE | |
| | QYNSTYRVVSVLTVLHQDWLNGKEYKCKVSNKALPAPIE | |
| | KTISKAKGQPREPQVYTLPPCRDELTKNQVSLWCLVKGFY | |
| | PSDIAVEWESNGQPENNYKTTPPVLDSDGSFFLYSKLTVDK | |
| | SRWQQGNVFSCSVMHEALHNHYTQKSLSLSP | |

Anti-P329G (VH3VL1) x anti-CD28 (TGN1412_variant 8_crossed) 1 + 1

| | | |
|---|---|---|
| LC1 | QAVVTQEPSLTVSPGGTVTLTCRSSTGAVTTSNYANWVQE | 89 |
| PG | KPDHLFTGLIGGTNKRAPGTPARFSGSLLGGKAALTLSGAQ | |
| | PEDEAEYYCALWYSNHWVFGGGTKLTVLGQPKAAPSVTL | |
| | FPPSSKKLQANKATLVCLISDFYPGAVTVAWKADSSPVKA | |
| | GVETTTPSKQSNNKYAASSYLSLTPEQWKSHRSYSCQVTH | |
| | EGSTVEKTVAPTECS | |
| LC2 | QVQLVQSGAEVKKPGASVKVSCKASGYTFTSYYIHWVRQ | 108 |
| CD28 | APGQGLEWIGSIYPGNVQTNYNEKFKDRATLTVDTSISTAY | |
| | MELSRLRSDDTAVYFCTRSHYGLDFNFDVWGQGTTVTVSS | |
| | ASVAAPSVFIFPPSDEQLKSGTASVVCLLNNFYPREAKVQW | |
| | KVDNALQSGNSQESVTEQDSKDSTYSLSSTLTLSKADYEK | |
| | HKVYACEVTHQGLSSPVTKSFNRGEC | |
| HC1 | EVQLVESGGGLVQPGGSLRLSCAASGFTFSRYWMNWVRQ | 90 |
| PG | APGKGLEWVGEITPDSSTINYAPSLKGRFTISRDNAKNSLYL | |
| hole | QMNSLRAEDTAVYYCARPYDYGAWFASWGQGTLVTVSS | |
| | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDKTHTCPPCPAPEAAGGPSV | |
| | FLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALPAPIEKTISKAKGQPREPQVCTLPPSRDELTKN | |
| | QVSLSCAVKGFYPSDIAVEWESNGQPENNYKTTPPVLDSD | |
| | GSFFLVSKLTVDKSRWQQGNVFSCSVMHEALHNHYTQKS | |
| | LSLSP | |
| HC2 | DIQMTQSPSSLSASVGDRVTITCHASQNIYVYLNWYQQKPG | 109 |
| CD28 | KAPKLLIYKASNLHTGVPSRFSGSGSGTDFTLTISSLQPEDF | |
| knob | ATYYCQQGQTYPYTFGGGTKVEIKSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVDK | |
| | KVEPKSCDKTHTCPPCPAPEAAGGPSVFLFPPKPKDTLMISR | |
| | TPEVTCVVVDVSHEDPEVKFNWYVDGVEVHNAKTKPREE | |
| | QYNSTYRVVSVLTVLHQDWLNGKEYKCKVSNKALPAPIE | |
| | KTISKAKGQPREPQVYTLPPCRDELTKNQVSLWCLVKGFY | |
| | PSDIAVEWESNGQPENNYKTTPPVLDSDGSFFLYSKLTVDK | |
| | SRWQQGNVFSCSVMHEALHNHYTQKSLSLSP | |

Anti-P329G (VH3VL1) x anti-CD28 (TGN1412_variant 8) 1 + 1

| | | |
|---|---|---|
| LC1 | EVQLVESGGGLVQPGGSLRLSCAASGFTFSRYWMNWVRQ | 110 |
| PG | APGKGLEWVGEITPDSSTINYAPSLKGRFTISRDNAKNSLYL | |
| crossed | QMNSLRAEDTAVYYCARPYDYGAWFASWGQGTLVTVSS | |
| | ASVAAPSVFIFPPSDEQLKSGTASVVCLLNNFYPREAKVQW | |
| | KVDNALQSGNSQESVTEQDSKDSTYSLSSTLTLSKADYEK | |
| | HKVYACEVTHQGLSSPVTKSFNRGEC | |
| LC2 | DIQMTQSPSSLSASVGDRVTITCHASQNIYVYLNWYQQKPG | 111 |
| CD28 | KAPKLLIYKASNLHTGVPSRFSGSGSGTDFTLTISSLQPEDF | |
| var 8 | ATYYCQQGQTYPYTFGGGTKVEIKRTVAAPSVFIFPPSDRK | |
| pCON3753 | LKSGTASVVCLLNNFYPREAKVQWKVDNALQSGNSQESV | |
| | TEQDSKDSTYSLSSTLTLSKADYEKHKVYACEVTHQGLSSP | |
| | VTKSFNRGEC | |
| HC1 | QAVVTQEPSLTVSPGGTVTLTCRSSTGAVTTSNYANWVQE | 112 |
| PG | KPDHLFTGLIGGTNKRAPGTPARFSGSLLGGKAALTLSGAQ | |
| | PEDEAEYYCALWYSNHWVFGGGTKLTVLSSASTKGPSVFP | |
| | LAPSSKSTSGGTAALGCLVKDYFPEPVTVSWNSGALTSGV | |
| | HTFPAVLQSSGLYSLSSVVTVPSSSLGTQTYICNVNHKPSNT | |
| | KVDKKVEPKSCDKTHTCPPCPAPEAAGGPSVFLFPPKPKDT | |
| | LMISRTPEVTCVVVDVSHEDPEVKFNWYVDGVEVHNAKT | |
| | KPREEQYNSTYRVVSVLTVLHQDWLNGKEYKCKVSNKAL | |
| | PAPIEKTISKAKGQPREPQVCTLPPSRDELTKNQVSLSCAVK | |
| | GFYPSDIAVEWESNGQPENNYKTTPPVLDSDGSFFLVSKLT | |
| | VDKSRWQQGNVFSCSVMHEALHNHYTQKSLSLSP | |
| HC2 | QVQLVQSGAEVKKPGASVKVSCKASGYTFTSYYIHWVRQ | 113 |
| CD28 | APGQGLEWIGSIYPGNVQTNYNEKFKDRATLTVDTSISTAY | |
| | MELSRLRSDDTAVYFCTRSHYGLDFNFDVWGQGTTVTVSS | |
| | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDKTHTCPPCPAPEAAGGPSV | |
| | FLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALPAPIEKTISKAKGQPREPQVYTLPPCRDELTK | |
| | NQVSLWCLVKGFYPSDIAVEWESNGQPENNYKTTPPVLDS | |
| | DGSFFLYSKLTVDKSRWQQGNVFSCSVMHEALHNHYTQK | |
| | SLSLSP | |

Anti-P329G (VH3VL1) x anti-CD28 (TGN1412_variant 8) 2 + 1_inverted TCB

| | | |
|---|---|---|
| LC1 | QAVVTQEPSLTVSPGGTVTLTCRSSTGAVTTSNYANWVQE | 89 |
| PG | KPDHLFTGLIGGTNKRAPGTPARFSGSLLGGKAALTLSGAQ | |
| | PEDEAEYYCALWYSNHWVFGGGTKLTVLGQPKAAPSVTL | |
| | FPPSSKKLQANKATLVCLISDFYPGAVTVAWKADSSPVKA | |
| | GVETTTPSKQSNNKYAASSYLSLTPEQWKSHRSYSCQVTH | |
| | EGSTVEKTVAPTECS | |
| LC2 | QVQLVQSGAEVKKPGASVKVSCKASGYTFTSYYIHWVRQ | 108 |
| CD28 | APGQGLEWIGSIYPGNVQTNYNEKFKDRATLTVDTSISTAY | |
| | MELSRLRSDDTAVYFCTRSHYGLDFNFDVWGQGTTVTVSS | |
| | ASVAAPSVFIFPPSDEQLKSGTASVVCLLNNFYPREAKVQW | |
| | KVDNALQSGNSQESVTEQDSKDSTYSLSSTLTLSKADYEK | |
| | HKVYACEVTHQGLSSPVTKSFNRGEC | |
| HC1 | EVQLVESGGGLVQPGGSLRLSCAASGFTFSRYWMNWVRQ | 90 |
| PG | APGKGLEWVGEITPDSSTINYAPSLKGRFTISRDNAKNSLYL | |
| hole | QMNSLRAEDTAVYYCARPYDYGAWFASWGQGTLVTVSS | |
| | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDKTHTCPPCPAPEAAGGPSV | |
| | FLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALPAPIEKTISKAKGQPREPQVCTLPPSRDELTKN | |
| | QVSLSCAVKGFYPSDIAVEWESNGQPENNYKTTPPVLDSD | |
| | GSFFLVSKLTVDKSRWQQGNVFSCSVMHEALHNHYTQKS | |
| | LSLSP | |
| HC2 | EVQLVESGGGLVQPGGSLRLSCAASGFTFSRYWMNWVRQ | 114 |
| PG | APGKGLEWVGEITPDSSTINYAPSLKGRFTISRDNAKNSLYL | |
| CD28 | QMNSLRAEDTAVYYCARPYDYGAWFASWGQGTLVTVSS | |
| knob | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDGGGGSGGGGGDIQMTQSPS | |
| | SLSASVGDRVTITCHASQNIYVYLNWYQQKPGKAPKLLIY | |
| | KASNLHTGVPSRFSGSGSGTDFTLTISSLQPEDFATYYCQQG | |
| | QTYPYTFGGGTKVEIKSSASTKGPSVFPLAPSSKSTSGGTAA | |
| | LGCLVKDYFPEPVTVSWNSGALTSGVHTFPAVLQSSGLYSL | |
| | SSVVTVPSSSLGTQTYICNVNHKPSNTKVDKKVEPKSCDKT | |
| | HTCPPCPAPEAAGGPSVFLFPPKPKDTLMISRTPEVTCVVVD | |
| | VSHEDPEVKFNWYVDGVEVHNAKTKPREEQYNSTYRVVS | |
| | VLTVLHQDWLNGKEYKCKVSNKALPAPIEKTISKAKGQPR | |
| | EPQVYTLPPCRDELTKNQVSLWCLVKGFYPSDIAVEWESN | |
| | GQPENNYKTTPPVLDSDGSFFLYSKLTVDKSRWQQGNVFS | |
| | CSVMHEALHNHYTQKSLSLSP | |

Anti-P329G (VH3xVL1)-anti-CD28 (TGN1412_variant 8) 2 + 1_classical TCB

| | | |
|---|---|---|
| LC1 | QAVVTQEPSLTVSPGGTVTLTCRSSTGAVTTSNYANWVQE | 89 |
| PG | KPDHLFTGLIGGTNKRAPGTPARFSGSLLGGKAALTLSGAQ | |
| | PEDEAEYYCALWYSNHWVFGGGTKLTVLGQPKAAPSVTL | |
| | FPPSSKKLQANKATLVCLISDFYPGAVTVAWKADSSPVKA | |
| | GVETTTPSKQSNNKYAASSYLSLTPEQWKSHRSYSCQVTH | |
| | EGSTVEKTVAPTECS | |
| LC2 | QVQLVQSGAEVKKPGASVKVSCKASGYTFTSYYIHWVRQ | 108 |
| CD28 | APGQGLEWIGSIYPGNVQTNYNEKFKDRATLTVDTSISTAY | |
| | MELSRLRSDDTAVYFCTRSHYGLDFNFDVWGQGTTVTVSS | |
| | ASVAAPSVFIFPPSDEQLKSGTASVVCLLNNFYPREAKVQW | |
| | KVDNALQSGNSQESVTEQDSKDSTYSLSSTLTLSKADYEK | |
| | HKVYACEVTHQGLSSPVTKSFNRGEC | |
| HC1 | EVQLVESGGGLVQPGGSLRLSCAASGFTFSRYWMNWVRQ | 90 |
| PG | APGKGLEWVGEITPDSSTINYAPSLKGRFTISRDNAKNSLYL | |
| hole | QMNSLRAEDTAVYYCARPYDYGAWFASWGQGTLVTVSS | |
| | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDKTHTCPPCPAPEAAGGPSV | |
| | FLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALPAPIEKTISKAKGQPREPQVCTLPPSRDELTKN | |
| | QVSLSCAVKGFYPSDIAVEWESNGQPENNYKTTPPVLDSD | |
| | GSFFLVSKLTVDKSRWQQGNVFSCSVMHEALHNHYTQKS | |
| | LSLSP | |
| HC2 | DIQMTQSPSSLSASVGDRVTITCHASQNIYVYLNWYQQKPG | 115 |
| PG | KAPKLLIYKASNLHTGVPSRFSGSGSGTDFTLTISSLQPEDF | |
| CD28 | ATYYCQQGQTYPYTFGGGTKVEIKSSASTKGPSVFPLAPSS | |
| knob | KSTSGGTAALGCLVKDYFPEPVTVSWNSGALTSGVHTFPA | |
| | VLQSSGLYSLSSVVTVPSSSLGTQTYICNVNHKPSNTKVDK | |
| | KVEPKSCDGGGGSGGGGGEVQLVESGGGLVQPGGSLRLSC | |
| | AASGFTFSRYWMNWVRQAPGKGLEWVGEITPDSSTINYAP | |
| | SLKGRFTISRDNAKNSLYLQMNSLRAEDTAVYYCARPYDY | |
| | GAWFASWGQGTLVTVSSASTKGPSVFPLAPSSKSTSGGTA | |
| | ALGCLVEDYFPEPVTVSWNSGALTSGVHTFPAVLQSSGLYS | |
| | LSSVVTVPSSSLGTQTYICNVNHKPSNTKVDEKVEPKSCDK | |
| | THTCPPCPAPEAAGGPSVFLFPPKPKDTLMISRTPEVTCVVV | |
| | DVSHEDPEVKFNWYVDGVEVHNAKTKPREEQYNSTYRVV | |
| | SVLTVLHQDWLNGKEYKCKVSNKALPAPIEKTISKAKGQP | |
| | REPQVYTLPPCRDELTKNQVSLWCLVKGFYPSDIAVEWES | |
| | NGQPENNYKTTPPVLDSDGSFFLYSKLTVDKSRWQQGNVF | |
| | SCSVMHEALHNHYTQKSLSLSP | |

Exemplary immune activating Fc binding molecules comprising an IL2 variant
Anti-P329G (M-1.7.24) x IL2v huIgG1

| | | |
|---|---|---|
| LC | QAVVTQESALTTSPGETVTLTCRSSTGAVTTSNYANWVQE | 86 |
| | KPDHLFTGLIGGTNKRAPGVPARFSGSLIGDKAALTITGAQ | |
| | TEDEAIYFCALWYSNHWVFGGGTKLTVLGQPKAAPSVTLF | |
| | PPSSKKLQANKATLVCLISDFYPGAVTVAWKADSSPVKAG | |
| | VETTTPSKQSNNKYAASSYLSLTPEQWKSHRSYSCQVTHE | |
| | GSTVEKTVAPTECS | |
| HC1 | DKTHTCPPCPAPEAAGGPSVFLFPPKPKDTLMISRTPEVTCV | 116 |
| IL2v | VVDVSHEDPEVKFNWYVDGVEVHNAKTKPREEQYNSTYR | |
| knob | VVSVLTVLHQDWLNGKEYKCKVSNKALPAPIEKTISKAKG | |
| | QPREPQVYTLPPCRDELTKNQVSLWCLVKGFYPSDIAVEW | |
| | ESNGQPENNYKTTPPVLDSDGSFFLYSKLTVDKSRWQQGN | |
| | VFSCSVMHEALHNHYTQKSLSLSPGGGGGSGGGGSGGGGS | |
| | APASSSTKKTQLQLEHLLLDLQMILNGINNYKNPKLTRMLT | |
| | AKFAMPKKATELKHLQCLEEELKPLEEVLNGAQSKNFHLR | |
| | PRDLISNINVIVLELKGSETTFMCEYADETATIVEFLNRWITF | |
| | AQSIISTLT | |
| HC2 | EVKLLESGGGLVQPGGSLKLSCAASGFDFSRYWMNWVRQ | 88 |
| hole | APGKGLEWIGEITPDSSTINYTPSLKDKFIISRDNAKNTLYLQ | |
| | MIKVRSEDTALYYCVRPYDYGAWFASWGQGTLVTVSAAS | |
| | TKGPSVFPLAPSSKSTSGGTAALGCLVEDYFPEPVTVSWNS | |
| | GALTSGVHTFPAVLQSSGLYSLSSVVTVPSSSLGTQTYICNV | |
| | NHKPSNTKVDEKVEPKSCDKTHTCPPCPAPEAAGGPSVFLF | |
| | PPKPKDTLMISRTPEVTCVVVDVSHEDPEVKFNWYVDGVE | |
| | VHNAKTKPREEQYNSTYRVVSVLTVLHQDWLNGKEYKCK | |
| | VSNKALPAPIEKTISKAKGQPREPQVCTLPPSRDELTKNQVS | |
| | LSCAVKGFYPSDIAVEWESNGQPENNYKTTPPVLDSDGSFF | |
| | LVSKLTVDKSRWQQGNVFSCSVMHEALHNHYTQKSLSLSP | |

Anti-P329G (VH3VL1) x IL2v huIgG1

| | | |
|---|---|---|
| LC | QAVVTQEPSLTVSPGGTVTLTCRSSTGAVTTSNYANWVQE | 15 |
| | KPDHLFTGLIGGTNKRAPGTPARFSGSLLGGKAALTLSGAQ | |
| | PEDEAEYYCALWYSNHWVFGGGTKLTVLGQPKAAPSVTL | |
| | FPPSSEELQANKATLVCLISDFYPGAVTVAWKADSSPVKAG | |
| | VETTTPSKQSNNKYAASSYLSLTPEQWKSHRSYSCQVTHE | |
| | GSTVEKTVAPTECS | |
| HC1 | DKTHTCPPCPAPEAAGGPSVFLFPPKPKDTLMISRTPEVTCV | 116 |
| IL2v | VVDVSHEDPEVKFNWYVDGVEVHNAKTKPREEQYNSTYR | |
| knob | VVSVLTVLHQDWLNGKEYKCKVSNKALPAPIEKTISKAKG | |
| | QPREPQVYTLPPCRDELTKNQVSLWCLVKGFYPSDIAVEW | |
| | ESNGQPENNYKTTPPVLDSDGSFFLYSKLTVDKSRWQQGN | |
| | VFSCSVMHEALHNHYTQKSLSLSPGGGGGSGGGGSGGGGS | |
| | APASSSTKKTQLQLEHLLLDLQMILNGINNYKNPKLTRMLT | |
| | AKFAMPKKATELKHLQCLEEELKPLEEVLNGAQSKNFHLR | |
| | PRDLISNINVIVLELKGSETTFMCEYADETATIVEFLNRWITF | |
| | AQSIISTLT | |
| HC2 hole | EVQLVESGGGLVQPGGSLRLSCAASGFTFSRYWMNWVRQ | 90 |
| | APGKGLEWVGEITPDSSTINYAPSLKGRFTISRDNAKNSLYL | |
| | QMNSLRAEDTAVYYCARPYDYGAWFASWGQGTLVTVSS | |
| | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDKTHTCPPCPAPEAAGGPSV | |
| | FLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALPAPIEKTISKAKGQPREPQVCTLPPSRDELTKN | |
| | QVSLSCAVKGFYPSDIAVEWESNGQPENNYKTTPPVLDSD | |
| | GSFFLVSKLTVDKSRWQQGNVFSCSVMHEALHNHYTQKS | |
| | LSLSP | |

Exemplary immune activating Fc binding molecules comprising 4-1BBL ectodomains
Exemplary sequences of 4-BBL ectodomains

| | | |
|---|---|---|
| Human (hu) | REGPELSPDDPAGLLDLRQGMFAQLVAQNVLLIDGPLSWY | 117 |
| 4-1BBL (71- | SDPGLAGVSLTGGLSYKEDTKELVVAKAGVYYVFFQLELR | |
| 254) | RVVAGEGSGSVSLALHLQPLRSAAGAAALALTVDLPPASS | |
| | EARNSAFGFQGRLLHLSAGQRLGVHLHTEARARHAWQLT | |
| | QGATVLGLFRVTPEIPAGLPSPRSE | |
| hu 4-1BBL | LDLRQGMFAQLVAQNVLLIDGPLSWYSDPGLAGVSLTGGL | 118 |
| (85-254) | SYKEDTKELVVAKAGVYYVFFQLELRRVVAGEGSGSVSLA | |
| | LHLQPLRSAAGAAALALTVDLPPASSEARNSAFGFQGRLLH | |
| | LSAGQRLGVHLHTEARARHAWQLTQGATVLGLFRVTPEIP | |
| | AGLPSPRSE | |
| hu 4-1BBL | DPAGLLDLRQGMFAQLVAQNVLLIDGPLSWYSDPGLAGVS | 119 |
| (80-254) | LTGGLSYKEDTKELVVAKAGVYYVFFQLELRRVVAGEGS | |
| | GSVSLALHLQPLRSAAGAAALALTVDLPPASSEARNSAFGF | |
| | QGRLLHLSAGQRLGVHLHTEARARHAWQLTQGATVLGLF | |
| | RVTPEIPAGLPSPRSE | |
| hu 4-1BBL | PWAVSGARASPGSAASPRLREGPELSPDDPAGLLDLRQGM | 120 |
| (52-254) | FAQLVAQNVLLIDGPLSWYSDPGLAGVSLTGGLSYKEDTK | |
| | ELVVAKAGVYYVFFQLELRRVVAGEGSGSVSLALHLQPLR | |
| | SAAGAAALALTVDLPPASSEARNSAFGFQGRLLHLSAGQR | |
| | LGVHLHTEARARHAWQLTQGATVLGLFRVTPEIPAGLPSP | |
| | RSE | |
| Human (hu) | REGPELSPDDPAGLLDLRQGMFAQLVAQNVLLIDGPLSWY | 121 |
| 4-1BBL (71- | SDPGLAGVSLTGGLSYKEDTKELVVAKAGVYYVFFQLELR | |
| 248) | RVVAGEGSGSVSLALHLQPLRSAAGAAALALTVDLPPASS | |
| | EARNSAFGFQGRLLHLSAGQRLGVHLHTEARARHAWQLT | |
| | QGATVLGLFRVTPEIPAGL | |
| hu 4-1BBL | LDLRQGMFAQLVAQNVLLIDGPLSWYSDPGLAGVSLTGGL | 122 |
| (85-248) | SYKEDTKELVVAKAGVYYVFFQLELRRVVAGEGSGSVSLA | |
| | LHLQPLRSAAGAAALALTVDLPPASSEARNSAFGFQGRLLH | |
| | LSAGQRLGVHLHTEARARHAWQLTQGATVLGLFRVTPEIP | |
| | AGL | |
| hu 4-1BBL | DPAGLLDLRQGMFAQLVAQNVLLIDGPLSWYSDPGLAGVS | 123 |
| (80-248) | LTGGLSYKEDTKELVVAKAGVYYVFFQLELRRVVAGEGS | |
| | GSVSLALHLQPLRSAAGAAALALTVDLPPASSEARNSAFGF | |
| | QGRLLHLSAGQRLGVHLHTEARARHAWQLTQGATVLGLF | |
| | RVTPEIPAGL | |
| hu 4-1BBL | PWAVSGARASPGSAASPRLREGPELSPDDPAGLLDLRQGM | 124 |
| (52-248) | FAQLVAQNVLLIDGPLSWYSDPGLAGVSLTGGLSYKEDTK | |
| | ELVVAKAGVYYVFFQLELRRVVAGEGSGSVSLALHLQPLR | |
| | SAAGAAALALTVDLPPASSEARNSAFGFQGRLLHLSAGQR | |
| | LGVHLHTEARARHAWQLTQGATVLGLFRVTPEIPAGL | |
| dimeric hu 4- | REGPELSPDDPAGLLDLRQGMFAQLVAQNVLLIDGPLSWY | 125 |
| 1BBL (71- | SDPGLAGVSLTGGLSYKEDTKELVVAKAGVYYVFFQLELR | |
| 254) | RVVAGEGSGSVSLALHLQPLRSAAGAAALALTVDLPPASS | |
| connected by | EARNSAFGFQGRLLHLSAGQRLGVHLHTEARARHAWQLT | |
| (G4S)2 linker | QGATVLGLFRVTPEIPAGLPSPRSEGGGGSGGGGSREGPELS | |
| | PDDPAGLLDLRQGMFAQLVAQNVLLIDGPLSWYSDPGLAG | |
| | VSLTGGLSYKEDTKELVVAKAGVYYVFFQLELRRVVAGE | |
| | GSGSVSLALHLQPLRSAAGAAALALTVDLPPASSEARNSAF | |
| | GFQGRLLHLSAGQRLGVHLHTEARARHAWQLTQGATVLG | |
| | LFRVTPEIPAGLPSPRSE | |
| dimeric hu 4- | REGPELSPDDPAGLLDLRQGMFAQLVAQNVLLIDGPLSWY | 126 |
| 1BBL (71- | SDPGLAGVSLTGGLSYKEDTKELVVAKAGVYYVFFQLELR | |
| 248) | RVVAGEGSGSVSLALHLQPLRSAAGAAALALTVDLPPASS | |
| connected by | EARNSAFGFQGRLLHLSAGQRLGVHLHTEARARHAWQLT | |
| (G4S)2 linker | QGATVLGLFRVTPEIPAGLGGGGSGGGGSREGPELSPDDPA | |
| | GLLDLRQGMFAQLVAQNVLLIDGPLSWYSDPGLAGVSLTG | |
| | GLSYKEDTKELVVAKAGVYYVFFQLELRRVVAGEGSGSVS | |
| | LALHLQPLRSAAGAAALALTVDLPPASSEARNSAFGFQGRL | |
| | LHLSAGQRLGVHLHTEARARHAWQLTQGATVLGLFRVTP | |
| | EIPAGL | |
| dimeric hu 4- | DPAGLLDLRQGMFAQLVAQNVLLIDGPLSWYSDPGLAGVS | 127 |
| 1BBL (80- | LTGGLSYKEDTKELVVAKAGVYYVFFQLELRRVVAGEGS | |
| 254) | GSVSLALHLQPLRSAAGAAALALTVDLPPASSEARNSAFGF | |
| connected by | QGRLLHLSAGQRLGVHLHTEARARHAWQLTQGATVLGLF | |
| (G4S)2 linker | RVTPEIPAGLPSPRSEGGGGSGGGGSDPAGLLDLRQGMFAQ | |
| | LVAQNVLLIDGPLSWYSDPGLAGVSLTGGLSYKEDTKELV | |
| | VAKAGVYYVFFQLELRRVVAGEGSGSVSLALHLQPLRSAA | |
| | GAAALALTVDLPPASSEARNSAFGFQGRLLHLSAGQRLGV | |
| | HLHTEARARHAWQLTQGATVLGLFRVTPEIPAGLPSPRSE | |
| dimeric hu 4- | PWAVSGARASPGSAASPRLREGPELSPDDPAGLLDLRQGM | 128 |
| 1BBL (52- | FAQLVAQNVLLIDGPLSWYSDPGLAGVSLTGGLSYKEDTK | |
| 254) | ELVVAKAGVYYVFFQLELRRVVAGEGSGSVSLALHLQPLR | |
| connected by | SAAGAAALALTVDLPPASSEARNSAFGFQGRLLHLSAGQR | |
| (G4S)2 linker | LGVHLHTEARARHAWQLTQGATVLGLFRVTPEIPAGLPSP | |
| | RSEGGGGSGGGGSPWAVSGARASPGSAASPRLREGPELSP | |
| | DDPAGLLDLRQGMFAQLVAQNVLLIDGPLSWYSDPGLAG | |
| | VSLTGGLSYKEDTKELVVAKAGVYYVFFQLELRRVVAGE | |
| | GSGSVSLALHLQPLRSAAGAAALALTVDLPPASSEARNSAF | |
| | GFQGRLLHLSAGQRLGVHLHTEARARHAWQLTQGATVLG | |
| | LFRVTPEIPAGLPSPRSE | |

anti-P329G(M-1.7.24) x 4-1BBL hulgG1. *charged residues

| | | |
|---|---|---|
| LC | QAVVTQESALTTSPGETVTLTCRSSTGAVTTSNYANWVQE | 10 |
| | KPDHLFTGLIGGTNKRAPGVPARFSGSLIGDKAALTITGAQ | |
| | TEDEAIYFCALWYSNHWVFGGGTKLTVLGQPKAAPSVTLF | |
| | PPSSEELQANKATLVCLISDFYPGAVTVAWKADSSPVKAG | |
| | VETTTPSKQSNNKYAASSYLSLTPEQWKSHRSYSCQVTHE | |
| | GSTVEKTVAPTECS | |
| Monomeric | REGPELSPDDPAGLLDLRQGMFAQLVAQNVLLIDGPLSWY | 129 |
| 4-1BBL- | SDPGLAGVSLTGGLSYKEDTKELVVAKAGVYYVFFQLELR | |
| CH1* | RVVAGEGSGSVSLALHLQPLRSAAGAAALALTVDLPPASS | |
| | EARNSAFGFQGRLLHLSAGQRLGVHLHTEARARHAWQLT | |
| | QGATVLGLFRVTPEIPAGLGGGGSGGGGSASTKGPSVFPLA | |
| | PSSKSTSGGTAALGCLVEDYFPEPVTVSWNSGALTSGVHTF | |
| | PAVLQSSGLYSLSSVVTVPSSSLGTQTYICNVNHKPSNTKV | |
| | DEKVEPKSC | |
| Dimeric 4- | REGPELSPDDPAGLLDLRQGMFAQLVAQNVLLIDGPLSWY | 130 |
| 1BBL-CL* | SDPGLAGVSLTGGLSYKEDTKELVVAKAGVYYVFFQLELR | |
| Fc knob | RVVAGEGSGSVSLALHLQPLRSAAGAAALALTVDLPPASS | |
| chain | EARNSAFGFQGRLLHLSAGQRLGVHLHTEARARHAWQLT | |
| | QGATVLGLFRVTPEIPAGLGGGGSGGGGSREGPELSPDDPA | |
| | GLLDLRQGMFAQLVAQNVLLIDGPLSWYSDPGLAGVSLTG | |
| | GLSYKEDTKELVVAKAGVYYVFFQLELRRVVAGEGSGSVS | |
| | LALHLQPLRSAAGAAALALTVDLPPASSEARNSAFGFQGRL | |
| | LHLSAGQRLGVHLHTEARARHAWQLTQGATVLGLFRVTP | |
| | EIPAGLGGGGSGGGGSRTVAAPSVFIFPPSDRKLKSGTASV | |
| | VCLLNNFYPREAKVQWKVDNALQSGNSQESVTEQDSKDS | |
| | TYSLSSTLTLSKADYEKHKVYACEVTHQGLSSPVTKSFNRG | |
| | ECDKTHTCPPCPAPEAAGGPSVFLFPPKPKDTLMISRTPEVT | |
| | CVVVDVSHEDPEVKFNWYVDGVEVHNAKTKPREEQYNST | |
| | YRVVSVLTVLHQDWLNGKEYKCKVSNKALPAPIEKTISKA | |
| | KGQPREPQVYTLPPCRDELTKNQVSLWCLVKGFYPSDIAV | |
| | EWESNGQPENNYKTTPPVLDSDGSFFLYSKLTVDKSRWQQ | |
| | GNVFSCSVMHEALHNHYTQKSLSLSP | |
| anti- | EVKLLESGGGLVQPGGSLKLSCAASGFDFSRYWMNWVRQ | 131 |
| P329G(M- | APGKGLEWIGEITPDSSTINYTPSLKDKFIISRDNAKNTLYLQ | |
| 1.7.24) Fc | MIKVRSEDTALYYCVRPYDYGAWFASWGQGTLVTVSAAS | |
| hole chain | TKGPSVFPLAPSSKSTSGGTAALGCLVKDYFPEPVTVSWNS | |
| | GALTSGVHTFPAVLQSSGLYSLSSVVTVPSSSLGTQTYICNV | |
| | NHKPSNTKVDKKVEPKSCDKTHTCPPCPAPEAAGGPSVFLF | |
| | PPKPKDTLMISRTPEVTCVVVDVSHEDPEVKFNWYVDGVE | |
| | VHNAKTKPREEQYNSTYRVVSVLTVLHQDWLNGKEYKCK | |
| | VSNKALPAPIEKTISKAKGQPREPQVCTLPPSRDELTKNQVS | |
| | LSCAVKGFYPSDIAVEWESNGQPENNYKTTPPVLDSDGSFF | |
| | LVSKLTVDKSRWQQGNVFSCSVMHEALHNHYTQKSLSLSP | |

anti-P329G(VH3VL1) x 4-1BBL hulgG1. *charged residues

| | | |
|---|---|---|
| LC | QAVVTQEPSLTVSPGGTVTLTCRSSTGAVTTSNYANWVQE | 15 |
| | KPDHLFTGLIGGTNKRAPGTPARFSGSLLGGKAALTLSGAQ | |
| | PEDEAEYYCALWYSNHWVFGGGTKLTVLGQPKAAPSVTL | |
| | FPPSSEELQANKATLVCLISDFYPGAVTVAWKADSSPVKAG | |
| | VETTTPSKQSNNKYAASSYLSLTPEQWKSHRSYSCQVTHE | |
| | GSTVEKTVAPTECS | |
| Monomeric | REGPELSPDDPAGLLDLRQGMFAQLVAQNVLLIDGPLSWY | 129 |
| 4-1BBL - | SDPGLAGVSLTGGLSYKEDTKELVVAKAGVYYVFFQLELR | |
| CH1* | RVVAGEGSGSVSLALHLQPLRSAAGAAALALTVDLPPASS | |
| | EARNSAFGFQGRLLHLSAGQRLGVHLHTEARARHAWQLT | |
| | QGATVLGLFRVTPEIPAGLGGGGSGGGGSASTKGPSVFPLA | |
| | PSSKSTSGGTAALGCLVEDYFPEPVTVSWNSGALTSGVHTF | |
| | PAVLQSSGLYSLSSVVTVPSSSLGTQTYICNVNHKPSNTKV | |
| | DEKVEPKSC | |
| Dimeric 4- | REGPELSPDDPAGLLDLRQGMFAQLVAQNVLLIDGPLSWY | 130 |
| 1BBL-CL* | SDPGLAGVSLTGGLSYKEDTKELVVAKAGVYYVFFQLELR | |
| Fc knob | RVVAGEGSGSVSLALHLQPLRSAAGAAALALTVDLPPASS | |
| chain | EARNSAFGFQGRLLHLSAGQRLGVHLHTEARARHAWQLT | |
| | QGATVLGLFRVTPEIPAGLGGGGSGGGGSREGPELSPDDPA | |
| | GLLDLRQGMFAQLVAQNVLLIDGPLSWYSDPGLAGVSLTG | |
| | GLSYKEDTKELVVAKAGVYYVFFQLELRRVVAGEGSGSVS | |
| | LALHLQPLRSAAGAAALALTVDLPPASSEARNSAFGFQGRL | |
| | LHLSAGQRLGVHLHTEARARHAWQLTQGATVLGLFRVTP | |
| | EIPAGLGGGGSGGGGSRTVAAPSVFIFPPSDRKLKSGTASV | |
| | VCLLNNFYPREAKVQWKVDNALQSGNSQESVTEQDSKDS | |
| | TYSLSSTLTLSKADYEKHKVYACEVTHQGLSSPVTKSFNRG | |
| | ECDKTHTCPPCPAPEAAGGPSVFLFPPKPKDTLMISRTPEVT | |
| | CVVVDVSHEDPEVKFNWYVDGVEVHNAKTKPREEQYNST | |
| | YRVVSVLTVLHQDWLNGKEYKCKVSNKALPAPIEKTISKA | |
| | KGQPREPQVYTLPPCRDELTKNQVSLWCLVKGFYPSDIAV | |
| | EWESNGQPENNYKTTPPVLDSDGSFFLYSKLTVDKSRWQQ | |
| | GNVFSCSVMHEALHNHYTQKSLSLSP | |
| anti- | EVQLVESGGGLVQPGGSLRLSCAASGFTFSRYWMNWVRQ | 132 |
| P329G(VH3 | APGKGLEWVGEITPDSSTINYAPSLKGRFTISRDNAKNSLYL | |
| VL1) Fc hole | QMNSLRAEDTAVYYCARPYDYGAWFASWGQGTLVTVSS | |
| chain | ASTKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKKVEPKSCDKTHTCPPCPAPEAAGGPSV | |
| | FLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALPAPIEKTISKAKGQPREPQVCTLPPSRDELTKN | |
| | QVSLSCAVKGFYPSDIAVEWESNGQPENNYKTTPPVLDSD | |
| | GSFFLVSKLTVDKSRWQQGNVFSCSVMHEALHNHYTQKS | |
| | LSLSP | |

Exemplary immune activating Fc binding molecules capable of specific binding
to the costimulatory T cell antigen 41BB
CDR definition according to Kabat
anti-4-1BB(20H4.9) binder

| | | |
|---|---|---|
| HCDR1 | GYYWS | 133 |
| HCDR2 | EINHGGYVTYNPSLES | 134 |
| HCDR3 | DYGPGNYDWYFDL | 135 |
| LCDR1 | RASQSVSSYLA | 136 |
| LCDR2 | DASNRAT | 137 |
| LCDR3 | QQRSNWPPALT | 138 |
| VH | QVQLQQWGAGLLKPSETLSLTCAVYGGSFSGYYWSWIRQS | 139 |
| | PEKGLEWIGEINHGGYVTYNPSLESRVTISVDTSKNQFSLKL | |
| | SSVTAADTAVYYCARDYGPGNYDWYFDLWGRGTLVTVSS | |
| VL | EIVLTQSPATLSLSPGERATLSCRASQSVSSYLAWYQQKPG | 140 |
| | QAPRLLIYDASNRATGIPARFSGSGSGTDFTLTISSLEPEDFA | |
| | VYYCQQRSNWPPALTFGGGTKVEIK | |

2 + 1 anti-4-1BB(20H4.9) x anti-P329G(M-1.7.24) huIgG1

| | | |
|---|---|---|
| LC1 | EVKLLESGGGLVQPGGSLKLSCAASGFDFSRYWMNWVRQ | 141 |
| PG | APGKGLEWIGEITPDSSTINYTPSLKDKFIISRDNAKNTLYLQ | |
| crossed | MIKVRSEDTALYYCVRPYDYGAWFASWGQGTLVTVSAAS | |
| | VAAPSVFIFPPSDEQLKSGTASVVCLLNNFYPREAKVQWKV | |
| | DNALQSGNSQESVTEQDSKDSTYSLSSTLTLSKADYEKHK | |
| | VYACEVTHQGLSSPVTKSFNRGEC | |
| LC2 | EIVLTQSPATLSLSPGERATLSCRASQSVSSYLAWYQQKPG | 142 |
| 41BB | QAPRLLIYDASNRATGIPARFSGSGSGTDFTLTISSLEPEDFA | |
| | VYYCQQRSNWPPALTFGGGTKVEIKRTVAAPSVFIFPPSDR | |
| | KLKSGTASVVCLLNNFYPREAKVQWKVDNALQSGNSQES | |
| | VTEQDSKDSTYSLSSTLTLSKADYEKHKVYACEVTHQGLS | |
| | SPVTKSFNRGEC | |
| HC1 | QAVVTQESALTTSPGETVTLTCRSSTGAVTTSNYANWVQE | 143 |
| PG | KPDHLFTGLIGGTNKRAPGVPARFSGSLIGDKAALTITGAQ | |
| 41BB | TEDEAIYFCALWYSNHWVFGGGTKLTVLASTKGPSVFPLA | |
| knob | PSSKSTSGGTAALGCLVKDYFPEPVTVSWNSGALTSGVHTF | |
| | PAVLQSSGLYSLSSVVTVPSSSLGTQTYICNVNHKPSNTKV | |
| VLCH1 (M- | DKKVEPKSCDGGGGSGGGGSQVQLQQWGAGLLKPSETLS | |
| 1.7.24) | LTCAVYGGSFSGYYWSWIRQSPEKGLEWIGEINHGGYVTY | |
| VHCH1(EE) | NPSLESRVTISVDTSKNQFSLKLSSVTAADTAVYYCARDYG | |
| (20H4.9)- | PGNYDWYFDLWGRGTLVTVSSASTKGPSVFPLAPSSKSTS | |
| Heavy chain | GGTAALGCLVEDYFPEPVTVSWNSGALTSGVHTFPAVLQS | |
| HC2 (Fc | SGLYSLSSVVTVPSSSLGTQTYICNVNHKPSNTKVDEKVEP | |
| knob) | KSCDKTHTCPPCPAPEAAGGPSVFLFPPKPKDTLMISRTPEV | |
| | TCVVVDVSHEDPEVKFNWYVDGVEVHNAKTKPREEQYNS | |
| | TYRVVSVLTVLHQDWLNGKEYKCKVSNKALPAPIEKTISK | |
| | AKGQPREPQVYTLPPCRDELTKNQVSLWCLVKGFYPSDIA | |
| | VEWESNGQPENNYKTTPPVLDSDGSFFLYSKLTVDKSRWQ | |
| | QGNVFSCSVMHEALHNHYTQKSLSLSP | |
| HC2 | QVQLQQWGAGLLKPSETLSLTCAVYGGSFSGYYWSWIRQS | 144 |
| 41BB | PEKGLEWIGEINHGGYVTYNPSLESRVTISVDTSKNQFSLKL | |
| hole | SSVTAADTAVYYCARDYGPGNYDWYFDLWGRGTLVTVSS | |
| | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDKTHTCPPCPAPEAAGGPSV | |
| | FLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALPAPIEKTISKAKGQPREPQVCTLPPSRDELTKN | |
| | QVSLSCAVKGFYPSDIAVEWESNGQPENNYKTTPPVLDSD | |
| | GSFFLVSKLTVDKSRWQQGNVFSCSVMHEALHNHYTQKS | |
| | LSLSP | |

2 + 1 anti-4-1BB(20H4.9) x anti-P329G(VH3VL1) huIgG1

| | | |
|---|---|---|
| LC1 | EVQLVESGGGLVQPGGSLRLSCAASGFTFSRYWMNWVRQ | 110 |
| PG | APGKGLEWVGEITPDSSTINYAPSLKGRFTISRDNAKNSLYL | |
| crossed | QMNSLRAEDTAVYYCARPYDYGAWFASWGQGTLVTVSS | |
| | ASVAAPSVFIFPPSDEQLKSGTASVVCLLNNFYPREAKVQW | |
| | KVDNALQSGNSQESVTEQDSKDSTYSLSSTLTLSKADYEK | |
| | HKVYACEVTHQGLSSPVTKSFNRGEC | |
| LC2 | EIVLTQSPATLSLSPGERATLSCRASQSVSSYLAWYQQKPG | 142 |
| 41BB | QAPRLLIYDASNRATGIPARFSGSGSGTDFTLTISSLEPEDFA | |
| | VYYCQQRSNWPPALTFGGGTKVEIKRTVAAPSVFIFPPSDR | |
| | KLKSGTASVVCLLNNFYPREAKVQWKVDNALQSGNSQES | |
| | VTEQDSKDSTYSLSSTLTLSKADYEKHKVYACEVTHQGLS | |
| | SPVTKSFNRGEC | |
| HC1 | QAVVTQEPSLTVSPGGTVTLTCRSSTGAVTTSNYANWVQE | 145 |
| PG | KPDHLFTGLIGGTNKRAPGTPARFSGSLLGGKAALTLSGAQ | |
| knob | PEDEAEYYCALWYSNHWVFGGGTKLTVLASTKGPSVFPLA | |
| | PSSKSTSGGTAALGCLVKDYFPEPVTVSWNSGALTSGVHTF | |
| | PAVLQSSGLYSLSSVVTVPSSSLGTQTYICNVNHKPSNTKV | |
| | DKKVEPKSCDGGGGSGGGGSQVQLQQWGAGLLKPSETLS | |
| | LTCAVYGGSFSGYYWSWIRQSPEKGLEWIGEINHGGYVTY | |
| | NPSLESRVTISVDTSKNQFSLKLSSVTAADTAVYYCARDYG | |
| | PGNYDWYFDLWGRGTLVTVSSASTKGPSVFPLAPSSKSTS | |
| | GGTAALGCLVEDYFPEPVTVSWNSGALTSGVHTFPAVLQS | |
| | SGLYSLSSVVTVPSSSLGTQTYICNVNHKPSNTKVDEKVEP | |
| | KSCDKTHTCPPCPAPEAAGGPSVFLFPPKPKDTLMISRTPEV | |
| | TCVVVDVSHEDPEVKFNWYVDGVEVHNAKTKPREEQYNS | |
| | TYRVVSVLTVLHQDWLNGKEYKCKVSNKALPAPIEKTISK | |
| | AKGQPREPQVYTLPPCRDELTKNQVSLWCLVKGFYPSDIA | |
| | VEWESNGQPENNYKTTPPVLDSDGSFFLYSKLTVDKSRWQ | |
| | QGNVFSCSVMHEALHNHYTQKSLSLSP | |
| HC2 | QVQLQQWGAGLLKPSETLSLTCAVYGGSFSGYYWSWIRQS | 144 |
| 41BB | PEKGLEWIGEINHGGYVTYNPSLESRVTISVDTSKNQFSLKL | |
| hole | SSVTAADTAVYYCARDYGPGNYDWYFDLWGRGTLVTVSS | |
| | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDKTHTCPPCPAPEAAGGPSV | |
| | FLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALPAPIEKTISKAKGQPREPQVCTLPPSRDELTKN | |
| | QVSLSCAVKGFYPSDIAVEWESNGQPENNYKTTPPVLDSD | |
| | GSFFLVSKLTVDKSRWQQGNVFSCSVMHEALHNHYTQKS | |
| | LSLSP | |

Exemplary targeting antibodies
Anti-PD-L1 with P329G mutation

| | | |
|---|---|---|
| HC | EVQLVESGGGLVQPGGSLRLSCAASGFTFSDSWIHWVRQA | 146 |
| | PGKGLEWVAWISPYGGSTYYADSVKGRFTISADTSKNTAY | |
| | LQMNSLRAEDTAVYYCARRHWPGGFDYWGQGTLVTVSS | |
| | ASTKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKKVEPKSCDKTHTCPPCP | |
| | APEAAGGPSVFLFPPKPKDTLMISRTPEVTCVVVDVSHEDP | |
| | EVKFNWYVDGVEVHNAKTKPREEQYASTYRVVSVLTVLH | |
| | QDWLNGKEYKCKVSNKALGAPIEKTISKAKGQPREPQVYT | |
| | LPPSREEMTKNQVSLTCLVKGFYPSDIAVEWESNGQPENN | |
| | YKTTPPVLDSDGSFFLYSKLTVDKSRWQQGNVFSCSVMHE | |
| | ALHNHYTQKSLSLSP | |
| LC | DIQMTQSPSSLSASVGDRVTITCRASQDVSTAVAWYQQKP | 147 |
| | GKAPKLLIYSASFLYSGVPSRFSGSGSGTDFTLTISSLQPEDF | |
| | ATYYCQQYLYHPATFGQGTKVEIKRTVAAPSVFIFPPSDEQ | |
| | LKSGTASVVCLLNNFYPREAKVQWKVDNALQSGNSQESV | |
| | TEQDSKDSTYSLSSTLTLSKADYEKHKVYACEVTHQGLSSP | |
| | VTKSFN | |

Anti-CD20 (GA101) huIgG1 P329G

| | | |
|---|---|---|
| HC | QVQLVQSGAEVKKPGSSVKVSCKASGYAFSYSWINWVRQ | 148 |
| | APGQGLEWMGRIFPGDGDTDYNGKFKGRVTITADKSTSTA | |
| | YMELSSLRSEDTAVYYCARNVFDGYWLVYWGQGTLVTVS | |
| | SASTKGPSVFPLAPSSKSTSGGTAALGCLVKDYFPEPVTVS | |
| | WNSGALTSGVHTFPAVLQSSGLYSLSSVVTVPSSSLGTQTY | |
| | ICNVNHKPSNTKVDKKVEPKSCDKTHTCPPCPAPEAAGGPS | |
| | VFLFPPKPKDTLMISRTPEVTCVVVDVSHEDPEVKFNWYV | |
| | DGVEVHNAKTKPREEQYNSTYRVVSVLTVLHQDWLNGKE | |
| | YKCKVSNKALGAPIEKTISKAKGQPREPQVYTLPPSRDELT | |
| | KNQVSLTCLVKGFYPSDIAVEWESNGQPENNYKTTPPVLD | |
| | SDGSFFLYSKLTVDKSRWQQGNVFSCSVMHEALHNHYTQ | |
| | KSLSLSP | |
| LC | DIVMTQTPLSLPVTPGEPASISCRSSKSLLHSNGITYLYWYL | 149 |
| | QKPGQSPQLLIYQMSNLVSGVPDRFSGSGSGTDFTLKISRVE | |
| | AEDVGVYYCAQNLELPYTFGGGTKVEIKRTVAAPSVFIFPP | |
| | SDEQLKSGTASVVCLLNNFYPREAKVQWKVDNALQSGNS | |
| | QESVTEQDSKDSTYSLSSTLTLSKADYEKHKVYACEVTHQ | |
| | GLSSPVTKSFNRGEC | |

Anti-FolR1 (16D5) huIgG1 P329G

| | | |
|---|---|---|
| HC | EVQLVESGGGLVKPGGSLRLSCAASGFTFSNAWMSWVRQ | 150 |
| | APGKGLEWVGRIKSKTDGGTTDYAAPVKGRFTISRDDSKN | |
| | TLYLQMNSLKTEDTAVYYCTTPWEWSWYDYWGQGTLVT | |
| | VSSASTKGPSVFPLAPSSKSTSGGTAALGCLVKDYFPEPVT | |
| | VSWNSGALTSGVHTFPAVLQSSGLYSLSSVVTVPSSSLGTQ | |
| | TYICNVNHKPSNTKVDKKVEPKSCDKTHTCPPCPAPEAAG | |
| | GPSVFLFPPKPKDTLMISRTPEVTCVVVDVSHEDPEVKFNW | |
| | YVDGVEVHNAKTKPREEQYNSTYRVVSVLTVLHQDWLNG | |
| | KEYKCKVSNKALGAPIEKTISKAKGQPREPQVYTLPPSRDE | |
| | LTKNQVSLTCLVKGFYPSDIAVEWESNGQPENNYKTTPPVL | |
| | DSDGSFFLYSKLTVDKSRWQQGNVFSCSVMHEALHNHYT | |
| | QKSLSLSP | |
| LC | QAVVTQEPSLTVSPGGTVTLTCGSSTGAVTTSNYANWVQE | 151 |
| | KPGQAFRGLIGGTNKRAPGTPARFSGSLLGGKAALTLSGAQ | |
| | PEDEAEYYCALWYSNLWVFGGGTKLTVLGQPKAAPSVTL | |
| | FPPSSEELQANKATLVCLISDFYPGAVTVAWKADSSPVKAG | |
| | VETTTPSKQSNNKYAASSYLSLTPEQWKSHRSYSCQVTHE | |
| | GSTVEKTVAPTECS | |

Anti-CD25 (TSK013044) huIgG1 P329G

| | | |
|---|---|---|
| HC | QVQLVQSGAEVKKPGSSVKVSCKASGGTFSSLAISWVRQA | 152 |
| | PGQGLEWMGGIIPIFGTANYAQKFQGRVTITADESTSTAYM | |
| | ELSSLRSEDTAVYYCARGGSVSGTLVDFDIWGQGTMVTVS | |
| | SASTKGPSVFPLAPSSKSTSGGTAALGCLVKDYFPEPVTVS | |
| | WNSGALTSGVHTFPAVLQSSGLYSLSSVVTVPSSSLGTQTY | |
| | ICNVNHKPSNTKVDKKVEPKSCDKTHTCPPCPAPEAAGGPS | |
| | VFLFPPKPKDTLMISRTPEVTCVVVDVSHEDPEVKFNWYV | |
| | DGVEVHNAKTKPREEQYNSTYRVVSVLTVLHQDWLNGKE | |
| | YKCKVSNKALGAPIEKTISKAKGQPREPQVYTLPPSRDELT | |
| | KNQVSLTCLVKGFYPSDIAVEWESNGQPENNYKTTPPVLD | |
| | SDGSFFLYSKLTVDKSRWQQGNVFSCSVMHEALHNHYTQ | |
| | KSLSLSP | |
| LC | DIQMTQSPSTLSASVGDRVTITCRASQSISSWLAWYQQKPG | 153 |
| | KAPKLLIYKASSLESGVPSRFSGSGSGTEFTLTISSLQPDDFA | |
| | TYYCQQYNIYPITFGGGTKVEIKRTVAAPSVFIFPPSDEQLK | |
| | SGTASVVCLLNNFYPREAKVQWKVDNALQSGNSQESVTE | |
| | QDSKDSTYSLSSTLTLSKADYEKHKVYACEVTHQGLSSPVT | |
| | KSFNRGEC | |

Anti-FAP (4B9) huIgG1 P329G

| | | |
|---|---|---|
| HC | EVQLLESGGGLVQPGGSLRLSCAASGFTFSSYAMSWVRQA | 154 |
| | PGKGLEWVSAIIGSGASTYYADSVKGRFTISRDNSKNTLYL | |
| | QMNSLRAEDTAVYYCAKGWFGGFNYWGQGTLVTVSSAS | |
| | TKGPSVFPLAPSSKSTSGGTAALGCLVKDYFPEPVTVSWNS | |
| | GALTSGVHTFPAVLQSSGLYSLSSVVTVPSSSLGTQTYICNV | |
| | NHKPSNTKVDKKVEPKSCDKTHTCPPCPAPEAAGGPSVFLF | |
| | PPKPKDTLMISRTPEVTCVVVDVSHEDPEVKFNWYVDGVE | |
| | VHNAKTKPREEQYNSTYRVVSVLTVLHQDWLNGKEYKCK | |
| | VSNKALGAPIEKTISKAKGQPREPQVYTLPPSRDELTKNQV | |
| | SLTCLVKGFYPSDIAVEWESNGQPENNYKTTPPVLDSDGSF | |
| | FLYSKLTVDKSRWQQGNVFSCSVMHEALHNHYTQKSLSLS | |
| | P | |
| LC | EIVLTQSPGTLSLSPGERATLSCRASQSVTSSYLAWYQQKP | 155 |
| | GQAPRLLINVGSRRATGIPDRESGSGSGTDFTLTISRLEPEDF | |
| | AVYYCQQGIMLPPTFGQGTKVEIKRTVAAPSVFIFPPSDEQL | |
| | KSGTASVVCLLNNFYPREAKVQWKVDNALQSGNSQESVT | |
| | EQDSKDSTYSLSSTLTLSKADYEKHKVYACEVTHQGLSSPV | |
| | TKSFNRGEC | |

Anti-EpCAM (3-171) huIgG1 P329G

| | | |
|---|---|---|
| HC | QVQLVQSGAEVKKPGSSVKVSCKASGGTFSSYAISWVRQA | 156 |
| | PGQGLEWMGGIIPIFGTANYAQKFQGRVTITADESTSTAYM | |
| | ELSSLRSEDTAVYYCARGLLWNYWGQGTLVTVSSASTKGP | |
| | SVFPLAPSSKSTSGGTAALGCLVEDYFPEPVTVSWNSGALT | |
| | SGVHTFPAVLQSSGLYSLSSVVTVPSSSLGTQTYICNVNHKP | |
| | SNTKVDEKVEPKSCDKTHTCPPCPAPEAAGGPSVFLFPPKP | |
| | KDTLMISRTPEVTCVVVDVSHEDPEVKFNWYVDGVEVHN | |
| | AKTKPREEQYNSTYRVVSVLTVLHQDWLNGKEYKCKVSN | |
| | KALGAPIEKTISKAKGQPREPQVCTLPPSRDELTKNQVSLSC | |
| | AVKGFYPSDIAVEWESNGQPENNYKTTPPVLDSDGSFFLVS | |
| | KLTVDKSRWQQGNVFSCSVMHEALHNHYTQKSLSLSP | |
| LC | EIVMTQSPATLSVSPGERATLSCRASQSVSSNLAWYQQKPG | 157 |
| | QAPRLIIYGASTTASGIPARFSASGSGTDFTLTISSLQSEDFA | |
| | VYYCQQYNNWPPAYTFGQGTKLEIKRTVAAPSVFIFPPSDR | |
| | KLKSGTASVVCLLNNFYPREAKVQWKVDNALQSGNSQES | |
| | VTEQDSKDSTYSLSSTLTLSKADYEKHKVYACEVTHQGLS | |
| | SPVTKSFNRGEC | |

Anti-STEAP1 huIgG1 P329G

| | | |
|---|---|---|
| HC | EVQLVESGGGLVQPGGSLRLSCAVSGYSITSDYAWNWVRQ | 158 |
| | APGKGLEWVGYISNSGSTSYNPSLKSRFTISRDTSKNTLYLQ | |
| | MNSLRAEDTAVYYCARERNYDYEDYYYAMDYWGQGTLV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQ | |
| | TYICNVNHKPSNTKVDKKVEPKSCDKTHTCPPCPAPEAAG | |
| | GPSVFLFPPKPKDTLMISRTPEVTCVVVDVSHEDPEVKENW | |
| | YVDGVEVHNAKTKPREEQYNSTYRVVSVLTVLHQDWLNG | |
| | KEYKCKVSNKALGAPIEKTISKAKGQPREPQVYTLPPSRDE | |
| | LTKNQVSLTCLVKGFYPSDIAVEWESNGQPENNYKTTPPVL | |
| | DSDGSFFLYSKLTVDKSRWQQGNVFSCSVMHEALHNHYT | |
| | QKSLSLSP | |
| LC | DIQMTQSPSSLSASVGDRVTITCKSSQSLLYRSNQKNYLAW | 159 |
| | YQQKPGKAPKLLIYWASTRESGVPSRFSGSGSGTDFTLTISS | |
| | LQPEDFATYYCQQYYNYPRTFGQGTKVEIKRTVAAPSVFIF | |
| | PPSDEQLKSGTASVVCLLNNFYPREAKVQWKVDNALQSG | |
| | NSQESVTEQDSKDSTYSLSSTLTLSKADYEKHKVYACEVT | |
| | HQGLSSPVTKSFNRGEC | |

Anti-PD1 huIgG1 P329G

| | | |
|---|---|---|
| HC | EVQLLESGGGLVQPGGSLRLSCAASGFSFSSYTMSWVRQA | 160 |
| | PGKGLEWVATISGGGRDIYYPDSVKGRFTISRDNSKNTLYL | |
| | QMNSLRAEDTAVYYCVLLTGRVYFALDSWGQGTLVTVSS | |
| | ASTKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKKVEPKSCDKTHTCPPCPAPEAAGGPSV | |
| | FLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALGAPIEKTISKAKGQPREPQVYTLPPSRDELTK | |
| | NQVSLTCLVKGFYPSDIAVEWESNGQPENNYKTTPPVLDS | |
| | DGSFFLYSKLTVDKSRWQQGNVFSCSVMHEALHNHYTQK | |
| | SLSLSP | |
| LC | DIVMTQSPDSLAVSLGERATINCKASESVDTSDNSFIHWYQ | 161 |
| | QKPGQSPKLLIYRSSTLESGVPDRFSGSGSGTDFTLTISSLQA | |
| | EDVAVYYCQQNYDVPWTFGQGTKVEIKRTVAAPSVFIFPP | |
| | SDEQLKSGTASVVCLLNNFYPREAKVQWKVDNALQSGNS | |
| | QESVTEQDSKDSTYSLSSTLTLSKADYEKHKVYACEVTHQ | |
| | GLSSPVTKSFNRGEC | |

anti-Her2 (Pertuzumab) huIgG1 P329G

| | | |
|---|---|---|
| HC | EVQLVESGGGLVQPGGSLRLSCAASGFTFTDYTMDWVRQ | 162 |
| | APGKGLEWVADVNPNSGGSIYNQRFKGRFTLSVDRSKNTL | |
| | YLQMNSLRAEDTAVYYCARNLGPSFYFDYWGQGTLVTVS | |
| | SASTKGPSVFPLAPSSKSTSGGTAALGCLVKDYFPEPVTVS | |
| | WNSGALTSGVHTFPAVLQSSGLYSLSSVVTVPSSSLGTQTY | |
| | ICNVNHKPSNTKVDKKVEPKSCDKTHTCPPCPAPEAAGGPS | |
| | VFLFPPKPKDTLMISRTPEVTCVVVDVSHEDPEVKFNWYV | |
| | DGVEVHNAKTKPREEQYNSTYRVVSVLTVLHQDWLNGKE | |
| | YKCKVSNKALGAPIEKTISKAKGQPREPQVYTLPPSRDELT | |
| | KNQVSLTCLVKGFYPSDIAVEWESNGQPENNYKTTPPVLD | |
| | SDGSFFLYSKLTVDKSRWQQGNVFSCSVMHEALHNHYTQ | |
| | KSLSLSPGK | |
| LC | DIQMTQSPSSLSASVGDRVTITCKASQDVSIGVAWYQQKPG | 163 |
| | KAPKLLIYSASYRYTGVPSRFSGSGSGTDFTLTISSLQPEDFA | |
| | TYYCQQYYIYPYTFGQGTKVEIKRTVAAPSVFIFPPSDEQLK | |
| | SGTASVVCLLNNFYPREAKVQWKVDNALQSGNSQESVTE | |
| | QDSKDSTYSLSSTLTLSKADYEKHKVYACEVTHQGLSSPVT | |
| | KSFNRGEC | |

anti-CEA (T84.66 LCHA) huIgG1 P329G

| | | |
|---|---|---|
| HC | QVQLVQSGAEVKKPGSSVKVSCKASGFNIKDTYMHWVRQ | 164 |
| | APGQGLEWMGRIDPANGNSKYVPKFQGRVTITADTSTSTA | |
| | YMELSSLRSEDTAVYYCAPFGYYVSDYAMAYWGQGTLVT | |
| | VSSASTKGPSVFPLAPSSKSTSGGTAALGCLVKDYFPEPVT | |
| | VSWNSGALTSGVHTFPAVLQSSGLYSLSSVVTVPSSSLGTQ | |
| | TYICNVNHKPSNTKVDKKVEPKSCDKTHTCPPCPAPEAAG | |
| | GPSVFLFPPKPKDTLMISRTPEVTCVVVDVSHEDPEVKFNW | |
| | YVDGVEVHNAKTKPREEQYNSTYRVVSVLTVLHQDWLNG | |
| | KEYKCKVSNKALGAPIEKTISKAKGQPREPQVYTLPPSRDE | |
| | LTKNQVSLTCLVKGFYPSDIAVEWESNGQPENNYKTTPPVL | |
| | DSDGSFFLYSKLTVDKSRWQQGNVFSCSVMHEALHNHYT | |
| | QKSLSLSPGK | |
| LC | EIVLTQSPATLSLSPGERATLSCRAGESVDIFGVGFLHWYQ | 165 |
| | QKPGQAPRLLIYRASNRATGIPARFSGSGSGTDFTLTISSLEP | |
| | EDFAVYYCQQTNEDPYTFGQGTKLEIKRTVAAPSVFIFPPS | |
| | DEQLKSGTASVVCLLNNFYPREAKVQWKVDNALQSGNSQ | |
| | ESVTEQDSKDSTYSLSSTLTLSKADYEKHKVYACEVTHQG | |
| | LSSPVTKSFNRGEC | |

Exemplary IL2 sequences
IL2v

| | | |
|---|---|---|
| human IL2 | APTSSSTKKTQLQLEHLLLDLQMILNGINNYKNPKLTRMLT | 166 |
| | FKFYMPKKATELKHLQCLE | |
| | EELKPLEEVLNLAQSKNFHLRPRDLISNINVIVLELKGSETTF | |
| | MCEYADETATIVEFLNR | |
| | WITFCQSIISTLT | |
| IL2v | APASSSTKKTQLQLEHLLLDLQMILNGINNYKNPKLTRMLT | 167 |
| | AKFAMPKKATELKHLQCLEEELKPLEEVLNGAQSKNFHLR | |
| | PRDLISNINVIVLELKGSETTFMCEYADETATIVEFLNRWITF | |
| | AQSIISTLT | |

anti-AAA binder

| | | |
|---|---|---|
| HCDR1 | GYYWS | 168 |
| HCDR2 | EINHGGYVTYNPSLES | 169 |
| HCDR3 | DYGPGNYDWYFDL | 170 |
| LCDR1 | RASQSVSSYLA | 171 |
| LCDR2 | DASNRAT | 172 |
| LCDR3 | QQRSNWPPALT | 173 |
| VH | MNFGLSLVFLALILKGVQCEVQLVESGGDLVKPGGSLKLS | 174 |
| | CAASGFTFSSYGMSWVRQTPDKRLEWVATISSGGSYIYYP | |
| | DSVKGRFTISRDNAKNTLYLQMSSLKSEDTAMYYCARLG | |
| | MITTGYAMDYWGQGTSVTVSS | |
| VL | DVLMTQTPLSLPVSLGDQASISCRSSQTIVHSTGHTYLEWFL | 175 |
| | QKPGQSPKLLIYKVSNRFSGVPDRFSGSGSGTDFTLKISRVE | |
| | AEDLGVYYCFQGSHVPYTFGGGTKLEIK | |

Improved CD3 binder
CD3 clone 22

| | | |
|---|---|---|
| HCDR1 | SYAMN | 35 |
| HCDR2 | RIRSKYNNYATYYADSVKG | 37 |
| HCDR3 | HTTFPSSYVSYYGY | 176 |
| VH | EVQLLESGGGLVQPGGSLRLSCAASGFQFSSYAMNWVRQA | 177 |
| | PGKGLEWVSRIRSKYNNYATYYADSVKGRFTISRDDSKNT | |
| | LYLQMNSLRAEDTAVYYCVRHTTFPSSYVSYYGYWGQGT | |
| | LVTVSS | |
| LCDR1 | GSSTGAVTTSNYAN | 53 |
| LCDR2 | GTNKRAP | 54 |
| LCDR3 | ALWYSNLWV | 55 |
| VL | QAVVTQEPSLTVSPGGTVTLTCGSSTGAVTTSNYANWVQE | 56 |
| | KPGQAFRGLIGGTNKRAPGTPARFSGSLLGGKAALTLSGAQ | |
| | PEDEAEYYCALWYSNLWVFGGGTKLTVL | |

Exemplary immune activating Fc binding molecules capable of specific binding
to the activating T cell antigen CD3
Anti-P329G (VH3VL1) x anti-CD3 (P035.093) 2 + 1 TCB, P329R LALA Fc

| | | |
|---|---|---|
| LC1 | QAVVTQEPSLTVSPGGTVTLTCRSSTGAVTTSNYANWVQE | 89 |
| PG | KPDHLFTGLIGGTNKRAPGTPARFSGSLLGGKAALTLSGAQ | |
| | PEDEAEYYCALWYSNHWVFGGGTKLTVLGQPKAAPSVTL | |
| | FPPSSKKLQANKATLVCLISDFYPGAVTVAWKADSSPVKA | |
| | GVETTTPSKQSNNKYAASSYLSLTPEQWKSHRSYSCQVTH | |
| | EGSTVEKTVAPTECS | |
| LC2 | EVQLLESGGGLVQPGGSLRLSCAASGFTFSSYAMNWVRQA | 70 |
| CD3 | PGKGLEWVSRIRSKYNNYATYYADSVKGRFTISRDDSKNT | |
| (crossed) | LYLQMNSLRAEDTAVYYCVRASNFPASYVSYFAYWGQGT | |
| | LVTVSSASVAAPSVFIFPPSDEQLKSGTASVVCLLNNFYPRE | |
| | AKVQWKVDNALQSGNSQESVTEQDSKDSTYSLSSTLTLSK | |
| | ADYEKHKVYACEVTHQGLSSPVTKSFNRGEC | |
| HC1 | EVQLVESGGGLVQPGGSLRLSCAASGFTFSRYWMNWVRQ | 178 |
| PG | APGKGLEWVGEITPDSSTINYAPSLKGRFTISRDNAKNSLYL | |
| hole | QMNSLRAEDTAVYYCARPYDYGAWFASWGQGTLVTVSS | |
| | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDKTHTCPPCPAPEAAGGPSV | |
| | FLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALRAPIEKTISKAKGQPREPQVCTLPPSRDELTK | |
| | NQVSLSCAVKGFYPSDIAVEWESNGQPENNYKTTPPVLDS | |
| | DGSFFLVSKLTVDKSRWQQGNVFSCSVMHEALHNHYTQK | |
| | SLSLSP | |
| HC2 | EVQLVESGGGLVQPGGSLRLSCAASGFTFSRYWMNWVRQ | 179 |
| PG | APGKGLEWVGEITPDSSTINYAPSLKGRFTISRDNAKNSLYL | |
| CD3 | QMNSLRAEDTAVYYCARPYDYGAWFASWGQGTLVTVSS | |
| knob | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDGGGGSGGGGGQAVVTQEP | |
| | SLTVSPGGTVTLTCGSSTGAVTTSNYANWVQEKPGQAFRG | |
| | LIGGTNKRAPGTPARFSGSLLGGKAALTLSGAQPEDEAEYY | |
| | CALWYSNLWVFGGGTKLTVLSSASTKGPSVFPLAPSSKSTS | |
| | GGTAALGCLVKDYFPEPVTVSWNSGALTSGVHTFPAVLQS | |
| | SGLYSLSSVVTVPSSSLGTQTYICNVNHKPSNTKVDKKVEP | |
| | KSCDKTHTCPPCPAPEAAGGPSVFLFPPKPKDTLMISRTPEV | |
| | TCVVVDVSHEDPEVKFNWYVDGVEVHNAKTKPREEQYNS | |
| | TYRVVSVLTVLHQDWLNGKEYKCKVSNKALRAPIEKTISK | |
| | AKGQPREPQVYTLPPCRDELTKNQVSLWCLVKGFYPSDIA | |
| | VEWESNGQPENNYKTTPPVLDSDGSFFLYSKLTVDKSRWQ | |
| | QGNVFSCSVMHEALHNHYTQKSLSLSP | |

Anti-P329G (VH3VL1) x anti-CD3 (CH2527) 2 + 1 TCB, P329R LALA Fc

| | | |
|---|---|---|
| LC1 | QAVVTQEPSLTVSPGGTVTLTCRSSTGAVTTSNYANWVQE | 89 |
| PG | KPDHLFTGLIGGTNKRAPGTPARFSGSLLGGKAALTLSGAQ | |
| | PEDEAEYYCALWYSNHWVFGGGTKLTVLGQPKAAPSVTL | |
| | FPPSSKKLQANKATLVCLISDFYPGAVTVAWKADSSPVKA | |
| | GVETTTPSKQSNNKYAASSYLSLTPEQWKSHRSYSCQVTH | |
| | EGSTVEKTVAPTECS | |
| LC2 | EVQLLESGGGLVQPGGSLRLSCAASGFTESTYAMNWVRQA | 68 |
| CD3 | PGKGLEWVSRIRSKYNNYATYYADSVKGRFTISRDDSKNT | |
| (crossed) | LYLQMNSLRAEDTAVYYCVRHGNFGNSYVSWFAYWGQG | |
| | TLVTVSSASVAAPSVFIFPPSDEQLKSGTASVVCLLNNFYPR | |
| | EAKVQWKVDNALQSGNSQESVTEQDSKDSTYSLSSTLTLS | |
| | KADYEKHKVYACEVTHQGLSSPVTKSFNRGEC | |
| HC1 | EVQLVESGGGLVQPGGSLRLSCAASGFTFSRYWMNWVRQ | 178 |
| PG | APGKGLEWVGEITPDSSTINYAPSLKGRFTISRDNAKNSLYL | |
| hole | QMNSLRAEDTAVYYCARPYDYGAWFASWGQGTLVTVSS | |
| | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDKTHTCPPCPAPEAAGGPSV | |
| | FLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALRAPIEKTISKAKGQPREPQVCTLPPSRDELTK | |
| | NQVSLSCAVKGFYPSDIAVEWESNGQPENNYKTTPPVLDS | |
| | DGSFFLVSKLTVDKSRWQQGNVFSCSVMHEALHNHYTQK | |
| | SLSLSP | |
| HC2 | EVQLVESGGGLVQPGGSLRLSCAASGFTFSRYWMNWVRQ | 179 |
| PG | APGKGLEWVGEITPDSSTINYAPSLKGRFTISRDNAKNSLYL | |
| CD3 | QMNSLRAEDTAVYYCARPYDYGAWFASWGQGTLVTVSS | |
| knob | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDGGGGSGGGGGQAVVTQEP | |
| | SLTVSPGGTVTLTCGSSTGAVTTSNYANWVQEKPGQAFRG | |
| | LIGGTNKRAPGTPARFSGSLLGGKAALTLSGAQPEDEAEYY | |
| | CALWYSNLWVFGGGTKLTVLSSASTKGPSVFPLAPSSKSTS | |
| | GGTAALGCLVKDYFPEPVTVSWNSGALTSGVHTFPAVLQS | |
| | SGLYSLSSVVTVPSSSLGTQTYICNVNHKPSNTKVDKKVEP | |
| | KSCDKTHTCPPCPAPEAAGGPSVFLFPPKPKDTLMISRTPEV | |
| | TCVVVDVSHEDPEVKFNWYVDGVEVHNAKTKPREEQYNS | |
| | TYRVVSVLTVLHQDWLNGKEYKCKVSNKALRAPIEKTISK | |
| | AKGQPREPQVYTLPPCRDELTKNQVSLWCLVKGFYPSDIA | |
| | VEWESNGQPENNYKTTPPVLDSDGSFFLYSKLTVDKSRWQ | |
| | QGNVFSCSVMHEALHNHYTQKSLSLSP | |

Anti-P329G (VH3VL1) x anti-CD3 (Clone 22) 2 + 1 TCB P329R LALA Fc

| | | |
|---|---|---|
| LC1 | QAVVTQEPSLTVSPGGTVTLTCRSSTGAVTTSNYANWVQE | 89 |
| PG | KPDHLFTGLIGGTNKRAPGTPARFSGSLLGGKAALTLSGAQ | |
| | PEDEAEYYCALWYSNHWVFGGGTKLTVLGQPKAAPSVTL | |
| | FPPSSKKLQANKATLVCLISDFYPGAVTVAWKADSSPVKA | |
| | GVETTTPSKQSNNKYAASSYLSLTPEQWKSHRSYSCQVTH | |
| | EGSTVEKTVAPTECS | |
| LC2 | EVQLLESGGGLVQPGGSLRLSCAASGFQFSSYAMNWVRQA | 180 |
| CD3 | PGKGLEWVSRIRSKYNNYATYYADSVKGRFTISRDDSKNT | |
| (crossed) | LYLQMNSLRAEDTAVYYCVRHTTFPSSYVSYYGYWGQGT | |
| | LVTVSSASVAAPSVFIFPPSDEQLKSGTASVVCLLNNFYPRE | |
| | AKVQWKVDNALQSGNSQESVTEQDSKDSTYSLSSTLTLSK | |
| | ADYEKHKVYACEVTHQGLSSPVTKSFNRGEC | |
| HC1 | EVQLVESGGGLVQPGGSLRLSCAASGFTFSRYWMNWVRQ | 178 |
| PG | APGKGLEWVGEITPDSSTINYAPSLKGRFTISRDNAKNSLYL | |
| hole | QMNSLRAEDTAVYYCARPYDYGAWFASWGQGTLVTVSS | |
| | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDKTHTCPPCPAPEAAGGPSV | |
| | FLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALRAPIEKTISKAKGQPREPQVCTLPPSRDELTK | |
| | NQVSLSCAVKGFYPSDIAVEWESNGQPENNYKTTPPVLDS | |
| | DGSFFLVSKLTVDKSRWQQGNVFSCSVMHEALHNHYTQK | |
| | SLSLSP | |
| HC2 | EVQLVESGGGLVQPGGSLRLSCAASGFTFSRYWMNWVRQ | 179 |
| PG | APGKGLEWVGEITPDSSTINYAPSLKGRFTISRDNAKNSLYL | |
| CD3 | QMNSLRAEDTAVYYCARPYDYGAWFASWGQGTLVTVSS | |
| knob | ASTKGPSVFPLAPSSKSTSGGTAALGCLVEDYFPEPVTVSW | |
| | NSGALTSGVHTFPAVLQSSGLYSLSSVVTVPSSSLGTQTYIC | |
| | NVNHKPSNTKVDEKVEPKSCDGGGGSGGGGGQAVVTQEP | |
| | SLTVSPGGTVTLTCGSSTGAVTTSNYANWVQEKPGQAFRG | |
| | LIGGTNKRAPGTPARFSGSLLGGKAALTLSGAQPEDEAEYY | |
| | CALWYSNLWVFGGGTKLTVLSSASTKGPSVFPLAPSSKSTS | |
| | GGTAALGCLVKDYFPEPVTVSWNSGALTSGVHTFPAVLQS | |
| | SGLYSLSSVVTVPSSSLGTQTYICNVNHKPSNTKVDKKVEP | |
| | KSCDKTHTCPPCPAPEAAGGPSVFLFPPKPKDTLMISRTPEV | |
| | TCVVVDVSHEDPEVKFNWYVDGVEVHNAKTKPREEQYNS | |
| | TYRVVSVLTVLHQDWLNGKEYKCKVSNKALRAPIEKTISK | |
| | AKGQPREPQVYTLPPCRDELTKNQVSLWCLVKGFYPSDIA | |
| | VEWESNGQPENNYKTTPPVLDSDGSFFLYSKLTVDKSRWQ | |
| | QGNVFSCSVMHEALHNHYTQKSLSLSP | |

### EXAMPLES

The following are examples of methods and compositions of the invention. It is understood that various other embodiments may be practiced, given the general description provided above.

### General Methods

Recombinant DNA Techniques

Standard methods were used to manipulate DNA as described in Sambrook et al., Molecular cloning: A laboratory manual; Cold Spring Harbor Laboratory Press, Cold Spring Harbor, New York, 1989. The molecular biological reagents were used according to the manufacturers' instructions. General information regarding the nucleotide sequences of human immunoglobulins light and heavy chains is given in: Kabat, E. A. et al., (1991) Sequences of Proteins of Immunological Interest, 5^{th} ed., NIH Publication No. 91-3242.

DNA Sequencing

DNA sequences were determined by double strand sequencing.

Gene Synthesis

Desired gene segments where required were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning /sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

Production of IgG-Like Proteins in HEK293 EBNA or CHO EBNA Cells

Antibodies and bispecific antibodies were generated by transient transfection of HEK293 EBNA cells or CHO EBNA cells. Cells were centrifuged and, medium was replaced by pre-warmed CD CHO medium (Thermo Fisher, Cat No 10743029). Expression vectors were mixed in CD CHO medium, PEI (Polyethylenimine, Polysciences, Inc, Cat No 23966-1) was added, the solution vortexed and incubated for 10 minutes at room temperature. Afterwards, cells (2 Mio/ml) were mixed with the vector/PEI solution, transferred to a flask and incubated for 3 hours at 37° C. in a shaking incubator with a 5% C02 atmosphere. After the incubation, Excell medium with supplements (80% of total volume) was added (W. Zhou and A. Kantardjieff, Mammalian Cell Cultures for Biologics Manufacturing, DOI: 10.1007/978-3-642-54050-9; 2014). One day after transfection, supplements (Feed, 12% of total volume) were added. Cell supernatants were harvested after 7 days by centrifugation and subsequent filtration (0.2 μm filter), and proteins were purified from the harvested supernatant by standard methods as indicated below.

Production of IgG-Like Proteins in CHO K1 Cells

Alternatively, the antibodies and bispecific antibodies described herein were prepared by Evitria using their proprietary vector system with conventional (non-PCR based) cloning techniques and using suspension-adapted CHO K1 cells (originally received from ATCC and adapted to serum-free growth in suspension culture at Evitria). For the production, Evitria used its proprietary, animal-component free and serum-free media (eviGrow and eviMake2) and its proprietary transfection reagent (eviFect). Supernatant was harvested by centrifugation and subsequent filtration (0.2 μm filter) and, proteins were purified from the harvested supernatant by standard methods.

Purification of IgG-Like Proteins

Proteins were purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation (Millipore Amicon® ULTRA-15 (Art.Nr.: UFC903096), and aggregated protein was separated from monomeric protein by size exclusion chromatography in 20 mM histidine, 140 mM sodium chloride, pH 6.0.

Analytics of IgG-Like Proteins

The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII or LabChip GX Touch (Perkin Elmer) (Perkin Elmer). Determination of the aggregate content was performed by HPLC chromatography at 25° C. using analytical size-exclusion column (TSKgel G3000 SW XL or UP-SW3000) equilibrated in running buffer (200 mM KH2PO4, 250 mM KCl pH 6.2, 0.02% NaN3).

### Example 1

### Generation and Characterization of Humanized Anti-P329G Antibodies

Parental and humanized anti-P329G antibodies were produced in HEK cells and purified by ProteinA affinity chromatography and size exclusion chromatography. All antibodies were purified in good quality (Table 2).

TABLE 2
Biochemical analysis of anti-P329G antibodies. Monomer content
determined by analytical size exclusion chromatography. Purity
determined by non-reducing SDS capillary electrophoresis.

| Molecule | Monomer [% ] | Purity [ %] |
|---|---|---|
| Anti-P329G (M-1.7.24) huIgG1 | 100 | 85 |
| Anti-P329G (VH1VL1) huIgG1 | 100 | 97 |
| Anti-P329G (VH2VL1) huIgG1 | 100 | 87 |
| Anti-P329G (VH3VL1) huIgG1 | 100 | 97 |

Binding of Parental and Six Humanization Variants of Anti-P329G Binder M-1.7.24 to Human Fc (P329G)
Instrumentation: Biacore T200
Chip: CM5 (#739)
Fc1 to 4: anti-human Fab specific (GE Healthcare 28-9583-25)
Capture: 50 nM IgGs for 40 s (from supernatant)
Analyte: 200 nM human Fc (P329G) (P1AD9000-004) single injection
Running buffer: HBS-EP
T°: 25° C.
Flow: 30 μl/min
Association: 240 sec
Dissociation: 240 sec
Regeneration: 10 mM glycine pH 2.1 for 2×60 sec

SPR experiments were performed on a Biacore T200 with HBS-EP+ as running buffer (0.01 M HEPES pH 7.4, 0.15 M NaCl, 0.005% Surfactant P20 (BR-1006-69, GE Healthcare)). Anti-human Fab specific antibodies (GE Healthcare 28-9583-25) were directly immobilized by amine coupling on a CM5 chip (GE Healthcare). The IgGs were captured from supernatant for 40 s at 50 nM. Two hundred nM of the human Fc (P329G) was passed over the ligand at 30/min for 240 sec to record the association phase. The dissociation phase was monitored for 240 s and triggered by switching from the sample solution to HBS-EP+. The chip surface was regenerated after every cycle using two injections of 10 mM glycine pH 2.1 for 60 sec. Bulk refractive index differences were corrected for by subtracting the response obtained on the reference flow cell 1. The single binding curves were fitted in the dissociation phase to obtain a k_{off} for ease of comparison (Biacore Evaluation software, GE Healthcare).

Affinity of Parental and Three Humanization Variants of Anti-P329G Binder M-1.7.24 to Human Fc (P329G)
Instrumentation: Biacore T200
Chip: CM5 (#772)
Fc1 to 4: anti-human Fab specific (GE Healthcare 28-9583-25)
Capture: 50 nM IgGs for 60 s
Analyte: human Fc (P329G) (P1AD9000-004)
Running buffer: HBS-EP
T°: 25° C.
Dilution: 2-fold dilution in HBS-EP from 0.59 to 37.5 nM
Flow: 30 μl/min
Association: 240 sec
Dissociation: 800 sec
Regeneration: 10 mM glycine pH 2.1 for 2×60 sec

SPR experiments were performed on a Biacore T200 with HBS-EP+ as running buffer (0.01 M HEPES pH 7.4, 0.15 M NaCl, 0.005% Surfactant P20 (BR-1006-69, GE Healthcare)). Anti-human Fab specific antibodies (GE Healthcare 28-9583-25) were directly immobilized by amine coupling on a CM5 chip (GE Healthcare). The IgGs were captured for 60 s at 50 nM. A two-fold dilution serie of the human Fc (P329G) was passed over the ligand at 30 μl/min for 240 sec to record the association phase. The dissociation phase was monitored for 800 s and triggered by switching from the sample solution to HBS-EP+. The chip surface was regenerated after every cycle using two injections of 10 mM glycine pH 2.1 for 60 sec. Bulk refractive index differences were corrected for by subtracting the response obtained on the reference flow cell 1. The affinity constants were derived from the kinetic rate constants by fitting to a 1:1 Langmuir binding using the Biaeval software (GE Healthcare). The measure was performed in triplicate with independent dilution series.

Following samples were analyzed for binding to human Fc (P329G) (Table 3).

TABLE 3
Description of the samples analyzed
for binding to human Fc (P329G).

| Binder | TAPIR ID | Format |
|---|---|---|
| Anti-P329G (M-1.7.24) | P1AE9963 | IgG, supernatant/purified |
| (parental) | | |
| Anti-P329G (VH3VL1) | P1AE9957 | IgG, supernatant/purified |
| Anti-P329G (VH1VL1) | P1AE9955 | IgG, supernatant/purified |
| Anti-P329G (VH2VL1) | P1AE9956 | IgG, supernatant/purified |
| Anti-P329G (VH4VL1) | P1AE9958 | IgG, supernatant |
| Anti-P329G (VH1VL2) | P1AE9959 | IgG, supernatant |
| Anti-P329G (VH1VL3) | P1AE9960 | IgG, supernatant |
| human Fc (P329G) | P1AD9000-004 | Antigen used as analyte |

Human Fc (P329G) was prepared by plasmin digestion of a human IgG1 followed by affinity purification by ProteinA and size exclusion chromatography.

Binding of Parental and Six Humanization Variants of Anti-P329G Binder M-1.7.24 to Human Fc (P329G )

The dissociation phase was fitted to a single curve to help characterize the off-rate. The ratio between binding to capture response level was calculated. (Table 4).

TABLE 4
Binding assessment of six humanization variants
for binding to human Fc (P329G).

| | | | Ratio | |
|---|---|---|---|---|
| | | | binding/ | |
| Binder | TAPIR ID | kd (1/s) | capture | Binding |
| Anti-P329G (M-1.7.24) | P1AE9963-001 | 5.73E−03 | 20 | parental |
| (parental) | | | | |
| Anti-P329G (VH3VL1) | P1AE9957-001 | 5.49E−03 | 20 | as |
| | | | | parental |
| Anti-P329G (VH1VL1) | P1AE9955-001 | 3.88E−03 | 20 | as |
| | | | | parental |
| Anti-P329G (VH2VL1) | P1AE9956-001 | 2.79E−03 | 23 | as |
| | | | | parental |
| Anti-P329G (VH4VL1) | P1AE9958-001 | 1.11E−02 | 19 | reduced |
| Anti-P329G (VH1VL2) | P1AE9959-001 | 7.86E−03 | 10 | reduced |
| Anti-P329G (VH1VL3) | P1AE9960-001 | 1.29E−01 | 3 | reduced |

Affinity of Parental and Three Humanization Variants of Anti-P329G Binder M-1.7.24 to Human F P329G)

Three humanization variants with binding pattern similar to parental were assessed in more details. The kinetic constants for a 1:1 Langmuir binding are summarized in Table 5.

TABLE 5
Kinetic constants (1:1 Langmuir binding). Average and standard
deviation (in parenthesis) of independent triplicate (independent
dilutions series within the same run).

| | | | | | Rmax |
|---|---|---|---|---|---|
| Binder | TAPIR ID | ka (1/Ms) | kd (1/s) | KD (M) | (RU) |
| Anti-P329G | P1AE9963- | 5.03E+05 | 1.58E−03 | 3.17E−09 | 44 |
| (M-1.7.24) | 003 | (4.75E+04) | (3.8E−05) | (3.7E−10) | (2) |
| (parental) | | | | | |
| Anti-P329G | P1AE9957- | 2.74E+05 | 1.44E−03 | 5.27E−09 | 55 |
| (VH3VL1) | 003 | (5.51E+03) | (7.51E−05) | (3.3E−10) | (3) |
| Anti-P329G | P1AE9955- | 2.83E+05 | 1.20E−03 | 4.24E−09 | 48 |
| (VH1VL1) | 003 | (7.94E+03) | (4.73E−05) | (2.5E−10) | (2) |
| Anti-P329G | P1AE9956- | 2.53E+05 | 1.22E−03 | 4.81E−09 | 54 |
| (VH2VL1) | 003 | (3.79E+03) | (3.61E−05) | (2.1E−10) | (5) |

Conclusion

Six humanization variants were generated. Three of them (VH4VL1, VH1VL2, VH1VL3) showed decreased binding to human Fe (P329G) compared to parental M-1.7.24. The other three humanization variants (VH1VL1, VH2VL1, VH3VL1) have a binding kinetic very similar to the parental binder and did not lose affinity through humanization.

### Example 2

### P329x Variants for Abrogation of FcγR Binding to Human IgG1 Fc

Design of Fc Variants with Minimal Effector Function that are not Recognized by an Antibody Binding to the P329G Fc Framework.

The use of the antibody specific for the P329G mutation (doi:10.1093/protein/gzz027.) within the IgG1 Fc framework as a universal antibody required engineering of the Fc to abrogate binding to Fc gamma receptors, while maintaining similar serum persistence. The example describes the modification of the Proline 329 surrounding of human IgG1 to design a silent Fc, which is not recognized by the P329G specific antibody.

Two parameters were considered: a) the sidechain of the residue at position 329 should not be able to form the so-called proline sandwich between two conserved tryptophan sidechains within the Fc gamma receptors (doi: 10.1093/protein/gzw040.). b) the mutation at position 329 should not be recognized by the anti-P329G specific antibody (doi.org/10.1093/protein/gzz027).

The analysis of the structure of the anti-P329G antibody in complex with an Fc (PDB code: 6S5A) revealed that the Gly329 of the Fc (chain H, of 6S5A) is in close contact with at least three residues of the heavy chain variable domain of the anti-P329G antibody (Trp33, Pro100, and Trp106 of chain H. Residue numbering according to the PDB entry). It was therefore concluded that any sidechain bigger than that of a glycine would decrease the antibody binding because of an increased repulsion. In order to avoid novel B-cell epitopes, which could lead to unwanted recognition of this antibody by the host defense, large, surface-exposed, hydrophilic residues, commonly involved in antibody binding, such as lysine, glutamine, and glutamate, were not selected (doi: 10.1073/pnas.0804851105). Despite these considerations, arginine was estimated to be the one with the biggest repulsion potential and was therefore included. Smaller residues such as alanine, leucine, isoleucine (doi: 10.1073/pnas.0804851105) were not included. Four amino acids were selected based on these considerations: alanine, leucine, isoleucine and arginine (listed from the smallest to the largest sidechains). The mutations, termed P329A, P329L, P329I and P329R were introduced in huIgG1 framework.

Preparation of P329x huIgG1 Variants

The variable region of heavy and light chain DNA sequences encoding a binder specific for 4-1BB, were subcloned in frame with either the constant heavy chain or the constant light chain of human IgG1.

In the Fc part, the Proline at 329 was substituted with the following amino acids, Leucine (L), Isoleucine (I), Arginine (R) and Alanine (A). The Pro329 mutations as well as the Leu234Ala and Leu235Ala mutations have been introduced in the constant region of the heavy chain to abrogate binding to Fc gamma receptors. The amino acid sequences of the Fc portion containing P329x muations are SEQ ID NOs:30-33.

The antibodies were produced by transfecting mammalian cells with the corresponding expression vectors in a 1:1 (“heavy chain”: “vector light chain”).

TABLE 6
Biochemical analysis of anti-4-1BB huIgG1 P329x variants

| | Monomer | CE-SDS |
|---|---|---|
| Molecule | [%] | (non-red) |
| anti-4-1BB huIgG1 P329L | 95 | 90 |
| anti-4-1BB huIgG1 P329I | 95 | 89 |
| anti-4-1BB huIgG1 P329R | 96 | 91 |
| anti-4-1BB huIgG1 P329A | 94 | 94 |

Binding of huIgG1 P329x Variants to Recombinant Fcγ Receptors

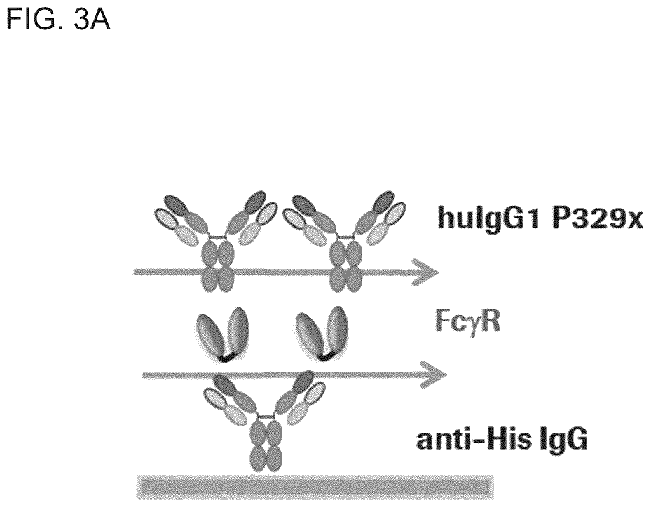

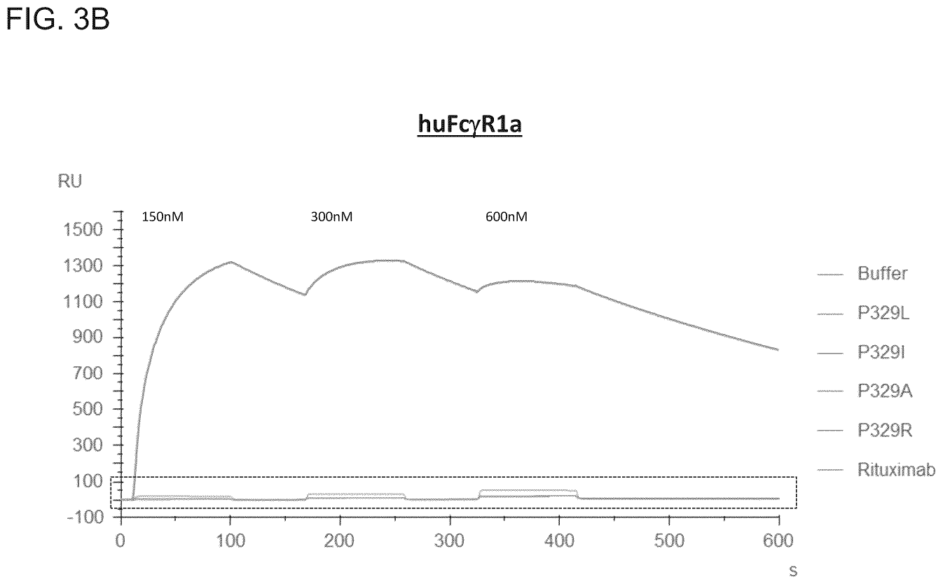

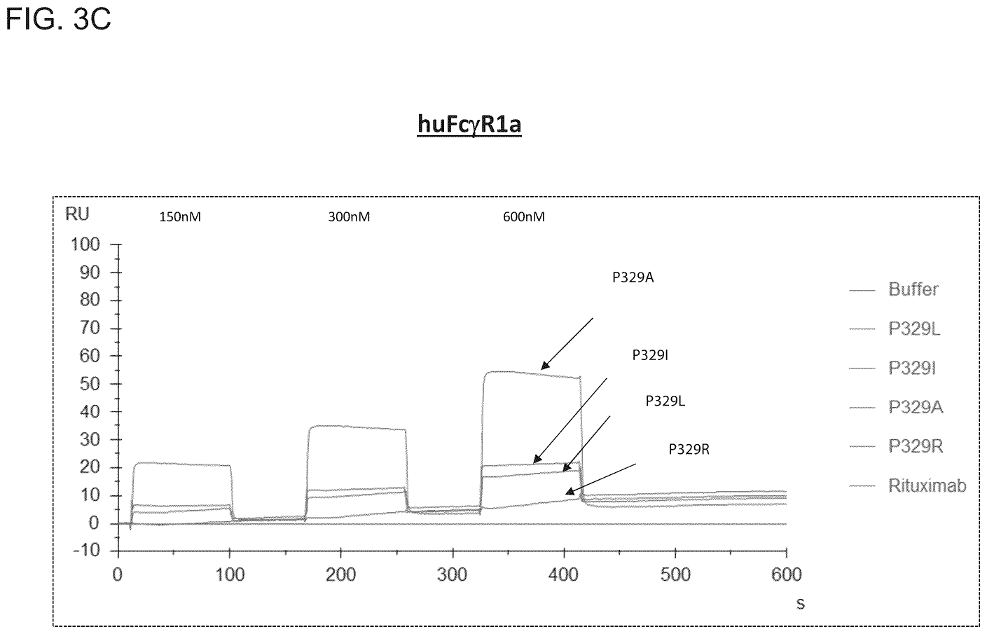

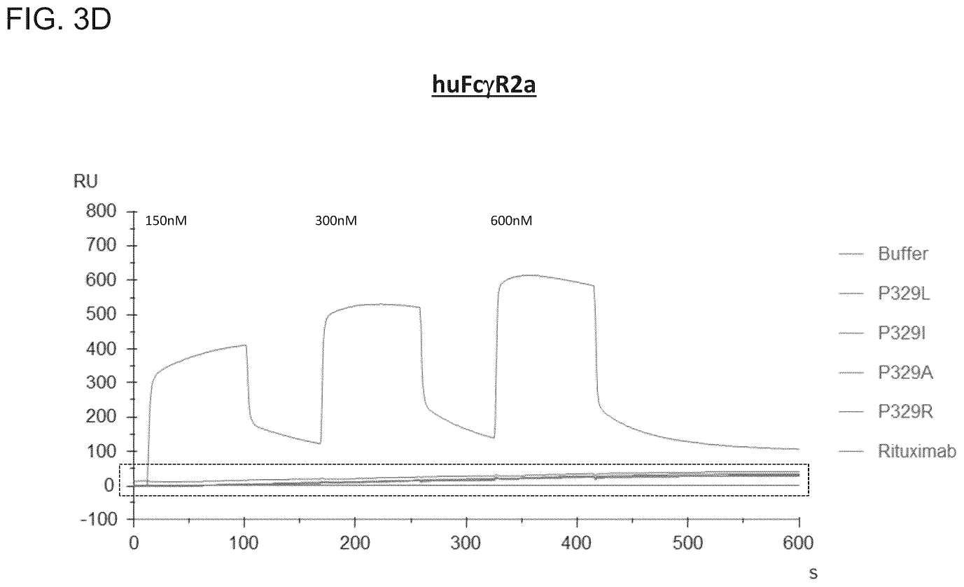

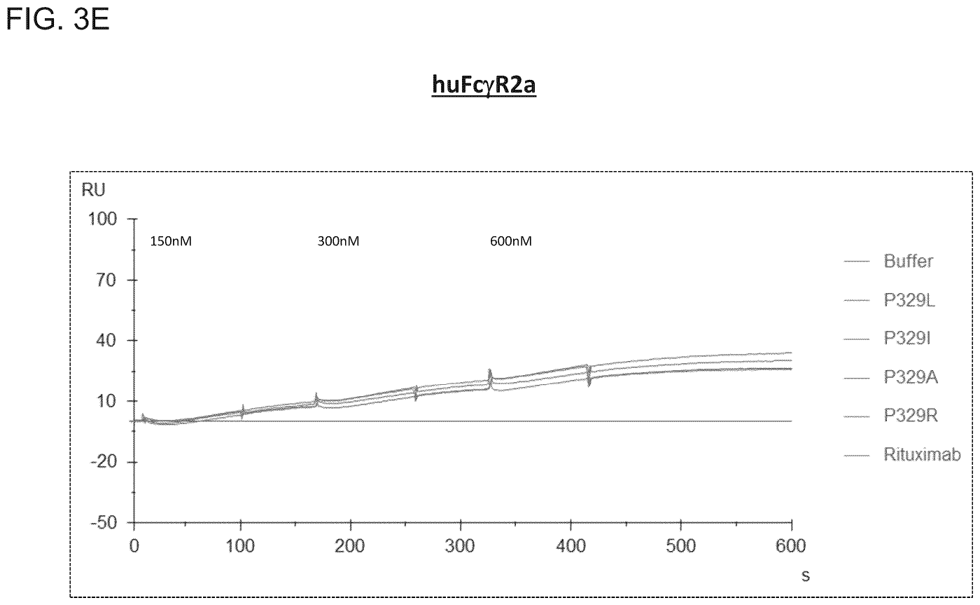

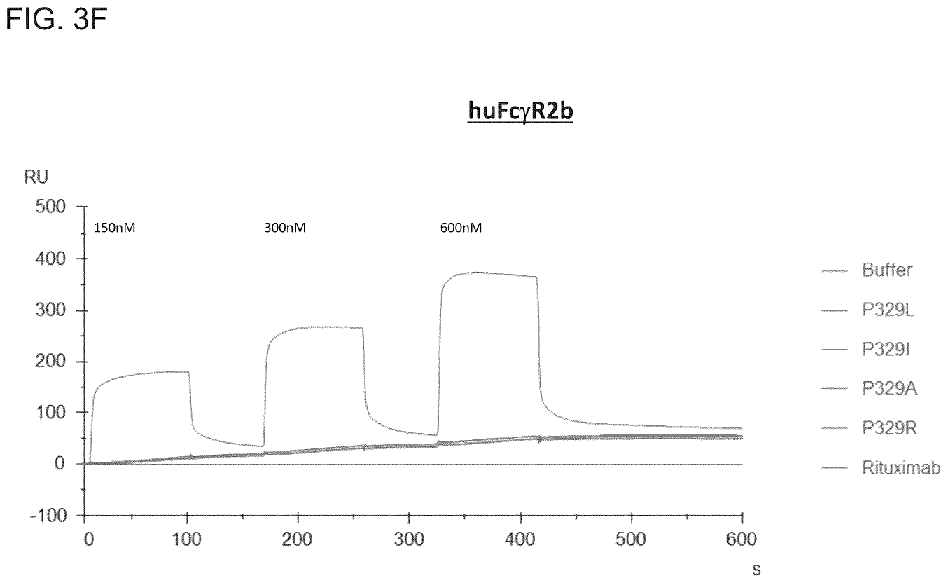

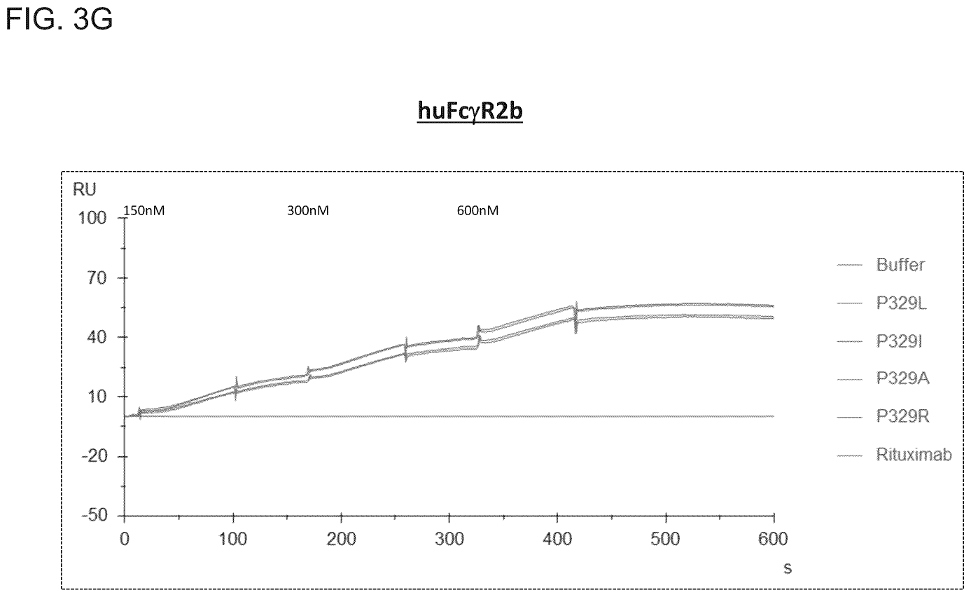

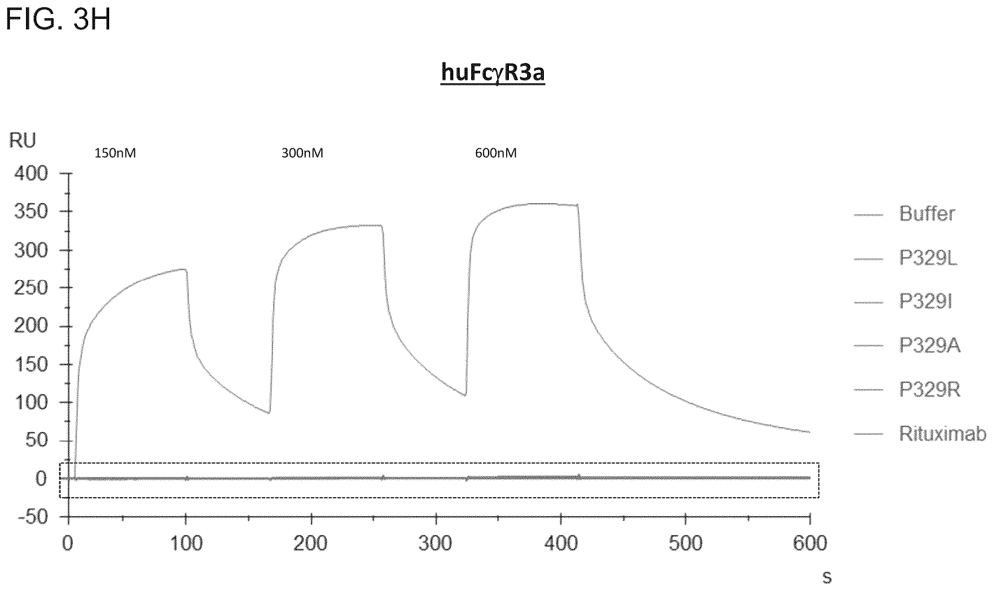

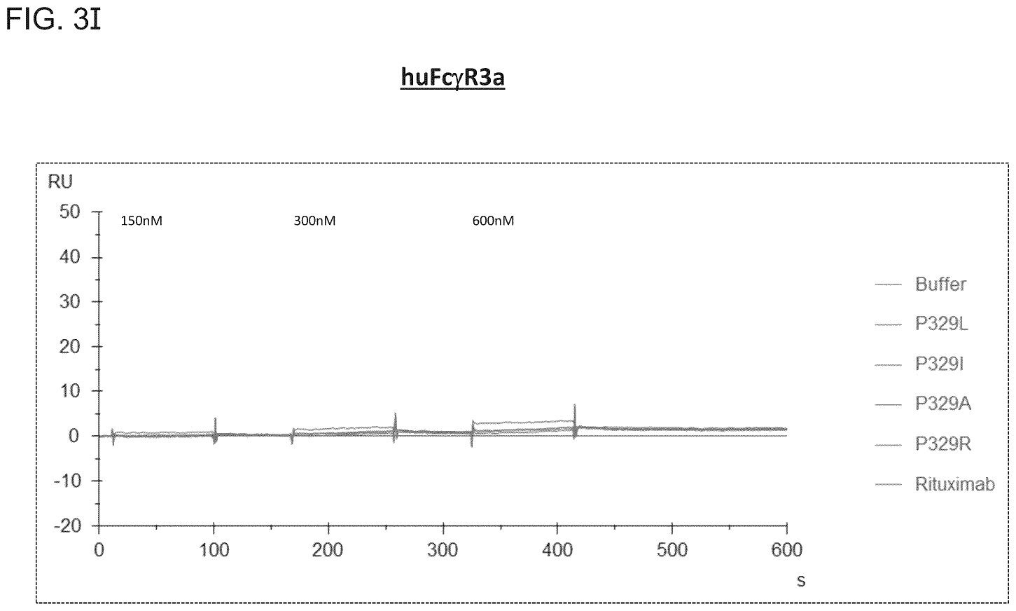

The capacity of binding to recombinant Fe gamma receptors was assessed by surface plasmon resonance (SPR). All SPR experiments were performed on a Biacore T200 at 25° C. with HBS-P+ as running buffer (0.01 M HEPES pH 7.4, 0.30 M NaCl, 3 mM EDTA, 0.005% Surfactant P20, supplied by GE Healthcare ). His-tagged human Fcγ Rs were captured by anti-His antibody coupled to the surface of the CM5 sensor chip. The huIgG1 P329x variants were injected with a flow rate of 20 μl/min in single cycle modus at a concentration of 150, 300 and 600 nM. The dissociation phase was monitored for up to 360 s. The surface was regenerated by 1 min washing with a glycine pH 1.5 solution at a flow rate of 10 μl/min. Bulk refractive index differences were corrected for by subtracting the response obtained from a surface without captured FcγRI. Blank injections are also subtracted (=double referencing). As a positive control, Rituximab (CH B3026) was used in the assay as well since typical IgG1-type binding to FcγRI can be expected. The setup is shown in FIG. 3A.

As can be seen in the sensorgrams of FIG. 3B-FIG. 3E, huIgG1 containing P329L, P329I, P329R and P329A were not able to be bound by human FcγR1a, FcγR2a, FcγR2b and FcγR3a.

Binding of huIgG1 P329x Variants to Anti-P329G Antibody

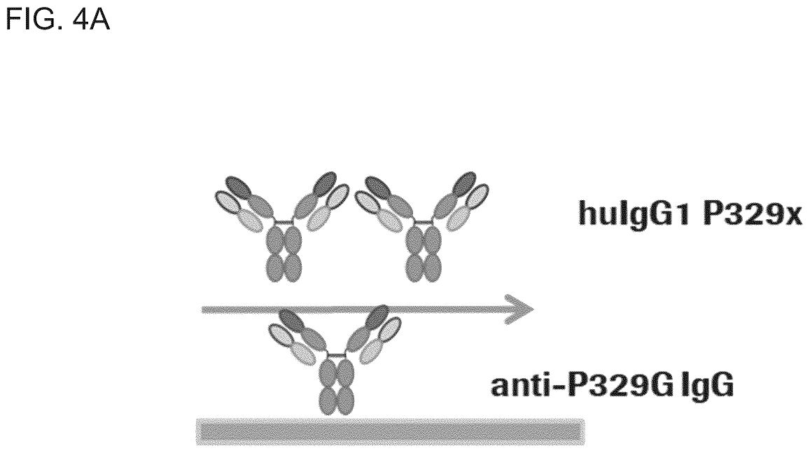

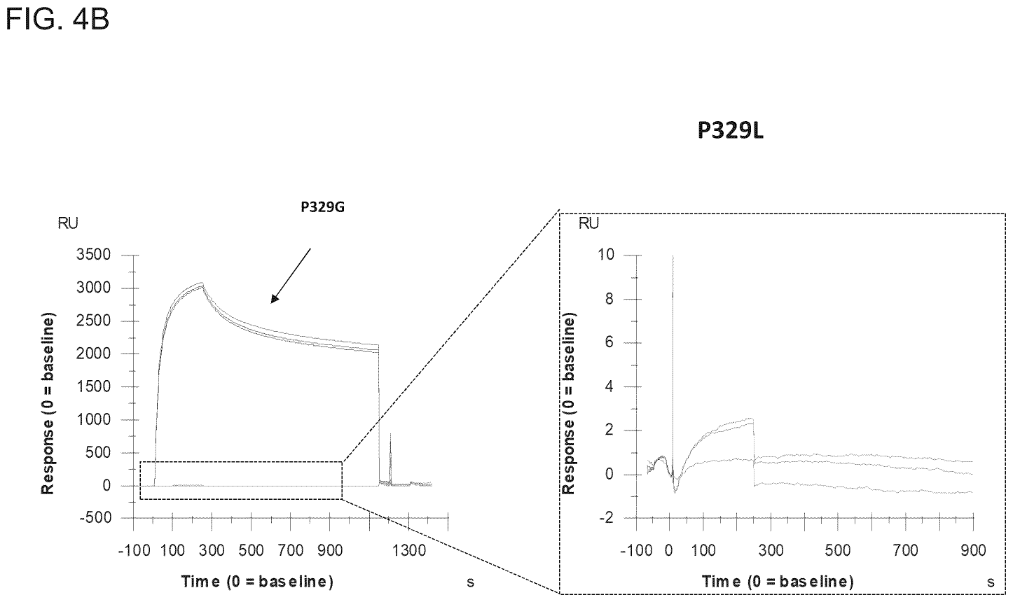

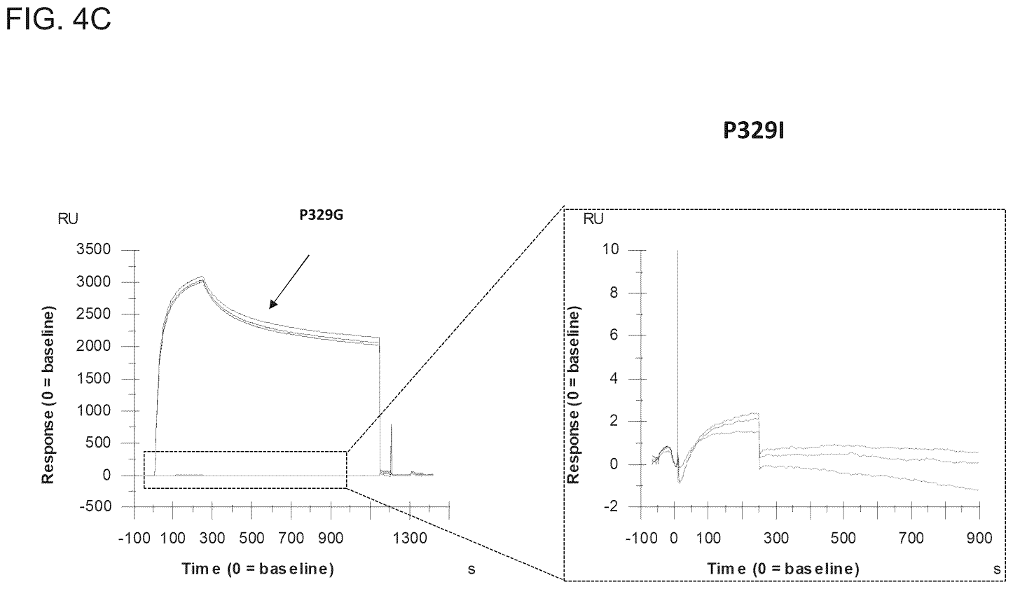

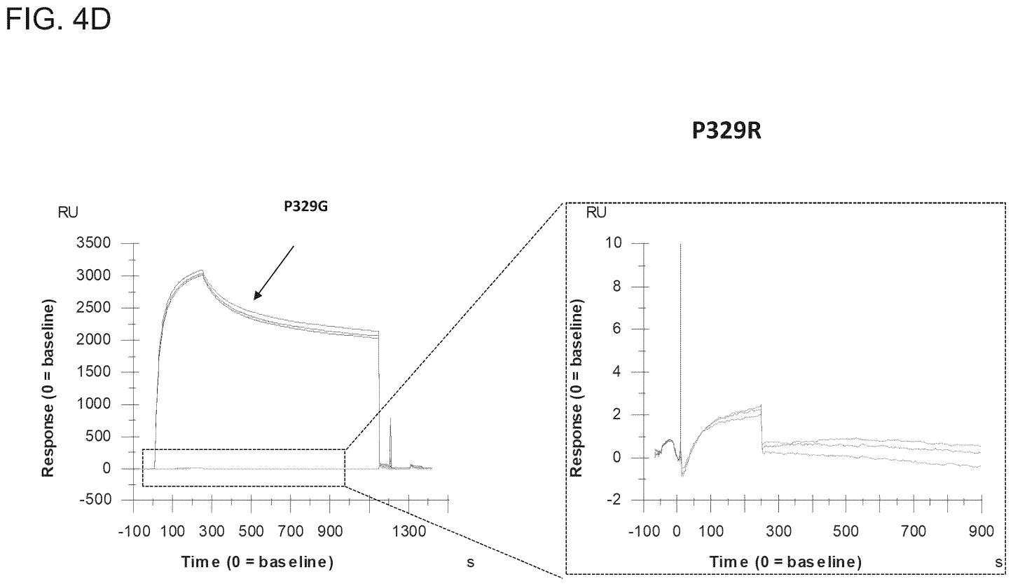

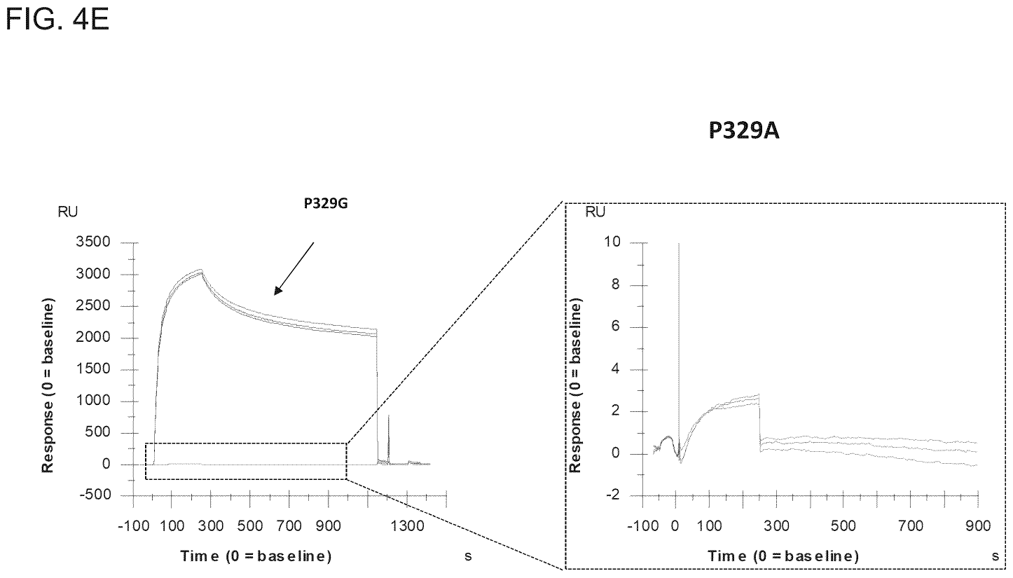

The huIgG1 P329x variants were analysed by surface plasmon resonance (SPR) for their ability to be bound by an anti-P329G antibody (clone M-1.7.24). All SPR experiments were performed on a Biacore T200 at 25° C. with HBS-EP as running buffer (0.01 M HEPES pH 7.4, 0.15 M NaCl, 3 mM EDTA, 0.005% Surfactant P20, Biacore, Freiburg/Germany). The anti-P329G (M-1.7.24) antibody was coupled to the surface of the CM5 sensor chip (immobilization level, approx. 5700 RU). The huIgG1 P329x variants were injected with a flow rate of 30 μl/min at a concentration of 500 nM. The dissociation phase was monitored for up to 600 s. The surface was regenerated by 2 25 min washing with a glycine pH 2.1 solution at a flow rate of 30 μl/min. Bulk refractive index differences were corrected for by subtracting the response obtained from a surface with no antibody immobilized. The setup can be seen in FIG. 4A.

The huIgG1 antibodies with P329L, P329I, P329A or P329R mutations, are not recognized by the anti-P329G binder M-1.7.24, which is specific for a human Fc carrying the P329G mutation (FIG. 4B-FIG. 4E). Similarly to P329G, the P329L/I/A/R mutations can be used to render the human Fe effector-silent. Contrarily to P329G, the P329L/I/A/R mutations are not recognized by the anti-P329G binder.

### Example 3

### Preparation of Optimized Anti-CD3 (Multispecific) Antibodies

All optimized anti-CD3 antibodies (clones P033.078, P035.093, P035.064, P021.045, P004.042) were generated by phage display selection campaigns using libraries derived from a previously described (see e.g. WO 2014/131712, incorporated herein by reference) CD3 binder, termed “CD3_{orig},” herein and comprising the VH and VL sequences of SEQ ID NOs 47 and 56, respectively.

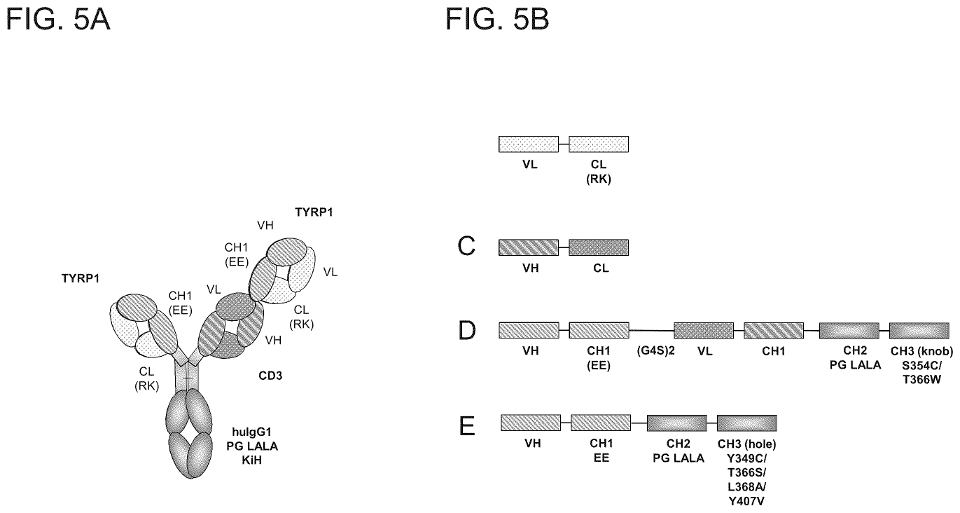

In these libraries, positions N97 and N100 (Kabat numbering) located in the CDR3 region of the heavy chain were either silenced or removed. For direct comparison, all molecules were converted into T-cell bispecific antibody (TCB) format, as depicted in FIG. 5A, using an anti-TYRP1 antibody as exemplary target cell antigen binding moiety (SEQ ID NOs 57-64).

The variable region of heavy and light chain DNA sequences were subcloned in frame with either the constant heavy chain or the constant light chain pre-inserted into the respective recipient mammalian expression vectors as shown in FIG. 5B-FIG. 5E.

Sequences of the optimized anti-CD3 antibodies are given in the SEQ ID NOs indicated in Table 7.

TABLE 7
Sequences of optimized anti-CD3 antibodies generated in the present Examples.

| Clone | HCDR1 | HCDR2 | HCDR3 | VH | LCDR1 | LCDR2 | LCDR3 | VL |
|---|---|---|---|---|---|---|---|---|
| P033.078 | 35 | 38 | 42 | 48 | 53 | 54 | 55 | 56 |
| P035.093 | 35 | 37 | 43 | 49 | 53 | 54 | 55 | 56 |
| P035.064 | 36 | 39 | 44 | 50 | 53 | 54 | 55 | 56 |
| P021.045 | 35 | 37 | 45 | 51 | 53 | 54 | 55 | 56 |
| P004.042 | 36 | 40 | 46 | 52 | 53 | 54 | 55 | 56 |
| CD3_{orig} | 34 | 37 | 41 | 47 | 53 | 54 | 55 | 56 |

To improve correct pairing of the light chains with the corresponding heavy chains, mutations were introduced in the human CL (E123R, Q124K) and the human CH1 (K147E, K213E) of the TYRP1 binding Fab molecule.

For correct pairing of the heavy chains (formation of a heterodimeric molecule), knob-into-hole mutations were introduced in the constant region of the antibody heavy chains (T366W/S354C and T366S/L368A/Y407V/Y349C, respectively).

Furthermore, the P329G, L234A and L235A mutations were introduced in the constant region of the antibody heavy chains to abrogate binding to Fcγ receptors.

Full sequences of the prepared TCB molecules are given in SEQ ID NOs 65, 66, 67 and 69 (P033.078), SEQ ID NOs 65, 66, 67 and 70 (P035.093), SEQ ID NOs 65, 66, 67 and 71 (P035.064), SEQ ID NOs 65, 66, 67 and 72 (P021.045), SEQ ID NOs 65, 66, 67 and 73 (P004.042).

A corresponding molecule comprising CD3_{orig} as CD3 binder was also prepared.

The TCBs were prepared by Evitria (Switzerland) using their proprietary vector system with conventional (non-PCR based) cloning techniques and using suspension-adapted CHO K1 cells (originally received from ATCC and adapted to serum-free growth in suspension culture at Evitria). For the production, Evitria used its proprietary, animal-component free and serum-free media (eviGrow and eviMake2) and its proprietary transfection reagent (eviFect). The cells were transfected with the corresponding expression vectors in a 1:1:2:1 (“vector knob heavy chain”:“vector hole heavy chain”:“vector CD3 light chain”:“vector TYRP1 light chain”). Supernatant was harvested by centrifugation and subsequent filtration (0.2 μm filter) and, proteins were purified from the harvested supernatant by standard methods.

In brief, Fc containing proteins were purified from filtered cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation (Millipore Amicon® ULTRA-15, #UFC903096), and aggregated protein was separated from monomeric protein by size exclusion chromatography in 20 mM histidine, 140 mM sodium chloride, pH 6.0.

The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII (Perkin Elmer). Determination of the aggregate content was performed by HPLC chromatography at 25° C. using analytical size-exclusion column (TSKgel G3000 SW XL or UP-SW3000) equilibrated in running buffer (25 mM K₂HPO₄, 125 mM NaCl, 200 mM L-arginine monohydrocloride, pH 6.7 or 200 mM KH₂PO₄, 250 mM KCl pH 6.2, respectively).

Results from the biochemical and biophysical analysis of the prepared TCB molecules are given in Table 8.

All TCB molecules could be produced in good quality.

TABLE 8
Biochemical and biophysical analysis
of anti-CD3 antibodies in TCB format.

| | | analytical size exclusion | | | CE-SDS |
|---|---|---|---|---|---|
| anti-CD3 | yield | chromatography [%] | | | (main peak) |
| antibody | [mg/l] | HMW | monomer | LMW | [%] |
| P033.078 | 32.8 | 0 | 100 | 0 | 100 |
| P035.093 | 26.8 | 0 | 100 | 0 | 100 |
| P035.064 | 46.4 | 0 | 100 | 0 | 100 |
| P021.045 | 25.9 | 0 | 100 | 0 | 100 |
| P004.042 | 28.7 | 0.4 | 99.6 | 0 | 100 |
| CD3_{orig} | 18.7 | 0 | 100 | 0 | 100 |

Determination of Thermal Stability of Optimized Anti-CD3 (Multispecific) Antibodies Thermal stability of the anti-CD3 antibodies prepared in Example 1 (in TCB format) was monitored by Dynamic Light Scattering (DLS) and by monitoring of temperature dependent intrinsic protein fluorescence by applying a temperature ramp using an Optim 2 instrument (Avacta Analytical, UK).

10 μg of filtered protein sample with a protein concentration of 1 mg/ml was applied in duplicate to the Optim 2. The temperature was ramped from 25 to 85° C. at 0.1° C./min, with the ratio of fluorescence intensity at 350 nm/330 nm and scattering intensity at 266 nm being collected.

The results are shown in Table 9. The aggregation temperature (T_{agg}) and the midpoint of the observed temperature induced unfolding transition (Tₘ) of all the optimized CD3 binders produced in Example 1 is comparable or higher than for the previously described CD3 binder CD3_{orig}.

TABLE 9
Thermal stability of anti-CD3 antibodies in TCB format
as measured by dynamic light scattering and change of
temperature dependent intrinsic protein fluorescence.

| anti-CD3 antibody | Tₘ [° C.] | T_{agg} [° C.] |
|---|---|---|
| P033.078 | 57 | 56 |
| P035.093 | 58.5 | 57 |
| P035.064 | 57.5 | 54 |
| P021.045 | 58.5 | 54 |
| P004.042 | 59 | 56 |
| CD3_{orig} | 57 | 54 |

Functional Characterization of Optimized Anti-CD3 (Multispecific) Antibodies by Surface Plasmon Resonance (SPR)

All surface plasmon resonance (SPR) experiments were performed on a Biacore T200 at 25° C. with HBS-EP+ as running buffer (0.01 M HEPES pH 7.4, 0.15 M NaCl, 3 mM EDTA, 0.005% Surfactant P20; Biacore, Freiburg/Germany).

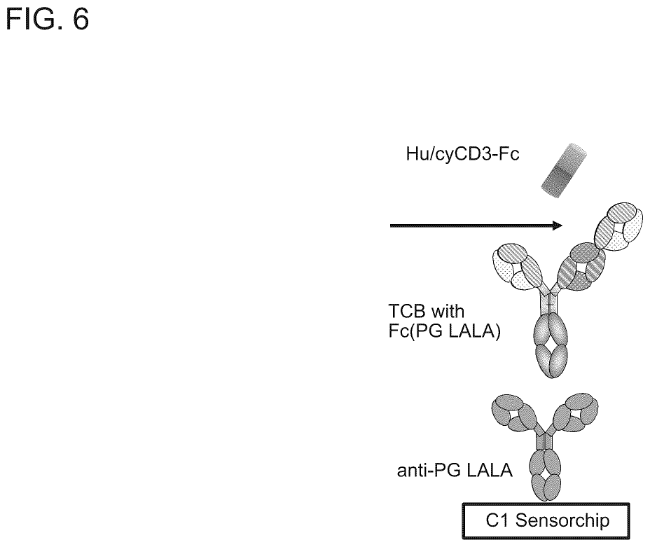

For affinity measurements, TCB molecules were captured on a C1 sensorchip (GE Healthcare) surface with immobilized anti-Fc(P329G) IgG (an antibody that specifically binds human IgG₁ Fc(P329G); “anti-PG antibody”—see WO 2017/072210, incorporated herein by reference). The experimental setup is schematically depicted in FIG. 6. Capture IgG was coupled to the sensorchip surface by direct immobilization of around 400 resonance units (RU) using the standard amine coupling kit (GE Healthcare Life Sciences).

To analyze the interaction to CD3, TCB molecules were captured for 80 s at 25 nM with a flow rate of 10 p/min. Human and cynomolgus CD3c stalk-Fc(knob)-Avi/CD36 stalk-Fc(hole) (CD30/, see SEQ ID NOs 41 and 42 (human) and SEQ ID NOs 43 and 44 (cynomolgus)) were passed at a concentration of 0.122-125 nM with a flow rate of 30 μl/min through the flow cells for 300 s. The dissociation was monitored for 800 s.

Bulk refractive index differences were corrected for by subtracting the response obtained on the reference flow cell. Here, the antigens were flown over a surface with immobilized anti-PG antibody but on which HBS-EP has been injected instead of the TCB molecules.

Kinetic constants were derived using the Biacore T200 Evaluation Software (GE Healthcare Life Sciences), to fit rate equations for 1:1 Langmuir binding by numerical integration. The half-life (t_{1/2}) of the interaction was calculated using the formula t_{1/2}=ln2/k_{off}.

In Table 10 all kinetic parameters of the binding of the optimized anti-CD3 antibodies compared to the previously described binder CD3_{orig} are listed. The optimized anti-CD3 antibodies (in TCB format) are binding to CD3ε/δ with K_{D} values in the in low nM range to high pM range, with K_{D}-values of 600 pM up to 1.54 nM for human CD3ε/δ and 200 pM to 700 pM for cynomolgus CD3F/6. Compared to CD3_{orig} the affinity of the binding to human CD3F/6 of the optimized anti-CD3 antibodies is increased up to 7 to 10 fold as measured under same conditions by SPR.

The half-life of the monovalent binding to human CD3F/6 is with 11.6 min for anti-CD3 antibody clone P033.078 up to 6-fold higher than the binding half-life of CD3_{orig}.

TABLE 10
Affinity of anti-CD3 antibodies (in TCB format)
to human and cynomolgus CD3ε/δ.

| | | Kinetic values at T = 25° C. | | | |
|---|---|---|---|---|---|
| | anti-CD3 | | | | t_{1/2} |
| antigen | antibody | kₒₙ [1/Ms] | k_{off} [1/s] | K_{D} [M] | [min] |
| Human | P033.078 | 1.66E+06 | 9.96E−04 | 6.00E−10 | 11.6 |
| CD3ε/δ | P035.093 | 3.75E+06 | 1.53E−03 | 4.10E−10 | 7.55 |
| | P035.064 | 1.83E+06 | 1.15E−03 | 6.30E−10 | 10 |
| | P021.045 | 3.10E+06 | 1.33E−03 | 4.30E−10 | 8.69 |
| | P004.042 | 1.92E+06 | 2.95E−03 | 1.54E−09 | 3.92 |
| | CD3_{orig} | 5.17E+05 | 3.38E−03 | 6.54E−09 | 3.42 |
| Cynomolgus | P033.078 | 2.20E+06 | 8.02E−04 | 3.70E−10 | 14.4 |
| CD3ε/δ | P035.093 | 4.89E+06 | 1.04E−03 | 2.10E−10 | 11.1 |
| | P035.064 | 2.44E+06 | 9.21E−04 | 3.80E−10 | 12.5 |
| | P021.045 | 4.88E+06 | 9.67E−04 | 2.00E−10 | 11.9 |
| | P004.042 | 3.85E+06 | 2.72E−03 | 7.10E−10 | 4.25 |
| | CD3_{orig} | 1.14E+06 | 2.52E−03 | 2.21E−09 | 4.58 |

Characterization of Optimized Anti-CD3 (Multispecific) Antibodies by Surface Plasmon Resonance (SPR) after Stress

In order to assess the effect of the deamidation site removal and its effect on the stability of the antibodies, the optimized anti-CD3 antibodies (in TCB format) were incubated for 14 days at 37° C., pH 7.4 and at 40° C., pH 6 and further analyzed by SPR for their binding capability to human CD30/. Samples stored at −80° C. pH 6 were used as reference. The reference samples and the samples stressed at 40° C. were in 20 mM His, 140 mM NaCl, pH 6.0, and the samples stressed at 37° C. in PBS, pH 7.4, all at a concentration of 1.0 mg/ml. After the stress period (14 days) samples in PBS were dialyzed back to 20 mM His, 140 mM NaCl, pH 6.0 for further analysis.

All SPR experiments were performed on a Biacore T200 instrument (GE Healthcare) at 25° C. with HBS-P+(10 mM HEPES, 150 mM NaCl pH 7.4, 0.05% Surfactant P20) as running and dilution buffer. Biotinylated human CD3F/6 (see Example 3, SEQ ID NOs 41 and 42) as well as biotinylated anti-huIgG (Capture Select, Thermo Scientific, #7103262100) were immobilized on a Series S Sensor Chip SA (GE Healthcare, #29104992), resulting in surface densities of at least 1000 resonance units (RU). Anti-CD3 antibodies with a concentration of 2 pg/ml were injected for 30 s at a flow rate of 5 μl/min, and dissociation was monitored for 120 s. The surface was regenerated by injecting 10 mM glycine pH 1.5 for 60 s. Bulk refractive index differences were corrected by subtracting blank injections and by subtracting the response obtained from a blank control flow cell. For evaluation, the binding response 5 seconds after injection end was taken. To normalize the binding signal, the CD3 binding was divided by the anti-huIgG response (the signal (RU) obtained upon capture of the CD3 antibody on the immobilized anti-huIgG antibody). The relative binding activity was calculated by referencing each temperature stressed sample to the corresponding, non-stressed sample.

As shown in Table 11, all anti-CD3 antibodies prepared in Example 1 show an improved binding upon stress to CD3ε/δ, as compared to CD3_{orig}.

TABLE 11
Binding activity of anti-CD3 antibodies (in
TCB format) to human CD3ε/δ after
incubation at pH 6/40° C. or pH 7.4/37° C. for 2 weeks.

binding activity [%]

| anti-CD3 | 2 weeks at | 2 weeks at |
|---|---|---|
| antibody | pH 6.0/40° C. | pH 7.4/37° C. |
| P033.078 | 99 | 99 |
| P035.093 | 97 | 95 |
| P035.064 | 97 | 96 |
| P021.045 | 99 | 95 |
| P004.042 | 98 | 98 |
| CD3_{orig} | 95 | 65 |

Jurkat NFAT Reporter Cell Assay with Optimized Anti-CD3 (Multispecific) Antibodies

The (TYRP1-targeted) TCBs containing the optimized anti-CD3 antibodies were tested in the Jurkat NFAT reporter cell assay in the presence of CHO-K1 TYRP1 clone 76 (cells were generated by stable transduction of CHO-K1 cells) as target cells. Jurkat NFAT reporter cells (Promega) were cultured in RPMI 1640 (Gibco) containing 10% FBS, 2 g/l glucose (Sigma), 2 g/l NaHCO₃ (Sigma), 25 mM HEPES (Gibco), 1% GlutaMax (Gibco), 1×NEAA (Sigma), 1% SoPyr (Sigma) (Jurkat NFAT medium) at 0.1-0.5 mio cells/ml. CHO-K1 TYRP1 clone 76 cells were cultured in DMEM/F12+GlutaMAX (1×) (Gibco) containing 10% FBS and 6 μg/ml Puromycin (Invivogen). The assay was performed in Jurkat NFAT medium.

CHO-K1 TYRP1 clone 76 cells were detached using Trypsin (Gibco). The cells were counted and viability was checked. The target cells were re-suspended in assay medium and 10 000 cells were seeded per well in a white flat bottom 384 well plate. Then the TCBs were added at the indicated concentrations. Jurkat NFAT reporter cells were counted, viability was checked and 20 000 cells were seeded per well, corresponding to an effector-to-target (E:T) ratio of 2:1. Also, 2% end-volume of GloSensor cAMP Reagent (E1291, Promega) was added to each well. After the indicated incubation time, luminescence was measured using a Tecan Spark10M device.

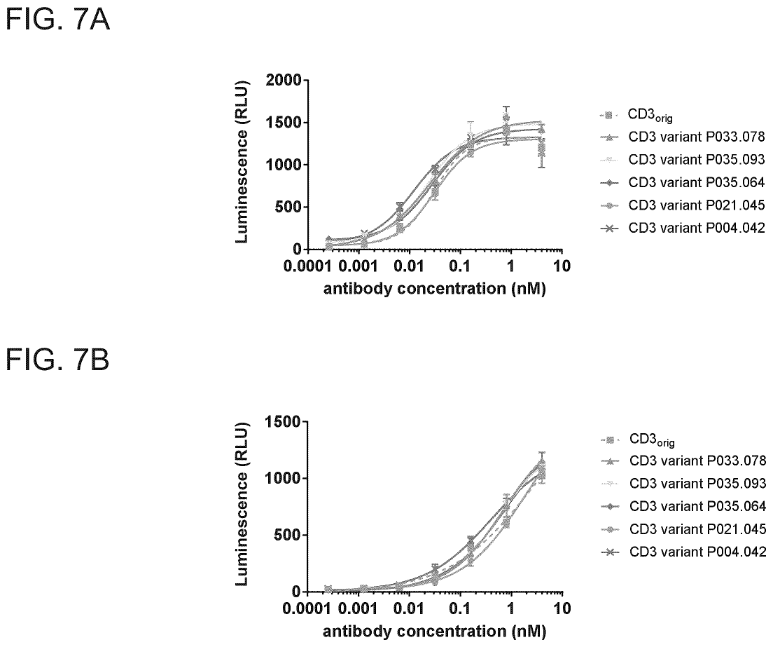

As shown in FIG. 7A-FIG. 7B, the TCBs containing the optimized anti-CD3 antibodies had a similar functional activity on Jurkat NFAT reporter cells as the TCBs containing the parental binder CD3_{orig}. The tested TCBs induced CD3 activation in a concentration dependent manner.

Tumor Cell Killing of Primary Melanoma Cells with Optimized Anti-CD3 (Multispecific) Antibodies

The optimized anti-CD3 antibodies in (TYRP1-targeted) TCB format were tested in a tumor cell killing assay with freshly isolated human PBMCs, co-incubated with the human melanoma cell line M150543 (primary melanoma cell line, obtained from the dermatology cell bank of the University of Zurich). Tumor cell lysis was determined by quantification of LDH released into cell supernatants by apoptotic or necrotic cells after 24 h and 48 h. Activation of CD4 and CD8 T cells was analyzed by upregulation of CD69 and CD25 on both cell subsets after 48 h.

On the day before assay start, target cells (M150543) were detached using Trypsin (Gibco), washed once with PBS and re-suspended at a density of 0.3 mio cells/ml in growth medium (RPMI 1640 (Gibco) containing 10% FBS, 1% GlutaMax (Gibco) and 1% SoPyr (Sigma)). 100 μl of the cell suspension (containing 30 000 cells) were seeded into a 96 well flat bottom plate. The cells were incubated overnight at 37° C. in the incubator.

The next day, PBMCs were isolated from blood of a healthy donor and viability was checked. Medium was removed from plated target cells and 100 μl of assay medium (RPMI 1640 (Gibco) containing 2% FBS and 1% GlutaMax (Gibco)) were added to the wells. Antibodies were diluted in assay medium at indicated concentrations and 50 μl per well were added to the target cells. Assay medium was added to control wells. Isolated PBMCs were re-suspended at a density of 6 mio cells/ml, 50 μl were added per well resulting in 300 000 cells/well (E:T 10:1). For determination of spontaneous LDH release (minimal lysis=0%), PBMCs and target cells only were co-incubated. For determination of maximal LDH release (maximal lysis=100%), only assay medium was added to target cells. Control wells with PBMCs plus TCBs in absence of target cells were used to test the specificity of the TCBs. To determine if CD8 and CD4 T cells get activated in absence of tumor cells expressing the target, expression of CD25 was analyzed after 48 hours.

Few hours before the first LDH measurement, 50 μl of assay medium containing 4% Triton X-100 (Bio-Rad) was added to the wells containing target cells only (resulting in a final concentration of 1% Triton X-100 per well) for maximal LDH release. The assay was incubated in total for 48 h at 37° C. in the incubator. The first LDH measurement was performed 24 h after assay start. For this, the Cytotoxicity Detection Kit (LDH) (Roche/Sigma, #11644793001) was adjusted to room temperature before measurement. The assay plate was centrifuged for 4 min at 420×g and 50 μl of supernatant per well was transferred to a 96 well flat bottom plate for analysis. Then a reaction mixture of 1.25 μl of LDH Catalyst and 56.25 μl of LDH Substrate per well was prepared. 50 μl of the LDH reaction mixture was subsequently added to each well and absorbance was immediately measured using a TECAN Infinite F50 instrument. The measurement was repeated 48 h after assay start.

Afterwards PBMCs were harvested and analyzed by measuring CD25 and CD69 upregulation for activation. In detail, 100 μl of FACS buffer was added to each well and cells were transferred to a 96 well U bottom plate for FACS staining. The plate was centrifuged for 4 min at 400×g, supernatant was removed and cells were washed with 150 μl FACS buffer per well. The plate was again centrifuged for 4 min at 400×g and supernatant was removed. Subsequently 30 μl per well of the antibody mix containing CD4 APC (clone RPA-T4, BioLegend), CD8 FITC (clone SKi, BioLegend), CD25 BV421 (clone BC96, BioLegend) and CD69 PE (clone FN50, BioLegend) was added to the cells. The cells were incubated for 30 min in the fridge. Afterwards the cells were washed twice with FACS buffer and re-suspended in 100 μl FACS buffer containing 1% PFA per well. Before the measurement, cells were resuspended in 150 μl FACS buffer. The analysis was performed using a BD LSR Fortessa device.

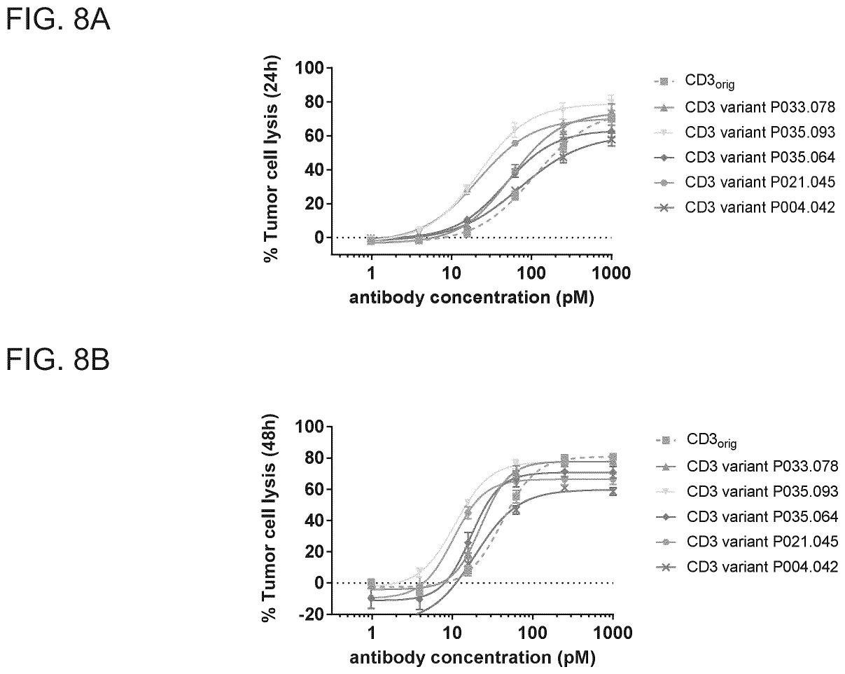

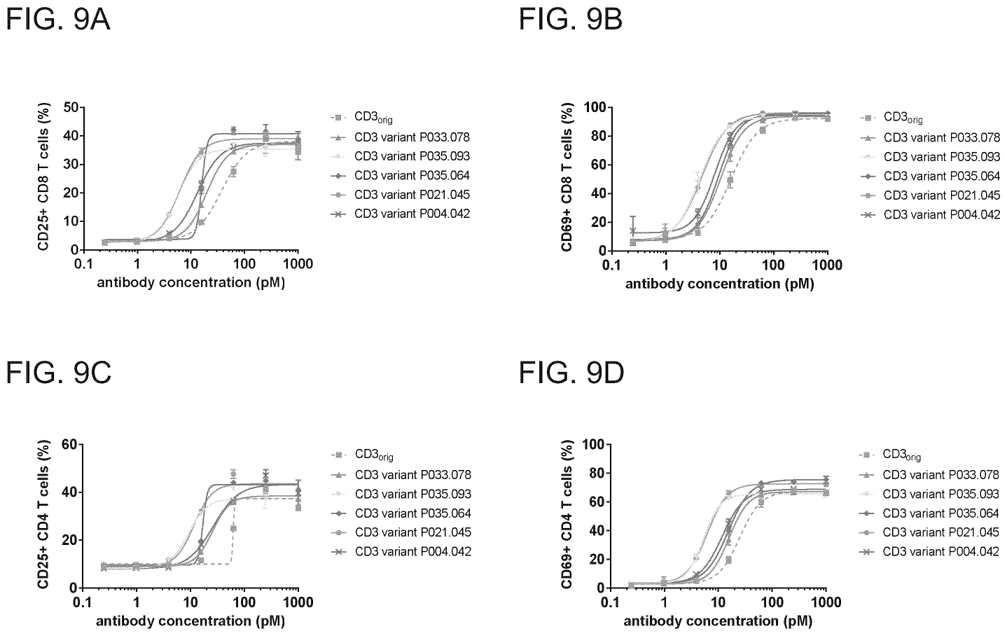

Treatment with TCBs containing the anti-CD3 antibody clone P035.093 and clone P021.045 led to highest tumor cell killing, the clone P033.078 and clone P035.064 resulted in a medium degree of tumor cell killing, followed by clone P004.042 inducing similar tumor cell killing compared to TCBs containing the parental binder CD3_{orig} (FIG. 8A-FIG. 8B). Activation of T cells is highest when treated with TCBs containing the anti-CD3 antibody clone P035.093 and clone P021.045, whereas the TCBs containing the other anti-CD3 antibody clones led to similar T cell activation as to the TCBs containing the parental binder CD3_{orig} (FIG. 9A-FIG. 9D).

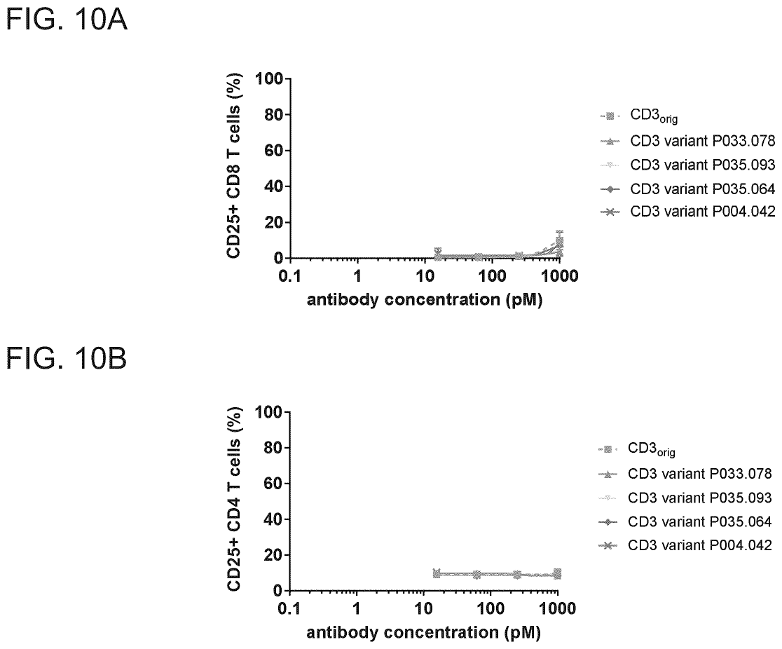

As shown in FIG. 10A-FIG. 10B, the tested TCBs did not induce CD25 upregulation on CD8 and CD4 T cells in absence of tumor target cells. This result shows that the tested CD3 binders depend on crosslinking for example via binding to a tumor cell to induce T cell activation and are not able to induce T cell activation in a monovalent format.

Preparation of Optimized Anti-CD3 Antibodies

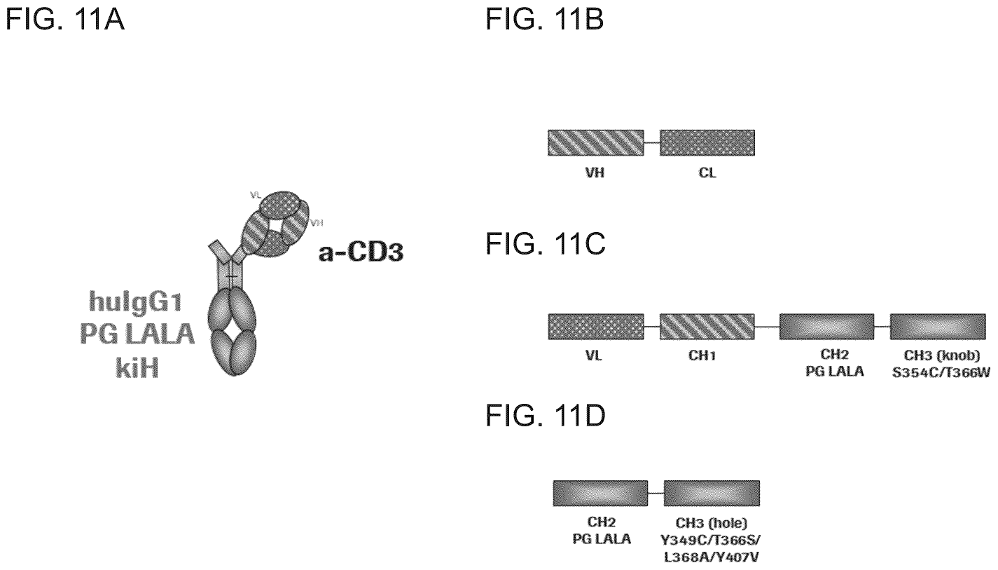

The optimized anti-CD3 antibodies clones P033.078, P035.093, and P004.042 were converted into monovalent human IgG₁ format, with crossed VH and VL domains on the CD3 binding moeity as depicted in FIG. 11A.

The variable region of heavy and light chain DNA sequences were subcloned in frame with either the constant heavy chain or the constant light chain pre-inserted into the respective recipient mammalian expression vectors as shown in FIG. 11B-FIG. 11D.

For correct pairing of the heavy chains (formation of a heterodimeric molecule), knob-into-hole mutations were introduced in the constant region of the antibody heavy chains (T366W/S354C and T366S/L368A/Y407V/Y349C, respectively).

Furthermore, the P329G, L234A and L235A mutations were introduced in the constant region of the antibody heavy chains to abrogate binding to Fcγ receptors.

Corresponding molecules comprising CD3_{orig} as CD3 binder were also prepared.

The monovalent IgG molecules were prepared at Evitria (Switzerland), purified and analysed as described for the TCB molecules in Example 1. For transfection of the cells, the corresponding expression vectors were applied in a 1:1:1 ratio (“vector knob heavy chain”:“vector hole heavy chain”:“vector light chain”).

Results from the biochemical and biophysical analysis of the prepared monovalent IgG molecules are given in Table 12.

All monovalent IgG molecules could be produced in good quality.

TABLE 12
Biochemical and biophysical analysis of anti-
CD3 antibodies in monovalent IgG format.

| | | analytical size exclusion | | | CE-SDS |
|---|---|---|---|---|---|
| anti CD3 | yield | chromatography [%] | | | (main peak) |
| antibody | [mg/l] | HMW | monomer | LMW | [%] |
| P033.078 | 1560 | 0 | 98.9 | 1.1 | 94.6 |
| P035.093 | 2250 | 0 | 98.2 | 1.8 | 92.1 |
| P004.042 | 3360 | 0 | 100 | 0 | 84.5 |
| CD3_{orig} | 1447.5 | 0.9 | 99.1 | 0 | 90.5 |

Determination of Thermal Stability of Optimized Anti-CD3 Antibodies

Thermal stability of the anti-CD3 antibodies in monovalent IgG format was monitored by Dynamic Light Scattering (DLS) and by monitoring of temperature dependent intrinsic protein fluorescence as described before.

The results are shown in Table 13. The aggregation temperature (T_{agg}) and the midpoint of the observed temperature induced unfolding transition (Tₘ) of all the optimized CD3 binders in monovalent IgG format is comparable or higher than for the previously described CD3 binder CD3_{orig}.

TABLE 13
Thermal stability of anti-CD3 antibodies in monovalent IgG
format as measured by dynamic light scattering and change
of temperature dependent intrinsic protein fluorescence.

| anti CD3 antibody | Tₘ [° C.] | T_{agg} [° C.] |
|---|---|---|
| P033.078 | 57.0 | 55.5 |
| P035.093 | 58.0 | 55.5 |
| P004.042 | 58.5 | 56.0 |
| CD3_{orig} | 55 | 53.0 |

Functional Characterization of Optimized Anti-CD3 Antibodies by Surface Plasmon Resonance (SPR)

SPR experiments were performed as described before, with the monovalent IgG molecules as described before.

To analyze the interaction to CD3, IgG molecules were captured for 240 s at 50 nM with a flow rate of 5 μl/min. Human and cynomolgus CD3F stalk-Fc(knob)-Avi/CD36-stalk-Fc(hole) were passed at a concentration of 0.061-250 nM with a flow rate of 30 μl/min through the flow cells for 300 s. The dissociation was monitored for 800 s.

In Table 14 all kinetic parameters of the binding of the optimized anti-CD3 antibodies compared to the previously described binder CD3_{orig} are listed. The optimized anti-CD3 antibodies (in monovalent IgG format) are binding to CD3ε/δ with K_{D} values in the in low nM range to high pM range, with K_{D}-values of 770 pM up to 1.36 nM for human CD3ε/δ and 200 μM to 400 μM for cynomolgus CD3F/6. Compared to CD3_{orig} the affinity of the binding to human CD3F/6 of the optimized anti-CD3 antibodies is increased up to 3.5 to 15-fold as measured under same conditions by SPR.

The half-life of the monovalent binding to human CD3F/6 is with 8.69 min for anti-CD3 antibody clone P033.078 more than 2-fold higher than the binding half-life of CD3_{orig}.

TABLE 14
Affinity of anti-CD3 antibodies (in monovalent IgG
format) to human and cynomolgus CD3ε/δ. Data
obtained from triplicate measurements.

| | | Kinetic values at T = 25° C. | | | |
|---|---|---|---|---|---|
| | anti CD3 | | | | t_{1/2} |
| antigen | antibody | kₒₙ [1/Ms] | k_{off} [1/s] | K_{D} [M] | [min] |
| Human | P033.078 | 1.73E+06 | 1.33E−03 | 7.71E−10 | 8.69 |
| CD3ε/δ | P035.093 | 3.08E+06 | 1.40E−03 | 4.56E−10 | 8.25 |
| | P004.042* | 6.28E+06 | 8.52E−03 | 1.36E−09 | 1.36 |
| | CD3_{orig} | 5.87E+05 | 2.90E−03 | 4.94E−09 | 3.98 |
| Cynomolgus | P033.078 | 2.60E+06 | 1.04E−03 | 4.03E−10 | 11.11 |
| CD3ε/δ | P035.093 | 4.38E+06 | 9.81E−04 | 2.24E−10 | 11.78 |
| | P004.042* | 1.85E+07 | 8.09E−03 | 4.39E−10 | 1.43 |
| | CD3_{orig} | 1.20E+06 | 2.45E−03 | 2.03E−09 | 4.72 |

*kinetic and affinity values may not be fully reliable, due to bad fit quality

### Example 4

### Immune Activating Fc Binding Molecules Comprising an Activating T Cell Antigen Binder

The following molecules were prepared in this example by transfection in mammalian cells and purification by ProteinA affinity chromatography and size exclusion chromatography. Anti-P329G (M-1.7.24)×CD3 (CH2527) 2+1 TCB with charge modifications (VH/VL exchange in CD3 binder) (format of FIG. 2B, SEQ ID NOs 86, 68, 87, 88).

Anti-P329G (VH3VL1)×CD3 (CH2527) 1+1 TCB with charge modifications (VH/VL exchange in CD3 binder) (format of FIG. 2A, SEQ ID NOs 89, 68, 90, 91)

Anti-P329G (VH3VL1)×CD3 (P035.093) 1+1 TCB with charge modifications (VH/VL exchange in CD3 binder) (format of FIG. 2A, SEQ ID NOs 89, 70, 90, 91).

Anti-P329G (VH3VL1)×CD3 (P035.093) 2+1 TCB with charge modifications (VH/VL exchange in CD3 binder) (format of FIG. 2B, SEQ ID NOs 89, 70, 90, 92).

TABLE 15
Biochemical analysis of anti-P329G T cell bispecifics. Monomer content
determined by analytical size exclusion chromatography. Purity
determined by non-reducing SDS capillary electrophoresis.

| | Monomer | Purity |
|---|---|---|
| Molecule | [%] | [%] |
| Anti-P329G (M-1.7.24) × CD3 (CH2527) 2 + 1 | 94 | 97 |
| TCB | | |
| Anti-P329G (VH3VL1) × CD3 (CH2527) 1 + 1 | 100 | 87 |
| TCB | | |
| Anti-P329G (VH3VL1) × CD3 (P035.093) 1 + 1 | 100 | 98 |
| TCB | | |
| Anti-P329G (VH3VL1) × CD3 (P035.093) 2 + 1 | 100 | 100 |
| TCB | | |

Affinity of Anti-P329G×CD3 TCBs to Human CD3 Epsilon-Delta-Fc
Instrumentation: Biacore T200
Chip: SA (#786)
Fc4: human CD3 epsilon-delta-Fc biotinylated (P1AA6127-015)
Analyte: anti-P329G×CD3 TCBs
Running buffer: HBS-EP
T°: 25° C.
Dilution: 3-fold dilution in HBS-EP from 0.41 to 300 nM
Flow: 30 μl/min
Association: 240 sec
Dissociation: 240 sec
Regeneration: 10 mM glycine pH 1.5 for 30 sec

SPR experiments were performed on a Biacore T200 with HBS-EP+ as running buffer (0.01 M HEPES pH 7.4, 0.15 M NaCl, 0.005% Surfactant P20 (BR-1006-69, GE Healthcare)). Biotinylated human CD3 epsilon-delta-Fc was immobilized on a SA chip (GE Healthcare). Three-fold dilution series of anti-P329G×CD3 TCBs were passed over the ligand at 30 μl/min for 240 sec to record the association phase. The dissociation phase was monitored for 240 s and triggered by switching from the sample solution to HBS-EP+. The chip surface was regenerated after every cycle using one injection of 10 mM glycine pH 1.5 for 30 sec. Bulk refractive index differences were corrected for by subtracting the response obtained on the reference flow cell 1. The affinity constants were derived from the kinetic rate constants by fitting to a 1:1 Langmuir binding using the Biaeval software (GE Healthcare). The measure was performed in triplicate with independent dilution series.

The kinetic constants for a 1:1 Langmuir binding for binding of anti-P329G×CD3 TCBs to immobilized human CD3 epsilon-delta-Fc are summarized in Table 16.

TABLE 16
Kinetic constants (1:1 Langmuir binding) of the interaction between anti-P329G ×
CD3 TCBs and human CD3 epsilon-delta-Fc. Average and standard deviation (in parenthesis)
of independent triplicate (independent dilutions series within the same run).

| Binder | TAPIR ID | ka (1/Ms) | kd (1/s) | KD (M) | Rmax (RU) |
|---|---|---|---|---|---|
| Anti-P329G (VH3VL1) × | P1AF4446-004 | 1.19E+06 | 3.36E−03 | 2.82E−09 | 130 |
| CD3 (CH2527) 1 + 1 TCB | | (1.15E+04) | (1.00E−05) | (4.16E−11) | (2) |
| Anti-P329G (VH3VL1) × | P1AF4447-004 | 7.65E+06 | 1.48E−03 | 1.94E−10 | 131 |
| CD3 (P035.093) 1 + 1 TCB | | (2.46E+05) | (1.00E−05) | (8.02E−12) | (1) |
| Anti-P329G (VH3VL1) × | P1AF4448-004 | 3.38E+06 | 1.15E−03 | 3.41E−10 | 179 |
| CD3 (P035.093) 2 + 1 TCB | | (1.83E+05) | (5.77E−06) | (1.73E−11) | (2) |

Affinity of Anti-P329G×CD3 TCBs to TCB (P329G)

Instrumentation: Biacore T200
Chip: C1 (#784, 785)
Fe 2 (784): Anti-P329G (VH3VL1)×CD3 (CH2527) 1+1 TCB
Fe 3 (784): Anti-P329G (VH3VL1)×CD3 (P035.093) 1+1 TCB
Fe 3 (785): Anti-P329G (VH3VL1)×CD3 (P035.093) 2+1 TCB
Analyte: unrelated TCB with P329G on Fe (P1AE7925-003)
Running buffer: HBS-EP
T°: 25° C.
Dilution: 3-fold dilution in HBS-EP from 0.09 to 600 nM
Flow: 30 μl/min
Association: 240 sec
Dissociation: 800 sec
Regeneration: 10 mM glycine pH 1.5 for 2×60 see

SPR experiments were performed on a Biacore T200 with HBS-EP+ as running buffer (0.01 M HEPES pH 7.4, 0.15 M NaCl, 0.005% Surfactant P20 (BR-1006-69, GE Healthcare)). Anti-P329G×CD3 TCBs were directly immobilized by amine coupling on a C1 chip (GE Healthcare). A three-fold dilution series of an unrelated TCB with P329G on Fe was passed over the ligand at 30 μl/min for 240 see to record the association phase. The dissociation phase was monitored for 800 s and triggered by switching from the sample solution to HBS-EP+. The chip surface was regenerated after every cycle using two injections of 10 mM glycine pH 1.5 for 60 sec. Bulk refractive index differences were corrected for by subtracting the response obtained on the reference flow cell 1. The affinity constants were derived from the kinetic rate constants by fitting to a 1:1 Langmuir binding using the Biaeval software (GE Healthcare). The measure was performed in triplicate with independent dilution series.

The kinetic constants for a 1:1 Langmuir binding for binding of anti-P329G×CD3 TCBs to a construct bearing the P329G mutation on the Fe are summarized in Table 17.

TABLE 17
Kinetic constants (1:1 Langmuir binding) of the interaction of anti-P329G × CD3 TCBs
with a TCB bearing the P329G mutation on the Fc. Average and standard deviation (in parenthesis)
of independent triplicate (independent dilutions series within the same run).

| Binder | TAPIR ID | ka (1/Ms) | kd (1/s) | KD (M) | Rmax (RU) |
|---|---|---|---|---|---|
| Anti-P329G (VH3VL1) × | P1AF4446-004 | 2.56E+05 | 8.50E−03 | 3.32E−08 | 54 |
| CD3 (CH2527) 1 + 1 TCB | | (1.79E+04) | (3.74E−04) | (1.88E−09) | (3) |
| Anti-P329G (VH3VL1) × | P1AF4447-004 | 1.68E+05 | 9.80E−04 | 5.91E−09 | 89 |
| CD3 (P035.093) 1 + 1 TCB | | (1.69E+04) | (4.91E−05) | (8.88E−10) | (3) |
| Anti-P329G (VH3VL1) × | P1AF4448-004 | 1.68E+05 | 1.44E−03 | 8.66E−09 | 64 |
| CD3 (P035.093) 2 + 1 TCB | | (1.82E+04) | (9.26E−05) | (1.39E−09) | (2) |

Simultaneous binding of anti-P329G×CD3 TCBs to human CD3 epsilon-delta-Fc and human Fc (P329G)
Instrumentation: Biacore T200
Chip: SA (#786)
Fe 2, 3, 4: human CD3 epsilon-delta-Fc biotinylated (P1AA6127-015)
Analyte: anti-P329G×CD3 TCBs, followed by human Fe (P329G) (P1AD9000-004)
Running buffer: HBS-EP
T°: 25° C.
Flow: 30 μl/min

SPR experiments were performed on a Biacore T200 with HBS-EP+ as running buffer (0.01 M HEPES pH 7.4, 0.15 M NaCl, 0.005% Surfactant P20 (BR-1006-69, GE Healthcare)). Biotinylated human CD3 epsilon-delta-Fc was immobilized on a streptavidin chip. Fifty nM of anti-P329G×CD3 TCB were injected at 30 μl/min for 30 see, followed by 500 nM of human Fe (P329G) for 60 sec.

Samples

Following samples were analyzed for binding to human CD3 epsilon-delta-Fc and to constructs with human Fe bearing the P329G mutation (table 18).

TABLE 18
Description of the samples analyzed for binding to
human CD3 epsilon-delta-Fc and to human Fc (P329G).

| Binder | TAPIR ID | Format |
|---|---|---|
| Anti-P329G (VH3VL1) × | P1AF4446-004 | Analyte or chemically |
| CD3 (CH2527) 1 + 1 TCB | | immobilized |
| Anti-P329G (VH3VL1) × | P1AF4447-004 | Analyte or chemically |
| CD3 (P035.093) 1 + 1 TCB | | immobilized |
| Anti-P329G (VH3VL1) × | P1AF4448-004 | Analyte or chemically |
| CD3 (P035.093) 2 + 1 TCB | | immobilized |
| human CD3 epsilon-delta- | P1AA6127-015 | Heterodimer of CD3 |
| Fc biotinylated | | epsilon and delta chain |
| | | fused to Fc (immobilized |
| | | via biotin) |
| TCB (P329G) | P1AE7925-003 | Unrelated TCB with P329G |
| | | mutation on Fc (analyte) |
| human Fc (P329G) | P1AD9000-004 | Fc part of human IgG with |
| | | P329G mutation (analyte) |

Human Fc (P329G) was prepared by plasmin digestion of a human IgG1 followed by affinity purification by ProteinA and size exclusion chromatography.

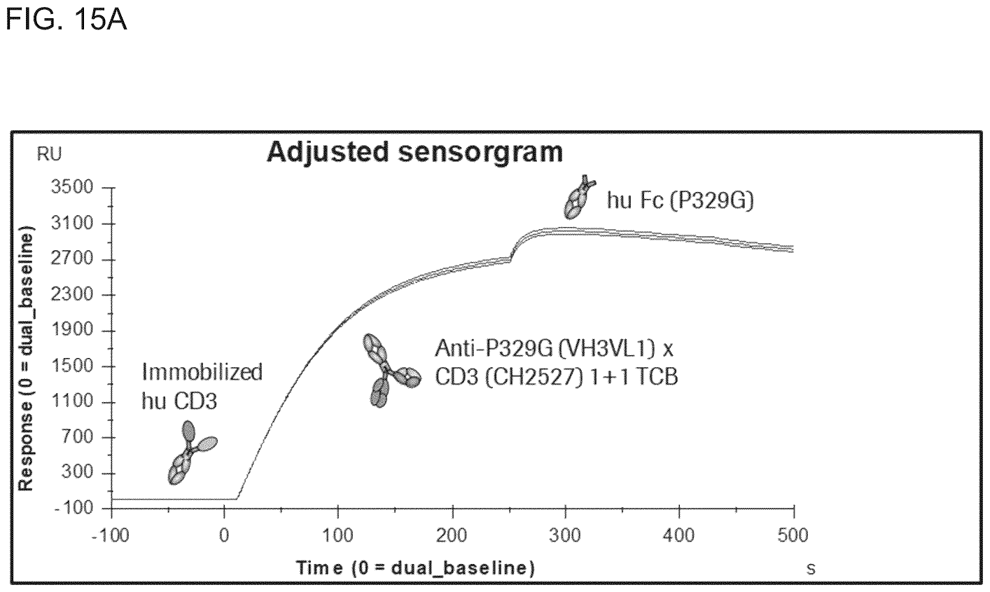

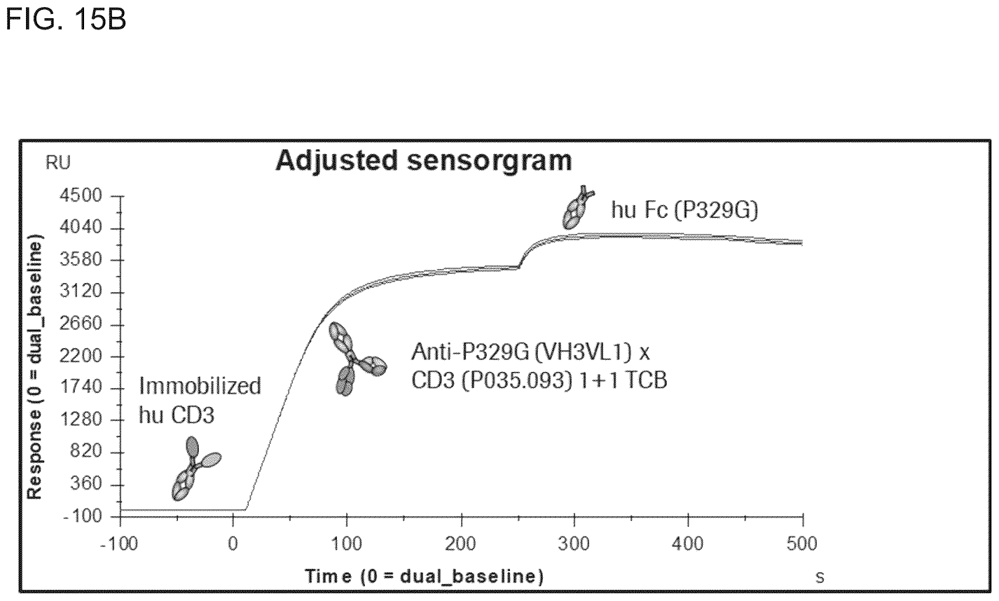

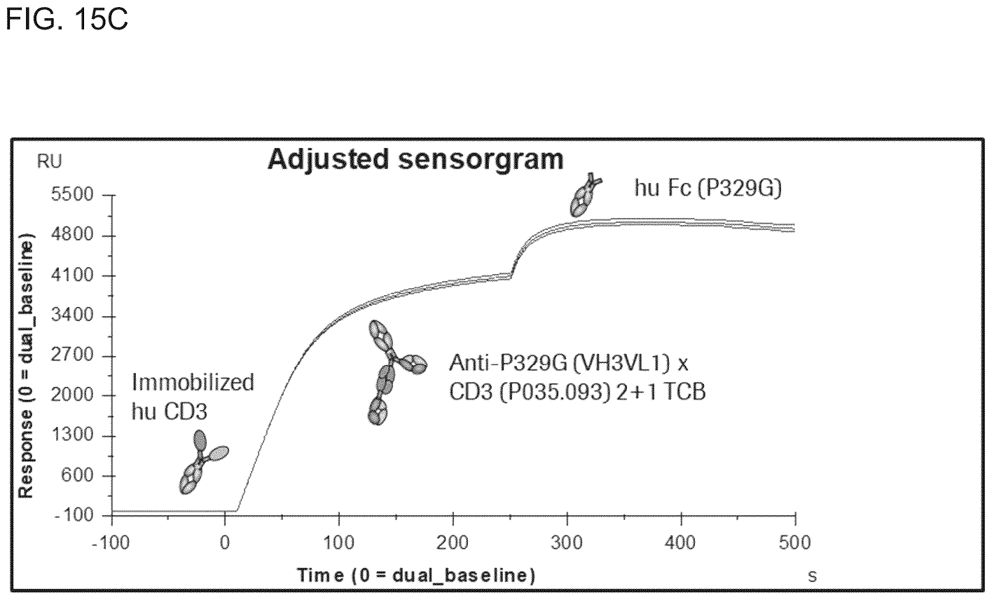

The sensorgrams of simultaneous binding for each TCBs are shown in FIG. 15A-FIG. 15C.

The anti-P329G×CD3 TCBs bind to human CD3 epsilon-delta-Fc as expected. The affinity of the P035.093 binder is around 10-fold higher than for the CH2527 binder (0.3 and 3 nM respectively). The affinity to a construct bearing the P329G mutation varies between 6 and 30 nM and might be influenced by the format of the molecule. In addition, both antigens can be bound at the same time, as expected from a bispecific molecule.

Jurkat NFAT Activation Assay on FolR1+HeLa Cells

Jurkat NFAT activation was measured over a period of 10 h with 2 h intervals. The capacity of tumor targeting huIgG1 and anti-P329G (M-1.7.24)×CD3 (CH2527) 2+1 TCB to induce T cell cross-linking and subsequently T cell activation, was assessed using co-cultures of tumor antigen positive target cells (HeLa) and Jurkat-NFAT reporter cells (a CD3-expressing human acute lymphatic leukemia reporter cell line with a NFAT promoter, GloResponse Jurkat NFAT-RE-luc2P, Promega #CS176501). Upon simultaneous binding of the anti-P329G (M-1.7.24)×CD3 (CH2527) 2+1 TCB to the P329G mutation of the immobilized huIgG1 (bound to the tumor target) and the CD3 antigen (expressed on Jurkat-NFAT reporter cells), the NFAT promoter is activated and leads to expression of active firefly luciferase. The intensity of luminescence signal (obtained upon addition of luciferase substrate) is proportional to the intensity of CD3 activation and signaling.

As tumor targeting antibody a 10 fold decreasing serial titration of anti-FolR1 (16D5) P329G LALA huIgG1 was used in combination with 10-fold decreasing concentrations of the anti-P329G (M-1.7.24)×CD3 (CH2527) 2+1 TCB ranging from the highest final concentration 66 nM to 0.0066 nM of the uTCB. The tumor specific huIgG1 was titrated as well to obtain the highest final concentration of 66 nM to 0.0066 nM. This way the each concentration of the P329G huIgG1 was combined with every concentration of the anti-P329G (M-1.7.24)×CD3 (CH2527) 2+1 TCB, to obtain an optimal activation of the Jurkat-NFAT-reporter cell.

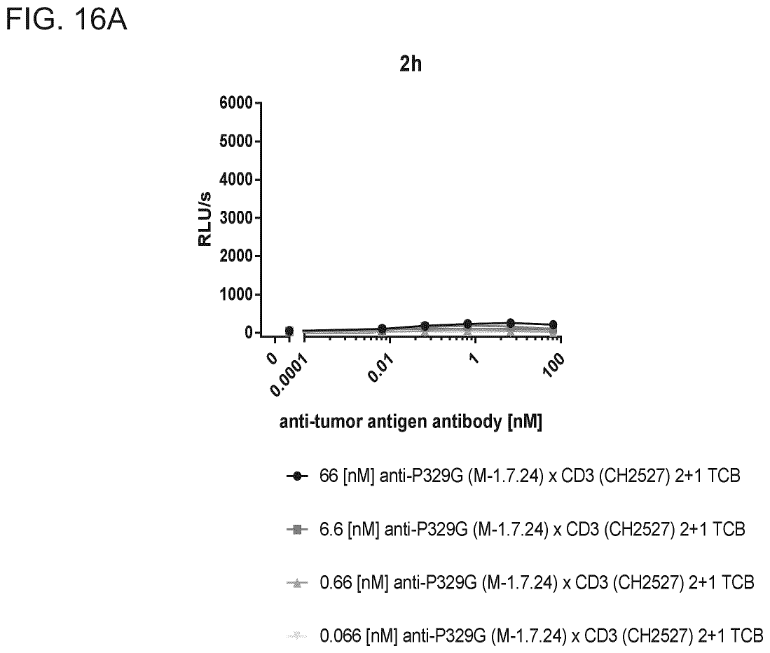

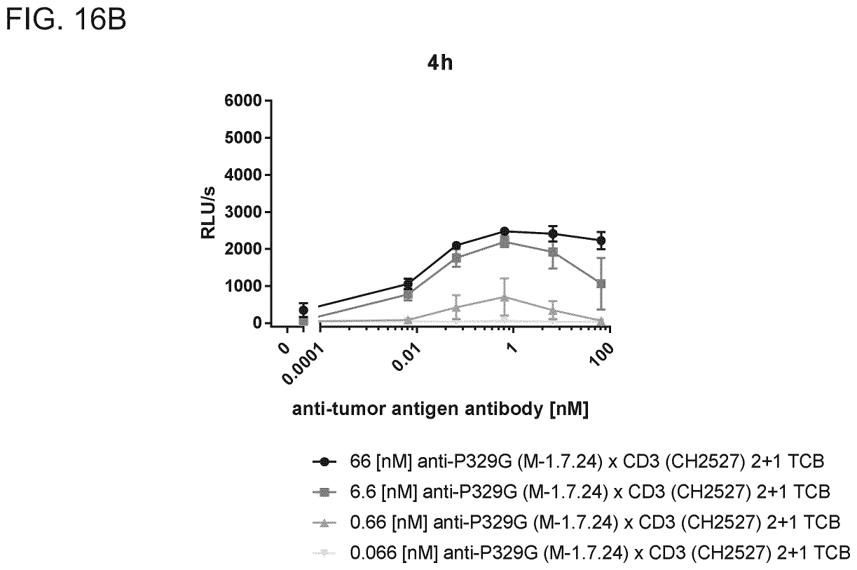

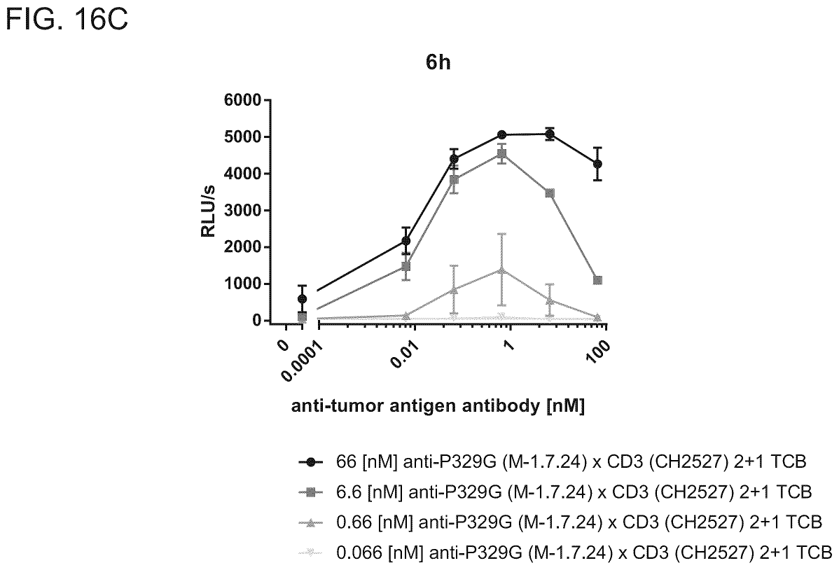

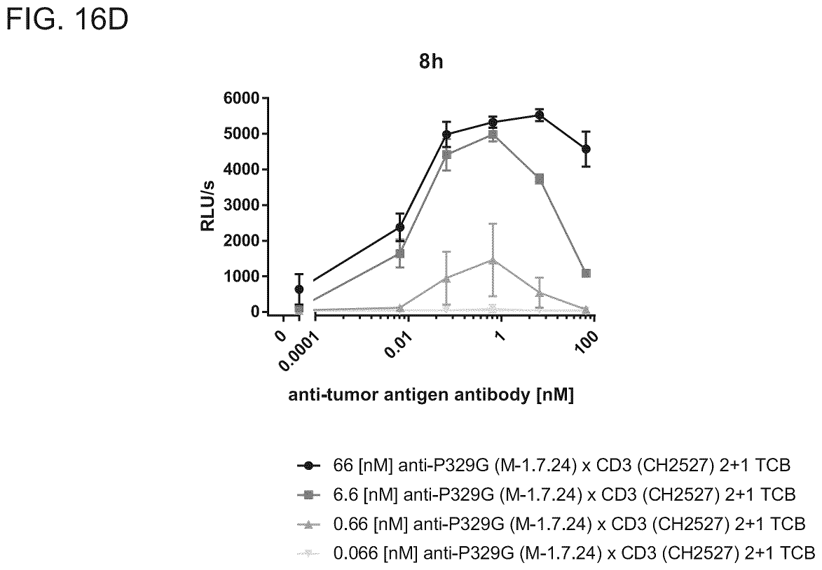

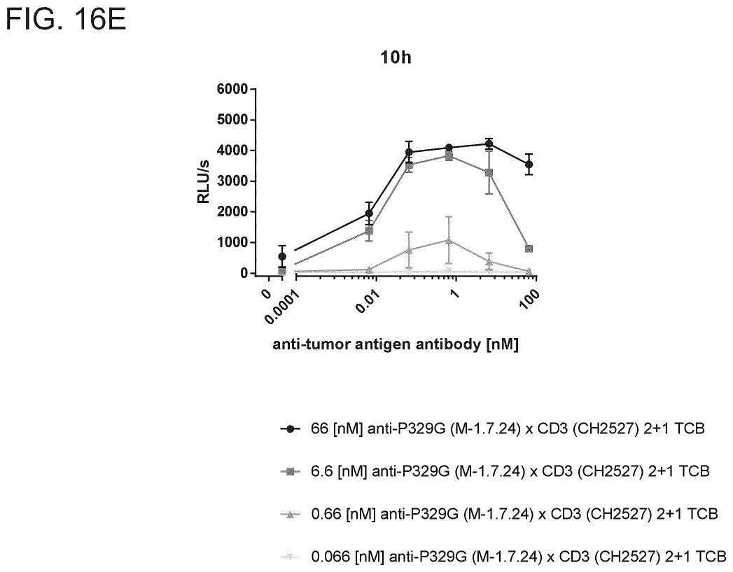

For the assay, HeLa human tumor cells were harvested. Therefore growth medium was removed and cells were washed once with phosphate-buffered saline (PBS, Gibco life technologies). After removing PBS cells were trypsinsed (Trypsin-EDTA (0.05%), phenol red, Gibco). Cell count and viability was determined using ViCell. About 0.002×10⁶ cells/well (10 μl/well) were plated in a flat-bottom, white-walled clear bottom 384-well-plate (Corning #3826) in assay medium (RPMI 1640, 10% FBS and 1% Glutamax), the day before the assay. On the assay day, Jurkat-NFAT reporter cells were harvested. Therefor cells were counted and viability was assessed using ViCell. The needed amount was harvested by centrifugation 5 min at 350 g. About 0.01×106 cells/well (10 μl/well) were plated in assay medium to obtain a final E:T of 5:1 Target and effector cells. Subsequently as well as the different antibody's were seeded simultaneously into a 384 well plate into a final volume of 40 μl. As substrate GloSensor™ cAMP Assay (E1290, Promega) was used according to the manufactures protocol allowing for a kinetic measurement of relative luminescence units (RLU). Readout was performed every 2 h using a TecanReader with temperature control and humidified atmosphere, allowing the automatized measurement without disturbing the culture conditions (37° C. and 5% CO₂). Each point in FIG. 16 represents the mean value of technical triplicates of one experiment. Standard deviation is indicated by error bars. As optimal conditions for the Jurkat-NFTA reporter assay using anti-P329G (M-1.7.24)×CD3 (CH2527) 2+1 TCB in combination with P329G huIgG1 a concentration of 66 nM uTCB and 6.6 nM P329G huIgG1 was determined, whereby the readout should be performed after 6-8 h.

Jurkat NFAT Activation Assay on CD20⁺ z-138 Cells

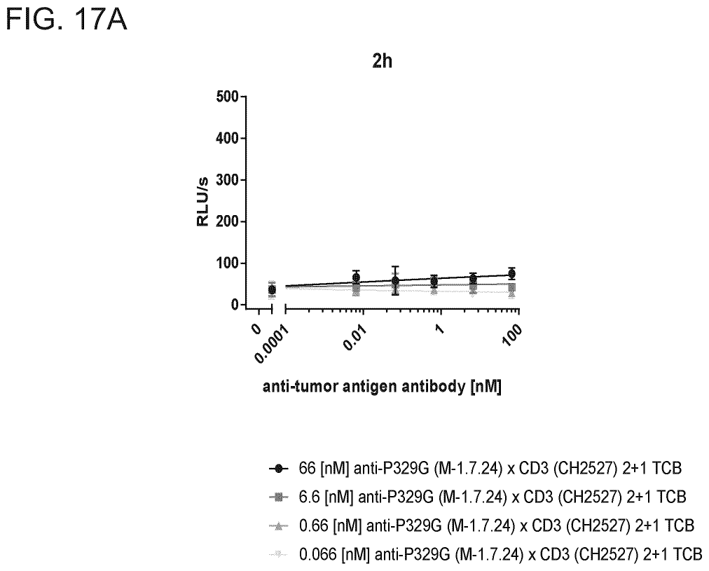

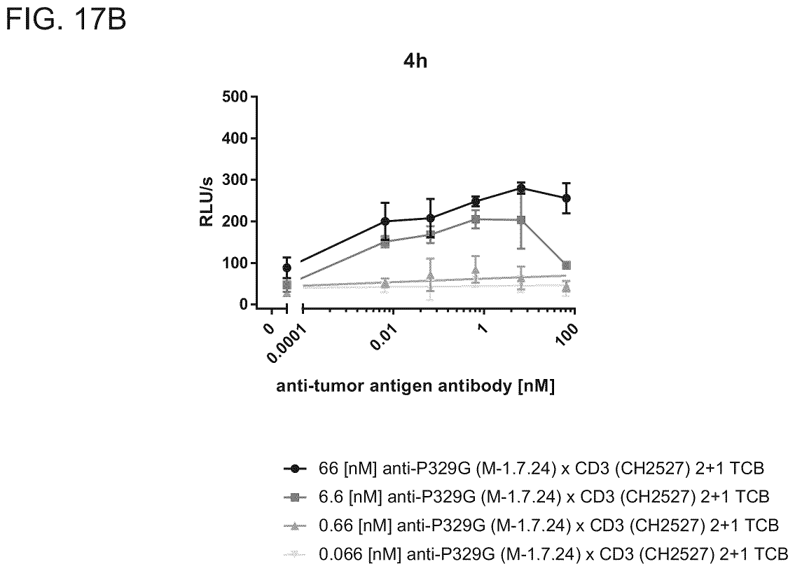

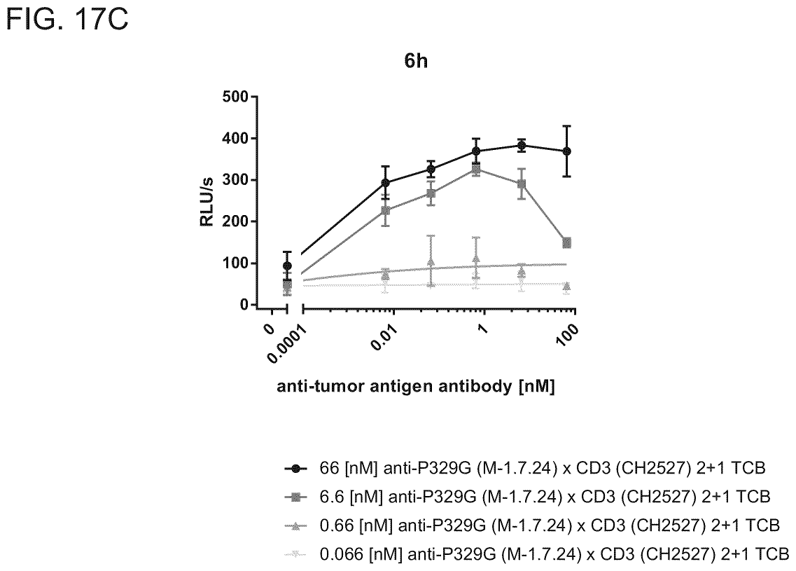

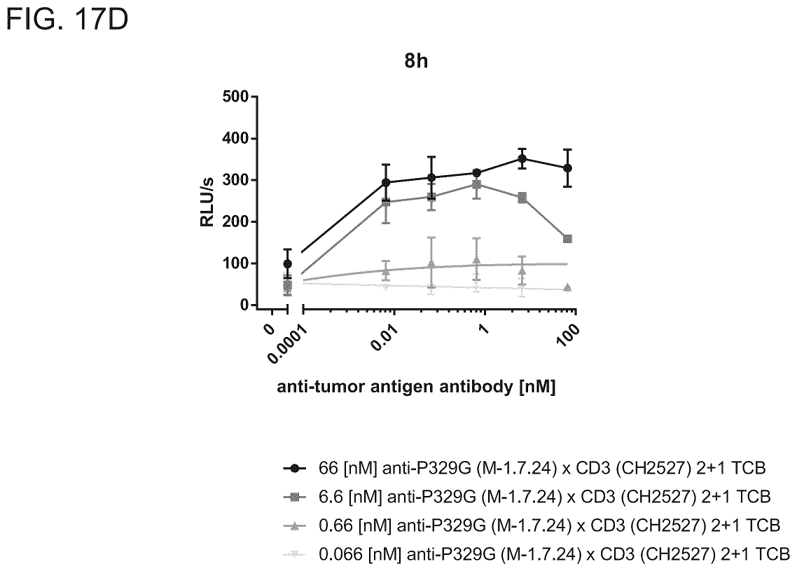

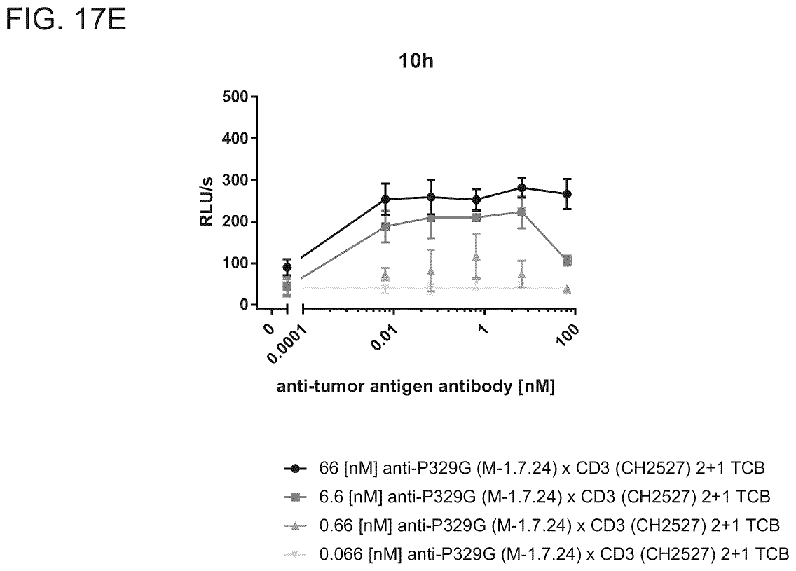

Jurkat NFAT activation was measured over a period of 10 h with 2 h intervals. The assay was performed as described above. Instead of HeLa cells, z-138 target cells were used. As tumor targeting antibody a titration of anti-CD20 P329G LALA huIgG1 was used in combination with different concentrations of the anti-P329G (M-1.7.24)×CD3 (CH2527) 2+1 TCB ranging from a final concentration of 66 nM to 0.0066 nM of uTCB. The tumor specific huIgG1 was titrated as well to obtain a final concentration of 66 nM to 0.0066 nM. Each point in FIG. 17 represents the mean value of technical triplicates of one experiment. Standard deviation is indicated by error bars. As optimal conditions for the Jurkat-NFTA reporter assay using uTCB in combination with P329G huIgG1 a concentration of 66 nM uTCB and 6.6 nM P329G huIgG1 was determined, whereby the readout should be performed after 6-8 h.

Jurkat NFAT Activation Assay on FAP⁺ MV3 Cells

Jurkat NFAT activation was measured over a period of 10 h with 2 h intervals. The assay was performed as described above. Instead of HeLa cells, MV3 (FAP⁺) target cells were used. As tumor targeting antibody a titration of anti-FAP P329G LALA huIgG1 was used in combination with different concentrations of the anti-P329G (M-1.7.24)×CD3 (CH2527) 2+1 TCB ranging from a final concentration of 66 nM to 0.0066 nM of TCB. The tumor specific IgG was titrated as well to obtain a final concentration of 66 nM to 0.0066 nM. This way the each concentration of the P329G IgG was combined with every concentration of the uTCB, to obtain an optimal activation of the Jurkat-NFAT-reporter cell.

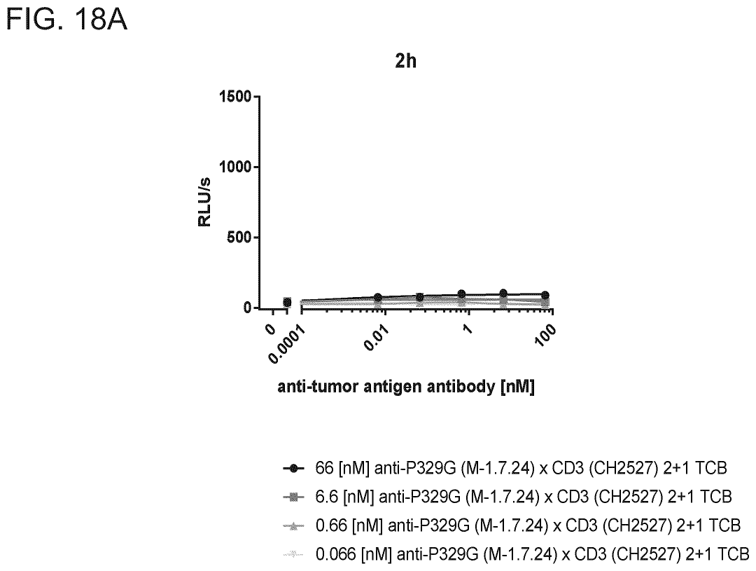

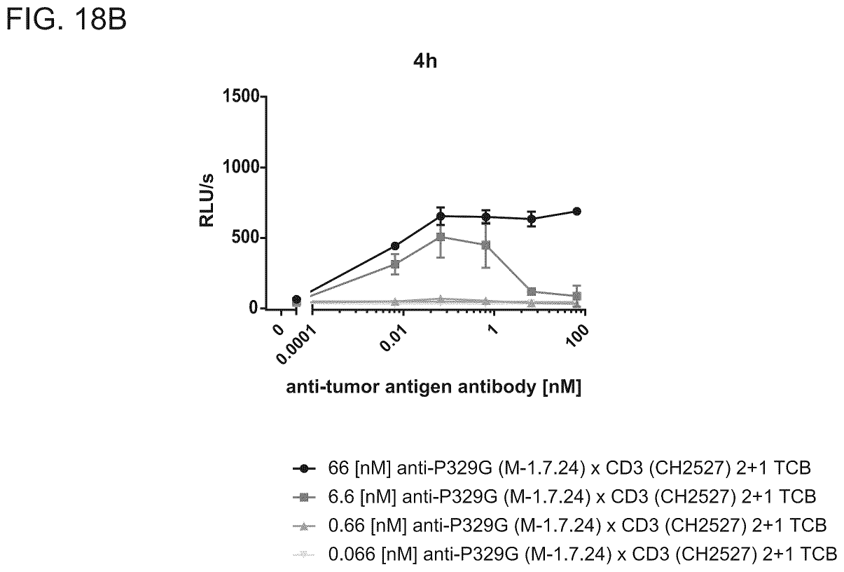

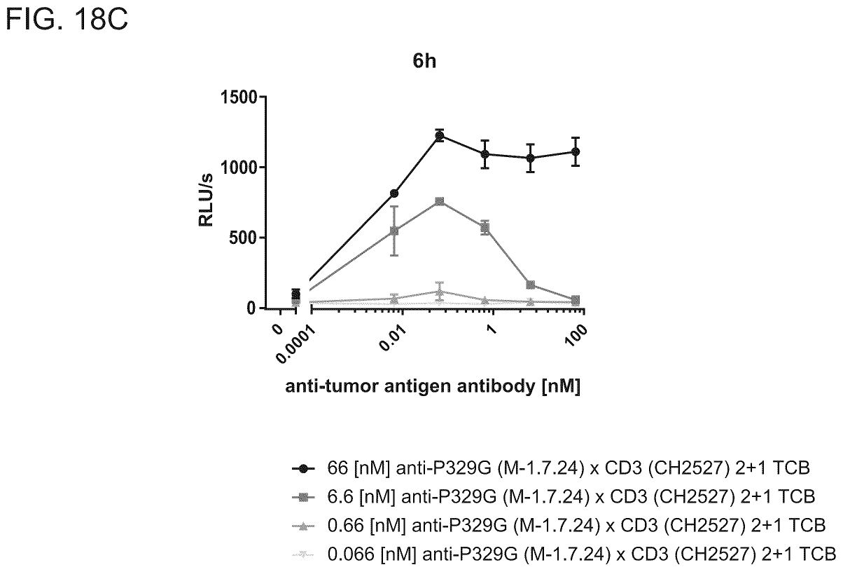

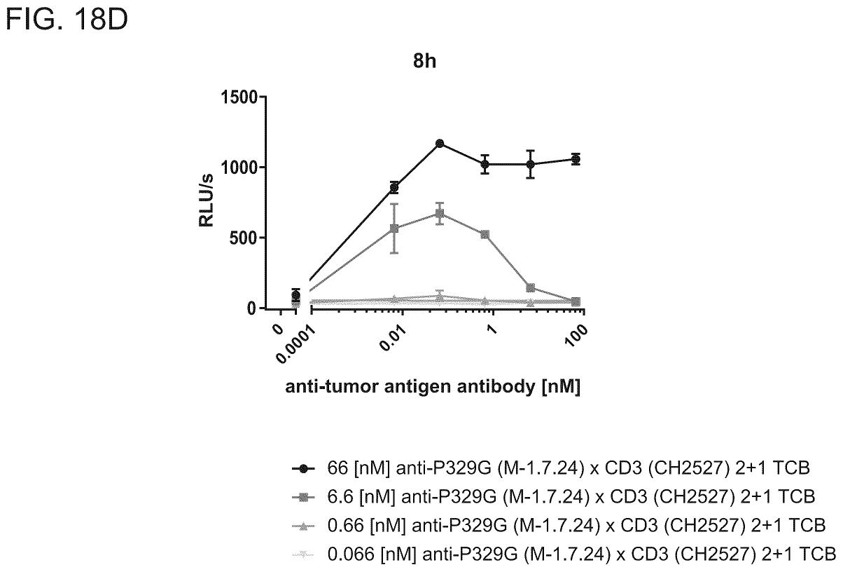

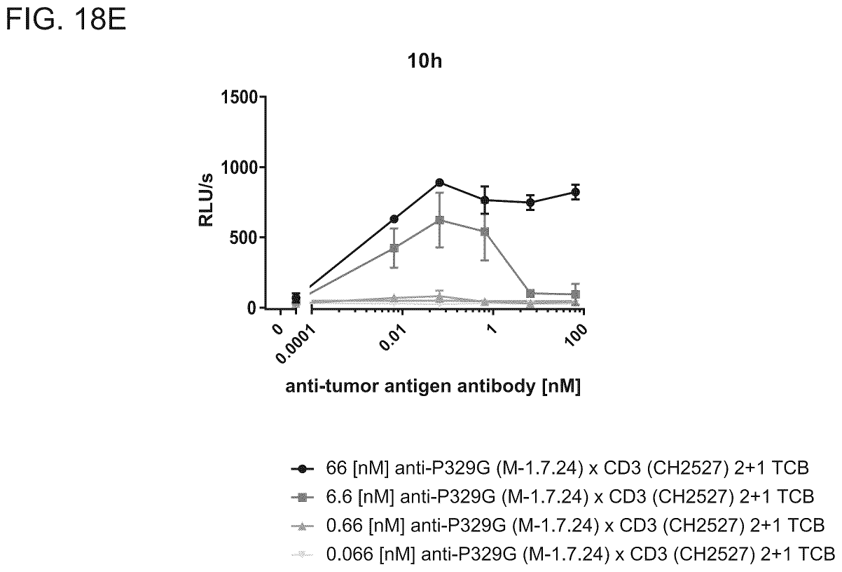

The capacity of tumor targeting huIgG1 and anti-P329G (M-1.7.24)×CD3 (CH2527) 2+1 TCB to induce T cell cross-linking and subsequently T cell activation, was assessed using co-cultures of tumor antigen positive target cells (MV3) and Jurkat-NFAT reporter cells (a CD3-expressing human acute lymphatic leukemia reporter cell line with a NFAT promoter, GloResponse Jurkat NFAT-RE-luc2P, Promega #CS176501). Upon simultaneous binding of the TCB to the P329G mutation of the immobilized huIgG1 (bound to the tumor target) and the the CD3 antigen (expressed on Jurkat-NFAT reporter cells), the NFAT promoter is activated and leads to expression of active firefly luciferase. The intensity of luminescence signal (obtained upon addition of luciferase substrate) is proportional to the intensity of CD3 activation and signaling. Each point in FIG. 18 represents the mean value of technical triplicates of one experiment. Standard deviation is indicated by error bars. As optimal conditions for the Jurkat-NFTA reporter assay using anti-P329G (M-1.7.24)×CD3 (CH2527) 2+1 TCB in combination with P329G huIgG1 a concentration of 66 nM TCB and 6.6 nM P329G huIgG1 was determined, whereby the readout should be performed after 6-8 h.

Jurkat NFAT Activation Assay on CD20⁺Z-138 Cells and SU-DHL-4 Cells

Jurkat NFAT activation was measured in the presence of CD20 targeting P329G LALA huIgG1 with the anti-P329G (M-1.7.24)×CD3 (CH2527) 2+1 TCB targeting the P329G mutation in the Fc of the targeting antibody and the CD3 on T cells. As tumor targeting antibody a titration of anti-CD20 P329G LALA IgG1 was used in combination with different concentrations of anti-P329G (M-1.7.24)×CD3 (CH2527) 2+1 TCB both ranging from the highest final concentration of 66 nM to 0.0066 nM.

The Jurkat NFAT reporter cell line is a CD3-expressing human acute lymphatic leukemia reporter cell line with a NFAT promoter (GloResponse Jurkat NFAT-RE-luc2P, Promega #CS176501). Upon simultaneous binding of the TCB to the P329G mutation of the immobilized huIgG1 (bound to the tumor target) and the CD3 antigen (expressed on Jurkat-NFAT reporter cells), the NFAT promoter is activated and leads to expression of active firefly luciferase. The intensity of luminescence signal (obtained upon addition of luciferase substrate) is proportional to the intensity of CD3 activation and signaling.

For the assay, tumor target cells z-138 (CD20⁺) or SU-DHL-4 (20⁺), counted and checked for their viability using ViCell. The desired amount tumor target cells was harvested by centrifugation 5 min at 350 g. Cells were resuspended in assay medium (RPMI1640+10% FBS und 1% Glutamax) and 0.002×10⁶ cells/well (10 μl/well) each were plated in a flat-bottom, white-walled clear bottom 384-well-plate (Corning #3826). Subsequently, Jurkat-NFAT reporter cells were harvested and viability assessed using ViCell. Cells were plated at 0.01×10⁶ cells/well (10 μl/well) to obtain a final E:T of 5:1. Serial dilution of the uTCB and the IgG were prepared in assay medium. 10 μl/well each, of the IgG and 10 μl/well of the desired uTCB concentration were added to the respective wells in the 384 well plate. The final assay volume was 40 μl.

After an incubation time of 7 h, 20% of ONE-Glo™ Luciferase Assay readout (Promega, E6120) was added and the readout was performed immediately using Tecan® plate reader, measuring relative luminescence units (RLU). Each point represents the mean value of technical triplicates of one experiment. Standard deviation is indicated by error bars.

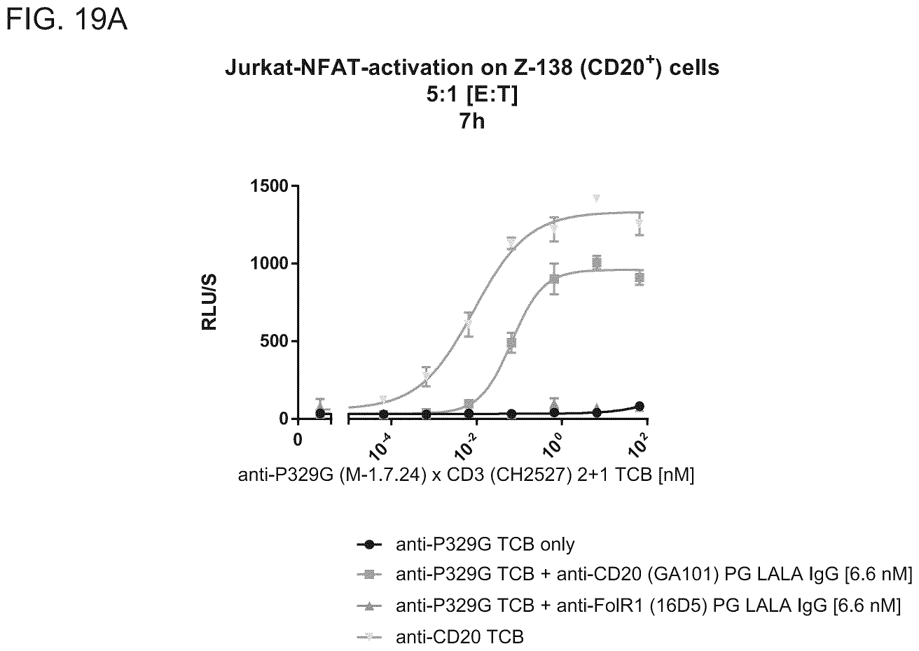

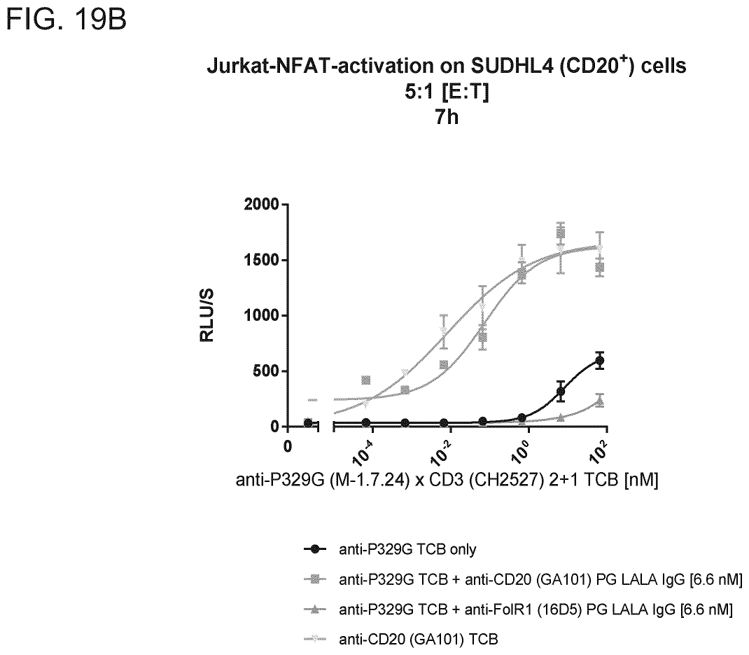

The anti-P329G (M-1.7.24)×CD3 (CH2527) 2+1 TCB alone does not show any activation of the target cells also the control condition where anti-FolR1 P329G huIgG1 antibody as primary antibody was used (FolR1 is not expressed on the target cells) does not show any Jurkat activation. Activation of Jurkat NFAT cells could be observed when the primary anti-CD20 P329G LALA huIgG1 antibody in combination with the TCB was used (FIG. 19). This activation is dose-dependent.

Jurkat NFAT Activation Assay on CD20⁺ SU-DHL-4 Cells

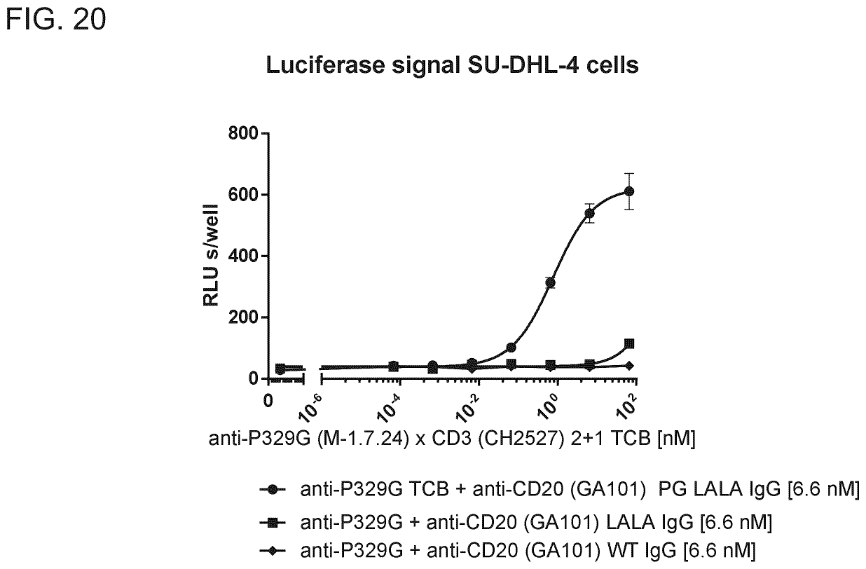

The capacity of the anti-P329G (M-1.7.24)×CD3 (CH2527) 2+1 TCB to induce specific T cell cross-linking and subsequently T cell activation was assessed using co-cultures of tumor target cells and Jurkat-NFAT reporter cells (a CD3-expressing human acute lymphatic leukemia reporter cell line with a NFAT promoter, GloResponse Jurkat NFAT-RE-luc2P, Promega #CS176501). The assay was performed as described in above. Binding specificity of the anti-P329G (M-1.7.24)×CD3 (CH2527) 2+1 TCB to the P329G mutation was assessed using a titration of anti-CD20 P329G LALA huIgG1, anti-CD20 LALA huIgG1 or anti-CD20 wildtyp-Fc huIgG1 in a co-culture of SU-DHL-4 (CD20⁺) tumor cells and Jurkat-NFAT reporter cells. All antibodies were titrated starting from 6.6 nM (1:10 dilution series) in combination with the anti-P329G (M-1.7.24)×CD3 (CH2527) 2+1 TCB. This assay shows the specificity of the TCB since and activation of the reporter cell line is detectable only if the tumor antigen targeting CD20 antibody with the P329G mutation is used in combination with the TCB (FIG. 20).

Killing of Adherent Tumor Cells

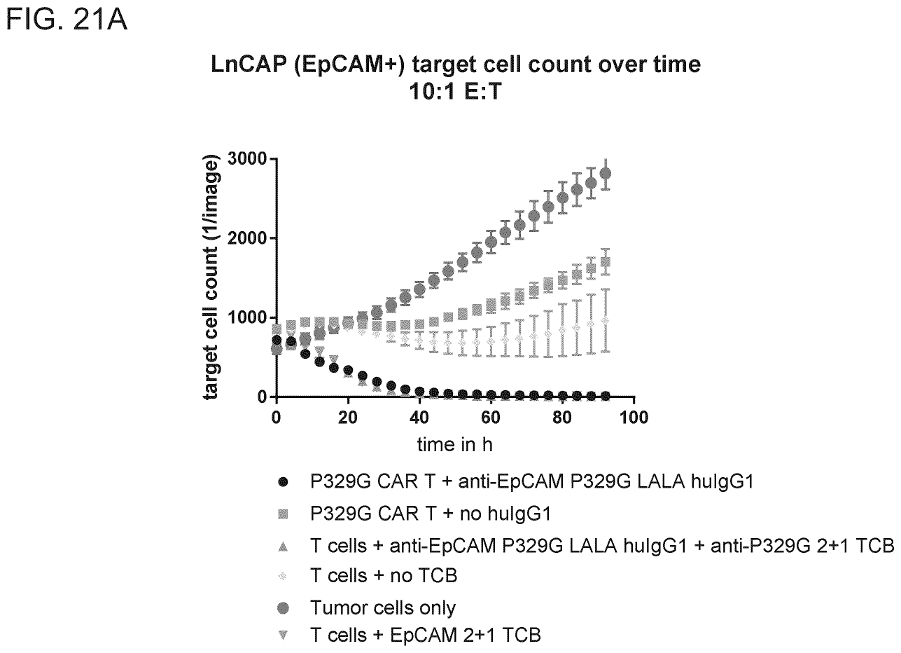

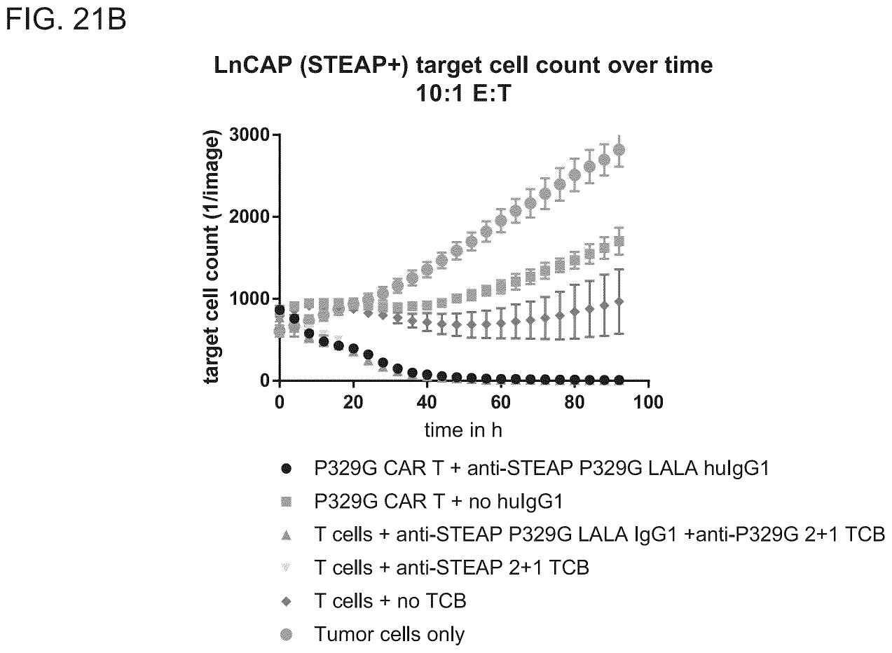

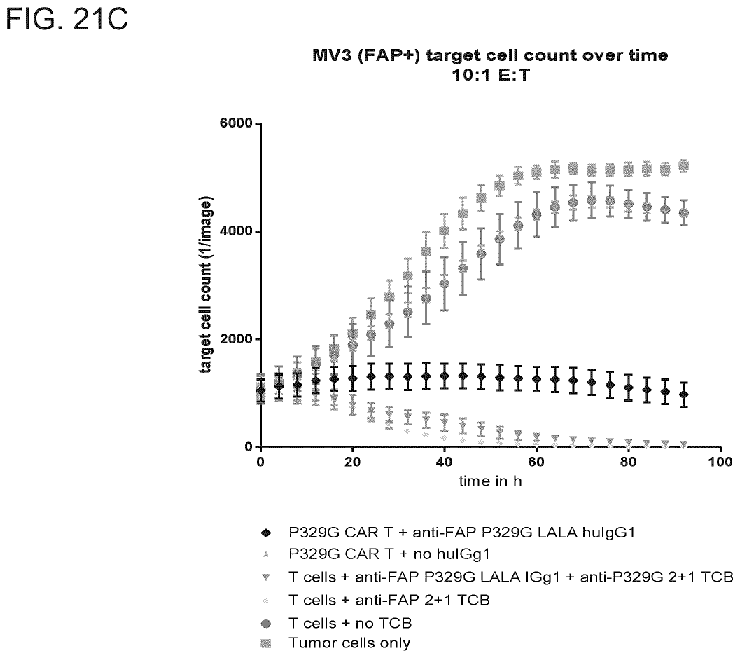

Killing of adherent tumor cells was assessed by quantification of red nuclear cell counts over time using the live cell imaging device Incucyte. Adherent target cells were harvested, counted and checked for their viability using vicell counter. The day before the experiment cells were adjusted to the desired cell density, in assay medium (advanced RPMI 1640+10% FBS+1% Glutamax+Pen/Strep) and seeded in 100 μl assay medium to ensure proper adhering to the wells. As assay plate a flat bottom transparent 96 well plate from TPP was used. As effector cells human PBMCs or anti-P329G CAR T cells (specific against the same mutation as the anti-P329G TCB) were counted and checked for their viability using Vicell counter. Cells were harvested by centrifugation (5 min and 350 g) and adjusted to the desired cell density. Cells were seeded in assay medium at a E:T ratio of 10:1. Cells are seeded in 50 or assay-medium. Preparation of the antibodies: Antibodies were diluted in assay medium and 50 ul of IgG and/or anti-P329G (M-1.7.24)×CD3 (CH2527) 2+1 TCB were added to the respective wells (anti-EpCAM: FIG. 21A, anti-STEAP: FIG. 21B or anti-FAP: FIG. 21C) to obtain a final concentration of 66 nM TCB and/or 6.6 nM tumor targeting P329G huIgG1. The plate was placed in the IncuCyte (37° C. and 5% CO₂ humidified atmosphere). The target cell count per image was assessed using Essen BioScience software. Depicted is a representative graph showing tumor cell reduction over time assessed by red nuclear count per image (FIG. 21A-FIG. 21C). It can be observed that the tumor cell growth is impaired when either the anti-P329G (M-1.7.24)×CD3 (CH2527) 2+1 TCB in combination with the respective tumor specific P329G possessing huIgG1 is used. Further tumor cell growth is also impaired when the respective TCB directly targeting the respective antigen is used or anti-P329G CAR T cells in combination with the tumor specific P329G possessing huIgG1.

Activation of T Cells by Different T Cell Activating Fc Binding Molecule Formats

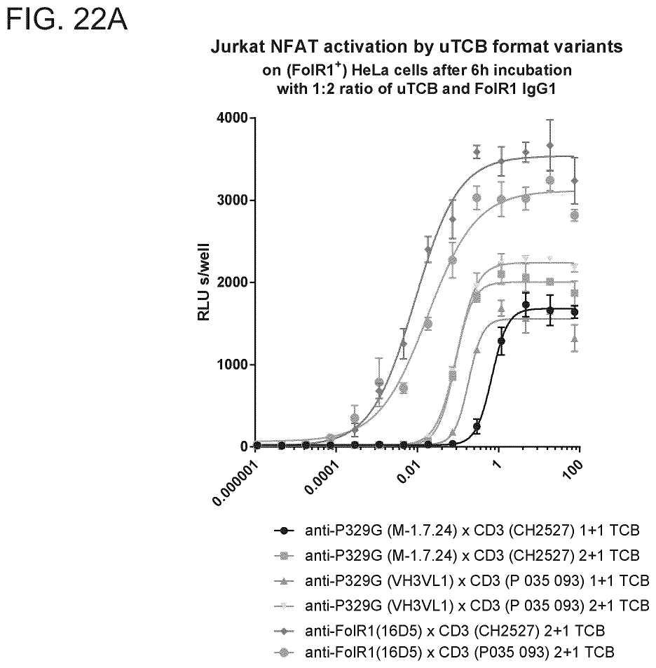

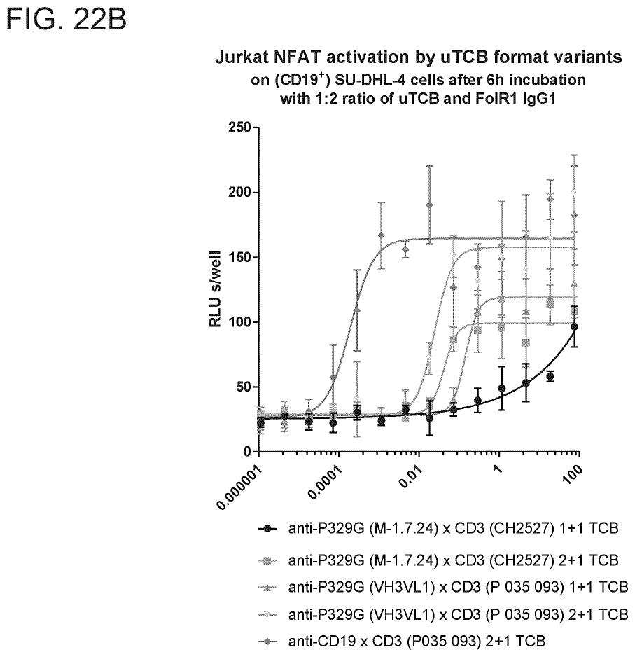

The capacity of 2+1 or 1+1 anti-P329G TCB to induce specific T cell cross-linking and subsequently T cell activation was assessed using co-cultures of tumor target cells and Jurkat-NFAT reporter cells (a CD3-expressing human acute lymphatic leukemia reporter cell line with a NFAT promoter, GloResponse Jurkat NFAT-RE-luc2P, Promega #CS176501) As described above. Upon simultaneous binding of the anti-P329G TCB to the P329G possessing tumor opsonizing huIgG1 and binding to the CD3 antigen (expressed on Jurkat-NFAT reporter cells), the Jurkat NFAT promoter is activated and leads to expression of active firefly luciferase. The intensity of luminescence signal is proportional to the intensity of CD3 activation and signaling and can be measured upon addition of a luciferase substrate. For the assay, tumor target SUDHL-4 cells were and antibody dilution rows of the TCB titrated starting with the highest concentration of 75 nM (in 1:4 dilution series) or huIgG1 starting with the highest concentration of 150 nM (also with a dilution series of 1:4). 10 μl/well of each antibody was added to the respective wells. Experiment was performed with a final TCB to huIgG1 ratio of 1:2. The final assay volume was 40 μl per well. This assay was performed to compare the anti-P329G×CD3 2+1 TCB with the anti-P329G×CD3 1+1 TCB format with M-1.7.24 or humanized VH3VL1 P329G binder in combination with CD3 binder variants CH2527 or P035 039 (FIG. 22). It is demonstrated that independent of the TCB format, the murin anti-P329G (M-1.24) binder in combination with the (CH2527) CD3 binder are less potent that the TCB with humansied anti-P329G binder and the P035 039 CD3 binder. This experiment was performed like the one above, except SU-DHL-4 target cells were used and CD20 targeting huIgG1. FIG. 22 shows that anti-P329G (VH3VL1) x CD3 (P 035 093) 2+1 TCB performs best in terms of EC50 and plateau value.

HeLa Target Cell Lysis by Human PBMCs

Tumor cell lysis was determined by the measurement of released LDH after 5.5 h, 24 h and 48 h upon incubation with the anti-P329G (VH3VL1)×CD3 (P 035 093) 2+1 universal TCB or anti-P329G (VH3VL1)×CD3 (P 035 093) 1+1 universal TCB (uTCB). Target cells were harvested, counted and checked for their viability. 0.03×10⁶ cells/well were plated in 100 μl of their culture medium in a flat-bottom 96-well plate from TPP.

The next day PBMC effector cells were isolated from buffy coats, obtained from Zurich blood donation center in accordance with the Declaration of Helsinki. The buffy coat was diluted 2:1 with PBS and human PBMCs were isolated by density gradient centrifugation (450×g, 30 min at room temperature without break) over Histopaque-1077 (Sigma-Aldrich #10771). PBMCs were harvested from the interphase, washed at three times with PBS for 10 min at 350 g. After washing PBMCs were counted the desired amount was seeded in the respective wells of the assay plate in a 5:1 E:T ratio in assay medium in 100 ul volume.

Serial dilution of the uTCB and IgG were prepared in 1:10 dilution steps in assay medium. The uTCB to IgG ratio was 1:2, with final uTCB concentrations from 75 nM to 0.0000075 nM and final IgG concentrations ranging from 150 nM to 0.0000150 nM.

Target cell killing was assessed after 5.5 h 24 h and 48 h of incubation by quantification of LDH released into cell supernatants by apoptotic/necrotic cells (LDH detection kit, Roche Applied Science, #11 644 793 001). Maximal lysis of the target cells (=100%) was achieved by incubation (1 h at 37° C. and 5 Co2 in a humidified incubator) of target cells with a final concentration of 1% Triton X-100 per control well. Minimal lysis (=0%) refers to target cells co-incubated with effector cells without bispecific antibody. Using a multichannel pipette, 50 ul of supernatant was transferred to a transparent 96 well plate and 1:1 freshly prepared Cytotoxicity reagent was prepared according to the manufacturers protocol and absorbance was measured with Tecan Spark reader over an interval of 10 min.

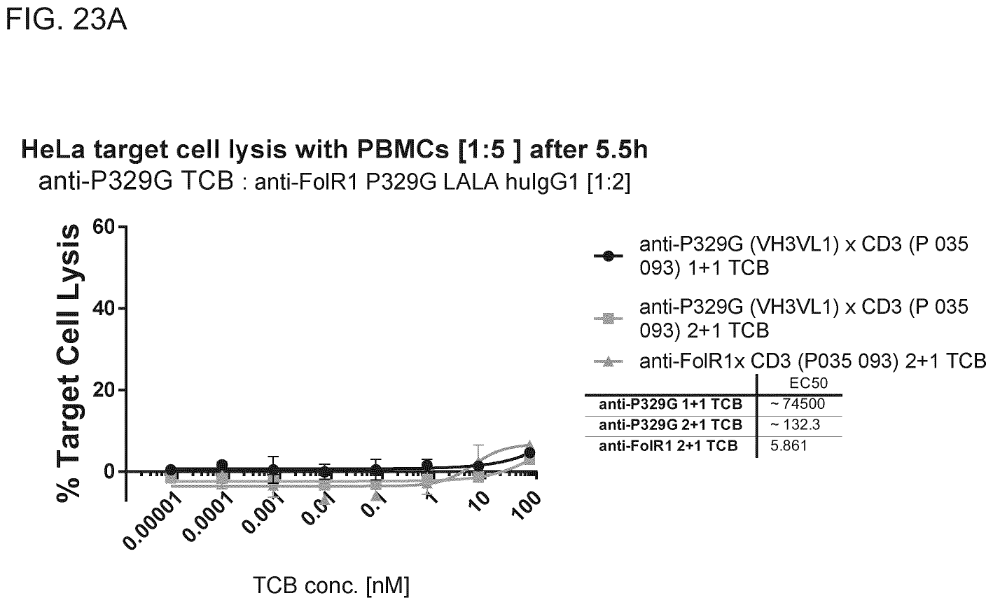

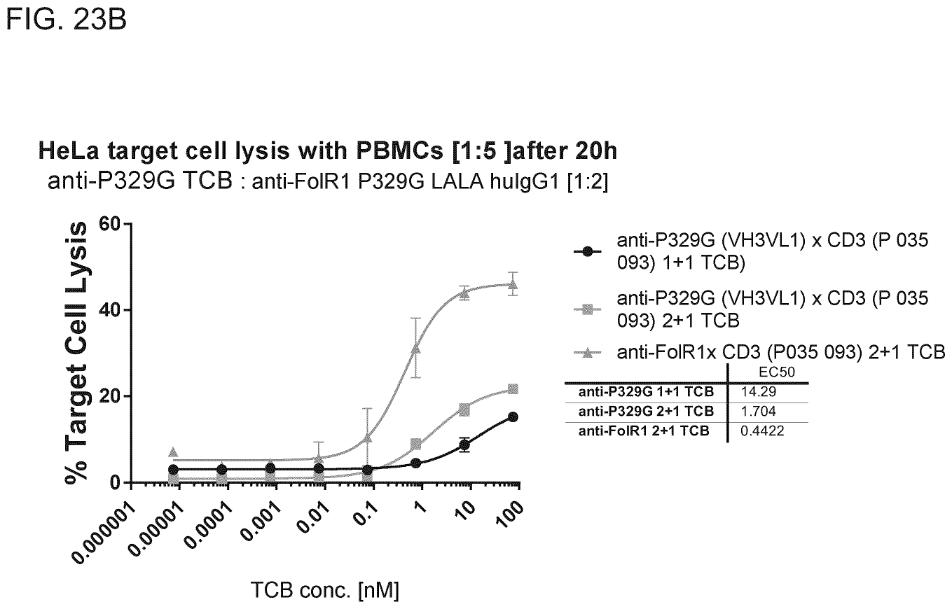

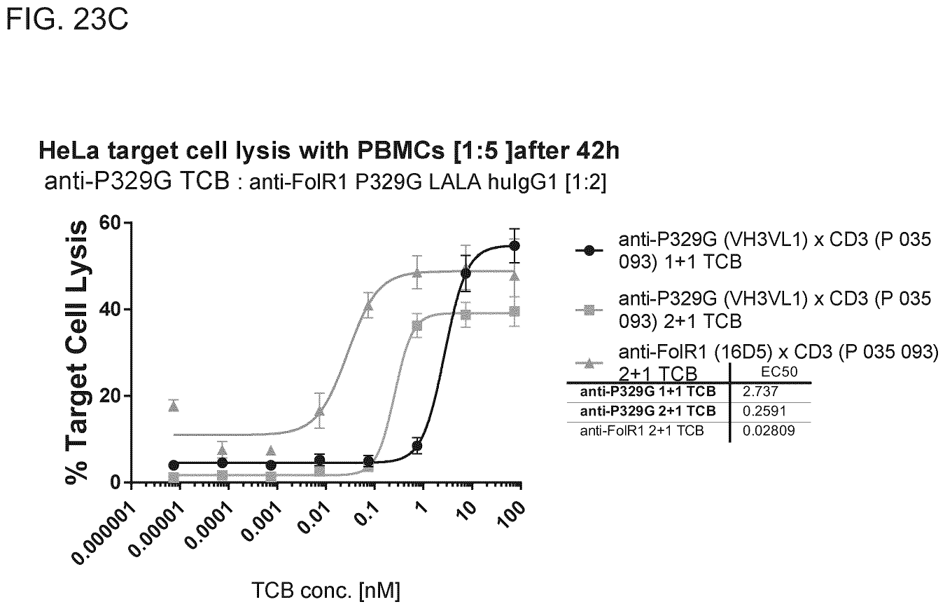

Depicted are dose-fitting curves of technical average values from triplicates, error bars indicate SD, calculated using GraphPadPrism7. FIG. 23 shows after 5.5 h incubation (FIG. 23A) no tumor cell lysis was induced. After 20 h (FIG. 23B) tumor lysis started to be detectable for both formats uTCB in 1+1 format as well as in the 2+1 format. After 42 h (FIG. 23C) the measured tumor cell lysis increased for both formats but the anti-P329G (VH3VL1)×CD3 (P035 093) 2+1 TCB format was superior compared to the anti-P329G (VH3VL1)×CD3 (P 035 093) 1+1 TCB format.

CD19+ Nalm 6 Target Cell Lysis by Human PBMCs

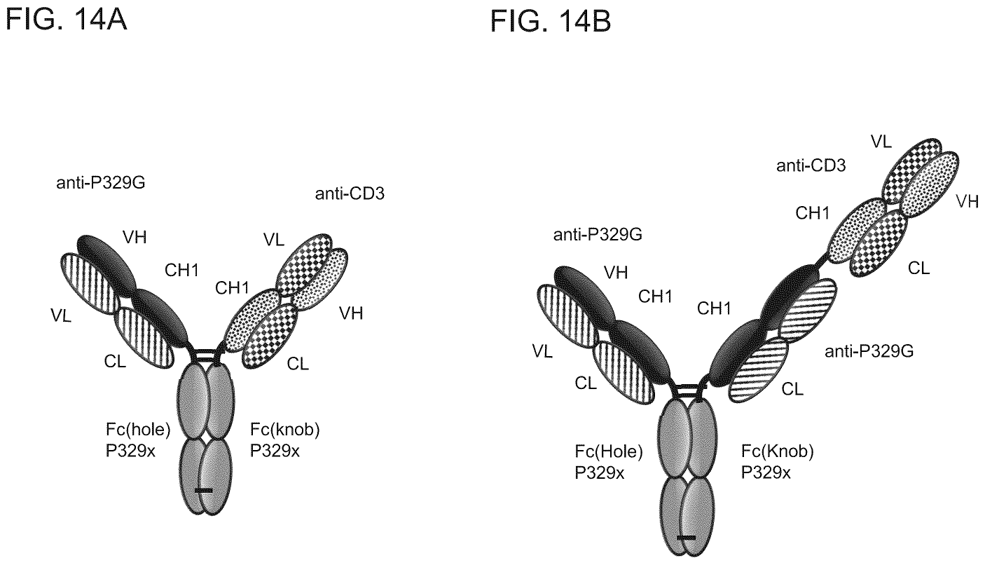

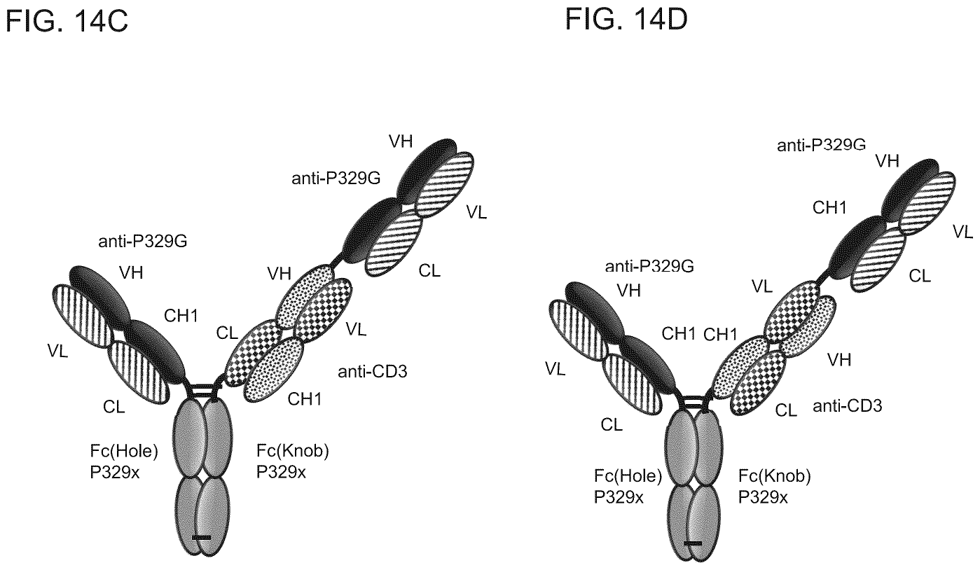

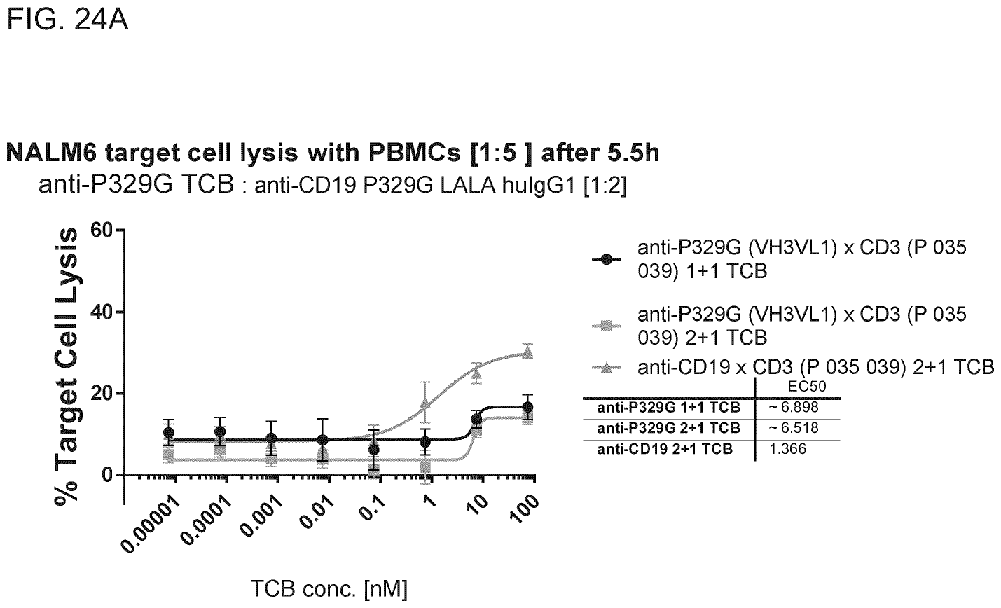

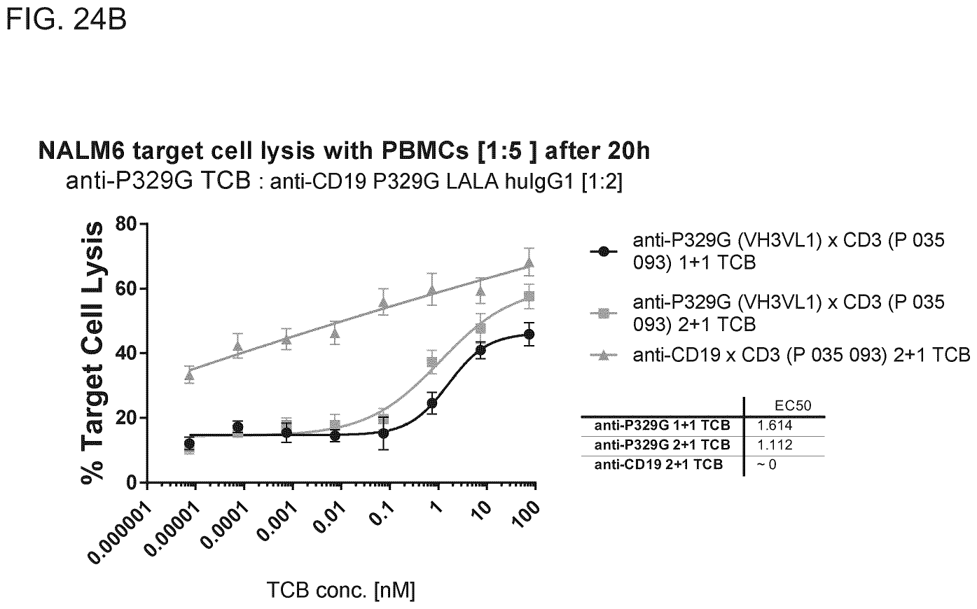

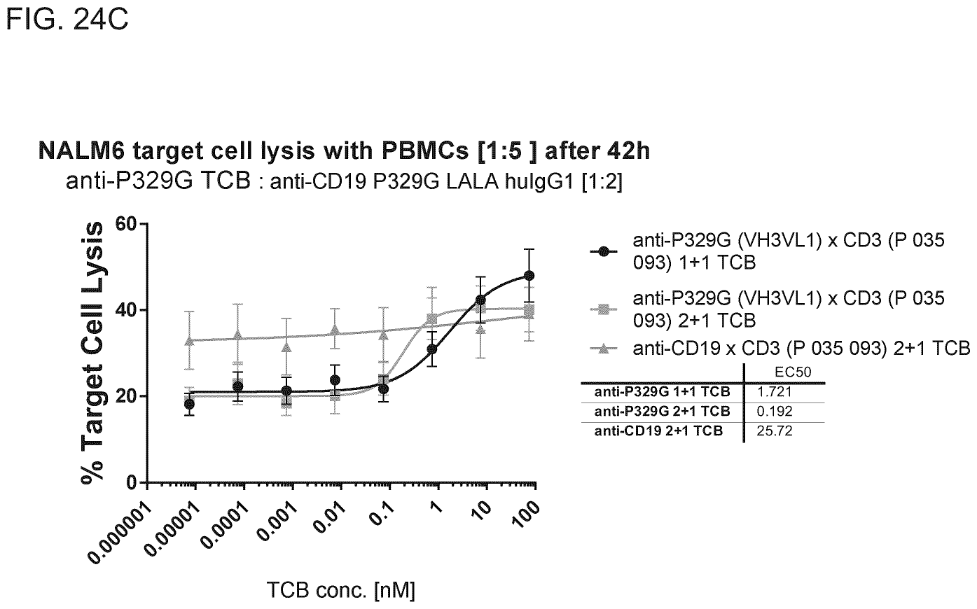

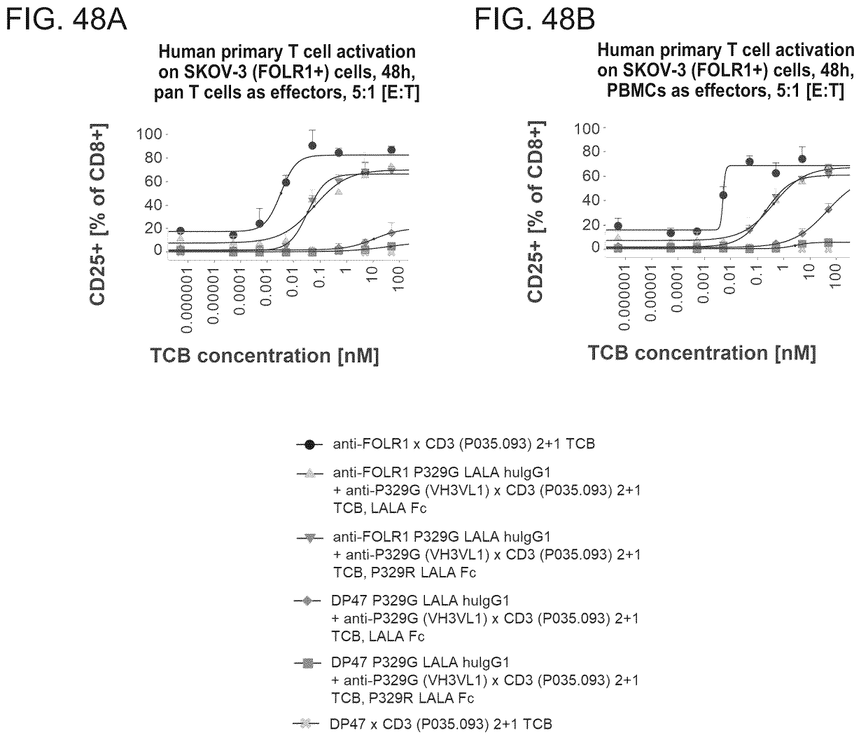

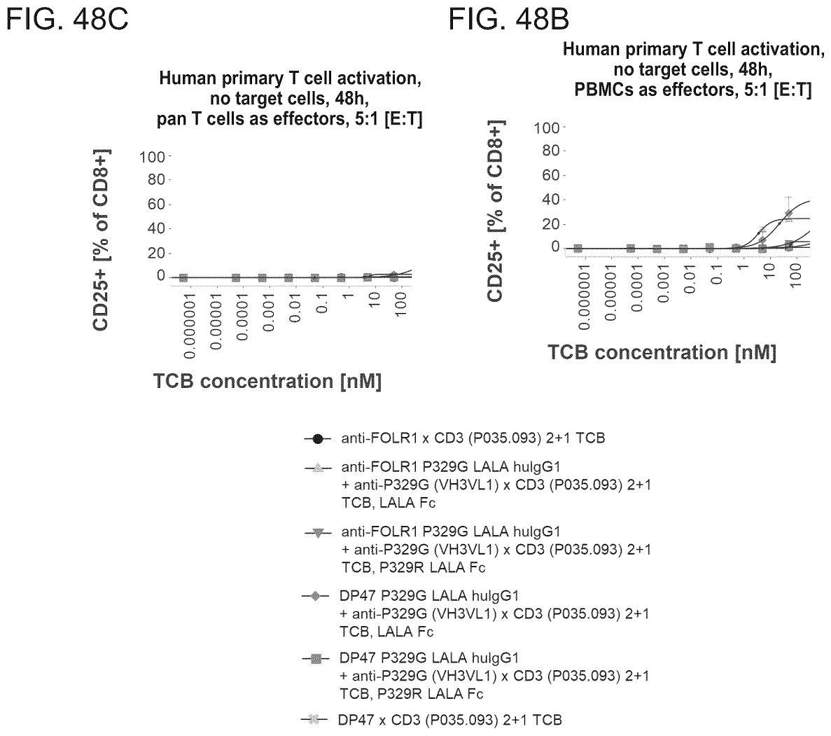

Tumor cell lysis was determined by the measurement of released LDH after 5.5 h (FIG. 14A), 24 h (FIG. 14B) and 48 h (FIG. 14C), upon incubation with the anti-P329G (VH3VL1)×CD3 (P 035 039) 2+1 TCB or anti-P329G (VH3VL1)×CD3 (P 035 039) 1+1 TCB format (uTCBs). Target cells were harvested, counted and checked for their viability. 0.03×10⁶ cells/well were plated in 100 μl of their culture medium in a flat-bottom 96-well plate from TPP. The next day human PBMCs effector cells were isolated from fresh blood by gradient centrifugation over Histopaque (Sigma) and seeded in 50 μl advanced RPMI1640 containing 2% FCS and 1% GlutaMax (assay medium). Serial dilution of the uTCBs and IgG were prepared in 1:10 dilution steps in assay medium. The uTCB to IgG ratio was 1:2, with final uTCB concentrations from 75 nM to 0.0000075 nM and final IgG concentrations ranging from 150 nM to 0.0000150 nM. Tumor cell lysis was assessed after 5.5 h, 20 h and 42 h by calorimetric quantification of lactate dehydrogenase (LDH) release. Maximal lysis of the target cells (=100%) was achieved by incubation of target cells with 1% Triton X-100. Minimum lysis (=0%) refers to target cells co-incubated with effector cells without bispecific antibody. Depicted are dose-fitting curves of technical average values from triplicates, error bars indicate SD, calculated using GraphPadPrism7. FIG. 24A shows no detectable tumor cell lysis after 5.5 h. After 20 h tumor lysis started to be detectable for both formats uTCB in 1+1 format as well as in the 2+1 format FIG. 24B). After 42 h the measured tumor cell lysis increased for both formats but the anti-P329G (VH3VL1)×CD3 (P 035 093) 2+1 TCB format was superior compared to the anti-P329G (VH3VL1)×CD3 (P 035 093) 1+1 TCB format (FIG. 24C).

### Example 4

### Fc Binding Molecules Comprising Costimulatory (CD28) Immune Activating Moiety

Cloning of the Extracellular Domain of CD28

A DNA fragment encoding the extracellular domain (amino acids 1 to 134 of matured protein) of human CD28 (Uniprot: P10747) was inserted in frame into two different mammalian recipient vectors upstream of a fragment encoding a hum IgG1 Fc fragment which serves as solubility- and purification tag. One of the expression vectors contained the “hole” mutations in the Fc region, the other one the “knob” mutations as well as a C-terminal avi tag allowing specific biotinylation during co-expression with Bir A biotin ligase. In addition, both Fe fragments contained the PG-LALA mutations. Both vectors were co-transfected in combination with a Plasmid coding for the BirA biotin ligase in order to get a dimeric CD28-Fc construct with a monovalent biotinylated avi-tag at the C-terminal and of the Fc-knob chain.

Cloning of Targeted CD28 Constructs

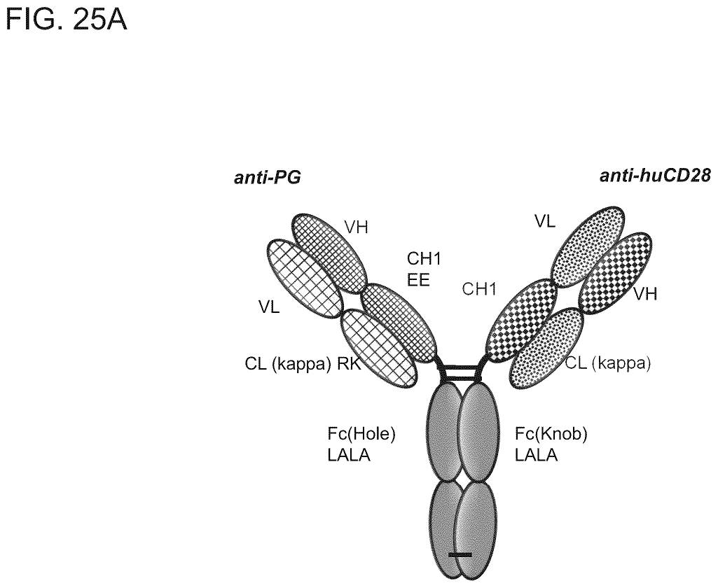

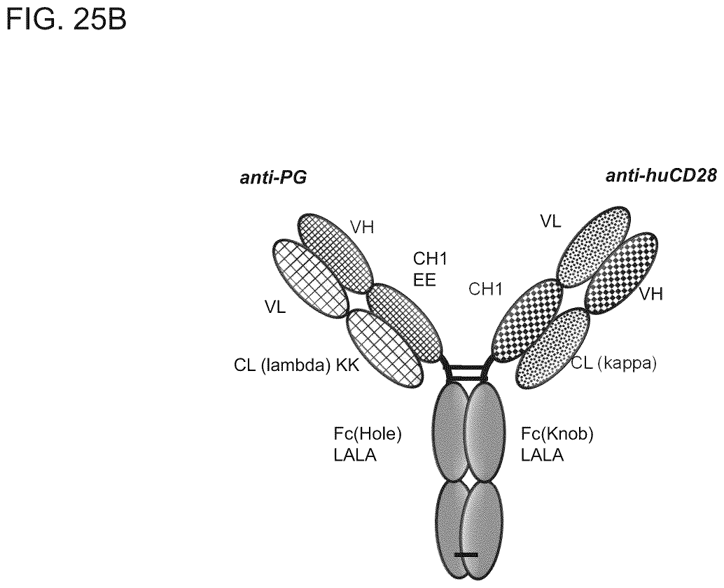

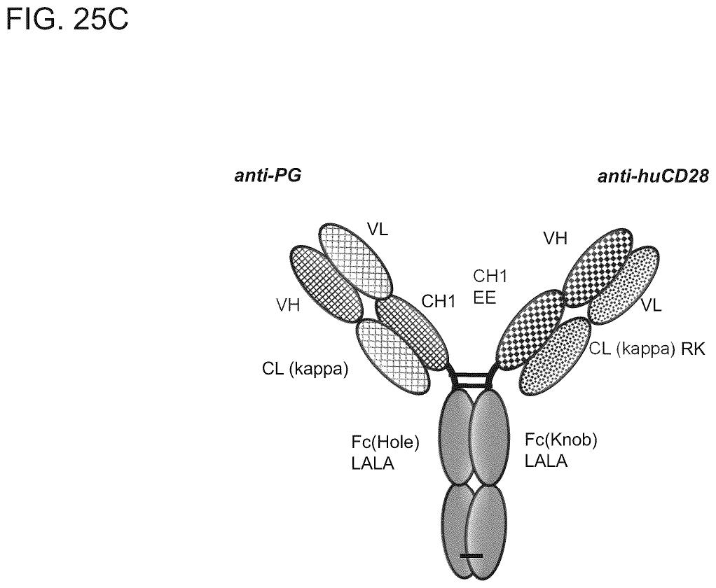

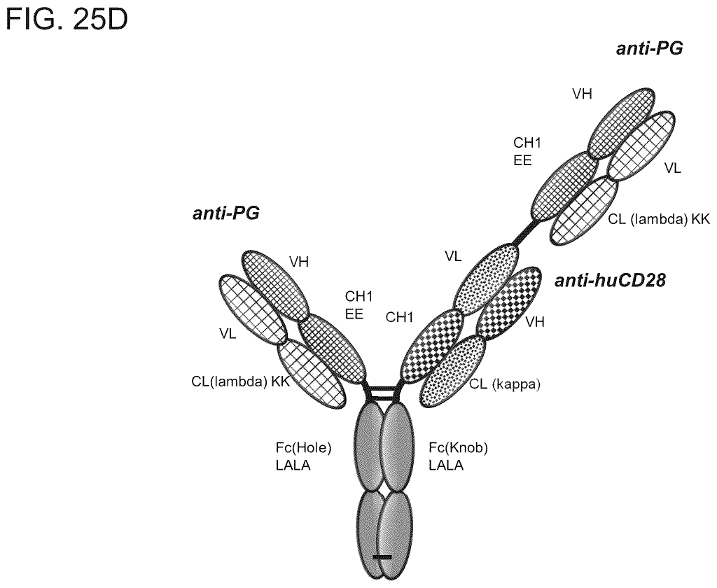

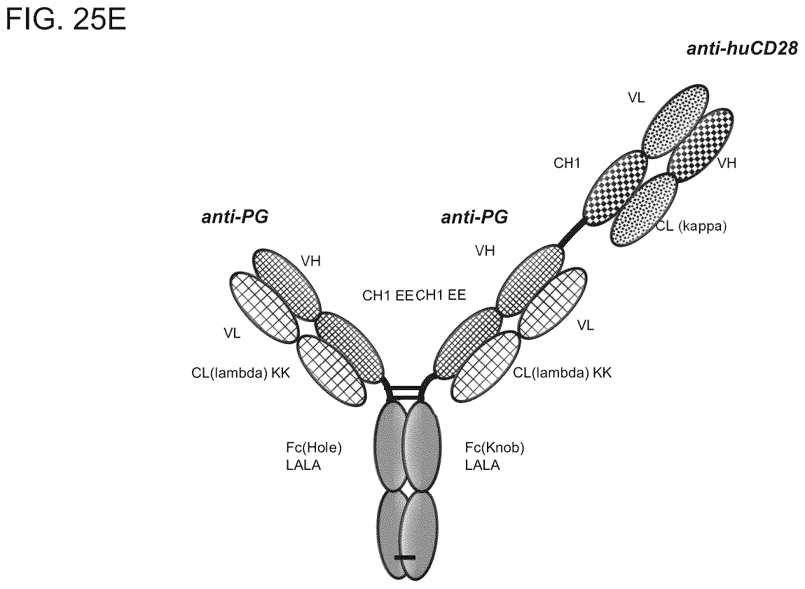

For the generation of the all expression plasmids, the sequences of the variable domains were used and sub-cloned in frame with the respective constant regions which are pre-inserted in the respective recipient mammalian expression vector. A schematic description of the resulting molecules is shown in FIG. 25A-FIG. 25E. Where indicated, Leu234Ala and Leu235Ala mutations (LALA) have been introduced in the constant region of the human IgG1 heavy chains to abrogate binding to Fc gamma receptors. For the generation of bi- and tri-specific antibodies, Fc-fragments contained either the “knob” or “hole” mutations to avoid mispairing of the heavy chains. In order to avoid mispairing of light chains in all constructs, exchange of VH/VL, CH1/CL(kappa), or CH1/CL(lambda) domains was introduced in one binding moiety (CrossFab technology). In another binding moiety, charges were introduced into the CH1 and Ckappa or Clambda domains.

The following molecules were cloned; a schematic illustration thereof is shown in FIG. 25A-FIG. 25E:

Molecule A, Anti-P329G (M-1.7.24)×CD28 (TGN1412_variant 15_crossed) 1+1, bispecific huIgG1 LALA CrossFab molecule with charge modifications in anti-P329G binder M-1.7.24 and VH/VL exchange in the TGN1412 binder variant 15 (knob) (FIG. 25A, SEQ ID NO: 93, 106, 88, 107).

Molecule B, Anti-P329G (M-1.7.24)×CD28 (TGN1412_variant 8_crossed) 1+1, bispecific huIgG1 LALA CrossFab molecule with charge modifications in anti-P329G binder M-1.7.24 and VH/VL exchange in the TGN1412 binder variant 8 (knob) (FIG. 25A, SEQ ID NO: 93, 108, 88, 109).

Molecule C, Anti-P329G (VH3×VL1)×CD28 (TGN1412_variant 8_crossed) 1+1, bispecific huIgG1 LALA CrossFab molecule with charge modifications in the humanized anti-P329G binder VH3×VL1 and VH/VL exchange in the TGN1412 binder variant 8 (knob) (FIG. 25B, SEQ ID NO 89, 108, 90, 109).

Molecule D, Anti-P329G (VH3VL1)×CD28 (TGN1412_variant 8) 1+1, bispecific huIgG1 LALA CrossFab molecule with charge modifications in the TGN1412 binder variant 8 (knob) and VH/VL exchange in the humanized anti-P329G binder VH3×VL1 (hole) (FIG. 25C, SEQ ID NO:110, 111, 112, 113).

Molecule E, Anti-P329G (VH3VL1)×CD28 (TGN1412_variant 8) 2+1_inverted, 2+1 huIgG1 LALA CrossFab molecule, “inverted orientation” with VH/VL exchange in the TGN1412 binder variant 8 and charge modification in the humanized anti-P329G binder VH3VL1 (FIG. 25D, SEQ ID NO: 89, 108, 90, 114).

Molecule F, Anti-P329G (VH3VL1)×CD28 (TGN1412_variant 8) 2+1_classic, 2+1 huIgG1 LALA CrossFab molecule, “classic orientation” with VH/VL exchange in the TGN1412 binder variant and charge modification in the humanized anti-P329G binder VH3VL1 (FIG. 25E, SEQ ID NO: 89, 108, 90, 115).

Production of Targeted CD28 Constructs

Expression of the above-mentioned molecules is driven by a CMV promoter. Polyadenylation is initiated by a synthetic polyA signal sequence located at the 3′ end of the CDS. In addition, each vector contains an EBV OriP sequence for autosomal replication.

Antibodies and bispecific antibodies were generated by transient transfection of HEK293 EBNA cells or CHO EBNA cells as described above and proteins were purified from the harvested supernatant by standard methods as indicated below.

Purification of Targeted CD28 Constructs

Proteins were purified from filtered cell culture supernatants referring to standard protocols and as described above.

Analytics of Targeted CD28 Constructs

The concentrations of purified proteins were determined as described above. A summary of the purification parameters of all molecules is given in Table 19.

TABLE 19
Summary of the production and purification of the
“Anti-P329G (M-1.7.24) × CD28 (TGN1412 _variant
15_crossed) 1 + 1” molecule

| | | | Analytical SEC | Purity |
|---|---|---|---|---|
| | | | (HMW/Mono- | measured by |
| Mol- | | Yield | mer/LMW) | CE-SDS |
| ecule | Description | [mg/l] | [%] | [%] |
| A | Anti-P329G | 7.15 | 0/95.81/4.19 | 95.33 |
| | (M-1.7.24) × CD28 | | | |
| | (TGN1412_variant | | | |
| | 15_crossed) 1 + 1 | | | |

SPR Analysis of PG-Targeted CD28 Molecule A

Samples

Samples described in Table 20 were analyzed for binding to human CD28-Fc and to constructs with human Fc bearing the P329G mutation. PGP-32

TABLE 20
Description of the samples analyzed for binding
to human CD28-Fc and human Fc (P329G).

| Binder | TAPIR ID | Format |
|---|---|---|
| anti-P329G (M-1.7.24) × CD28 | P1AE9465-005 | Bispecific |
| (TGN1412_var15_crossed) 1 + 1 | | (immobilized |
| | | or analyte) |
| human CD28-Fc biotinylated | P1AE1329-007 | Homodimer of |
| | | CD28 fused to |
| | | Fc (immobilized) |
| TCB (P329G) | P1AE7925-003 | Unrelated TCB |
| | | with P329G |
| | | mutation on Fc |
| | | (analyte) |

Affinity of Anti-P329G (M-1.7.24)×CD28 (TGN1412 Var15 Crossed) 1+1 to Human CD28-Fc
Instrumentation: Biacore T200
Chip: C1 (#782)
Fc4: anti-human Fe specific (Roche internal)
Capture: 25 nM CD28-Fc (P1AE1329-007) for 30 s
Analyte: anti-P329G (M-1.7.24)×CD28 (TGN1412_var15_crossed) 1+1 (P1AE9465-005)
Running buffer: HBS-EP
T°: 25° C.
Dilution: 3-fold dilution in HBS-EP from 0.09 to 200 nM
Flow: 30 μl/min
Association: 240 sec
Dissociation: 1000 sec
Regeneration: 10 mM glycine pH 2.1 for 2×60 sec

SPR experiments were performed on a Biacore T200 with HBS-EP+ as running buffer (0.01 M HEPES pH 7.4, 0.15 M NaCl, 0.005% Surfactant P20 (BR-1006-69, GE Healthcare)). Anti-human Fc specific antibodies (Roche internal) were directly immobilized by amine coupling on a C1 chip (GE Healthcare). CD28-Fc was captured for 30 s at 25 nM. A three-fold dilution series of the anti-P329G (M-1.7.24)×CD28 (TGN1412_var15_crossed) 1+1 was passed over the ligand at 30 μl/min for 240 sec to record the association phase. The dissociation phase was monitored for 1000 s and triggered by switching from the sample solution to HBS-EP+. The chip surface was regenerated after every cycle using two injections of 10 mM glycine pH 2.1 for 60 sec. Bulk refractive index differences were corrected for by subtracting the response obtained on the reference flow cell 1. The affinity constants were derived from the kinetic rate constants by fitting to a 1:1 Langmuir binding using the Biaeval software (GE Healthcare). The measure was performed in triplicate with independent dilution series. The kinetic constants for a 1:1 Langmuir binding are summarized in Table 21.

TABLE 21
Kinetic constants (1:1 Langmuir binding) of the interaction between anti-P329G
(M-1.7.24) × CD28 (TGN1412_var15_crossed) 1 + 1 and human CD28-Fc. Average and standard
deviation (in parenthesis) of independent triplicate (independent dilutions series within the same run).

| Binder | TAPIR ID | ka (1/Ms) | kd (1/s) | KD (M) | Rmax (RU) |
|---|---|---|---|---|---|
| anti-P329G (M-1.7.24) × CD28 | P1AE9465-005 | 1.56E+06 | 5.00E−03 | 3.22E−09 | 22 |
| (TGN1412_var15_crossed) 1 + 1 | | (1.40E+05) | (2.39E−04) | (1.6E−10) | (0.8) |

Affinity of Anti-P329G (M-1.7.24)×CD28 (TGN1412 Var15 Crossed) 1+1 to TCB (P329G)
Instrumentation: Biacore T200
Chip: C1 (#787)
Fc 3: anti-P329G (M-1.7.24)×CD28 (TGN1412_var15_crossed) 1+1 (P1AE9465-005)
Analyte: unrelated TCB with P329G on Fc (P1AE7925-003)
Running buffer: HBS-EP
T°: 25° C.
Dilution: 3-fold dilution in HBS-EP from 0.69 to 500 nM
Flow: 30 μl/min
Association: 240 sec
Dissociation: 600 sec
Regeneration: 10 mM glycine pH 1.5 for 2×60 sec

SPR experiments were performed on a Biacore T200 with HBS-EP+ as running buffer (0.01 M HEPES pH 7.4, 0.15 M NaCl, 0.005% Surfactant P20 (BR-1006-69, GE Healthcare)). Anti-P329G (M-1.7.24)×CD28 (TGN1412_var15_crossed) 1+1 was directly immobilized by amine coupling on a C1 chip (GE Healthcare). A three-fold dilution series of an unrelated TCB with P329G on Fc was passed over the ligand at 30 μl/min for 240 sec to record the association phase. The dissociation phase was monitored for 600 s and triggered by switching from the sample solution to HBS-EP+. The chip surface was regenerated after every cycle using two injections of 10 mM glycine pH 1.5 for 60 sec. Bulk refractive index differences were corrected for by subtracting the response obtained on the reference flow cell 1. The affinity constants were derived from the kinetic rate constants by fitting to a 1:1 Langmuir binding using the Biaeval software (GE Healthcare). The measure was performed in triplicate with independent dilution series. The kinetic constants for a 1:1 Langmuir binding are summarized in Table 22.

TABLE 22
Kinetic constants (1:1 Langmuir binding) of the interaction of anti-P329G
(M-1.7.24) × CD28 (TGN1412_var15_crossed) 1 + 1 with a TCB bearing the
P329G mutation on the Fc. Average and standard deviation (in parenthesis) of
independent triplicate (independent dilutions series within the same run).

| Binder | TAPIR ID | ka (1/Ms) | kd (1/s) | KD (M) | Rmax (RU) |
|---|---|---|---|---|---|
| anti-P329G (M-1.7.24) × CD28 | P1AE9465-005 | 4.24E+05 | 8.83E−03 | 3.96E−08 | 35 |
| (TGN1412_var15_crossed) 1 + 1 | | (5.72E+04) | (2.07E−03) | (9.83E−10) | (1) |

Simultaneous Binding of Anti-P329G (M-1.7.24)×CD28 (TGN1412 Var15 Crossed) 1+1 to Human CD28-Fc and TCB (P329G )
Instrumentation: Biacore T200
Chip: C1 (#787)
Fc 3: anti-P329G (M-1.7.24)×CD28 (TGN1412_var15_crossed) 1+1 (P1AE9465-005)
Analyte: human CD28-Fc (P1AE1329-007) followed by unrelated TCB with P329G on Fc (P1AE7925-003)
Running buffer: HBS-EP
T°: 25° C.
Flow: 30 μl/min

SPR experiments were performed on a Biacore T200 with HBS-EP+ as running buffer (0.01 M HEPES pH 7.4, 0.15 M NaCl, 0.005% Surfactant P20 (BR-1006-69, GE Healthcare)). Anti-P329G (M-1.7.24)×CD28 (TGN1412_var15_crossed) 1+1 was chemically immobilized on a C1 chip using the standard amine coupling kit (GE Healthcare). Six hundred nM of human CD28-Fc were injected at 30 μl/min for 120 see, followed by 500 nM of unrelated TCB with P329G mutation on Fe for 120 sec. The injection was repeated two times.

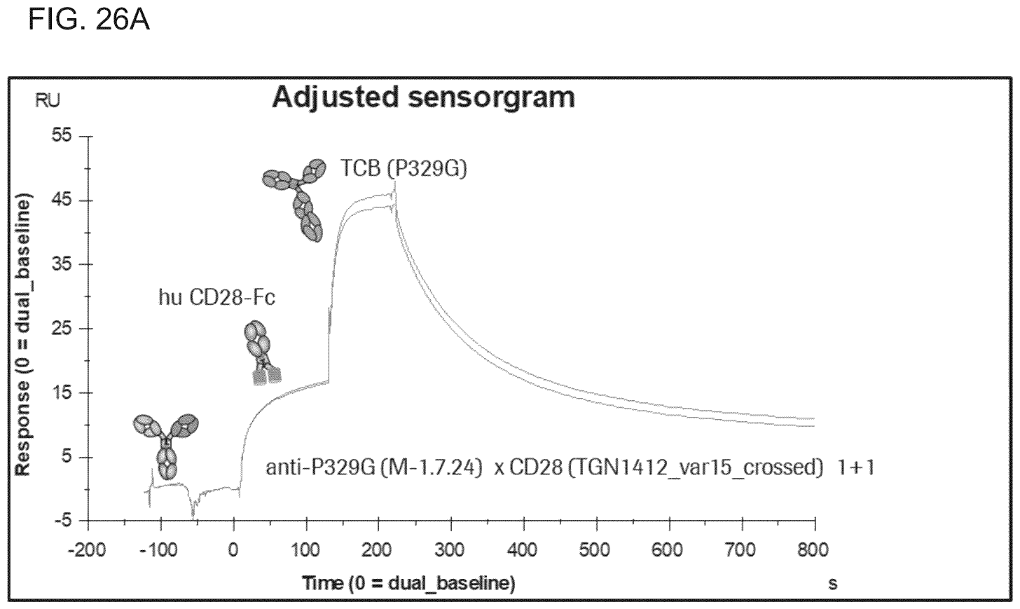

The sensorgram of simultaneous binding is shown in FIG. 26.

Binding of CD28 Bispecific Antibodies to Cells Over-Expressing Human CD28

To measure the binding to human CD28 we performed FACS-based binding assay with CHO cells, that were stably transfected to overexpress human CD28 (parental cell line CHO-k1 ATCC #CCL-61).

Briefly, adherent CHO cells were detached using Cell Dissociation Buffer (Gibco), counted and checked for viability. All subsequent steps were performed at 4° C.

Cells were re-suspended in FACS buffer at 1 Mio cells per ml. 0.1 Mio cells were plated per well of a round-bottom 96-well-plate, centrifuged and supernatants were discarded. Cells were stained in a total volume of 50 ul per well and increasing concentrations of the indicated CD28 bispecific molecule (0.12-500 nM) for 30 minutes at 4° C. Cells were washed twice with FACS buffer and incubated for 30 min at 4° C. in a total of 25 ul per well, containing the pre-diluted secondary antibody (PE-AffiniPure F(ab′)2 Fragment Goat Anti-Human IgG, Fey Fragment Specific from Jackson Immunoresearch, 109-116-170), diluted 1:100 in FACS buffer. Cells were washed twice and analyzed on a BD Canto flow cytometer, equipped with the software FACS Diva. Binding curves and EC50 values were obtained using GraphPadPrism6.

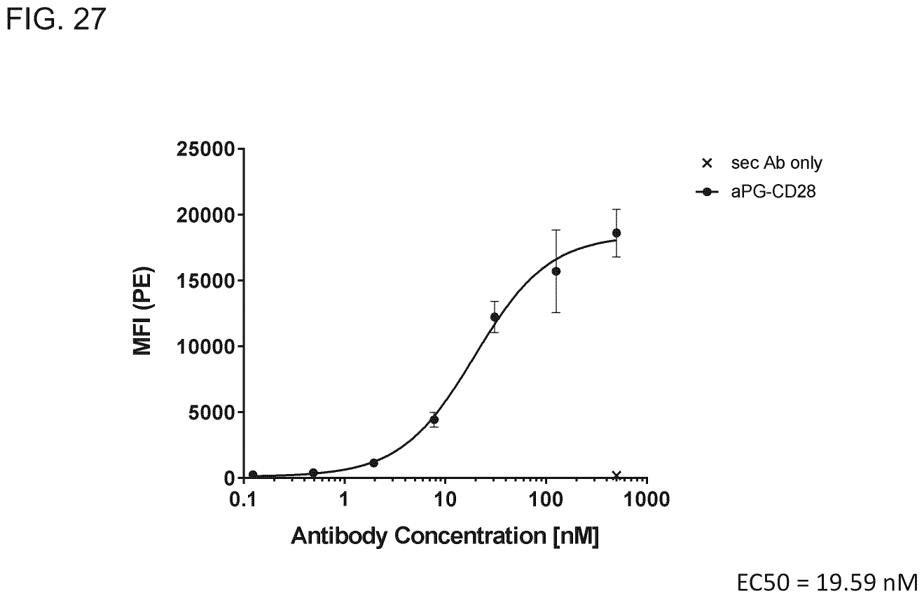

FIG. 27 shows concentration-dependent binding of the aPG-CD28 molecule to human CD28 with an EC50 of 19.6 nM.

IL-2 Reporter Assay (Functional Characterization of T-Cell Activation)

To assess the ability of aPG-CD28 to support CD3 IgG-mediated T cell activation, IL-2 reporter cells (Promega, Ca No J1651) were used as effector cells (Jurkat T cell line that expresses a luciferase reporter driven by the IL-2 promoter).

Briefly, 2.5×10⁴ IL-2 reporter cells were added into wells of a white flat-bottom 384 well plate (353988 Falcon™) and incubated in presence of 625 pM CD3 IgG (CD3 binder CH2527 with PGLALA-containing Fe part) alone or in combination with decreasing concentrations of the CD28 bispecific molecules (34.4 nM-8.4 pM; 1:4 dilution steps) for 4 h at 37° C. The assay plate was incubated at room temperature for 5 min, followed by addition of 20 ul of substrate (ONE-Glo solution, Promega, Cat No E6120). After another 10 min of incubation at room temperature in the dark, Luminescence (counts/sec) was quantified using a Tecan Spark 10M plate reader.

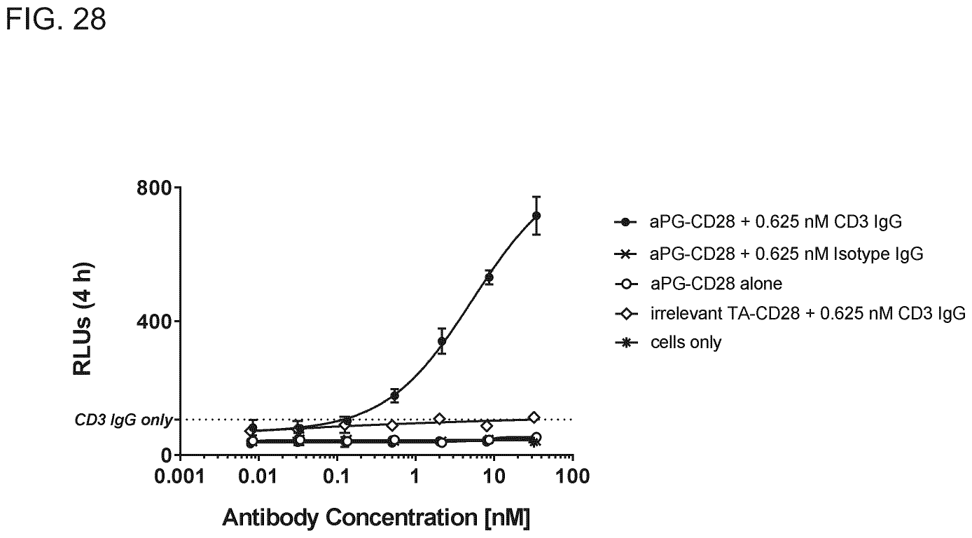

As depicted in FIG. 28, the PG-targeted CD28 molecule is not able to induce any Jurkat activation in the absence of the first T-cell activation stimulus, namely the CD3 IgG. However, upon simultaneous binding to the PG mutation of the Fc part of the CD3 IgG, as well as to CD28 expressed on Jurkt cells, it is able to boost CD3 IgG-induced baseline activation of Jurkat cells in a concentration-dependent manner. Thereby, the strongest boosting effect is observed at 34.4 nM of the aPG-CD28 molecule. The effect depends on crosslinking of the PG-CD28 via both targeting moieties: no boosting of CD3 IgG-mediated Jurkat activation was observed in presence of a similar CD28 molecule exhibiting an unrelated tumor-antigen targeting moiety instead of the PG-targeting one. Moreover, the PG-targeted CD28 molecule is not able to induce any Jurkat T cell activation when administered either alone or in presence of a human IgG isotype control, containing the PGLALA in its Fc part.

Conclusion

The anti-P329G (M-1.7.24)×CD28 (TGN1412_var15_crossed) 1+1 is binding to human CD28-Fc as expected with an affinity of 3 nM. The affinity to Fc (P329G) is lower than expected and is around 40 nM instead of 5 nM. The affinity might be influenced by the CD28 binding Fab arm. Both antigens can be bound at the same time, as expected from a bispecific molecule.

The anti-P329G-anti-CD28 molecule binds to the P329G mutation, as well as to human CD28 in a concentration-dependent manner and consequently, induces (Jurkat) T cell activation in presence of a first T-cell stimulus only.

### Example 5

### Fc Binding Molecules Comprising a Cytokine (IL2v) Immune Activating Moiety

The following molecule was prepared in this example by transfection in mammalian cells and purification by ProteinA affinity chromatography and size exclusion chromatography.

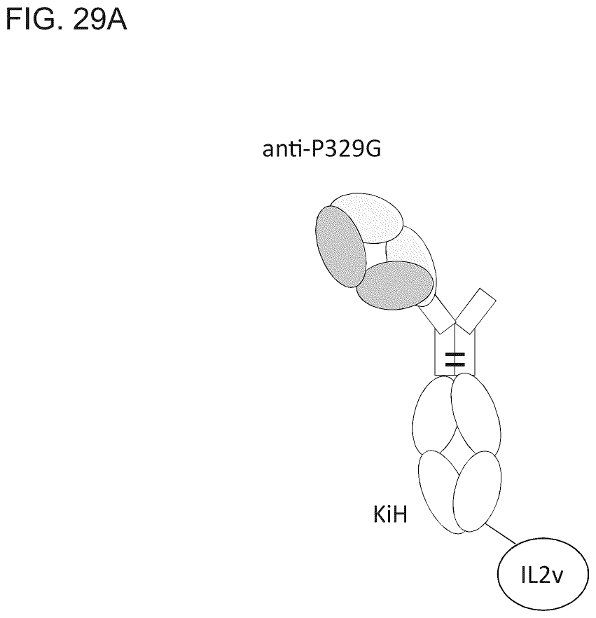

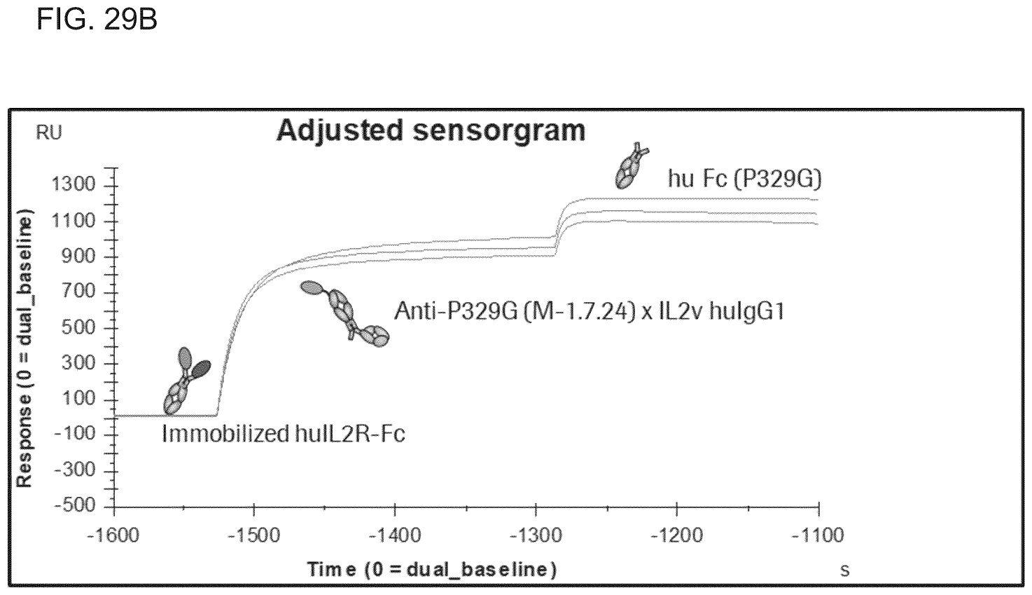

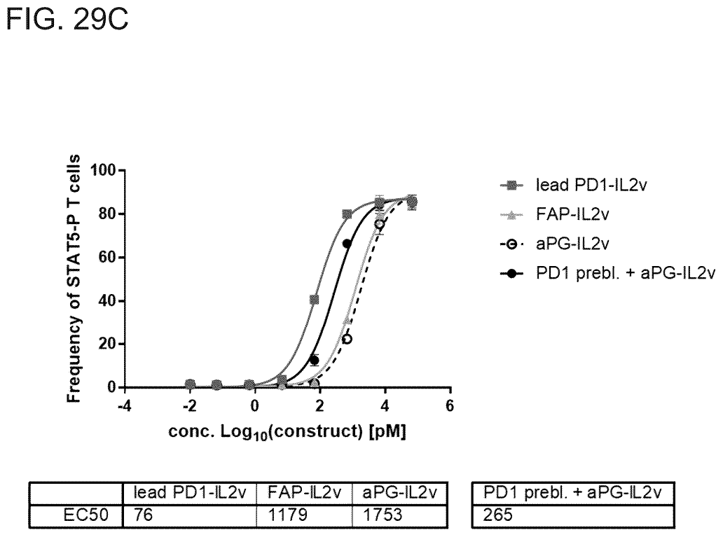

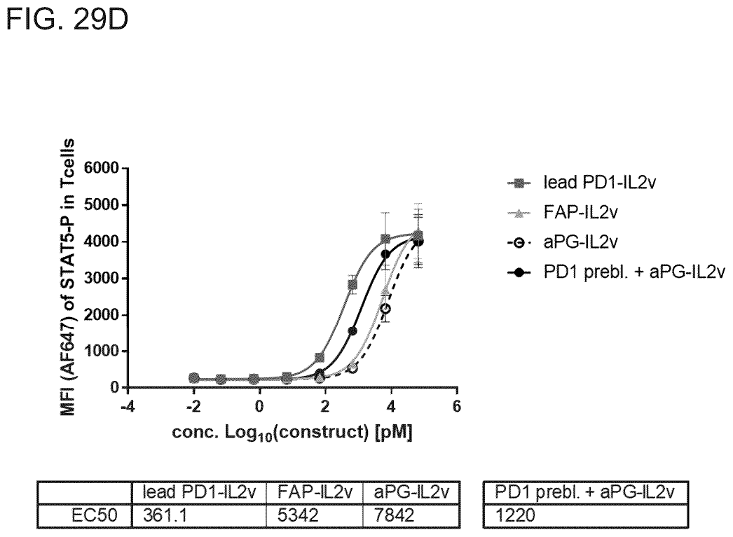

Anti-P329G (M-1.7.24)×IL2v huIgG1 (format of FIG. 29A, SEQ ID NOs 86, 116, 88).

Anti-P329G (VH3VL1)×IL2v huIgG1 (format of FIG. 29A, SEQ ID NOs 15, 116, 90).

Anti-P329G (M-1.7.24)×IL2v huIgG1 was produced in good quality (Table 23).

TABLE 23
Biochemical analysis of Anti-P329G (M-1.7.24) ×
IL2v huIgG1. Monomer content determined by analytical
size exclusion chromatography. Purity determined
by non-reducing SDS capillary electrophoresis.

| | Monomer | Purity |
|---|---|---|
| Molecule | [%] | [%] |
| Anti-P329G (M-1.7.24) × IL2v huIgG1 | 100 | 100 |

Samples

Following samples were analyzed for binding to human IL2R beta-gamma-Fc and to constructs with human Fc bearing the P329G mutation (Table 24).

TABLE 24
Description of the samples analyzed for binding
to human IL2R beta-gamma-Fc and human Fc (P329G).

| Binder | TAPIR ID | Format |
|---|---|---|
| anti-P329G (M-1.7.24) × | P1AF1749-003 | IL2 fusion to one-armed IgG |
| IL2v huIgG1 | | |
| human IL2R beta- | P1AE2657-005 | Heterodimer of IL2R beta |
| gamma-Fc biotinylated | | and IL2R gamma chain fused |
| | | to Fc (analyte) |
| TCB (P329G) | P1AE7925-003 | Unrelated TCB with P329G |
| | | mutation on Fc (analyte) |
| human Fc (P329G) | P1AD9000-004 | Fc part of human IgG with |
| | | P329G mutation (analyte) |

Human Fc (P329G) was prepared by plasmin digestion of a human IgG1 followed by affinity purification by ProteinA and size exclusion chromatography.

Affinity of Anti-P329G (M-1.7.24)×IL2v huIgG1 to Human IL2R-Fc
Instrumentation: Biacore T200
Chip: CM5 (#772)
Fc1 to 4: anti-human Fab specific (GE Healthcare 28-9583-25)
Capture: 10 nM IgG-IL2v for 120 s
Analyte: human IL2R beta-gamma-Fc (P1AE2657-005)
Running buffer: HBS-EP
T°: 25° C.
Dilution: 3-fold dilution in HBS-EP from 0.09 to 200 nM
Flow: 30 μl/min
Association: 240 sec
Dissociation: 800 sec
Regeneration: 10 mM glycine pH 2.1 for 2×60 sec

SPR experiments were performed on a Biacore T200 with HBS-EP+ as running buffer (0.01 M HEPES pH 7.4, 0.15 M NaCl, 0.005% Surfactant P20 (BR-1006-69, GE Healthcare)). Anti-human Fab specific antibodies (GE Healthcare 28-9583-25) were directly immobilized by amine coupling on a CM5 chip (GE Healthcare). The anti-P329G (M-1.7.24)×IL2v huIgG1 was captured for 120 s at 10 nM. A three-fold dilution series of the human IL2R beta-gamma-Fc was passed over the ligand at 30 l/min for 240 sec to record the association phase. The dissociation phase was monitored for 800 s and triggered by switching from the sample solution to HBS-EP+. The chip surface was regenerated after every cycle using two injections of 10 mM glycine pH 2.1 for 60 sec. Bulk refractive index differences were corrected for by subtracting the response obtained on the reference flow cell 1. The affinity constants were derived from the kinetic rate constants by fitting to a 1:1 Langmuir binding using the Biaeval software (GE Healthcare). The measure was performed in triplicate with independent dilution series.

The kinetic constants for a 1:1 Langmuir binding are summarized in Table 25.

TABLE 25
Kinetic constants (1:1 Langmuir binding) of the interaction between anti-P329G
(M-1.7.24) × IL2v huIgG1 and human IL2R-Fc. Average and standard deviation (in
parenthesis) of independent triplicate (independent dilutions series within the same run).

| Binder | TAPIR ID | ka (1/Ms) | kd (1/s) | KD (M) | Rmax (RU) |
|---|---|---|---|---|---|
| anti-P329G (M-1.7.24) × | P1AF1749-003 | 1.09E+05 | 1.50E−04 | 1.38E−09 | 8.6 |
| IL2v huIgG1) | | (2.52E+03) | (1.12E−05) | (1.1E−10) | (0.4) |

Affinity of Anti-P329G (M-1.7.24)×IL2v huIgG1 to TCB (P329G)
Instrumentation: Biacore T200
Chip: C1 (#785)
Fc 2: anti-P329G (M-1.7.24)×IL2v huIgG1
Analyte: unrelated TCB with P329G on Fe (P1AE7925-003)
Running buffer: HBS-EP
T°: 25° C.
Dilution: 3-fold dilution in HBS-EP from 0.09 to 600 nM
Flow: 30 μl/min
Association: 240 sec
Dissociation: 800 sec
Regeneration: 10 mM glycine pH 1.5 for 2×60 sec

SPR experiments were performed on a Biacore T200 with HBS-EP+ as running buffer (0.01 M HEPES pH 7.4, 0.15 M NaCl, 0.005% Surfactant P20 (BR-1006-69, GE Healthcare)). Anti-P329G (M-1.7.24)×IL2v huIgG1 was directly immobilized by amine coupling on a C1 chip (GE Healthcare). A three-fold dilution series of an unrelated TCB with P329G on Fc was passed over the ligand at 30 μl/min for 240 sec to record the association phase. The dissociation phase was monitored for 800 s and triggered by switching from the sample solution to HBS-EP+. The chip surface was regenerated after every cycle using two injections of 10 mM glycine pH 1.5 for 60 sec. Bulk refractive index differences were corrected for by subtracting the response obtained on the reference flow cell 1. The affinity constants were derived from the kinetic rate constants by fitting to a 1:1 Langmuir binding using the Biaeval software (GE Healthcare). The measure was performed in triplicate with independent dilution series.

The kinetic constants for a 1:1 Langmuir binding are summarized in Table 26.

TABLE 26
Kinetic constants (1:1 Langmuir binding) of the interaction of anti-P329G (M-1.7.24) ×
IL2v huIgG1 with a TCB bearing the P329G mutation on the Fc. Average and standard deviation
(in parenthesis) of independent triplicate (independent dilutions series within the same run).

| Binder | TAPIR ID | ka (1/Ms) | kd (1/s) | KD (M) | Rmax (RU) |
|---|---|---|---|---|---|
| anti-P329G (M-1.7.24) × | P1AF1749-003 | 9.43E+05 | 3.98E−03 | 4.25E−09 | 144 |
| IL2v huIgG1) | | (9.54E+04) | (7.51E−06) | (4.03E−10) | (3) |

Simultaneous Binding of Anti-P329G (M-1.7.24)×IL2v huIgG1 to Human IL2R Beta-Gamma-Fc and Human Fc (P329G)
Instrumentation: Biacore T200
Chip: SA (#783)
Fc 2: IL2R beta-gamma-Fc biotinylated (P1AE2657-005)
Analyte: anti-P329G (M-1.7.24)×IL2v huIgG1, followed by human Fe (P329G) (P1AD9000-004)
Running buffer: HBS-EP
T°: 25° C.
Flow: 30 μl/min

SPR experiments were performed on a Biacore T200 with HBS-EP+ as running buffer (0.01 M HEPES pH 7.4, 0.15 M NaCl, 0.005% Surfactant P20 (BR-1006-69, GE Healthcare)). Biotinylated human IL2R beta-gamma-Fc was immobilized on a streptavidin chip. Two hundred nM of anti-P329G (M-1.7.24)×IL2v huIgG1 were injected at 30 μl/min for 240 see, followed by 400 nM of human Fe (P329G) for 180 sec. The injection was repeated three times.

The sensorgram of simultaneous binding is shown in FIG. 29B.

Conclusion

The anti-P329G (M-1.7.24)×IL2v huIgG1 is binding to human IL2R beta-gamma-Fc and to human Fe (P329G) as expected. Both antigens can be bound at the same time, as expected from a bispecific molecule.

IL-2R Signaling (STAT5-P) Upon Treatment with Increasing Doses of aPG-IL2v on Anti-PD-1 Treated and Untreated PD-1⁺ CD4 T Cells

In this experiment, the STAT5 phosphorylation (STAT5-P) was used to demonstrate the IL-2v delivery of the anti-PG-IL2v compound to the IL-2R of PD-1⁺ CD4 T cells previously pre-treated with anti-PD-1 antibody.

For this purpose CD4 T cells were sorted from healthy donor PBMCs with CD4 beads (130-045-101, Miltenyi) and activated for 3 days in presence of 1 pg/ml plate bound anti-CD3 (overnight pre-coated, clone OKT3, #317315, BioLegend) and 1 pg/ml of soluble anti-CD28 (clone CD28.2, #302923, BioLegend) antibodies to induce PD-1 expression. Three days later, the cells were harvested and washed several times to remove endogenous IL-2. Then, the cells were divided in two groups, one of which was incubated with saturating concentration of anti-PD1 antibody (in-house molecule, 10 ug/ml) for 30 min at RT.

Following several washing steps to remove the excess unbound anti-PD-1 antibody, the anti-PD1 pre-treated and untreated cells (50 ul, 2*106 cells/ml) were seeded into a V-bottom plate before being treated for 12 min at 37° C. with increasing concentrations of treatment antibodies (50 ul, 1:10 dilution steps with the top concentration of 66 nM ). To preserve the phosphorylation state, an equal amount of Phosphoflow Fix Buffer I (100 ul, 557870, BD) was added right after 12 minutes incubation with the various constructs. The cells were then incubated for additional 30 min at 37° C. before being permeabilized overnight at −80° C. with Phosphoflow PermBuffer III (558050, BD). On the next day STAT-5 in its phosphorylated form was stained for 30 min at 4° C. by using an anti-STAT-5P antibody (47/Stat5(pY694) clone, 562076, BD).

The cells were acquired at the FACS BD-LSR Fortessa (BD Bioscience). The frequency and the geometric mean of fluorescent intensity (MFI) of STAT-5P were determined with FlowJo (V10) and plotted with GraphPad Prism (FIG. 29C and FIG. 29D).

This experiment shows that the aPG-IL2v delivers IL-2v to anti-PD-1 pre-treated CD4 T cells with roughly 3 fold less potency than PD1-IL2v, used here as positive control. In absence of anti-PD-1 pretreatment, and therefore of targeting, PG-IL2v was comparable to FAP-IL2v, used here as control for assessing untargeted IL-2R signaling.

### Example 6

### Fc Binding Molecules Comprising Costimulatory (4-1BBL) Immune Activating Moiety

Preparation of P329G-targeted split trimeric 4-1BB ligand Fc fusion protein The variable region of heavy and light chain DNA sequences encoding a binder specific for the P329G Fc mutation, were subcloned in frame with either the constant heavy chain of the hole or the constant light chain of human IgG1.

The DNA sequence encoding part of the ectodomain (amino acid 71-248) of human 4-1BB ligand was synthetized according to the P41273 sequence of Uniprot database.

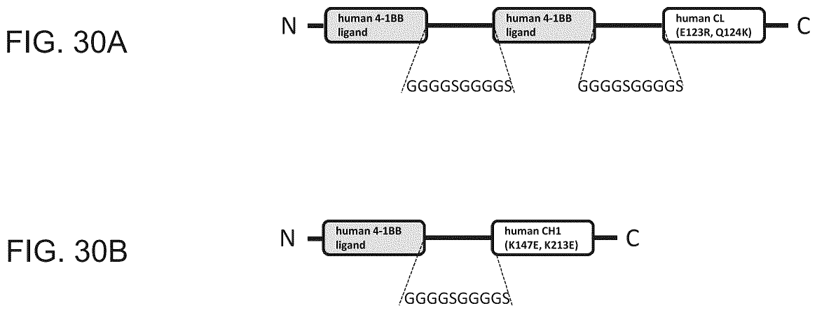

A polypeptide containing two ectodomains of 4-1BB ligand, separated by (G4S)2 linkers, and fused to the human IgG1-CL domain, was cloned as depicted in FIG. 30A: human 4-1BB ligand, (G4S)2 connector, human 4-1BB ligand, (G4S)2 connector, human CL.

A polypeptide containing one ectodomain of 4-1BB ligand and fused to the human IgG1-CH domain, was cloned as described in FIG. 30B: human 4-1BB ligand, (G4S)2 connector, human CH.

To improve correct pairing the following mutations have been introduced in the crossed CH-CL. In the dimeric 4-1BB ligand fused to human CL, E123R and Q124K. In the monomeric 4-1BB ligand fused to human CH1, K147E and K213E.

The Leu234Ala and Leu235Ala mutations have been introduced in the constant region of the knob and hole heavy chains to abrogate binding to Fc gamma receptors.

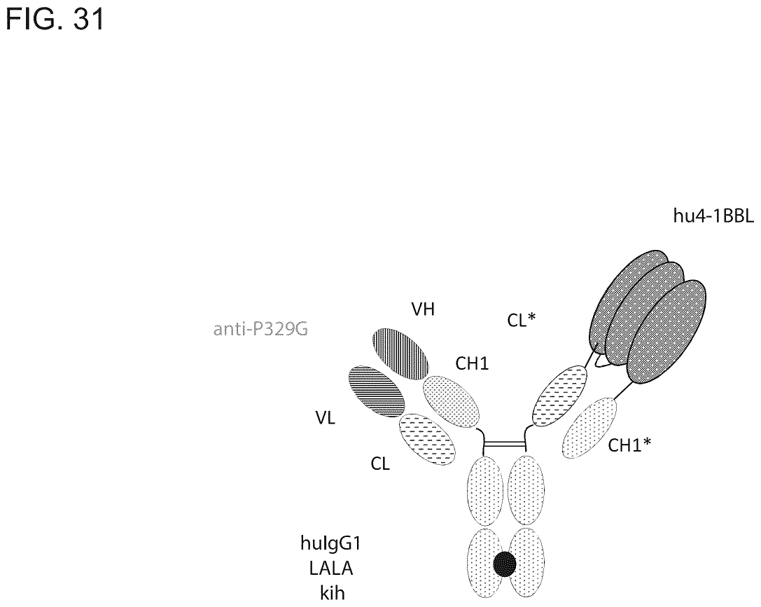

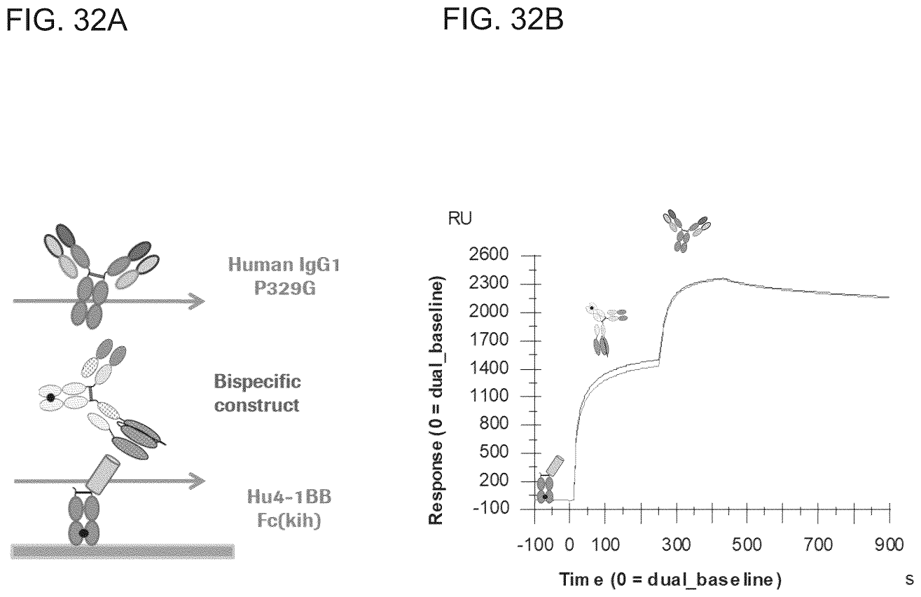

Combination of the dimeric ligand-Fc knob chain containing the S354C/T366W mutations, the monomeric CH1 fusion, the targeted anti-P329G-Fc hole chain containing the Y349C/T366S/L368A/Y407V mutations and the anti-P329G light chain allows generation of a heterodimer, which includes an assembled trimeric 4-1BB ligand and a P329G binding Fab, also termed anti-P329G×4-1BBL huIgG1 (FIG. 31).

The following molecules were cloned; a schematic illustration thereof is shown in FIG. 31:

Anti-P329G (M-1.7.24)×4-1BBL huIgG1 LALA with charge modifications in anti-P329G binder (SEQ ID NO: 10, 129, 130, 131).

Humanized anti-P329G(VH3VL1)×4-1BBL huIgG1 LALA with charge modifications in anti-P329G binder (SEQ ID NO: 15, 129, 130, 132).

The bispecific constructs were produced by transfecting mammalian cells with the corresponding expression vectors in a 1:1:1:1 (“vector 4-1BBL Fc-knob chain”: “vector 4-1BBL light chain” “vector Fc-hole chain”: “vector light chain”).

Production of IgG-Like Proteins in HEK293 EBNA or CHO EBNA Cells

The bispecific antibodies were generated by transient transfection of HEK293 EBNA cells or CHO EBNA cells as described above and proteins were purified from the harvested supernatant by standard methods as indicated below.

Purification of IgG-Like Proteins

Proteins were purified from filtered cell culture supernatants referring to standard protocols as described above.

Analytics of IgG-Like Proteins

The concentrations of purified proteins were determined as described above.

TABLE 27
Biochemical analysis of anti-P329G (M-1.7.24) × 4-1BBL huIgG1

| | Monomer | Yield | CE-SDS |
|---|---|---|---|
| Molecule | [%] | [mg/l] | (non-red) |
| anti-P329G(M-1.7.24) × | 99 | 6.5 | 97 |
| 4-1BBL huIgG1 | | | |

Functional Characterization of P329G Targeted Split Trimeric 4-1BB Ligand Fc Fusion by Surface Plasmon Resonance

The capacity to bind simultaneously human 4-1BB Fc(kih) and a human Fc containing the P329G mutation, was assessed by surface plasmon resonance (SPR). All SPR experiments were performed on a Biacore T200 at 25° C. with HBS-EP as running buffer (0.01 M HEPES pH 7.4, 0.15 M NaCl, 3 mM EDTA, 0.005% Surfactant P20, Biacore, Freiburg/Germany). Biotinylated human 4-1BB-Fc(kih) protein was directly coupled to a flow cell of a SA chip. Immobilization level of approx. 4000 RU was used.

The anti-P329G(M-1.7.24)×4-1BBL huIgG1 construct was passed at a concentration range of 200 nM with a flow of 30 μL/minute through the flow cells over 240 seconds and dissociation was set to zero sec. An human IgG1 antibody containing the P329G mutation in the Fc was injected as second analyte with a flow of 30 μL/minute through the flow cells over 180 seconds at a concentration of 200 nM (FIG. 32A). The dissociation was monitored for 600 sec. Bulk refractive index differences were corrected for by subtracting the response obtained in a reference flow cell, where no protein was immobilized.

As can be seen in FIG. 32B, anti-P329G(M-1.7.24)×4-1BBL huIgG1 can bind simultaneously human 4-1BB and a human IgG1 containing P329G mutation in the Fc.

Anti-P329G×4-1BBL huIgG1 can Enhance CD20-TCB Mediated T Cell Activation and Tumor Cell Lysis

T cell-mediated lysis by CD20-TCB alone or in combination with anti-P329G(M-1.7.24)×4-1BBL huIgG1 was assessed on WSU DLBCL targets. Human B cell-depleted PBMCs were used as effector cells and the killing as well as T cell activatin was measured after 3 days incubation with the bispecific antibodies (1 nM CD20-TCB, 1 nM anti-P329G×4-1BBL huIgG1).

Briefly, 50000 WSU DLCL2 per well were seeded into a 96-U-bottom plate. Peripheral blood mononuclear cells (PBMCs) were prepared by Histopaque density centrifugation of enriched lymphocyte preparations (buffy coats) obtained from healthy human donors. Fresh blood was diluted with sterile PBS and layered over Histopaque gradient (Sigma, #H8889). After centrifugation (450×g, 30 minutes, no brake, room temperature), the plasma above the PBMC-containing interphase was discarded and PBMCs transferred in a new falcon tube subsequently filled with 50 ml of PBS. The mixture was centrifuged (350×g, 10 minutes, room temperature), the supernatant discarded and the PBMC pellet incubated in erythrocyte lysing solution for 5 min at 37° C. before washing with sterile PBS (centrifugation 300×g, 10 minutes). The resulting PBMC population was resuspended in PBS and counted automatically (ViCell). B cell depletion was performed using CD20 Microbeads (Miltenyi) according to the manufacturer's instructions. B cell depleted PBMCs were counted (ViCell) and resuspended at 5×10⁶/ml in RPMI1640 medium containing 10% FCS and 1% L-alanyl-L-glutamine (Biochrom, K0302). For the killing assay, 250000 B cell-depleted PBMCs were added to the targets (E:T 5:1) and antibody dilutions were added (1 nM end concentration in triplicates). Target cell killing was assessed after 3 days of incubation at 37° C., 5% CO₂ by quantification of LDH released into cell supernatants by apoptotic/necrotic cells (LDH detection kit, Roche Applied Science, #11 644 793 001). Maximal lysis of the target cells (=100%) was achieved by incubation of target cells with 1% Triton X-100. Minimal lysis (=0%) refers to target cells co-incubated with effector cells without bispecific construct. After removal of the supernatatnts for LDH measurement, remaining cells were stained to determine T cell activation by flow cytometry. Briefly, cells were washed twice with PBS, followed by live/dead staining (Zombie Aqua, 20 min at RT). After repeated washing first with PBS followed by FACS Buffer, cells were stained using anti-human CD4-BV605, anti-human CD8-BV711, anti-human CD25-PECy7 and anti-human CD69-BV421 (all Biolegend) for 30 min at 4° C. in the dark. Cells were washed and treates with BD FACS Lysing solution prior to measurement using a BD FACS CantoII.

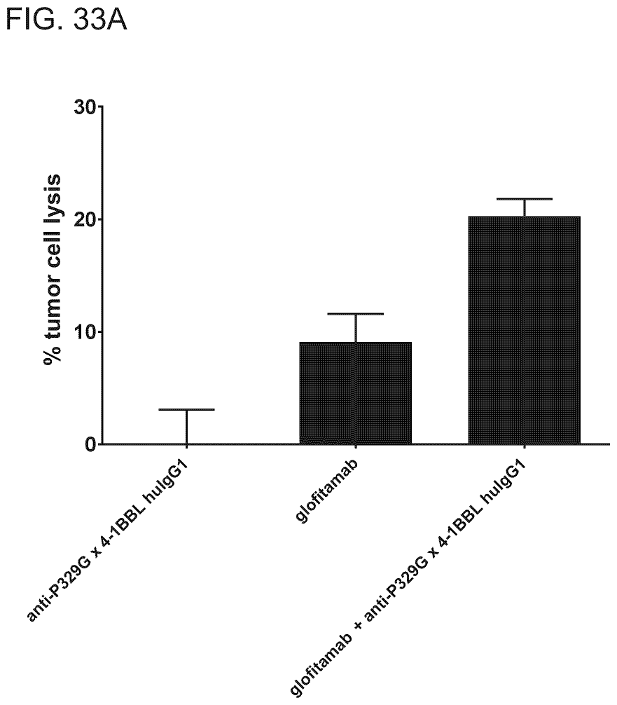

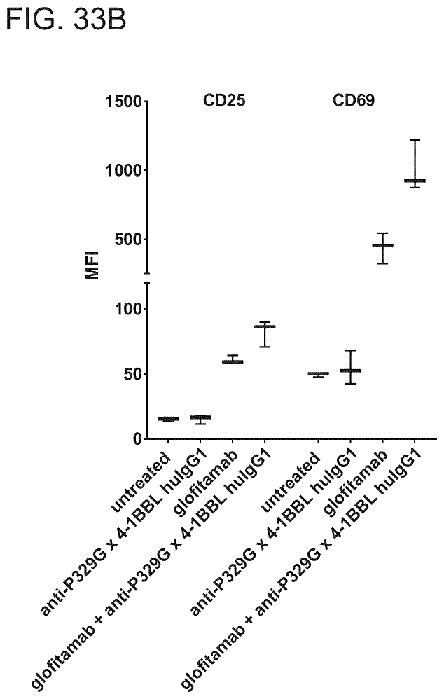

The results show that anti-P329G(M-1.7.24)×4-1BBL huIgG1 can enhance CD20-TCB mediated T cell activation and tumor cell lysis (FIG. 33A-FIG. 33B).

Generation of Bispecific Antibodies with a Bivalent Binding to 4-1BB and a Monovalent Binding to Fc Containing P329G Mutation

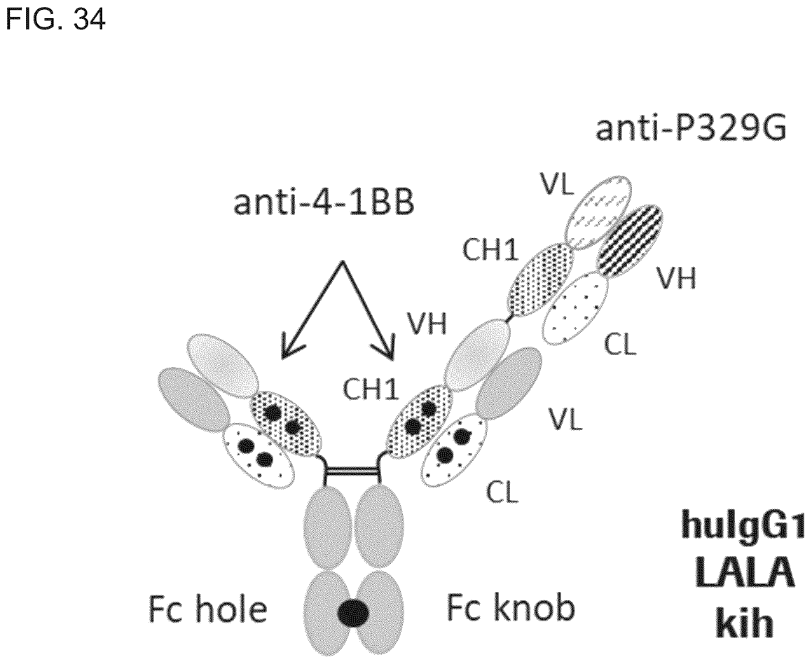

Bispecific agonistic 4-1BB antibodies with bivalent binding for 4-1BB and monovalent binding for Fc containing P329G were prepared as depicted in FIG. 34. This construct is also termed 2+1 anti-4-1BB×anti-P329G huIgG1.

The first heavy chain HC1 of the construct was comprised of the following components: VHCH1 of anti-4-1BB binder (clone 20H4.9), followed by Fc hole. The second heavy chain HC2 was comprised of VLCH1 of anti-P329G binder (in cross Fab format) followed by VHCH1 of an anti-4-1BB (clone 20H4.9) and by Fc knob. The generation and preparation of the P329G binders is described in WO2017/072210, which is incorporated herein by reference. For the 4-1BB binder, the VH and VL sequences of clone 20H4.9 were obtained in accordance with U.S. Pat. No. 7,288,638 B2 or U.S. Pat. No. 7,659,384 B2. Combination of the two heavy chains allows generation of a heterodimer, which includes a P329G binding cross Fab and two 4-1BB binding Fabs (FIG. 34).

To improve correct pairing, the following mutations have been introduced in the CH-CL of the anti-4-1BB Fab molecules: E123R and Q124K in CL and K147E and K213E in CH1. The second light chain LC2 of the anti-P329G binder was composed of VHCL (cross Fab).

The knobs into hole technology was applied by introducing the Y349C/T366S/L368A/Y407V mutations in the first heavy chain HC1 (Fc hole heavy chain) and by introducing the S354C/T366W mutations in the second heavy chain HC2 (Fc knob heavy chain) to allow generation of a heterodimer.

The Leu234Ala and Leu235Ala mutations have been introduced in the constant region of the knob and hole heavy chains to abrogate binding to Fc gamma receptors.

The amino acid sequences for the bispecific antibody 2+1 anti-4-1BB(20H4.9)×anti-P329G(M-1.7.24) huIgG1 can be found in Tables 4. The amino acid sequences for the bispecific antibody 2+1 anti-4-1BB(20H4.9)×anti-P329G(VH3VL1) huIgG1 can be found in Tables 5.

The following molecules were cloned; a schematic illustration thereof is shown in FIG. 34:
Anti-4-1BB(20H4.9)×anti-P329G(M-1.7.24) 2+1, bispecific huIgG1 LALA CrossFab molecule with charge modifications in anti-P329G binder and VH/VL exchange in the anti-P329G binder (knob) (SEQ ID NO: 141, 142, 143, 1442).
Anti-4-1BB(20H4.9)×humanized anti-P329G(VH3VL1) 2+1, bispecific huIgG1 LALA CrossFab molecule with charge modifications in anti-P329G binder and VH/VL exchange in the anti-P329G binder (knob) (SEQ ID NO: 110, 142, 145, 144).

The bispecific constructs were produced by transfecting mammalian cells with the corresponding expression vectors in a 1:1:1:2 (“vector Fc-knob chain”: “vector light chain (a-P329G)”: “vector Fc-hole chain”: “vector light (20H4.9) chain”).

Production of IgG-Like Proteins in HEK293 EBNA or CHO EBNA Cells

The bispecific antibodies were generated by transient transfection of HEK293 EBNA cells or CHO EBNA cells as described above and proteins were purified from the harvested supernatant by standard methods as indicated below.

Purification of IgG-Like Proteins

Proteins were purified from filtered cell culture supernatants referring to standard protocols and as described above.

Analytics of IgG-Like Proteins

The concentrations of purified proteins were determined as described above.

Functional Characterization of Anti-P329G Targeted Split Trimeric 4-1BB Ligand Fc Fusion in In Vitro Assay—NF-κB Activation in Human 4-1BB and NF κB-Luciferase Reporter Gene Expressing Reporter Cell Line Jurkat-Hu4-1BB-NF κB-Luc2

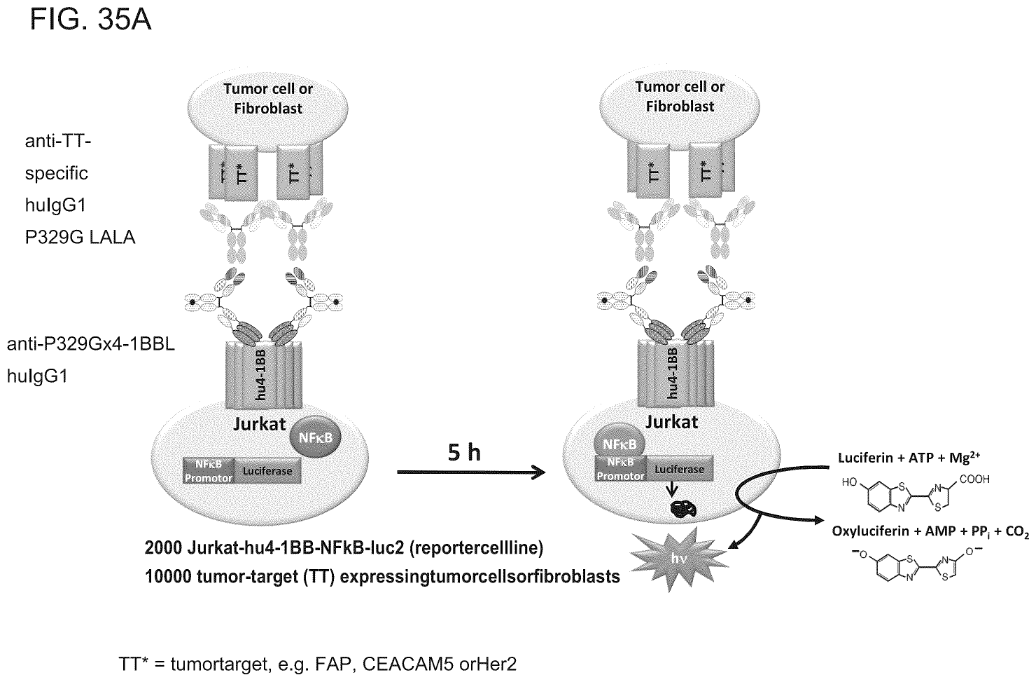

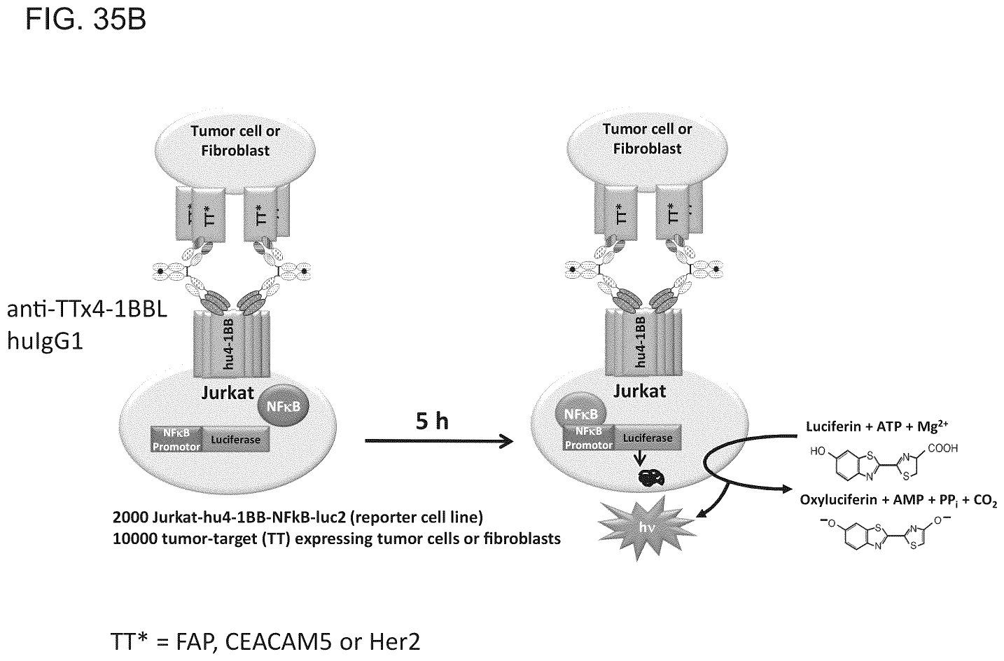

Agonistic binding of the 4-1BB (CD137) receptor to its ligand (4-1BBL) induces 4-1BB-downstream signaling via activation of nuclear factor kappa B (NFkB) and promotes survival and activity of CD8 T cells (Lee H W, Park S J, Choi B K, Kim H H, Nam K O, Kwon B S. 4-1BB promotes the survival of CD8 (+) T lymphocytes by increasing expression of Bcl-x(L) and Bfl-1. J Immunol 2002; 169:4882-4888). To monitor this NFκB-activation mediated by anti-P329G-4-1BBL bispecific antibody-like fusion molecule, Jurkat-hu4-1BB-NFκB-luc2 reporter cell line was purchased from Promega (Germany). The cells were cultured as described above. For the assay, cells were harvested and resuspended in assay medium RPMI 1640 medium supplied with 10% (v/v) FBS and 1% (v/v) GlutaMAX-I. 10 μlcontaining 2×10¹ Jurkat-hu4-1BB-NFκB-luc2 reporter cells were transferred to each well of a sterile white 384-well flat bottom tissue culture plate with lid (Corning, Cat.-No.:3826). Further 10 μL of assay medium alone or containing 1×10⁴ cells of target expressing cells, e.g. CEACAM5 (CD66e)+MKN45 human gastric cancer cells or Her2+ KPL4 human breast cancer or fibroblast activating protein (FAP)+NIH/3T3-huFAP clone 19 genetically modified mouse fibroblast cells were supplied. As positive control 20 μL titrated concentration of tumor-targeted (TT)-4-1BBL were added e.g. Her2 (pertuzumab)-4-1BBL, CEA (T84.66-LCHA)-4-1BBL or FAP (4B9)-4-1BBL. In the other wells 10 μL medium or titrated concentration of anti-P329G-4-1BBL were added. Afterwards 10 μL of titrated concentrations of hulgG P329G LALA were added, whereby they were specific for Her2 (pertuzumab), CEACAM5 (T84.66-LCHA), FAP (4B9) or unspecific (DP47, negative control). The added concentrations of huIgG1 P329G LALA were adjusted to test different ratios between the tumor-specific huIgG1 P329G LALA and anti-P329G-4-1BBL (fixed). The plates were incubated for 5 hours at 37° C. and 5% CO₂ in a cell incubator. 8 μl freshly thawed One-Glo Luciferase assay detection solution (Promega, Cat.-No.: E6110) were added to each well and Luminescence light emission were measured immediately using Tecan microplate reader (500 ms integration time, no filter collecting all wavelength) as units of released light (URLs). Values were baseline corrected using baseline luciferase activity values of untreated wells. The described set ups are schematically shown in FIG. 35.

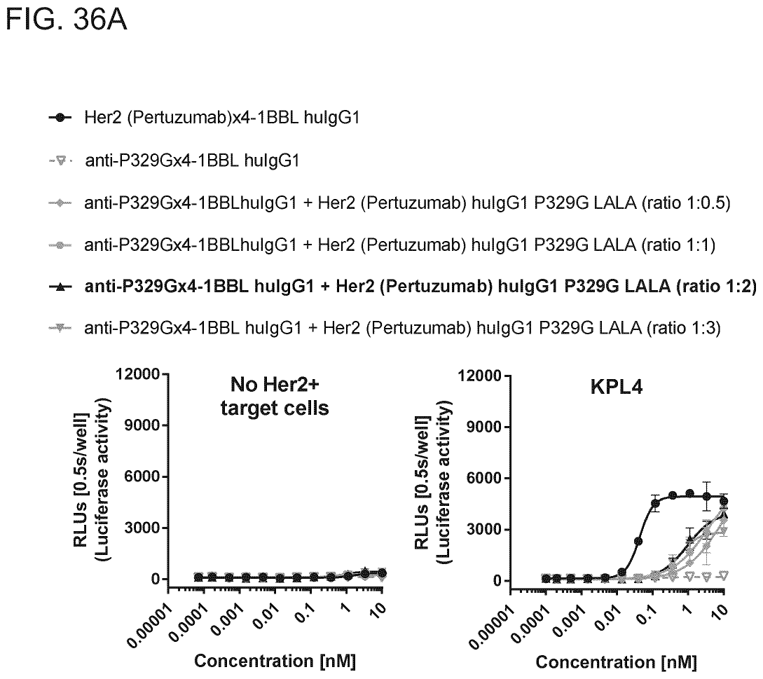

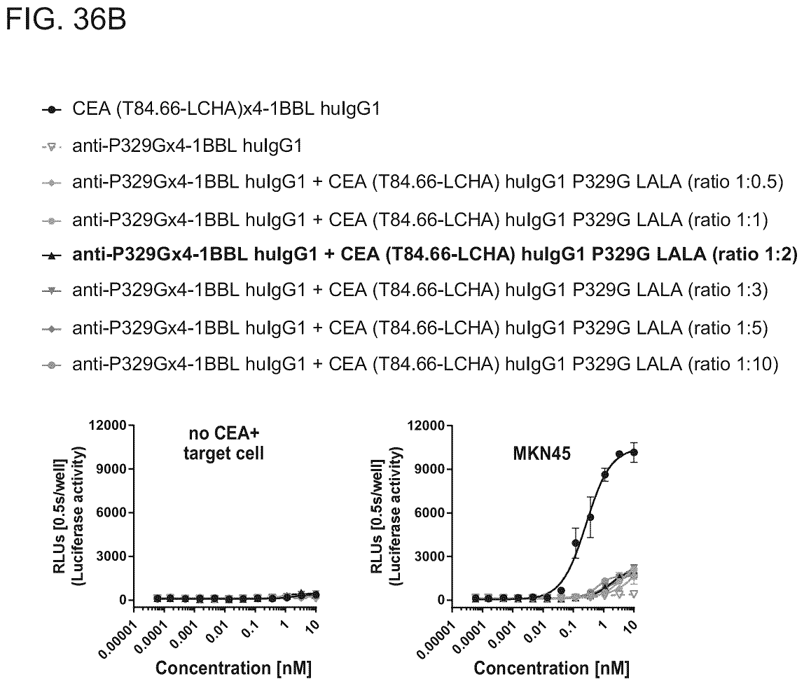

As shown in FIG. 36 different ratios between aP329G-4-1BBL (fixed concentration) and tumor-target specific human IgG1 P329G LALA (variable concentration) were tested to evaluate the optimal ratio. This evaluation shows, that different ratios did not change the activity of aP329G-4-1BBL. The application of an indirect crosslinking (huIgG1 P329G LALA+aP329G-4-1BBL) leads to higher EC50 values if compared with direct-targeted TT-4-1BBL molecules. Intestingly the maximum value of activation was similar of aP329G-4-1BBL if compared to directly targeted Her2 (pertuzumab)-4-1BBL but not, if compared to CEA(T84.66-LCHA)-4-1BBL.

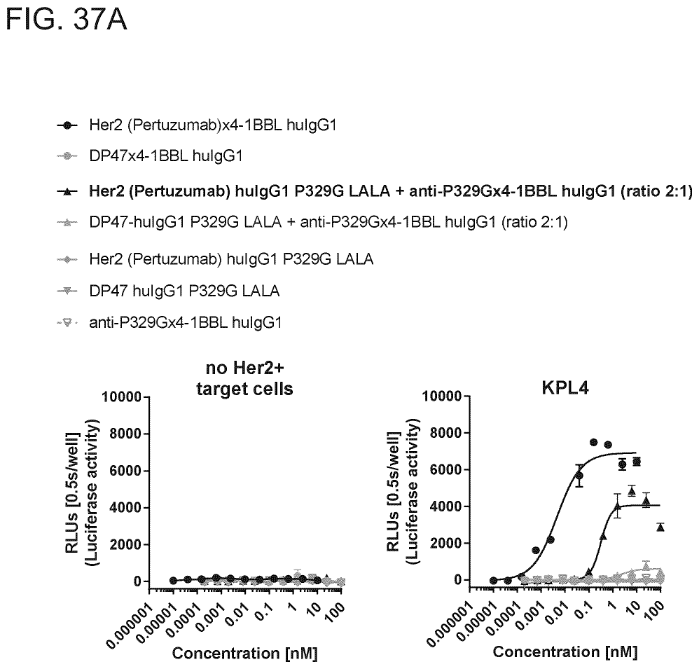

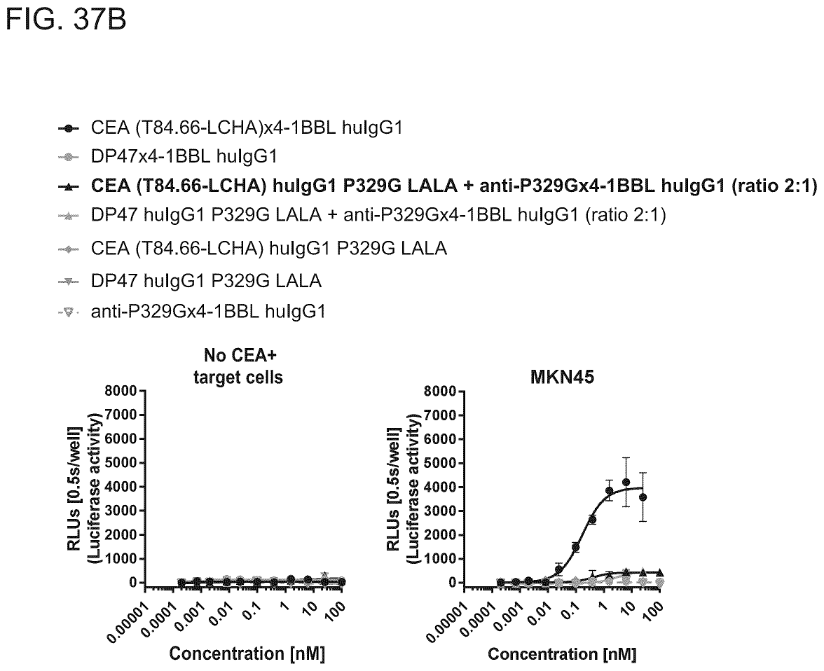

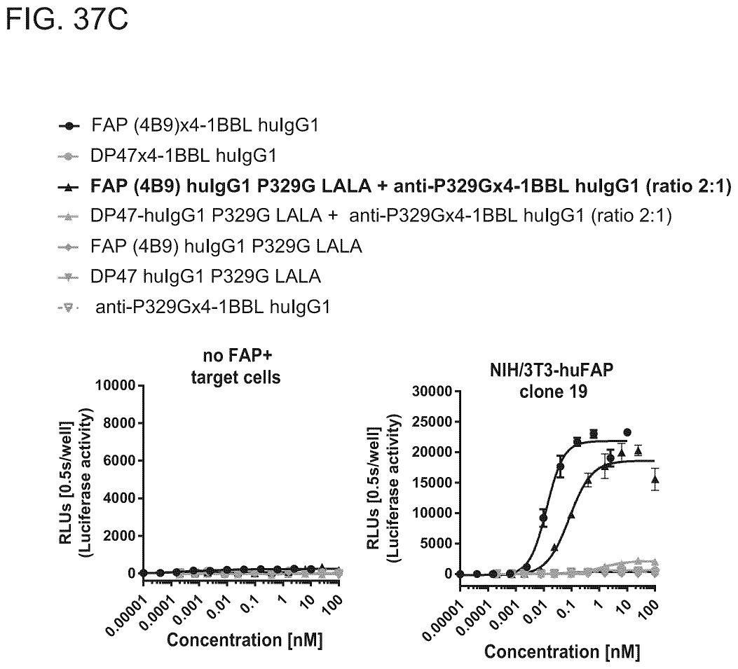

As shown in FIG. 37 aP329G-4-1BBL is functional independent of the used tumor-target specific human IgG1 P329G LALA. Further this functionality is target-specific and concentration dependent. The fitting EC50 values are listed in Table 28. Again EC50 values were reduced of indirect targeted aP329G-4-1BBL if compared to direct tumor-targeted (TT)-4-1BBL Maximum values of activity however were similar if molecules were targeted via Her2 or FAP, whereby targeting via CEACAM5 led to a reduction of the maximum values if 4-1BB was activated via indirectly crosslinked aP329G-4-1BBL and not by TT-4-1BBL. Therefore the differences between aP329G-4-1BBL and TT-4-1BBL is target dependent.

TABLE 28
EC50 values [nM] and maximum activity value [URLs]

| | | Her2 (Pertuzumab) | |
|---|---|---|---|
| | | huIgG1 P329G | |
| | Her2 | LALA + | |
| | (Pertuzumab)- | anti-P329G-4-1BBL | anti-P329G- |
| Her2 + KPL4 | 4-1BBL | (ratio 2:1) | 4-1BBL |
| EC50 [nM] | 0.0004 | 0.317 | n.d. |
| Maximum | 6375 | 4065 | 182 |
| values [URLs] | | | |
| | | CEA (T84.66-LCHA) | |
| | | huIgG1 P329G | |
| | CEA (T84.66- | LALA + | |
| CEACAM5 + | LCHA)- | anti-P329G-4-1BBL | anti-P329G- |
| MKN45 | 4-1BBL | (ratio 2:1) | 4-1BBL |
| EC50 [nM] | 0.34 | 0.41 | n.d. |
| Maximum | 3976 | 4265 | 16 |
| values [URLs] | | | |

| | | FAP (4B9) huIgG1 | |
|---|---|---|---|
| | | P329G LALA + | |
| NIH/3T3-huFAP | FAP (4B9)- | anti-P329G-4-1BBL | anti-P329G- |
| clone 19 | 4-1BBL | (ratio 2:1) | 4-1BBL |
| EC50 [nM] | 0.0072 | 0.083 | n.d. |
| Maximum | 21829 | 18591 | 330 |
| values [URLs] | | | |

### Example 7

### ADCC Competent Molecules

Exemplary Configurations of the Immune Effector Cell Activating Fc Binding Molecule of the Invention

FIG. 38 shows an exemplary illustration of an IgG1 effector molecule able to bind to the P329G mutation (anti-P329G (VH3VL1) huIgG1) of a tumor targeting molecule (e.g. IgG1, SM). (B) Exemplary configuration of the binding mode of the anti-P329G IgG1 effector molecule to the P329G mutation of a tumor targeting IgG and the FcγRIII on immune effector cells. Crosslinking of tumor cell and target cells is induced by the binding of the anti-P329G huIgG1 to the respective tumor targeting IgG. In more detail both Fab arms of the anti-P329G IgG1 can bind the P329G mutation present e.g in an Fc region of an antigen specific IgG. This antigen specific IgG is able to bind the target antigen with two Fab arms. The Fc part of the anti-P329G IgG Fc binding molecule is able to bind to the FcγRIII receptor present on immune effector cells. The formation of the anti-P329Ghu IgG1+antigen targeting IgG complex leads to crosslinking of immune effector cell and tumor cells, resulting in immune effector cell activation and target cell lysis.

ADCC Induction by IgG1 with Glycoengineered Fc (IgG1_{ADCC driver} Antibody) in the Presence of Tumor Antigen Targeting IgG1 with P329G IALA Mutation (IgG1_{TA})

The ability to induce ADCC by the complex of anti-P329G IgG1_{ADCC driver} antibody and anti-CD20 IgG1_{TA} was assessed by quantification of the lactate dehydrogenase (LDH) release of target cells. Therefore PBMC effector cells were isolated from buffy coats, obtained from Zurich blood donation center, in accordance with the Declaration of Helsinki. The buffy coat was diluted 2:1 with PBS and human PBMCs were isolated by density gradient centrifugation (450×g, 30 min at room temperature without break) over Histopaque-1077 (Sigma-Aldrich #10771). PBMCs were harvested from the interphase, washed three times with PBS for 10 min at 350 g. After washing PBMCs were counted using Vi-cell. Cells were prepared at 12.5 Mio/ml in RPM11640+2% FBS+1% Glutamax+1% Gibco™ Antibiotic-Antimycotic (100×) (Catalog number: 15240062) (Assay medium). 0.625 Mio viable cells/well were seeded in 50 μl/well into a transparent, 96-U well plate (TPP). As target cells z-138 (CD20⁺) cells were, counted via Vi-cell and 2500 viable cells/ml were seeded in 50 ul of Assay medium into a co-culture with the PVMCs in the respective wells. The Effector to target ratio was 25:1.

Serial dilution of the different antibodies were prepared in 1:10 dilution steps in assay medium, starting with concentrations of 450 nM of the IgG1_{ADCC driver} antibodies anti-P329G huIgG1 or anti-CD20 (GA101) huIgG1 GE variant and 900 nM of the IgG1_{TA} anti-CD20 (GA101) huIgG1 P329G LALA antigen targeting antibody. The final ratio of IgG1_{ADCC driver} to IgG_{TA} was 1:2. For flow cytometry analysis 380 μl anti-CD107a-PE+3420 μl Assay medium were prepared and 10 μl/well of diluted human anti-CD107a (biologend) was added to the cells. The final volume of the wells was 210 μl. Plate was incubated for 5 h at 37° C. at 5% CO₂ in a humidified incubator.

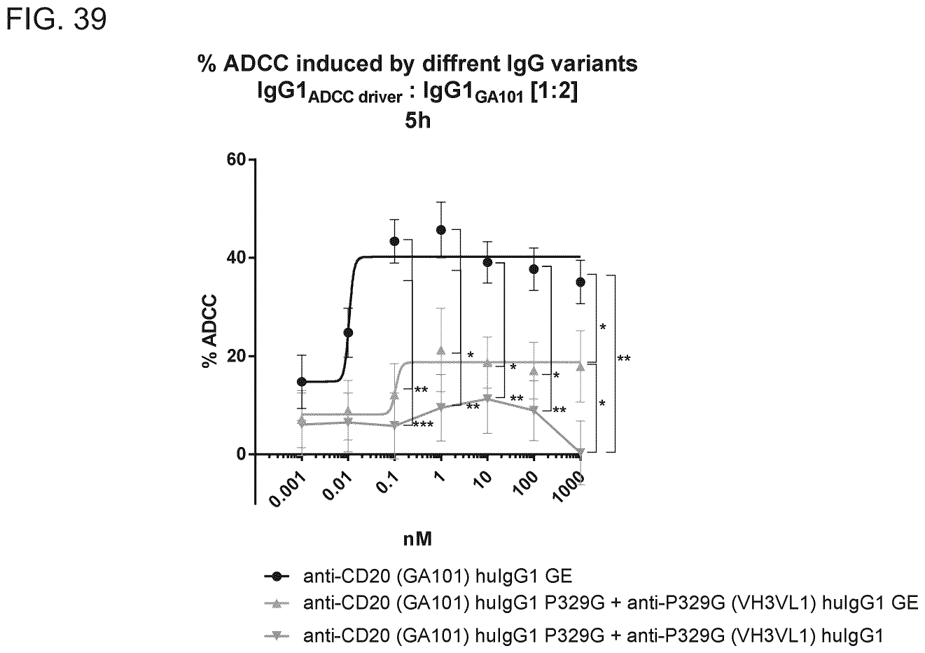

ADCC was assessed after 5 h by quantification of LDH released into cell supernatants by apoptotic/necrotic cells (LDH detection kit, Roche Applied Science, #11 644 793 001). Maximal lysis of the target cells (=100%) was achieved by incubation (for at least 1 h at 37° C. and 5% CO₂ in a humidified incubator) of target cells with a final concentration of 1% Triton X-100 per control well. Minimal lysis (=0%) refers to target cells co-incubated with effector cells without antibody. Using a multichannel pipette, 50 μl of supernatant was transferred to a transparent 96 well plate and 1:1 freshly prepared cytotoxicity reagent according to the manufacturer's protocol and absorbance was measured with Tecan Spark reader over an interval of 10 min. In FIG. 39 technical average values from triplicates and error bars indicate SD are depicted. As p value the New England Journal of Medicine style was used as listed in GraphPadPrism 7. Meaning *=P≤0,033; **=P≤0,002; ***=P≤0.001.

In FIG. 39 it can be observed that anti-CD20 (GA101) huIgG1 GE variant induces a dose-dependent ADCC. The anti-CD20 (GA101) huIgG1 P329G LALA+anti-P329G (VH3VL1) huIgG1 complex that uses the GE variant also mediates dose-dependent ADCC. The anti-CD20 (GA101) huIgG1 P329G LALA+anti-P329G (VH3VL1) huIgG1 wildtyp complex induces ADCC to a lower extend with significant difference compared to the GE variant at the highest concentration of 450 nM of ADCC driving antibody.

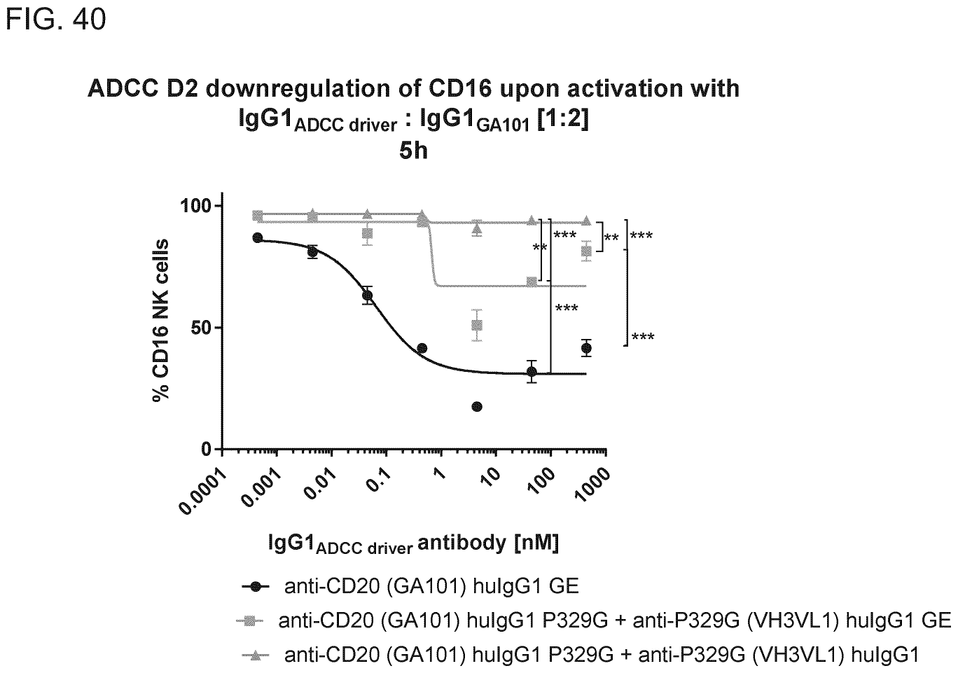

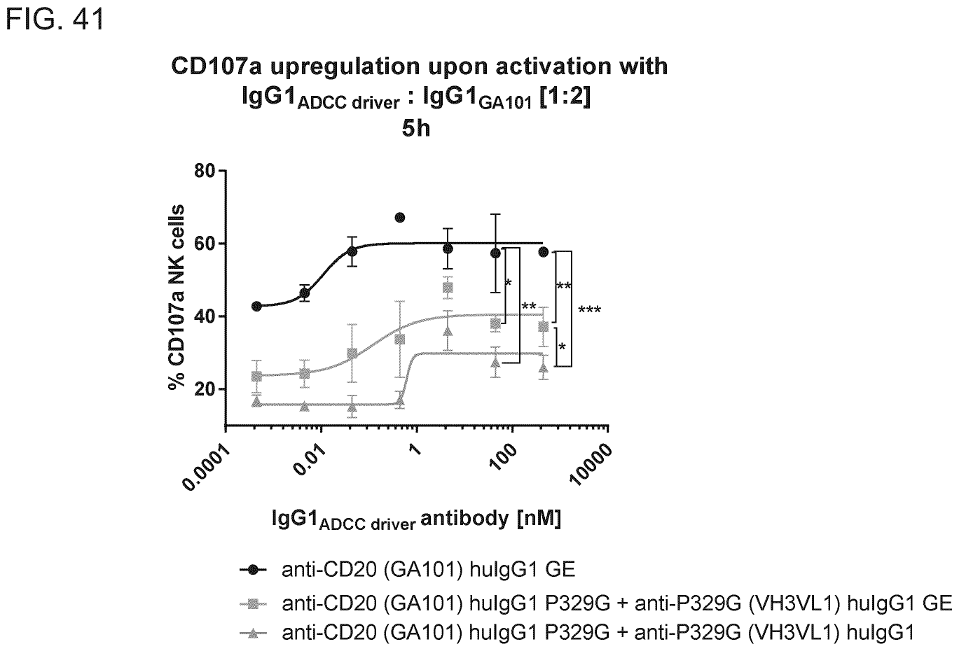

Receptor Regulation on NK Cells Upon Coincubation with IgG1 Possessing a Glycoengineered Fc (IgG1_{ADCC driver} Antibody) The experiments illustrated in FIG. 40 and FIG. 41: build on the experiment described in

FIG. 39. In the experiments illustrated in FIG. 39 only the supernatant of the assay was analysed. Instead, in the experiments illustrated below (FIG. 40 and FIG. 41) the natural killer cells (NK) were characterised with flow cytometry by their downregulation of CD16 (FIG. 40) and upregulation of CD107a (FIG. 33). Therefore the assay plate was centrifuged and supernatant was discarded. Plate was washed three times with PBS. The antibody mix for cell staining was prepared as followed 400 μl CD3-PE/Cy7+400 μl CD56-APC+400 μl CD16-FITC+18800 μl PBS buffer (all antibodies were purchase from biolegend). Cells were stained for 30 min at approximately 4-8° C. in the dark. Plates was then washed two times with FACS buffer (PBS+2% FCS+5 mM EDTA+0.25% sodium acide and the samples were measured by FACSCantoII and analysed using FlowJo Software. In FIG. 40 and FIG. 41 technical average values from triplicates and error bars indicate SD are depicted. As p value the New England Journal of Medicine style was used as listed in GraphPadPrism 7. Meaning *=P≤0,033; **=P≤0,002; ***=P≤0,001. FIG. 40 shows a dose-dependent downregulation of CD16 upon activation with the anti-CD20 (GA101) huIgG1 P329G LALA+anti-P329G (VH3VL1) huIgG1 complex that uses the GE variant and the anti-CD20 (GA101) huIgG1 GE. The use of anti-CD20 (GA101) huIgG1 P329G LALA+anti-P329G (VH3VL1) huIgG1 wildtyp complex does not show a downregulation of the CD16 receptor. In FIG. 41 a dose-dependent upregulation of CD107a on NK cells can be observed when the anti-CD20 (GA101) huIgG1 P329G LALA+anti-P329G (VH3VL1) huIgG1 complex that uses the GE variant or the anti-CD20 (GA101) huIgG1 GE was used. For high concentrations of the antibodies also the use of anti-CD20 (GA101) huIgG1 P329G LALA+anti-P329G (VH3VL1) huIgG1 complex shows an upregulation of CD107a receptor on NK cells. The difference between anti-CD20 IgG1 P329G LALA+anti-P329G IgG1 GE complex and anti-CD20 (GA101) huIgG1 P329G LALA+anti-P329G (VH3VL1) huIgG1 complex is significant for concentrations higher than 45 nM.

Tumor Stroma Targeting huIgG1 (aFAP Clone 4B9) and Anti-P329G Human IgG1 mAb to Induce FcRγIIIa

The capacity of tumor stroma targeting huIgG1 (aFAP clone 4B9) and anti-P329G human IgG1 mAb to induce FcRγIIIa cross-linking and subsequently NFAT activation and ADCC, was assessed using co-cultures of target protein coated beads (human FAP) and Jurkat-NFAT reporter cells (a FcγRIIIA-expressing human acute lymphatic leukemia reporter cell line with a NFAT promoter, Jurkat FcγRIIIa V158_NFAT-RE_luc, Promega, Cat #G9791). Upon simultaneous binding of the anti-P329G huma IgG1 antibody to the P329G mutation of the immobilized huIgG1 (bound to the FAP) and the FcRγIIIa (expressed on Jurkat-NFAT reporter cells), the NFAT promoter is activated and leads to expression of active firefly luciferase. The intensity of luminescence signal (obtained upon addition of luciferase substrate) is proportional to the intensity of CD16 activation and signaling.

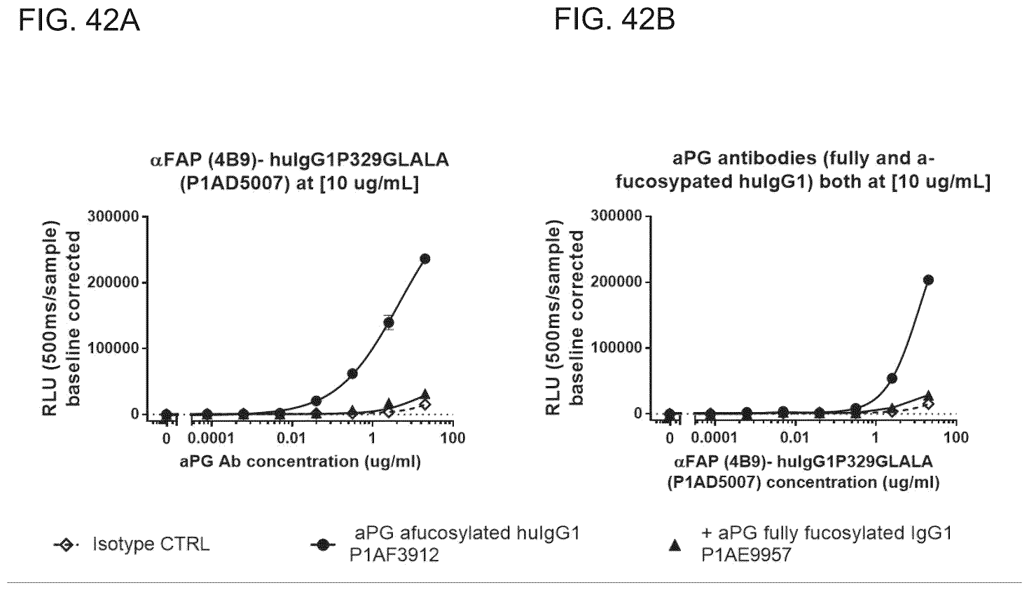

Two conditions were tested. As tumor targeting antibody, either a fixed concentration (10 μg/mL) of anti-FAP (4B9) P329G LALA huIgG1 was used in combination with an 8-fold decreasing dilution series of the anti-P329G huIgG1 ranging from the highest final concentration 20 μg/mL to 7.6 *10-5 μg/mL (FIG. 42A). Or, as tumor targeting antibody, an 8 fold decreasing serial titration of anti-FAP (4B9) P329G LALA huIgG1 ranging from the highest final concentration 20 μg/mL to 7.6 *10-5 μg/mL was used in combination with a fixed concentration (10 μg/mL) of the anti-P329G huIgG1 (FIG. 42B). It is well known that the effector function of ADCC competent antibodies is modulated by the N-linked glycosylation in the Fc region of the antibody. In particular, absence of core fucose on the Fc N-glycan has been shown to increase IgG1 Fc binding affinity to the FcγRIIIa present on immune effector cells such as natural killer cells and lead to enhanced ADCC activity. Therefore, the anti-P329G huIgG1 was tested as fully fucosylated and afucosylated human IgG1 isotype.

For the assay, Jurkat FcγRIIIa V158_NFAT-RE_luc reporter cells were harvested. Cells were counted and viability was assessed using Cedex HiRes cell counter. The needed amount was harvested by centrifugation for 5 min at 300 g. About 2×10⁴ cells/well were plated in AIM-V assay medium in a white flat-bottom 384-well-plate (Corning #3826). Subsequently, FAP coated beads were added at a count of 5×10¹ beads/well to reach an effector to target ratio of 4 to 1. The different antibody combinations were added as well to the 384 wells into a final volume of 30 μl. As substrate the ONE-Glo Luciferase Assay System (E6120, Promega) was used according to the manufacturer's protocol allowing for an endpoint measurement of relative luminescence units (RLU). Readout was performed after 22 hours using a Tecan Spark 10M luminescence plate reader and 500 ms integration time. Each point in FIG. 42 represents the mean value of technical duplicates of one experiment. Standard error of the mean is indicated by error bars. FAP coated beads were prepared from Dynabeads™ M-280 Streptavidin (Invitrogen, #11205D) coating them with biotinylated human FAP (Roche) as described in the manufacturer's protocol. Briefly, 12×10⁶ PBS washed beads were coated for 30 minutes with 2.43 μg biotinylated human FAP in 21.5 μL PBS under gentle rotation (MACSmix™ Tube Rotator).

Only the combination of anti-FAP (clone 4B9) human IgG1 P329GLALA and anti-P329G human IgG1 mAb induces dose dependent NFAT activation in Jurkat FcγRIIIa V158_NFAT-RE_luc reporter cells, which is a measure of ADCC competency. Neither anti-FAP (clone 4B9) human IgG1 P329GLALA nor anti-P329G human IgG1 mAb alone was able to do so (first data point of dose response curves). Thus, the optimal concentrations for target saturation and ADCC competency can be chosen independently to optimize treatment efficacy in patients. The afucosylated version of anti-P329G human IgG1 mAb showed superior ADCC potency compare to the same clone as fully fucosylated human IgG1 mAb.

### Example 8

Jurkat NFAT Luc Kinetic T Cell Activation Assay

Kinetics of T cell activation induced by the anti-P329G (VH3VL1)×CD3 (P035.093) 2+1 TCB, LALA Fc was measured using Jurkat NFAT reporter cells in presence of HeLa tumor cells, over a period of 24 h with 2 h intervals.

Jurkat NFAT reporter cells (GloResponse Jurkat NFAT-RE-luc2P, Promega #CS176501) are a CD3-expressing human acute lymphatic leukemia reporter cell line with an NFAT-response element controlling the expression of firefly luciferase. Upon crosslinking of the CD3, the NFAT promoter is activated, leading to a dose-dependent expression of the luciferase. Adding a luciferase substrate results in a luminescent signal, reflecting the strength of the Jurkat NFAT T cell activation. The crosslinking can be initiated by a TCB, which simultaneously binds tumor targets and the CD3 on the Jurkat cells.

The Jurkat NFAT activation induced by the anti-P329G (VH3VL1)×CD3 (P035.093) 2+1 TCB was tested in presence of tumor-targeting P329G LALA huIgG1s.

The capacity of tumor targeting anti-FOLR1 P329G LALA huIgG1 with anti-P329G (VH3VL1)×CD3 (P035.093) 2+1 TCB, P329R LALA Fc to induce T cell activation was assessed using co-cultures of tumor antigen positive target cells (HeLa) and Jurkat-NFAT reporter cells.

As a tumor targeting antibody, anti-FolR1 (16D5) P329G LALA huIgG1 was used in combination with the anti-P329G (VH3VL1)×CD3 (P035.093) 2+1 TCB, P329R LALA Fc, in molar ratio IgG:TCB 2:1. As a positive control, a direct tumor-targeting anti-FoIRI (16D5)×CD3 (P035.093) 2+1 TCB was used.

A non-binding DP47×CD3 (P035.093) 2+1 TCB was used as a negative control. Three TCB concentrations were tested: 0 nM, 0.05 nM, 5 nM, to assess the dose dependency of the Jurkat-NFAT-reporter cell activation.

As a preparation for the assay, HeLa human tumor cells were harvested. Growth medium was removed from the cell culture flask and cells were washed once with phosphate-buffered saline (PBS, Gibco #20012). After removing PBS, cells were trypsinised (Trypsin-EDTA (0.05%), phenol red, Gibco #25300-054). Cell count and viability was determined using a Countess Automated Cell Counter (Invitrogen #C10227). 0.002×10⁶ cells/well (10 μl/well) were plated in flat bottom, white 384-well plates (Corning #353988) in assay medium (RPMI 1640, 10% FBS, 1% GlutaMAX), one day before the assay. On the assay day, Jurkat-NFAT reporter cells were harvested. The cells were counted and assessed for viability using the Countess device. The necessary amount was harvested by centrifugation for 5 min at 350 g. 0.01×106 cells/well (10 μl/well) were plated in assay medium to obtain a final effector-to-target cell ratio (E:T) of 5:1. Subsequently, antibody dilutions were prepared in the assay medium, and were added to the 384 well plate to obtain a final volume of 40 μl. As a luciferase substrate, GloSensor™ cAMP Assay (Promega #E1290) was used according to the manufactures protocol, allowing for a kinetic measurement of relative luminescence units (RLU). Readout was performed automatically every 2 h using a Tecan Spark 10M reader with temperature and CO₂ control (37° C. and 5% CO₂), and humidified atmosphere, thus allowing the automatized measurement without disturbing the culture conditions. The luminescent signal was acquired for 300 ms/well, and calculated to reflect RLU/s per well.

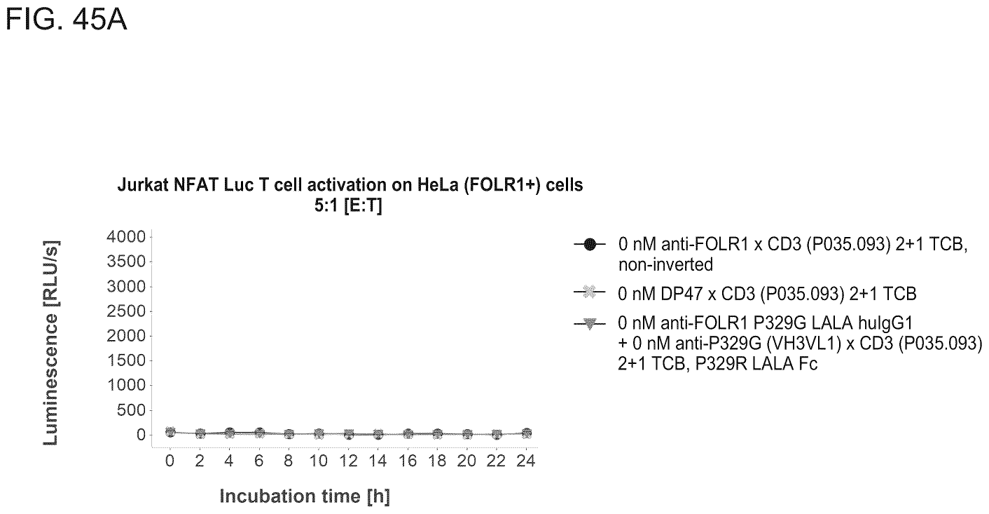

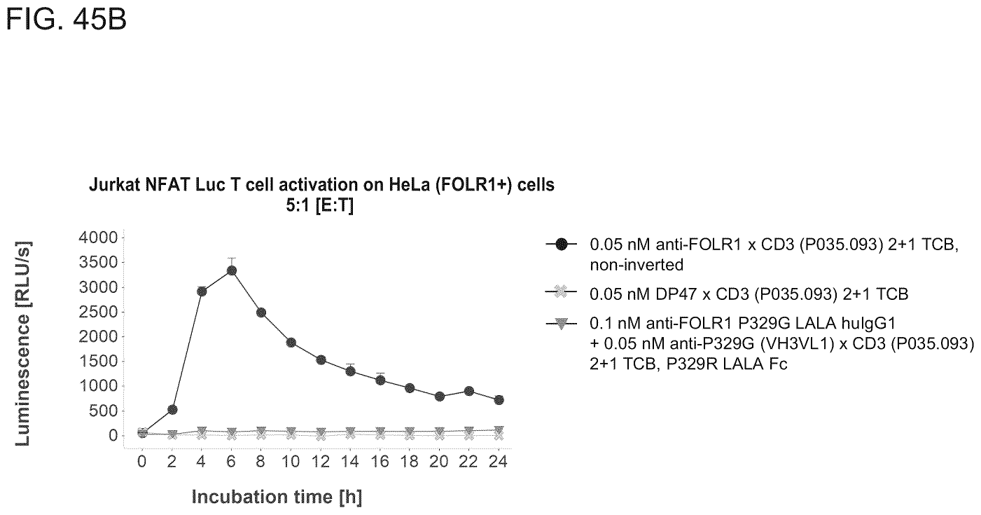

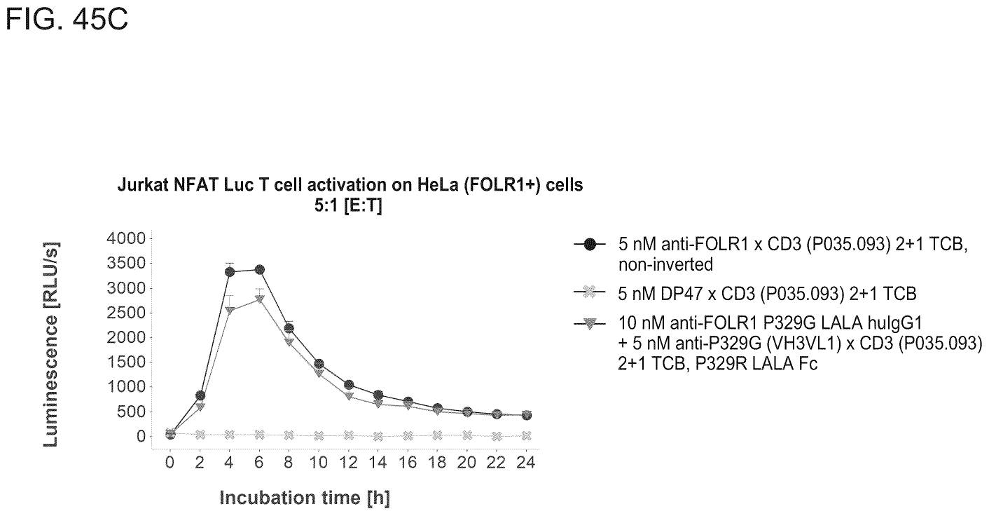

FIG. 45 represents experiments performed with 0 nM (FIG. 45A), 0.05 nM (FIG. 45B) and 5 nM (FIG. 45C) concentration of the TCBs. The data points show mean values of the technical triplicates of one experiment. Error bars indicate standard deviation. The kinetic activity of the anti-P329G (VH3VL1)×CD3 (P035.093) 2+1 TCB, P329R LALA Fc was assessed, and the optimal incubation for the Jurkat-NFAT-Luc reporter assays was determined to be between 4 h and 6 h. The anti-P329G TCB concentration needed to reach the effect comparable to the direct TCB was determined to be around 5 nM.

Jurkat NFAT Luc Activation Assay on a Set of FOLR⁺ Cells: HeLa, JAR, OVCAR-3, SKOV-3

T cell activation capacity of the FOLR1-targeting P329G LALA huIgG1 with anti-P329G (VH3VL1)×CD3 (P035.093) 2+1 TCB, LALA Fc across several FOLR1⁺ tumor cell lines was assessed with Jurkat-NFAT reporter cells (GloResponse Jurkat NFAT-RE-luc2P, Promega #CS176501) described above.

The Jurkat NFAT activation, induced by the anti-P329G (VH3VL1)×CD3 (P035.093) 2+1 TCB, LALA Fc, was tested in presence of tumor-targeting P329G LALA huIgG1s.

As a tumor targeting antibody, anti-FolR1 (16D5) P329G LALA huIgG1 was used in combination with the anti-P329G (VH3VL1)×CD3 (P035.093) 2+1 TCB, LALA Fc, in molar ratio IgG:TCB 2:1. A non-binding DP47 P329G LALA huIgG1 combined with the anti-P329G TCB, in molar ratio IgG:TCB 2:1, was used as a negative control. As a positive control, a direct tumor-targeting anti-FolR1 (16D5)×CD3 (P035.093) 2+1 TCB was used.

The anti-P329G TCBs were mixed with the respective IgGs at the highest concentration in the molar ratio IgG:TCB 2:1, and were titrated together. All TCBs were serially diluted 4-fold starting from 75 nM of the TCBs, resulting in a concentration range from 75 nM to 1.12×10⁻⁶ nM.

The experiment was performed as described before, with a differing readout protocol, consisting of a single timepoint. HeLa, JAR, OVCAR-3 and SKOV-3 were used as target cells. The assay components were incubated for 6 h in 37° C. and 5% CO₂. After the incubation time, a luciferase substrate, ONE-Glo™ Luciferase Assay reagent (Promega #E6120) was used according to the manufactures protocol, allowing for a measurement of relative luminescence units (RLU). Readout was performed using a Tecan Spark 10M reader. The luminescent signal was acquired for 300 ms/well, and calculated to reflect RLU/s per well.

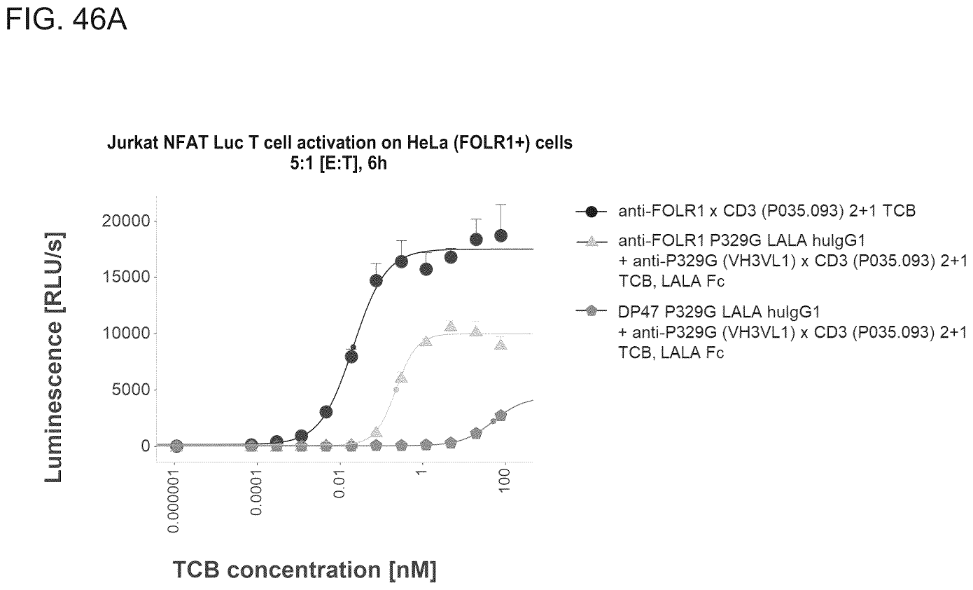

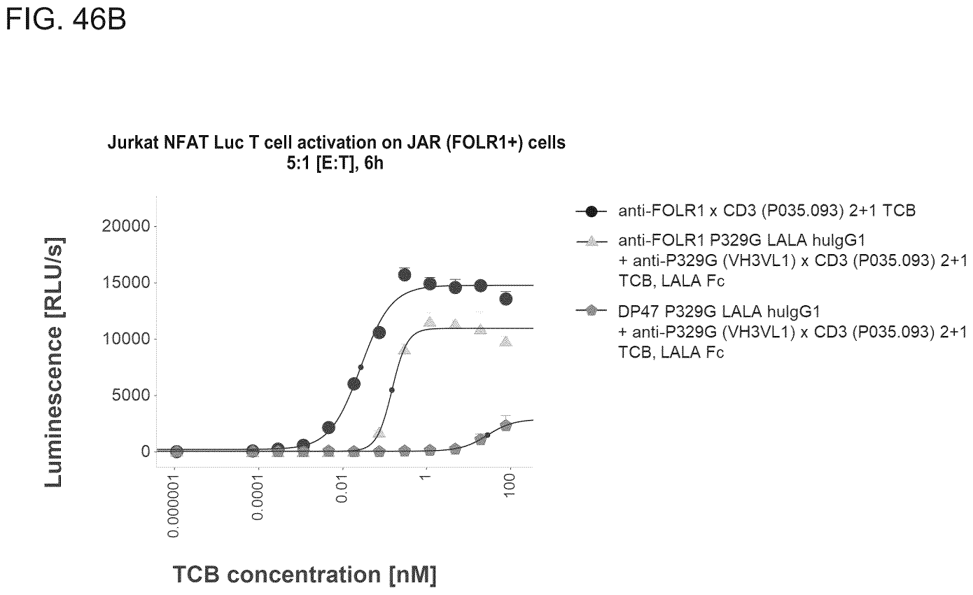

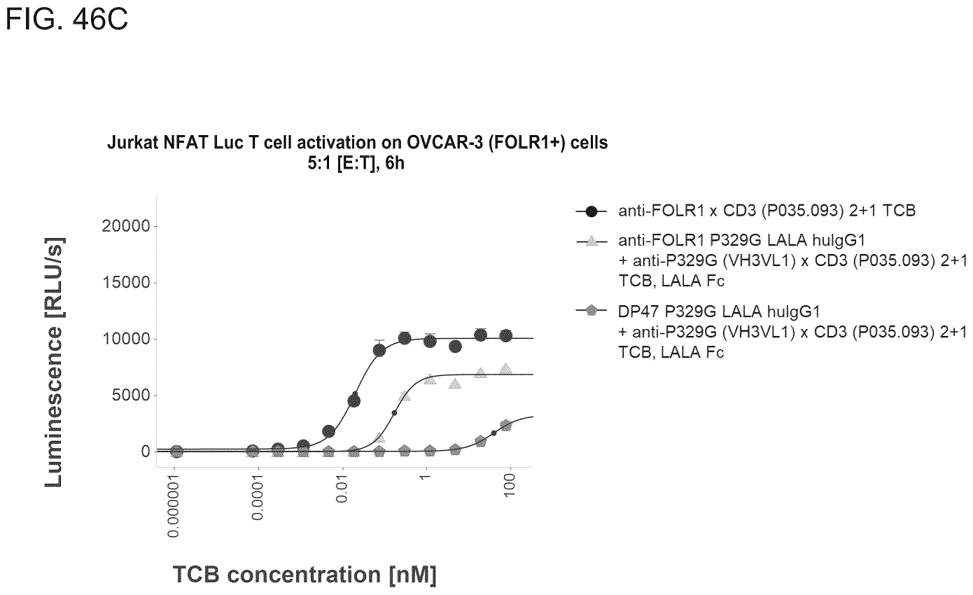

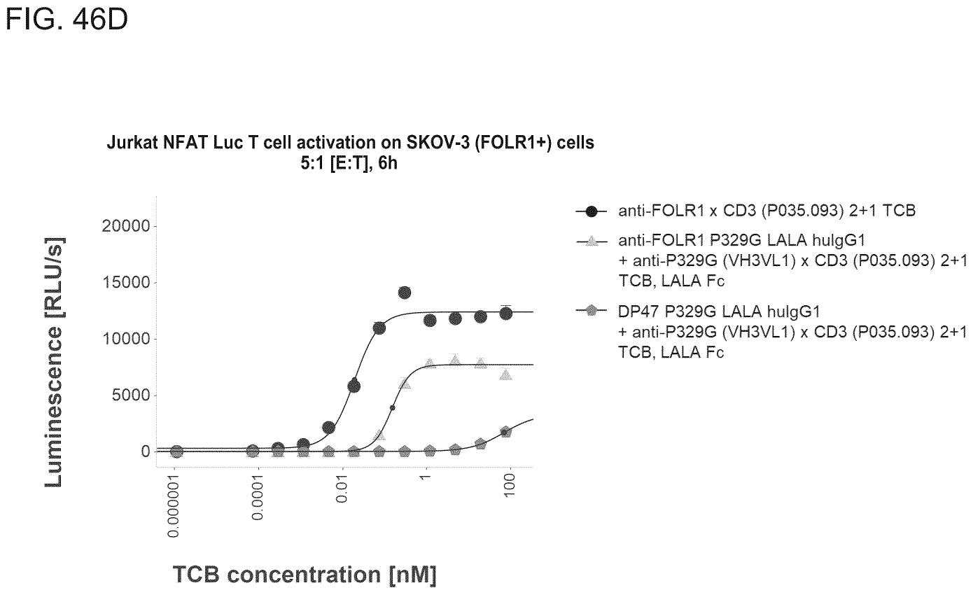

FIG. 46 depicts results from experiments performed with HeLa (FIG. 46A), JAR (FIG. 46B), OVCAR-3 (FIG. 46C), SKOV-3 (FIG. 46D). The data points show mean values of the technical triplicates of one experiment. Error bars indicate standard deviation.

The anti-P329G (VH3VL1)×CD3 (P035.093) 2+1 TCB, LALA Fc show a dose-dependent T cell activation capacity in presence of anti-FOLR1 P329G LALA huIgG1 across all tested FOLR1+ cell lines, validating the IgG1 adaptor as a viable strategy. In presence of DP47 P329G LALA huIgG1, the anti-P329G TCB is ineffective at most of the tested concentrations, showing residual activity at 75 nM.

Jurkat NFAT Activation Assay on HeLa (FOLR1+) Cells; Comparison of P329R LALA Fc and LALA Fc Versions of the Anti-P329G TCB

T cell activation capacity of the anti-P329G (VH3VL1)×CD3 (P035.093) 2+1 TCB, LALA Fc and the anti-P329G (VH3VL1)×CD3 (P035.093) 2+1 TCB, P329R LALA Fc was assessed with Jurkat-NFAT reporter cells (GloResponse Jurkat NFAT-RE-luc2P, Promega #CS176501) described above.

The Jurkat NFAT activation induced by the anti-P329G (VH3VL1)×CD3 (P035.093) 2+1 TCB with LALA Fc or P329R LALA Fc was tested in presence of a tumor-targeting P329G LALA huIgG1s.

As a tumor targeting antibody, anti-FolR1 (16D5) P329G LALA huIgG1 was used in combination with the anti-P329G (VH3VL1)×CD3 (P035.093) 2+1 TCB, P329R LALA Fc or anti-P329G (VH3VL1)×CD3 (P035.093) 2+1 TCB, LALA Fc in a molar ratio IgG:TCB 2:1. Anti-P329G TCB without an P329G LALA huIgG1 was used as a negative control. As a positive control, a direct tumor-targeting anti-FolR1 (16D5)×CD3 (P035.093) 2+1 TCB was used.

The anti-P329G TCBs were mixed with the respective IgGs at the highest concentration in the molar ratio IgG:TCB 2:1, and were titrated together. All TCBs were serially diluted 10-fold starting from 75 nM of the TCBs, resulting in a concentration range from 75 nM to 75×10⁻⁹ nM.

The experiment was performed as described before, with a single timepoint measurement. HeLa were used as target cells. The assay components were incubated for 6 h in 37° C. and 5% CO₂ before addition of the luciferase substrate and the readout procedure.

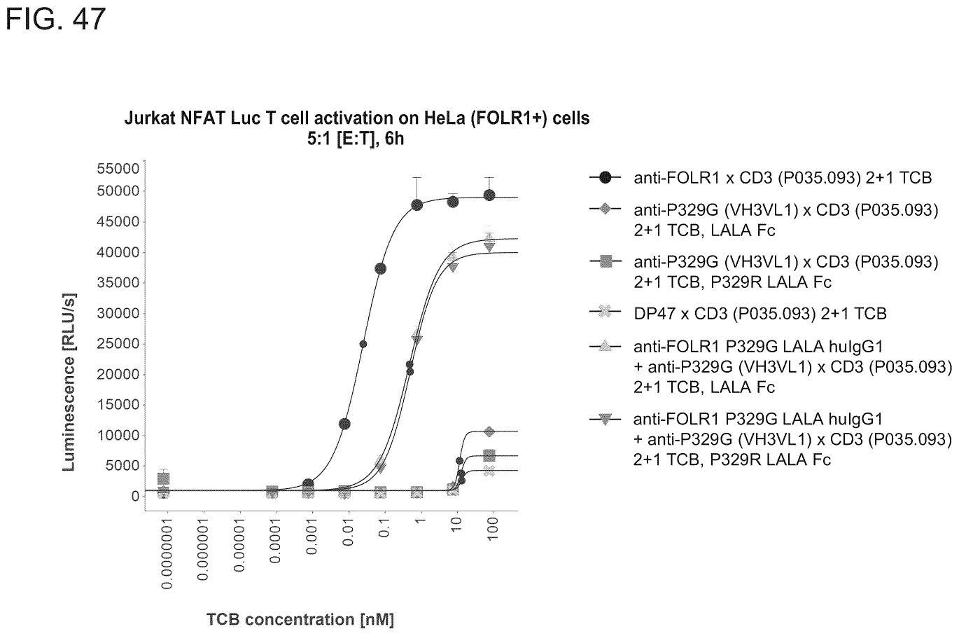

FIG. 47 depicts results from experiments performed with HeLa cells. The data points show mean values of the technical triplicates of one experiment. Error bars indicate standard deviation. Both versions of the a-P329G TCB, the anti-P329G (VH3VL1)×CD3 (P035.093) 2+1 TCB, LALA Fc and the anti-P329G (VH3VL1)×CD3 (P035.093) 2+1 TCB, P329R LALA Fc show a dose-dependent T cell activation capacity in presence of anti-FOLR1 P329G LALA huIgG1. Without the P329G LALA huIgG1, both anti-P329G TCBs are ineffective at most of the tested concentrations, showing residual activity at 75 nM. The anti-P329G (VH3VL1)×CD3 (P035.093) 2+1 TCB, P329R LALA Fc shows less non-specific activation.

### Example 9

Primary Human T Cell Activation Assay on SKOV-3 (FOLR1+) Cells; Comparison of P329R LALA Fc and LALA Fc Versions of the Anti-P329G TCB; Comparison of T Cells and PBMCs as Effector Cells

T cell activation capacity of the anti-P329G (VH3VL1)×CD3 (P035.093) 2+1 TCB, LALA Fc and the anti-P329G (VH3VL1)×CD3 (P035.093) 2+1 TCB, P329R LALA Fc was assessed with primary human pan T cells or PBMCs cells from a healthy donor.

T cell activation induced by the anti-P329G (VH3VL1)×CD3 (P035.093) 2+1 TCB with LALA Fc or P329R LALA Fc was tested in presence of a tumor-targeting P329G LALA huIgG1.

As a tumor targeting antibody, anti-FolR1 (16D5) P329G LALA huIgG1 was used in combination with the anti-P329G (VH3VL1)×CD3 (P035.093) 2+1 TCB, P329R LALA Fc or anti-P329G (VH3VL1)×CD3 (P035.093) 2+1 TCB, LALA Fc in a molar ratio IgG:TCB 2:1. A non-binding DP47 P329G LALA huIgG1 combined with either of the anti-P329G TCBs, in molar ratio IgG:TCB 2:1, was used as a negative control. As a positive control, a direct tumor-targeting anti-FolR1 (16D5)×CD3 (P035.093) 2+1 TCB was used. The target cells used were SKOV-3 (FOLR1+). The anti-P329G TCBs were mixed with the respective IgGs at the highest concentration in the molar ratio IgG:TCB 2:1, and were titrated together. All TCBs were serially diluted 10-fold starting from 50 nM of the TCBs, resulting in a concentration range from 50 nM to 50×10⁻⁸ nM.

As a preparation for the assay, SKOV-3 (FOLR1+) human tumor cells were harvested. Growth medium was removed from the cell culture flask and cells were washed once with phosphate-buffered saline (PBS, Gibco #20012). After removing PBS, cells were trypsinised (Trypsin-EDTA (0.05%), phenol red, Gibco #25300-054). Cell count and viability was determined using a Countess Automated Cell Counter (Invitrogen #C10227). 0.015×10⁶ cells/well (30 μl/well) were plated in flat bottom, white 384-well plates (Corning #353988) in assay medium (RPMI 1640, 10% FBS, 1% GlutaMAX), one day before the assay. On the assay day, frozen PBMCs and pan T cells previously isolated from blood of a healthy donor were thawed. The cells were counted and assessed for viability using the Countess device. The necessary amount was harvested by centrifugation for 5 min at 350 g. 0.75×10⁶ cells/well (10 μl/well) were plated in assay medium to obtain a final effector-to-target cell ratio (E:T) of 5:1. Subsequently, antibody dilutions were prepared in the assay medium, and were added to the 384 well plate to obtain a final volume of 100 μl.

The assay components were incubated for 48 h or 72 h in 37° C. and 5% CO₂. At the 48 h or 72 h timepoint, PBMCs and T cells were harvested and analyzed for CD25 and CD69 expression as markers of T cell activation.

In detail, the supernatant was removed from the plates, 60 μl of PBS was added to each well and cells were transferred to a 96 well U bottom plate for FACS staining. The plates were centrifuged for 3 min at 600×g, supernatant was removed and cells were washed with 200 μl of PBS per well. The plate was again centrifuged for 3 min at 600×g and supernatant was removed. Subsequently, 30 μl of the antibody mix containing Zombie Aqua™ Fixable Viability Kit (Biolegend, #423102), anti-huCD4 PerCP/Cy5.5 (Biolegend, #344608), anti-huCD8a BV711 (Biolegend, #301044), anti-huCD25 PE (Biolegend, #302606) and anti-huCD69 FITC (Biolegend, #310904) was added to each well. The plates were incubated for 30 min at 4° C. Afterwards, the cells were washed twice with FACS buffer and re-suspended in 60 μl of FACS buffer per well. The cells were acquired using a BD FACSymphony A3 flow cytometer.

FIG. 48A to FIG. 48D depicts results from experiments performed with SKOV-3 cells (FIG. 48A, FIG. 48B) and without target cells (FIG. 48C, FIG. 48D). As effector cells, pan T cells (FIG. 48A, FIG. 48C) and PBMCs (FIG. 48B, FIG. 48D) were used, to assess the effect of FcγR-bearing cells on the non-specific activation o anti-P329G TCBs with LALA and P329R LALA Fc. The data points show mean values of the technical triplicates of one experiment. Error bars indicate standard deviation.

Both versions of the a-P329G TCB, the anti-P329G (VH3VL1)×CD3 (P035.093) 2+1 TCB, containing LALA Fc or P329R LALA Fc show a dose-dependent T cell activation capacity in presence of anti-FOLR1 P329G LALA huIgG1. Without the P329G LALA huIgG1, The anti-P329G (VH3VL1)×CD3 (P035.093) 2+1 TCB, P329R LALA Fc is ineffective, showing no non-specificity. The anti-P329G (VH3VL1)×CD3 (P035.093) 2+1 TCB, LALA Fc with PBMCs shows high non-specific T cell activation, both with and without target cells. The anti-P329G (VH3VL1)×CD3 (P035.093) 2+1 TCB, P329R LALA Fc is superior to the LALA Fc version in terms of suppressing non-specific activation.

### Example 10

Jurkat NFAT Activation Assay on a Set of Tumor Cells with Differing Target Proteins; Comparison of TCB Activity with Different Adaptor P329G LALA huIgG Adaptor Molecules

T cell activation capacity of three a-P329G TCBs with three different CD3 binders (P035.093, CH2527, Clone 22) was assessed. The assay was performed with Jurkat-NFAT reporter cells (GloResponse Jurkat NFAT-RE-luc2P, Promega #CS176501) described in Example 8. The tested TCBs were the following: anti-P329G (VH3VL1)×CD3 (P035.093) 2+1 TCB, P329R LALA Fc, anti-P329G (VH3VL1)×CD3 (CH2527) 2+1 TCB, P329R LALA Fc, anti-P329G (VH3VL1)×CD3 (Clone2 22) 2+1 TCB, P329R LALA Fc.

The Jurkat NFAT activation induced by the different anti-P329G TCBs was tested in presence of a tumor-targeting P329G LALA huIgG1s, in a molar ratio IgG:TCB 2:1, on a set of tumor cell lines expressing the tumor targets. A non-binding DP47 P329G LALA huIgG1 combined with the anti-P329G TCBs, in molar ratio IgG:TCB 2:1, were used as negative controls. As a positive control, a direct tumor-targeting 2+1 TCB was used.

The anti-P329G TCBs were mixed with the respective IgGs at the highest concentration in the molar ratio IgG:TCB 2:1, and were titrated together. All TCBs were serially diluted 10-fold starting from 50 nM of the TCBs, resulting in a concentration range from 50 nM to 50×10⁻⁸ nM.

The experiment was performed as described in Example 8, with a single timepoint measurement. The assay components were incubated for 6 h in 37° C. and 5% CO₂ before addition of the luciferase substrate and the readout procedure.

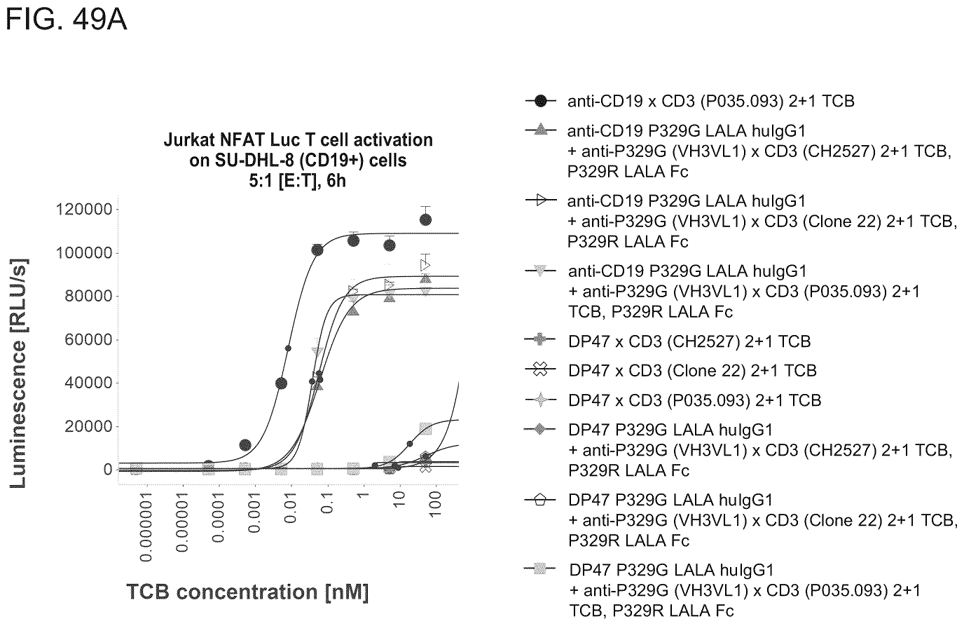

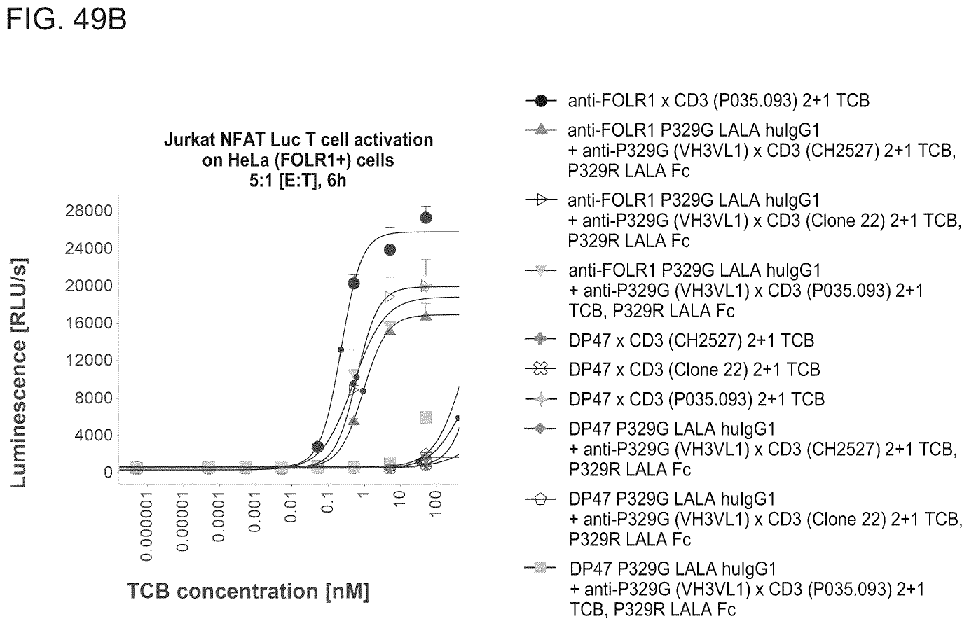

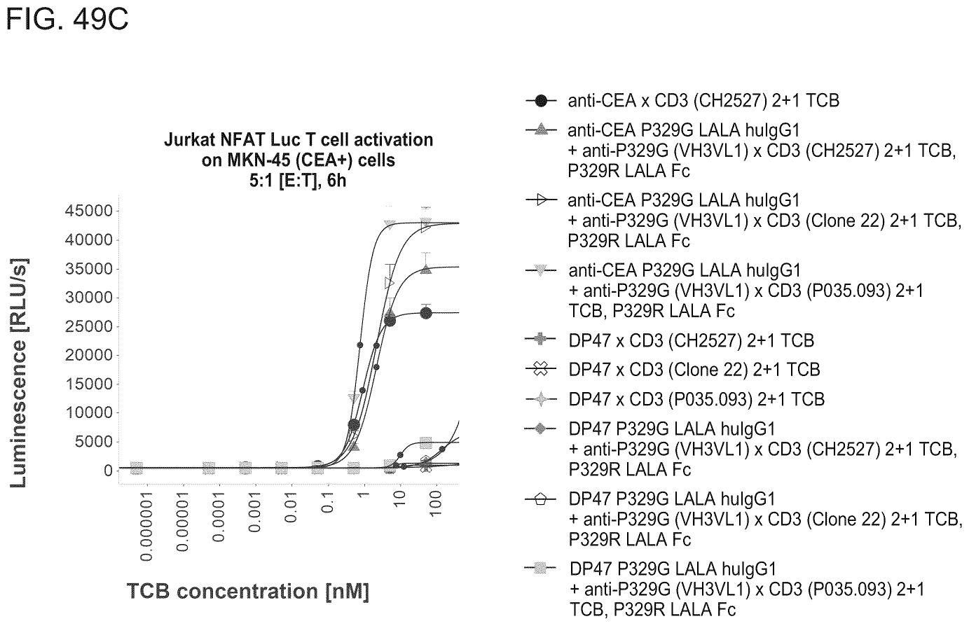

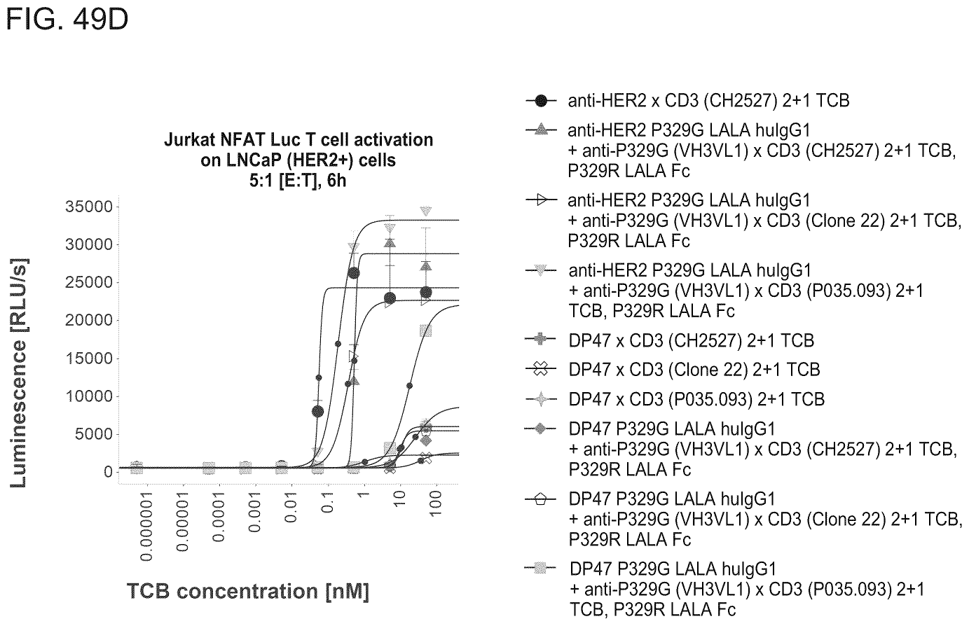

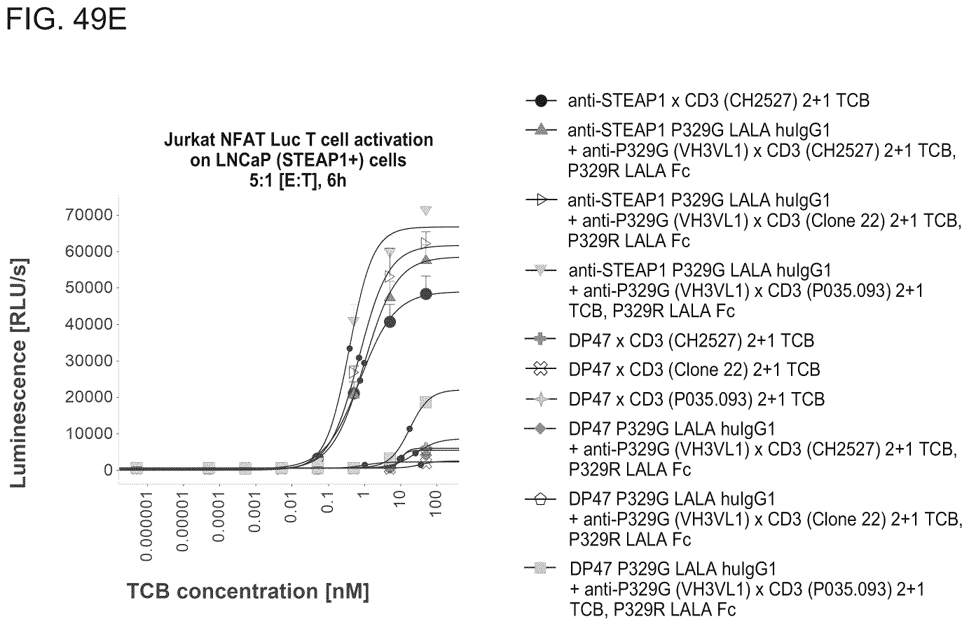

FIG. 49A to FIG. 49E depicts results from the Jurkat NFAT T cell activation experiments with different targeted P329G LALA huIgG1s and target-expressing tumor cell lines. The subfigures show the results with molecules targeting CD19 (FIG. 49A), FOLR1 (FIG. 49B), CEA (FIG. 49C), HER2 (FIG. 49D), STEAP1 (FIG. 49E). The data points show mean values of the technical triplicates of one experiment. Error bars indicate standard deviation. As an example, FIG. 49A shows an assay performed with SU-DHL-8 (CD19+) tumor cells, and anti-CD19 P329G LALA huIgG1 as a tumor-targeting molecule. All three CD3 binder versions of the -P329G TCB, the anti-P329G (VH3VL1)×CD3 2+1 TCB, P329R LALA Fc activate the T cells in a dose-dependent manner. The anti-P329G (VH3VL1)×CD3 (P035.093) 2+1 TCB, P329R LALA Fc induces the most non-specific T cell activation when combined with DP47 P329G LALA huIgG1, as compared to other CD3 binders, rendering it inferior to the other clones.

### Example 11

Tumor Cell Lysis Assay with Primary Human T Cells

The capacity of the anti-P329G (VH3VL1)×CD3 (P035.093) 2+1 TCB, P329R LALA Fe to induce T-cell mediated tumor cell lysis was assessed by an Incucyte killing assay. Additionally, a comparison of three versions of the anti-P329G TCB with three different CD3 binders (P035.3093, CH2527, Clone 22) was performed in this assay. The assay utilizes imaging-based quantification of tumor cells expressing a red fluorescent protein over time inside of an incubator (37° C. and 5% CO₂ humidified atmosphere) with the Incucyte S3 device (Essen Bioscience, #4647). T cell activation induced by the anti-P329G (VH3VL1)×CD3 2+1 TCB, P329R LALA Fc was tested in presence of a tumor-targeting P329G LALA huIgG1s, and primary human pan T cells from a healthy donor. The tested TCBs were: anti-P329G (VH3VL1)×CD3 (P035.093) 2+1 TCB, P329R LALA Fc, anti-P329G (VH3VL1)×CD3 (CH2527) 2+1 TCB, P329R LALA Fc, anti-P329G (VH3VL1)×CD3 (Clone 22) 2+1 TCB, P329R LALA Fc.

As a tumor targeting antibody, anti-FolR1 (16D5) P329G LALA huIgG1 (for HeLa NLR cells) or anti-CEA (T84.66-LCHA) P329G LALA huIgG1 (for MKN-45 NLR cells) was used in combination with the anti-P329G (VH3VL1)×CD3 2+1 TCB, P329R LALA Fc in a molar ratio IgG:TCB 2:1. A non-binding DP47 P329G LALA huIgG1 combined with either of the anti-P329G TCBs, in molar ratio IgG:TCB 2:1, was used as a negative control. As a positive control, a direct tumor-targeting anti-FolR1 (16D5)×CD3 (P035.093) 2+1 TCB or anti-CEA (T84.66-LCHA) x CD3 (CH2527) 2+1 TCB was used.

The anti-P329G TCBs were mixed with the respective huIgG1s at the final concentration in the molar ratio IgG:TCB 2:1, resulting in 1 nM of the IgGs and 0.5 nM of the TCBs.

As a preparation for the assay, HeLa NLR and MKN-45 NLR human tumor cells were harvested. Growth medium was removed from the cell culture flask and cells were washed once with phosphate-buffered saline (PBS, Gibco #20012). After removing PBS, cells were trypsinised (Trypsin-EDTA (0.05%), phenol red, Gibco #25300-054). Cell count and viability was determined using a Countess Automated Cell Counter (Invitrogen #C10227). 0.004×10⁶ cells/well (100 μl/well) were plated in a flat-bottom, clear bottom 96-well-plate (TPP #Z707902) in assay medium (RPMI 1640, 10% FBS, 1% GlutaMAX), one day before the assay. On the assay day, frozen pan T cells previously isolated from blood of a healthy donor were thawed. The cells were counted and assessed for viability using the Countess device. The necessary amount was harvested by centrifugation for 5 min at 350 g. 0.02×10⁶ cells/well (50 μl/well) were plated in assay medium to obtain a final effector-to-target cell ratio (E:T) of 5:1. Subsequently, antibody dilutions were prepared in the assay medium, and were added to the 96 well plates to obtain a final volume of 200 μl. The plates were placed into the Incucyte incubator, and the acquisition of the images was set to 4 h intervals. The final readout constituted of a normalized fluorescent red area per well.

At the 72 h time point, 15 μl/well of the supernatant was collected for cytokine measurement and frozen in −20° C. On the day of the cytokine readout, supernatants were thawed 30 min prior to the assay beginning. Technical triplicates were pulled and analysed with a Bio-Plex Pro Human Cytokine 8-Plex Assay (Bio-Rad, #M50000007A), according to the manufacturer's protocol. The readout was performed with a Bio-Plex 200 system.

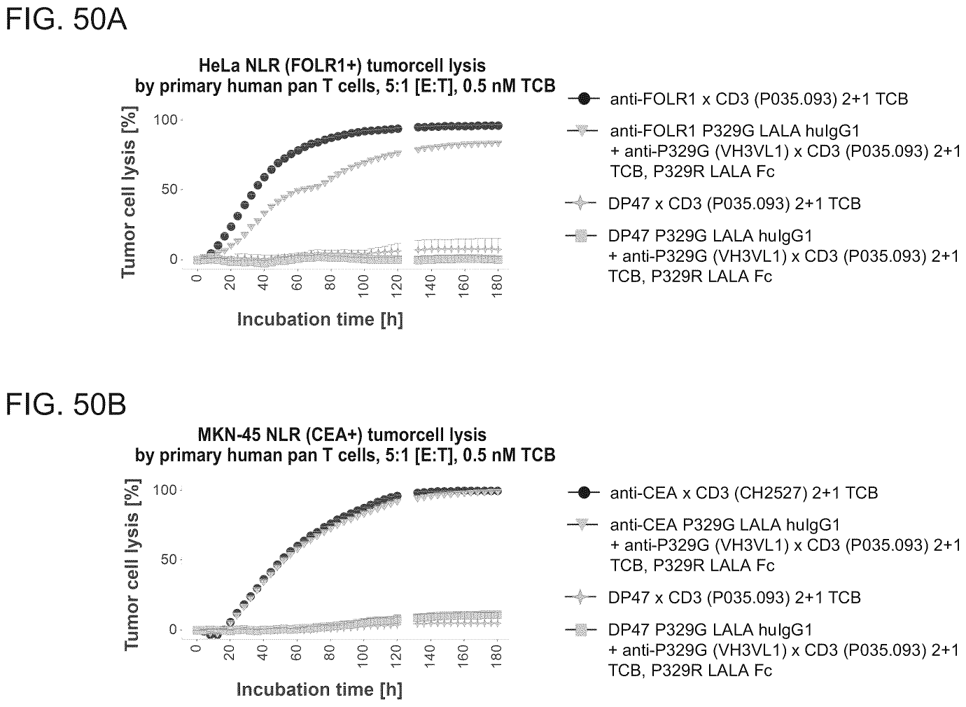

FIG. 50A-FIG. 50B depicts tumor cell lysis results from experiments performed with HeLa NLR (FIG. 50A) and MKN-45 NLR (FIG. 50B) cells. Kinetics of tumor cells by primary human pan T cells is shown. The data points show mean values of the technical triplicates of one experiment. Error bars indicate standard deviation. The only efficacious therapies are the direct tumor-targeting TCBs and anti-FolR1 (16D5) P329G LALA huIgG1 or anti-CEA (T84.66-LCHA) P329G LALA huIgG1 used in combination with the anti-P329G (VH3VL1)×CD3 (P035.093) 2+1 TCB, P329R LALA Fc.

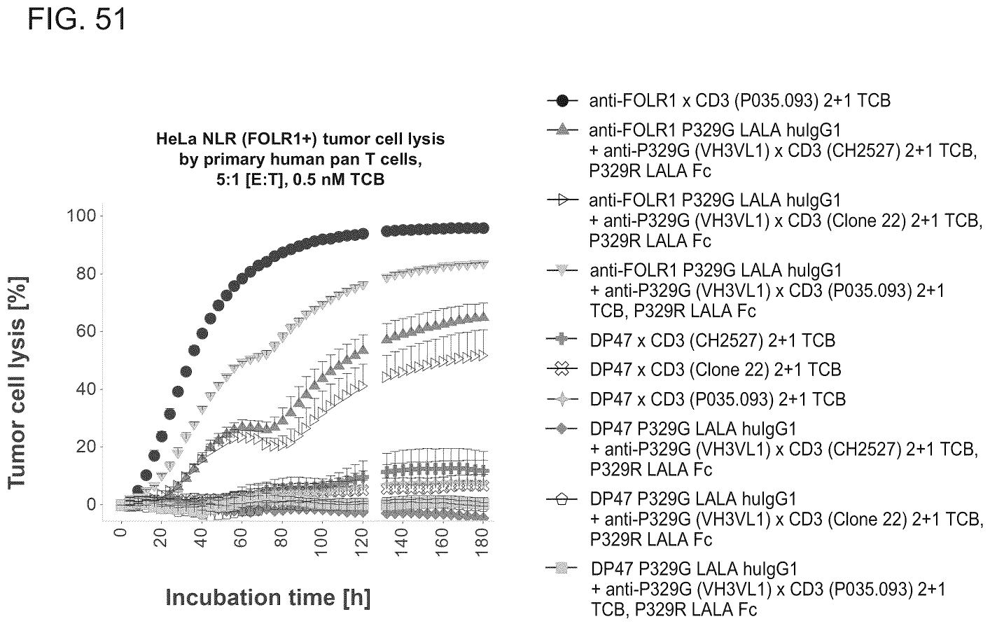

FIG. 51 depicts tumor cell lysis results from experiments performed with HeLa-NLR, comparing three different CD3 binders. The data points show mean values of the technical triplicates of one experiment. Error bars indicate standard deviation. The anti-P329G (VH3VL1)×CD3 (P035.093) 2+1 TCB, P329R LALA Fc, in combination with the anti-FOLR1 (16D5) P329G LALA huIgG1 showed the best capacity for tumor cell lysis out of all a-P329G TCBs with different CD3 binders.

### Example 12

Primary Human T Cell Activation Assay on HeLa (FOLR1+) Cells; Comparison of the CD3 Binders in the Anti-P329G TCB; Three T Cell Donors

T cell activation capacity of three a-P329G TCBs with three different CD3 binders (P035.093, CH2527, Clone 22) was assessed with primary human pan T cells or PBMCs cells from three healthy donors.

T cell activation induced by the anti-P329G (VH3VL1)×CD3 (P035.093) 2+1 TCB, P329R LALA Fc, anti-P329G (VH3VL1)×CD3 (CH2527) 2+1 TCB, P329R LALA Fc, anti-P329G (VH3VL1)×CD3 (Clone 22) 2+1 TCB, P329R LALA Fc was tested in presence of a tumor-targeting P329G LALA huIgG1.

As a tumor targeting antibody, anti-FolR1 (16D5) P329G LALA huIgG1 was used in combination with the anti-P329G (VH3VL1)×CD3 2+1 TCB, P329R LALA Fc in one of three versions (P035.093, CH2527, Clone 22) in a molar ratio IgG:TCB 2:1. A non-binding DP47 P329G LALA huIgG1 combined with either of the anti-P329G TCBs, in molar ratio IgG:TCB 2:1, was used as a negative control. As a positive control, a direct tumor-targeting anti-FolR1 (16D5)×CD3 (P035.093) 2+1 TCB was used. The target cells used were HeLa (FOLR1+). The anti-P329G TCBs were mixed with the respective IgGs at the highest concentration in the molar ratio IgG:TCB 2:1, and were titrated together. All TCBs were serially diluted 10-fold starting from 50 nM of the TCBs, resulting in a concentration range from 50 nM to 50×10⁻⁸ nM.

The primary human T cell activation assay was performed as described in Example 9.

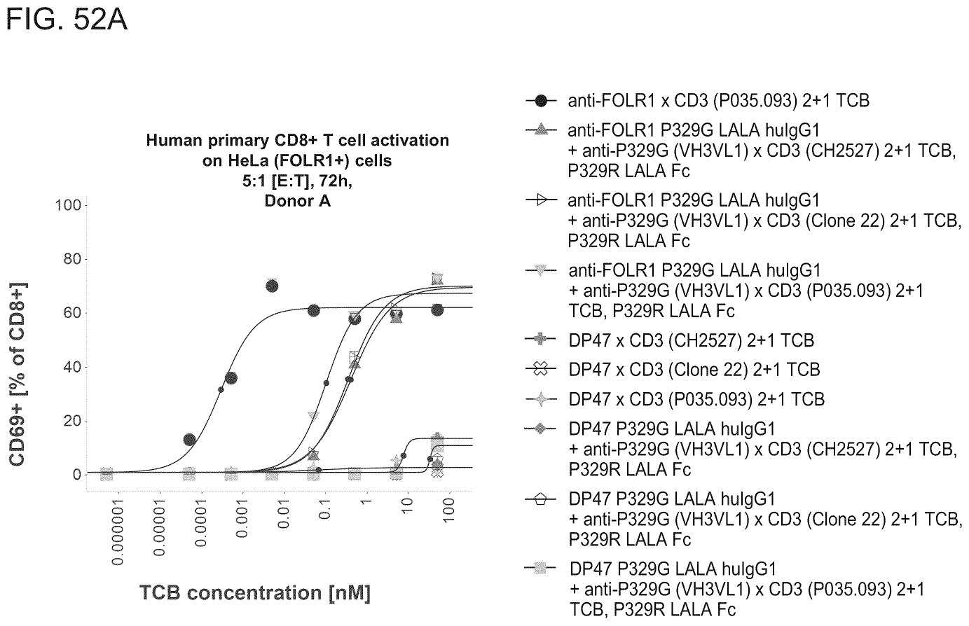

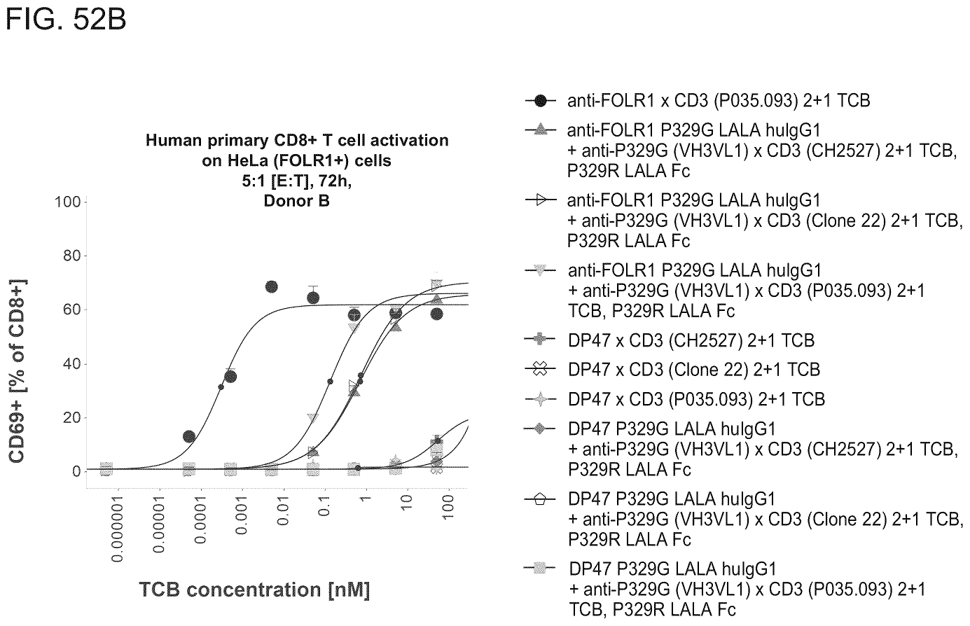

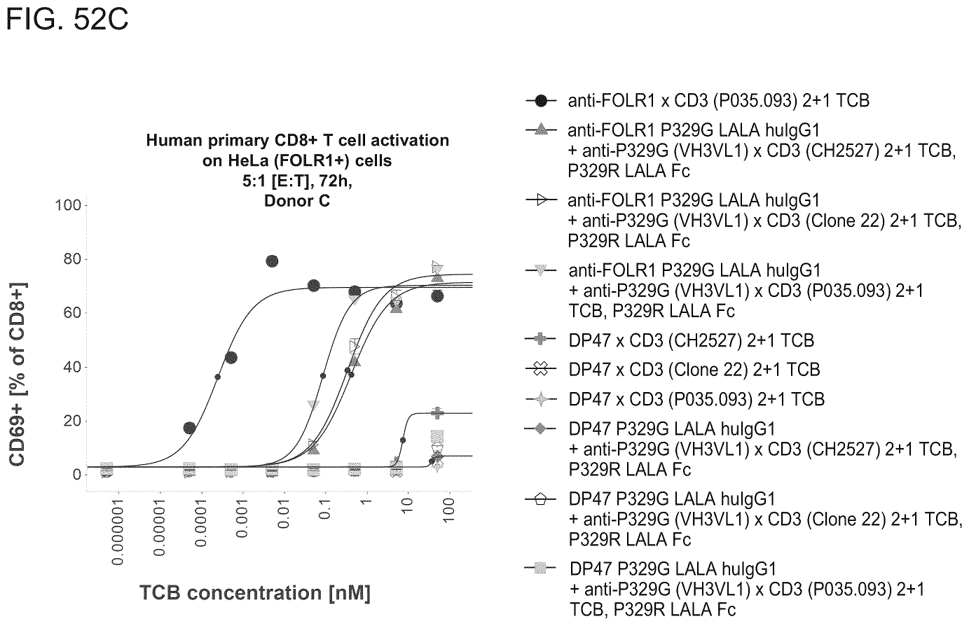

FIG. 52A-52C depicts T cell activation results from experiments performed with HeLa cells and T cells from three different healthy donors—donor A (FIG. 52A), donor B (FIG. 52B), donor C (FIG. 52C). A percentage of CD69+ T cells out of the CD8+ T cell pool was assessed as a T cell activation marker. The data points show mean values of the technical triplicates of one experiment. Error bars indicate standard deviation.

In the case of all three donors, when combined with the tumor-targeted huIgG1, the anti-P329G (VH3VL1)×CD3 (CH2527) 2+1 TCB, P329R LALA Fc shows superior activity as compared to the anti-P329G TCB versions with CH2527 and Clone 22 CD3 binders. All three CD3 binder versions of the anti-P329G TCB induce CD8+ T cell activation in a dose dependent manner.

### Example 13

Jurkat NFκB 4-1BB Activation Assay for the Assessment of Costimulatory Molecules

The capacity of the molecules anti-P329G (VH2VL1)×4-1BBL LALA huIgG1 1+1 and anti-P329G (VH2VL1)×CD28 LALA huIgG1 1+1 to induce costimulatory signaling in T cells was assessed by a Jurkat NFκB 4-1BB (Jurkat-hu4-1BB-NFκB-luc2, Promega) reporter cell line. The assay follows the same principles as the Jurkat NFAT T cell activation assay described in Example 8.

Jurkat NFκB 4-1BB reporter cells are a 4-1BBL-expressing human acute lymphatic leukemia reporter cell line with an NFκB-response element controlling the expression of firefly luciferase. Upon crosslinking of the 4-1BB, the NFκB promoter is activated, leading to a dose-dependent expression of the luciferase. Adding a luciferase substrate results in a luminescent signal, reflecting the strength of the Jurkat NFκB T cell costimulation. The crosslinking can be initiated by a bispecific antibody which simultaneously binds tumor targets and the 4-1BB on the Jurkat cells. Additionally, CD3 crosslinking (induced by e.g. a TCB) can lead to luciferase expression, due to the NFκB induction being downstream of TCR signaling in T cells.

The Jurkat NFκB 4-1BB activation induced by the anti-P329G (VH2VL1)×4-1BBL LALA huIgG1 1+1 and anti-P329G (VH2VL1)×CD28 LALA huIgG1 1+1 was tested in presence of tumor-targeting anti-CEA (T84.66-LCHA) P329G LALA huIgG1, with or without the anti-P329G (VH3VL1)×CD3 (P035.093) 2+1 TCB, P329R LALA Fc.

A non-binding DP47 P329G LALA huIgG1, combined with the anti-P329G TCB or costimulatory molecules, was used as a negative control.

The anti-P329G huIgG1s were used at a fixed concentration of 100 nM, while the anti-P329G TCB was used at a fixed concentration 0.5 nM (suboptimal dose for T cell activation). Costimulatory molecules were serially diluted 10-fold starting from 75 nM of the TCBs, resulting in a concentration range from 50 nM to 50×10⁻⁸ nM.

The assay components were incubated for 6 h in 37° C. and 5% CO₂.

The readout was performed as described in Example 8 for the single-timepoint Jurkat NFAT assay.

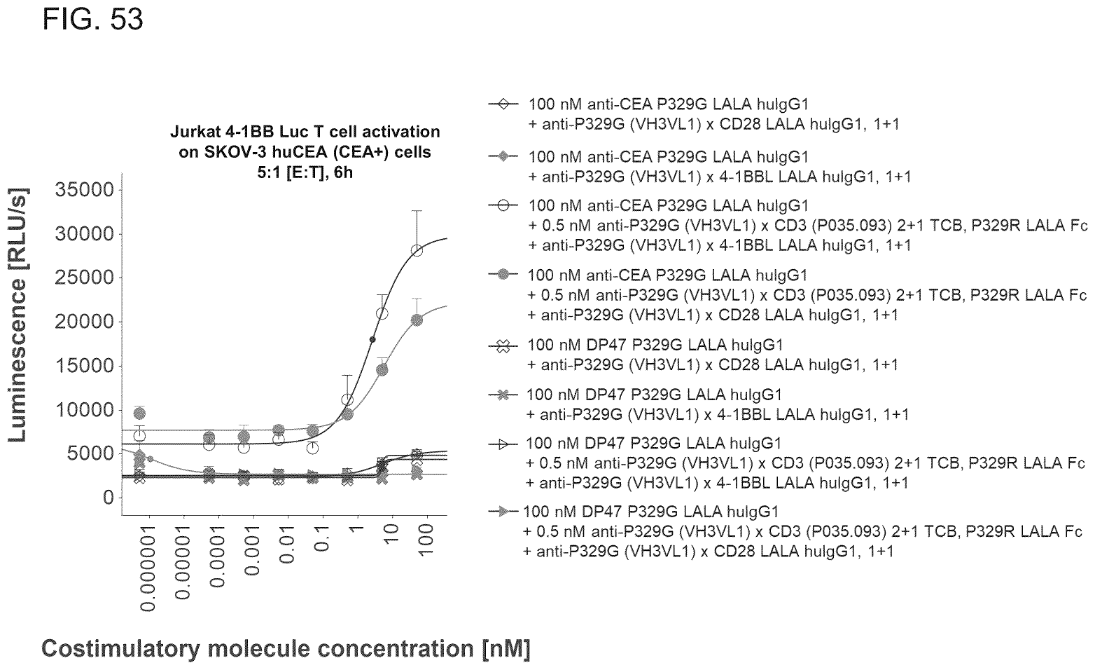

FIG. 53 depicts results from experiments performed with Jurkat NFκB 4-1BB reporter cells and SKOV-3 huCEA cells in presence of the costimulatory molecules. The data points show mean values of the technical triplicates of one experiment. Error bars indicate standard deviation. Both anti-P329G (VH2VL1)×4-1BBL LALA huIgG1 1+1 and anti-P329G (VH2VL1)×CD28 LALA huIgG1 1+1 activate the Jurkat NFκB 4-1BB cells in a dose dependent manner. Their activity is present only with a suboptimal dose of the anti-P329G (VH3VL1)×CD3 (P035.093) 2+1 TCB, P329R LALA Fc, and with simultaneous presence of the anti-CEA (T84.66-LCHA) P329G LALA huIgG1. Substituting tumor-targeted huIgG1 with a DP47 P329G LALA huIgG does not lead to activation, also when combined with the TCB, indicating a costimulation-driven effect.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An immune activating fragment crystallizable (Fc) domain binding molecule comprising
(a) an Fc domain binding moiety that specifically binds to a target Fc domain comprising a first set of at least one amino acid substitution that reduces binding affinity to an Fc receptor and/or effector function, wherein the first set of at least one amino acid substitution comprises the amino acid substitution P329G (numberings according to Kabat EU index)
(b) an immune activating moiety, and
(c) a half-life extending Fc domain, wherein the half-life extending Fc domain comprises a second set of at least one amino acid substitution that reduces binding affinity to an Fc receptor and/or effector function, wherein the second set of at least one amino acid substitution comprises a substitution at position P329 by an amino acid other than glycine (G) (numbering according to Kabat EU index),
and wherein the Fc domain binding moiety does not specifically bind to the half-life extending Fc domain.

2-8. (canceled)

9. The immune activating Fc domain binding molecule of claim 1, wherein the second set of at least one amino acid substitution comprises a substitution at position P329 (numbering according to Kabat EU index) by an amino acid selected from the list consisting of arginine (R), leucine (L), isoleucine (I), and alanine (A).

10. The immune activating Fc domain binding molecule of claim 9, wherein the second set of at least one amino acid substitution comprises a substitution at position P329 (numbering according to Kabat EU index) by arginine (R).

11. (canceled)

12. The immune activating Fc domain binding molecule of claim 1, wherein the first set of at least one amino acid substitution further comprises at least one amino acid substitution at a position selected from the group of L234 and L235 (Kabat EU index numbering).

13. The immune activating Fc domain binding molecule of claim 1, wherein the second set of at least one amino acid substitution comprises at least one amino acid substitution at a position selected from the group of L234 and L235 (Kabat EU index numbering).

14. The immune activating Fc domain binding molecule of claim 1, wherein the target Fc domain comprises three amino acid substitutions L234A, L235A and P329G (Kabat EU index numbering).

15. The immune activating Fc domain binding molecule of claim 14, wherein the half-life extending Fc domain comprises three amino acid substitutions L234A, L235A and P329X (Kabat EU index numbering), wherein X is an amino acid other than glycine (G).

16. (canceled)

17. The immune activating Fc domain binding molecule of claim 1, wherein the Fc domain binding moiety is capable of specific binding to an IgG1 Fc domain comprising the amino acid substitution P329G (numbering according to Kabat EU index), wherein the Fc domain binding moiety comprises:
A)(i) a heavy chain variable region (VH) comprising
(a) a heavy chain complementarity-determining region (CDR H) 1 comprising the amino acid sequence of RYWMN (SEQ ID NO:1);
(b) a CDR H2 comprising the amino acid sequence of EITPDSSTINYTPSLKD (SEQ ID NO:2); and
(c) a CDR H3 comprising the amino acid sequence of PYDYGAWFAS (SEQ ID NO:3); and
(ii) a light chain variable region (VL) comprising
(d) a light chain complementary-determining region (CDR L) 1 comprising the amino acid sequence of RSSTGAVTTSNYAN (SEQ ID NO:4);
(e) a CDR L2 comprising the amino acid sequence of GTNKRAP (SEQ ID NO:5); and
(f) a CDR L3 comprising the amino acid sequence of ALWYSNHWV (SEQ ID NO:6);
B)(i) a heavy chain variable region (VH) comprising
(a) a CDR H1 comprising the amino acid sequence of RYWMN (SEQ ID NO:1);
(b) a CDR H2 comprising the amino acid sequence EITPDSSTINYTPSLKG (SEQ ID NO:11); and
(c) a CDR H3 comprising the amino acid sequence of PYDYGAWFAS (SEQ ID NO:3); and
(ii) a light chain variable region (VL) comprising
(d) a CDR L1 comprising the amino acid sequence of RSSTGAVTTSNYAN (SEQ ID NO:4);
(e) a CDR L2 comprising the amino acid sequence of GTNKRAP (SEQ ID NO:5); and
(f) a CDR L3 comprising the amino acid sequence of ALWYSNHWV (SEQ ID NO:6); or
C)(i) a heavy chain variable region (VH) comprising
(a) a CDR H1 comprising the amino acid sequence of RYWMN (SEQ ID NO:1);
(b) a CDR H2 comprising the amino acid sequence EITPDSSTINYAPSLKG (SEQ ID NO:16); and
(c) a CDR H3 comprising the amino acid sequence of PYDYGAWFAS (SEQ ID NO:3); and
(ii) a light chain variable region (VL) comprising
(d) a CDR L1 comprising the amino acid sequence of RSSTGAVTTSNYAN (SEQ ID NO:4);
(e) a CDR L2 comprising the amino acid sequence of GTNKRAP (SEQ ID NO:5); and
(f) a CDR L3 comprising the amino acid sequence of ALWYSNHWV (SEQ ID NO:6).

18-20. (canceled)

21. The immune activating Fc domain binding molecule of claim 17, wherein the Fc domain binding moiety is capable of specific binding to an IgG1 Fc domain comprising the amino acid substitution P329G (numbering according to Kabat EU index), wherein the Fc domain binding moiety comprises a heavy chain variable region sequence having at least 95% identity to an amino acid sequence selected from the group consisting of SEQ ID NO:7, SEQ ID NO:12, SEQ ID NO:17 and SEQ ID NO:19, and a light chain variable region sequence having at least 95% identity to an amino acid sequence of SEQ ID NO:8 or SEQ ID NO:13.

22. The immune activating Fc domain binding molecule of claim 21, wherein the Fc domain binding moiety is capable of specific binding to an IgG1 Fc domain comprising the amino acid substitution P329G (numbering according to Kabat EU index), wherein the Fc domain binding moiety comprises
(i) a heavy chain variable region sequence having at least 95% identity to SEQ ID NO: 7 and a light chain variable region sequence having at least 95% identity to SEQ ID NO: 8,
(ii) a heavy chain variable region sequence having at least 95% identity to SEQ ID NO: 12 and a light chain variable region sequence having at least 95% identity to SEQ ID NO: 13,
(iii) a heavy chain variable region sequence having at least 95% identity to SEQ ID NO: 17 and a light chain variable region sequence having at least 95% identity to SEQ ID NO: 13, or
(iv) a heavy chain variable region sequence having at least 95% identical to SEQ ID NO: 19 and a light chain variable region sequence having at least 95% identity to SEQ ID NO: 13.

23. (canceled)

24. The immune activating Fc domain binding molecule of claim 1, wherein the Fc domain binding moiety comprises a first Fab molecule and the immune activating moiety comprises a second Fab molecule.

25. The immune activating Fc domain binding molecule according to claim 24, further comprising d) a third Fab molecule which specifically binds to the target Fc domain comprising the first set of at least one amino acid substitution.

26. (canceled)

27. The immune activating Fc domain binding molecule of claim 1, wherein the immune activating moiety is capable of specific binding to an activating T cell antigen.

28. The immune activating Fc domain binding molecule of claim 27, wherein the activating T cell antigen is CD3.

29-31. (canceled)

32. The immune activating Fc domain binding molecule of claim 28, wherein the immune activating moiety comprises
(i) a CDR H1 comprising the amino acid sequence of SEQ ID NO: 35, a CDR H2 comprising the amino acid sequence of SEQ ID NO: 37, a CDR H3 comprising the amino acid sequence of SEQ ID NO: 43, a CDR L1 comprising the amino acid sequence of SEQ ID NO: 53, a CDR L2 comprising the amino acid sequence of SEQ ID NO: 54 and a CDR L3 comprising the amino acid sequence of SEQ ID NO: 55;
(ii) a CDR H1 comprising the amino acid sequence of SEQ ID NO: 35, a CDR H2 comprising the amino acid sequence of SEQ ID NO: 37, a CDR H3 comprising the amino acid sequence of SEQ ID NO: 176, a CDR L1 comprising the amino acid sequence of SEQ ID NO: 53, a CDR L2 comprising the amino acid sequence of SEQ ID NO: 54 and a CDR L3 comprising the amino acid sequence of SEQ ID NO: 55; or
(iii) a CDR H1 comprising the amino acid sequence of SEQ ID NO: 34, a CDR H2 comprising the amino acid sequence of SEQ ID NO: 37, a CDR H3 comprising the amino acid sequence of SEQ ID NO: 41, a CDR L1 comprising the amino acid sequence of SEQ ID NO: 53, a CDR L2 comprising the amino acid sequence of SEQ ID NO: 54 and a CDR L3 comprising the amino acid sequence of SEQ ID NO: 55.

33. The immune activating Fc domain binding molecule of claim 32, wherein the immune activating moiety comprises
(i) a heavy chain variable region comprising an amino acid sequence having at least 95% identity to the amino acid sequence of SEQ ID NO: 49 and a light chain variable region comprising an amino acid sequence having at least 95% identity to the amino acid sequence of SEQ ID NO: 56;
(ii) a heavy chain variable region comprising an amino acid sequence having at least 95% identity to the amino acid sequence of SEQ ID NO: 177 and a light chain variable region comprising an amino acid sequence having at least 95% identity to the amino acid sequence of SEQ ID NO: 56; or
(iii) a heavy chain variable region comprising an amino acid sequence having at least 95% identity to the amino acid sequence of SEQ ID NO: 47 and a light chain variable region comprising an amino acid sequence having at least 95% identity to the amino acid sequence of SEQ ID NO: 56.

34-37. (canceled)

38. An immune activating domain binding molecule of claim 33, comprising:
(i) (a) a first light chain comprising an amino acid sequence having at least 95% identity to SEQ ID NO:89;
(b) a second light chain comprising the amino acid sequence having at least 95% identity to SEQ ID NO:70;
(c) a first heavy chain comprising the amino acid sequence having at least 95% identity to SEQ ID NO:178; and
(d) a second heavy chain comprising the amino acid sequence having at least 95% identity to SEQ ID NO:179;
(ii) (a) a first light chain comprising an amino acid sequence having at least 95% identity to SEQ ID NO:89;
(b) a second light chain comprising the amino acid sequence having at least 95% identity to SEQ ID NO:68;
(c) a first heavy chain comprising the amino acid sequence having at least 95% identity to SEQ ID NO:178; and
(d) a second heavy chain comprising the amino acid sequence having at least 95% identity to SEQ ID NO:179; or
(iii) (a) a first light chain comprising an amino acid sequence having at least 95% identity to SEQ ID NO:89;
(b) a second light chain comprising the amino acid sequence having at least 95% identity to SEQ ID NO:180;
(c) a first heavy chain comprising the amino acid sequence having at least 95% identity to SEQ ID NO:178; and
(d) a second heavy chain comprising the amino acid sequence having at least 95% identity to SEQ ID NO:179.

39-40. (canceled)

41. The immune activating Fc domain binding molecule of claim 1, wherein the immune activating moiety is capable of specific binding to a costimulatory T cell antigen, wherein the costimulatory T cell antigen is CD28.

42-45. (canceled)

46. The immune activating Fc domain binding molecule of claim 41, wherein the immune activating moiety comprises
(i) a CDR H1 comprising the amino acid sequence of SEQ ID NO: 94, a CDR H2 comprising the amino acid sequence of SEQ ID NO: 95, a CDR H3 comprising the amino acid sequence of SEQ ID NO: 96, a CDR L1 comprising the amino acid sequence of SEQ ID NO: 97, a CDR L2 comprising the amino acid sequence of SEQ ID NO: 98 and a CDR L3 comprising the amino acid sequence of SEQ ID NO: 99; or
(ii) a CDR H1 comprising the amino acid sequence of SEQ ID NO: 94, a CDR H2 comprising the amino acid sequence of SEQ ID NO: 95, a CDR H3 comprising the amino acid sequence of SEQ ID NO: 102, a CDR L1 comprising the amino acid sequence of SEQ ID NO: 103, a CDR L2 comprising the amino acid sequence of SEQ ID NO: 98 and a CDR L3 comprising the amino acid sequence of SEQ ID NO: 99

47. The immune activating Fc domain binding molecule of claim 46, wherein the immune activating moiety comprises a heavy chain variable region comprising an amino acid sequence having at least 95% identity to the amino acid sequence of SEQ ID NO: 100 and a light chain variable region comprising an amino acid sequence having at least 95% identity to the amino acid sequence of SEQ ID NO: 101; or a heavy chain variable region comprising an amino acid sequence having at least 95% identity to the amino acid sequence of SEQ ID NO: 104 and a light chain variable region comprising an amino acid sequence having at least 95% identity to the amino acid sequence of SEQ ID NO: 105.

48. The immune activating Fc domain binding molecule of claim 1, wherein the immune activating moiety is capable of specific binding to a costimulatory T cell antigen, where the costimulatory T cell antigen is 4-1BB.

49-52. (canceled)

53. The immune activating Fc domain binding molecule of claim 48, wherein the immune activating moiety comprises a CDR H1 comprising the amino acid sequence of SEQ ID NO: 133, a CDR H2 comprising the amino acid sequence of SEQ ID NO: 134, a CDR H3 comprising the amino acid sequence of SEQ ID NO: 135, a CDR L1 comprising the amino acid sequence of SEQ ID NO: 136, a CDR L2 comprising the amino acid sequence of SEQ ID NO: 137 and a CDR L3 comprising the amino acid sequence of SEQ ID NO: 138.

54. The immune activating Fc domain binding molecule of claim 53, wherein the immune activating moiety comprises a heavy chain variable region comprising an amino acid sequence having at least 95% identity to the amino acid sequence of SEQ ID NO: 139 and a light chain variable region comprising an amino acid sequence having at least 95% identity to the amino acid sequence of SEQ ID NO: 140.

55. The immune activating Fc domain binding molecule of claim 1, wherein the immune activating moiety is a cytokine selected from the group consisting of IL2, IL7, IL15, IL18, IFNa and IFNg.

56. (canceled)

57. The immune activating Fc domain binding molecule of claim 55, wherein the immune activating moiety is a mutant interleukin-2 (IL-2) polypeptide, wherein the mutant IL-2 polypeptide is a human IL-2 molecule comprising the amino acid substitutions F42A, Y45A and L72G (numbering relative to the human IL-2 sequence SEQ ID NO:166).

58-61. (canceled)

62. The immune activating Fc domain binding claim 1, wherein the immune activating moiety comprises three ectodomains of 4-1BBL or a fragment thereof.

63. The immune activating Fc domain binding molecule of claim 62, wherein the immune activating moiety comprises a first and a second polypeptide, wherein the first polypeptide contains a first heavy chain constant (CH1) or a light chain constant (CL) domain and the second polypeptide contains a CL or CH1 domain, respectively, wherein the second polypeptide is linked to the first polypeptide by a disulfide bond between the CH1 and CL domain, and wherein the first polypeptide comprises two ectodomains of 4-1BBL or a fragment thereof that are connected to each other and to the CH1 or CL domain by a peptide linker and wherein the second polypeptide comprises one ectodomain of said 4-1BBL or a fragment thereof connected via a peptide linker to the CL or CH1 domain of said polypeptide.

64-67. (canceled)

68. The immune activating Fc domain binding molecule of claim 1, wherein the immune activating moiety is capable of specific binding to an Fc receptor.

69. (canceled)

70. The immune activating Fc domain binding molecule of claim 68, wherein the Fc receptor is CD16.

71. One or more isolated polynucleotides encoding the immune activating Fc domain binding molecule of claim 1.

72. One or more vectors comprising the one or more polynucleotides of claim 71.

73. A host cell comprising the one or more polynucleotides of claim 71 or the one or more vectors of claim 72.

74. A method of producing an immune activating Fc domain binding molecule, comprising the steps of a) culturing the host cell of claim 73 under conditions suitable for the expression of the immune activating Fc domain binding molecule and b) recovering the immune activating Fc domain binding molecule.

75. (canceled)

76. A pharmaceutical composition comprising the immune activating Fc domain binding molecule of claim 1 and a pharmaceutically acceptable carrier.

77-83. (canceled)

84. A method of treating a disease in an individual, comprising administering to said individual a therapeutically effective amount of a composition comprising the immune activating Fc domain binding molecule of claim 1 in a pharmaceutically acceptable form.

85. The method of claim 84, wherein said disease is cancer.

86. The method of claim 84 further comprising
administering to said individual a therapeutically effective amount of a composition comprising a targeting antibody comprising the target Fc domain.

87-89. (canceled)

90. A method of inducing lysis of a cell, comprising contacting the cell with the immune activating Fc domain binding molecule of claim 1 and a targeting antibody comprising the target Fc domain in the presence of a T cell, wherein the targeting antibody is capable of specific binding to an antigen on the cell.

91. (canceled)
